(19) United States
(12) Patent Application Publication
(10) Pub. No.: US 2026/0152487 A1
(43) Pub. Date: Jun. 4, 2026

(54) PHTHALAZINE DERIVATIVES AS PYRUVATE KINASE MODULATORS

(71) Applicant: Sitryx Therapeutics Limited, Oxford (GB)

(72) Inventors: David COUSIN, Nottingham, Nottinghamshire (GB); Oscar BARBA, Oxford (GB)

(73) Assignee: Sitryx Therapeutics Limited, Oxford (GB)

(21) Appl. No.: 18/707,418

(22) PCT Filed: Nov. 4, 2022
(86) PCT No.: PCT/GB2022/052781

§ 371 Date: May 3, 2024

(30) Foreign Application Priority Data
Nov. 5, 2021 (EP) 21206793.8
Dec. 22, 2021 (EP) 21216843.9
Jun. 8, 2022 (EP) 22177827.7

(51) Int. Cl. C07D 403/06; A61K 31/502; A61K 31/5025; A61K 31/538; A61K 31/5383; A61K 45/06; A61P 35/00; C07D 237/32; C07D 401/06; C07D 401/12; C07D 401/14; C07D 403/12; C07D 403/14; C07D 405/06; C07D 405/12; C07D 405/14; C07D 409/06; C07D 409/12; C07D 409/14; C07D 413/06; C07D 413/14; C07D 417/06; C07D 417/12; C07D 417/14; C07D 471/04; C07D 491/048; C07D 491/056; C07D 498/04; C07D 519/00

(52) U.S. Cl. CPC C07D 403/06; A61K 31/502; A61K 31/5025; A61K 31/538; A61K 31/5383; A61K 45/06; A61P 35/00; C07D 237/32; C07D 401/06; C07D 401/12; C07D 401/14; C07D 403/12; C07D 403/14; C07D 405/06; C07D 405/12; C07D 405/14; C07D 409/06; C07D 409/12; C07D 409/14; C07D 413/06; C07D 413/14; C07D 417/06; C07D 417/12; C07D 417/14; C07D 471/04; C07D 491/048; C07D 491/056; C07D 498/04; C07D 519/00

(57) ABSTRACT

The invention relates to compounds of formula (Ia) and to their use in treating or preventing an inflammatory disease, a disease associated with an undesirable immune response, cancer, obesity, a diabetic disease or a blood disorder:

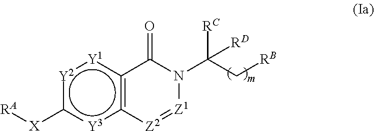

wherein R^{A}, R^{B}, R^{C} and R^{D}, X, Y¹, Y², Y³, Z¹, Z² and m are as defined herein.

### FIELD OF THE INVENTION

The present invention relates to compounds and their use in treating or preventing an inflammatory disease, a disease associated with an undesirable immune response, cancer, obesity, a diabetic disease or a blood disorder, and to related compositions, methods and intermediate compounds.

### BACKGROUND OF THE INVENTION

Pyruvate kinase (PK) is the enzyme responsible for the final rate-limiting step of glycolysis, catalyzing phosphoenolpyruvic acid (PEP) and ADP to pyruvate and ATP. Four PK isoforms exist in mammals from two separate genes (Alves-Filho et al., 2016). PKL and PKR, products of the Pklr gene, are expressed in the liver and red blood cells, respectively. PKM1 and 2 are alternatively spliced products of the Pkm gene. PKM1 is expressed in tissues with high energy demands such as heart, muscle, and brain, and PKM2 is expressed in embryonic tissues, cancer and normal proliferating cells such as lymphocytes and intestinal epithelial cells. Whereas PKM1 is a constitutively active enzyme, PKM2 is a low-activity enzyme that relies on allosteric activation by multiple endogenous regulators, for example, the upstream glycolytic intermediate, fructose-1,6-bisphosphate (FBP). Binding of these allosteric regulators induces conformational changes that promote tetramerization of PKM2 leading to an increase in the last rate-limiting step of glycolysis. Pyruvate will enter the TCA cycle in the mitochondria where it is used to generate ATP through oxidative phosphorylation. Without allosteric activation PKM2 takes on a dimeric or monomeric form with low enzymatic activity, leading to accumulation of glycolytic intermediates which meet the requirements for biosynthetic precursors of the activated or proliferating cell. Dimeric PKM2 can also translocate to the nucleus where it can further promote aerobic glycolysis and regulate transcriptional activity, acting as a protein kinase to target transcription factors and histones.

Cancer cells primarily use glycolysis to generate cellular energy and biosynthesis intermediates, termed the Warburg effect and PKM2 plays a dominant role in glycolysis to achieve the nutrient demands of cancer cell proliferation (Chhipa et al., 2018). PKM2 is overexpressed in almost all cancers and has been shown to promote proliferation and metastasis of tumour cells. In addition to controlling glycolytic flux, the non-metabolic role of PKM2 as a coactivator and protein kinase contribute to tumorigenesis (Dong et al., 2016). PKM2 binds directly to and phosphorylates histone H3 leading to expression of c-Myc and Cyclin D1 and the proliferation of cancer cells. Activation of PKM2 tetramer by small molecules could be an attractive therapy in cancer to contain tumour growth by preventing the non-metabolic functions of dimeric PKM2.

Following activation or an inflammatory stimulus, PKM2 is upregulated in many immune cells including macrophages and T cells (Pålsson-McDermott et al., 2020). The non-metabolic roles of dimeric PKM2 have been shown to regulate immune responses: PKM2 acts as a transcriptional coactivator of Hif-1α, b-catenin and STAT3 leading to expression of pro-inflammatory cytokines such as IL-1β and TNFα. Activation of PKM2 by small molecules to prevent nuclear translocation could have therapeutic benefit in a range of inflammatory and auto-immune conditions, such as rheumatoid arthritis, inflammatory bowel diseases, inflammatory skin pathologies, coronary artery disease and multiple sclerosis.

In diabetes, PKM2 regulates glucose responsive pancreatic beta-cell function and protects from metabolic stress (Abulizi et al., 2020; Lewandowski et al., 2020). Dimeric PKM2 plays a role in aberrant glycolysis by promoting the accumulation of HIF-1a, and in diabetic nephropathy PKM2 is associated with a pathogenic role in glomerular injury and epithelial-to-mesenchymal transition leading to fibrosis (Liu et al., 2020). PKM2 activation has been shown to amplify insulin release and improve insulin sensitivity and protect against progression of diabetic glomerular pathology and kidney fibrosis (Liu et al., 2020; Abulizi et al., 2020; Lewandowski et al., 2020; Qi et al., 2017).

Obesity is defined as abnormal or excessive fat accumulation that presents a risk to health, and is linked to a higher incidence of type 2 diabetes and cardiovascular disease. This metabolic disorder is strongly associated with insulin resistance and the adverse impact on glucose metabolism and disposal in obese subjects (Barazzoni et al., 2018). Studies on 3T3-L1 adipocytes exposed to varying levels of insulin resulted in significant increases in PKM2 mRNA levels, independent of the levels of glucose in the media (Puckett et al., 2021). Work on the impact of altered PKM2 phosphorylation status and resulting decreased catalytic activity, has identified PKM2 as a potential contributor to insulin resistance in the adipose tissue and made an association with metabolic status in humans (Bettaieb et al., 2013). Restoring PKM2 activity with a small molecule allosteric activator has been shown to improve insulin sensitivity (Abulizi et al. 2020; Lewandowski et al. 2020) and warrants further investigation as a novel target for pharmacological intervention in obesity.

Pyruvate kinase deficiency (PKD) is one of the most common enzyme defects in erythrocytes, that presents as hemolytic anemia, the accelerated destruction of red blood cells (Bianchi et al., 2020). Mature red blood cells depend entirely on glycolysis for maintaining cell integrity and function, and so pyruvate kinase plays a crucial role in erythrocyte metabolism and survival. The inherited mutations in PKR enzymes lead to dysregulation of its catalytic activity and cause a deficit in cellular energy within the red blood cell, as evidenced by lower pyruvate kinase enzyme activity, a decline in ATP levels and a build-up of upstream metabolites. PKR decreased activity has also been linked to changes in the erythrocytes morphology and cell membrane surface suggesting a wider involvement of this enzyme in the entire lifespan of these cells (Cançado et al., 2018). PK-deficient erythrocytes are prematurely removed from the circulation by the spleen through accelerated hemolysis leading to iron accumulation. Increase and/or restoration of PKR activity to quasi-basal levels is thought to have potential to treat the PK deficiency-related complications. The current standard of care for PKD is supportive, including blood transfusions, splenectomy, chelation therapy to address iron overload and/or interventions for other treatment- and disease-related morbidities. There is no approved therapy to treat the underlying cause of PK deficiency. Activation of the PKR enzyme with a small molecule allosteric activator increases PK enzyme activity and enhanced glycolysis in erythrocytes from patients with PK deficiency (Kung et al., 2017).

Pharmacological intervention by using small molecules agonists such as TEPP-46 and DASA-58 have been utilised extensively in vitro and in vivo biological settings to demonstrate the several potential benefits provided by augmenting PK activity through allosteric modulation (Yi et al., 2021). Although these compounds show a good level of in vitro activity, their ADME and pharmacokinetic/pharmacodynamic profiles have prevented them from being developed for the treatment of human disease. The structure of TEPP-46 is as follows:

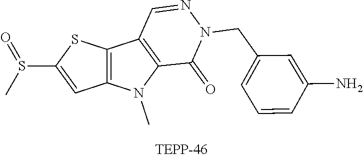 [SMILES: Cn1c2cc(S(C)=O)sc2c2cnn(Cc3cccc(N)c3)c(=O)c21]

The most advanced PK activator being extensively studied in clinical settings is Mitapivat (AG-348), a PKM2 and PKLR activator being investigated for the treatment of several blood disorders arising from PK mutant forms that exhibit lower catalytic activity than corresponding wild type red blood cells (Kung et al., 2017). This agent has shown an adequate level of efficacy when dosed in patients presenting PK deficiency by increasing basal haemoglobin levels. However, despite the promising results, the high dosing regimen and the BID (two times a day) dosing frequency needed to achieve efficacy have highlighted the need to develop more efficacious compounds with a more favourable pharmacokinetic and improved safety profile (Grace et al., 2019). The structure of mitapivat is as follows:

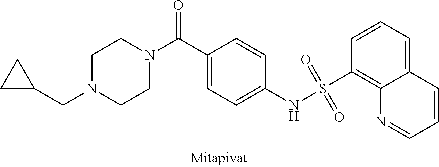 [SMILES: O=C(c1ccc(NS(=O)(=O)c2cccc3cccnc23)cc1)N1CCN(CC2CC2)CC1]

WO2020/167976A1 (Agios Pharmaceuticals, Inc.) describes compounds that are said to regulate PK activity, for the treatment of cancer, obesity and diabetes related disorders.

There remains a need to identify and develop new disease modifying PK modulators to meet several unmet medical needs linked to PK disfunction, in particular the need to develop compounds that demonstrate suitable activity while also having favourable physical-chemical parameters. The compounds herein described as PK modulators, in particular PKM2 and/or PKLR modulators, in particular PKM2 and/or PKLR activators, address the aforementioned unmet needs by exhibiting suitable affinity and functional activity for PK enzymes, in particular PKM2 and/or PKLR, while having better overall physical/chemical properties with improved ADME and PK profiles making them suitable for the treatment of human diseases linked to an altered function of pyruvate kinase enzymes expression and/or activity.

### SUMMARY OF THE INVENTION

The present invention provides a compound of formula (Ia):

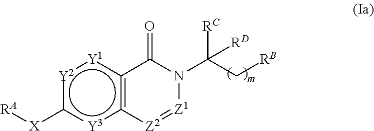

wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available atom by one or more R^{1A}, wherein each R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A}, NHCOCH₃, CO₂H, CH₂CO₂H, CONR^{2A}R^{3A}, CH₂CONR^{2A}R^{3A}, C₃₋₆ cycloalkyl, oxo, and a 4 membered heterocyclic ring, wherein the 4 membered heterocyclic ring is optionally substituted by hydroxy;
   R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
   wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
      wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A},
         wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
            or
Y² is absent and one of Y¹ and Y³ is S, O or NR^{5A}, and the remaining Y¹ or Y³ is N or CR^{6A}; wherein R^{5A} is H or C₁₋₃ alkyl; and
   R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
      wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A},
         wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available atom by one or more R^{1B}, wherein each R^{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl, oxo, and a 4 membered heterocyclic ring, wherein the 4 membered heterocyclic ring is optionally substituted by hydroxy;
   R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —C(R^{E})(R^{F})O or CH₂NH—; and
R^{E} and R^{F} are H, or R^{E} and R^{F} join to form a C₃₋₅ heterocycloalkyl ring; or a pharmaceutically acceptable salt and/or solvate thereof.

Compounds of formula (Ia) are useful in therapy, in particular in the treatment of PK-mediated diseases, disorders and conditions. Compounds of formula (Ia) may be activators of the PK family of proteins, especially activators of PKM2 and/or PKLR.

The present invention provides a pharmaceutical composition comprising a compound of formula (Ia) or a pharmaceutically acceptable salt and/or solvate thereof.

The present invention provides a compound of formula (Ia) or a pharmaceutically acceptable salt and/or solvate thereof for use as a medicament.

The present invention provides a compound of formula (Ia) or a pharmaceutically acceptable salt and/or solvate thereof for use in treating or preventing a disease, disorder or condition associated with the function of PK, in particular PKM2 and/or PKLR.

The present invention provides a compound of formula (Ia) or a pharmaceutically acceptable salt and/or solvate thereof for use in treating or preventing an inflammatory disease, a disease associated with an undesirable immune response, cancer, obesity, a diabetic disease or a blood disorder.

The present invention provides the use of a compound of formula (Ia) or a pharmaceutically acceptable salt and/or solvate thereof in the manufacture of a medicament for treating or preventing an inflammatory disease, a disease associated with an undesirable immune response, cancer, obesity, a diabetic disease or a blood disorder.

The present invention provides a method of treating or preventing an inflammatory disease, a disease associated with an undesirable immune response, cancer, obesity, a diabetic disease or a blood disorder, which comprises administering a compound of formula (Ia) or a pharmaceutically acceptable salt and/or solvate thereof.

Also provided are intermediate compounds of use in the preparation of compounds of formula (Ia).

### DETAILED DESCRIPTION OF THE INVENTION

### Compounds of Formula (Ia)

In one embodiment, the compound of formula (Ia) is a compound of formula (I):

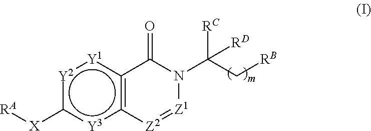

wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available carbon atom by one or more R^{1A}, wherein R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A}, NHCOCH₃, CO₂H, CH₂CO₂H, CONR^{2A}R^{3A}, CH₂CONR^{2A}R^{3A}, C₃₋₆ cycloalkyl and oxo;
   R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
   wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
      wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A},
         wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
            or
Y² is absent and one of Y¹ and Y³ is S, O or NR^{5A}, and the remaining Y¹ or Y³ is N or CR^{6A}; wherein R^{5A} is H or C₁₋₃ alkyl; and
   R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
      wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A},
         wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available carbon atom by one or more R^{1B}, wherein R_{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl and oxo;
   R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —CH₂O— or CH₂NH—;
   or a pharmaceutically acceptable salt and/or solvate thereof.

In one embodiment, the compound of formula (Ia) is a compound of formula (Ib):

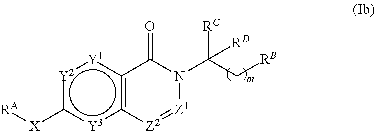

wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available carbon or nitrogen atom by one or more R^{1A}, wherein each R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A}, NHCOCH₃, CO₂H, CH₂CO₂H, CONR^{2A}R^{3A}, CH₂CONR^{2A}R^{3A}, C₃₋₆ cycloalkyl and oxo;
   R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
   wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
      wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and N^{R10A}R^{11A},
         wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
            or
Y² is absent and one of Y¹ and Y³ is S, O or NR^{5A}, and the remaining Y¹ or Y³ is N or CR^{6A};
   wherein R^{5A} is H or C₁₋₃ alkyl; and
   R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
      wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A},
         wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available carbon or nitrogen atom by one or more R^{1B}, wherein each R^{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl and oxo;
   R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —CH₂O— or CH₂NH—;
   or a pharmaceutically acceptable salt and/or solvate thereof.

Embodiments and preferences set out herein with respect to the compound of formula (Ia) apply equally to the pharmaceutical composition, compound for use, use, method and process aspects of the invention.

The term “C₁₋₆ alkyl” refers to a straight or branched fully saturated hydrocarbon group having from 1 to 6 carbon atoms. The term encompasses methyl, ethyl, n-propyl, isopropyl, n-butyl, isobutyl, sec-butyl, tert-butyl, n-pentyl and n-hexyl. C₁₋₅ alkyl, C₁₋₄ alkyl, C₁₋₃ alkyl, C₁₋₂ alkyl, C₂₋₆ alkyl, C₂₋₅ alkyl, C₂₋₄ alkyl, C₂₋₃ alkyl, C₃₋₆ alkyl, C₃₋₅ alkyl, C₃₋₄ alkyl, C₄₋₆ alkyl, C₄₋₅ alkyl and C₅₋₆ alkyl are as defined above but contain different numbers of carbon atoms. The term “C₁₋₆ alkyl” also encompasses “C₁₋₆ alkylene” which is a bifunctional straight or branched fully saturated hydrocarbon group having from 1 to 6 carbon atoms. Example “C₁₋₆ alkylene” groups include methylene, ethylene, n-propylene, n-butylene, n-pentylene and n-hexylene.

The term “C₂₋₆ alkenyl” refers to a straight or branched hydrocarbon group having from 2 to 6 carbon atoms and at least one carbon-carbon double bond. The term encompasses, CH═CH₂, CH₂CH═CH₂, CH═CHCH₃, CH₂CH₂CH═CH₂, CH═CHCH₂CH₃, CH₂CH═CHCH₃, CH₂CH₂CH₂CH═CH₂, CH═CHCH₂CH₂CH₃, CH₂CH═CHCH₂CH₃, CH₂CH₂CH═CHCH₃, CH═CHCH═CHCH₃ and CH₂CH═CHCH═CH₂. Branched variants such as CH(CH₃)CH═CH₂ and CH═C(CH₃)₂ are also included. Other alkenyl groups, for example C₂₋₅ alkenyl, C₂₋₄ alkenyl, C₂₋₃ alkenyl, C₃₋₆ alkenyl, C₃₋₅ alkenyl, C₃₋₄ alkenyl, C₄₋₆ alkenyl, C₄₋₅ alkenyl and C₅₋₆ alkenyl are as defined above but contain different numbers of carbon atoms. The term “C₂₋₆ alkenyl” also encompasses “C₂₋₆ alkenylene” which is a bifunctional straight or branched hydrocarbon group having from 2 to 6 carbon atoms and at least one carbon-carbon double bond. Example “C₂₋₆ alkenylene” groups include ethenylene, n-propenylene, n-butenylene, n-heptenylene and n-hexenylene.

The term “C₂₋₆ alkynyl” refers to a straight or branched hydrocarbon group having from 2 to 6 carbon atoms and at least one carbon-carbon triple bond. The term encompasses, C≡CH, CH₂C≡CH, C≡CCH₃, CH₂CH₂C≡CH, C≡CCH₂CH₃, CH₂C≡CCH₃, CH₂CH₂CH₂C≡CH, C≡CCH₂CH₂CH₃, CH₂C≡CCH₂CH₃, CH₂CH₂C≡CCH₃, C≡CC≡CCH₃ and CH₂C≡CC≡CH. Branched variants such as CH(CH₃)C≡CH are also included. Other alkynyl groups, for example C₂₋₅ alkynyl, C₂₋₄alkynyl, C₂₋₃ alkynyl, C₃₋₆ alkynyl, C₃₋₅ alkynyl, C₃₋₄alkynyl, C₄₋₆ alkynyl, C₄₋₅ alkynyl and C₅₋₆ alkynyl are as defined above but contain different numbers of carbon atoms. The term “C₂₋₆ alkynyl” also encompasses “C₂₋₆ alkynylene” which is a bifunctional straight or branched hydrocarbon group having from 2 to 6 carbon atoms and at least one carbon-carbon triple bond. Example “C₂₋₆ alkynylene” groups include ethynylene, n-propynylene, n-butynylene, n-heptynylene and n-hexynylene.

The term “C₁₋₆ haloalkyl” (e.g. C₁₋₅ haloalkyl, C₁₋₄ haloalkyl, C₁₋₃ haloalkyl, C₁₋₂ haloalkyl or C₁ haloalkyl) refers to a straight or a branched fully saturated hydrocarbon group containing the specified number of carbon atoms and at least one halogen atom, such as fluoro or chloro, especially fluoro. An example of haloalkyl is CF₃. Further examples of haloalkyl are CHF₂ and CH₂CF₃.

The term “C₃₋₆ cycloalkyl” (such as C₃₋₄ cycloalkyl, C₃₋₅ cycloalkyl, C₄₋₅ cycloalkyl, C₄₋₆ cycloalkyl and C₅₋₆ cycloalkyl) refers to a fully saturated cyclic hydrocarbon group having from 3 to 6 carbon atoms. The term encompasses cyclopropyl, cyclobutyl, cyclopentyl and cyclohexyl.

The term “5-7 membered heterocyclic ring” refers to a non-aromatic cyclic group having 5 to 7 ring atoms, at least one of which is a heteroatom selected from N, O, S and B. The term “heterocyclic ring” is interchangeable with “heterocyclyl”. The term encompasses pyrrolidinyl, tetrahydrofuranyl, tetrahydrothienyl, tetrahydropyranyl, piperidinyl, piperazinyl, morpholinyl and thiomorpholinyl. Other heterocyclyl groups, for example 6-7 membered heterocyclyl, 5-6 membered heterocyclyl, 5 membered heterocyclyl, 6 membered heterocyclyl and 7 membered heterocyclyl are as defined above but contain different numbers of ring atoms. Bicyclic heterocyclic compounds are also encompassed, such as the following:

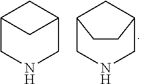

The term “4 membered heterocyclic ring” refers to a non-aromatic cyclic group having 4 ring atoms, at least one of which is a heteroatom selected from N, O and S. The term encompasses oxetane and azetidine, which in one embodiment are optionally substituted by hydroxy.

In some cases, heterocyclic rings may be substituted on one or more ring carbon atoms by oxo. Examples of rings of this type include pyridone and pyridazinone. However, it should be noted that although pyridone and pyridazinone are defined herein as a “heterocyclic ring”, any aromatic tautomers of pyridone and pyridazinone are also encompassed, as shown below:

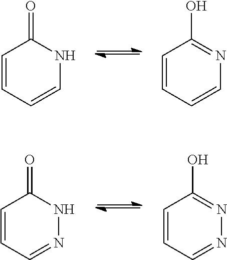 [SMILES: O=c1cccc[nH]1 ; Oc1ccccn1 ; O=c1cccn[nH]1 ; Oc1cccnn1]

The term “5-10 membered heteroaryl” refers to a cyclic group with aromatic character having 5-10 ring atoms, at least one of which is a heteroatom independently selected from N, O and S. The term encompasses pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl and pyrazinyl. Bicyclic heteroaryl compounds are also encompassed such as furo[3,2-b]pyridinyl, pyrazolo[1,5-a]pyridinyl and imidazo[1,2-a]pyridinyl. Where a heteroaryl group contains more than one ring, not all rings must contain a heteroatom, and not all rings must be aromatic in character. Other heteroaryl groups, for example, 5-9 membered heteroaryl, 5-8 membered heteroaryl, 5-7 membered heteroaryl, 5-6 membered heteroaryl, 6-10 membered heteroaryl, 6-9 membered heteroaryl, 6-8 membered heteroaryl, 6-7 membered heteroaryl, 5 membered heteroaryl, 6 membered heteroaryl, 7 membered heteroaryl, 8 membered heteroaryl, 9 membered heteroaryl and 10 membered heteroaryl are as defined above but contain different numbers of ring atoms. Further examples of heteroaryl include indolyl, indazolyl, benzofuranyl, benzimidazolyl, benzothiazolyl, benzothiophenyl, quinolinyl, isoquinolinyl and quinazolinyl.

The term “hydroxy” (which may also be referred to as “hydroxyl”) refers to an —OH group.

The term “C₁₋₆ hydroxyalkyl” (e.g. C₁₋₅ hydroxyalkyl, C₁₋₄ hydroxyalkyl, C₁₋₃ hydroxyalkyl, C₁₋₂ hydroxyalkyl or C₁ hydroxyalkyl) refers to a straight or a branched fully saturated hydrocarbon group containing the specified number of carbon atoms and at least one —OH group. Examples include —CH₂C(H)OH—, —C(H)OHCH₃, —C(H)OH—, —CH₂OH and —CH₂CH₂OH.

The term “C₁₋₆ methoxyalkyl” (e.g. C₁₋₅ methoxyalkyl, C₁₋₄ methoxyalkyl, C₁₋₃ methoxyalkyl, C₁₋₂ methoxyalkyl or C₁ methoxyalkyl) refers to a straight or a branched fully saturated hydrocarbon group containing the specified number of carbon atoms and at least one methoxy (OCH₃) group, for example —CH₂OCH₃, —CH₂CH₂OCH₃ or —C(H)(OCH₃)CH₃.

The term “oxo” refers to a ═O substituent, whereby an oxygen atom is doubly bonded to carbon (e.g. C═O) or another element (e.g. S═O, S(═O)₂). The carbon or other element is suitably an atom of an alkyl, cycloalkyl or heterocyclyl group.

The term “halo” refers to fluorine, chlorine, bromine or iodine. Particular examples of halo are fluorine and bromine, especially fluorine.

For a substituent of the type C₁₋₆ alkyl-NR′R″, the NR′R″ group can be located at any feasible position on the C₁₋₆ group, including for example —CH₂—NR′R″, —CH₂CH₂—NR′R″ and —CH(NR′R″)—CH₃.

Where substituents are indicated as being optionally substituted on an available carbon atom in formula (Ia) in the embodiments and preferences set out below, the optional substituent is attached to an available carbon atom, which means a carbon atom which is attached to a hydrogen atom i.e. a C—H group. The optional substituent replaces the hydrogen atom attached to the carbon atom.

In some embodiments, R^{A} is phenyl optionally substituted on an available carbon atom by one or more R^{1A}.

In some embodiments, R^{A} is phenyl fused to a 5-7 membered heterocyclic ring, and is optionally substituted on an available atom (e.g. a carbon or nitrogen atom) by one or more R^{1A}. Suitably, R^{A} is phenyl fused to a 5-6 membered heterocyclic ring, for example R^{A} is selected from the group consisting of 2,3-dihydrobenzofuranyl, indolinyl, 1,2,3,4-tetrahydroquinolinyl, benzo-1,4-dioxanyl, 1,3-benzodiazole and 3,4-dihydro-2H-1,4-benzoxazine; and in particular is 2,3-dihydrobenzofuranyl, benzo-1,4-dioxanyl or 3,4-dihydro-2H-1,4-benzoxazine.

In some embodiments, R^{A} is 5-10 membered heteroaryl, and is optionally substituted on an available atom (e.g. a carbon or nitrogen atom) by one or more R^{1A}. Suitably, R^{A} is selected from the group consisting of pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl and quinazolinyl, and in particular is pyrazolyl. In one embodiment, R^{A} is pyrazolyl substituted by R^{1A}, wherein R^{1A} is methyl.

In some embodiments, R^{A} is 5-6 membered heteroaryl fused to a 5-7 membered heterocyclic ring, and is optionally substituted on an available atom (e.g. a carbon or nitrogen atom) by one or more R^{1A}. Suitably, R^{A} is 2,3-dihydropyrazolo[5,1-b]oxazolyl, 2,3-dihydrofuro[2,3-b]pyridinyl, 2,3-dihydrofuro[3,2-b]pyridinyl, 2,3-dihydro-[1,4]dioxino[2,3-c]pyridinyl or 2,3-dihydro-[1,4]dioxino[2,3-b]pyridinyl.

In some embodiments, R^{A} is phenyl, phenyl fused to a 5-6 membered heterocyclic ring (such as selected from the group consisting of 2,3-dihydrobenzofuranyl, indolinyl, 1,2,3,4-tetrahydroquinolinyl, benzo-1,4-dioxanyl, 1,3-benzodiazole and 3,4-dihydro-2H-1,4-benzoxazine), 5-6 membered heteroaryl (such as pyrrolyl, pyrazolyl, pyrimidinyl or pyridyl) or 5-6 membered heteroaryl fused to a 5-6 membered heterocyclic ring (such as 2,3-dihydropyrazolo[5,1-b]oxazolyl, 2,3-dihydrofuro[2,3-b]pyridinyl, 2,3-dihydro-[1,4]dioxino[2,3-c]pyridinyl or 2,3-dihydro-[1,4]dioxino[2,3-b]pyridinyl); and in particular is phenyl; and is optionally substituted on an available atom (e.g. a carbon or nitrogen atom) by one or more R^{1A}.

In some embodiments, R^{A} is unsubstituted. In another embodiment, R^{A} is substituted by one or more (such as one, two or three e.g. one) R^{1A}. Suitably, each R^{1A} is independently selected from the group consisting of halo, C₁₋₆ alkyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, NHCOCH₃, CO₂H, CONR^{2A}R^{3A}, C₃₋₆ cycloalkyl, oxo, and a 4 membered heterocyclic ring, wherein the 4 membered heterocyclic ring is optionally substituted by hydroxy; and in particular is independently selected from halo, C₁₋₆ alkyl, C₁₋₆ hydroxyalkyl, C₁₋₆ methoxyalkyl, C₁₋₆ haloalkyl, hydroxy, OC₁₋₆ alkyl, oxo, and oxetane or azetidine either of which is optionally substituted by hydroxy; e.g. C₁₋₆ alkyl or OC₁₋₆ alkyl. In one embodiment, R^{1A} is OCH₃.

In some embodiments, R^{2A} and R^{3A} are independently selected from the group consisting of H and C₁₋₆ alkyl, e.g. C₁₋₄ alkyl. Suitably, R^{2A} and R^{3A} are independently selected from the group consisting of H, methyl and ethyl. In one embodiment, R^{2A} and R^{3A} are both H. In one embodiment, R^{2A} and R^{3A} are both methyl. In one embodiment, one of R^{2A} and R^{3A} is H and the other is methyl.

In some embodiments, R^{2A} and R^{3A} together with the N atom to which they are attached combine to form a 4-7 membered heterocyclic ring (in particular a 5-7 membered heterocyclic ring) which is optionally substituted on an available atom by one or more groups selected from C₁₋₂ alkyl and oxo. In some embodiments, R^{2A} and R^{3A} together with the N atom to which they are attached combine to form a 4 membered heterocyclic ring which is optionally substituted on an available atom by one or more groups selected from C₁₋₂ alkyl and oxo. In some embodiments, R^{2A} and R^{3A} together with the N atom to which they are attached combine to form a 5 membered heterocyclic ring which is optionally substituted on an available atom by one or more groups selected from C₁₋₂ alkyl and oxo. In some embodiments, R^{2A} and R^{3A} together with the N atom to which they are attached combine to form a 6 membered heterocyclic ring which is optionally substituted on an available atom by one or more groups selected from C₁₋₂ alkyl and oxo. In some embodiments, R^{2A} and R^{3A} together with the N atom to which they are attached combine to form a 7 membered heterocyclic ring which is optionally substituted on an available atom by one or more groups selected from C₁₋₂ alkyl and oxo. In some embodiments, R^{2A} and R^{3A} together with the N atom to which they are attached combine to form pyrrolidin-1-yl, piperidin-1-yl, morpholin-4-yl, thiomorpholin-4-yl, 1-oxo-thiomorpholin-4-yl, 1,1-dioxo-thiomorpholin-4-yl, 1,4-piperazin-1-yl or N-methyl-1,4-piperazin-1-yl. In some embodiments, R^{2A} and R^{3A} together with the N atom to which they are attached combine to form pyrrolidin-1-yl or piperidin-1-yl. In some embodiments, R^{2A} and R^{3A} together with the N atom to which they are attached combine to form pyrrolidin-1-yl. In some embodiments, R^{2A} and R^{3A} together with the N atom to which they are attached combine to form piperidin-1-yl.

In some embodiments, R^{B} is phenyl optionally substituted on an available carbon atom by one or more R_{1B}.

In some embodiments, R^{B} is phenyl fused to a 5-7 membered heterocyclic ring, and is optionally substituted on an available atom (e.g. a carbon or nitrogen atom) by one or more R^{1B}. Suitably, R^{B} is phenyl fused to a 5-6 membered heterocyclic ring, for example R^{B} is 2,3-dihydrobenzofuranyl, indolinyl, 1,2,3,4-tetrahydroquinolinyl, benzo-1,4-dioxanyl, 1,3-benzodiazole and 3,4-dihydro-2H-1,4-benzoxazine; and in particular is 2,3-dihydrobenzofuranyl, indolinyl, 1,2,3,4-tetrahydroquinolinyl, benzo-1,4-dioxanyl or 1,3-benzodiazole.

In some embodiments, R^{B} is 5-10 membered heteroaryl, and is optionally substituted on an available atom (e.g. a carbon or nitrogen atom) by one or more R^{1B}. Suitably, R^{B} is selected from the group consisting of pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl and quinazolinyl, and in particular is selected from the group consisting of furanyl, thienyl, pyrazolyl, thiazolyl, isothiazolyl, isoxazolyl, pyridinyl, pyrazinyl, pyrazolo[1,5-a]pyridinyl, imidazo[1,2-a]pyridinyl and indazolyl. In one embodiment, R^{B} is pyrazolyl.

In some embodiments, R^{B} is 5-6 membered heteroaryl fused to a 5-7 membered heterocyclic ring, and is optionally substituted on an available atom (e.g. a carbon or nitrogen atom) by one or more R^{1B}. Suitably, R^{B} is 2,3-dihydropyrazolo[5,1-b]oxazolyl, 2,3-dihydrofuro[2,3-b]pyridinyl, 2,3-dihydrofuro[3,2-b]pyridinyl, 2,3-dihydro-[1,4]dioxino[2,3-c]pyridinyl or 2,3-dihydro-[1,4]dioxino[2,3-b]pyridinyl.

In some embodiments, R^{B} is 5-7 membered heterocyclyl (such as 5-6 membered heterocyclyl), and is optionally substituted on an available atom (e.g. a carbon or nitrogen atom) by one or more R^{1B}. Suitably, R^{B} is selected from the group consisting of pyrrolidinyl, tetrahydrofuranyl, tetrahydrothienyl, tetrahydropyranyl, piperidinyl, piperazinyl, morpholinyl, thiomorpholinyl, pyridone and pyridazinone; such as pyridone or pyridazinone, and in particular is pyridone.

In some embodiments, R^{B} is C₃₋₆ cycloalkyl and is optionally substituted on an available carbon atom by one or more R^{1B}.

In some embodiments, R^{B} is selected from the group consisting of phenyl, phenyl fused to a 5-7 membered heterocyclic ring (such as selected from the group consisting of 2,3-dihydrobenzofuranyl, indolinyl, 1,2,3,4-tetrahydroquinolinyl, benzo-1,4-dioxanyl, 1,3-benzodiazole and 3,4-dihydro-2H-1,4-benzoxazine), pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl, quinazolinyl, pyrrolidinyl, tetrahydrofuranyl, tetrahydrothienyl, tetrahydropyranyl, piperidinyl, piperazinyl, morpholinyl, thiomorpholinyl, pyridone and pyridazinone; and is optionally substituted on an available atom (e.g. a carbon or nitrogen atom) by one or more R^{1B}.

In some embodiments, R^{B} is unsubstituted. In another embodiment, R^{B} is substituted by one or more (such as one, two or three e.g. one) R^{1B}. Suitably, each R^{1B} is independently selected from the group consisting of halo, C₁₋₆ alkyl, C₁₋₆ hydroxyalkyl, C₁₋₆ haloalkyl, hydroxy, OC₁₋₆ alkyl, OC₁₋₆ hydroxyalkyl, OC₁₋₆ haloalkyl, cyano, NR^{2B}R^{3B}, CONR^{2B}R^{3B}, CH₂CONR^{2B}R^{3B} C₃₋₆ cycloalkyl, oxo; and a 4 membered heterocyclic ring, wherein the 4 membered heterocyclic ring is optionally substituted by hydroxy; and in particular is independently selected from the group consisting of fluoro, chloro, methyl, ethyl, CH₂OH, CH₂CH₂OH, CHF₂, hydroxy, OCH₃, OCH₂CH₂OH, OCHF₂, cyano, NH₂, CONH₂, CONHCH₃, CH₂CONH₂, cyclopropyl, oxo, and oxetane or azetidine, either of which is optionally substituted by hydroxy.

In some embodiments, R^{2B} and R^{3B} are independently selected from the group consisting of H and C₁₋₆ alkyl e.g. C₁₋₄ alkyl. Suitably, R^{2B} and R^{3B} are independently selected from the group consisting of H, methyl and ethyl. In one embodiment, R^{2B} and R^{3B} are both H. In one embodiment, R^{2B} and R^{3B} are both methyl. In one embodiment, one of R^{2B} and R^{3B} is H and the other is methyl.

In some embodiments, R^{2B} and R^{3B} together with the N atom to which they are attached combine to form a 4-7 membered heterocyclic ring (in particular a 5-7 membered heterocyclic ring) which is optionally substituted on an available atom by one or more groups selected from C₁₋₂ alkyl and oxo. In some embodiments, R^{2B} and R^{3B} together with the N atom to which they are attached combine to form a 4 membered heterocyclic ring which is optionally substituted on an available atom by one or more groups selected from C₁₋₂ alkyl and oxo. In some embodiments, R^{2B} and R^{3B} together with the N atom to which they are attached combine to form a 5 membered heterocyclic ring which is optionally substituted on an available atom by one or more groups selected from C₁₋₂ alkyl and oxo. In some embodiments, R^{2B} and R^{3B} together with the N atom to which they are attached combine to form a 6 membered heterocyclic ring which is optionally substituted on an available atom by one or more groups selected from C₁₋₂ alkyl and oxo. In some embodiments, R^{2B} and R^{3B} together with the N atom to which they are attached combine to form a 7 membered heterocyclic ring which is optionally substituted on an available atom by one or more groups selected from C₁₋₂ alkyl and oxo. In some embodiments, R^{2B} and R^{3B} together with the N atom to which they are attached combine to form pyrrolidin-1-yl, piperidin-1-yl, morpholin-4-yl, thiomorpholin-4-yl, 1-oxo-thiomorpholin-4-yl, 1,1-dioxo-thiomorpholin-4-yl, 1,4-piperazin-1-yl or N-methyl-1,4-piperazin-1-yl. In some embodiments, R^{2B} and R^{3B} together with the N atom to which they are attached combine to form pyrrolidin-1-yl or piperidin-1-yl. In some embodiments, R^{2B} and R^{3B} together with the N atom to which they are attached combine to form pyrrolidin-1-yl. In some embodiments, R^{2B} and R^{3B} together with the N atom to which they are attached combine to form piperidin-1-yl.

In some embodiments, Y¹, Y² and Y³ are independently selected from the group consisting of CR^{4A} and N, wherein at least two of Y¹, Y² and Y³ are CR^{4A}; wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A}; wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A}, wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo. Suitably, Y¹, Y² and Y³ are all CR^{4A}, wherein suitably R^{4A} is H.

In some embodiments, Y² is absent and one of Y¹ and Y³ is S, O or NR^{5A}, and the remaining Y¹ or Y³ is N or CR^{6A}; wherein R^{5A} is H or C₁₋₃ alkyl; and R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A}; wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A}, wherein R^{14A} and R^{1A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo. Suitably, R^{5A} is H and R^{6A} is H.

In some embodiments, Z¹ is N and Z² is CH. In some embodiments, Z¹ is CH and Z² is N. In some embodiments, Z¹ is CH and Z² is CH. Suitably, Z¹ is N and Z² is CH.

In some embodiments, R^{C} is H, fluoro, CH₃, CH₂OH or CH₂OCH₃. Suitably, R^{C} is H.

In some embodiments, R^{D} is H. In some embodiments, R^{D} is fluoro. Suitably, R^{D} is H.

In some embodiments, R^{C} is fluoro and R^{D} is fluoro. Suitably, R^{C} is H and R^{D} is H.

In some embodiments, m is 0. In some embodiments, m is 1. Suitably, m is 0.

In some embodiments, X is S(═O)₁₋₂, —CH₂S(═O)₁₋₂—, S(═O)(═NH), —S(═O)₂NH—, —NHS(═O)₂—, CH₂, —CH₂O— or CH₂NH—.

In some embodiments, X is S(═O)₁₋₂ or S(═O)(═NH). Suitably, X is S(═O)₁₋₂, in particular S(═O)₂.

It should be noted that when X is —CH₂—S(═O)₁₋₂—, —S(═O)₂NH—, —NH—S(═O)₂—, —CH₂O— or CH₂NH—. it is intended that the left hand group as drawn is directly bonded to group R^{A} and the right hand group as drawn is directly bonded to the bicyclic core structure as shown below for —CH₂—S(═O)₂—:

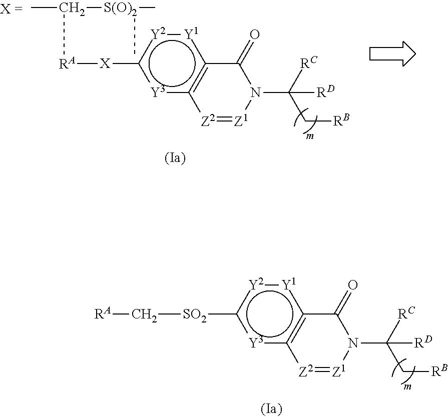

In some embodiments, R^{E} and R^{F} are H. In other embodiments, R^{E} and R^{F} join to form a C₃₋₅ heterocycloalkyl ring such as an oxetanyl ring e.g. 3-oxetanyl.

In one embodiment, the compound of formula (Ia) is a compound of formula (IIa) as follows:

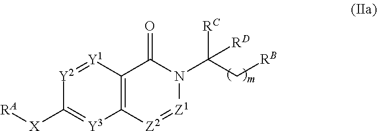

wherein Y¹, Y² and Y³ are independently selected from the group consisting of CR^{4A} and N, wherein at least two of Y¹, Y² and Y³ are CR^{4A};
wherein R^{A}, R^{B}, R^{C}, R^{D}, m, X, Z¹, Z² and R^{4A} are as defined hereinabove;
or a pharmaceutically acceptable salt and/or solvate thereof.

In one embodiment, the compound of formula (Ia) is a compound of formula (IIaA) as follows:

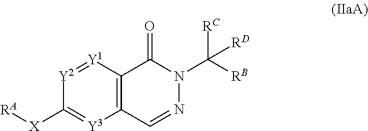

wherein Y¹, Y² and Y³ are independently selected from the group consisting of CR^{4A} and N, wherein at least two of Y¹, Y² and Y³ are CR^{4A};
wherein R^{A}, R^{B}, R^{C}, R^{D}, X and R^{4A} are as defined hereinabove;
or a pharmaceutically acceptable salt and/or solvate thereof.

In one embodiment, the compound of formula (Ia) is a compound of formula (IIaB) as follows:

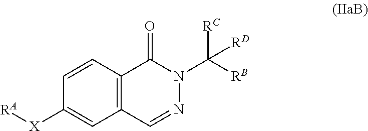

wherein R^{A}, R^{B}, R^{C}, R^{D} and X are as defined hereinabove;
or a pharmaceutically acceptable salt and/or solvate thereof.

In one embodiment, the compound of formula (Ia) is a compound of formula (IIaC) as follows:

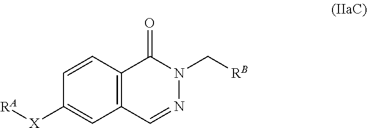

wherein R^{A}, R^{B} and X are as defined hereinabove;
or a pharmaceutically acceptable salt and/or solvate thereof.

In one embodiment, the compound of formula (Ia) is a compound of formula (IIaD) as follows:

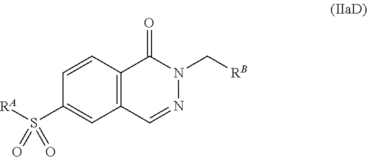

wherein R^{A} and R^{B} are as defined hereinabove;
or a pharmaceutically acceptable salt and/or solvate thereof.

In one embodiment, the compound of formula (Ia) is a compound of formula (IIIa) as follows:

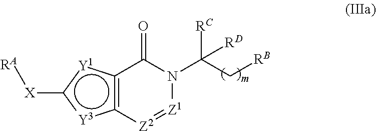

wherein one of Y¹ and Y³ is S, O or NR^{5A}, and the remaining Y¹ or Y³ is N or CR^{6A};
wherein R^{A}, R^{B}, R^{C}, R^{D}, m, X, Z¹, Z², R^{5A} and R^{6A} are as defined hereinabove;
or a pharmaceutically acceptable salt and/or solvate thereof.

In one embodiment, the compound of formula (Ia) is a compound of formula (IIIaA) as follows:

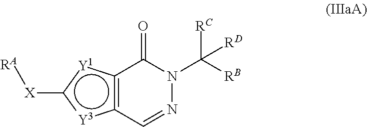

wherein one of Y¹ and Y³ is S, O or NR^{5A}, and the remaining Y¹ or Y³ is N or CR^{6A};
wherein R^{A}, R^{B}, R^{C}, R^{D}, X, R^{5A} and R^{6A} are as defined hereinabove;
or a pharmaceutically acceptable salt and/or solvate thereof.

In one embodiment, the compound of formula (Ia) is a compound of formula (IIIaB) as follows:

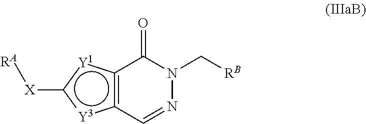

wherein one of Y¹ and Y³ is S, O or NR^{5A}, and the remaining Y¹ or Y³ is N or CR^{6A};
wherein R^{A}, R^{B}, X, R^{5A} and R^{6A} are as defined hereinabove;
or a pharmaceutically acceptable salt and/or solvate thereof.

In one embodiment, the compound of formula (Ia) is a compound of formula (IIIaC) as follows:

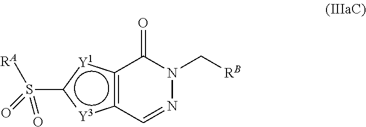

wherein one of Y¹ and Y³ is S, O or NR^{5A}, and the remaining Y¹ or Y³ is N or CR^{6A};
wherein R^{A}, R^{B}, R^{5A} and R^{6A} are as defined hereinabove
or a pharmaceutically acceptable salt and/or solvate thereof.

In one embodiment there is provided a compound of formula (Ia), which is:
2-((1-cyclopropyl-1H-pyrazol-3-yl)methyl)-6-((4-methoxyphenyl)sulfonyl)phthalazin-1(2H)-one;
2-(indolin-4-ylmethyl)-6-((4-methoxyphenyl)sulfonyl)phthalazin-1(2H)-one;
2-((2-hydroxypyridin-4-yl)methyl)-6-((4-methoxyphenyl)sulfonyl)phthalazin-1(2H)-one;
6-((4-methoxyphenyl)sulfonyl)-2-((1-methyl-2-oxo-1,2-dihydropyridin-3-yl)methyl)phthalazin-1(2H)-one;
2-((1-(2-hydroxyethyl)-2-oxo-1,2-dihydropyridin-3-yl)methyl)-6-(4-methoxyphenylsulfonyl)phthalazin-1(2H)-one;
2-((1H-pyrazol-3-yl)methyl)-6-((4-methoxyphenyl)sulfonyl)phthalazin-1(2H)-one;
2-((5-hydroxypyridin-3-yl)methyl)-6-(phenylsulfonyl)phthalazin-1(2H)-one;
2-(2-methoxy-1-(6-methoxypyridin-3-yl)ethyl)-6-(phenylsulfonyl)phthalazin-1(2H)-one;
3-((1-oxo-6-(phenylsulfonyl)phthalazin-2(1H)-yl)methyl)picolinamide;
2-((1-(2-hydroxyethyl)-1H-pyrazol-3-yl)methyl)-6-(phenylsulfonyl)phthalazin-1(2H)-one;
2-((1-ethyl-1H-pyrazol-3-yl)methyl)-6-(phenylsulfonyl)phthalazin-1(2H)-one;
2-((4-methyl-1H-pyrazol-3-yl)methyl)-6-(phenylsulfonyl)phthalazin-1(2H)-one;
3-((1-oxo-6-(phenylsulfonyl)phthalazin-2(1H)-yl)methyl)thiophene-2-carboxamide;
6-methoxy-3-((1-oxo-6-(phenylsulfonyl)phthalazin-2(1H)-yl)methyl)picolinamide;
3-((1-oxo-6-(phenylsulfonyl)phthalazin-2(1H)-yl)methyl)-1H-pyrazole-4-carboxamide;
2-((1-(2-hydroxyethyl)-4-methyl-1H-pyrazol-3-yl)methyl)-6-(phenylsulfonyl)phthalazin-1(2H)-one;
3-((6-((4-methoxyphenyl)sulfonyl)-1-oxophthalazin-2(1H)-yl)methyl)-N-methylfuran-2-carboxamide;
3-((1-oxo-6-(phenylsulfonyl)phthalazin-2(1H)-yl)methyl)-1H-pyrazole-4-carbonitrile;
2-(2-hydroxy-1-phenylethyl)-6-(phenylsulfonyl)phthalazin-1(2H)-one;
2-(2-(hydroxymethyl)benzyl)-6-(phenylsulfonyl)phthalazin-1(2H)-one;
2-((6-(2-hydroxyethoxy)pyridin-3-yl)methyl)-6-(phenylsulfonyl)phthalazin-1(2H)-one;
2-((2-(hydroxymethyl)thiophen-3-yl)methyl)-6-(phenylsulfonyl)phthalazin-1(2H)-one;
3-((1-oxo-6-(phenylsulfonyl)phthalazin-2(1H)-yl)methyl)thiophene-2-carbonitrile;
2-((1-(2-hydroxyethyl)-1H-pyrazol-3-yl)methyl)-6-((4-methoxyphenyl)sulfonyl)phthalazin-1(2H)-one;
2-((5-cyclopropyl-1H-pyrazol-3-yl)methyl)-6-(phenylsulfonyl)phthalazin-1(2H)-one;
2-((4-(hydroxymethyl)-1H-pyrazol-3-yl)methyl)-6-(phenylsulfonyl)phthalazin-1(2H)-one;
2-((2,3-dihydropyrazolo[5,1-b]oxazol-6-yl)methyl)-6-(phenylsulfonyl)phthalazin-1(2H)-one;
2-((1-(difluoromethyl)-1H-pyrazol-3-yl)methyl)-6-(phenylsulfonyl)phthalazin-1(2H)-one;
2-((2,3-dihydrobenzo[b][1,4]dioxin-6-yl)methyl)-6-(phenylsulfonyl)phthalazin-1(2H)-one;
2-((2-methylthiazol-5-yl)methyl)-6-(phenylsulfonyl)phthalazin-1(2H)-one;
2-((5-methoxypyridin-3-yl)methyl)-6-(phenylsulfonyl)phthalazin-1(2H)-one;
2-(2-fluorobenzyl)-6-(phenylsulfonyl)phthalazin-1(2H)-one;
2-(2-fluoro-5-methoxybenzyl)-6-(phenylsulfonyl)phthalazin-1(2H)-one;
2-(imidazo[1,2-a]pyridin-5-ylmethyl)-6-(phenylsulfonyl)phthalazin-1(2H)-one;
2-((2,2-dimethyl-2,3-dihydrobenzofuran-5-yl)methyl)-6-(phenylsulfonyl)phthalazin-1(2H)-one;
2-((1-methyl-1H-indazol-5-yl)methyl)-6-(phenylsulfonyl)phthalazin-1(2H)-one;
2-((6-methoxypyridin-2-yl)methyl)-6-(phenylsulfonyl)phthalazin-1(2H)-one;
2-((6-aminopyridin-2-yl)methyl)-6-(phenylsulfonyl)phthalazin-1(2H)-one;
2-((1-methyl-1H-indazol-6-yl)methyl)-6-(phenylsulfonyl)phthalazin-1(2H)-one;
2-(3-(difluoromethoxy)benzyl)-6-((4-methoxyphenyl)sulfonyl)phthalazin-1(2H)-one;
2-((6-methoxypyridin-3-yl)methyl)-6-(phenylsulfonyl)phthalazin-1(2H)-one;
6-((4-methoxyphenyl)sulfonyl)-2-(pyridin-3-ylmethyl)phthalazin-1(2H)-one;
2-(2-aminobenzyl)-6-((4-methoxyphenyl)sulfonyl)phthalazin-1(2H)-one;
6-((4-methoxyphenyl)sulfonyl)-2-phenethylphthalazin-1(2H)-one;
2-(isoxazol-5-ylmethyl)-6-((4-methoxyphenyl)sulfonyl)phthalazin-1(2H)-one;
2-(4-chloro-2-fluorobenzyl)-6-((4-methoxyphenyl)sulfonyl)phthalazin-1(2H)-one;
2-((2,3-dihydrobenzofuran-5-yl)methyl)-6-((4-methoxyphenyl)sulfonyl)phthalazin-1(2H)-one;
2-((1H-indazol-3-yl)methyl)-6-((4-methoxyphenyl)sulfonyl)phthalazin-1(2H)-one;
2-(4-methoxybenzyl)-6-(phenylsulfonyl)phthalazin-1(2H)-one;
2-benzyl-6-((4-methoxyphenyl)sulfonyl)phthalazin-1(2H)-one;
6-((4-methoxyphenyl)sulfonyl)-2-((6-methoxypyridin-3-yl)methyl)phthalazin-1(2H)-one;
2-(2-fluoro-3-methoxybenzyl)-6-((4-methoxyphenyl)sulfonyl)phthalazin-1(2H)-one;
2-(2,6-difluoro-4-methoxybenzyl)-6-((4-methoxyphenyl)sulfonyl)phthalazin-1(2H)-one;
6-((4-methoxyphenyl)sulfonyl)-2-((5-methyl-1H-pyrazol-3-yl)methyl)phthalazin-1(2H)-one;
2-((2-hydroxypyridin-4-yl)methyl)-6-((4-methoxyphenyl)sulfonyl)phthalazin-1(2H)-one;
2-((2-hydroxypyridin-3-yl)methyl)-6-((4-methoxyphenyl)sulfonyl)phthalazin-1(2H)-one;
2-(benzo[d][1.3]dioxol-5-ylmethyl)-6-(phenylsulfonyl)phthalazin-1(2H)-one;
2-((1H-indazol-6-yi)methyl)-6-(phenylsulfonyl)phthalazin-1(2H)-one;
2-((5-methoxypyridin-2-yl)methyl)-6-(phenylsulfonyl)phthalazin-1(2H)-one;
2-((1-methyl-1H-pyrazol-3-yl)methyl)-6-(phenylsulfonyl)phthalazin-1(2H)-one;
2-(2-((1-oxo-6-(phenylsulfonyl)phthalazin-2(1H)-yl)methyl)phenyl)acetamide;
2-((6-(difluoromethoxy)pyridin-3-yl)methyl)-6-(phenylsulfonyl)phthalazin-1(2H)-one;
2-(2-(2-hydroxyethyl)benzyl)-6-(phenylsulfonyl)phthalazin-1(2H)-one;
2-((2-methyl-2,3-dihydrobenzofuran-5-yl)methyl)-6-(phenylsulfonyl)phthalazin-1(2H)-one;
6-(phenylsulfonyl)-2-(pyridin-3-ylmethyl)phthalazin-1(2H)-one;
2-((1-oxo-6-(phenylsulfonyl)phthalazin-2(1H)-yl)methyl)benzamide;
2-(2-methoxy-1-phenylethyl)-6-(phenylsulfonyl)phthalazin-1(2H)-one;
2-((1H-pyrazol-3-yl)methyl)-6-(phenylsulfonyl)phthalazin-1(2H)-one;
6-(phenylsulfonyl)-2-(1-(pyridin-3-yl)ethyl)phthalazin-1(2H)-one;
2-((1,3-dimethyl-1H-pyrazol-4-yl)methyl)-6-(phenylsulfonyl)phthalazin-1(2H)-one;
2-benzyl-6-(phenylsulfonyl)phthalazin-1(2H)-one;
6-((4-methoxyphenyl)sulfonyl)-2-((1-methyl-1H-pyrazol-3-yl)methyl)phthalazin-1(2H)-one;
2-((1-cyclopropyl-1H-pyrazol-3-yl)methyl)-6-(phenylsulfonyl)phthalazin-1(2H)-one;
2-(1-(6-methoxypyridin-3-yl)ethyl)-6-(phenylsulfonyl)phthalazin-1(2H)-one;
2-((6-methylpyridin-2-yl)methyl)-6-(phenylsulfonyl)phthalazin-1(2H)-one;
2-((4-chloro-1-methyl-1H-pyrazol-3-yl)methyl)-6-(phenylsulfonyl)phthalazin-1(2H)-one;
2-((2-methylthiazol-4-yl)methyl)-6-(phenylsulfonyl)phthalazin-1(2H)-one;
2-(difluoro(pyridin-3-yl)methyl)-6-(phenylsulfonyl)phthalazin-1(2H)-one;
2-(2-methoxybenzyl)-6-(phenylsulfonyl)phthalazin-1(2H)-one;
2-((5-methylisoxazol-3-yl)methyl)-6-(phenylsulfonyl)phthalazin-1(2H)-one;
2-(3-methoxybenzyl)-6-(phenylsulfonyl)phthalazin-1(2H)-one;
2-(2-methoxy-1-phenylethyl)-6-((4-methoxyphenyl)sulfonyl)phthalazin-1(2H)-one;
2-((1-ethyl-1H-pyrazol-5-yl)methyl)-6-(phenylsulfonyl)phthalazin-1(2H)-one;
3-((1-oxo-6-(phenylsulfonyl)phthalazin-2(1H)-yl)methyl)benzamide;
3-((1-oxo-6-(phenylsulfonyl)phthalazin-2(1H)-yl)methyl)furan-2-carboxamide;
5-((1-oxo-6-(phenylsulfonyl)phthalazin-2(1H)-yl)methyl)furan-2-carboxamide;
2-((5-methoxypyrazin-2-yl)methyl)-6-(phenylsulfonyl)phthalazin-1(2H)-one;
2-((2-methoxypyridin-3-yl)methyl)-6-(phenylsulfonyl)phthalazin-1(2H)-one;
2-((1,4-dimethyl-1H-pyrazol-3-yl)methyl)-6-(phenylsulfonyl)phthalazin-1(2H)-one;
2-((1,5-dimethyl-1H-pyrazol-3-yl)methyl)-6-(phenylsulfonyl)phthalazin-1(2H)-one;
2-chloro-6-((1-oxo-6-(phenylsulfonyl)phthalazin-2(1H)-yl)methyl)benzamide;
2-methoxy-6-((1-oxo-6-(phenylsulfonyl)phthalazin-2(1H)-yl)methyl)benzamide;
4-((1-oxo-6-(phenylsulfonyl)phthalazin-2(1H)-yl)methyl)thiazole-2-carboxamide;
5-methyl-4-((1-oxo-6-(phenylsulfonyl)phthalazin-2(1H)-yl)methyl) isoxazole-3-carboxamide;
2-((1-isopropyl-1H-pyrazol-3-yl)methyl)-6-(phenylsulfonyl)phthalazin-1(2H)-one;
2-((5-methyl-1H-pyrazol-3-yl)methyl)-6-(phenylsulfonyl)phthalazin-1(2H)-one;
2-((3-methylisothiazol-5-yi)methyl)-6-(phenylsulfonyl)phthalazin-1(2H)-one;
2-((2-hydroxy-6-methylpyridin-3-yl)methyl)-6-(4-methoxyphenylsulfonyl)phthalazin-1(2H)-one;
2-((2-hydroxy-4-methylpyridin-3-yl)methyl)-6-(4-methoxyphenylsulfonyl)phthalazin-1(2H)-one;
2-((5-fluoro-2-hydroxypyridin-3-yl)methyl)-6-(4-methoxyphenylsulfonyl)phthalazin-1(2H)-one;
2-((2-hydroxy-5-isopropylpyridin-3-yl)methyl)-6-(4-methoxyphenylsulfonyl)phthalazin-1(2H)-one;
2-((4-chloro-2-hydroxypyridin-3-yl)methyl)-6-(4-methoxyphenylsulfonyl)phthalazin-1(2H)-one;
2-((4-fluoro-2-hydroxypyridin-3-yl)methyl)-6-(4-methoxyphenylsulfonyl)phthalazin-1(2H)-one;
6-((4-methoxyphenyl)sulfonyl)-2-((4-methyl-1H-pyrazol-3-yl)methyl)phthalazin-1(2H)-one;
2-((3-hydroxypyridin-4-yl)methyl)-6-(4-methoxyphenylsulfonyl)phthalazin-1(2H)-one;
5-methoxy-2-((6-(4-methoxyphenylsulfonyl)-1-oxophthalazin-2(1H)-yl)methyl)benzamide;
2-((4-hydroxypyridin-3-yl)methyl)-6-(4-methoxyphenylsulfonyl)phthalazin-1(2H)-one;
6-(phenylsulfonyl)-2-(pyrazolo[1,5-a]pyridin-2-ylmethyl)phthalazin-1(2H)-one;
2-((2-hydroxy-6-methoxypyridin-3-yl)methyl)-6-(4-methoxyphenylsulfonyl)phthalazin-1(2H)-one;
2-((4-fluoro-1H-pyrazol-3-yl)methyl)-6-(phenylsulfonyl)phthalazin-1(2H)-one;
2-((5-fluoro-1H-pyrazol-3-yl)methyl)-6-(phenylsulfonyl)phthalazin-1(2H)-one;
6-(4-methoxyphenylsulfonyl)-2-((3-(trifluoromethyl)isoxazol-5-yl)methyl)phthalazin-1(2H)-one;
2-((3-hydroxypyridin-2-yl)methyl)-6-(4-methoxyphenylsulfonyl)phthalazin-1(2H)-one;
2-(3-aminobenzyl)-6-(quinolin-8-ylsulfonyl)phthalazin-1(2H)-one;
2-((6-methoxypyridin-3-yl)methyl)-6-(5-methylpyridin-2-ylsulfonyl)phthalazin-1(2H)-one;
6-(3-methoxyphenylsulfonyl)-2-((6-methoxypyridin-3-yl)methyl)phthalazin-1(2H)-one;
3-(2-((6-methoxypyridin-3-yl)methyl)-1-oxo-1,2-dihydrophthalazin-6-ylsulfonyl)thiophene-2-carboxamide;
4-(2-((6-methoxypyridin-3-yl)methyl)-1-oxo-1,2-dihydrophthalazin-6-ylsulfonyl)thiophene-2-carboxamide;
2-(3-aminobenzyl)-6-(phenylsulfinyl)phthalazine-1(2H)-one;
2-(3-aminobenzyl)-6-(phenylsulfonimidoyl)phthalazin-1(2H)-one;
2-(3-aminobenzyl)-6-(pyridin-3-ylsulfonyl)phthalazin-1(2H)-one;
2-((6-methoxypyridin-3-yl)methyl)-1-oxo-N-phenyl-1,2-dihydrophthalazine-6-sulfonamide;
6-((1H-indazol-4-yl)sulfonyl)-2-((6-methoxypyridin-3-yl)methyl)phthalazin-1(2H)-one;
6-((6-(difluoromethoxy)pyridin-3-yl)sulfonyl)-2-((6-methoxypyridin-3-yl)methyl)phthalazin-1(2H)-one;
3-((6-methoxypyridin-3-yl)methyl)-7-((3-phenyloxetan-3-yl)oxy)pyrido[3,4-d]pyridazin-4(3H)-one;
6-((2,3-dihydrobenzofuran-5-yl)sulfonyl)-2-((6-methoxypyridin-3-yl)methyl)phthalazin-1(2H)-one;
6-(2,3-dihydrobenzofuran-5-sulfonimidoyl)-2-((6-methoxypyridin-3-yl)methyl)phthalazin-1(2H)-one;
(R)-6-(2,3-dihydrobenzofuran-5-sulfonimidoyl)-2-((6-methoxypyridin-3-yl)methyl)phthalazin-1(2H)-one;
(S)-6-(2,3-dihydrobenzofuran-5-sulfonimidoyl)-2-((6-methoxypyridin-3-yl)methyl)phthalazin-1(2H)-one;
6-((2,3-dihydro-[1,4]dioxino[2,3-b]pyridin-7-yl)sulfonyl)-2-((6-methoxypyridin-3-yl)methyl)phthalazin-1(2H)-one;
2-((6-methoxypyridin-3-yl)methyl)-6-(phenylsulfonimidoyl)phthalazin-1(2H)-one;
R)-2-((6-methoxypyridin-3-yl)methyl)-6-(phenylsulfonimidoyl)phthalazin-1(2H)-one;
(S)-2-((6-methoxypyridin-3-yl)methyl)-6-(phenylsulfonimidoyl)phthalazin-1(2H)-one;
6-(4-(difluoromethoxy)phenylsulfonimidoyl)-2-((6-methoxypyridin-3-yl)methyl)phthalazin-1(2H)-one;
(R)-6-(4-(difluoromethoxy)phenylsulfonimidoyl)-2-((6-methoxypyridin-3-yl)methyl)phthalazin-1(2H)-one;
(S)-6-(4-(difluoromethoxy)phenylsulfonimidoyl)-2-((6-methoxypyridin-3-yl)methyl)phthalazin-1(2H)-one;
6-(4-methoxyphenylsulfonimidoyl)-2-((6-methoxypyridin-3-yl)methyl)phthalazin-1(2H)-one;
(R)-6-(4-methoxyphenylsulfonimidoyl)-2-((6-methoxypyridin-3-yl)methyl)phthalazin-1(2H)-one;
(S)-6-(4-methoxyphenylsulfonimidoyl)-2-((6-methoxypyridin-3-yl)methyl)phthalazin-1(2H)-one;
6-((1H-pyrazol-3-yl)sulfonyl)-2-((6-methoxypyridin-3-yl)methyl)phthalazin-1(2H)-one;
2-(3-aminobenzyl)-6-((phenyl)sulfonyl)phthalazin-1(2H)-one;
2-(3-aminobenzyl)-6-((4-methoxyphenyl)sulfonyl)phthalazin-1(2H)-one;
2-(3-aminobenzyl)-6-((2,3-dihydrobenzofuran-5-yl)sulfonyl)phthalazin-1(2H)-one;
2-(3-aminobenzyl)-6-(pyridin-2-ylsulfonyl)phthalazin-1(2H)-one;
2-(3-aminobenzyl)-6-((1-methyl-1H-pyrazol-3-yl)sulfonyl)phthalazin-1(2H)-one;
2-(3-aminobenzyl)-6-((5-methylthiophen-2-yl)sulfonyl)phthalazin-1(2H)-one;
6-((4-fluorophenyl)sulfonyl)-2-((6-methoxypyridin-3-yl)methyl)phthalazin-1(2H)-one;
2-((6-methoxypyridin-3-yl)methyl)-6-(3-methylisothiazol-5-ylsulfonyl)phthalazin-1(2H)-one;
2-(3-aminobenzyl)-6-((6-methoxypyridin-3-yl)sulfonyl)phthalazin-1(2H)-one;
2-((6-methoxypyridin-3-yl)methyl)-6-tosylphthalazin-1(2H)-one;
2-(3-aminobenzyl)-6-((2-methylthiazol-4-yl)sulfonyl)phthalazine-1(2H)-one;
2-(3-aminobenzyl)-6-(benzo[d][1,3]dioxol-5-ylsulfonyl)phthalazin-1(2H)-one;
6-(4-(difluoromethoxy)phenylsulfonyl)-2-((6-methoxypyridin-3-yl)methyl)phthalazin-1(2H)-one;
6-((3-chlorophenyl)sulfonyl)-2-((6-methoxypyridin-3-yl)methyl)phthalazin-1(2H)-one;
6-((4-chlorophenyl)sulfonyl)-2-((6-methoxypyridin-3-yl)methyl)phthalazin-1(2H)-one;
6-((2,2-difluorobenzo[d][1,3]dioxol-5-yl)sulfonyl)-2-((6-methoxypyridin-3-yl)methyl)phthalazin-1(2H)-one;
6-((3-fluorophenyl)sulfonyl)-2-((6-methoxypyridin-3-yl)methyl)phthalazin-1(2H)-one;
2-((6-methoxypyridin-3-yl)methyl)-6-((1-methyl-1H-pyrazol-3-yl)sulfonyl)phthalazin-1(2H)-one;
2-((6-methoxypyridin-3-yl)methyl)-6-(4-methylpyridin-2-ylsulfonyl)phthalazin-1(2H)-one;
6-(6-methoxypyridin-2-ylsulfonyl)-2-((6-methoxypyridin-3-yl)methyl)phthalazin-1(2H)-one;
3-(2-((6-methoxypyridin-3-yl)methyl)-1-oxo-1,2-dihydrophthalazin-6-ylsulfonyl)benzamide;
6-(2-fluorophenylsulfonyl)-2-((6-methoxypyridin-3-yl)methyl)phthalazin-1(2H)-one;
6-(2-chlorophenylsulfonyl)-2-((6-methoxypyridin-3-yl)methyl)phthalazin-1(2H)-one;
6-(2-methoxyphenylsulfonyl)-2-((6-methoxypyridin-3-yl)methyl)phthalazin-1(2H)-one;
6-(4-methoxypyridin-2-ylsulfonyl)-2-((6-methoxypyridin-3-yl)methyl)phthalazin-1(2H)-one;
6-((2,2-dimethyl-2,3-dihydrobenzofuran-5-yl)sulfonyl)-2-((6-methoxypyridin-3-yl)methyl)phthalazin-1(2H)-one;
2-((6-methoxypyridin-3-yl)methyl)-6-((2-methyl-2,3-dihydrobenzofuran-5-yl)sulfonyl)phthalazin-1(2H)-one;
2-((6-methoxypyridin-3-yl)methyl)-6-((1-methyl-1H-pyrazol-4-yl)sulfonyl)phthalazin-1(2H)-one;
2-((6-methoxypyridin-3-yl)methyl)-6-(6-methylpyridin-2-ylsulfonyl)phthalazin-1(2H)-one;
6-(3-(difluoromethoxy)phenylsulfonyl)-2-((6-methoxypyridin-3-yl)methyl)phthalazin-1(2H)-one;
2-((6-methoxypyridin-3-yl)methyl)-6-(5-methylthiophen-2-ylsulfonyl)phthalazin-1(2H)-one;
6-(5-methoxypyridin-2-ylsulfonyl)-2-((6-methoxypyridin-3-yl)methyl)phthalazin-1(2H)-one;
6-(2-(difluoromethyl)thiazol-5-ylsulfonyl)-2-((6-methoxypyridin-3-yl)methyl)phthalazin-1(2H)-one;
2-((6-methoxypyridin-3-yl)methyl)-6-(2-methylthiazol-4-ylsulfonyl)phthalazin-1(2H)-one;
2-((6-methoxypyridin-3-yl)methyl)-6-(6-methoxypyridin-3-ylsulfonyl)phthalazin-1(2H)-one;
2-((6-methoxypyridin-3-yl)methyl)-6-(2-methylbenzo[d]thiazol-4-ylsulfonyl)phthalazin-1(2H)-one;
6-(5-methoxypyrazin-2-ylsulfonyl)-2-((6-methoxypyridin-3-yl)methyl)phthalazin-1(2H)-one;
6-(imidazo[1,2-a]pyridin-5-ylsulfonyl)-2-((6-methoxypyridin-3-yl)methyl)phthalazin-1(2H)-one;
6-(1-(difluoromethyl)-1H-pyrazol-3-ylsulfonyl)-2-((6-methoxypyridin-3-yl)methyl)phthalazin-1(2H)-one;
(S)-6-(2,3-dihydrobenzofuran-5-sulfonimidoyl)-2-((6-methoxypyridin-3-yl)methyl)phthalazin-1(2H)-one;
6-((2,3-dihydrobenzo[b][1,4]dioxin-6-yl)sulfonyl)-2-((6-methoxypyridin-3-yl)methyl)phthalazin-1(2H)-one;
6-((2,3-dihydrofuro[2,3-b]pyridin-5-yl)sulfonyl)-2-((6-methoxypyridin-3-yl)methyl)phthalazin-1(2H)-one;
6-(imidazo[1,2-a]pyridin-7-ylsulfonyl)-2-((6-methoxypyridin-3-yl)methyl)phthalazin-1(2H)-one;
6-(1,2-dimethyl-1H-imidazol-4-ylsulfonyl)-2-((6-methoxypyridin-3-yl)methyl)phthalazin-1(2H)-one;
6-(1,5-dimethyl-1H-pyrazol-4-ylsulfonyl)-2-((6-methoxypyridin-3-yl)methyl)phthalazin-1(2H)-one;
(S)-6-(4-methoxyphenylsulfonimidoyl)-2-((6-methoxypyridin-3-yl)methyl)phthalazin-1(2H)-one;
6-(1-cyclopropyl-1H-pyrazol-4-ylsulfonyl)-2-((2-hydroxypyridin-3-yl)methyl)phthalazin-1(2H)-one;
6-(1-cyclopropyl-1H-pyrazol-4-ylsulfonyl)-2-((1-methyl-1H-pyrazol-3-yl)methyl)phthalazin-1(2H)-one;
2-((1H-pyrazol-3-yl)methyl)-6-(benzofuran-5-ylsulfonyl)phthalazin-1(2H)-one;
2-((1-cyclopropyl-1H-pyrazol-3-yl)methyl)-6-((1-cyclopropyl-1H-pyrazol-4-yl)sulfonyl)phthalazin-1(2H)-one;
6-((1H-pyrazol-4-yl)sulfonyl)-2-((6-methoxypyridin-3-yl)methyl)phthalazin-1(2H)-one;
2-(3-(difluoromethoxy)benzyl)-6-((1-methyl-1H-pyrazol-3-yl)sulfonyl)phthalazin-1(2H)-one;
2-(2-fluoro-5-methoxybenzyl)-6-((1-methyl-1H-pyrazol-3-yl)sulfonyl)phthalazin-1(2H)-one;
2-((2-hydroxypyridin-3-yl)methyl)-6-((1-methyl-1H-pyrazol-4-yl)sulfonyl)phthalazin-1(2H)-one;
6-((3,4-dihydro-2H-benzo[b][1,4]oxazin-6-yl)sulfonyl)-2-((1-methyl-1H-pyrazol-3-yl)methyl)phthalazin-1(2H)-one;
6-((3,4-dihydro-2H-benzo[b][1,4]oxazin-6-yl)sulfonyl)-2-((1-(2-hydroxyethyl)-1H-pyrazol-3-yl)methyl)phthalazin-1(2H)-one;
2-((1H-pyrazol-3-yl)methyl)-6-((2,3-dihydrobenzofuran-5-yl)sulfonyl)phthalazin-1(2H)-one;
6-((1-methyl-1H-pyrazol-4-yl)sulfonyl)-2-((6-methylpyridin-2-yl)methyl)phthalazin-1(2H)-one;
2-((1-cyclopropyl-1H-pyrazol-3-yl)methyl)-6-((6-methoxypyridin-3-yl)sulfonyl)phthalazin-1(2H)-one;
6-((2,3-dihydrobenzo[b][1,4]dioxin-6-yl)sulfonyl)-2-((1-(2-hydroxyethyl)-1H-pyrazol-3-yl)methyl)phthalazin-1(2H)-one;
2-((1H-pyrazol-3-yl)methyl)-6-((2,3-dihydrobenzo[b][1,4]dioxin-6-yl)sulfonyl)phthalazine-1(2H)-one;
6-((2,3-dihydrobenzofuran-5-yl)sulfonyl)-2-((4-methyl-1H-pyrazol-3-yl)methyl)phthalazin-1(2H)-one;
6-((2,3-dihydrobenzofuran-5-yl)sulfonyl)-2-((5-methyl-1H-pyrazol-3-yl)methyl)phthalazin-1(2H)-one;
2-((6-(1-methyl-1H-pyrazol-3-ylsulfonyl)-1-oxophthalazin-2(1H)-yl)methyl)benzamide;
2-((1H-pyrazol-3-yl)methyl)-6-(pyridin-2-ylsulfonyl)phthalazin-1(2H)-one;
6-((4-(difluoromethoxy)phenyl)sulfonyl)-2-((1-methyl-1H-pyrazol-3-yl)methyl)phthalazin-1(2H)-one;
2-((1-methyl-1H-pyrazol-3-yl)methyl)-6-((2-methyl-2,3-dihydrobenzofuran-5-yl)sulfonyl)phthalazin-1(2H)-one;
2-((1-methyl-1H-pyrazol-3-yl)methyl)-6-((2-methylthiazol-4-yl)sulfonyl)phthalazin-1(2H)-one;
6-((2,3-dihydrobenzo[b][1,4]dioxin-6-yl)sulfonyl)-2-((1-methyl-1H-pyrazol-3-yl)methyl)phthalazin-1(2H)-one;
6-((4-ethoxyphenyl)sulfonyl)-2-((1-methyl-1H-pyrazol-3-yl)methyl)phthalazin-1(2H)-one;
(R)-2-((1-methyl-1H-pyrazol-3-yl)methyl)-6-((2-methyl-2,3-dihydrobenzofuran-5-yl)sulfonyl)phthalazin-1(2H)-one;
2-((1-methyl-1H-pyrazol-3-yl)methyl)-6-((4-(trifluoromethoxy)phenyl)sulfonyl)phthalazin-1(2H)-one;
(S)-2-((1-methyl-1H-pyrazol-3-yl)methyl)-6-((2-methyl-2,3-dihydrobenzofuran-5-yl)sulfonyl)phthalazin-1(2H)-one;
2-((1H-pyrazol-3-yl)methyl)-6-((3-methylbenzofuran-5-yl)sulfonyl)phthalazin-1(2H)-one;
2-((1H-pyrazol-3-yl)methyl)-6-((3,4-dihydro-2H-benzo[b][1,4]oxazin-6-yl)sulfonyl)phthalazin-1(2H)-one;
2-((1H-pyrazol-3-yl)methyl)-6-(benzofuran-6-ylsulfonyl)phthalazin-1(2H)-one;
2-((1H-pyrazol-3-yl)methyl)-6-(furo[3,2-b]pyridin-5-ylsulfonyl)phthalazin-1(2H)-one;
2-((1H-pyrazol-3-yl)methyl)-6-((2-methylbenzofuran-5-yl)sulfonyl)phthalazin-1(2H)-one;
2-((1H-pyrazol-3-yl)methyl)-6-(furo[2,3-b]pyridin-5-ylsulfonyl)phthalazin-1(2H)-one;
6-((1H-indol-5-yl)sulfonyl)-2-((1H-pyrazol-3-yl)methyl)phthalazin-1(2H)-one;
2-((1H-pyrazol-3-yl)methyl)-6-(benzo[b]thiophen-5-ylsulfonyl)phthalazin-1(2H)-one;
2-((1H-pyrazol-3-yl)methyl)-6-((1-cyclopropyl-1H-pyrazol-4-yl)sulfonyl)phthalazin-1(2H)-one;
2-((1H-pyrazol-3-yl)methyl)-6-((2,3-dihydro-[1,4]dioxino[2,3-b]pyridin-6-yl)sulfonyl)phthalazin-1(2H)-one;
2-((1H-pyrazol-3-yl)methyl)-6-((2,3-dihydro-[1,4]dioxino[2,3-c]pyridin-7-yl)sulfonyl)phthalazin-1(2H)-one;
2-((1H-pyrazol-3-yl)methyl)-6-((1-methyl-1H-indol-5-yl)sulfonyl)phthalazin-1(2H)-one;
6-((1H-pyrazol-4-yl)sulfonyl)-2-((1-cyclopropyl-1H-pyrazol-3-yl)methyl)phthalazin-1(2H)-one;
2-((1H-pyrazol-3-yl)methyl)-6-(pyridin-3-ylsulfonyl)phthalazin-1(2H)-one;
2-((1H-pyrazol-3-yl)methyl)-6-((2,3-dihydro-[1,4]dioxino[2,3-b]pyridin-7-yl)sulfonyl)phthalazin-1(2H)-one;
2-((1H-pyrazol-3-yl)methyl)-6-((6-methoxypyridin-3-yl)sulfonyl)phthalazin-1(2H)-one;
2-((1-cyclopropyl-1H-pyrazol-3-yl)methyl)-6-((1-methyl-1H-pyrazol-4-yl)sulfonyl)phthalazin-1(2H)-one;
6-(1-(2-hydroxyethyl)-1H-pyrazol-3-ylsulfonyl)-2-((2-hydroxypyridin-3-yl)methyl)phthalazin-1(2H)-one;
6-((1H-pyrazol-4-yl)sulfonyl)-2-((6-methylpyridin-2-yl)methyl)phthalazin-1(2H)-one;
6-((1H-pyrazol-3-yl)sulfonyl)-2-((6-methylpyridin-2-yl)methyl)phthalazin-1(2H)-one;
2-((1H-pyrazol-3-yl)methyl)-6-((1-methyl-1H-pyrazol-4-yl)sulfonyl)phthalazin-1(2H)-one;
2-((1H-pyrazol-3-yl)methyl)-6-((1-(2-hydroxyethyl)-1H-pyrazol-4-yl)sulfonyl)phthalazin-1(2H)-one;
7-fluoro-2-((1-methyl-1H-pyrazol-3-yl)methyl)-6-(phenylsulfonyl)phthalazin-1(2H)-one;
5-fluoro-2-((1-methyl-1H-pyrazol-3-yl)methyl)-6-(phenylsulfonyl)phthalazin-1(2H)-one;
7-((2-hydroxyethyl)amino)-2-((1-methyl-1H-pyrazol-3-yl)methyl)-6-(phenylsulfonyl)phthalazin-1(2H)-one;
8-fluoro-2-((1-methyl-1H-pyrazol-3-yl)methyl)-6-(phenylsulfonyl)phthalazin-1(2H)-one;
7-((2-hydroxyethyl)amino)-2-((2-hydroxypyridin-3-yl)methyl)-6-(phenylsulfonyl)phthalazin-1(2H)-one;
5-((2-hydroxyethyl)amino)-2-((2-hydroxypyridin-3-yl)methyl)-6-(phenylsulfonyl)phthalazin-1(2H)-one;
3-((1-methyl-1H-pyrazol-3-yl)methyl)-7-(phenylsulfonyl)pyrido[3,4-d]pyridazin-4(3H)-one;
3-((1-methyl-1H-pyrazol-3-yl)methyl)-7-(phenylsulfinyl)pyrido[3,4-d]pyridazin-4(3H)-one;
7-((1-methyl-1H-pyrazol-3-yl)methyl)-3-(phenylsulfonyl)pyrido[2,3-d]pyridazin-8(7H)-one;
6-((1H-pyrazol-4-yl)sulfonyl)-2-((5-fluoro-6-methylpyridin-2-yl)methyl)phthalazin-1(2H)-one;
6-((1H-pyrazol-4-yl)sulfonyl)-2-((3-fluoro-6-methylpyridin-2-yl)methyl)phthalazin-1(2H)-one;
6-(1H-pyrazol-4-ylsulfonyl)-2-((2,3-dihydrofuro[3,2-b]pyridin-5-yl)methyl)phthalazin-1(2H)-one;
2-((1H-pyrazol-3-yl)methyl)-6-(pyridin-4-ylsulfonyl)phthalazin-1(2H)-one;
2-((2,4-dihydroxypyridin-3-yl)methyl)-6-(4-methoxyphenylsulfonyl)phthalazin-1(2H)-one;
2-((1-cyclopropyl-1H-pyrazol-3-yl)methyl)-6-(furo[3,2-b]pyridin-5-ylsulfonyl)phthalazin-1(2H)-one;
2-((1H-pyrazol-3-yl)methyl)-6-((5-methoxypyridin-2-yl)sulfonyl)phthalazin-1(2H)-one;
2-((1H-pyrazol-3-yl)methyl)-6-((2,3-dihydrofuro[2,3-c]pyridin-5-yl)sulfonyl)phthalazin-1(2H)-one;
6-((1-cyclopropyl-1H-pyrazol-4-yl)sulfonyl)-2-((6-methylpyridin-2-yl)methyl)phthalazin-1(2H)-one;
2-((1H-pyrazol-3-yl)methyl)-6-(pyrazin-2-ylsulfonyl)phthalazin-1(2H)-one;
2-(imidazo[1,2-a]pyridin-3-ylmethyl)-6-(phenylsulfonyl)phthalazin-1(2H)-one;
2-((1-methyl-1H-pyrazol-3-yl)methyl)-6-(phenylsulfonimidoyl)phthalazin-1(2H)-one;
2-((1H-pyrazol-3-yl)methyl)-7-((2-hydroxyethyl)amino)-6-(phenylsulfonyl)phthalazin-1(2H)-one;
6-((1-(2-hydroxyethyl)-1H-pyrazol-4-yl)sulfonyl)-2-((1-methyl-1H-pyrazol-3-yl)methyl)phthalazin-1(2H)-one;
2-((1-cyclopropyl-1H-pyrazol-3-yl)methyl)-7-((2-hydroxyethyl)amino)-6-(phenylsulfonyl)phthalazin-1(2H)-one;
3-((1H-pyrazol-3-yl)methyl)-7-((1-methyl-1H-pyrazol-4-yl)sulfonyl)pyrido[3,4-d]pyridazin-4(3H)-one;
7-((1H-pyrazol-3-yl)methyl)-3-((1-methyl-1H-pyrazol-4-yl)sulfonyl)pyrido[2,3-d]pyridazin-8(7H)-one;
2-((1H-pyrazol-3-yl)methyl)-7-((2-hydroxyethyl)amino)-6-((1-methyl-1H-pyrazol-4-yl)sulfonyl)phthalazin-1(2H)-one;
2-((1H-pyrazol-3-yl)methyl)-6-((1-(2-hydroxyethyl)-1H-pyrazol-4-yl)sulfonyl)phthalazin-1(2H)-one;
2-((6-methylpyridin-2-yl)methyl)-6-((1-(oxetan-3-yl)-1H-pyrazol-4-yl)sulfonyl)phthalazin-1(2H)-one;
6-((1-(2-hydroxyethyl)-1H-pyrazol-4-yl)sulfonyl)-2-((6-methoxypyridin-3-yl)methyl)phthalazin-1(2H)-one;
2-((1-cyclopropyl-1H-pyrazol-3-yl)methyl)-6-((1-(2-hydroxyethyl)-1H-pyrazol-4-yl)sulfonyl)phthalazin-1(2H)-one;
2-((1-(oxetan-3-yl)-1H-pyrazol-3-yl)methyl)-6-(phenylsulfonyl)phthalazin-1(2H)-one;
2-((6-(1-hydroxyethyl)pyridin-2-yl)methyl)-6-((1-methyl-1H-pyrazol-4-yl)sulfonyl)phthalazin-1(2H)-one;
2-((6-cyclopropylpyridin-2-yl)methyl)-6-((1-methyl-1H-pyrazol-4-yl)sulfonyl)phthalazin-1(2H)-one;
6-((1-(azetidin-3-yl)-1H-pyrazol-4-yl)sulfonyl)-2-((6-methylpyridin-2-yl)methyl)phthalazin-1(2H)-one;
6-((1-(2-aminoethyl)-1H-pyrazol-4-yl)sulfonyl)-2-((6-methylpyridin-2-yl)methyl)phthalazin-1(2H)-one;
(S)-2-((6-(1-hydroxyethyl)pyridin-2-yl)methyl)-6-((1-methyl-1H-pyrazol-4-yl)sulfonyl)phthalazin-1(2H)-one;
(R)-2-((6-(1-hydroxyethyl)pyridin-2-yl)methyl)-6-((1-methyl-1H-pyrazol-4-yl)sulfonyl)phthalazin-1(2H)-one;
6-((1-(2-hydroxyethyl)-1H-pyrazol-3-yl)sulfonyl)-2-((6-methoxypyridin-3-yl)methyl)phthalazin-1(2H)-one;
2-((1H-pyrazol-3-yl)methyl)-6-((1-(2-methoxyethyl)-1H-pyrazol-4-yl)sulfonyl)phthalazin-1(2H)-one;
6-((2-(1-hydroxyethyl)thiazol-4-yl)sulfonyl)-2-((6-methylpyridin-2-yl)methyl)phthalazin-1(2H)-one;
2-((6-(3-hydroxyoxetan-3-yl)pyridin-2-yl)methyl)-6-((1-methyl-1H-pyrazol-4-yl)sulfonyl)phthalazin-1(2H)-one;
6-((1H-pyrazol-4-yl)sulfonyl)-2-((2-methoxypyridin-3-yl)methyl)phthalazin-1(2H)-one;
2-((1-(2-hydroxyethyl)-1H-pyrazol-3-yl)methyl)-6-((6-methoxypyridin-3-yl)sulfonyl)phthalazin-1(2H)-one;
2-((1H-pyrazol-3-yl)methyl)-6-((6-methoxypyridin-2-yl)sulfonyl)phthalazin-1(2H)-one;
6-((1H-pyrazol-4-yl)sulfonyl)-2-((6-cyclopropylpyridin-2-yl)methyl)phthalazin-1(2H)-one;
2-((1H-pyrazol-3-yl)methyl)-6-((6-methylpyridin-2-yl)sulfonyl)phthalazin-1(2H)-one;
2-((1H-pyrazol-3-yl)methyl)-6-((5-methylpyridin-2-yl)sulfonyl)phthalazin-1(2H)-one;
2-((1H-pyrazol-3-yl)methyl)-6-((6-methylpyridin-3-yl)sulfonyl)phthalazin-1(2H)-one;
6-(1H-pyrazol-4-ylsulfonyl)-2-((5-methoxy-6-methylpyridin-2-yl)methyl)phthalazin-1(2H)-one;
2-((1-(2-hydroxyethyl)-1H-pyrazol-3-yl)methyl)-6-((5-methoxypyridin-2-yl)sulfonyl)phthalazin-1(2H)-one;
6-(1H-pyrazol-4-ylsulfonyl)-2-((2,3-dihydrofuro[2,3-b]pyridin-5-yl)methyl)phthalazin-1(2H)-one;
6-((1H-pyrazol-4-yl)sulfonyl)-2-((6-methoxypyridin-2-yl)methyl)phthalazin-1(2H)-one;
6-((1H-pyrazol-4-yl)sulfonyl)-2-((6-methylpyridin-3-yl)methyl)phthalazin-1(2H)-one;
(R)-6-((2-(1-hydroxyethyl)thiazol-4-yl)sulfonyl)-2-((6-methylpyridin-2-yl)methyl)phthalazin-1(2H)-one; and
(S)-6-((2-(1-hydroxyethyl)thiazol-4-yl)sulfonyl)-2-((6-methylpyridin-2-yl)methyl)phthalazin-1(2H)-one;
or a pharmaceutically acceptable salt and/or solvate thereof.

In another embodiment there is provided a compound of formula (Ia), which is:
6-((1H-pyrazol-4-yl)sulfonyl)-2-(furo[3,2-b]pyridin-5-ylmethyl)phthalazin-1(2H)-one;
6-((1H-pyrazol-3-yl)sulfonyl)-2-((2,3-dihydrofuro[3,2-b]pyridin-5-yl)methyl)phthalazin-1(2H)-one;
7-((1H-pyrazol-4-yl)sulfonyl)-3-((5-methyl-1H-pyrazol-3-yl)methyl)pyrido[3,4-d]pyridazin-4(3H)-one;
6-((1H-pyrazol-5-yl)sulfonyl)-2-((3-fluoro-6-methylpyridin-2-yl)methyl)phthalazin-1(2H)-one;
6-((1H-pyrazol-5-yl)sulfonyl)-2-((6-cyclopropylpyridin-2-yl)methyl)phthalazin-1(2H)-one;
6-((1H-pyrazol-5-yl)sulfonyl)-2-((5-fluoro-6-methylpyridin-2-yl)methyl)phthalazin-1(2H)-one;
6-((1H-pyrazol-4-yl)sulfonyl)-2-((5-methoxypyridin-2-yl)methyl)phthalazin-1(2H)-one;
2-((1H-pyrazol-3-yl)methyl)-6-((1-(difluoromethyl)-1H-pyrazol-4-yl)sulfonyl)phthalazin-1(2H)-one;
2-((1H-pyrazol-3-yl)methyl)-6-((1-methyl-1H-pyrazol-3-yl)sulfonyl)phthalazin-1(2H)-one;
2-((6-cyclopropylpyridin-2-yl)methyl)-6-((1-(2-hydroxyethyl)-1H-pyrazol-3-yl)sulfonyl)phthalazin-1(2H)-one;
6-((1H-pyrazol-4-yl)sulfonyl)-2-((4-chloro-1-methyl-1H-pyrazol-3-yl)methyl)phthalazin-1(2H)-one;
2-((2,3-dihydrofuro[3,2-b]pyridin-5-yl)methyl)-6-((1-(2-hydroxyethyl)-1H-pyrazol-4-yl)sulfonyl)phthalazin-1(2H)-one;
6-((1H-pyrazol-4-yl)sulfonyl)-2-((1,5-dimethyl-1H-pyrazol-3-yl)methyl)phthalazin-1(2H)-one;
6-((1-(2-hydroxyethyl)-1H-pyrazol-4-yl)sulfonyl)-2-((5-methoxypyridin-2-yl)methyl)phthalazin-1(2H)-one;
2-((5-methyl-1H-pyrazol-3-yl)methyl)-6-((1-methyl-1H-pyrazol-4-yl)sulfonyl)phthalazin-1(2H)-one;
2-((4-methyl-1H-pyrazol-3-yl)methyl)-6-((1-methyl-1H-pyrazol-4-yl)sulfonyl)phthalazin-1(2H)-one;
6-((1H-pyrazol-4-yl)sulfonyl)-2-((4-chloro-5-methyl-1H-pyrazol-3-yl)methyl)phthalazin-1(2H)-one;
6-((1-(difluoromethyl)-1H-pyrazol-4-yl)sulfonyl)-2-((5-methyl-1H-pyrazol-3-yl)methyl)phthalazin-1(2H)-one;
6-((4-fluoro-1H-pyrazol-3-yl)sulfonyl)-2-((6-methylpyridin-2-yl)methyl)phthalazin-1(2H)-one;
2-((5-chloro-1H-pyrazol-3-yl)methyl)-6-((1-methyl-1H-pyrazol-4-yl)sulfonyl)phthalazin-1(2H)-one;
6-((3-chloro-1H-pyrazol-4-yl)sulfonyl)-2-((6-methylpyridin-2-yl)methyl)phthalazin-1(2H)-one;
6-((4-chloro-1H-pyrazol-3-yl)sulfonyl)-2-((6-methylpyridin-2-yl)methyl)phthalazin-1(2H)-one;
2-((1H-pyrazol-3-yl)methyl)-6-((2-methylthiazol-4-yl)sulfonyl)phthalazin-1(2H)-one;
6-((1H-pyrazol-4-yl)sulfonyl)-2-((3-fluoro-5,6-dimethylpyridin-2-yl)methyl)phthalazin-1(2H)-one;
2-((5-fluoro-1H-pyrazol-3-yl)methyl)-6-((1-methyl-1H-pyrazol-4-yl)sulfonyl)phthalazin-1(2H)-one;
6-((1H-pyrazol-4-yl)sulfonyl)-2-((2,3-dihydropyrazolo[5,1-b]oxazol-6-yl)methyl)phthalazin-1(2H)-one;
2-((2,3-dihydropyrazolo[5,1-b]oxazol-6-yl)methyl)-6-((1-methyl-1H-pyrazol-4-yl)sulfonyl)phthalazin-1(2H)-one;
6-((1H-pyrazol-3-yl)sulfonyl)-2-((5-methoxypyridin-2-yl)methyl)phthalazin-1(2H)-one;
6-((1H-pyrazol-4-yl)sulfonyl)-2-((3-fluoro-6-methoxypyridin-2-yl)methyl)phthalazin-1(2H)-one;
2-((2,3-dihydrofuro[3,2-b]pyridin-5-yl)methyl)-6-((2-(1-hydroxyethyl)thiazol-4-yl)sulfonyl)phthalazin-1(2H)-one;
6-((1H-pyrazol-3-yl)sulfonyl)-2-((1-(difluoromethyl)-1H-pyrazol-3-yl)methyl)phthalazin-1(2H)-one;
6-((1H-pyrazol-3-yl)sulfonyl)-2-((3-fluoro-6-methoxypyridin-2-yl)methyl)phthalazin-1(2H)-one;
2-(furo[3,2-b]pyridin-5-ylmethyl)-6-((1-methyl-1H-pyrazol-4-yl)sulfonyl)phthalazin-1(2H)-one;
3-((5-methyl-1H-pyrazol-3-yl)methyl)-7-((1-methyl-1H-pyrazol-4-yl)sulfonyl)pyrido[3,4-d]pyridazin-4(3H)-one;
6-((1H-pyrazol-3-yl)sulfonyl)-2-((4-chloro-5-methyl-1H-pyrazol-3-yl)methyl)phthalazin-1(2H)-one;
2-((2,3-dihydrofuro[3,2-b]pyridin-5-yl)methyl)-6-((1-(2-methoxyethyl)-1H-pyrazol-4-yl)sulfonyl)phthalazin-1(2H)-one;
2-((2,3-dihydrofuro[3,2-b]pyridin-5-yl)methyl)-6-((1-(oxetan-3-yl)-1H-pyrazol-4-yl)sulfonyl)phthalazin-1(2H)-one;
6-((1H-pyrazol-3-yl)sulfonyl)-2-(furo[3,2-b]pyridin-5-ylmethyl)phthalazin-1(2H)-one;
2-(furo[3,2-b]pyridin-5-ylmethyl)-6-((1-methyl-1H-pyrazol-3-yl)sulfonyl)phthalazin-1(2H)-one;
2-((4-chloro-5-methyl-1H-pyrazol-3-yl)methyl)-6-((1-methyl-1H-pyrazol-3-yl)sulfonyl)phthalazin-1(2H)-one;
6-((1H-pyrazol-4-yl)sulfonyl)-2-((4-chloro-1,5-dimethyl-1H-pyrazol-3-yl)methyl)phthalazin-1(2H)-one;
6-((1H-pyrazol-5-yl)sulfonyl)-2-((1-cyclopropyl-1H-pyrazol-3-yl)methyl)phthalazin-1(2H)-one;
6-((1-(difluoromethyl)-1H-pyrazol-4-yl)sulfonyl)-2-((2,3-dihydrofuro[3,2-b]pyridin-5-yl)methyl)phthalazin-1(2H)-one;
2-((2,3-dihydrofuro[3,2-b]pyridin-5-yl)methyl)-6-(1H-pyrazole-4-sulfonimidoyl)phthalazin-1(2H)-one;
6-((1H-pyrazol-4-yl)sulfonyl)-2-((3,5-difluoro-6-methylpyridin-2-yl)methyl)phthalazin-1(2H)-one;
7-((1-(difluoromethyl)-1H-pyrazol-4-yl)sulfonyl)-3-((5-methyl-1H-pyrazol-3-yl)methyl)pyrido[3,4-d]pyridazin-4(3H)-one;
6-((1H-pyrazol-4-yl)sulfonyl)-2-((2,3-dihydro-[1,4]dioxino[2,3-b]pyridin-6-yl)methyl)phthalazin-1(2H)-one;
6-((1H-pyrazol-4-yl)sulfonyl)-2-((2-methylfuro[3,2-b]pyridin-5-yl)methyl)phthalazin-1(2H)-one;
2-((1H-pyrazol-3-yl)methyl)-6-((2-cyclopropylthiazol-4-yl)sulfonyl)phthalazin-1(2H)-one;
7-((1H-pyrazol-4-yl)sulfonyl)-3-((2,3-dihydrofuro[3,2-b]pyridin-5-yl)methyl)pyrido[3,4-d]pyridazin-4(3H)-one;
2-((4,5-dimethyl-1H-pyrazol-3-yl)methyl)-6-((1-methyl-1H-pyrazol-4-yl)sulfonyl)phthalazin-1(2H)-one;
2-((4-chloro-5-methyl-1H-pyrazol-3-yl)methyl)-6-((1-(difluoromethyl)-1H-pyrazol-4-yl)sulfonyl)phthalazin-1(2H)-one;
2-((2,3-dihydrofuro[3,2-b]pyridin-5-yl)methyl)-6-((2-(1-methoxyethyl)thiazol-4-yl)sulfonyl)phthalazin-1(2H)-one;
2-((1H-pyrazol-3-yl)methyl)-6-((2-(1-methoxyethyl)thiazol-4-yl)sulfonyl)phthalazin-1(2H)-one;
2-(furo[3,2-b]pyridin-5-ylmethyl)-6-((2-(1-methoxyethyl)thiazol-4-yl)sulfonyl)phthalazin-1(2H)-one;
2-((3-fluoro-5,6-dimethylpyridin-2-yl)methyl)-6-((2-(1-methoxyethyl)thiazol-4-yl)sulfonyl)phthalazin-1(2H)-one;
6-((1H-pyrazol-4-yl)sulfonyl)-2-((3,4-dihydro-2H-pyrido[3,2-b][1,4]oxazin-6-yl)methyl)phthalazin-1(2H)-one;
6-((1-methyl-1H-pyrazol-4-yl)sulfonyl)-2-((7-methyl-2,3-dihydropyrazolo[5,1-b]oxazol-6-yl)methyl)phthalazin-1(2H)-one;
2-((2,3-dihydropyrazolo[5,1-b]oxazol-6-yl)methyl)-6-((2-(1-methoxyethyl)thiazol-4-yl)sulfonyl)phthalazin-1(2H)-one;
2-((2,3-dihydropyrazolo[5,1-b]oxazol-6-yl)methyl)-6-((2-methylthiazol-4-yl)sulfonyl)phthalazin-1(2H)-one;
2-((5-methyl-1H-pyrazol-3-yl)methyl)-6-((2-methylthiazol-4-yl)sulfonyl)phthalazin-1(2H)-one;
2-((2,3-dihydrofuro[3,2-b]pyridin-5-yl)methyl)-6-((5-methyl-1H-pyrazol-3-yl)sulfonyl)phthalazin-1(2H)-one;
2-((2,3-dihydrofuro[3,2-b]pyridin-5-yl)methyl)-6-((1-(2-methoxyethyl)-1H-pyrazol-3-yl)sulfonyl)phthalazin-1(2H)-one;
2-((1H-pyrazol-3-yl)methyl)-6-((1-(2,2,2-trifluoroethyl)-1H-pyrazol-4-yl)sulfonyl)phthalazin-1(2H)-one;
2-[(7-methyl-2,3-dihydropyrazolo[5,1-b]oxazol-6-yl)methyl]-6-(1H-pyrazol-4-ylsulfonyl)phthalazin-1-one;
(R)-2-((2,3-dihydrofuro[3,2-b]pyridin-5-yl)methyl)-6-(1H-pyrazole-4-sulfonimidoyl)phthalazin-1(2H)-one; and
(S)-2-((2,3-dihydrofuro[3,2-b]pyridin-5-yl)methyl)-6-(1H-pyrazole-4-sulfonimidoyl)phthalazin-1(2H)-one;
or a pharmaceutically acceptable salt and/or solvate thereof.

In one embodiment is provided a compound selected from:
6-((4-methoxyphenyl)sulfonyl)phthalazin-1(2H)-one;
6-(phenylsulfonyl)phthalazin-1(2H)-one;
6-bromo-2-((6-methoxypyridin-3-yl)methyl)phthalazine-1(2H)-one;
6-mercapto-2-((6-methoxypyridin-3-yl)methyl)phthalazine-1(2H)-one;
tert-butyl (3-((6-bromo-1-oxophthalazin-2(1H)-yl)methyl)phenyl)carbamate;
tert-butyl (3-((6-mercapto-1-oxophthalazin-2(1H)-yl)methyl)phenyl)carbamate;
2-((2-chloropyridin-3-yl)methyl)-6-mercaptophthalazin-1(2H)-one;
6-mercapto-2-((1-methyl-1H-pyrazol-3-yl)methyl)phthalazine-1(2H)-one;
6-mercapto-2-((1-(tetrahydro-2H-pyran-2-yl)-1H-pyrazol-3-yl)methyl)phthalazine-1(2H)-one;
2-((1-cyclopropyl-1H-pyrazol-3-yl)methyl)-6-mercaptophthalazin-1(2H)-one;
6-bromo-2-((4-methyl-1-((2-(trimethylsilyl)ethoxy)methyl)-1H-pyrazol-3-yl)methyl)phthalazin-1(2H)-one;
6-bromo-2-((5-methyl-1H-pyrazol-3-yl)methyl)phthalazine-1(2H)-one;
6-mercaptophthalazin-1(2H)-one;
6-mercapto-2-((6-methylpyridin-2-yl)methyl)phthalazin-1(2H)-one;
5-fluoro-6-(phenylthio)phthalazin-1(2H)-one;
7-fluoro-6-(phenylthio)phthalazin-1(2H)-one; and
6-((1-(tetrahydro-2H-pyran-2-yl)-1H-pyrazol-4-yl)thio)phthalazin-1(2H)-one;
or a salt thereof.

Compounds of formula (Ia) may be synthesised as shown in the schemes below and as shown in the Example section.

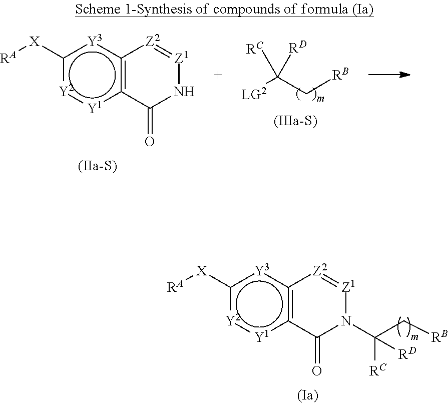

wherein R^{A}, R^{B}, R^{C}, R^{D}, Y¹, Y², Y³, Z¹, Z², X and m are defined elsewhere herein, and LG² is a leaving group such as halo (e.g. chloro, bromo or iodo), OMs or OTs. A compound of formula (Ila-S) may be reacted with a compound of formula (IIIa-S) in the presence of a base, such as Cs₂CO₃, K₂CO₃ or NaH, to provide a compound of formula (Ia).

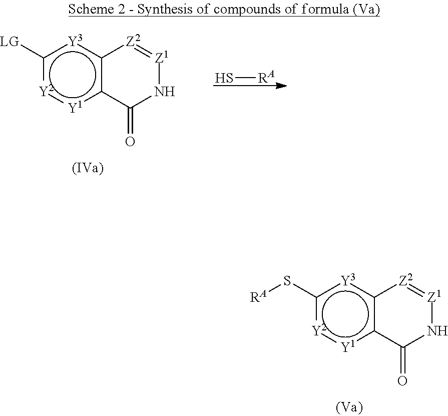

wherein R^{A}, Y¹, Y², Y³, Z¹ and Z² are defined elsewhere herein. In certain compounds of formula (Va), such compounds may be prepared by coupling a compound of formula (IVa) wherein LG is a leaving group such as halo, OMs or OTs, with HS—R^{A} wherein R^{A} is defined elsewhere herein. The S atom in compounds of formula (Va) may be converted to S═O or SO₂ under standard oxidation conditions such as oxone in DMF, 60° C. The oxidation may occur directly after the synthesis of compounds of formula (Va) or as a later step, for example, after coupling compounds of formula (Va) with compounds of formula (IIIa-S) as shown in Scheme 1.

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (Boc), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include alkyl esters (such as C₁₋₆ alkyl e.g. C₁₋₄ alkyl esters), benzyl esters and silyl esters.

In one embodiment, there is provided a process for preparing a compound of formula (Ia), or a salt, such as a pharmaceutically acceptable salt, thereof, which comprises reacting a compound of formula (IIa-S):

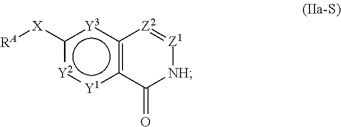

or a salt thereof;
with a compound of formula (IIIa-S):

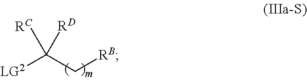

wherein R^{A}, R^{B}, R^{C}, R^{D}, Y¹, Y², Y³, Z¹, Z², X and m are defined elsewhere herein, and LG² is a leaving group such as halo (e.g. chloro, bromo or iodo), OMs or OTs.

In one embodiment, there is provided a compound of formula (IIa-S):

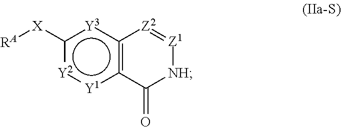

or a salt thereof;
wherein R^{A}, Y¹, Y², Y³, Z¹, Z² and X are defined elsewhere herein.

In one embodiment, there is provided a compound of formula (IVa):

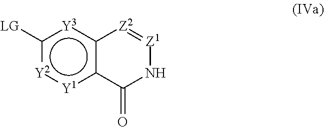

or a salt thereof;
wherein Y¹, Y², Y³, Z¹ and Z² are defined elsewhere herein and LG is a leaving group such as halo, OMs or OTs.

In one embodiment, there is provided a compound of formula (Va):

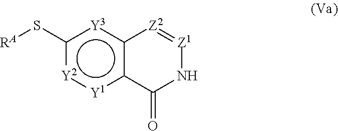

or a salt thereof;
wherein R^{A}, Y¹, Y², Y³, Z¹, and Z² are defined elsewhere herein.

In one embodiment, there is provided a compound of formula (VIa):

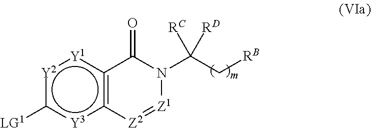

or a salt thereof;
wherein R^{B}, R^{C}, R^{D}, m, Y¹, Y², Y³, Z¹ and Z² are defined elsewhere herein and LG¹ is a leaving group such as halo (e.g. chloro, bromo or iodo), OMs or OTs.

In one embodiment, there is provided a compound of formula (VIIa):

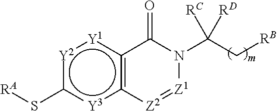

or a salt thereof;
wherein R^{A}, R^{B}, R^{C}, R^{D}, m, Y¹, Y², Y³, Z¹ and Z² are defined elsewhere herein.

It will be appreciated that for use in therapy the salts of the compounds of formula (Ia) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids, e.g., hydrochloric acid, hydrobromic acid, sulfuric acid, nitric acid or phosphoric acid. Also included are salts formed with organic acids, e.g., succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts, e.g., oxalates or formates, may be used, for example in the isolation of compounds of formula (Ia) and are included within the scope of this invention, as are basic addition salts such as sodium, potassium, calcium, aluminium, zinc, magnesium and other metal salts.

Pharmaceutically acceptable salts may also be formed with organic bases such as basic amines, e.g., with ammonia, meglumine, tromethamine, piperazine, arginine, choline, diethylamine, benzathine or lysine. Thus, in one embodiment there is provided a compound of formula (Ia) in the form of a pharmaceutically acceptable salt. Alternatively, there is provided a compound of formula (Ia) in the form of a free acid. When the compound contains a basic group as well as the free acid it may be zwitterionic.

Suitably, the compound of formula (Ia) is not a salt, e.g., is not a pharmaceutically acceptable salt.

Suitably, the compound of formula (Ia) or a pharmaceutically acceptable salt and/or solvate thereof is the pharmaceutically acceptable salt of the compound of formula (Ia).

Alternatively, the compound of formula (Ia) or a pharmaceutically acceptable salt and/or solvate thereof is the compound of formula (Ia).

Suitably, where the compound of formula (Ia) is in the form of a salt, the pharmaceutically acceptable salt is an acid addition salt such as an ammonium salt (e.g. formed with an inorganic acid such as HCl).

The compounds of formula (Ia) may be prepared in crystalline or non-crystalline form and, if crystalline, may optionally be solvated, e.g., as the hydrate. This invention includes within its scope stoichiometric solvates (e.g., hydrates) as well as compounds containing variable amounts of solvent (e.g., water). Suitably, the compound of formula (Ia) is not a solvate.

The invention extends to a pharmaceutically acceptable derivative thereof, such as a pharmaceutically acceptable prodrug of compounds of formula (Ia). Typical prodrugs of compounds of formula (Ia) which comprise a carboxylic acid include ester (e.g. C₁₋₆ alkyl e.g. C₁₋₄ alkyl ester) derivatives thereof. Thus, in one embodiment, the compound of formula (Ia) is provided as a pharmaceutically acceptable prodrug. In another embodiment, the compound of formula (Ia) is not provided as a pharmaceutically acceptable prodrug.

It is to be understood that the present invention encompasses all isomers of compounds of formula (Ia) including all geometric, tautomeric and optical forms, and mixtures thereof (e.g. racemic mixtures). In particular, the invention extends to all tautomeric forms of the compounds of formula (Ia). Where additional chiral centres are present in compounds of formula (Ia), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (Ia) are provided in a natural isotopic form. In one embodiment, the compounds of formula (Ia) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (Ia). In one embodiment, the atoms of the compounds of formula (Ia) are in an isotopic form which is not radioactive. In one embodiment, one or more atoms of the compounds of formula (Ia) are in an isotopic form which is radioactive. Suitably radioactive isotopes are stable isotopes. Suitably the unnatural variant isotopic form is a pharmaceutically acceptable form.

In one embodiment, a compound of formula (Ia) is provided whereby a single atom of the compound exists in an unnatural variant isotopic form. In another embodiment, a compound of formula (Ia) is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (Ia) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the purer forms used in the pharmaceutical compositions.

### Therapeutic Indications

Compounds of formula (Ia) are of use in therapy, particularly for treating or preventing an inflammatory disease, a disease associated with an undesirable immune response, cancer, obesity, a diabetic disease or a blood disorder. As shown in Biological Example 1 below, Example compounds of formula (Ia) tested exhibited improved modulatory activity for PKM2 compared with mitapivat. As shown in Biological Example 2 below, Example compounds of formula (Ia) tested exhibited improved modulatory activity for PKLR, again using mitapivat as comparator. As shown in Biological Example 3 below, Example compounds of formula (Ia) tested also exhibited an in vitro anti-proliferative effect. As such, compounds of formula (Ia) are expected to be suitable for the treatment of diseases associated with PK, in particular PKM2 and PKLR activity.

Thus, in a first aspect, the present invention provides a compound of formula (Ia) or a pharmaceutically acceptable salt and/or solvate thereof as defined herein, for use as a medicament. Also provided is a pharmaceutical composition comprising a compound of formula (Ia) or a pharmaceutically acceptable salt and/or solvate thereof as defined herein. Such a pharmaceutical composition contains the compound of formula (Ia) and one or more pharmaceutically acceptable diluents or carriers. Suitably, the invention provides the pharmaceutical composition as defined above for use as a medicament. The following fallbacks to the use of the compound of formula (Ia) apply equally to the pharmaceutical composition defined herein which comprises a compound of formula (Ia) or a pharmaceutically acceptable salt and/or solvate thereof as defined herein.

In a further aspect, the present invention provides a compound of formula (Ia) or a pharmaceutically acceptable salt and/or solvate as defined herein, for use in treating or preventing a disease, disorder or condition associated with the function of PK, in particular PKM2 and/or PKLR. In a further aspect, the present invention provides the use of a compound of formula (Ia) or a pharmaceutically acceptable salt and/or solvate thereof as defined herein, in the manufacture of a medicament for treating or preventing a disease, disorder or condition associated with the function of PK, in particular PKM2 and/or PKLR. In a further aspect, the present invention provides a method of treating or preventing a disease, disorder or condition associated with the function of PK, in particular PKM2 and/or PKLR, which comprises administering a compound of formula (Ia) or a pharmaceutically acceptable salt and/or solvate thereof as defined herein.

In a further aspect, the present invention provides a compound of formula (Ia) or a pharmaceutically acceptable salt and/or solvate as defined herein, for use in treating or preventing a symptom associated with a disease, disorder or condition associated with the function of PK, in particular PKM2 and/or PKLR. In a further aspect, the present invention provides the use of a compound of formula (Ia) or a pharmaceutically acceptable salt and/or solvate thereof as defined herein, in the manufacture of a medicament for treating or preventing a symptom associated with a disease, disorder or condition associated with the function of PK, in particular PKM2 and/or PKLR. In a further aspect, the present invention provides a method of treating or preventing a symptom associated with a disease, disorder or condition associated with the function of PK, in particular PKM2 and/or PKLR, which comprises administering a compound of formula (Ia) or a pharmaceutically acceptable salt and/or solvate thereof as defined herein.

In one embodiment, a compound of formula (Ia) is a modulator of PKM2. In another embodiment, a compound of formula (Ia) is an activator of PKM2. In one embodiment, a compound of formula (Ia) is a modulator of PKLR. In another embodiment, a compound of formula (Ia) is an activator of PKLR. A compound is an “activator” of PK (e.g. PKM2 and/or PKLR) if it increases the activity of the enzyme, which can be quantified by, for example, determining the concentration of ATP generated in a suitable assay (such as Biological Example 1 for PKM2 and Biological Example 2 for PKLR).

In a further aspect, the present invention provides a compound of formula (Ia) or a pharmaceutically acceptable salt and/or solvate thereof as defined herein, for use in treating or preventing an inflammatory disease, a disease associated with an undesirable immune response, cancer, obesity, a diabetic disease or a blood disorder. In a further aspect, the present invention provides the use of a compound of formula (Ia) or a pharmaceutically acceptable salt and/or solvate thereof as defined herein, in the manufacture of a medicament for treating or preventing an inflammatory disease, a disease associated with an undesirable immune response, cancer, obesity, a diabetic disease or a blood disorder. In a further aspect, the present invention provides a method of treating or preventing an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

In a further aspect, the present invention provides a compound of formula (Ia) or a pharmaceutically acceptable salt and/or solvate thereof as defined herein, for use in treating or preventing a symptom associated with an inflammatory disease, a disease associated with an undesirable immune response, cancer, obesity, a diabetic disease or a blood disorder. In a further aspect, the present invention provides the use of a compound of formula (Ia) or a pharmaceutically acceptable salt and/or solvate thereof as defined herein, in the manufacture of a medicament for treating or preventing a symptom associated with an inflammatory disease, a disease associated with an undesirable immune response, cancer, obesity, a diabetic disease or a blood disorder. In a further aspect, the present invention provides a method of treating or preventing a symptom associated with an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (Ia) or a pharmaceutically acceptable salt and/or solvate thereof as defined herein, for use in treating an inflammatory disease, a disease associated with an undesirable immune response, cancer, obesity, a diabetic disease or a blood disorder. In one embodiment of the invention is provided the use of a compound of formula (Ia) or a pharmaceutically acceptable salt and/or solvate thereof as defined herein, in the manufacture of a medicament for treating an inflammatory disease, a disease associated with an undesirable immune response, cancer, obesity, a diabetic disease or a blood disorder. In one embodiment of the invention is provided a method of treating an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating a symptom associated with an inflammatory disease, a disease associated with an undesirable immune response, cancer, obesity, a diabetic disease or a blood disorder. In one embodiment of the invention is provided the use of a compound of formula (Ia) or a pharmaceutically acceptable salt and/or solvate thereof as defined herein, in the manufacture of a medicament for treating a symptom associated with an inflammatory disease, a disease associated with an undesirable immune response, cancer, obesity, a diabetic disease or a blood disorder. In one embodiment of the invention is provided a method of treating a symptom associated with an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in preventing an inflammatory disease, a disease associated with an undesirable immune response, cancer, obesity, a diabetic disease or a blood disorder. In one embodiment of the invention is provided the use of a compound of formula (Ia) or a pharmaceutically acceptable salt and/or solvate thereof as defined herein, in the manufacture of a medicament for preventing an inflammatory disease, a disease associated with an undesirable immune response, cancer, obesity, a diabetic disease or a blood disorder. In one embodiment of the invention is provided a method of preventing an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in preventing a symptom associated with an inflammatory disease, a disease associated with an undesirable immune response, cancer, obesity, a diabetic disease or a blood disorder. In one embodiment of the invention is provided the use of a compound of formula (Ia) or a pharmaceutically acceptable salt and/or solvate thereof as defined herein, in the manufacture of a medicament for preventing a symptom associated with an inflammatory disease, a disease associated with an undesirable immune response, cancer, obesity, a diabetic disease or a blood disorder. In one embodiment of the invention is provided a method of preventing a symptom associated with an inflammatory disease, a disease associated with an undesirable immune response, cancer, obesity, a diabetic disease or a blood disorder, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing an inflammatory disease. In one embodiment of the invention is provided the use of a compound of formula (Ia) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with an inflammatory disease. In one embodiment of the invention is provided the use of a compound of formula (Ia) or a pharmaceutically acceptable salt and/or solvate thereof as defined herein, in the manufacture of a medicament for treating or preventing a symptom associated with an inflammatory disease. In one embodiment of the invention is provided a method of treating or preventing a symptom associated with an inflammatory disease, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing inflammation associated with an inflammatory disease. In one embodiment of the invention is provided the use of a compound of formula (Ia) or a pharmaceutically acceptable salt and/or solvate thereof as defined herein, in the manufacture of a medicament for treating or preventing inflammation associated with an inflammatory disease. In one embodiment of the invention is provided a method of treating or preventing inflammation associated with an inflammatory disease, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a disease associated with an undesirable immune response. In one embodiment of the invention is provided the use of a compound of formula (Ia) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with a disease associated with an undesirable immune response. In one embodiment of the invention is provided the use of a compound of formula (Ia) or a pharmaceutically acceptable salt and/or solvate thereof as defined herein, in the manufacture of a medicament for treating or preventing a symptom associated with a disease associated with an undesirable immune response. In one embodiment of the invention is provided a method of treating or preventing a symptom associated with a disease associated with an undesirable immune response, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing inflammation associated with a disease associated with an undesirable immune response. In one embodiment of the invention is provided the use of a compound of formula (Ia) or a pharmaceutically acceptable salt and/or solvate thereof as defined herein, in the manufacture of a medicament for treating or preventing inflammation associated with a disease associated with an undesirable immune response. In one embodiment of the invention is provided a method of treating or preventing inflammation associated with a disease associated with an undesirable immune response, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing cancer. In one embodiment of the invention is provided the use of a compound of formula (Ia) or a pharmaceutically acceptable salt and/or solvate thereof as defined herein, in the manufacture of a medicament for treating or preventing cancer. In one embodiment of the invention is provided a method of treating or preventing cancer, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with cancer. In one embodiment of the invention is provided the use of a compound of formula (Ia) or a pharmaceutically acceptable salt and/or solvate thereof as defined herein, in the manufacture of a medicament for treating or preventing a symptom associated with cancer. In one embodiment of the invention is provided a method of treating or preventing a symptom associated with cancer, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing obesity. In one embodiment of the invention is provided the use of a compound of formula (Ia) or a pharmaceutically acceptable salt and/or solvate thereof as defined herein, in the manufacture of a medicament for treating or preventing obesity. In one embodiment of the invention is provided a method of treating or preventing obesity, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with obesity. In one embodiment of the invention is provided the use of a compound of formula (Ia) or a pharmaceutically acceptable salt and/or solvate thereof as defined herein, in the manufacture of a medicament for treating or preventing a symptom associated with obesity. In one embodiment of the invention is provided a method of treating or preventing a symptom associated with obesity, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a diabetic disease. In one embodiment of the invention is provided the use of a compound of formula (Ia) or a pharmaceutically acceptable salt and/or solvate thereof as defined herein, in the manufacture of a medicament for treating or preventing a diabetic disease. In one embodiment of the invention is provided a method of treating or preventing a diabetic disease, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with a diabetic disease. In one embodiment of the invention is provided the use of a compound of formula (Ia) or a pharmaceutically acceptable salt and/or solvate thereof as defined herein, in the manufacture of a medicament for treating or preventing a symptom associated with a diabetic disease. In one embodiment of the invention is provided a method of treating or preventing a symptom associated with a diabetic disease, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a blood disorder. In one embodiment of the invention is provided the use of a compound of formula (Ia) or a pharmaceutically acceptable salt and/or solvate thereof as defined herein, in the manufacture of a medicament for treating or preventing a blood disorder. In one embodiment of the invention is provided a method of treating or preventing a blood disorder, which comprises administering a compound of formula (Ia) or a pharmaceutically acceptable salt and/or solvate thereof as defined herein.

In one embodiment is provided a compound of formula (Ia) or a pharmaceutically acceptable salt and/or solvate thereof as defined herein, for use in treating or preventing a symptom associated with a blood disorder. In one embodiment of the invention is provided the use of a compound of formula (Ia) or a pharmaceutically acceptable salt and/or solvate thereof as defined herein, in the manufacture of a medicament for treating or preventing a symptom associated with a blood disorder. In one embodiment of the invention is provided a method of treating or preventing a symptom associated with a blood disorder, which comprises administering a compound of formula (Ia) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, fibrosis, cystic fibrosis, pulmonary arterial hypertension (PAH, including idiopathic PAH), lung sarcoidosis, idiopathic pulmonary fibrosis, kidney fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune iymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, inflammatory bowel disease (including ulcerative colitis and Crohn's disease), atopic dermatitis, fibrosis, uveitis, cryopyrin-associated periodic syndromes, Muckle-Wells syndrome, juvenile idiopathic arthritis, chronic obstructive pulmonary disease and asthma.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

In one embodiment, the disease is rheumatoid arthritis.

In one embodiment, the disease is inflammatory bowel disease (including ulcerative colitis and Crohn's disease).

In one embodiment, the disease is atopic dermatitis.

In one embodiment, the disease is fibrosis.

In one embodiment, cancer is selected from the group consisting of acute lymphoblastic leukaemia, adult; acute lymphoblastic leukaemia, childhood; acute myeloid leukaemia, adult; adrenocortical carcinoma; adrenocortical carcinoma, childhood; aids-related lymphoma; aids-related malignancies; anal cancer; astrocytoma, childhood cerebellar; astrocytoma, childhood cerebral; Barrett's esophagus (pre-malignant syndrome); bile duct cancer, extrahepatic; bladder cancer; bladder cancer, childhood; bone cancer, osteosarcoma/malignant fibrous histiocytoma; brain stem glioma, childhood; brain tumour, adult; brain tumour, brain stem glioma, childhood; brain tumour, cerebellar astrocytoma, childhood; brain tumour, cerebral astrocytoma/malignant glioma, childhood; brain tumour, ependymoma, childhood; brain tumour, medulloblastoma, childhood; brain tumour, supratentorial primitive neuroectodermal tumours, childhood; brain tumour, visual pathway and hypothalamic glioma, childhood; brain tumour, childhood (other); breast cancer; breast cancer and pregnancy; breast cancer, childhood; breast cancer, male; bronchial adenomas/carcinoids, childhood; carcinoid tumour, childhood; carcinoid tumour, gastrointestinal; carcinoma, adrenocortical; carcinoma, islet cell; carcinoma of unknown primary; central nervous system lymphoma, primary; cerebellar astrocytoma, childhood; cerebral astrocytoma/malignant glioma, childhood; cervical cancer; childhood cancers; chronic lymphocytic leukaemia; chronic myelogenous leukaemia; chronic myeloproliferative disorders; clear cell sarcoma of tendon sheaths; colon cancer; colorectal cancer; colorectal cancer, childhood; cutaneous t-cell lymphoma; endometrial cancer; ependymoma, childhood; epithelial cancer, ovarian; oesophageal cancer; oesophageal cancer, childhood; Ewing's family of tumours; extracranial germ cell tumour, childhood; extragonadal germ cell tumour; extrahepatic bile duct cancer; eye cancer, intraocular melanoma; eye cancer, retinoblastoma; gallbladder cancer; gastric (stomach) cancer; gastric (stomach) cancer, childhood; gastrointestinal carcinoid tumour; germ cell tumour, extracranial, childhood; germ cell tumour, extragonadal; germ cell tumour, ovarian; gestational trophoblastic tumour; glioma, childhood brain stem; glioma, childhood visual pathway and hypothalamic; hairy cell leukaemia; head and neck cancer; hepatocellular (liver) cancer; hepatocellular (liver) cancer, adult (primary); hepatocellular (liver) cancer, childhood (primary); cancer of the esophagus; Hodgkin's lymphoma; Hodgkin's lymphoma, adult; Hodgkin's lymphoma, childhood; Hodgkin's lymphoma during pregnancy; hypopharyngeal cancer; hypothalamic and visual pathway glioma, childhood; intraocular melanoma; islet cell carcinoma (endocrine pancreas); cancer of the endocrine system (e.g., cancer of the thyroid, pancreas, parathyroid or adrenal glands); Kaposi's sarcoma; kidney cancer; laryngeal cancer; laryngeal cancer, childhood; leukaemia, acute lymphoblastic, adult; leukaemia, acute lymphoblastic, childhood; leukaemia, acute myeloid, adult; leukaemia, acute myeloid, childhood; leukaemia, chronic lymphocytic; leukaemia, chronic myelogenous; leukaemia, hairy cell; lymphocytic lymphoma; lip and oral cavity cancer; liver cancer, adult (primary); liver cancer, childhood (primary); lung cancer; lung cancer, non-small cell; lung cancer, small cell; lymphoblastic leukaemia, adult acute; lymphoblastic leukaemia, childhood acute; lymphocytic leukaemia, chronic; lymphoma, aids-related; lymphoma, central nervous system (primary); lymphoma, cutaneous t-cell; lymphoma, Hodgkin's, adult; lymphoma, Hodgkin's, childhood; lymphoma, Hodgkin's during pregnancy; lymphoma, non-Hodgkin's, adult; lymphoma, non-Hodgkin's, childhood; lymphoma, non-Hodgkin's during pregnancy; lymphoma, primary central nervous system; macroglobulinemia, Waldenstrom's; male breast cancer; malignant mesothelioma, adult; malignant mesothelioma, childhood; malignant thymoma; medulloblastoma, childhood; melanoma; melanoma, intraocular; Merkel cell carcinoma; mesothelioma, malignant; metastatic squamous neck cancer with occult primary; multiple endocrine neoplasia syndrome, childhood; multiple myeloma/plasma cell neoplasm; mycosis fungoides; myelodysplastic syndromes; myelogenous leukaemia, chronic; myeloid leukaemia, childhood acute; myeloma, multiple; myeloproliferative disorders, chronic; nasal cavity and paranasal sinus cancer; nasopharyngeal cancer; nasopharyngeal cancer, childhood; neoplastic cutaneous disease; neuroblastoma; non-Hodgkin's lymphoma, adult; non-Hodgkin's lymphoma, childhood; non-Hodgkin's lymphoma during pregnancy; non-small cell lung cancer; neoplasms of the central nervous system (e.g., primary CNS lymphoma, spinal axis tumors, medulloblastoma, brain stem gliomas or pituitary adenomas); oat-cell cancer; oral cancer, childhood; oral cavity and lip cancer; oropharyngeal cancer; osteosarcoma/malignant fibrous histiocytoma of bone; ovarian cancer; ovarian cancer, childhood; ovarian epithelial cancer; ovarian germ cell tumour; ovarian low malignant potential tumour; pediatric malignancy; pancreatic cancer; pancreatic cancer, childhood; pancreatic cancer, islet cell; paranasal sinus and nasal cavity cancer; parathyroid cancer; penile cancer; pheochromocytoma; pineal and supratentorial primitive neuroectodermal tumours, childhood; pituitary tumour; plasma cell neoplasm/multiple myeloma; pleuropulmonary blastoma; pregnancy and breast cancer; pregnancy and Hodgkin's lymphoma; pregnancy and non-Hodgkin's lymphoma; primary central nervous system lymphoma; primary liver cancer, adult; primary liver cancer, childhood; prostate cancer (particularly hormone-refractory); chronic or acute leukemia; solid tumors of childhood; hypereosinophilia; rectal cancer; renal cell (kidney) cancer; renal cell cancer, childhood; renal pelvis and ureter, transitional cell cancer; retinoblastoma; rhabdomyosarcoma, childhood; salivary gland cancer; salivary gland cancer, childhood; sarcoma, Ewing's family of tumours; sarcoma, Kaposi's; sarcoma (osteosarcoma)/malignant fibrous histiocytoma of bone; sarcoma, rhabdomyosarcoma, childhood; sarcomas of soft tissues; sarcoma, soft tissue, adult; sarcoma, soft tissue, childhood; Sezary syndrome; skin cancer; skin cancer, childhood; skin cancer (melanoma); skin carcinoma, Merkel cell; small cell lung cancer; dermatofibrosarcoma protuberans; small intestine cancer; soft tissue sarcoma, adult; soft tissue sarcoma, childhood; cancer of the head and neck; squamous neck cancer with occult primary, metastatic; stomach (gastric) cancer; stomach (gastric) cancer, childhood; supratentorial primitive neuroectodermal tumours, childhood; t-cell lymphoma, cutaneous; testicular cancer; thymoma, childhood; thymoma, malignant; thyroid cancer; thyroid cancer, childhood; transitional cell cancer of the renal pelvis and ureter; trophoblastic tumour, gestational; unknown primary site, cancer of, childhood; unusual cancers of childhood; ureter and renal pelvis, transitional cell cancer; urethral cancer; cancer of the ureter (e.g., renal cell carcinoma, carcinoma of the renal pelvis); cancer of the penis; gynecologic tumors; uterine cancer; uterine sarcoma; carcinoma of the fallopian tubes; carcinoma of the endometrium; vaginal cancer; carcinoma of the vagina; carcinoma of the vulva; visual pathway and hypothalamic glioma, childhood; vulvar cancer; Waldenstrom's macro globulinemia; and Wilms' tumour.

In one embodiment, cancer is selected from the group consisting of lung cancer; NSCLC (non-small cell lung cancer); oat-cell cancer; bone cancer; pancreatic cancer; skin cancer; dermatofibrosarcoma protuberans; cancer of the head and neck; cutaneous or intraocular melanoma; uterine cancer; ovarian cancer; colo-rectal cancer; anal cancer; stomach cancer; colon cancer; breast cancer; gynecologic tumors (e.g., uterine sarcomas, carcinoma of the fallopian tubes, carcinoma of the endometrium, carcinoma of the cervix, carcinoma of the vagina or carcinoma of the vulva); Hodgkin's Disease; hepatocellular cancer; cancer of the esophagus; small intestine cancer; cancer of the endocrine system (e.g., cancer of the thyroid, pancreas, parathyroid or adrenal glands); sarcomas of soft tissues; urethral cancer; cancer of the penis; prostate cancer (particularly hormone-refractory); chronic or acute leukemia; solid tumors of childhood; hypereosinophilia; lymphocytic lymphomas; bladder cancer; kidney cancer; cancer of the ureter (e.g., renal cell carcinoma, carcinoma of the renal pelvis); pediatric malignancy; neoplasms of the central nervous system (e.g., primary CNS lymphoma, spinal axis tumors, medulloblastoma, brain stem gliomas or pituitary adenomas); Barrett's esophagus (pre-malignant syndrome) and neoplastic cutaneous disease.

“Obesity” refers to a condition in which a subject has a body mass index of greater than or equal to 30. The body mass index (BMI) is according to the “NIH Clinical Guidelines on the Identification and Evaluation, and Treatment of Overweight and Obesity in Adults” (1998).

In one embodiment, administration of a compound of formula (Ia) to a subject reduces the BMI of the subject to less than 30, for example less than 29, less than 28, less than 27, less than 26, or less than 25. In one embodiment, a compound of formula (Ia) is used to treat or prevent aberrant or inappropriate weight gain, metabolic rate, or fat deposition, for example is used to treat anorexia, bulimia, obesity, diabetes, or hyperlipidemia (e.g., elevated triglycerides and/or elevated cholesterol), as well as disorders of fat or lipid metabolism. In one embodiment, a compound of formula (Ia) is used to treat or prevent metabolic syndrome.

In one embodiment, a compound of formula (Ia) is used to treat obesity associated with Prader-Willi Syndrome (PWS). In one embodiment, a compound of formula (Ia) is used to reduce body fat, prevent increased body fat, reduce cholesterol (e.g., total cholesterol and/or ratios of total cholesterol to HDL cholesterol), and/or reduce appetite in individuals having PWS associated obesity, and/or reduce comorbidities such as diabetes, cardiovascular disease, and stroke.

A “diabetic disease” refers to diabetes mellitus (“diabetes”) or a diabetic complication. The two main types of diabetes are (i) Type 1 diabetes resulting from the pancreas not producing insulin for which the usual treatment is insulin replacement therapy and (ii) Type 2 diabetes where patients either produce insufficient insulin or have insulin resistance. Diabetic complications include microvascular and macrovascular complications, and include coronary artery disease, peripheral artery disease, stroke, diabetic nephropathy, diabetic neuropathy, diabetic retinopathy, diabetic kidney disease and NASH.

In one embodiment, a “blood disorder” is selected from the group consisting of thalassemia (e.g. beta-thalassemia), hereditary spherocytosis, hereditary elliptocytosis, abetalipoproteinemia (or Bassen-Kornzweig syndrome), paroxysmal nocturnal hemoglobinuria, acquired hemolytic anaemia (e.g., congenital anaemias (e.g., enzymopathies)), and anaemia of chronic diseases.

### Administration

References to the compound of formula (Ia) in this section are taken to include the compound of formula (Ia) or a pharmaceutically acceptable salt and/or solvate thereof, suitably the compound of formula (Ia).

The compound of formula (Ia) is usually administered as a pharmaceutical composition. Thus, in one embodiment, is provided a pharmaceutical composition comprising a compound of formula (Ia) and one or more pharmaceutically acceptable diluents or carriers.

The compound of formula (Ia) may be administered by any convenient method, e.g. by oral, parenteral, buccal, sublingual, nasal, rectal, intrathecal or transdermal administration, and the pharmaceutical compositions adapted accordingly.

The compound of formula (Ia) may be administered topically to the target organ e.g. topically to the eye, lung, nose or skin. Hence the invention provides a pharmaceutical composition comprising a compound of formula (Ia) optionally in combination with one or more topically acceptable diluents or carriers.

A compound of formula (Ia) which is active when given orally can be formulated as a liquid or solid, e.g. as a syrup, suspension, emulsion, tablet, capsule or lozenge.

A liquid formulation will generally consist of a suspension or solution of the compound of formula (Ia) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (Ia) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (Ia) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen(R), specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (Ia) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (Ia), depending on the method of administration. The composition may contain from 0% to 99.9% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (Ia), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (Ia) is used in combination with a further therapeutic agent or agents. When the compound of formula (Ia) is used in combination with other therapeutic agents, the compounds may be administered either sequentially or simultaneously by any convenient route. Alternatively, the compounds may be administered separately.

When the compound of formula (Ia) is used for treating or preventing an inflammatory disease or a disease associated with an undesirable immune response, therapeutic agents which may be used in combination with the compound of formula (Ia) include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibitors (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BlyS, B lymphocyte stimulator) inhibitors (e.g. belimumab, blisibimod), other B cell targeted therapy including fusion proteins targeting both APRIL (A Proliferation-Inducing Ligand) and BlyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

When the compound of formula (Ia) is used for treating or preventing cancer, therapeutic agents which may be used in combination with the compound of formula (Ia) include active agents which are used in conjunction with cancer therapy, such as agents used as palliative treatments to ameliorate unwanted side effects. Therefore, in one embodiment, the additional therapeutic agent is an agent used as a palliative treatment such as selected from the group consisting of: antiemetic agents, medication intended to alleviate pain such as opioids, medication used to decrease high blood uric acid levels such as allopurinol or rasburicase, anti-depressants, sedatives, anti-convulsant drugs, laxatives, anti-diarrhoeal drugs and/or antacids.

In another embodiment, the additional therapeutic agent is an additional cancer treatment such as chemotherapy, a targeted therapy, immunotherapy and hormonal therapy.

Examples of chemotherapy agents include antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). In one embodiment, the additional therapeutic agent is a chemotherapy agent and is selected from the group consisting of Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Satraplatin, Streptozocin, Talaporfin, Tegafur-uracil, Temoporfin, Temozolomide, Teniposide, Tesetaxel, Testolactone, Tetranitrate, Thiotepa, Tiazofurin, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinfhmine, Vinorelbine, Vorinostat, and Zorubicin.

Examples of targeted therapies include tyrosine kinase inhibitors, cyclin-dependent kinase inhibitors, monoclonal antibodies and fusion proteins. In one embodiment, the additional therapeutic agent is selected from the group consisting of Axitinib, Bosutinib, Cediranib, dasatinib, erlotinib, imatinib, gefitinib, lapatinib, Lestaurtinib, Nilotinib, Semaxanib, Sorafenib, Sunitinib, Vandetanib, Alvocidib, Seliciclib, Herceptin, rituximab, Tositumomab, Cetuximab, Panitumumab, Trastuzumab, Alemtuzumab, Bevacizumab, Edrecolomab, Gemtuzumab, Aflibercept, Denileukin diftitox and Bexxar.

When the compound of formula (Ia) is used for treating or preventing obesity, therapeutic agents which may be used in combination with the compound of formula (Ia) include a gastric or pancreatic lipase inhibitor (such as orlistat); a lipid lowering agent (such as a statin, a fibrate, niacin or a derivative thereof (such as acipimox), lecithin, a bile acid sequesterant, ezetimibe, lomitapide, a phytosterol, an omega-3 supplement, a PCSK9 inhibitor); a CB-1 antagonist; a lipoxygenase inhibitor; a somostatin analogue; an insulin compound or insulin analogue (such as human insulin, insulin lispro, insulin aspart, insulin glulisine, insulin glargine, insulin degludec); an insulin sensitising agent such as a PPAR-gamma agonist, PPAR-alpha agonist or mixed PPAR-gamma/alpha agonist (such as metformin, pioglitazone or rosiglitazone); an insulin secretagogue (such as a nateglinide or repaglinide, or a sulfonylurea such as gliclazide, glimeperide, limepiride, glyburide); an SGLT2 inhibitor (such as dapagliflozin, canagliflozin or empagliflozin); an amylin analogue (such as pramlintide); a DPPIV inhibitor (such as sitagliptin, saxagliptin, linagliptin, alogliptin or vildagliptin); a GLP-1 agonist (such as albiglutide, dulaglutide, exenatide, liraglutide, semaglutide or lixisenatide); an alpha-glucosidase inhibitor (such as acarbose, miglitol or voglibose); a phosphodiesterase inhibitor (such as pentoxifylline); a glycogen phosphorylase inhibitor; an MCH-1 antagonist; a glucokinase activator; a glucagon antagonist; an insulin signalling agonist; a PTP1B inhibitor; a gluconeogenesis inhibitor; a GSK inhibitor or a galanin receptor agonist.

When the compound of formula (Ia) is used for treating or preventing a diabetic disease, therapeutic agents which may be used in combination with the compound of formula (Ia) include a gastric or pancreatic lipase inhibitor (such as orlistat); a lipid lowering agent (such as a statin, a fibrate, niacin or a derivative thereof (such as acipimox), lecithin, a bile acid sequesterant, ezetimibe, lomitapide, a phytosterol, an omega-3 supplement, a PCSK9 inhibitor); a CB-1 antagonist; a lipoxygenase inhibitor; a somostatin analogue; an insulin compound or insulin analogue (such as human insulin, insulin lispro, insulin aspart, insulin glulisine, insulin glargine, insulin degludec); an insulin sensitising agent such as a PPAR-gamma agonist, PPAR-alpha agonist or mixed PPAR-gamma/alpha agonist (such as metformin, pioglitazone or rosiglitazone); an insulin secretagogue (such as a nateglinide or repaglinide, or a sulfonylurea such as gliclazide, glimeperide, limepiride, glyburide); an SGLT2 inhibitor (such as dapagliflozin, canagliflozin or empagliflozin); an amylin analogue (such as pramlintide); a DPPIV inhibitor (such as sitagliptin, saxagliptin, linagliptin, alogliptin or vildagliptin); a GLP-1 agonist (such as albiglutide, dulaglutide, exenatide, liraglutide, semaglutide or lixisenatide); an alpha-glucosidase inhibitor (such as acarbose, miglitol or voglibose); a phosphodiesterase inhibitor (such as pentoxifylline); a glycogen phosphorylase inhibitor; an MCH-1 antagonist; a glucokinase activator; a glucagon antagonist; an insulin signalling agonist; a PTP1B inhibitor; a gluconeogenesis inhibitor; a GSK inhibitor or a galanin receptor agonist.

Compounds of formula (Ia) may display one or more of the following desirable properties:
low EC₅₀ and/or high Eₘₐₓ values for activating PKM2;
low EC₅₀ and/or high Eₘₐₓ values for activating PKLR;
low EC₅₀ and/or high Eₘₐₓ values for activating PKM2 and PKLR;
low IC₅₀ values for reducing cellular proliferation;
reduced dose and dosing frequency through improved pharmacokinetics;
improved oral systemic bioavailability;
reduced plasma clearance following intravenous dosing;
augmented cell permeability;
low toxicity at the relevant therapeutic dose.

Abbreviations

| | |
|---|---|
| Ac | acetyl |
| ADP | adenosine diphosphate |
| ADME | absorption, distribution, metabolism, and excretion |
| AIBN | azobisisobutyronitrile |
| Aq. | aqueous |
| ATP | adenosine triphosphate |
| BBFO | broadband fluorine observe |
| BEH | ethylene bridged hybrid |
| Bn | benzyl |
| Boc | tert-butyloxycarbonyl |
| CB-1 | cannabinoid-1 |
| CSH | charged surface hybrid |
| DAD | diode array detector |
| DBU | 1,8-diazabicyclo(5.4.0)undec-7-ene |
| DCE | dichloroethane |
| DCM | dichloromethane |
| DIPEA | N,N-diisopropylethylamine |
| DMF | dimethylformamide |
| DMSO | dimethyl sulfoxide |
| DPPIV | dipeptidyl peptidase-4 |
| ES⁺ | electrospray |
| Eq | equivalents |
| FBP | fructose-1,6-bisphosphate |
| FBS | fetal bovine serum |
| Fmoc | 9-fluorenylmethyloxycarbonyl |
| g | gram(s) |
| GLP-1 | glucagon-like peptide 1 |
| GSK | glycogen synthase kinase |
| h | hour(s) |
| HATU | (1-[bis(dimethylamino)methylene]-1H-1,2,3-triazolo[4,5- |
| | b]pyridinium 3-oxide hexafluorophosphate |
| HIF | hypoxia-inducible factor |
| HPLC | high-performance liquid chromatography |
| IL | interleukin |
| LCMS | liquid chromatography-mass spectrometry |
| M | molar concentration/molar mass |
| MCH | melanin-concentrating hormone |
| mCPBA | meta-chloroperoxybenzoic acid |
| mm | millimetre |
| (M)Hz | (mega)hertz |
| min(s) | minute(s) |
| mL | millilitres |
| mmol | millimole |
| MOM | methoxymethyl |
| MS | mass spectrometry |
| Ms | methanesulfonyl |
| MTBE | methyl tert-butyl ether |
| nm | nanometre |
| NASH | non-alcoholic fatty liver disease |
| NBS | N-bromosuccinimide |
| NCS | N-chlorosuccinimide |
| NMP | N-methyl-2-pyrrolidone |
| NMR | nuclear magnetic resonance |
| PBS | phosphate buffered saline |
| PDA | photodiode array |
| PEP | phosphoenolpyruvic acid |
| PK | pyruvate kinase |
| PMB | para-methoxybenzyl |
| PPAR | peroxisome proliferator-activated receptor |
| PTP1B | protein tyrosine phosphatase 1B |
| PTSA | para-toluenesulfonic acid |
| rpm | revolutions per minute |
| RT | room temperature |
| SEM | trimethylsilylethoxymethyl |
| SFC | supercritical fluid chromatography |
| SGLT2 | sodium-glucose transport protein 2 |
| STAT3 | signal transducer and activator of transcription 3 |
| TBAF | tetra-n-butylammonium fluoride |
| TBDMS | tert-butyldimethylsilyl |
| TCA | tricarboxylic acid cycle |
| TEA | triethylamine |
| Tf | trifluoromethanesulfonyl, i.e., CF₃SO₂— |
| TFA | trifluoroacetic acid |
| THF | tetrahydrofuran |
| THP | tetrahydropyranyl |
| TIPS | triisopropylsilyl |
| TMS | trimethylsilyl |
| TNF | tumour necrosis factor |
| TOM | tri-iso-propylsilyloxymethyl |
| T3P | propanephosphonic acid anhydride |
| Tr | trityl |
| TS | toluenesulfonyl |
| μL | microlitre |
| μM | micromolar |
| UPLC | ultra performance liquid chromatography |
| wt. | weight |
| ° C. | degrees centigrade |

### EXAMPLES

### Analytical Equipment

NMR spectra were recorded using a Bruker 400 MHz Avance III spectrometer fitted with a BBFO 5 mm probe, or a Bruker 500 MHz Avance III HD spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software.

UPLC/MS analysis was carried out on a Waters Acquity UPLC system using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH C18 column) or 10 mM Ammonium Bicarbonate (BEH C18 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

LCMS analysis was carried out on an Agilent LCMS system using either a Waters Acquity CSH C18 (4.6×30 mm) or BEH C18 column (4.6×30 mm) maintained at a temperature of 40° C. and eluted with a linear acetonitrile gradient appropriate for the lipophilicity of the compound over 4 or 15 minutes at a constant flow rate of 2.5 mL/min. The aqueous portion of the mobile phase was either 0.1% Formic Acid (CSH C18 column) or 10 mM Ammonium Bicarbonate (BEH C18 column). LC-UV chromatograms were recorded using an Agilent VWD or DAD detector at 254 nm. Mass spectra were recorded using an Agilent MSD detector with electrospray ionisation switching between positive and negative ion mode. Sample concentration was adjusted to give adequate UV response.

### Preparative HPLC Purification Methods

### Acidic Method (A):

Product was dissolved in DMSO (mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters Qda) using a Waters X-Select CSH C18 ODB prep column, 130 Å, 5 μm, 30 mm×100 mm, flow rate 40 mL min-1 eluting with a 0.1% formic acid in water-MeCN gradient over 12.5. At-column dilution pump gives 2 mL min-1 MeCN over the entire method, which is included in the following MeCN percentages. Gradient information: 0.0-0.5 min, lp % MeCN; 0.5-10.5 min, ramped from lp % MeCN to fp % MeCN; 10.5-10.6 min, ramped from fp % MeCN to 100% MeCN; 10.6-12.5 min, held at 100% MeCN. The clean fractions were evaporated in a Genevac.

### Basic Method (B):

Product was dissolved in DMSO (0.5 mL), filtered and purified by reversed phase preparative HPLC on a Waters X-Bridge BEH C18 ODB prep column, 130 Å, 5 μm, 30 mm×100 mm, flow rate 40 mL min-1 eluting with a 0.3% ammonia in water-MeCN gradient over 12.5 mins using UV detection across all wavelengths with PDA as well as a QDA and ELS detector. At-column dilution pump gives 2 mL min-1 MeCN over the entire method, which is included in the following MeCN percentages. Gradient information: 0.0-0.5 m A, 20% MeCN; 0.5-10.5 m, ramped from 20% MeCN to 50% MeCN; 10.5-10.6 min, ramped from 50% MeCN to 100% MeCN; 10.6-12.5 min, held at 100% MeCN. The clean fractions were evaporated in a Genevac.

Alternatively, the following analytical LCMS equipment and methods were also used:

LCMS/HPLC Instrument Details

| | | | ELS | Mass |
|---|---|---|---|---|
| System | Instrument Name | LC Detector | detector | detector |
| 1 | Agilent LCMS 1200 | G1315D DAD | 380 ELSD | Agilent |
| | | | | G6120B |
| 2 | Agilent LCMS 1200 | G1315C DAD | 380 ELSD | Agilent |
| | | | | G6110A |

LCMS/HPLC Method Details

| | | | | | | Column | Flow |
|---|---|---|---|---|---|---|---|
| Method | Solvent | | | UV | Mass | Temp. | Rate |
| Name | System | Column | Gradient | range | Range | ° C. | ml/min |
| A | A) water + | Waters | From 95:5 to | 190- | 100- | 40 | 2.0 |
| | 10 mM | X-Bridge | 0:100 in 1.6 | 400 | 1800 | | |
| | NH₄HCO₃ | C18 (50 | min, 0:100 for | nm | amu | | |
| | B) | mm × 4.6 | 1.4 min, from | | | | |
| | acetonitrile | mm × 3.5 | 0:100 to 95:5 | | | | |
| | | μm) | in 0.1 min, | | | | |
| | | | 95:5 for 0.7 | | | | |
| | | | min | | | | |
| B | A) water + | Waters | From 95:5 to | 190- | 100- | 40 | 2.0 |
| | 0.05% TFA | X-Bridge | 0:100 in 1.6 | 400 | 1100 | | |
| | B) | C18 (50 | min, 0:100 for | nm | amu | | |
| | acetonitrile + | mm × 4.6 | 1.4 min, from | | | | |
| | 0.05% TFA | mm × 3.5 | 0:100 to 95:5 | | | | |
| | | μm) | in 0.05 min, | | | | |
| | | | 95:5 for 0.7 | | | | |
| | | | min | | | | |
| C | A) water + | Halo C18 | From 95:5 to | 190- | 100- | 40 | 3.0 |
| | 0.05% TFA | (30 mm × | 0:100 in 0.8 | 400 | 1100 | | |
| | | 4.6 mm × | min, 0:100 for | nm | amu | | |
| | | 2.7 μm) | 0.4 min, from | | | | |
| | | | 0:100 to 95:5 | | | | |
| | | | in 0.01 min, | | | | |
| | | | 95:5 for 0.2 | | | | |
| | | | min | | | | |

### Commercial Materials

All starting materials and solvents were obtained either from commercial sources or prepared according to the literature citation.

### General Methods

Unless otherwise stated all reactions were stirred. Organic solutions were routinely dried over anhydrous magnesium sulfate. Hydrogenations were performed on a Thales H-cube flow reactor under the conditions stated or under pressure in a gas autoclave (bomb).

### General Route 1—Synthesis of Compounds of Formula (Ia) Wherein X is S(═O)

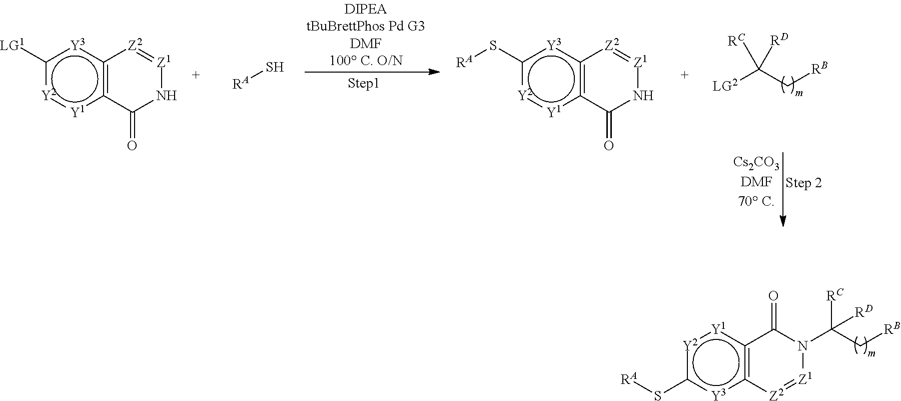

wherein R^{A}, R^{B}, R^{C}, R^{D}, Y¹, Y², Y³, Z¹, Z² and m are defined elsewhere herein and LG¹ and LG² are independently a leaving group such as halo (e.g. chloro, bromo or iodo), OMs or OTs.
General Route 1a—Synthesis of Compounds of Formula (Ia) Wherein X is S(═O)

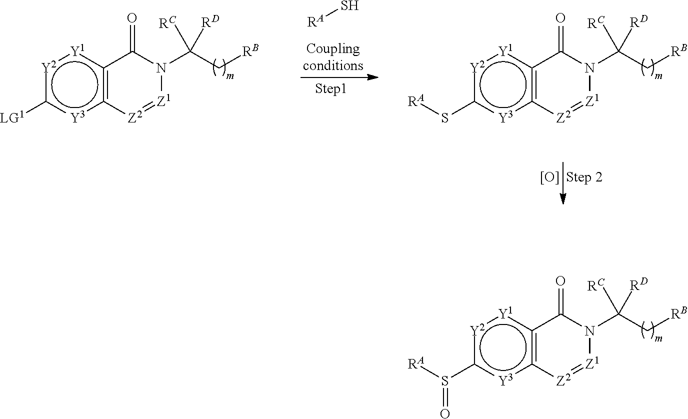

wherein R^{A}, R^{B}, R^{C}, R^{D}, Y¹, Y², Y³, Z¹, Z² and m are defined elsewhere herein and LG¹ is a leaving group such as halo (e.g. chloro, bromo or iodo), OMs or OTs. Coupling conditions are as described herein, for example in Example 119, and are known to the skilled person. The oxidation conditions in Step 2 may be as used in Example 119 i.e. NaIO₄, H₂O, DCM and MeOH at 40° C. Other suitable oxidation conditions are known to the skilled person.

### General Route 2—Synthesis of Compounds of Formula (Ia) Wherein X is S(═O)₂

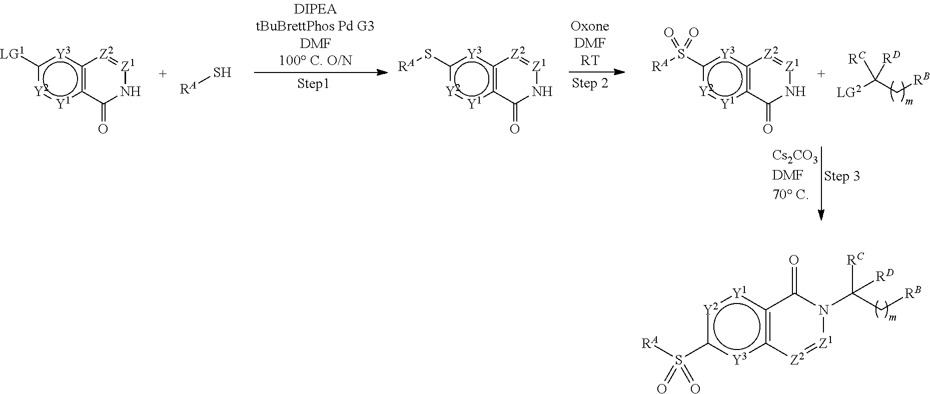

wherein R^{A}, R^{B}, R^{C}, R^{D}, Y¹, Y², Y³, Z¹, Z² and m are defined elsewhere herein, and LG¹ and LG² are independently a leaving group such as halo (e.g. chloro, bromo or iodo), OMs or OTs.

### General Route 3—Synthesis of Compounds of Formula (Ia) Wherein X is S(═O)₂

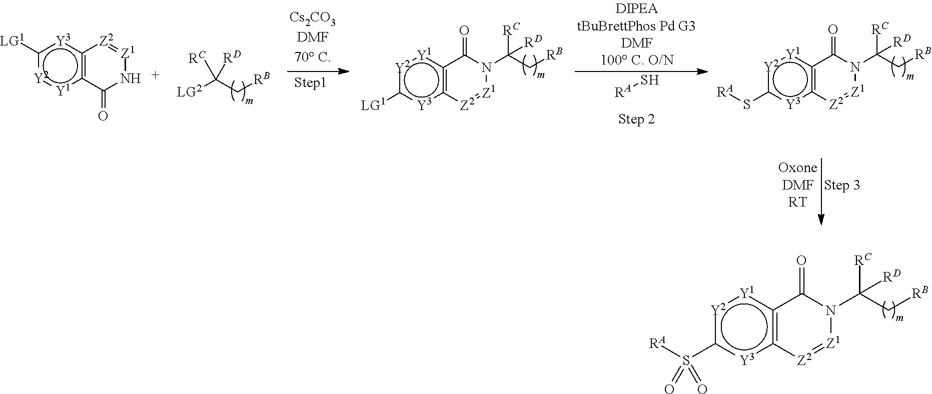

wherein R^{A}, R^{B}, R^{C}, R^{D}, Y¹, Y², Y³, Z¹, Z² and m are defined elsewhere herein, and LG¹ and LG² are independently a leaving group such as halo (e.g. chloro, bromo or iodo), OMs or OTs.

### General Route 4—Synthesis of Compounds of Formula (Ia) Wherein X is S(═O)₂

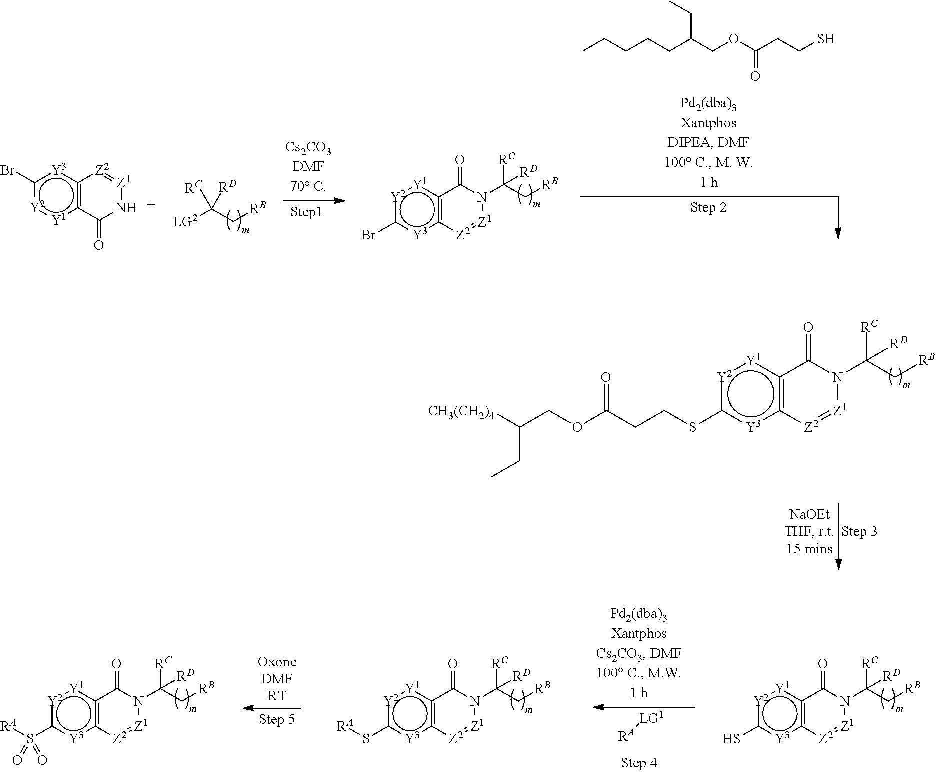

wherein R^{A}, R^{B}, R^{C}, R^{D}, Y¹, Y², Y³, Z¹, Z² and m are defined elsewhere herein, and LG¹ and LG² are independently a leaving group such as halo (e.g. chloro, bromo or iodo), OMs or OTs.

### General Route 5—Synthesis of Compounds of Formula (Ia) Wherein X is S(═O)₂

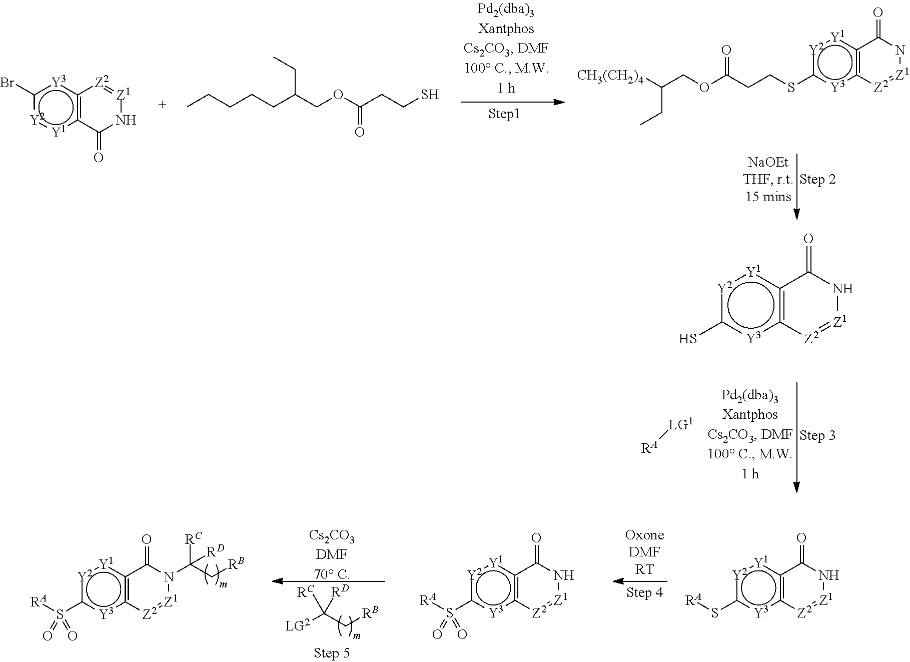

wherein R^{A}, R^{B}, R^{C}, R^{D}, Y¹, Y², Y³, Z¹, Z² and m are defined elsewhere herein, and LG¹ and LG² are independently a leaving group such as halo (e.g. chloro, bromo or iodo), OMs or OTs.

### General Procedure 1—Synthesis of Compounds Wherein R^{1B} is Hydroxyl

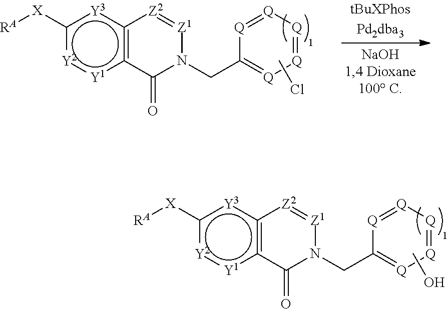

wherein R^{A}, Y¹, Y², Y³, Z¹ and Z² are defined elsewhere herein, and the moiety containing multiple Q groups represents phenyl, phenyl fused to a 5-7 membered heterocyclic ring, 5-10 membered heteroaryl or 5-6 membered heteroaryl fused to a 5-7 membered heterocyclic ring.

tBuXPhos (0.2 Eq) and Pd₂dba₃ (0.1 Eq) were added to a stirred solution of appropriate chloro-heterocyclic sulfone (1 Eq) in dry 1,4-dioxane (3 mL) under a nitrogen atmosphere. A 2N sodium hydroxide (10 Eq) solution was then added dropwise and the reaction mixture was stirred under nitrogen for 2 hours at 100° C. The reaction was cooled to room temperature and 1 N HCl (3 mL) was added, then extracted with DCM (2×5 mL). The organic phase was collected, dried (phase separator) and evaporated under reduced pressure. The crude product was purified by chromatography on silica gel to afford the desired hydroxyheterocyclic compound.

General Procedure 2—Synthesis of compounds wherein R^{1B} is an amine substituent

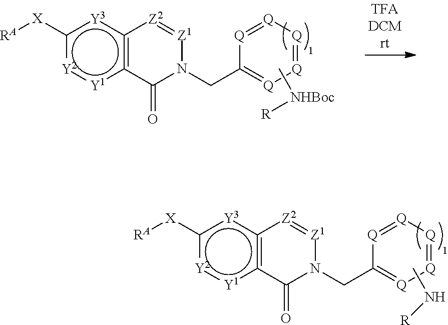

wherein R^{A}, Y¹, Y², Y³, Z¹ and Z² are defined elsewhere herein, and the moiety containing multiple Q groups represents phenyl, phenyl fused to a 5-7 membered heterocyclic ring, 5-10 membered heteroaryl or 5-6 membered heteroaryl fused to a 5-7 membered heterocyclic ring.

A stirred solution of the desired Boc protected amino heterocyclic compound (1 Eq, 1.18 mmol) in DCM (10 mL) was treated with TFA (1.48 g, 1.00 mL, 11.0 Eq, 13.0 mmol) dropwise. The reaction mixture was stirred at room temperature for 18 h. The reaction mixture was washed with 2 M NaOH (aq.) (10 mL) and the organic layer was collected. The aqueous was extracted with DCM (2×10 mL) and the combined organic extracts were dried (phase separator) and concentrated in vacuo to afford the crude product. The crude product was purified by chromatography on silica gel to afford the desired amino heterocyclic compound.

### General Procedure 3—THP Deprotection

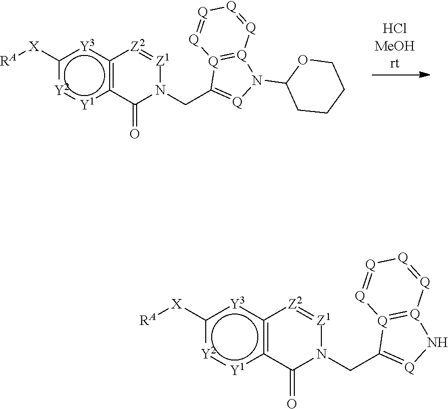

wherein R^{A}, Y¹, Y², Y³, Z¹ and Z² are defined elsewhere herein, and the moiety containing multiple Q groups represents 5-10 membered heteroaryl or 5-6 membered heteroaryl fused to a 5-7 membered heterocyclic ring, wherein the moiety contains NH.

HCl (4N in dioxane) (4 molar, 15 Eq) was added to a solution of the appropriate THP-protected N-heterocycle (1 Eq) in MeOH (0.5 mL) or alternatively in DCM (0.5 mL) and the mixture was stirred for 1 h, then concentrated under vacuum. Sat. NaHCO₃ and DCM were added and the layers separated through a phase separator. The organic layer was washed with brine, then absorbed on silica. The crude product was purified by chromatography on silica gel to afford the desired NH-heterocycle.

### Intermediate 1—6-((4-methoxyphenyl)sulfonyl)phthalazin-1(2H)-one

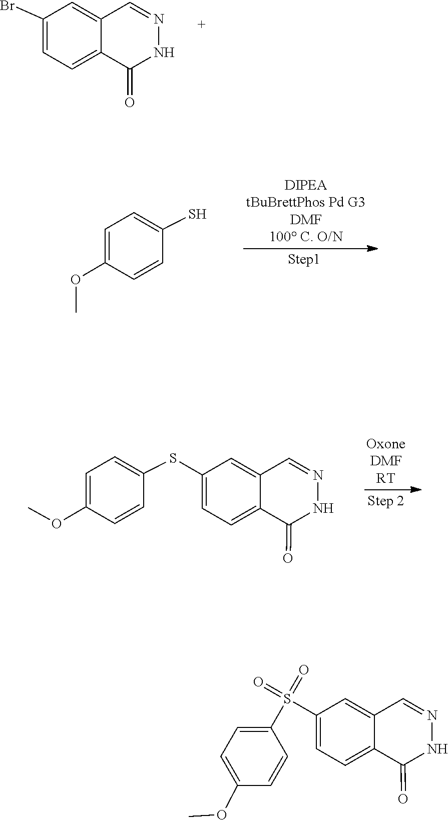

### Step 1

Nitrogen was bubbled through a mixture of 6-bromophthalazin-1(2H)-one (1.6 g, 1 Eq, 7.1 mmol), 4-methoxybenzenethiol (1.0 g, 0.87 mL, 1 Eq, 7.1 mmol) and DIPEA (1.8 g, 2.5 mL, 2 Eq, 14 mmol) in DMF (12 mL) and the mixture was stirred for 5 minutes. tBuBrettPhos Pd G3 (0.30 g, 0.05 Eq, 0.36 mmol) was added and the mixture was stirred at 100° C. under nitrogen overnight, then allowed to cool to room temperature. The mixture was poured into ice and the resulting precipitate washed with water, then DCM and MTBE to give crude 6-((4-methoxyphenyl)thio)phthalazin-1(2H)-one (1.632 g, 91% Purity) as an orange solid. MS(ES⁺): 285 (M+H)⁺

### Step 2

OXONE® (10.59 g, 3 Eq, 17.22 mmol) was added to a solution of 6-((4-methoxyphenyl)thio)phthalazin-1(2H)-one (1.632 g, 1 Eq, 5.740 mmol) in DMF (55 mL) and the reaction mixture was left to stir for 3 hours at RT. The reaction was diluted with water (100 mL) and the resulting precipitate was filtered, washed with water then MTBE, giving the title compound (1.031 g) as an off-white solid. MS(ES⁺): 317 (M+H)⁺

### Intermediate 2—6-(phenylsulfonyl)phthalazin-1(2H)-one

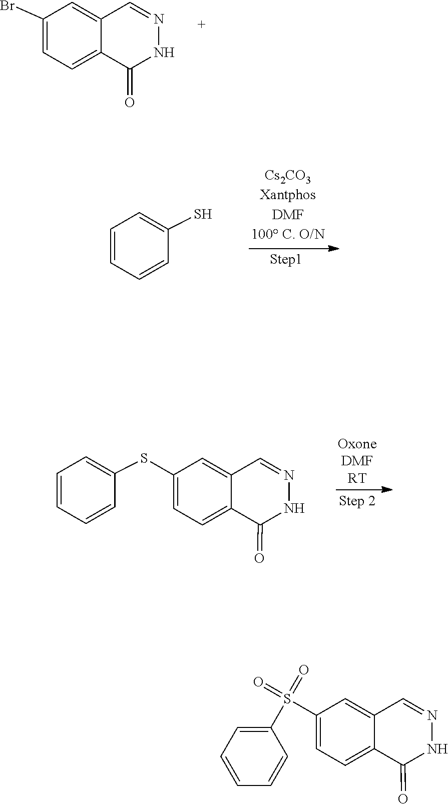 [SMILES: O=c1[nH]ncc2cc(Br)ccc12 ; Sc1ccccc1 ; O=c1[nH]ncc2cc(Sc3ccccc3)ccc12 ; O=c1[nH]ncc2cc(S(=O)(=O)c3ccccc3)ccc12]

### Step 1

A suspension of 6-bromophthalazin-1(2H)-one (2.00 g, 98% Wt, 1 Eq, 8.71 mmol) and cesium carbonate (4.54 g, 1.6 Eq, 13.9 mmol) in DMF (20 mL) was sparged with N₂ for 5 min. Pd₂(dba)₃ (399 mg, 0.05 Eq, 435 μmol), xantphos (504 mg, 0.1 Eq, 871 μmol) and thiophenol (1.44 g, 1.35 mL, 1.50 Eq, 13.1 mmol) were added sequentially. The reaction mixture was stirred at 100° C. under N₂ for 18 h, allowed to cool to RT and then poured into water (100 mL). The precipitate was collected by filtration, washing with water, and dried in vacuo to afford 6-(phenylthio)phthalazin-1(2H)-one (3.084 g, 90% Purity) as a red solid. The product was used without further purification in the next step. MS(ES⁺): 255 (M+H)⁺

### Step 2

A stirred suspension of 6-(phenylthio)phthalazin-1(2H)-one (3.084 g, 90% Wt, 1 Eq, 10.91 mmol) in DMF (60 mL) was treated with OXONE® (15.677 g, 2.337 Eq, 25.502 mmol) in several portions. The reaction mixture was stirred at RT for 18 h. The reaction mixture was concentrated in vacuo and the residue was treated with water (150 mL). The precipitate was collected by filtration, washing with water, and then dried in vacuo to afford 6-(phenylsulfonyl)phthalazin-1(2H)-one as a pale yellow solid. The crude product was purified by chromatography on silica gel to afford the title compound (2.448 g). MS(ES⁺): 287 (M+H)⁺

### Intermediate 3—5-(chloromethyl)-2-methoxypyridine hydrochloride

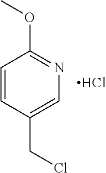

A stirred solution of (6-methoxypyridin-3-yl)methanol (2.00 g, 1.73 mL, 1 Eq, 14.4 mmol) in toluene (10 mL) was treated with thionyl chloride (1.88 g, 1.15 mL, 1.1 Eq, 15.8 mmol) dropwise. The resulting suspension was stirred at room temperature for 18 h. The reaction mixture was concentrated in vacuo to afford the title compound (2.806 g, 13 mmol, 91%) as a sticky yellow gum. ¹H NMR (400 MHz, DMSO-d6) δ 8.23 (d, J=2.5 Hz, 1H), 7.78 (dd, J=8.6, 2.5 Hz, 1H), 6.85 (d, J=8.5 Hz, 1H), 4.76 (s, 2H), 3.85 (s, 3H). 1 proton not observed in DMSO.

### Intermediate 4—tert-butyl 4-(chloromethyl)indoline-1-carboxylate

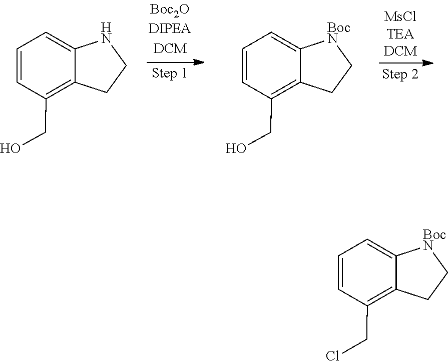 [SMILES: OCc1cccc2c1CCN2 ; CC(C)(C)OC(=O)N1CCc2c(CO)cccc21 ; CC(C)(C)OC(=O)N1CCc2c(CCl)cccc21]

### Step 1

Boc-anhydride (402 mg, 423 μL, 1.1 Eq, 1.84 mmol) was added to a stirred solution of indolin-4-ylmethanol (250 mg, 1 Eq, 1.68 mmol) and DIPEA (238 mg, 321 μL, 1.1 Eq, 1.84 mmol) in dry DCM (8 mL). The reaction mixture was stirred for 18 hours at RT. 1N HCl (10 mL) and DCM (5 mL) were added and the layers separated through a phase separating cartridge. The organic layer was washed with water (10 mL), brine (10 mL), dried (MgSO₄) and concentrated under vacuo to afford crude tert-butyl 4-(hydroxymethyl)indoline-1-carboxylate (0.41 g, 1.6 mmol, 93%) as an off-white solid. MS (ES⁺): 272 (M+Na)⁺.

### Step 2

A mixture of tert-butyl 4-(hydroxymethyl)indoline-1-carboxylate (100 mg, 95% Wt, 1 Eq, 381 μmol), triethylamine (38.6 mg, 53.1 μL, 1 Eq, 381 μmol), and mesyl-Cl (43.6 mg, 29.7 μL, 1 Eq, 381 μmol) in DCM (6.0 mL) was stirred at RT over three days. The reaction mixture was evaporated under reduced pressure and purified by chromatography on silica gel to afford the title compound (0.11 g) as a clear colourless oil. MS (ES⁺): 212/214 (M−tBu+H)⁺.

### Intermediate 5—3-(chloromethyl)-1-(tetrahydro-2H-pyran-2-yl)-1H-pyrazole

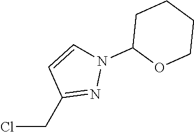 [SMILES: ClCc1ccn(C2CCCCO2)n1]

A suspension of 3-(chloromethyl)-1H-pyrazole, HCl (500 mg, 96% Wt, 1 Eq, 3.14 mmol) in DCM was treated with 3,4-dihydro-2H-pyran (286 mg, 310 μL, 97% Wt, 1.05 Eq, 3.29 mmol) dropwise. The reaction mixture was stirred at RT for 24 h. DCM (10 mL) and sat. aq. NaHCO₃ (10 mL) were added and the phases were mixed. The mixture was passed through a phase separator and the aqueous was extracted with DCM (2×10 mL). The combined organic extracts were dried (phase separator) and concentrated in vacuo to afford the title compound (501 mg) as a pale yellow oil. The product was used without further purification in the next step. MS (ES⁺): 435 223 (M+Na)⁺.

### Intermediate 6—3-(chloromethyl)-1-cyclopropyl-1H-pyrazole hydrochloride

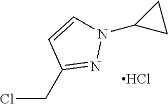

SOCl₂ (206 μL, 2.82 mmol, 3.00 equiv.) was added dropwise to a stirred solution of (1-cyclopropyl-1H-pyrazol-3-yl)methanol (130 mg, 941 μmol, 1 equiv.) in DCM (5 mL) at 0° C. The resulting mixture was stirred for 20 h at room temperature, then evaporated under reduced pressure. The residue was co-evaporated with toluene for 3 times, giving the title compound (0.16 g) as a dark orange oil. MS (ES⁺): 157/159 (M+H)⁺.

### Intermediate 7—2-chloro-3-(chloromethyl)-6-methoxypyridine

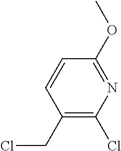

DIPEA (0.11 g, 0.15 mL, 1.5 Eq, 0.86 mmol) was added to a stirred solution of (2-chloro-6-methoxypyridin-3-yl)methanol (0.10 g, 1 Eq, 0.58 mmol) and methanesulfonyl chloride (73 mg, 49 μL, 1.1 Eq, 0.63 mmol) in dry DCM (6 mL) under a nitrogen atmosphere at 0° C. The reaction mixture was stirred for 2 hours at 0° C., then to warm to RT and stirred for 20 h. The reaction mixture was evaporated under reduced pressure and crude was used without further purification in the next step. MS (ES⁺): 192/194 (M+H)⁺.

### Intermediate 8—ethyl 3-(chloromethyl)-1-(tetrahydro-2H-pyran-2-yl)-1H-pyrazole-4-carboxyl

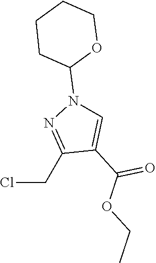 [SMILES: CCOC(=O)c1cn(C2CCCCO2)nc1CCl]

A stirred solution of ethyl 3-(chloromethyl)-1H-pyrazole-4-carboxylate (250 mg, 1 Eq, 1.33 mmol) and 4-methylbenzenesulfonic acid hydrate (30 mg, 0.12 Eq, 0.16 mmol) in DCM (5 mL) was treated with 3,4-dihydro-2H-pyran (138 mg, 150 μL, 97% Wt, 1.20 Eq, 1.59 mmol). The reaction mixture was stirred at RT for 22 h. The reaction mixture was washed with sat. aq. NaHCO₃ (10 mL). The organic layer was collected and the aqueous was extracted with DCM (2×5 mL). The combined organic extracts were dried (phase separator) and concentrated in vacuo to afford the title compound (356 mg) as an orange oil. ¹H NMR (400 MHz, DMSO-d6) δ 8.45 (s, 1H), 5.44 (dd, J=10.0, 2.4 Hz, 1H), 4.84 (s, 2H), 4.24 (q, J=7.1 Hz, 2H), 3.93 (d, J=11.8 Hz, 1H), 3.68-3.57 (m, 1H), 2.16-2.02 (m, 1H), 1.91 (dq, J=7.1, 3.9 Hz, 2H), 1.71-1.60 (m, 1H), 1.53 (tq, J=8.0, 3.8 Hz, 2H), 1.28 (t, J=7.1 Hz, 3H).

### Intermediate 9—3-(chloromethyl)-1,4-dimethyl-1H-pyrazole hydrochloride

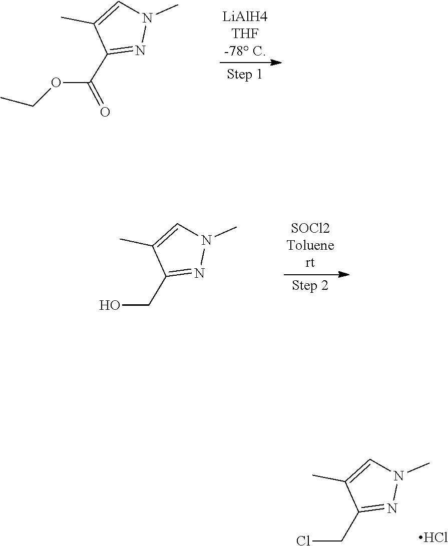

### Step 1

LiAlH₄ (56.4 mg, 372 μL, 4.0 molar, 1.0 Eq, 1.49 mmol) was added dropwise to a stirred solution of ethyl 1,4-dimethyl-1H-pyrazole-3-carboxylate (0.250 g, 1 Eq, 1.49 mmol) in THF (4.0 mL) at −78° C. and the resulting mixture was stirred at that temperature for 1 h before being warmed to 0° C., and stirring continued for a further 1 h. The reaction was then cautiously quenched by addition of saturated aqueous Rochelle salt (15 mL). The solution was allowed to warm to RT and was left stirring rigorously until the two phases were transparent. The phases were separated and the aqueous phase was extracted with EtOAC (3×30 mL). The combined organic phases were dried (MgSO₄), then concentrated under reduced pressure. The crude product was purified by chromatography on silica gel to afford (1,4-dimethyl-1H-pyrazol-3-yl)methanol (0.160 g) as a clear oil. MS (ES⁺): 127 (M+H)⁺.

### Step 2

Thionyl chloride (166 mg, 102 μL, 1.1 Eq, 1.40 mmol) was added dropwise to a stirred solution of (1,4-dimethyl-1H-pyrazol-3-yl)methanol (0.160 g, 1 Eq, 1.27 mmol) in toluene (3.5 mL) and the resulting suspension was stirred at RT for 18 h. The reaction mixture was concentrated in vacuo to afford the title compound (0.205 g) as a white solid. ¹H NMR (400 MHz, DMSO-d6) δ 10.72 (s, 1H), 7.43 (s, 1H), 4.65 (s, 2H), 3.74 (s, 3H), 2.01 (d, J=0.8 Hz, 3H).

### Intermediate 10—3-(chloromethyl)-4-methyl-1-((2-(trimethylsilyl)ethoxy)methyl)-1H-pyrazole

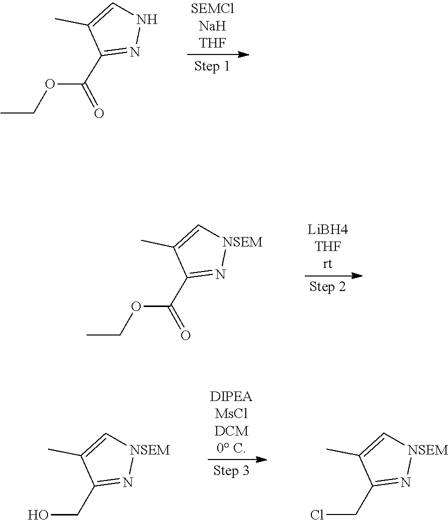 [SMILES: CCOC(=O)c1n[nH]cc1C ; CCOC(=O)c1nn(COCC[Si](C)(C)C)cc1C ; Cc1cn(COCC[Si](C)(C)C)nc1CO ; Cc1cn(COCC[Si](C)(C)C)nc1CCl]

### Step 1

SEM-Cl (0.39 g, 0.41 mL, 1.2 Eq, 2.3 mmol) was added dropwise to a suspension of ethyl 4-methyl-1H-pyrazole-3-carboxylate (0.30 g, 1 Eq, 1.9 mmol) and NaH (93 mg, 60% Wt, 1.2 Eq, 2.3 mmol) in THF (20 mL) at 0° C. The reaction mixture was allowed to warm to RT and stirred for 1 h. The reaction mixture was quenched with saturated aqueous ammonium chloride (30 mL) and extracted with DCM (2×20 mL). The organic phase was collected, dried (phase separator) and evaporated under reduced pressure. The crude product was purified by chromatography on silica gel to afford ethyl 4-methyl-1-((2-(trimethylsilyl)ethoxy)methyl)-1H-pyrazole-3-carboxylate (0.42 g) as a pale yellow oil. MS (ES⁺): 285 (M+H)⁺.

### Step 2

Lithium borohydride (61 mg, 1.4 mL, 2.00 molar, 2 Eq, 2.8 mmol) was added dropwise to a stirred solution of ethyl 4-methyl-1-((2-(trimethylsilyl)ethoxy)methyl)-1H-pyrazole-3-carboxylate (0.40 g, 1 Eq, 1.4 mmol) in dry THF (10.0 mL). The reaction mixture was stirred for 24 hours at RT. The reaction mixture was diluted with water (25 mL) and transferred into a separating funnel. The layer was extracted with EtOAc (2×25 mL). The combined organic layers was collected, dried (MgSO₄), filtered and concentrated in vacuo. The crude product was purified by chromatography on silica gel to afford (4-methyl-1-((2-(trimethylsilyl)ethoxy)methyl)-1H-pyrazol-3-yl)methanol (0.35 g) as a clear yellow solid. MS (ES⁺): 243 (M+H)⁺.

### Step 3

DIPEA (0.28 g, 0.38 mL, 1.5 Eq, 2.2 mmol) was added to a stirred solution of (4-methyl-1-((2-(trimethylsilyl)ethoxy)methyl)-1H-pyrazol-3-yl)methanol (0.35 g, 1 Eq, 1.4 mmol) and methanesulfonyl chloride (0.18 g, 0.12 mL, 1.1 Eq, 1.6 mmol) in dry DCM (10 mL) under a nitrogen atmosphere at 0° C. The reaction mixture was stirred for 2 hours at 0° C., then allowed to warm to RT and stirred overnight. The reaction mixture was evaporated under reduced pressure to afford the title compound (0.35 g, 85% purity). MS (ES⁺): 261/263 (M+H)⁺.

### Intermediate 11—methyl 2-bromo-2-(6-methoxypyridin-3-yl)acetate

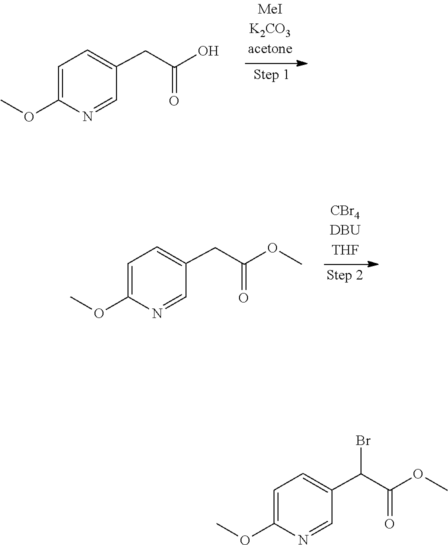

### Step 1

To a stirred solution of 2-(6-methoxypyridin-3-yl)acetic acid (1.000 g, 1 Eq, 5.982 mmol) in acetone (25 mL) at RT was added potassium carbonate (1.240 g, 1.5 Eq, 8.973 mmol) followed by methyl iodide (2.547 g, 1.122 mL, 3.0 Eq, 17.95 mmol). The resulting mixture was heated to 60° C. and stirring continued overnight. The reaction mixture was concentrated under reduced pressure, diluted in DCM (10 mL), and washed with water (2×10 mL). The organic layer was dried over MgSO₄ and concentrated under reduced pressure to afford a crude product. The crude product was purified by chromatography on silica gel to afford methyl 2-(6-methoxypyridin-3-yl)acetate (0.957 g) as a translucent oil ¹H NMR (400 MHz, DMSO-d6) δ 8.04 (d, J=2.5 Hz, 1H), 7.60 (dd, J=8.5, 2.5 Hz, 1H), 6.78 (dd, J=8.5, 0.8 Hz, 1H), 3.82 (s, 3H), 3.64 (s, 2H), 3.62 (s, 3H). MS (ES⁺): 182.1 (M+H)⁺.

### Step 2

DBU (458 μL, 1.1 Eq, 3.04 mmol) was added to a stirred solution of methyl 2-(6-methoxypyridin-3-yl)acetate (0.500 g, 1 Eq, 2.76 mmol) in dry THF (60 mL) at 0° C. The reaction mixture was allowed to warm to RT over 30 minutes, then cooled to −78° C. CBr₄ (1.01 g, 1.1 Eq, 3.04 mmol) was added and stirring was continued at −78° C. for 2 hours, then 2 hours at 0° C. The reaction mixture was quenched with sat. aq. NH₄Cl (10 mL) and the phases were separated. The organic phase was washed (brine), dried (Na₂SO₄), filtered, and concentrated in vacuo. The crude product was purified by chromatography on silica gel (12 g cartridge, 0-50% EtOAc/DCM) to afford the title compound (0.188 g) as a translucent oil. MS (ES⁺): 260/262 (M+H)⁺.

### Intermediate 12—6-(bromomethyl)-1-(tetrahydro-2H-pyran-2-yl)-1H-indazole

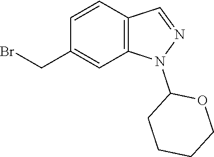 [SMILES: BrCc1ccc2cnn(C3CCCCO3)c2c1]

A yellow suspension of 6-(bromomethyl)-1H-indazole hydrogen bromide (0.12 g, 0.4 mmol) and 3,4-dihydro-2H-pyran (0.1 mL, 1.0 mmol) in THF (10.0 mL) was heated at reflux for 2 h. After cooling to RT, the reaction mixture was stirred for 12 h under N₂. The reaction mixture was diluted with methylene chloride (25 mL), washed with sat. aq. NaHCO₃ (10 mL), water (10 mL) and brine (10 mL). After drying (MgSO₄), the solvent was removed under reduced pressure, giving the title compound (0.15 g) as a brown oil. MS (ES)⁺: 295/297 (M+H)⁺.

### Intermediate 13—3-(chloromethyl)-4-fluoro-1-(tetrahydro-2H-pyran-2-yl)-1H-pyrazole

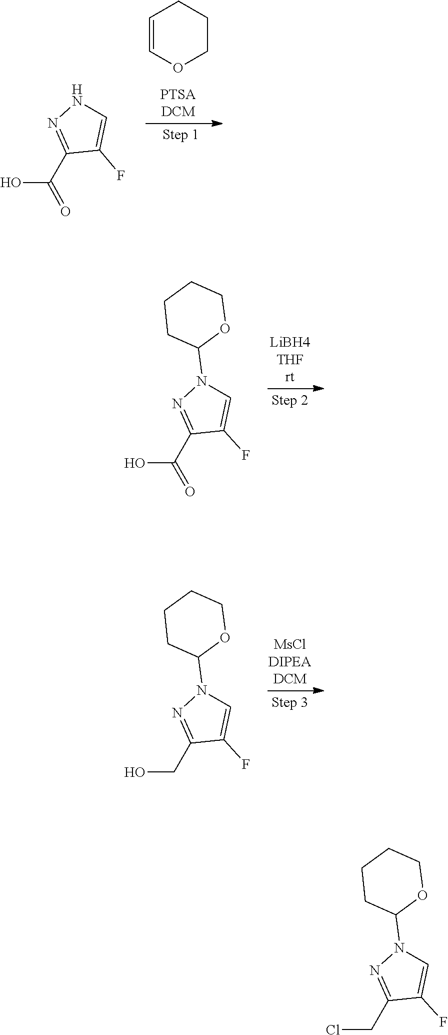 [SMILES: C1=COCCC1 ; O=C(O)c1n[nH]cc1F ; O=C(O)c1nn(C2CCCCO2)cc1F ; OCc1nn(C2CCCCO2)cc1F ; Fc1cn(C2CCCCO2)nc1CCl]

### Step 1

A stirred solution of methyl 4-fluoro-1H-pyrazole-3-carboxylate (120 mg, 1 Eq, 833 μmol) and p-toluenesulfonic acid monohydrate (79.2 mg, 0.5 Eq, 416 μmol) in DCM (12 mL) was treated with 3,4-dihydro-2H-pyran (108 mg, 117 μL, 97% Wt, 1.50 Eq, 1.25 mmol). The reaction mixture was stirred at RT for 2 h. The reaction mixture was washed with sat. aq. NaHCO₃ (10 mL). The organic layer was collected and the aqueous was extracted with DCM (2×5 mL). The combined organic extracts were dried (phase separator) and concentrated in vacuo to afford 4-fluoro-1-(tetrahydro-2H-pyran-2-yl)-1H-pyrazole-3-carboxylate (0.20 g) as an orange oil. ¹H NMR (400 MHz, DMSO-d6) δ 8.23 (dd, J=4.9, 2.2 Hz, 1H), 5.41 (dd, J=9.6, 2.4 Hz, 1H), 3.96-3.77 (m, 4H), 3.77-3.58 (m, 1H), 2.18-1.96 (m, 1H), 1.96-1.84 (m, 2H), 1.80-1.59 (m, 1H), 1.60-1.26 (m, 2H). MS (ES)⁺: 251 (M+Na)⁺.

### Step 2

Lithium borohydride (38.2 mg, 876 μL, 2.00 molar, 2 Eq, 1.75 mmol) was added dropwise to a stirred solution of methyl 4-fluoro-1-(tetrahydro-2H-pyran-2-yl)-1H-pyrazole-3-carboxylate (200 mg, 1 Eq, 876 μmol) in dry THF (3.0 mL) and the mixture was stirred at RT for 18 hours. The reaction was quenched with NH₄Cl (10 mL) and extracted with DCM (2×10 mL). The organic phase was collected, dried (phase separator) and evaporated under reduced pressure, giving (4-fluoro-1-(tetrahydro-2H-pyran-2-yl)-1H-pyrazol-3-yl)methanol (0.20 g) as a clear yellow oil. ¹H NMR (400 MHz, DMSO-d6) δ 7.94 (d, J=4.7 Hz, 1H), 5.27-5.15 (m, 1H), 5.12 (t, J=5.7 Hz, 1H), 4.39 (d, J=5.7 Hz, 2H), 3.97-3.65 (m, 1H), 3.64-3.52 (m, 1H), 2.02 (tdd, J=12.5, 10.0, 3.9 Hz, 1H), 1.94-1.80 (m, 2H), 1.81-1.54 (m, 1H), 1.56-1.35 (m, 2H).

### Step 3

DIPEA (0.15 g, 0.21 mL, 1.5 Eq, 1.2 mmol) was added to a stirred solution of (4-fluoro-1-(tetrahydro-2H-pyran-2-yl)-1H-pyrazol-3-yl)methanol (0.20 g, 80% Wt, 1 Eq, 0.80 mmol) and methanesulfonyl chloride (0.10 g, 68 μL, 1.1 Eq, 0.88 mmol) in dry DCM (6 mL) under a nitrogen atmosphere at 0° C. The reaction mixture was stirred for 18 h at RT. The reaction mixture was diluted with DCM (10 mL) and washed with NaHCO₃ (20 mL); the organic phase was collected, dried (phase separator) and evaporated under reduced pressure, yielding the title compound (0.11 g, 90% Purity) as a dark brown oil. ¹H NMR (400 MHz, DMSO-d6) δ 8.06 (d, J=4.6 Hz, 1H), 5.28 (dd, J=10.1, 2.4 Hz, 1H), 4.72 (s, 2H), 3.95-3.86 (m, 1H), 3.61 (dddd, J=11.5, 8.1, 4.5, 2.0 Hz, 2H), 3.18-3.09 (m, 1H), 2.08-1.96 (m, 1H), 1.96-1.82 (m, 2H), 1.74-1.56 (m, 1H).

### Intermediate 14—3-(chloromethyl)-5-fluoro-1-(tetrahydro-2H-pyran-2-yl)-1H-pyrazole

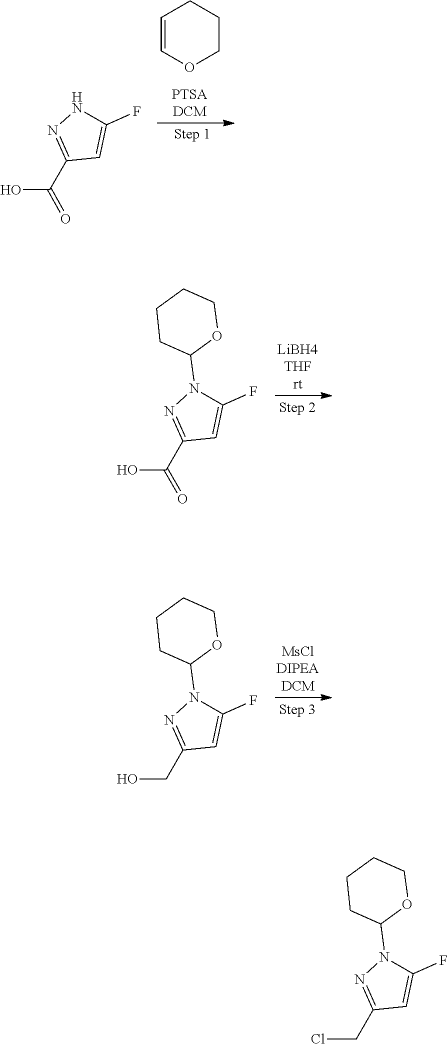 [SMILES: C1=COCCC1 ; O=C(O)c1cc(F)[nH]n1 ; O=C(O)c1cc(F)n(C2CCCCO2)n1 ; OCc1cc(F)n(C2CCCCO2)n1 ; Fc1cc(CCl)nn1C1CCCCO1]

### Step 1

A stirred solution of methyl 4-fluoro-1H-pyrazole-3-carboxylate (120 mg, 1 Eq, 833 μmol) and p-toluenesulfonic acid monohydrate (79.2 mg, 0.5 Eq, 416 μmol) in DCM (12 mL) was treated with 3,4-dihydro-2H-pyran (108 mg, 117 μL, 97% Wt, 1.50 Eq, 1.25 mmol) and the mixture was stirred at RT for 2 h. The reaction mixture was washed with sat. aq. NaHCO₃ (10 mL). The organic layer was collected and the aqueous was extracted with DCM (2×5 mL). The combined organic extracts were dried (phase separator) and concentrated in vacuo to afford methyl 4-fluoro-1-(tetrahydro-2H-pyran-2-yl)-1H-pyrazole-3-carboxylate (0.20 g) as an orange oil. ¹H NMR (400 MHz, DMSO-d6) δ 8.23 (dd, J=4.9, 2.2 Hz, 1H), 5.41 (dd, J=9.6, 2.4 Hz, 1H), 3.96-3.77 (m, 4H), 3.77-3.58 (m, 1H), 2.18-1.96 (m, 1H), 1.96-1.84 (m, 2H), 1.80-1.59 (m, 1H), 1.60-1.26 (m, 2H). MS (ES)⁺: 251 (M+Na)⁺.

### Step 2

Lithium borohydride (38.2 mg, 876 μL, 2.00 molar, 2 Eq, 1.75 mmol) was added dropwise to a stirred solution of methyl 4-fluoro-1-(tetrahydro-2H-pyran-2-yl)-1H-pyrazole-3-carboxylate (200 mg, 1 Eq, 876 μmol) in dry THF (3.0 mL) and the mixture was stirred for 18 hours at RT. The reaction was quenched with NH₄Cl (10 mL) and extracted with DCM (2×10 mL). The organic phase was collected, dried (phase separator) and evaporated under reduced pressure, giving (4-fluoro-1-(tetrahydro-2H-pyran-2-yl)-1H-pyrazol-3-yl)methanol (0.20 g) as a clear yellow oil. The product was used without further purification in the next step. ¹H NMR (400 MHz, DMSO-d6) δ 7.94 (d, J=4.7 Hz, 1H), 5.27-5.15 (m, 1H), 5.12 (t, J=5.7 Hz, 1H), 4.39 (d, J=5.7 Hz, 2H), 3.97-3.65 (m, 1H), 3.64-3.52 (m, 1H), 2.02 (tdd, J=12.5, 10.0, 3.9 Hz, 1H), 1.94-1.80 (m, 2H), 1.81-1.54 (m, 1H), 1.56-1.35 (m, 2H).

### Step 3

DIPEA (48 mg, 65 μL, 1.5 Eq, 0.37 mmol) was added to a stirred solution of (5-fluoro-1-(tetrahydro-2H-pyran-2-yl)-1H-pyrazol-3-yl)methanol (0.10 g, 50% Wt, 1 Eq, 0.25 mmol) and methanesulfonyl chloride (31 mg, 21 μL, 1.1 Eq, 0.27 mmol) in dry DCM (3 mL) under a nitrogen atmosphere at 0° C. The reaction mixture was stirred for 18 h at RT. The reaction mixture was diluted with DCM (10 mL) and washed with NaHCO₃ (20 mL); the organic phase was collected, dried (phase separator) and evaporated under reduced pressure, yielding the title compound (55 mg) as a dark brown oil. ¹H NMR (400 MHz, DMSO-d6) δ 6.19 (d, J=6.0 Hz, 1H), 5.45 (dt, J=9.6, 2.7 Hz, 1H), 4.93-4.81 (m, 2H), 3.88 (d, J=12.0 Hz, 1H), 3.62 (tdd, J=13.1, 10.9, 5.5 Hz, 2H), 3.20-3.07 (m, 2H), 2.09 (tdd, J=13.1, 9.5, 4.1 Hz, 1H), 2.02-1.87 (m, 1H), 1.81 (dq, J=13.0, 3.6 Hz, 1H).

### Intermediate 15—5-(1-chloroethyl)-2-methoxypyridine hydrochloride

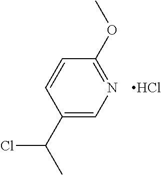

Thionyl chloride (188 mg, 115 μL, 1.1 Eq, 1.58 mmol) was added to a solution of 1-(6-methoxypyridin-3-yl)ethan-1-ol (220 mg, 1 Eq, 1.44 mmol) in toluene (5 mL) and the mixture was stirred for 1.5 h, then concentrated in vacuo. The residue was triturated with MTBE and the resulting mixture was concentrated in vacuo to afford the crude title compound as a colourless semi-solid. The product was used without purification in the next step. Quantitative yield was assumed.

### Intermediate 16—methyl 3-(bromomethyl)picolinate

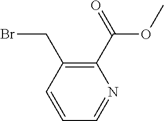

A solution of methyl 3-methylpicolinate (1.51 g, 10.0 mmol), N-bromosuccinimide (1.6 g, 9.0 mmol), AIBN (164 mg, 1.0 mmol) in CCl₄ (50 mL) was stirred at 75° C. for 12 h. The mixture was diluted with sat. NaHCO₃, extracted with DCM, and washed with brine (10 mL). The organic layer was dried (MgSO₄), filtered and concentrated at 40° C. under reduced pressure. The residue was purified by flash column chromatography to give the title compound (1.0 g, 44%) as a yellow oil. ¹H NMR (400 MHz, CDCl3) δ: 8.67 (dd, J1=2.0 Hz, J2=4.8 Hz, 1H), 7.90 (dd, J1=1.6 Hz, J2=8.0 Hz, 1H), 7.48 (dd, J1=4.8 Hz, J2=8.0 Hz, 1H), 4.94 (s, 3H).

### Intermediate 17—methyl 3-(bromomethyl)thiophene-2-carboxylate

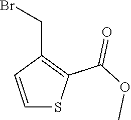

To a solution of methyl 3-methylthiophene-2-carboxylate (500 mg, 3.20 mmol), in CCl₄ (10 mL) was added NBS (456 mg, 2.56 mmol) and AIBN (53 mg, 0.32 mmol), and the mixture was stirred at 75° C. for 12 h. The mixture was diluted with sat. NaHCO₃, extracted with DCM, and washed with brine (10 mL). The organic layer was dried (MgSO₄), filtered and concentrated at 40° C. under reduced pressure. The residue was purified by flash column chromatography to give the title compound (300 mg) as a yellow oil. MS (ES⁺): 235.0 (M+H)⁺.

### Intermediate 18—methyl 3-(bromomethyl)furan-2-carboxylate

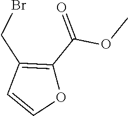

To a solution of methyl 3-methylfuran-2-carboxylate (500 mg, 3.57 mmol), was added NBS (509 mg, 2.86 mmol), AIBN (60 mg, 0.36 mmol) in CCl₄ (10 mL). Then mixture was stirred at 75° C. for 12 h. The mixture was diluted with sat. aq. NaHCO₃, extracted with DCM, and washed with brine (10 mL). The organic layer was dried (MgSO₄), filtered and concentrated at 40° C. under reduced pressure. The residue was purified by flash column chromatography to give the title compound (300 mg, 39%) as a yellow oil. MS (ES⁺): 219.0 (M+H)⁺.

### Intermediate 19—methyl 3-(bromomethyl)-6-methoxypicolinate

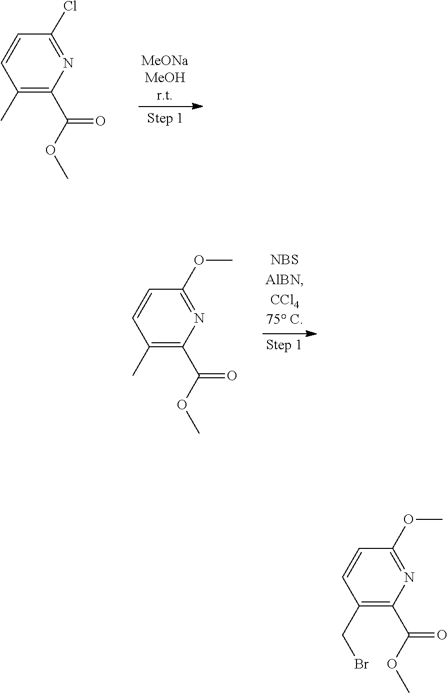

### Step 1

A solution of methyl 6-chloro-3-methylpicolinate (900 mg, 4.86 mmol), MeONa (2M in MeOH) (5 mL) in MeOH (2 mL). Then mixture was stirred at RT for 12 h. The mixture was diluted with saturated NH₄Cl, extracted with DCM, and washed with brine (5 mL). The organic layer was dried (MgSO₄), filtered and concentrated at 40° C. under reduced pressure. The residue was purified by flash column chromatography to give methyl 6-methoxy-3-methylpicolinate (200 mg, 23%) as a yellow oil. MS (ES⁺): 182.3 (M+H)⁺.

### Step 2

A solution of methyl 6-methoxy-3-methylpicolinate (200 mg, 1.10 mmol), NBS (157 mg, 0.88 mmol), AIBN (18 mg, 0.11 mmol) in CCl₄ (5 mL). The mixture was stirred at 75° C. for 12 hours. The mixture was diluted with sat. NaHCO₃, extracted with DCM, and washed with brine (5 mL). The organic layer was dried (MgSO₄), filtered and concentrated at 40° C. under reduced pressure. The residue was purified by flash column chromatography to give the title compound (200 mg) as a yellow oil. MS (ES⁺): 260.0 (M+H)⁺.

### Intermediate 20—methyl 5-(bromomethyl)furan-2-carboxylate

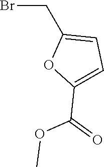

A solution of methyl 5-methylfuran-2-carboxylate (500 mg, 3.57 mmol), NBS (509 mg, 2.86 mmol), AIBN (59 mg, 0.36 mmol) in CCl₄ (10 mL). Then mixture was stirred at 75° C. for 12 h. The mixture was diluted with sat. NaHCO₃, extracted with DCM, and washed with brine (10 mL). The organic layer was dried (MgSO₄), filtered and concentrated at 40° C. under reduced pressure. The residue was purified by flash column chromatography to give the title compound (300 mg) as a yellow oil. MS (ES⁺): 219.0 (M+H)⁺.

### Intermediate 21—2-(chloromethyl)-5-methoxypyrazine

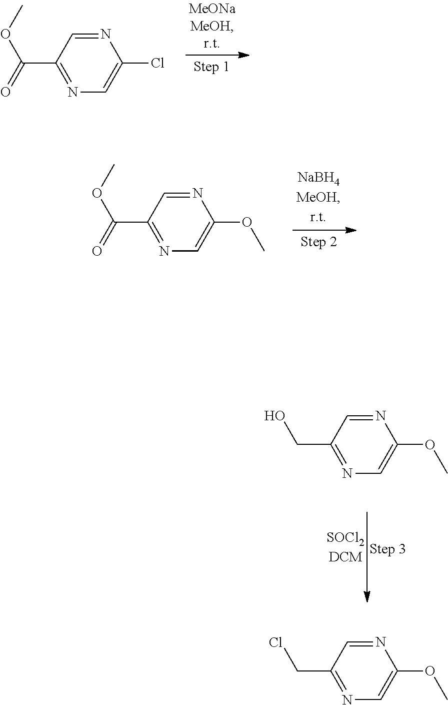

### Step 1

To the solution of methyl 5-chloropyrazine-2-carboxylate (1 g, 5.8 mmol) in MeOH (30 mL) was added MeONa (344 mg, 6.4 mmol) at room temperature, and the reaction mixture was stirred at RT for 3 h. After LCMS indicated the reaction was complete, the reaction mixture was extracted with EtOAc (2×50 mL). The organic layer was washed with brine, dried over Na₂SO₄, filtered and concentrated at 30° C. under reduced pressure and the residue was purified by flash column chromatography to give methyl 5-methoxypyrazine-2-carboxylate (400 mg) as a yellow oil. MS (ES⁺): 169.2 (M+H)⁺.

### Step 2

To the solution of methyl 5-methoxypyrazine-2-carboxylate (400 mg, 2.4 mmol) in MeOH (12 mL) was NaBH₄ (880 mg, 24 mmol) at 0° C., and the reaction mixture was stirred at RT for 1 h. After LCMS indicated the reaction was complete, the reaction mixture was quenched with ice water (10 mL), adjusted to pH=5 with 0.5N HCl aqueous solution, and extracted with EtOAc (2×50 mL). The organic layer was washed with brine, dried over Na₂SO₄, filtered and concentrated at 30° C. under reduced pressure and the residue was purified by flash column chromatography to give (5-methoxypyrazin-2-yl)methanol (300 mg) as a yellow oil. MS (ES⁺): 141.3 (M+H)⁺.

### Step 3

To the solution of (5-methoxypyrazin-2-yl)methanol (300 mg, 2.1 mmol) in DCM (8 mL) was added drop-wise SOCl₂ (1.25 g, 10.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×10 mL). The organic layer was washed with brine, dried over Na₂SO₄, filtered and concentrated at 30° C. under reduced pressure to afford the title compound (250 mg) as a yellow oil, which was used to the next step directly. MS (ES⁺): 159.2 (M+H)⁺.

### Intermediate 22—methyl 2-(bromomethyl)-6-chlorobenzoate

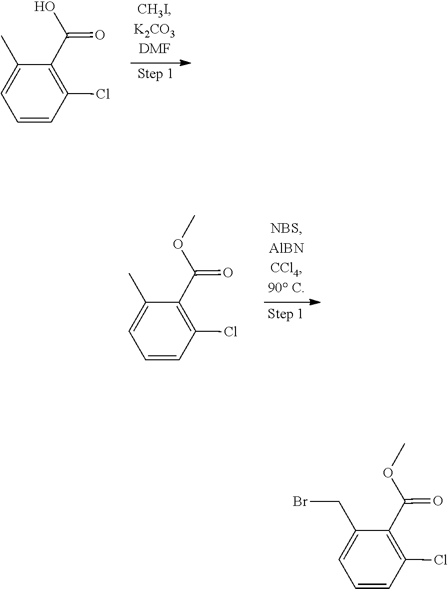

### Step 1

To the solution of ethyl 2-chloro-6-methylbenzoic acid (1.5 g, 8.8 mmol) and CH₃I (1.3 g, 9 mmol) in DMF (50 mL) was added K₂CO₃ (2.4 mg, 17 mmol) at RT, and the reaction mixture was stirred at RT for 4 h. After LCMS indicated the reaction was complete, the reaction mixture was extracted with EtOAc (2×50 mL). The organic layer was washed with brine, dried over Na₂SO₄, filtered and concentrated at 30° C. under reduced pressure and the residue was purified by flash column chromatography to give methyl 2-chloro-6-methylbenzoate (1.5 g) as a yellow oil. ¹H NMR (400 MHz, DMSO-d6) δ: 7.22 (t, J=3.6 Hz, 2H), 7.11 (t, J=5.2 Hz, 1H), 3.95 (s, 3H), 2.32 (s, 3H).

### Step 2

To the solution of methyl 2-chloro-6-methylbenzoate (1.5 g, 8 mmol) and NBS (1.56 g, 8.8 mmol) in CCl₄ (50 mL) was added AIBN (263 mg, 1.6 mmol) at RT, and the reaction mixture was stirred at 90° C. for 4 hours. After LCMS indicated the reaction was complete, the reaction mixture was extracted with EtOAc (2×50 mL). The organic layer was washed with brine, dried over Na₂SO₄, filtered and concentrated at 30° C. under reduced pressure and the residue was purified by flash column chromatography to give the title compound (1.1 g) as a yellow oil. MS (ES⁺): 263.0 (M+H)⁺.

### Intermediate 23—ethyl 2-(bromomethyl)-6-methoxybenzoate

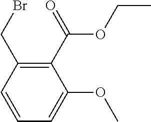

To the solution of ethyl 2-methoxy-6-methylbenzoate (2 g, 10 mmol) and NBS (1.95 g, 11 mmol) in CCl₄ (50 mL) was added AIBN (328 mg, 2 mmol) at RT, and the reaction mixture was stirred at 90° C. for 4 h. After LCMS indicated the reaction was complete, the reaction mixture was extracted with EtOAc (2×50 mL). The organic layer was washed with brine, dried over Na₂SO₄, filtered and concentrated at 30° C. under reduced pressure and the residue was purified by flash column chromatography to give the title compound (1.8 g) as a yellow oil. MS (ES⁺): 273.0 (M+H)⁺.

### Intermediate 24—methyl 4-(bromomethyl)thiazole-2-carboxylate

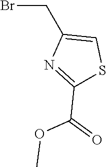

To a solution of methyl 4-methylthiazole-2-carboxylate (900 mg, 5.73 mmol) in CCl₄ (10 mL) was added NBS (815 mg, 4.58 mmol) and AIBN (94 mg, 0.57 mmol), and then mixture was stirred at 75° C. for 12 h. The mixture was diluted with sat. aq. NaHCO₃, extracted with DCM, and washed with brine (10 mL). The organic layer was dried (MgSO₄), filtered and concentrated at 40° C. under reduced pressure. The residue was purified by flash column chromatography to give the title compound (300 mg) as a yellow oil. MS (ES⁺): 236.0 (M+H)⁺.

### Intermediate 25—methyl 4-(chloromethyl)-5-methylisoxazole-3-carboxylate

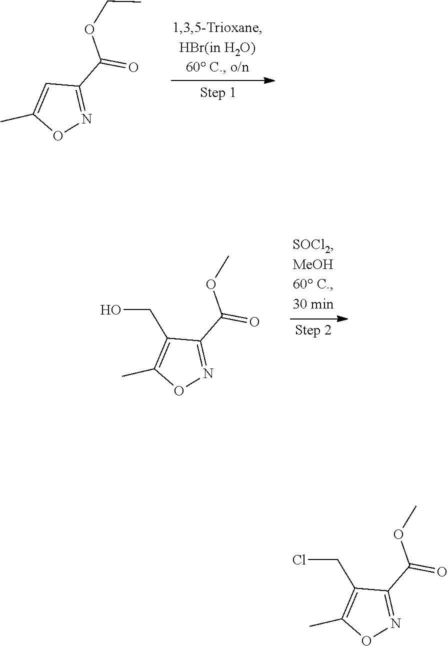

### Step 1

A mixture of the ethyl 5-methylisoxazole-3-carboxylate (900 mg, 5.80 mmol)), 1,3,5-trioxane (783 mg, 8.70 mmol) and aqueous HBr (62%, 10.0 mL) was stirred in a sealed flask at 60° C. overnight. After cooling the mixture was poured into absolute MeOH (20 mL) and evaporated at 60˜70° C. The mixture was used crude into the next step without further manipulation.

### Step 2

The mixture was dissolved in MeOH (20 ml) and thionyl chloride (4 mL) added; then the mixture was stirred at 60° C. for 30 min. The reaction mixture was dried, then evaporated to afford the title compound (300 mg) as a yellow oil. MS (ES⁺): 190.2 (M+H)⁺.

### Intermediate 26—2-chloro-3-(chloromethyl)-6-methylpyridine

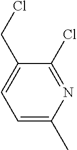

To a mixture of (2-chloro-6-methylpyridin-3-yl)methanol (400 mg, 2.5 mmol) in DCM (5 mL) was added SOCl₂ (1.1 g, 9.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×10 mL). The organic layer was washed with brine, dried over Na₂SO₄, filtered and concentrated at 30° C. under reduced pressure to give the title compound (400 mg) as a yellow oil, which was used to the next step directly. MS (ES⁺): 176.2 (M+H)⁺.

### Intermediate 27—2-chloro-3-(chloromethyl)-4-methylpyridine

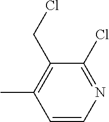

To a mixture of (2-chloro-4-methylpyridin-3-yl)methanol (300 mg, 1.9 mmol) in DCM (5 mL) was added SOCl₂ (1.1 g, 9.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×10 mL). The organic layer was washed with brine, dried over Na₂SO₄, filtered and concentrated at 30° C. under reduced pressure to give the title compound (300 mg) as a yellow oil, which was used to the next step directly. MS (ES⁺): 176.2 (M+H)⁺.

### Intermediate 28—2-chloro-3-(chloromethyl)-5-fluoropyridine

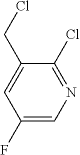

To a mixture of (2-chloro-5-fluoropyridin-3-yl)methanol (500 mg, 3.1 mmol) in DCM (5 mL) was added SOCl₂ (1.1 g, 9.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (10 mL×2). The organic layer was washed with brine, dried over Na₂SO₄, filtered and concentrated at 30° C. under reduced pressure to give the title compound (400 mg) as a yellow oil, which was used to the next step directly. ¹H NMR (400 MHz, CDCl3) δ: 8.24 (d, J=2.8 Hz, 1H), 7.66 (dd, J=8.0, 2.8 Hz, 1H), 4.67 (s, 2H).

### Intermediate 29—2-chloro-3-(chloromethyl)-5-isopropylpyridine

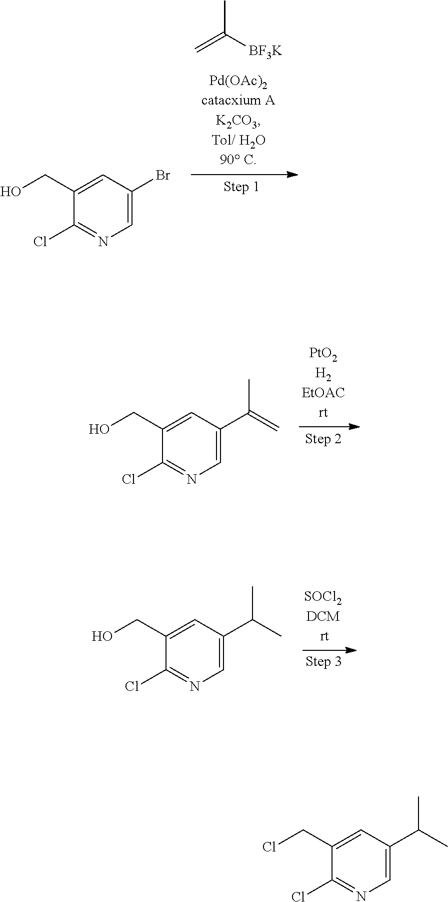

### Step 1

To a mixture of (5-bromo-2-chloropyridin-3-yl)methanol (1.4 g, 6.3 mmol), Pd(Oac)₂ (141 mg, 0.63 mmol), catacxium A (443 mg, 1.2 mmol), K₂CO₃ (1.7 g, 12 mmol) in Tol/H₂O (10 mL, 5:1) was added potassium isopropenyltrifluoroborate (962 mg, 6.5 mmol) at RT, and the reaction mixture was stirred at 90° C. overnight under nitrogen atmosphere. After LCMS indicated the reaction was complete, the reaction mixture was filtered through Celite and washed with EtOAc. The filtrate was diluted with water (10 mL) and extracted with EtOAc (3×10 mL). The combined organic layer was washed by brine, dried over Na₂SO₄, filtered and concentrated at 45° C. under reduced pressure. The residue was purified by flash column chromatography to give (2-chloro-5-(prop-1-en-2-yl)pyridin-3-yl)methanol (1.1 g) as a yellow solid. MS (ES⁺): 184.2 (M+H)⁺.

### Step 2

To a solution of (2-chloro-5-(prop-1-en-2-yl)pyridin-3-yl)methanol (1.1 g, 6 mmol) in EtOAc (10 mL) was added PtO₂ (1.4 g, 6.6 mmol) at RT, and the reaction mixture was stirred at room temperature for 2 h under H₂. After LCMS indicated the reaction was complete, the reaction mixture was filtered through Celite and washed with EtOAc. The filtrate was diluted with water (10 mL) and extracted with EtOAc (3×15 mL). The combined organic layer was washed with brine, dried over Na₂SO₄, filtered and concentrated at 45° C. under reduced pressure. The residue was purified by flash column chromatography to give (2-chloro-5-isopropylpyridin-3-yl)methanol (1 g) as yellow solid. MS (ES⁺): 186.2 (M+H)⁺.

### Step 3

To a mixture of (2-chloro-5-isopropylpyridin-3-yl)methanol (1 g, 5.4 mmol) in DCM (15 mL) was added SOCl₂ (2.4 g, 20 mmol) at 0° C., and the reaction mixture was stirred at RT for 3 h. After LCMS indicated the reaction is completed, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×20 mL). The organic layer was washed with brine, dried over Na₂SO₄, filtered and concentrated at 30° C. under reduced pressure to give 2-chloro-3-(chloromethyl)-5-isopropylpyridine (800 mg) as a yellow oil. Used without further purification. MS (ES⁺): 204.2 (M+H)⁺.

### Intermediate 30—1-(2-(tert-butyldimethylsilyloxy)ethyl)-3-(chloromethyl)pyridin-2(1H)-one

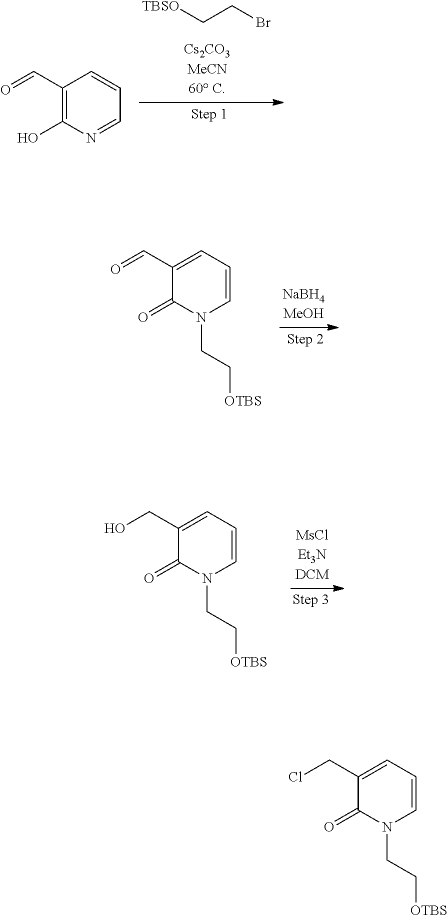 [SMILES: CC(C)(C)[Si](C)(C)OCCBr ; O=Cc1cccnc1O ; CC(C)(C)[Si](C)(C)OCCn1cccc(C=O)c1=O ; CC(C)(C)[Si](C)(C)OCCn1cccc(CO)c1=O ; CC(C)(C)[Si](C)(C)OCCn1cccc(CCl)c1=O]

### Step 1

To the solution of 2-hydroxynicotinaldehyde (200 mg, 1.6 mmol), Cs₂CO₃ (1.56 g, 4.8 mmol) in MeCN (15 mL) was added (2-bromoethoxy)(tert-butyl)dimethylsilane (571 mg, 2.4 mmol) at 60° C. overnight. After LCMS indicated the reaction completed, the reaction mixture was quenched with water (10 mL) and filtered. The cake was dissolved ethyl acetate (10 mL), and washed with brine, dried over Na₂SO₄ and filtered. The filtrate was concentrated at 30° C. under reduced pressure, the residue was triturated with petroleum ether/ethyl acetate=1/1, filtered and dried at 30° C. under reduced pressure to give 1-(2-(tert-butyldimethylsilyloxy)ethyl)-2-oxo-1,2-dihydropyridine-3-carbaldehyde (200 mg) as a white solid. MS (ES⁺): 282.2 (M+H)⁺.

### Step 2

To the solution of 1-(2-(tert-butyldimethylsilyloxy)ethyl)-2-oxo-1,2-dihydropyridine-3-carbaldehyde (200 mg, 0.71 mmol) in MeOH (10 mL) was add NaBH₄ (54 mg, 1.42 mmol) at 0° C., and the reaction mixture was stirred at RT for 3 h. After LCMS indicated the reaction was complete, the mixture was concentrated at 30° C. under reduced pressure to remove DCM. The residue was quenched with ice water (10 mL), and extracted with DCM (2×10 mL). The organic layer was washed with brine, dried over Na₂SO₄, filtered and concentrated at 30° C. under reduced pressure to give 1-(2-(tert-butyldimethylsilyloxy)ethyl)-3-(hydroxymethyl)pyridin-2(1H)-one (120 mg) as a yellow oil. MS (ES⁺): 284.2 (M+H)⁺.

### Step 3

To the solution of 1-(2-(tert-butyldimethylsilyloxy)ethyl)-3-(hydroxymethyl)pyridin-2(1H)-one (120 mg, 0.4 mmol), Et₃N (121 mg, 1.2 mmol) in DCM (5 mL) was added MsCl (68 mg, 0.6 mmol) at RT for 3 h. After LCMS indicated the reaction completed, the reaction mixture was quenched with water (10 mL) and filtered. The cake was dissolved DCM (5 mL), and washed with brine, dried over Na₂SO₄ and filtered. The filtrate was concentrated at 30° C. under reduced pressure to give 1-(2-(tert-butyldimethylsilyloxy)ethyl)-3-(chloromethyl)pyridin-2(1H)-one (80 mg) as a yellow oil. MS (ES⁺): 302.2 (M+H)⁺.

### Intermediate 31—2,4-dichloro-3-(chloromethyl)pyridine

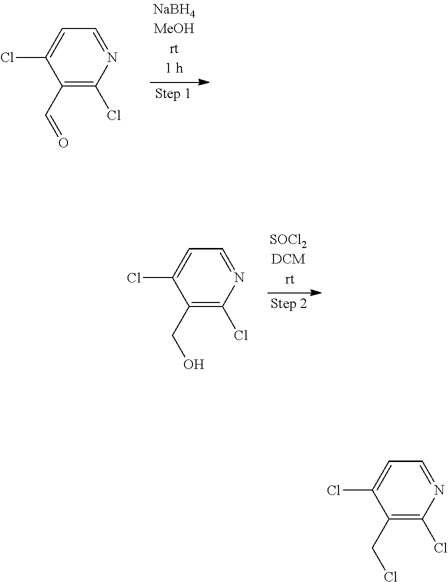 [SMILES: O=Cc1c(Cl)ccnc1Cl ; OCc1c(Cl)ccnc1Cl ; ClCc1c(Cl)ccnc1Cl]

To the solution of 2,4-dichloronicotinaldehyde (1.0 g, 5.71 mmol) in MeOH (20 mL) was add NaBH₄ (433 mg, 11.4 mmol) at 0° C., and the reaction mixture was stirred at RT for 1 h. After LCMS indicated the reaction was complete, the mixture was concentrated at 30° C. under reduced pressure to remove DCM. The residue was quenched with ice water (10 mL), and extracted with DCM (2×10 mL). The organic layer was washed with brine, dried over Na₂SO₄, filtered and concentrated at 30° C. under reduced pressure to give (2,4-dichloropyridin-3-yl)methanol (900 mg) as a yellow oil. MS (ES⁺): 178.2 (M+H)⁺.

### Step 2

To the solution of (2,4-dichloropyridin-3-yl)methanol (900 mg, 5.1 mmol) in DCM (10 mL) was added drop-wise SOCl₂ (3 g, 25.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (50 mL), adjusted to pH=9 with 2N K₂CO₃ aqueous solution, and extracted with DCM (2×40 mL). The organic layer was washed with brine, dried over Na₂SO₄, filtered and concentrated at 30° C. under reduced pressure to give the title compound (800 mg) as a yellow oil. MS (ES⁺): 196.0 (M+H)⁺.

### Intermediate 32—2-chloro-3-(chloromethyl)-4-fluoropyridine

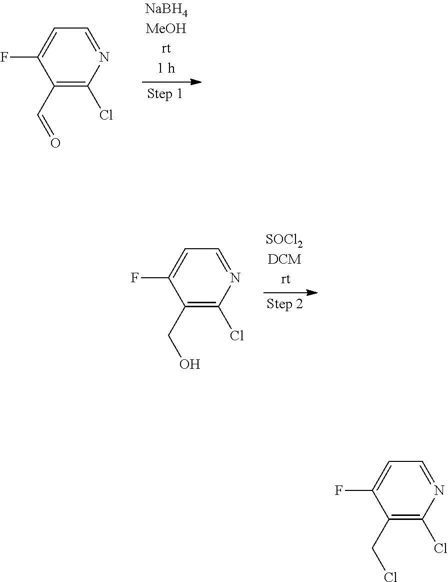 [SMILES: O=Cc1c(F)ccnc1Cl ; OCc1c(F)ccnc1Cl ; Fc1ccnc(Cl)c1CCl]

### Step 1

To the solution of 2-chloro-4-fluoronicotinaldehyde (908 mg, 5.71 mmol) in MeOH (20 mL) was add NaBH₄ (433 mg, 11.4 mmol) at 0° C., and the reaction mixture was stirred at RT for 1 h. After LCMS indicated the reaction was complete, the mixture was concentrated at 30° C. under reduced pressure to remove DCM. The residue was quenched with ice water (10 mL) and extracted with DCM (2×10 mL). The organic layer was washed with brine, dried over Na₂SO₄, filtered and concentrated at 30° C. under reduced pressure to give (2-chloro-4-fluoropyridin-3-yl)methanol (820 mg) as a yellow oil. MS (ES⁺): 162.2 (M+H)⁺.

### Step 2

To a solution of (2-chloro-4-fluoropyridin-3-yl)methanol (820 mg, 5.1 mmol) in DCM (10 mL) SOCl₂ (3 g, 25.5 mmol) was added drop-wise at 0° C., and the reaction mixture was stirred at RT for 3 hours. After LCMS indicated the reaction was complete, the mixture was concentrated at 30° C. under reduced pressure. The residue was quenched with ice water (50 mL), adjusted to pH=9 with 2N K₂CO₃ aqueous solution, and extracted with DCM (2×40 mL). The organic layer was washed with brine, dried over Na₂SO₄, filtered and concentrated at 30° C. under reduced pressure to give the title compound (700 mg) as a yellow oil. MS (ES⁺): 180.0 (M+H)⁺.

### Intermediate 33—3-bromo-4-(chloromethyl)pyridine

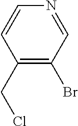

To a mixture of (3-bromopyridin-4-yl)methanol (300 mg, 1.6 mmol) in DCM (5 mL) was added SOCl₂ (1.1 g, 9.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×10 mL). The organic layer was washed with brine, dried over Na₂SO₄, filtered and concentrated at 30° C. under reduced pressure to afford the title compound (305 mg) as a yellow oil. MS (ES⁺): 206.0 (M+H)⁺.

### Intermediate 34—methyl 2-(bromomethyl)-5-methoxybenzoate

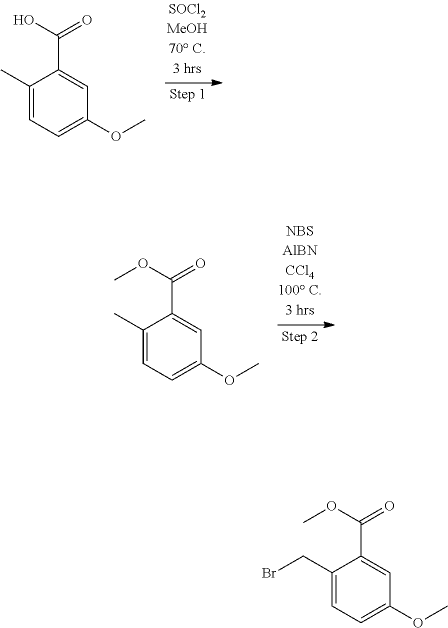

### Step 1

To the solution of 5-methoxy-2-methylbenzoic acid (1.0 g, 6 mmol) in MeOH (20 mL) was added SOCl₂ (1.4 mg, 12 mmol) at RT, and the reaction mixture was stirred at 70° C. for 3 h. After LCMS indicated the reaction is completed, the reaction mixture was extracted with EtOAc (2×20 mL). The organic layer was washed with brine, dried over Na₂SO₄, filtered and concentrated at 30° C. under reduced pressure and the residue was purified by flash column chromatography to give methyl 5-methoxy-2-methylbenzoate (1.1 g) as a yellow oil. MS (ES⁺): 181.2 (M+H)⁺.

### Step 2

To the solution of methyl 5-methoxy-2-methylbenzoate (1.1 g, 6 mmol) and NBS (1.17 g, 6.6 mmol) in CCl₄ (30 mL) was added AIBN (197 mg, 1.2 mmol) at RT, and the reaction mixture was stirred at 100° C. for 3 h. After LCMS indicated the reaction was complete, the reaction mixture was extracted with EtOAc (2×30 mL). The organic layer was washed with brine, dried over Na₂SO₄, filtered and concentrated at 30° C. under reduced pressure. The residue was purified by flash column chromatography to give the title compound (700 mg) as a yellow oil. ¹H NMR (400 MHz, CDCl3) δ: 7.48 (d, J=2.8 Hz, 1H), 7.36 (dd, J=9.2, 2.8 Hz, 1H), 7.02 (dd, J=8.4, 2.8 Hz, 1H), 4.94 (s, 2H), 3.95 (s, 3H), 3.85 (s, 3H).

### Intermediate 35—4-chloro-3-(chloromethyl)pyridine

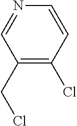

To a mixture of (4-chloropyridin-3-yl)methanol (500 mg, 3.5 mmol) in DCM (5 mL) was added SOCl₂ (1.7 g, 14 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (2×10 mL). The organic layer was washed with brine, dried over Na₂SO₄, filtered and concentrated at 30° C. under reduced pressure to give the title compound (490 mg) as a yellow oil. MS (ES⁺): 162.2 (M+H)⁺.

### Intermediate 36—2-chloro-3-(chloromethyl)-6-methoxypyridine

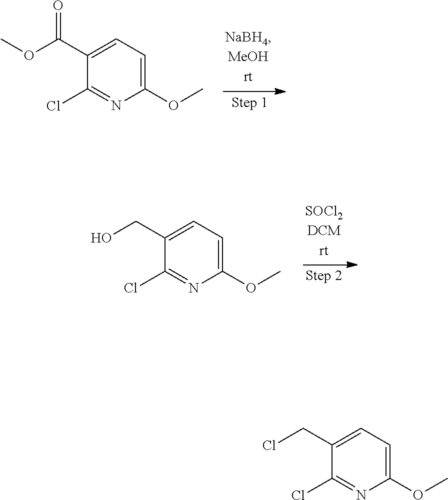

### Step 1

To the solution of methyl 2-chloro-6-methoxynicotinate (500 mg, 2.49 mmol) in MeOH (10 mL) was add NaBH₄ (189 mg, 4.98 mmol) at 0° C., and the reaction mixture was stirred at RT for 1 h. After LCMS indicated the reaction was complete, the mixture was concentrated at 30° C. under reduced pressure to remove DCM. The residue was quenched with ice water (10 mL), and extracted with DCM (2×10 mL). The organic layer was washed with brine, dried over Na₂SO₄, filtered and concentrated at 30° C. under reduced pressure to give (2-chloro-6-methoxypyridin-3-yl)methanol (400 mg) as a yellow oil. MS (ES⁺): 174.2 (M+H)⁺.

### Step 2

To the solution of (2-chloro-6-methoxypyridin-3-yl)methanol (400 mg, 2.3 mmol) in DCM (10 mL) was drop-wised SOCl₂ (1.4 g, 11.5 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (50 mL), adjusted to pH=9 with 2N K₂CO₃ aqueous solution, and extracted with DCM (2×40 mL). The organic layer was washed with brine, dried over Na₂SO₄, filtered and concentrated at 30° C. under reduced pressure to give the title compound (380 mg) as a yellow oil. MS (ES⁺): 192.0 (M+H)⁺.

### Intermediate 37—5-(chloromethyl)-3-(trifluoromethyl)isoxazole

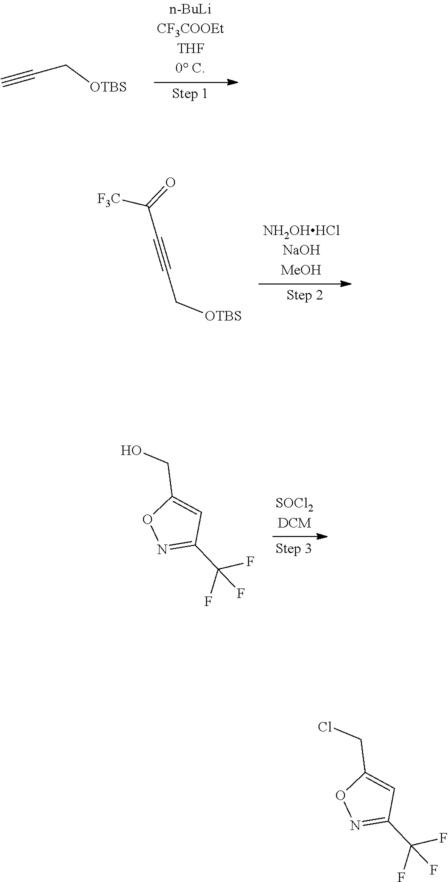 [SMILES: C#CCO[Si](C)(C)C(C)(C)C ; CC(C)(C)[Si](C)(C)OCC#CC(=O)C(F)(F)F ; OCc1cc(C(F)(F)F)no1 ; FC(F)(F)c1cc(CCl)on1]

### Step 1

To 50 mL of a 2.5M solution of n-BuLi (30 mL, 76.44 mmol) in hexanes was added dropwise tert-butyldimethyl(prop-2-ynyloxy)silane (10 g, 58.8 mmol) in ice THF (50 mL). The mixture was stirred at 10° C. for 15 min. To the resulting slightly yellow solution was added ethyl 2,2,2-trifluoroacetate (11.7 g, 82.3 mmol) with cooling in a dry ice acetone bath (−40° C.). The mixture was stirred at RT for 18 h. The reaction mixture was poured onto ice mixed with 10% aqueous citric acid. The phases were separated and the organic phase was washed with 10% aqueous sodium bicarbonate and brine and purified by filtration over ca 120 g silica gel, filtered and dried at 30° C. under reduced pressure to give 5-(tert-butyldimethylsilyloxy)-1,1,1-trifluoropent-3-yn-2-one (5.8 g) as a yellow oil, which was used to the next step directly.

### Step 2

To a solution of hydroxylamine hydrochloride (1.5 g, 21.8 mmol) and sodium hydroxide (43.6 mg, 1.09 mmol) in 25 mL methanol was added 5-(tert-butyldimethylsilyloxy)-1,1,1-trifluoropent-3-yn-2-one (5.8 g, 21.8 mmol) and the mixture was heated to reflux for 3 h. The reaction mixture was extracted with EtOAc and purified by chromatography on silica gel to give (3-(trifluoromethyl)isoxazol-5-yl)methanol (1.5 g as colourless oil. ¹H NMR (400 MHz, DMSO-d6) δ: 8.39 (s, 1H), 5.46-5.42 (m, 1H), 4.20 (d, J=5.6 Hz, 2H). ¹⁹F NMR (376.5 MHz, DMSO-d₆) δ: −81.99.

### Step 3

To the solution of (3-(trifluoromethyl)isoxazol-5-yl)methanol (200 mg 1.20 mmol) in DCM (5 mL) was added drop-wise SOCl₂ (714 mg, 6.0 mmol) at 0° C., and the reaction mixture was stirred at RT for 3 h. After LCMS indicated the reaction was complete, the mixture was concentrated at 30° C. under reduced pressure to remove DCM and most of the SOCl₂ to give the title compound (185 mg) as a yellow oil, which was used in the next step without further characterisation or purification.

### Intermediate 38—3-bromo-2-(chloromethyl)pyridine

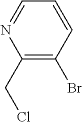

To a mixture of (3-bromopyridin-2-yl)methanol (500 mg, 2.7 mmol) in DCM (5 mL) was added SOCl₂ (1.7 g, 14 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (10 mL), adjusted to pH=9 with 2N K₂CO₃ aqueous solution, and extracted with DCM (10 mL×2). The organic layer was washed with brine, dried over Na₂SO₄, filtered and concentrated at 30° C. under reduced pressure to give the title compound (490 mg) as a yellow oil, which was used to the next step directly. MS (ES⁺): 206.0 (M+H)⁺

### Intermediate 39—6-bromo-2-((6-methoxypyridin-3-yl)methyl)phthalazin-1(2H)-one

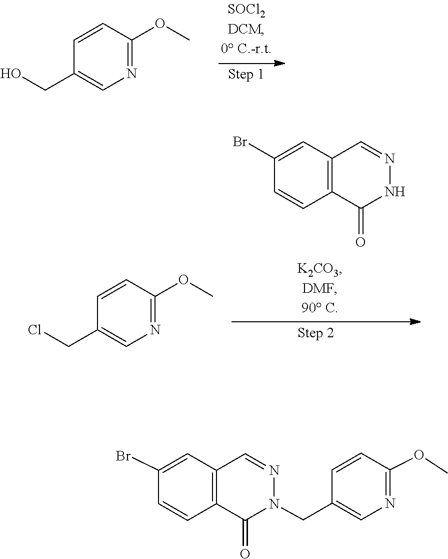

### Step 1

To the solution of (6-methoxypyridin-3-yl)methanol (22.8 g, 164 mmol) in DCM (80 mL) was added SOCl₂ (58.5 g, 492 mmol) at 0° C., and the reaction mixture was stirred at RT for 3 h. After LCMS indicated the reaction was complete, the mixture was concentrated at 30° C. under reduced pressure. The mixture was added ice water (50 mL). the reaction mixture was quenched with 2N K₂CO₃ aqueous to pH=9, separated and extracted with DCM (40 mL×2), dried over Na₂SO₄ and filtered. The filtrate was concentrated at 30° C. under reduced pressure to give 5-(chloromethyl)-2-methoxypyridine (25 g, 97%) as a yellow oil. MS (ES⁺): 158.2 (M+H)⁺

### Step 2

To the solution of 5-(chloromethyl)-2-methoxypyridine (25 g, 159 mmol), K₂CO₃ (33.8 g, 245 mmol) in DMF (80 mL) was added 6-bromophthalazin-1(2H)-one (27.3 g, 122 mmol) at RT, and the reaction mixture was stirred at 90° C. overnight. After LCMS indicated the reaction was complete, the reaction mixture was quenched with water (300 mL) and filtered. The cake was dissolved in DCM (400 mL), and washed with brine, dried over Na₂SO₄ and filtered. The filtrate was concentrated at 30° C. under reduced pressure and the resulting residue was then triturated with hexane and MTBE (10/1), filtered and dried at 30° C. under reduced pressure to give the title compound (52 g) as a yellow solid. MS (ES⁺): 346.0 (M+H)⁺

### Intermediate 40—6-mercapto-2-((6-methoxypyridin-3-yl)methyl)phthalazin-1(2H)-one

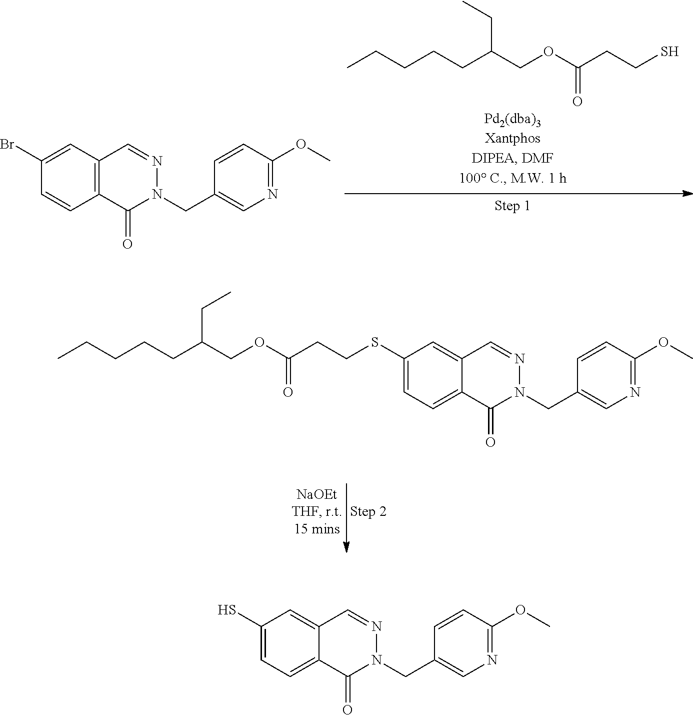 [SMILES: CCCCCC(CC)COC(=O)CCS ; COc1ccc(Cn2ncc3cc(Br)ccc3c2=O)cn1 ; CCCCCC(CC)COC(=O)CCSc1ccc2c(=O)n(Cc3ccc(OC)nc3)ncc2c1 ; COc1ccc(Cn2ncc3cc(S)ccc3c2=O)cn1]

### Step 1

To the solution of 6-bromo-2-((6-methoxypyridin-3-yl)methyl)phthalazin-1(2H)-one (Intermediate 39, 52 g, 150 mmol), Pd₂(dba)₃ (13.73 g, 15 mmol), Xantphos (17.34 g, 30 mmol), DIPEA (58 g, 450 mmol) in DMF (80 mL) was added 2-ethylhexyl 3-mercaptopropanoate (49 g, 225 mmol) at RTroom temperature under N₂, and the reaction mixture was stirred at 100° C. overnight. After LCMS indicated the reaction was complete, the reaction mixture was filtered. The mixture was added EtOAc (80 mL), the combined organic layer was washed by brine, separated and back-extracted with more EtOAc (50 mL×3). Organic phase dried over Na₂SO₄, filtered, and concentrated at 45° C. under reduced pressure; the residue was purified by flash column chromatography to give 6-bromo-2-((6-methoxypyridin-3-yl)methyl)phthalazin-1(2H)-one (62 g, 86%) as a yellow solid. MS (ES⁺): 484.0 (M+H)⁺

### Step 2

A solution of 6-bromo-2-((6-methoxypyridin-3-yl)methyl)phthalazin-1(2H)-one (5 g, 10 mmol) in THF (50 mL) was added NaOEt (816 mg, 12 mmol) at RT. The reaction mixture was stirred at RT for 15 mins. After LCMS indicated the reaction completed. the reaction mixture was quenched with 0.5 N HCl aqueous to pH=5, separated and extracted with EtOAc (20 mL×2). The combined organic layer was washed with brine, dried over Na₂SO₄, filtered and concentrated at 40° C. under reduced pressure to give the title compound (2.6 g as brown solid. MS (ES⁺): 300.0 (M+H)⁺

### Intermediate 41—tert-butyl (3-((6-bromo-1-oxophthalazin-2(1H)-yl)methyl)phenyl)carbamate

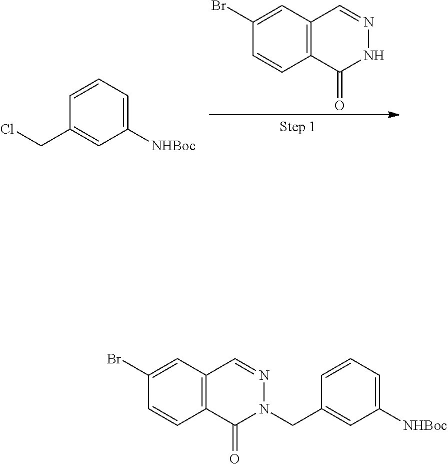 [SMILES: CC(C)(C)OC(=O)Nc1cccc(CCl)c1 ; O=c1[nH]ncc2cc(Br)ccc12 ; CC(C)(C)OC(=O)Nc1cccc(Cn2ncc3cc(Br)ccc3c2=O)c1]

tert-Butyl (3-(bromomethyl)phenyl)carbamate (5.1 g, 1 Eq, 18 mmol) was added to a stirred suspension of 6-bromophthalazin-1(2H)-one (4.0 g, 1.0 Eq, 18 mmol) and cesium carbonate (12 g, 2.0 Eq, 36 mmol) in dry DMF (100 mL) under a N₂ at 70° C. The reaction mixture was stirred for 18 h. The product was precipitated with water (500 mL) and filtered, giving the title compound (6.1 g) as a light brown solid. MS (ES)⁺: 452/454 (M+Na)⁺

### Intermediate 42—tert-butyl (3-((6-mercapto-1-oxophthalazin-2(1H)-yl)methyl)phenyl)carbamate

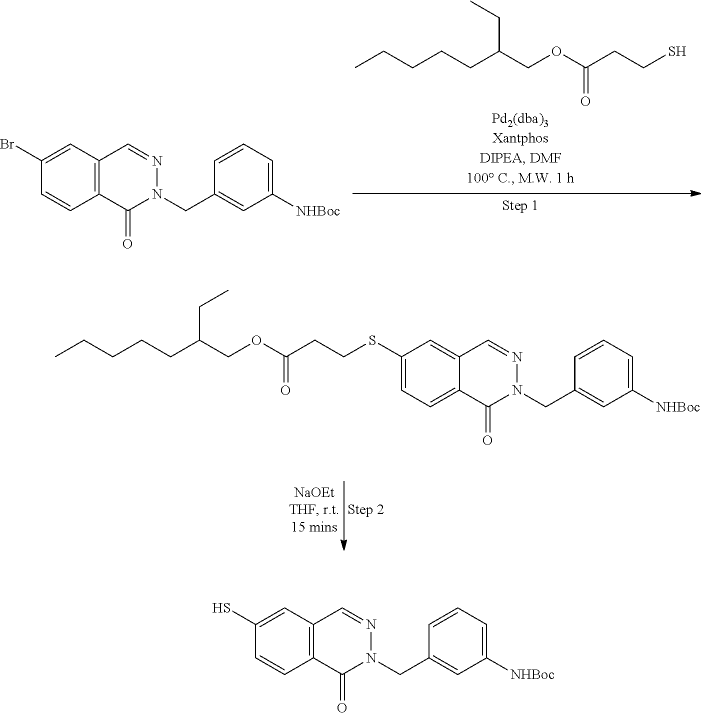 [SMILES: CCCCCC(CC)COC(=O)CCS ; CC(C)(C)OC(=O)Nc1cccc(Cn2ncc3cc(Br)ccc3c2=O)c1 ; CCCCCC(CC)COC(=O)CCSc1ccc2c(=O)n(Cc3cccc(NC(=O)OC(C)(C)C)c3)ncc2c1 ; CC(C)(C)OC(=O)Nc1cccc(Cn2ncc3cc(S)ccc3c2=O)c1]

A degassed mixture of tert-butyl (3-((6-bromo-1-oxophthalazin-2(1H)-yl)methyl)phenyl)carbamate (Intermediate 41, 1.000 g, 1 Eq, 2.324 mmol), 2-ethylhexyl 3-mercaptopropanoate (532.8 mg, 1.05 Eq, 2.440 mmol), DIPEA (600.7 mg, 810 μL, 2 Eq, 4.648 mmol), xantphos (134.5 mg, 0.1 Eq, 232.4 μmol), and Pd₂(dba)₃ (106.4 mg, 0.05 Eq, 116.2 μmol) in 1,4-dioxane (7.0 mL) was heated to 100° C. under N₂ for 2 h. After cooling to RT, the reaction mixture was diluted with water (10 mL) and extracted with EtOAc (10×5 mL). The organic phase was collected, dried (MgSO₄) and evaporated under reduced pressure. The crude product was purified by chromatography on silica gel to afford 2-ethylhexyl 3-((2-(3-((tert-butoxycarbonyl)amino)benzyl)-1-oxo-1,2-dihydrophthalazin-6-yl)thio)propanoate (0.925 g) as an orange oil. MS (ES⁺): 590.8 (M+Na)⁺

### Step 2

Sodium ethoxide (2.360 g, 2.72 mL, 21% Wt, 2 Eq, 7.281 mmol) was added to a stirred solution of 2-ethylhexyl 3-((2-(3-((tert-butoxycarbonyl)amino)benzyl)-1-oxo-1,2-dihydrophthalazin-6-yl)thio)propanoate (2.067 g, 1 Eq, 3.641 mmol) in THF (12 mL) at 0° C. and the resulting solution was stirred for 1 h while warming up to RT. The reaction was diluted with DCM (20 mL) and quenched with sat. aq. NH₄Cl. (10 mL) The organic phase was separated, and the aqueous phase was extracted with EtOAc (2×20 mL). The combined organic phases were dried over MgSO₄, filtered, and the volatiles were removed under reduced pressure to afford the title compound (0.877 g). MS (ES⁺): 406 (M+Na)⁺

### Intermediate 43—1-methyl-1H-pyrazole-3-thiol

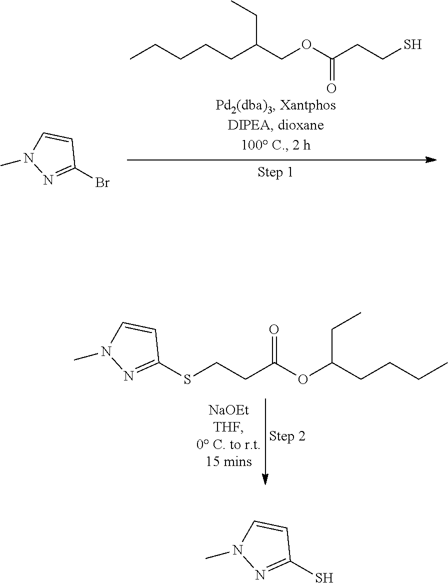

### Step 1

A degassed mixture of 3-bromo-1-methyl-1H-pyrazole (0.500 g, 315 μL, 1 Eq, 3.11 mmol), 2-ethylhexyl 3-mercaptopropanoate (746 mg, 1.1 Eq, 3.42 mmol), DIPEA (803 mg, 1.08 mL, 2 Eq, 6.21 mmol), xantphos (180 mg, 0.1 Eq, 311 μmol), and Pd₂(dba)₃ (142 mg, 0.05 Eq, 155 μmol) in 1,4-dioxane (10 mL) was heated to 100° C. for 20 h. After cooling to RT the reaction mixture was quenched with water (10 mL) and extracted with EtOAc (3×10 mL). The organic phase was collected, dried (MgSO₄) and evaporated under reduced pressure. The crude product was purified by chromatography on silica gel to afford 2-ethylhexyl 3-((1-methyl-1H-pyrazol-3-yl)thio)propanoate (0.803 g) as an orange oil. MS (ES⁺): 299 (M+H)⁺

### Step 2

Sodium ethoxide (1.74 g, 2.01 mL, 21% Wt, 2 Eq, 5.38 mmol) was added to a stirred solution of 2-ethylhexyl 3-((1-methyl-1H-pyrazol-3-yl)thio)propanoate (0.803 g, 1 Eq, 2.69 mmol) in THF (9.0 mL) at 0° C. and the resulting solution was stirred for 1 h while warming up to RT. The reaction was diluted with DCM (10 mL) and quenched with sat. aq. NH₄Cl. (5 mL) The organic phase was separated and the aqueous phase was extracted with EtOAc (2×10 mL). The combined organic phases were dried over MgSO₄, filtered, and the volatiles were removed under reduced pressure. The aqueous layer was concentrated, diluted in MeOH, and adsorbed on silica-gel. Purification by chromatography on silica gel afforded the title compound (0.125 g) as a pale orange oil. ¹H NMR (400 MHz, DMSO-d6) δ 7.63 (d, J=2.2 Hz, 1H), 6.12 (d, J=2.3 Hz, 1H), 3.74 (s, 3H). (—SH signal could not be observed).

### Intermediate 44—benzo[d][1,3]dioxole-5-thiol

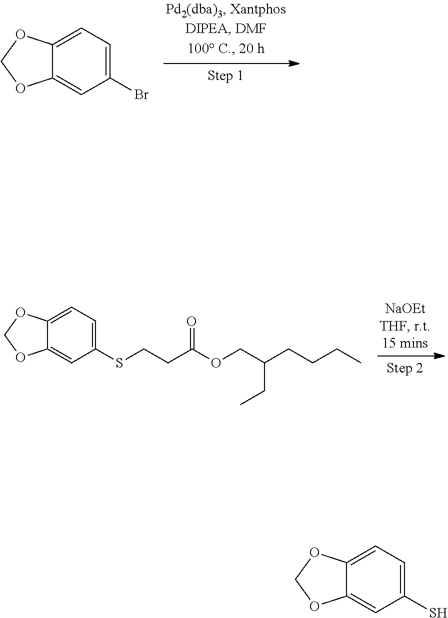 [SMILES: Brc1ccc2c(c1)OCO2 ; CCCCC(CC)COC(=O)CCSc1ccc2c(c1)OCO2 ; Sc1ccc2c(c1)OCO2]

### Step 1

A degassed mixture of 5-bromobenzo[d][1,3]dioxole (0.850 g, 1 Eq, 4.23 mmol), 2-ethylhexyl 3-mercaptopropanoate (1.02 g, 1.1 Eq, 4.65 mmol), Xantphos (245 mg, 0.1 Eq, 423 μmol), Pd₂(dba)₃ (194 mg, 0.05 Eq, 211 μmol), and DIPEA (1.09 g, 1.47 mL, 2 Eq, 8.46 mmol) in 1,4-Dioxane (13 mL) was heated to 100° C. for 20 h. After cooling to RT, the reaction mixture was filtered through a celite pad and evaporated to dryness. The crude product was purified by chromatography on silica gel to afford 2-ethylhexyl 3-(benzo[d][1,3]dioxol-5-ylthio)propanoate (1.478 g) as an orange oil. MS (ES⁺): 339 (M+H)⁺

### Step 2

Sodium ethoxide (3.092 g, 3.56 mL, 21% Wt, 2.2 Eq, 9.542 mmol) was added to a stirred solution of 2-ethylhexyl 3-(benzo[d][1,3]dioxol-5-ylthio)propanoate (1.468 g, 1 Eq, 4.337 mmol) in THF (15 mL) at 0° C. and the resulting solution was stirred for 1 h while warming up to RT. The reaction mixture was filtered over a pad of silica before being evaporated to dryness. The crude product was purified by chromatography on silica gel to afford the title compound (0.379 g) as an orange oil. ¹H NMR (400 MHz, DMSO-d6) δ 6.90 (d, J=1.9 Hz, 1H), 6.82 (d, J=8.0 Hz, 1H), 6.76 (dd, J=8.1, 1.8 Hz, 1H), 5.98 (s, 2H). (—SH signal could not be observed). The product was analysed by LCMS (Agilent, Cortecs C18+, 90 Å, 2.7 μm, 2.1 mm×30 mm, Acidic (0.1% Formic acid), 3 min method, 5-100% MeCN/water): 3056-364 F9-16 (mislabelled), m/z 481 (M+H)⁺ (ES⁺); at 1.84 min, 98% purity at 254 nm.

### Intermediate 45—2-chloro-3-(chloromethyl)pyridine

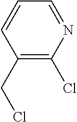

To a solution of (2-chloropyridin-3-yl)methanol (1 g, 7 mmol) in DCM (10 mL) was drop-wised SOCl₂ (4.17 g, 35 mmol) at 0° C., and the reaction mixture was stirred at RT for 3 h. After LCMS indicated the reaction was complete, the mixture was concentrated at 30° C. under reduced pressure to remove DCM and the residual SOCl₂. The mixture was quenched with ice water (10 mL), adjusted to pH=9 by adding aq. 2 N K₂CO₃ solution, and extracted with DCM (10 mL×2). The organic layer was washed with brine, dried over Na₂SO₄, filtered and concentrated at 30° C. under reduced pressure to give the title compound (1 g) as a yellow oil. MS (ES⁺): 162.2 (M+H)⁺

### Intermediate 46—2-((2-chloropyridin-3-yl)methyl)-6-mercaptophthalazin-1(2H)-one

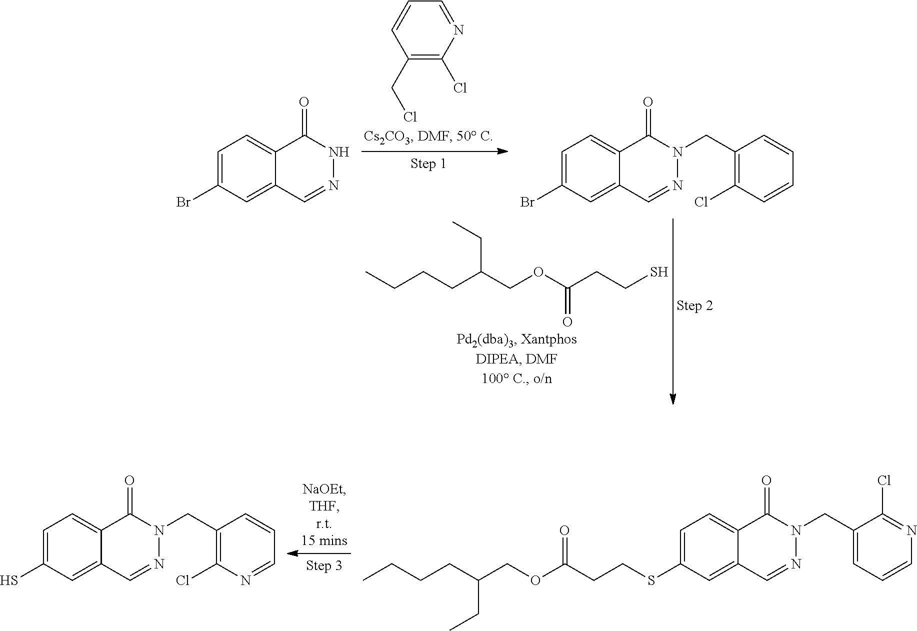 [SMILES: O=c1[nH]ncc2cc(Br)ccc12 ; ClCc1cccnc1Cl ; O=c1c2ccc(Br)cc2cnn1Cc1ccccc1Cl ; CCCCC(CC)COC(=O)CCS ; O=c1c2ccc(S)cc2cnn1Cc1cccnc1Cl ; CCCCC(CC)COC(=O)CCSc1ccc2c(=O)n(Cc3cccnc3Cl)ncc2c1]

### Step 1

To the solution of 6-bromophthalazin-1(2H)-one (1.34 g, 6 mmol) and K₂CO₃ (1.66 g, 12 mmol) in DMF (10 mL) was added 2-chloro-3-(chloromethyl)pyridine (Intermediate 45, 1 g, 6 mmol) at RT, and the reaction mixture was stirred at 90° C. overnight. LCMS analysis indicated the reaction to be complete and the reaction mixture was diluted with water (20 mL) and filtered. The filtered solid was dissolved in DCM (20 mL), washed by water and brine, dried over Na₂SO₄, filtered and concentrated at 30° C. under reduced pressure. The residual solid was triturated with a n-hexane and MTBE (10/1) mixed solvent; filtered and dried at 30° C. under vacuum to give 6-bromo-2-((2-chloropyridin-3-yl)methyl)phthalazin-1(2H)-one (1.2 g) as a yellow solid. MS (ES⁺): 350.0 (M+H)⁺

### Step 2

To the solution of 6-bromo-2-((2-chloropyridin-3-yl)methyl)phthalazin-1(2H)-one (1.2 g, 3.4 mmol), Pd₂(dba)₃ (311 mg, 0.34 mmol), Xantphos (393 mg, 0.68 mmol), DIPEA (1.3 g, 10.2 mmol) in DMF (20 mL) was added 2-ethylhexyl 3-mercaptopropanoate (1.1 g, 5 mmol) at RT under N₂, and the reaction mixture was stirred at 100° C. overnight. After LCMS indicated the reaction was complete, the reaction mixture was filtered through Celite and washed with EtOAc. The filtrate was diluted with water (30 mL) and extracted with EtOAc (20 mL×3). The combined organic layer was washed by brine, dried over Na₂SO₄, filtered and concentrated at 45° C. under reduced pressure. The residue was purified by flash column chromatography to give 2-ethylhexyl 3-(2-((2-chloropyridin-3-yl)methyl)-1-oxo-1,2-dihydrophthalazin-6-ylthio)propanoate (1.5 g) as a yellow solid. MS (ES⁺): 488.0 (M+H)⁺

### Step 3

To a solution of 2-ethylhexyl 3-(2-((2-chloropyridin-3-yl)methyl)-1-oxo-1,2-dihydrophthalazin-6-ylthio)propanoate (1.5 g, 3 mmol) in THF (20 mL) was added NaOEt (1.6 mL, 2.5N in EtOH, 4 mmol) at RT, and the reaction mixture was stirred at room temperature for 15 min. After LCMS indicated the reaction was complete, the reaction mixture was quenched and adjusted pH=5 by drop-wising 0.5 N HCl aq., and extracted with EtOAc (20 mL×2). The combined organic layer was washed with brine, dried over Na₂SO₄, filtered and concentrated at 40° C. under reduced pressure to give the title compound (900 mg) as brown solid. MS (ES⁺): 304.0 (M+H)⁺

### Intermediate 47—3-(chloromethyl)-1-methyl-1H-pyrazole

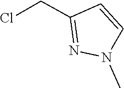

To the solution of (1-methyl-1H-pyrazol-3-yl)methanol (3 g, 27 mmol) in DCM (30 mL) was drop-wised SOCl₂ (16 g, 135 mmol) at 0° C., and the reaction mixture was stirred at RT for 3 h. After LCMS indicated the reaction was complete, the mixture was concentrated at 30° C. under reduced pressure to remove DCM and most of the SOCl₂. The residue was quenched with ice water (30 mL), adjusted to pH=9 by addition of 2 N K₂CO₃ aqueous solution, and extracted with DCM (30 mL×2). The organic layer was washed with brine, dried over Na₂SO₄, filtered and concentrated at 30° C. under reduced pressure to give (3 g) as a yellow oil. MS (ES⁺): 131.2 (M+H)⁺

### Intermediate 48—6-mercapto-2-((1-methyl-1H-pyrazol-3-yl)methyl)phthalazin-1(2H)-one

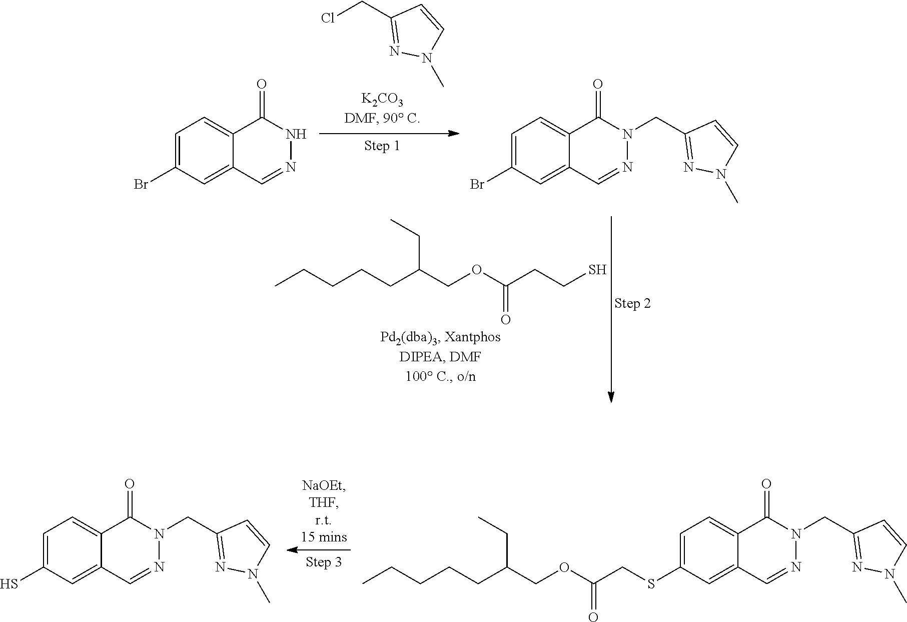

### Step 1

To the solution of 6-bromophthalazin-1(2H)-one (3.5 g, 15.6 mmol) and K₂CO₃ (4.14 g, 30 mmol) in DMF (30 mL) was added 3-(chloromethyl)-1-methyl-1H-pyrazole (Intermediate 47, 2.1 g, 16 mmol) at RT, and the reaction mixture was stirred at 90° C. overnight. After LCMS indicated the reaction was complete, the reaction mixture was filtered through Celite and washed with EtOAc. The filtrate was diluted with water (30 mL) and extracted with EtOAc (20 mL×3). The combined organic layer was washed by brine, dried over Na₂SO₄, filtered and concentrated at 45° C. under reduced pressure. The residue was purified by flash column chromatography to give 6-bromo-2-((1-methyl-1H-pyrazol-3-yl)methyl)phthalazin-1(2H)-one (3.1 g) as a yellow solid. MS (ES⁺): 319.0 (M+H)⁺

### Step 2

To the solution of 6-bromo-2-((1-methyl-1H-pyrazol-3-yl)methyl)phthalazin-1(2H)-one (3.1 g, 9.7 mmol), Pd₂(dba)₃ (888 mg, 0.97 mmol), Xantphos (1.12 g, 1.94 mmol), DIPEA (3.7 g, 29 mmol) in DMF (20 mL) was added 2-ethylhexyl 3-mercaptopropanoate (2.3 g, 9.7 mmol) at RT under N₂, and the reaction mixture was stirred at 100° C. overnight. After LCMS indicated the reaction was complete, the reaction mixture was filtered through Celite and washed with EtOAc. The filtrate was diluted with water (30 mL) and extracted with EtOAc (20 mL×3). The combined organic layer was washed by brine, dried over Na₂SO₄, filtered and concentrated at 45° C. under reduced pressure. The residue was purified by flash column chromatography to give 2-ethylheptyl 2-(2-((1-methyl-1H-pyrazol-3-yl)methyl)-1-oxo-1,2-dihydrophthalazin-6-ylthio)acetate (4 g) as a yellow solid. MS (ES⁺): 457.0 (M+H)⁺

### Step 3

To a solution of 2-ethylheptyl 2-(2-((1-methyl-1H-pyrazol-3-yl)methyl)-1-oxo-1,2-dihydrophthalazin-6-ylthio)acetate (4 g, 8.7 mmol) in THF (40 mL) was added NaOEt (3.52 mL, 2.5N in EtOH, 8.8 mmol) at RT, and the reaction mixture was stirred at RT for 15 min. After LCMS indicated the reaction was complete, the reaction mixture was quenched and adjusted pH=5 by drop-wising 0.5 N HCl aq., and extracted with EtOAc (20 mL×2). The combined organic layer was washed with brine, dried over Na₂SO₄, filtered and concentrated at 40° C. under reduced pressure to give the title compound (2.1 g) as brown solid. MS (ES⁺): 273.0 (M+H)⁻

### Intermediate 49—6-mercapto-2-((1-(tetrahydro-2H-pyran-2-yl)-1H-pyrazol-3-yl)methyl)phthalazin-1(2H)-one

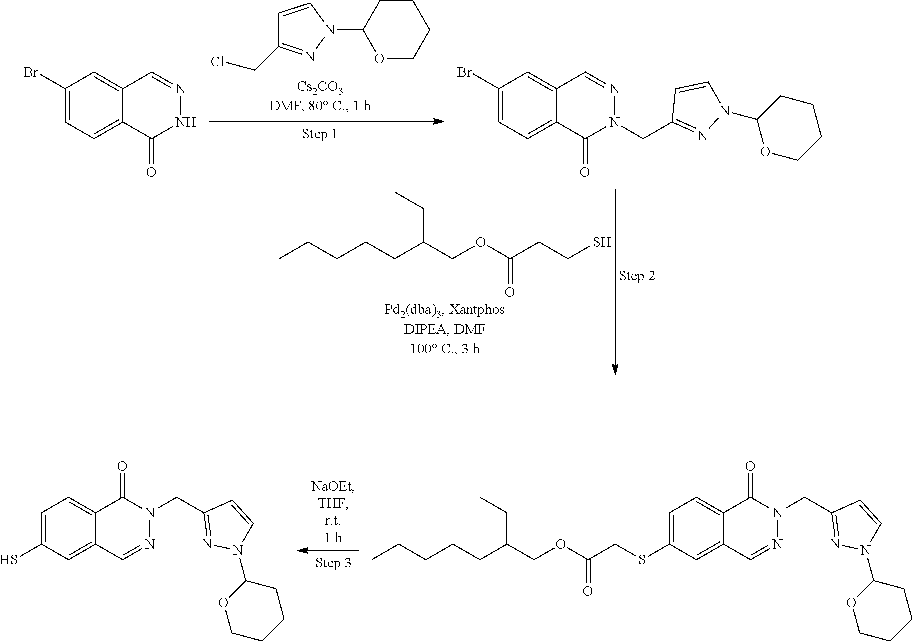 [SMILES: ClCc1ccn(C2CCCCO2)n1 ; O=c1[nH]ncc2cc(Br)ccc12 ; O=c1c2ccc(Br)cc2cnn1Cc1ccn(C2CCCCO2)n1 ; CCCCCC(CC)COC(=O)CCS ; O=c1c2ccc(S)cc2cnn1Cc1ccn(C2CCCCO2)n1 ; CCCCCC(CC)COC(=O)CSc1ccc2c(=O)n(Cc3ccn(C4CCCCO4)n3)ncc2c1]

### Step 1

A suspension of 6-bromophthalazin-1(2H)-one (2.117 g, 98% Wt, 1 Eq, 9.217 mmol) and cesium carbonate (6.006 g, 2.0 Eq, 18.43 mmol) in DMF (40 mL) was stirred at 80° C. for 1 h and then allowed to cool to RT. A solution of 3-(chloromethyl)-1-(tetrahydro-2H-pyran-2-yl)-1H-pyrazole (2.055 g, 9.217 mmol) in DMF (10 mL) was added and the reaction mixture was stirred at RT for 20 h. EtOAc (150 mL) was added and the reaction mixture was washed with water (100 mL). The organic layer was collected and the aqueous was extracted with EtOAc (3×100 mL). The combined organic extracts were washed with 50% brine (2×100 mL), brine (100 mL) and concentrated in vacuo to afford the crude product. The crude product was purified by chromatography on silica gel to afford impure product. The crude product was purified by chromatography on silica gel to afford 6-bromo-2-((1-(tetrahydro-2H-pyran-2-yl)-1H-pyrazol-3-yl)methyl)phthalazin-1(2H)-one (Intermediate 49A, 2.192 g) as an off-white solid. MS (ES⁺): 389/391 (M+H)⁺

### Step 2

A solution of 6-bromo-2-((1-(tetrahydro-2H-pyran-2-yl)-1H-pyrazol-3-yl)methyl)phthalazin-1(2H)-one (1.47 g, 3.40 mmol) in DMF (30 mL) was sparged with N₂ for 5 minutes. DIPEA (879 mg, 1.18 mL, 2.00 Eq, 6.80 mmol), Xantphos (197 mg, 340 μmol), Pd₂(dba)₃ (156 mg, 170 μmol) and 2-ethylhexyl 3-mercaptopropanoate (852 mg, 3.90 mmol) were added sequentially and the reaction mixture was stirred at 100° C. for 90 min. 2-ethylhexyl 3-mercaptopropanoate (0.20 mL, 0.876 mmol) was added and stirred at 100° C. for 90 min. The reaction mixture was concentrated in vacuo and the residue was azeotroped with toluene (3 times) to afford the crude product. The crude product was purified by chromatography on silica gel to afford 2-ethylhexyl 3-((1-oxo-2-((1-(tetrahydro-2H-pyran-2-yl)-1H-pyrazol-3-yl)methyl)-1,2-dihydrophthalazin-6-yl)thio)propanoate (1.648 g) as a thick yellow oil. MS (ES⁺): 527 (M+H)⁺

### Step 3

A stirred solution of 2-ethylhexyl 3-((1-oxo-2-((1-(tetrahydro-2H-pyran-2-yl)-1H-pyrazol-3-yl)methyl)-1,2-dihydrophthalazin-6-yl)thio)propanoate (1.64 g, 2.77 mmol) in THF (25 mL) was treated with sodium ethoxide (2.13 g, 6.56 mmol) dropwise. The reaction mixture was stirred at room temperature for 1 h and then diluted with DCM (100 mL) and sat. aq. NH₄Cl (100 mL). The organic layer was collected and the aqueous was extracted with DCM (2×50 mL). The combined organic extracts were washed with 50% brine (50 mL), dried (phase separator) and concentrated in vacuo to afford the crude product. The crude product was purified by chromatography on silica gel to afford the title compound (715 mg) as a pale pink solid. ¹H NMR (400 MHz, DMSO-d6) δ 8.29 (s, 1H), 8.10 (d, J=8.4 Hz, 1H), 7.83-7.72 (m, 3H), 6.34 (s, 1H), 6.15 (d, J=2.4 Hz, 1H), 5.31 (dd, J=10.3, 2.4 Hz, 1H), 5.24 (s, 2H), 3.94-3.85 (m, 1H), 3.58 (ddd, J=11.5, 8.6, 6.3 Hz, 1H), 2.03 (tdd, J=12.5, 10.0, 3.6 Hz, 1H), 1.95-1.81 (m, 2H), 1.70-1.57 (m, 1H), 1.50 (tq, J=8.0, 3.9 Hz, 2H). MS (ES⁺): 343 (M+H)⁺

### Intermediate 50—2-((1-cyclopropyl-1H-pyrazol-3-yl)methyl)-6-mercaptophthalazin-1(2H)-one

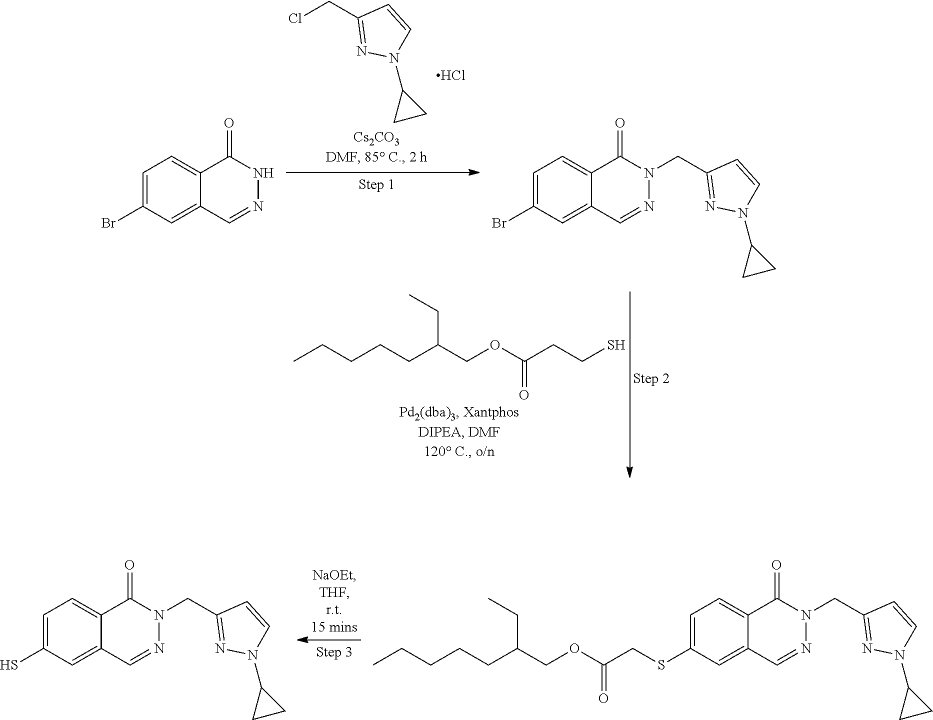

### Step 1

A stirred suspension of 6-bromophthalazin-1(2H)-one (407 mg, 1 Eq, 1.81 mmol) and cesium carbonate (2.36 g, 4.0 Eq, 7.24 mmol) in DMF (10 mL) was heated to 85° C. for 2 h and then allowed to cool to RT. A solution of 3-(chloromethyl)-1-cyclopropyl-1H-pyrazole hydrochloride (Intermediate 6, 349 mg, 100% Wt, 1 Eq, 1.81 mmol) in DMF (2 mL) was added and the reaction mixture was stirred at RT for 18 h. Water (50 mL) was added and the resulting solid was collected by filtration, washing with water to afford 6-bromo-2-((1-cyclopropyl-1H-pyrazol-3-yl)methyl)phthalazin-1(2H)-one (500 mg) as a yellow solid. MS (ES⁺): 345 (M+H)⁺

### Step 2

A suspension of 6-bromo-2-((1-cyclopropyl-1H-pyrazol-3-yl)methyl)phthalazin-1(2H)-one (500 mg, 79% Wt, 1 Eq, 1.14 mmol) in DMF (8 mL) was sparged with N₂ for 10 minutes. DIPEA (223 mg, 300 μL, 1.51 Eq, 1.72 mmol), Xantphos (84 mg, 0.13 Eq, 0.15 mmol), Pd₂(dba)₃ (66 mg, 0.063 Eq, 72 μmol) and 2-ethylhexyl 3-mercaptopropanoate (384 mg, 400 μL, 1.54 Eq, 1.76 mmol) were added sequentially and the reaction mixture was stirred at 120° C. under N₂ for 1 h. The reaction mixture was allowed to cool to RT and then partitioned between EtOAc (50 mL) and sat. aq. NaHCO₃ (50 mL). The organic layer was collected and the aqueous was extracted with EtOAc (50 mL). The combined organic extracts were washed with 50% brine (50 mL), brine (50 mL), dried (MgSO₄) and concentrated in vacuo to afford the crude product. The crude product was purified by chromatography on silica gel to afford 2-ethylhexyl 3-((2-((1-cyclopropyl-1H-pyrazol-3-yl)methyl)-1-oxo-1,2-dihydrophthalazin-6-yl)thio)propanoate (630 mg) as a yellow oil. MS (ES⁺): 483 (M+H)⁺

### Step 3

A solution of 2-ethylhexyl 3-((2-((1-cyclopropyl-1H-pyrazol-3-yl)methyl)-1-oxo-1,2-dihydrophthalazin-6-yl)thio)propanoate (630 mg, 88% Wt, 1 Eq, 1.15 mmol) in THF (10 mL) was treated with sodium ethoxide (1.04 g, 1.20 mL, 21% Wt, 2.80 Eq, 3.21 mmol). The reaction mixture was stirred at RT for 15 minutes and then water (10 mL) was added. The mixture was acidified with 1 M HCl (aq.) and then extracted with EtOAc (3×40 mL). The combined organic extracts were washed with brine (40 mL), dried (MgSO₄) and concentrated in vacuo to afford the crude product as an orange solid. The crude product was purified by chromatography on silica gel to afford the title compound (379 mg) as a pale yellow solid. MS (ES⁺): 299 (M+H)⁺

### Intermediate 51—6-bromo-2-((4-methyl-1-((2-(trimethylsilyl)ethoxy)methyl)-1H-pyrazol-3-yl)methyl)phthalazin-1(2H)-one

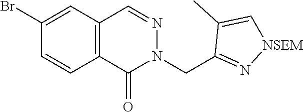 [SMILES: Cc1cn(COCC[Si](C)(C)C)nc1Cn1ncc2cc(Br)ccc2c1=O]

A stirred suspension of 6-bromophthalazin-1(2H)-one (120 mg, 1.55 Eq, 533 μmol) and cesium carbonate (224 mg, 2.0 Eq, 687 μmol) in DMF (2 mL) was heated to 85° C. for 90 min. and then allowed to cool to RT. A solution of 3-(chloromethyl)-4-methyl-1-((2-(trimethylsilyl)ethoxy)methyl)-1H-pyrazole (Intermediate 10, 121 mg, 74% Wt, 1 Eq, 343 μmol) in DMF (2 mL) was added and the reaction mixture was stirred at RT for 18 h. The reaction mixture was diluted with DCM (5 mL) and washed with sat. aq. NaHCO₃ (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford the title compound (120 mg) as a clear yellow oil that solidified on standing. MS (ES⁺): 449/451 (M+H)⁺

### Intermediate 52—6-bromo-2-((5-methyl-1H-pyrazol-3-yl)methyl)phthalazin-1(2H)-one

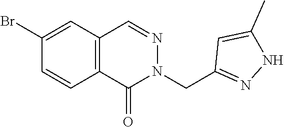 [SMILES: Cc1cc(Cn2ncc3cc(Br)ccc3c2=O)n[nH]1]

A stirred suspension of 6-bromophthalazin-1(2H)-one (865 mg, 1.74 Eq, 3.84 mmol) and cesium carbonate (2.50 g, 3.48 Eq, 7.67 mmol) in DMF (25 mL) was heated to 85° C. for 90 min. and then allowed to cool to RT. 3-(chloromethyl)-5-methyl-1H-pyrazole, HCl (368 mg, 1 Eq, 2.20 mmol) was added portionwise and the reaction mixture was stirred at RT for 18 h. Further cesium carbonate (2.50 g, 3.48 Eq, 7.67 mmol) was added and the mixture heated to 50° C. for 4 h, then allowed to cool to RT. The reaction mixture was diluted with EtOAc (100 mL) and washed with sat. aq. NaHCO₃ (100 mL). The organic layer was collected and the aqueous was extracted with EtOAc (2×50 mL). The combined organic extracts were washed with brine (100 mL), dried (MgSO₄) and concentrated in vacuo to afford the crude product. The crude product was purified by chromatography on silica gel to afford a mixture of the title compound (370 mg, 0.58 mmol) and starting material (370 mg) as a pale yellow solid. MS (ES⁺): 319/321 (M+H)⁺

### Intermediate 53—6-mercaptophthalazin-1(2H)-one

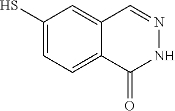 [SMILES: O=c1[nH]ncc2cc(S)ccc12]

A mixture of 6-bromophthalazin-1(2H)-one (0.60 g, 1 Eq, 2.7 mmol), 2-ethylhexyl 3-mercaptopropanoate (0.61 g, 0.64 mL, 1.05 Eq, 2.8 mmol), cesium carbonate (1.7 g, 2 Eq, 5.3 mmol), Xantphos (0.15 g, 0.1 Eq, 0.27 mmol), and Pd₂(dba)₃ (0.12 g, 0.05 Eq, 0.13 mmol) in DMF (12 mL) was heated to 100° C. for 12 h. After cooling to RT the reaction mixture was diluted with water (50 mL). then extracted with DCM (20 mL). The aqueous layer was acidified with 1 M HCl, then extracted with EtOAc (2×50 mL). The organic phase was collected, dried (MgSO₄) and evaporated under reduced pressure, giving the title compound (0.33 g) as a sticky orange solid. MS (ES⁺): 179 (M+H)⁺

### Intermediate 54—3-iodo-1-(tetrahydro-2H-pyran-2-yl)-1H-pyrazole

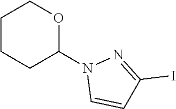 [SMILES: Ic1ccn(C2CCCCO2)n1]

To the solution of 3-iodo-1H-pyrazole (4 g, 21 mmol) and 3,4-dihydro-2H-pyran (1.99 g, 23.1 mmol) in toluene (100 mL) was added TsOH (361.2 mg, 2.1 mmol) at RT, and the reaction mixture was stirred at 120° C. overnight. After LCMS indicated the reaction is completed, the reaction mixture was filtered through Celite and washed with EtOAc. The filtrate was diluted with water (50 mL) and extracted with EtOAc (40 mL×3). The combined organic layer was washed by brine, dried over Na₂SO₄, filtered and concentrated at 50° C. under reduced pressure. The residue was purified by flash column chromatography, giving the title compound (4.4 g) as a yellow solid. MS (ES⁺): 278.9 (M+H)⁺

### Intermediate 55—6-mercapto-2-((6-methylpyridin-2-yl)methyl)phthalazin-1(2H)-one

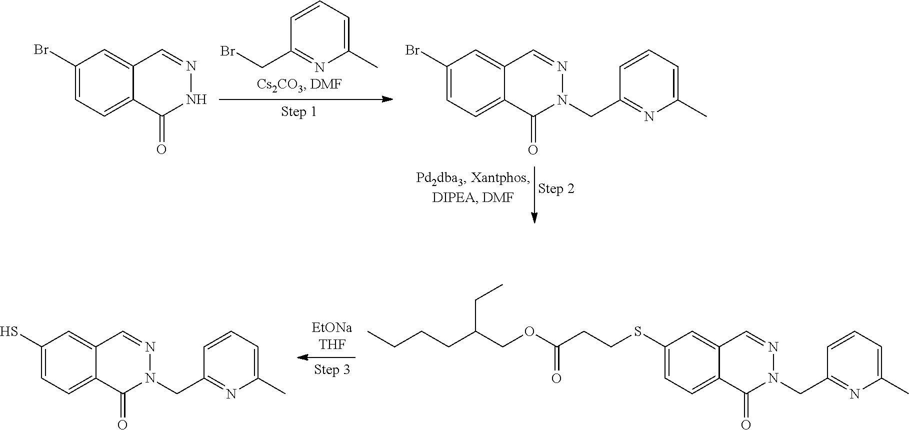

### Step 1

A mixture of 6-bromophthalazin-1(2H)-one (2.00 g, 1 Eq, 8.89 mmol) and cesium carbonate (5.79 g, 2 Eq, 17.8 mmol) in DMF (45 mL) was stirred at 70° C. for 45 min. 2-(bromomethyl)-6-methylpyridine (1.74 g, 1.05 Eq, 9.33 mmol) was added and the mixture stirred at RT overnight, then poured into ice/water. The resulting precipitate was filtered, washed with water then dried in a vacuum oven at 40° C. overnight to afford 6-bromo-2-((6-methylpyridin-2-yl)methyl)phthalazin-1(2H)-one (2.43 g, 7.3 mmol). MS (ES⁺) 330/332 (M+H)⁺

### Step 2

A solution of 6-bromo-2-((6-methylpyridin-2-yl)methyl)phthalazin-1(2H)-one (1.424 g, 99% Wt, 1 Eq, 4.270 mmol) in DMF (38.0 mL) was sparged with N₂ for 5 minutes. DIPEA (1.104 g, 1.49 mL, 2.00 Eq, 8.539 mmol), Xantphos (247.0 mg, 0.10 Eq, 427.0 μmol), Pd₂(dba)₃ (195.5 mg, 0.05 Eq, 213.5 μmol) and 2-ethylhexyl 3-mercaptopropanoate (1.119 g, 1.17 mL, 1.20 Eq, 5.123 mmol) were added sequentially and the reaction mixture was stirred at 100° C. for 2 h. The reaction was cooled to RT, concentrated under vacuum and azeotroped with PhMe. The residue was partitioned between EtOAc (50 mL) and water (50 mL). The aqueous was extracted with EtOAc (70 mL×3) and the combined organics were washed with a 1:1 mixture of brine and water (100 mL×3), dried (MgSO₄), filtered and concentrated under reduced pressure to give a crude dark orange oil (2.92 g). The crude was dissolved in DCM (50 mL), concentrated onto silica and purified by chromatography to afford 2-ethylhexyl 3-((2-((6-methylpyridin-2-yl)methyl)-1-oxo-1,2-dihydrophthalazin-6-yl)thio)propanoate (2.02 g, 4.2 mmol) as a thick orange oil. ¹H NMR (400 MHz, DMSO) δ 8.39 (s, 1H), 8.14 (d, J=8.5 Hz, 1H), 7.87 (d, J=1.9 Hz, 1H), 7.74 (dd, J=8.5, 1.9 Hz, 1H), 7.60 (t, J=7.7 Hz, 1H), 7.13 (d, J=7.7 Hz, 1H), 6.90 (d, J=7.7 Hz, 1H), 5.36 (s, 2H), 3.96 (d, J=5.8 Hz, 2H), 3.37 (t, J=6.8 Hz, 2H), 2.76 (t, J=6.8 Hz, 2H), 2.42 (s, 3H), 1.55-1.47 (m, 1H), 1.34-1.23 (m, 2H), 1.24-1.20 (m, 6H), 0.87-0.79 (m, 6H). MS (ES)⁺: 468 (M+H)⁺

### Step 3

A solution of 2-ethylhexyl 3-((2-((6-methylpyridin-2-yl)methyl)-1-oxo-1,2-dihydrophthalazin-6-yl)thio)propanoate (2.02 g, 1 Eq, 4.32 mmol) in THF (50.0 mL) at 0° C. was treated sodium ethoxide (3.50 g, 4.03 mL, 21% Wt, 2.5 Eq, 10.8 mmol). The reaction mixture was stirred for 1.5 h at 0° C. before adding water (50 mL). The mixture was acidified with 1 M HCl (aq.) (˜6-8 mL; reaching pH 4) then warmed to RT and extracted with EtOAc (3×40 mL). The combined organics were washed with brine (60 mL×2) then water (60 mL). The resulting suspension was concentrated under reduced pressure to give a light brown solid (3.79 g). The crude was dissolved in DCM (20 mL), concentrated onto silica and purified by chromatography to afford the title compound (894.2 mg, 3.1 mmol) as a pale orange solid. ¹H NMR (400 MHz, DMSO-d6) δ 8.33 (s, 1H), 8.10 (d, J=8.4 Hz, 1H), 7.84 (d, J=1.9 Hz, 1H), 7.77 (dd, J=8.3, 1.9 Hz, 1H), 7.60 (t, J=7.7 Hz, 1H), 7.13 (d, J=7.6 Hz, 1H), 6.90 (d, J=7.7 Hz, 1H), 6.35 (s, 1H), 5.34 (s, 2H), 2.42 (s, 3H). MS (ES)⁺: 284 (M+H)⁺

### Intermediates 56 and 57—5-fluoro-6-(phenylthio)phthalazin-1(2H)-one and 7-fluoro-6-(phenylthio)phthalazin-1(2H)-one

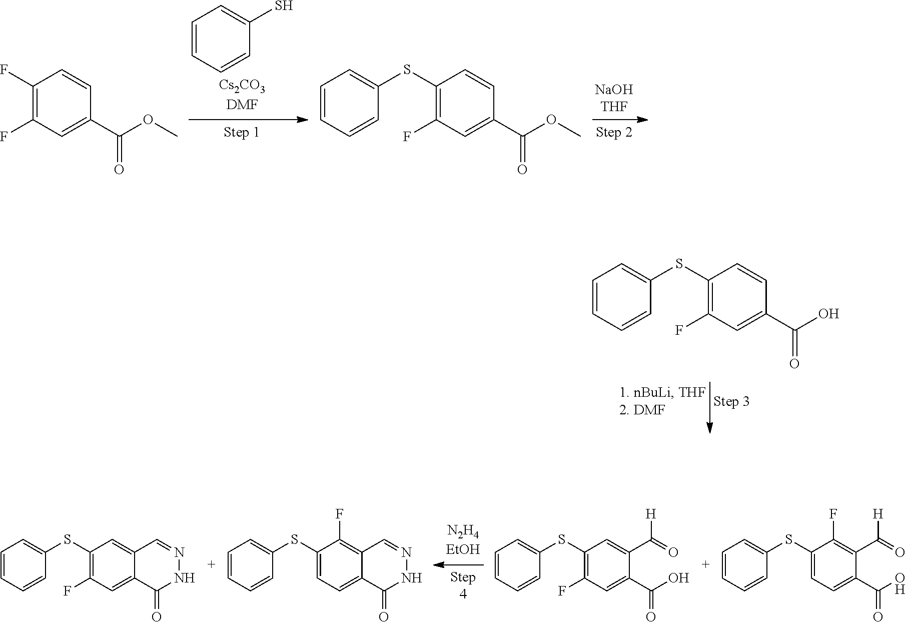

### Step 1

A stirred suspension of methyl 2-bromo-4,5-difluorobenzoate (2.019 g, 99% Wt, 1 Eq, 7.962 mmol) and cesium carbonate (3.144 g, 1.212 Eq, 9.649 mmol) in DMF (20 mL) was treated with benzenethiol (885.2 mg, 825.0 μL, 1.009 Eq, 8.035 mmol) dropwise. The reaction mixture was stirred at RT for 3 h and then diluted with EtOAc (50 mL) and washed with water (50 mL). The organic layer was collected and the aqueous was extracted with EtOAc (3×25 mL). The combined organic extracts were washed with 50% brine (50 mL), brine (50 mL), dried (MgSO₄) and concentrated in vacuo to afford the crude product as a pale yellow oil. The crude product was purified by chromatography to afford methyl 2-bromo-5-fluoro-4-(phenylthio)benzoate (2.64 g) as a colourless oil. MS (ES⁺): 341/343 (M+H)⁺

### Step 2

A solution of methyl 2-bromo-5-fluoro-4-(phenylthio)benzoate (2.209 g, 99% Wt, 1 Eq, 6.410 mmol) and NaOH (0.54 g, 6.730 mL, 2.00 molar, 2.1 Eq, 13.46 mmol) in THF (20 mL) was stirred at RT for 18 h. The mixture was acidified with 1 M HCl (aq.) (˜20 mL) and then extracted with DCM (2×50 mL). The combined organic extracts were dried (phase separator) and concentrated in vacuo to afford 2-bromo-5-fluoro-4-(phenylthio)benzoic acid (2.136 g) as a white solid. MS (ES⁺): 327/329 (M+H)⁺

### Step 3

n-Butyllithium (1.9 M in hexanes) (864 mg, 7.10 mL, 1.90 molar, 2.13 Eq, 13.5 mmol) was added to THF (7 mL) under N₂ at −78° C. A solution of 2-bromo-5-fluoro-4-(phenylthio)benzoic acid (2.136 g, 97% Wt, 1 Eq, 6.333 mmol) in THF (10 mL) was added at −78° C. and the reaction mixture was stirred at −78° C. for 30 min. DMF (2.5 mL) was added, the reaction mixture was stirred at −78° C. for 30 min, then allowed to warm to RT and stirred for 1 h. The reaction mixture was carefully quenched with 1 M HCl (aq.) (50 mL) and then extracted with EtOAc (2×100 mL). The combined organic extracts were washed with brine (50 mL), dried (MgSO₄) and concentrated in vacuo to afford the crude product. The crude product was purified by chromatography to afford an inseparable mixture of 5-fluoro-2-formyl-4-(phenylthio)benzoic acid (1.732 g) and 3-fluoro-2-formyl-4-(phenylthio)benzoic acid (1.732 g) as a yellow oil. MS (ES⁺): 277 (M+H)⁺

### Step 4

A solution of 5-fluoro-2-formyl-4-(phenylthio)benzoic acid (1.430 g, 65% Wt, 1 Eq, 3.364 mmol) in EtOH (15 mL) was treated with hydrazine hydrate (327 mg, 320 μL, 35% Wt, 1.06 Eq, 3.57 mmol) and the resulting suspension was stirred at 80° C. for 4 h and then allowed to cool to RT and stirred for 18 h. Additional hydrazine hydrate (327 mg, 320 μL, 35% Wt, 1.06 Eq, 3.57 mmol) was added and the reaction mixture was heated to 85° C. for 21 h. The crude product (1.2 g) was purified by chromatography to afford a 2:1 inseparable mixture of 7-fluoro-6-(phenylthio)phthalazin-1(2H)-one and 5-fluoro-6-(phenylthio)phthalazin-1(2H)-one (1.033 g, 1.176 mmol) as a yellow solid. 50 mg of crude of the crude mixture was purified by chromatography on RP Flash C18 (4 g cartridge, 10-50% MeCN/0.1% formic acid in water) to afford a 4:1 mixture of 5-fluoro-6-(phenylthio)phthalazin-1(2H)-one (Intermediate 56) and 7-fluoro-6-(phenylthio)phthalazin-1(2H)-one (Intermediate 57) (12 mg). MS (ES⁺): 273 (M+H)⁺

### Intermediate 58—6-((1-(tetrahydro-2H-pyran-2-yl)-1H-pyrazol-4-yl)thio)phthalazin-1(2H)-one

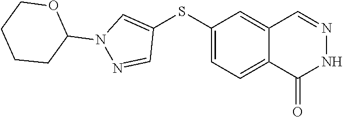 [SMILES: O=c1[nH]ncc2cc(Sc3cnn(C4CCCCO4)c3)ccc12]

A suspension of 6-mercaptophthalazin-1(2H)-one (1.26 g, 80% Wt, 1 Eq, 5.66 mmol), 4-iodo-1-(tetrahydro-2H-pyran-2-yl)-1H-pyrazole (1.82 g, 95% Wt, 1.1 Eq, 6.22 mmol), K₂CO₃ (1.56 g, 2.0 Eq, 11.3 mmol) and 1,10-phenanthroline (408 mg, 0.4 Eq, 2.26 mmol) in DMF (28.0 mL) was purged with N₂ for 10-15 min before adding copper(I) iodide (215 mg, 0.2 Eq, 1.13 mmol). The reaction mixture was purged for 10 min with N₂ then heated at 100° C. overnight. The reaction was cooled to RT and poured onto a mixture of ice and water leading to the formation of a precipitate that was filtered washed with water (50 mL). The dark brown gummy solid obtained was dissolved in DCM (250 mL) and was washed with half concentrated brine (200 mL). The organic was dried (MgSO₄), filtered and concentrated under reduced pressure to give a solid (1.87 g). The crude was combined with a crude mixture obtained from a separate experiment (1.63 g), dissolved in DCM (20 mL) and MeOH (2 mL), concentrated onto silica and purified by chromatography to afford the title compound (982.9 mg, 2.9 mmol) as a pale tan solid. MS (ES⁺): 195.1 (M+H)⁺

### Intermediate 59—1-(tetrahydro-2H-pyran-2-yl)-1H-pyrazole-4-thiol

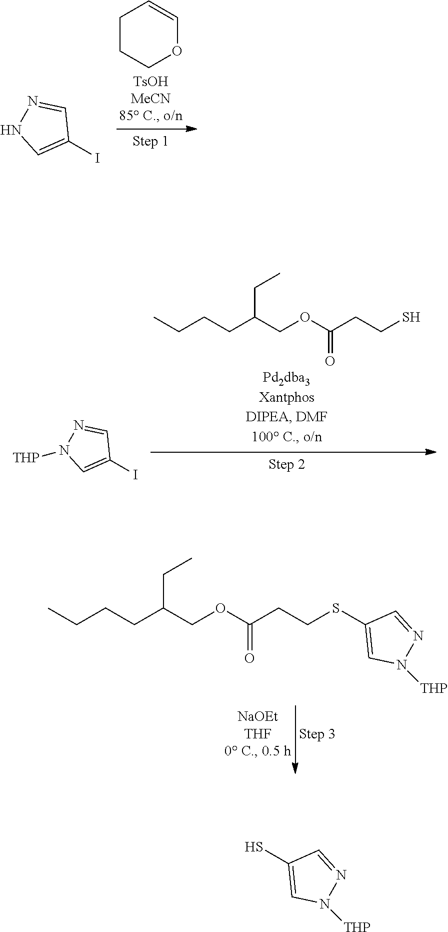

### Step 1

To a solution of 4-iodo-1H-pyrazole (80 g, 412 mmol) and 3,4-dihydro-2H-pyran (54 g, 618 mmol) in MeCN (1.2 L) was added TsOH (708 mg, 4.2 mmol) at RT. The reaction mixture was stirred at 85° C. for 16 h. After LCMS indicated the reaction was complete, The mixture was concentrated at 45° C. under reduced pressure. The residue was purified by flash column chromatography to give 4-iodo-1-(tetrahydro-2H-pyran-2-yl)-1H-pyrazole (100 g) as a white solid. MS (ES⁺): 279.2 (M+H)⁺

### Step 2

To a solution of 4-iodo-1-(tetrahydro-2H-pyran-2-yl)-1H-pyrazole (100 g, 360 mmol), 2-ethylhexyl 3-mercaptopropanoate (94 g, 432 mmol), Pd₂dba₃ (13 g, 14.4 mmol) and Xantphos (16.6 g, 28.8 mmol) in DMF (1 L) was added DIPEA (140 g, 1080 mmol) at RT. The reaction mixture was stirred at 100° C. for 16 h. After LCMS indicated the reaction was complete, the mixture was extracted with EtOAc (600 mL×3) and concentrated at 45° C. under reduced pressure. The residue was purified by flash column chromatography to give 2-ethylhexyl 3-((1-(tetrahydro-2H-pyran-2-yl)-1H-pyrazol-4-yl)thio)propanoate (116 g) as a yellow oil. MS (ES⁺): 369.3 (M+H)⁺

### Step 3

A mixture of 2-ethylhexyl 3-((1-(tetrahydro-2H-pyran-2-yl)-1H-pyrazol-4-yl)thio)propanoate (116 g, 315 mmol) in THF (2 L) was added NaOEt (252 mL, 630 mmol, 2.5 M of solution EtOH) at 0° C.

The reaction was stirred at 0° C. for 0.5 h. After LCMS indicated the reaction was complete, the reaction mixture was quenched with HCl (1M) until pH to 6. The mixture was extracted with EtOAc (600 mL×3). The organic layers were concentrated at 40° C. under reduced pressure and purified by flash column chromatography to give 1-(tetrahydro-2H-pyran-2-yl)-1H-pyrazole-4-thiol (45 g) as an oil, which was used in the next step without further purification. MS (ES⁺): 101.4 (M+H)⁺

### Intermediate 60—5-(chloromethyl)-2,3-dihydrofuro[3,2-b]pyridine

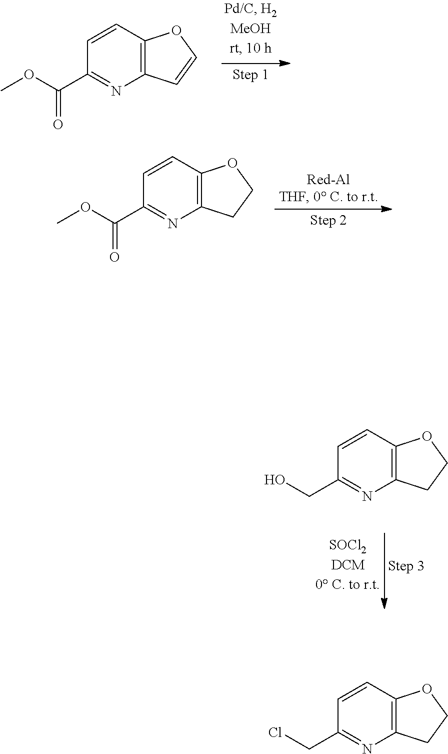

### Step 1

To a solution of methyl furo[3,2-b]pyridine-5-carboxylate (800 mg, 4.52 mmol) was added 10% Pd/C (320 mg, containing 50% water) in MeOH (10 mL), The mixture was stirred at 25° C. for 10 h under H₂. After LCMS indicated the reaction is completed, the mixture was dried over Na₂SO₄, filtered, and concentrated at 30° C. under reduced pressure the residue was purified by flash column chromatography to give methyl 2,3-dihydrofuro[3,2-b]pyridine-5-carboxylate (750 mg) as a yellow solid. MS (ES⁺): 180.3 (M+H)⁺

### Step 2

To the solution of methyl 2,3-dihydrofuro[3,2-b]pyridine-5-carboxylate (750 mg, 4.19 mmol) in THF (10 mL) was add Red-Al (2.42 g, 8.38 mmol, 70 wt % in toluene) at 0° C., and the reaction mixture was stirred at RT for 2 h. After LCMS indicated the reaction is completed, the mixture was added NH₄Cl (1 mL). The mixture was quenched over Na₂SO₄, filtered and concentrated at 30° C. under reduced pressure the residue was purified by flash column chromatography to give (2,3-dihydrofuro[3,2-b]pyridin-5-yl)methanol (360 mg) as a yellow solid. MS (ES⁺): 152.4 (M+H)⁺

### Step 3

To the solution of (2,3-dihydrofuro[3,2-b]pyridin-5-yl)methanol (140 mg, 0.93 mmol) in DCM (5 mL) was added drop-wise SOCl₂ (221 mg, 1.86 mmol) at 0° C., and the reaction mixture was stirred at RT for 2 h. After LCMS indicated the reaction was complete, the mixture was concentrated at 30° C. under reduced pressure to remove DCM and SOCl₂ to give 5-(chloromethyl)-2,3-dihydrofuro[3,2-b]pyridine (150 mg) as a yellow oil, which was used to the next step directly. MS (ES⁺): 170.4 (M+H)⁺

### Intermediate 61—1-(2-((tert-butyldimethylsilyl)oxy)ethyl)-3-iodo-1H-pyrazole

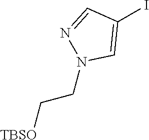 [SMILES: CC(C)(C)[Si](C)(C)OCCn1cc(I)cn1]

A mixture of 3-iodopyrazole (300 mg, 1 Eq, 1.55 mmol), (2-bromoethoxy)dimethyl-tert-butylsilane (407 mg, 366 μL, 1.1 Eq, 1.70 mmol) and potassium carbonate (321 mg, 1.5 Eq, 2.32 mmol) in MeCN (10.0 mL) was heated at 85° C. for 24 h. The reaction mixture was cooled to RT, diluted with DCM (10 mL) then filtered through celite with further wash with DCM (20 mL) and MeOH (10 mL). The residue (oil and solid) was partitioned between EtOAc (20 mL) and water (10 mL). The organic was washed with brine (10 mL×2), dried with MgSO₄ and concentrated under reduced pressure to afford crude product as a clear yellow liquid (536 mg) containing a mixture of regioisomers. The crude was dissolved in DCM (5 mL), concentrated onto silica and purified by chromatography to afford the title compound (108.5 mg) as a clear colourless oil. ¹H NMR (400 MHz, MeOD) δ 7.51 (d, J=2.3 Hz, 1H), 6.45 (d, J=2.3 Hz, 1H), 4.25 (t, J=5.1 Hz, 2H), 3.96 (t, J=5.1 Hz, 2H), 0.86 (s, 9H), −0.03 (s, 6H). MS (ES⁺): 353 (M+H)⁺

### Intermediate 62—5-(chloromethyl)furo[3,2-b]pyridine

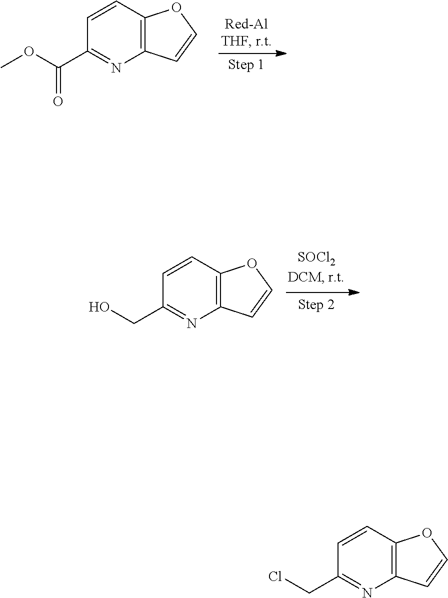

### Step 1

To a solution of methyl 2,3-dihydrofuro[3,2-b]pyridine-5-carboxylate (220 mg, 1.24 mmol) in THF (10 mL) was add Red-Al (536 mg, 1.86 mmol, 70 wt % in toluene) at 0° C., and the reaction mixture was stirred at room temperature for 2 hours. After LCMS indicated the reaction to be complete, to the mixture was added H₂O (1 mL). The mixture was dried over Na₂SO₄, filtered and concentrated at 30° C. under reduced pressure; the residue was purified by flash column chromatography (12 g, petroleum ether/ethyl acetate=100:00/50:50) to give furo[3,2-b]pyridin-5-ylmethanol (150 mg, 94.98% purity) as a yellow solid. MS (ES⁺): 150.3 (M+H)⁺

### Step 2

To the solution of furo[3,2-b]pyridin-5-ylmethanol (140 mg, 1.00 mmol) in DCM (5 mL) was drop-wised SOCl₂ (238 mg, 2.00 mmol) at 0° C., and the reaction mixture was stirred at room temperature for 2 hours. After LCMS indicated the reaction was completed, the mixture was concentrated at 30° C. under reduced pressure to remove DCM and most of over amounted SOCl₂ to give 5-(chloromethyl)furo[3,2-b]pyridine (150 mg, 94.83% purity) as a yellow oil, which was used to the next step directly. MS (ES⁺): 168.3 (M+H)⁺

### Intermediate 63—1-(tetrahydro-2H-pyran-2-yl)-1H-pyrazole-3-thiol

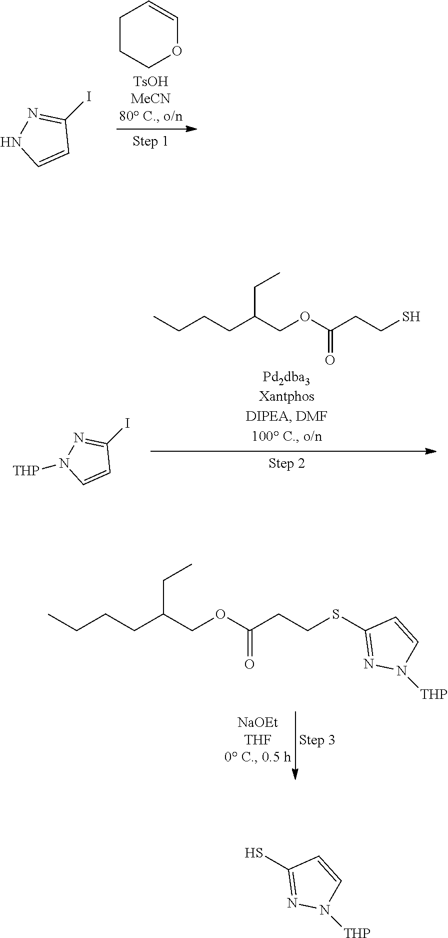

### Step 1

To a solution of 3-iodo-1H-pyrazole (5.0 g, 25.8 mmol) and 3,4-dihydro-2H-pyran (3.3 g, 38.7 mmol) in MeCN (50 mL) was added p-TsOH (43 mg, 0.3 mmol) at room temperature, and the reaction mixture was stirred at 80° C. for 16 hours. After LCMS indicated the reaction was completed, the reaction mixture was concentrated at 45° C. under reduced pressure. The residue was purified by flash column chromatography (25 g, petroleum ether/tert-Butyl methyl ether=100:0˜90:10) to give 3-iodo-1-(tetrahydro-2H-pyran-2-yl)-1H-pyrazole (6.6 g, 100% purity) as a yellow oil. MS (ES⁺): 279.1 (M+H)⁺

### Step 2

To a solution of 3-iodo-1-(tetrahydro-2H-pyran-2-yl)-1H-pyrazole (6.6 g, 23.7 mmol), 2-ethylhexyl 3-mercaptopropanoate (6.7 g, 30.8 mmol), Pd2dba3 (540 mg, 0.59 mmol) and Xantphos (685 mg, 1.19 mmol) in DMF (100 mL) was added DIPEA (9.2 g, 71.1 mmol) at room temperature. The reaction mixture was stirred at 100° C. for 16 hours. After LCMS indicated the reaction was completed, water (50 mL) was added and the mixture was extracted with EtOAc (50 mL×3). The combined organic layer was concentrated at 45° C. under reduced pressure. The residue was purified by flash column chromatography (120 g, petroleum ether/tert-Butyl methyl ether=100:00˜80:20) to give 2-ethylhexyl 3-((1-(tetrahydro-2H-pyran-2-yl)-1H-pyrazol-3-yl)thio)propanoate (7.1 g, 44.18% purity) as a yellow oil. MS (ES⁺): 369.3 (M+H)⁺

### Step 3

A mixture of 2-ethylhexyl 3-((1-(tetrahydro-2H-pyran-2-yl)-1H-pyrazol-3-yl)thio)propanoate (7.1 g, 19.3 mmol) in THF (150 mL) was added EtONa (15 mL, 2.5 M of solution EtOH) at 0° C. The reaction was stirred at 0° C. for 0.5 hours. After LCMS indicated the reaction was completed, the reaction mixture pH was adjusted with careful addition of aq. HCl (1M) until pH=6. The mixture was extracted with EtOAc (800 mL×3). The organic layers were concentrated at 40° C. under reduced pressure. The residue was purified by flash column chromatography (40 g, petroleum ether/tert-Butyl methyl ether=100:0-60:40) to give 1-(tetrahydro-2H-pyran-2-yl)-1H-pyrazole-3-thiol (2.8 g, 100% purity, 80% yield) as a yellow oil. MS (ES⁺): 185.3 (M+H)⁺

### Intermediate 64—5-(chloromethyl)-2,3-dihydrofuro[3,2-b]pyridine

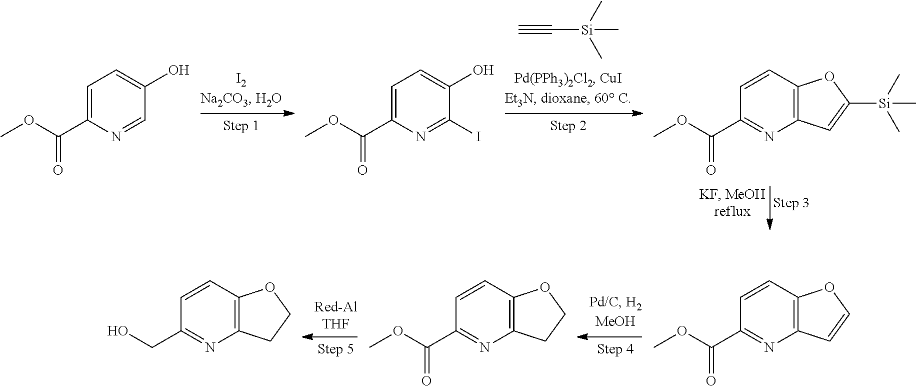

### Step 1

A mixture of methyl 5-hydroxypicolinate (225 g, 1470 mmol) and sodium carbonate (343 g, 3234 mmol) in H₂O (3000 mL) was added iodine (373 g, 1470 mmol) at 0° C. The reaction was stirred at room temperature for 20 hours. After LCMS indicated the reaction was completed, The reaction mixture was quenched with sat. aq sodium thiosulfate (200 mL) and then acidified with 1 M HCl (aq). The mixture was extracted with EtOAc (1000 mL×3). The organic layers were concentrated at 40° C. under reduced pressure to give methyl 5-hydroxy-6-iodopicolinate (290 g, 67.62% purity) as yellow oil, which was used directly into the next step without further purification. MS (ES⁺): 280.2 (M+H)⁺

### Step 2

To a solution of give methyl 5-hydroxy-6-iodopicolinate (290 g, 1039 mmol), Pd(PPh₃)₂Cl₂ (95 g, 135 mmol) and CuI (31 g, 166 mmol) in DOX (3000 mL) was added Et₃N (315 mg, 3117 mmol) at room temperature. The reaction mixture was stirred at room temperature for 5 minutes. ethynyltrimethylsilane (265 g, 2701 mmol) was added and the reaction mixture was stirred under nitrogen at 65° C. for 3 h and then allowed to cool to room temperature. After LCMS indicated the reaction was completed, the mixture was added water (1000 mL) and extracted with EtOAc (1000 mL×3). The organic layers were concentrated at 40° C. under reduced pressure. The residue was purified by flash column chromatography (120 g×8, petroleum ether/tert-Butyl methyl ether=100:00˜20:80) to give methyl 2-(trimethylsilyl)furo[3,2-b]pyridine-5-carboxylate (100 g, 84.18% purity) as a yellow solid. MS (ES⁺): 250.3 (M+H)⁺

### Step 3

To a solution of methyl 2-(trimethylsilyl)furo[3,2-b]pyridine-5-carboxylate (100 g, 400 mmol) in MeOH (1000 mL) was added KF (70 g, 1200 mmol) at room temperature and the reaction mixture was stirred at 80° C. for 5 hours. After LCMS indicated the reaction was completed, the mixture was concentrated at 40° C. under reduced pressure to remove MeOH. The residue was quenched with water (1000 mL) and extracted with DCM (3×1000 mL). The organic layer was washed with brine, dried over Na₂SO₄, filtered, and concentrated at 30° C. under reduced pressure. The residue was purified by flash column chromatography (120 g×8, petroleum ether/tert-Butyl methyl ether=100:00˜60:40) to give methyl furo[3,2-b]pyridine-5-carboxylate (55 g, 91.20% purity) as a yellow oil. MS (ES⁺): 178.4 (M+H)⁺

### Step 4

A solution of methyl furo[3,2-b]pyridine-5-carboxylate (55 g, 310 mmol) was added 20% Pd/C (11 g, 50% purity) in MeOH (600 mL), The mixture was stirred at 25° C. for 10 h under H2 atmosphere. After LCMS indicated the reaction was completed, the mixture was dried over Na2SO4, filtered and concentrated at 30° C. under reduced pressure the residue was purified by flash column chromatography (12 g, petroleum ether/ethyl acetate=100:00/50:50) to give methyl 2,3-dihydrofuro[3,2-b]pyridine-5-carboxylate (49 g, 92.82% purity) as a yellow solid. MS (ES⁺): 180.4 (M+H)⁺

### Step 5

To the solution of methyl 2,3-dihydrofuro[3,2-b]pyridine-5-carboxylate (49 g, 273 mmol) in THF (300 mL) was add Red-Al (150 g, 546 mmol, 70 wt % in toluene) at 0° C., and the reaction mixture was stirred at room temperature for 2 hours. After LCMS indicated the reaction was completed, the mixture was added NH₄Cl (1000 mL). The mixture was dried over Na₂SO₄, filtered and concentrated at 30° C. under reduced pressure the residue was purified by flash column chromatography (120 g, petroleum ether/ethyl acetate=100:00/50:50) to give the desired compound (22 g, 83% purity) as a yellow solid. MS (ES⁺): 152.4 (M+H)⁺

### Intermediate 65—3-(chloromethyl)-5-methyl-1-((2-(trimethylsilyl)ethoxy)methyl)-1H-pyrazole

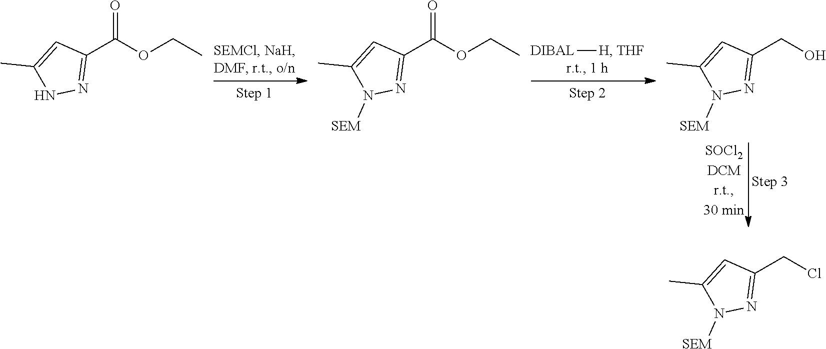

### Step 1

A solution of ethyl 5-methyl-1H-pyrazole-3-carboxylate (530 mg, 3.44 mmol) in DMF (6 mL) was added NaH (130 mg, 3.78 mmol, 70% wt) at 0° C. and stirred at room temperature for 30 minutes. To the reaction mixture was added SEMCl (803 mg, 4.82 mmol) and stirred at room temperature overnight. After LCMS indicated the reaction was completed, the reaction mixture was quenched with sat. aq NH₄Cl (20 mL) and extracted with EtOAc (50 mL×3). The combined organic layers were washed with sat. aq. NH₄Cl (30 mL×2), concentrated at 40° C. under reduced pressure and purified by flash column chromatography (12 g, petroleum ether/tert-butyl methyl ether=100:00˜80:20) to give ethyl 5-methyl-1-((2-(trimethylsilyl)ethoxy)methyl)-1H-pyrazole-3-carboxylate (520 mg, 95% purity) as light yellow oil. MS (ES⁺): 285.4 (M+H)⁺. ¹H NMR (400 MHz, CDCl₃) δ: 6.73 (s, 1H), 5.82 (s, 2H), 4.38 (q, J=7.2 Hz, 2H), 3.62 (t, J=8.4 Hz, 2H), 2.33 (s, 3H), 0.94 (t, J=8.4 Hz, 2H).

### Step 2

To a solution of ethyl 5-methyl-1-((2-(trimethylsilyl)ethoxy)methyl)-1H-pyrazole-3-carboxylate (440 mg, 1.55 mmol) in THF (6 mL) was cooled to 0° C., and was added DIBAL-H (6 mL, 6.0 mmol, 1 M in THF). The reaction mixture under nitrogen protection was stirred at room temperature for 1 hour. After LCMS indicated the reaction was completed, the reaction mixture was quenched with Na₂SO₄·10H₂O (390 mg, 1.21 mmol), stirred for 30 minutes and was added Na₂SO₄ for another 30 minutes. The suspension was filtered and the filtered cake was washed with MeOH/DCM (1:5). The combined organic layers were concentrated at 40° C. under reduced pressure and purified by flash column chromatography (12 g, petroleum ether/tert-butyl methyl ether=100:00˜20:80) to give (5-methyl-1-((2-(trimethylsilyl)ethoxy)methyl)-1H-pyrazol-3-yl)methanol (310 mg, 100% purity) as white solid. MS (ES⁺): 243.3 (M+H)⁺

### Step 3

A mixture of (5-methyl-1-((2-(trimethylsilyl)ethoxy)methyl)-1H-pyrazol-3-yl)methanol (310 mg, 1.3 mmol) in DCM (4 mL) was added SOCl₂ (238 mg, 2.0 mmol). The reaction mixture was stirred at room temperature for 30 minutes. After LCMS indicated the reaction was completed, the mixture was concentrated to remove SOCl₂ and DCM to give the crude material. The product was used without further purification.

### Example 1—2-((1-cyclopropyl-1H-pyrazol-3-yl)methyl)-6-((4-methoxyphenyl)sulfonyl)phthalazin-1(2H)-one

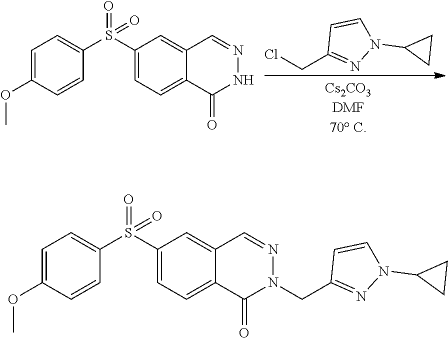

A mixture of 6-((4-methoxyphenyl)sulfonyl)phthalazin-1(2H)-one (Intermediate 1, 50 mg, 1 Eq, 0.16 mmol) and cesium carbonate (0.15 g, 3 Eq, 0.47 mmol) in DMF (1 mL) was stirred at 70° C. for 45 minutes. 3-(chloromethyl)-1-cyclopropyl-1H-pyrazole hydrochloride (Intermediate 6, 31 mg, 1 Eq, 0.16 mmol) was added and the mixture was allowed to cool to RT and stirred overnight. Water and DCM were added and the layers separated. The organic layer was washed with brine, then absorbed on silica. The crude product was purified by chromatography on silica gel. The product was dissolved in DCM and washed with water, brine, dried (MgSO₄) and concentrated in vacuo to afford the title compound (27.3 mg) as a pale yellow solid. ¹H NMR (DMSO-d6) δ: 8.63 (d, 1H), 8.59 (s, 1H), 8.41 (d, 1H), 8.27 (dd, 1H), 8.00-7.91 (m, 2H), 7.64 (d, 1H), 7.21-7.12 (m, 2H), 6.06 (d, 1H), 5.23 (s, 2H), 3.83 (s, 3H), 3.67-3.56 (m, 1H), 1.00-0.85 (m, 4H). MS (ES⁺): 437 (M+H)⁺

### Example 2—2-(indolin-4-ylmethyl)-6-((4-methoxyphenyl)sulfonyl)phthalazin-1(2H)-one

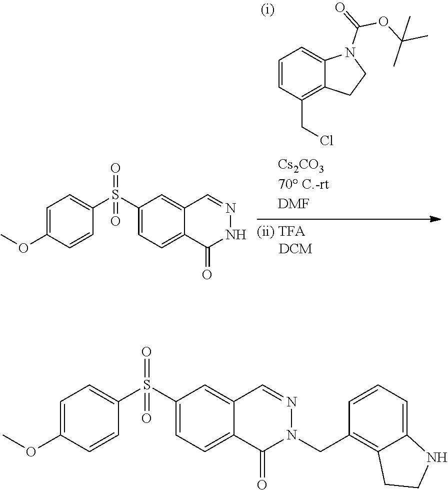 [SMILES: CC(C)(C)OC(=O)N1CCc2c(CCl)cccc21 ; COc1ccc(S(=O)(=O)c2ccc3c(=O)[nH]ncc3c2)cc1 ; COc1ccc(S(=O)(=O)c2ccc3c(=O)n(Cc4cccc5c4CCN5)ncc3c2)cc1]

To a stirred solution of 6-((4-methoxyphenyl)sulfonyl)phthalazin-1(2H)-one (Intermediate 1, 60 mg, 1 Eq, 0.19 mmol) and cesium carbonate (68 mg, 1.1 Eq, 0.21 mmol) in dry DMF (6 mL) was added tert-butyl 4-(chloromethyl)indoline-1-carboxylate (Intermediate 4, 90 mg, 85% Wt, 1.5 Eq, 0.28 mmol). The reaction mixture was stirred for 18 h at 20 C. The reaction was allowed to cool to RT, then diluted with DCM (10 mL) and washed with water (20 mL). The organic phase was collected, dried (phase separator) and evaporated under reduced pressure. The crude was dissolved in DCM (2 mL) and treated with TFA (0.22 g, 0.15 mL, 10 Eq, 1.9 mmol). The resulting mixture was stirred for few minutes before being washed with a sat. solution of NaHCO₃ (2 mL). The organic phase was collected, dried (phase separator) and evaporated under reduced pressure. The crude product was dissolved in DMSO (0.6 mL), filtered and purified by reversed phase preparative HPLC (Acidic method (A)) The relevant fractions were evaporated in a Genevac, yielding the title compound (6 mg) as a pale yellow solid. ¹H NMR (400 MHz, DMSO-d6) δ 8.63 (d, J=1.8 Hz, 1H), 8.61 (s, 1H), 8.42 (d, J=8.4 Hz, 1H), 8.27 (dd, J=8.5, 1.9 Hz, 1H), 7.99-7.93 (m, 2H), 7.19-7.14 (m, 2H), 6.80 (t, J=7.7 Hz, 1H), 6.37 (d, J=7.7 Hz, 1H), 6.33 (d, J=7.6 Hz, 1H), 5.50 (s, 1H), 5.20 (s, 2H), 3.83 (s, 3H), 3.44-3.36 (m, 2H), 2.92 (t, J=8.5 Hz, 2H). (ES⁺): 404 (M+H)⁺. MS (ES⁺): 448.2 (M+H)⁺

### Example 3—2-((2-hydroxypyridin-4-yl)methyl)-6-((4-methoxyphenyl)sulfonyl)phthalazin-1(2H)-one

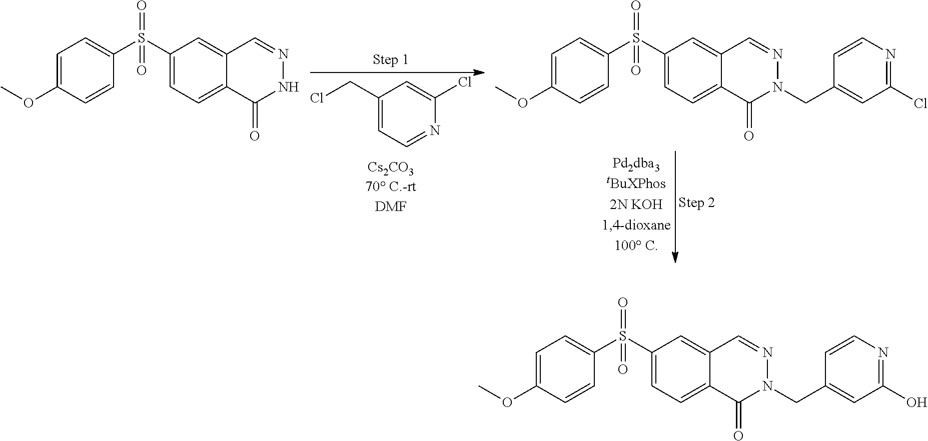

### Step 1

A mixture of 6-((4-methoxyphenyl)sulfinyl)phthalazin-1(2H)-one-6-((4-methoxyphenyl)sulfonyl)phthalazin-1(2H)-one (Intermediate 1, 100 mg, 66% Wt, 1 Eq, 215 μmol) and cesium carbonate (210 mg, 3 Eq, 645 μmol) in DMF (1 mL) was stirred at 70° C. for 45 minutes. 2-chloro-4-(chloromethyl)pyridine (90.6 mg, 2.6 Eq, 559 μmol) was added and the mixture was allowed to cool to RT and stirred for 3 days. Water and DCM were added and the layers separated. The organic layer was washed with brine, then absorbed on silica. The crude product was purified by chromatography on silica gel to afford 2-((2-chloropyridin-4-yl)methyl)-6-((4-methoxyphenyl)sulfonyl)phthalazin-1(2H)-one (51 mg) as a white solid. MS (ES⁺): 442/444 (M+H)⁺.

### Step 2

N₂ was bubbled through a mixture of 2-((2-chloropyridin-4-yl)methyl)-6-((4-methoxyphenyl)sulfonyl)phthalazin-1(2H)-one (25 mg, 1 Eq, 57 μmol) and 2N KOH (0.28 mL, 2 molar, 10 Eq, 0.57 mmol) in 1,4-Dioxane (0.3 mL) for 5 minutes. Pd₂dba₃ (2.6 mg, 0.05 Eq, 2.8 μmol) and tBuXPhos (7.2 mg, 0.3 Eq, 17 μmol) were added and the mixture was stirred under N₂ at 100° C. for 2 h, then allowed to cool to RT and stirred overnight. 1N HCl (0.3 mL), water and DCM were added and the layers separated. The organic layer was washed with brine, then absorbed on silica. The crude product was purified by chromatography on silica gel to the title compound (9.2 mg) as a pale tan solid. ¹H NMR (DMSO-d6) δ: 11.48 (s, 1H), 8.66 (d, 1H), 8.65 (s, 1H), 8.42 (d, 1H), 8.29 (dd, 1H), 8.00-7.92 (m, 2H), 7.32-7.25 (m, 1H), 7.23-7.13 (m, 2H), 6.08-6.02 (m, 2H), 5.14 (s, 2H), 3.84 (s, 3H). MS (ES⁺): 424 (M+H)⁺

### Example 4—6-((4-methoxyphenyl)sulfonyl)-2-((1-methyl-2-oxo-1,2-dihydropyridin-3-yl)methyl)phthalazin-1(2H)-one

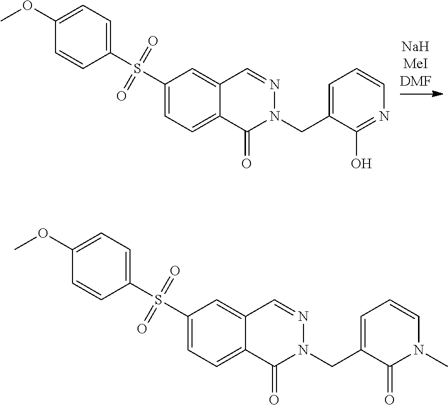

Sodium hydride (2.8 mg, 60% Wt, 2 Eq, 71 μmol) was added to a stirred solution of 2-((2-hydroxypyridin-3-yl)methyl)-6-((4-methoxyphenyl)sulfonyl)phthalazin-1(2H)-one (Example 56, 15 mg, 1 Eq, 35 μmol) in dry DMF (2 mL). The reaction mixture was stirred for 5 minutes, then methyl iodide (10 mg, 4.4 μL, 2 Eq, 71 μmol) was added. The resulting mixture was stirred at RT for 2 h. The reaction mixture was diluted with EtOAc (10 mL) and washed with water (10 mL), and brine (10 mL). The organic phase was collected, dried over MgSO₄ and concentrated under reduced pressure. The crude product was purified by chromatography on silica gel to afford the title compound (15 mg) as a pale brown solid. ¹H NMR (400 MHz, DMSO-d6) δ 8.65 (d, J=1.9 Hz, 1H), 8.61 (s, 1H), 8.42 (d, J=8.4 Hz, 1H), 8.28 (dd, J=8.4, 1.9 Hz, 1H), 7.99-7.93 (m, 2H), 7.64 (dd, J=6.7, 2.0 Hz, 1H), 7.20-7.14 (m, 2H), 7.01 (dd, J=6.9, 1.9 Hz, 1H), 6.10 (t, J=6.8 Hz, 1H), 5.10 (s, 2H), 3.84 (s, 3H), 3.44 (s, 3H). MS (ES⁺): 438 (M+H)⁺

### Example 5—2-((1-(2-hydroxyethyl)-2-oxo-1,2-dihydropyridin-3-yl)methyl)-6-(4-methoxyphenylsulfonyl)phthalazin-1(2H)-one

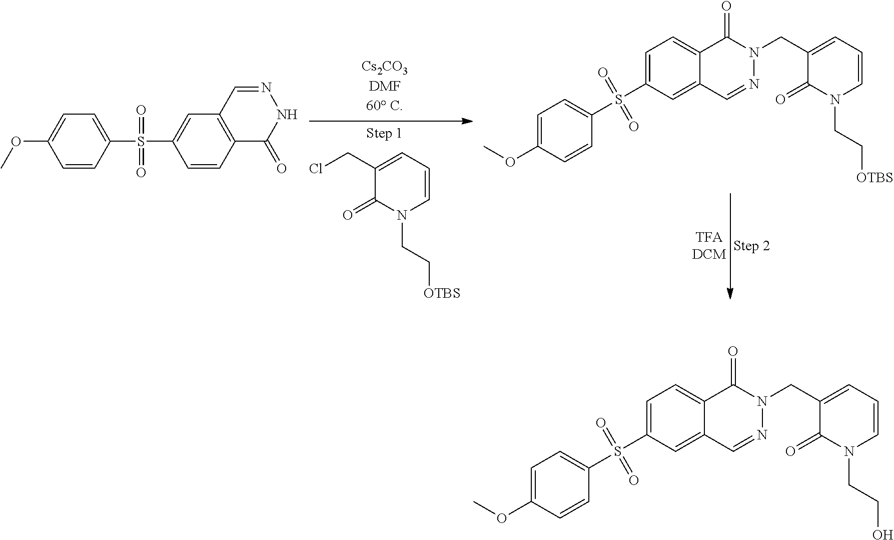

### Step 1

To the solution of 1-(2-(tert-butyldimethylsilyloxy)ethyl)-3-(chloromethyl)pyridin-2(1H)-one (Intermediate 30, 80 mg, 0.27 mmol), Cs₂CO₃ (264 mg, 0.81 mmol) in DMF (5 mL) was added 6-(4-methoxyphenylsulfonyl)phthalazin-1(2H)-one (Intermediate 1, 161 mg, 1.0 mmol) at 60° C. overnight. After LCMS indicated the reaction completed, the reaction mixture was quenched with water (10 mL) and filtered. The cake was dissolved EtOAc (5 mL), and washed with brine, dried over Na₂SO₄ and filtered. The filtrate was concentrated at 30° C. under reduced pressure, the residue was triturated with petroleum ether/ethyl acetate=1/1, filtered and dried at 30° C. under reduced pressure to give 2-((1-(2-(tert-butyldimethylsilyloxy)ethyl)-2-oxo-1,2-dihydropyridin-3-yl)methyl)-6-(4methoxyphenylsulfonyl)phthalazin-1(2H)-one (75 mg) as a yellow solid. MS (ES⁺): 582.0 (M+H)⁺

### Step 2

To a solution of 2-((1-(2-(tert-butyldimethylsilyloxy)ethyl)-2-oxo-1,2-dihydropyridin-3-yl)methyl)-6-(4-methoxyphenylsulfonyl)phthalazin-1(2H)-one (75 mg, 0.13 mmol) in TFA/DCM=(1/1) (3 mL), and the reaction mixture was stirred at RT for 3 h. After LCMS indicated the reaction completed, to the mixture was added DCM (5 mL), the combined organic layer was washed by aq. NaHCO₃, separated and extracted with DCM (3×5 mL). dried over Na₂SO₄, filtered and concentrated at 45° C. under reduced pressure. The residue was purified by prep-HPLC (Basic Method (B)) The relevant fractions were concentrated at 42° C. under reduced pressure to remove MeCN, and the residue was lyophilized to give the title compound (37.91 mg) as white solid. ¹H NMR (400 MHz, DMSO-d6) δ: 8.65 (d, J=1.6 Hz, 1H), 8.62 (s, 1H), 8.42 (d, J=8.4 Hz, 1H), 8.28 (dd, J=2.0 Hz, J₂=8.4 Hz, 1H), 7.97 (d, J=8.8 Hz, 2H), 7.54 (dd, J=1.6 Hz, J₂=6.8 Hz, 1H), 7.18 (d, J=9.2 Hz, 2H), 6.98 (d, J=5.6 Hz, 1H), 6.09 (t, J=6.8 Hz, 1H), 5.10 (s, 2H), 4.88 (t, J=5.2 Hz, 1H), 3.96 (t, J=5.6 Hz, 2H), 3.84 (s, 3H), 3.63 (q, J=5.6 Hz, 2H). MS (ES⁺): 468.0 (M+H)⁺.

### Example 6—2-((1H-pyrazol-3-yl)methyl)-6-((4-methoxyphenyl)sulfonyl)phthalazin-1(2H)-one

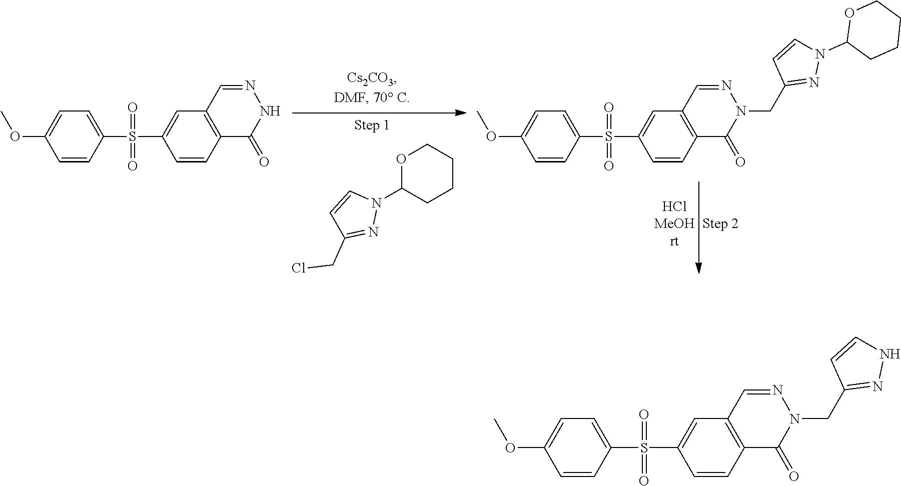 [SMILES: COc1ccc(S(=O)(=O)c2ccc3c(=O)[nH]ncc3c2)cc1 ; ClCc1ccn(C2CCCCO2)n1 ; COc1ccc(S(=O)(=O)c2ccc3c(=O)n(Cc4ccn(C5CCCCO5)n4)ncc3c2)cc1 ; COc1ccc(S(=O)(=O)c2ccc3c(=O)n(Cc4cc[nH]n4)ncc3c2)cc1]

### Step 1

A mixture of 6-((4-methoxyphenyl)sulfonyl)phthalazin-1(2H)-one (Intermediate 1, 100 mg, 1 Eq, 316 μmol) and cesium carbonate (206 mg, 2 Eq, 632 μmol) in DMF (1 mL) was stirred at 70° C. for 45 minutes. 3-(chloromethyl)-1-(tetrahydro-2H-pyran-2-yl)-1H-pyrazole (Intermediate 5, 63.4 mg, 1 Eq, 316 μmol) was added and the mixture was allowed to cool to RT and stirred overnight. Water and DCM were added and the layers separated. The organic layer was washed with brine, then absorbed on silica. The crude product was purified by chromatography on silica gel to afford 6-((4-methoxyphenyl)sulfonyl)-2-((1-(tetrahydro-2H-pyran-2-yl)-1H-pyrazol-3-yl)methyl)phthalazin-1(2H)-one (50 mg) as a pale yellow glass. MS (ES⁺): 481 (M+H)⁺.

### Step 2

HCl (4N in dioxane) (56 mg, 0.38 mL, 4 molar, 15 Eq, 1.5 mmol) was added to a solution of 6-((4-methoxyphenyl)sulfonyl)-2-((1-(tetrahydro-2H-pyran-2-yl)-1H-pyrazol-3-yl)methyl)phthalazin-1(2H)-one (50 mg, 98% Wt, 1 Eq, 0.10 mmol) in MeOH (0.5 mL) and the mixture was stirred for 1 h, then concentrated in vacuo. Sat. NaHCO₃ and DCM were added and the layers separated. The organic layer was washed with brine, then absorbed on silica. The crude product was purified by chromatography on silica gel to afford the title product (29.8 mg) as a white solid. ¹H NMR (DMSO-d6) δ: 12.64 (s, 1H), 8.63 (d, J=1.8 Hz, 1H), 8.59 (s, 1H), 8.42 (d, J=8.4 Hz, 1H), 8.27 (dd, J=8.4, 1.9 Hz, 1H), 7.99-7.91 (m, 2H), 7.61 (s, 1H), 7.21-7.12 (m, 2H), 6.12 (s, 1H), 5.29 (s, 2H), 3.83 (s, 3H). MS (ES⁺): 397 (M+H)⁺.

### Example 7—2-((5-hydroxypyridin-3-yl)methyl)-6-(phenylsulfonyl)phthalazin-1(2H)-one

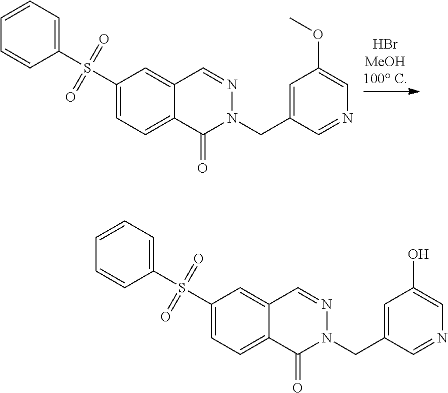 [SMILES: COc1cncc(Cn2ncc3cc(S(=O)(=O)c4ccccc4)ccc3c2=O)c1 ; O=c1c2ccc(S(=O)(=O)c3ccccc3)cc2cnn1Cc1cncc(O)c1]

2-((5-methoxypyridin-3-yl)methyl)-6-(phenylsulfonyl)phthalazin-1(2H)-one (Example 31,100 mg, 1 Eq, 245 μmol) was treated with HBr (4.14 g, 2.78 mL, 48% Wt, 100 Eq, 24.5 mmol). The reaction mixture was stirred for 72 h at 100° C. The reaction mixture was allowed to cool to RT, then diluted with aq. NaHCO₃ until pH-6 and extracted with EtOAc (2×10 mL). The organic layer was washed with water (10 mL), and brine (10 mL). The organic phase was collected, dried over MgSO₄ and concentrated under reduced pressure. The crude product was purified by chromatography on silica gel to afford the title compound (10 mg) as a grey solid. ¹H NMR (400 MHz, DMSO-d6) δ 9.89 (s, 1H), 8.70 (d, J=1.8 Hz, 1H), 8.65 (s, 1H), 8.44 (d, J=8.4 Hz, 1H), 8.32 (dd, J=8.4, 1.9 Hz, 1H), 8.03 (dt, J=6.3, 1.3 Hz, 4H), 7.78-7.71 (m, 1H), 7.70-7.62 (m, 2H), 7.08 (t, J=2.3 Hz, 1H), 5.28 (s, 2H). MS (ES⁺): 394 (M+H)⁺.

### Example 8—2-(2-methoxy-1-(6-methoxypyridin-3-yl)ethyl)-6-(phenylsulfonyl)phthalazin-1(2H)-one

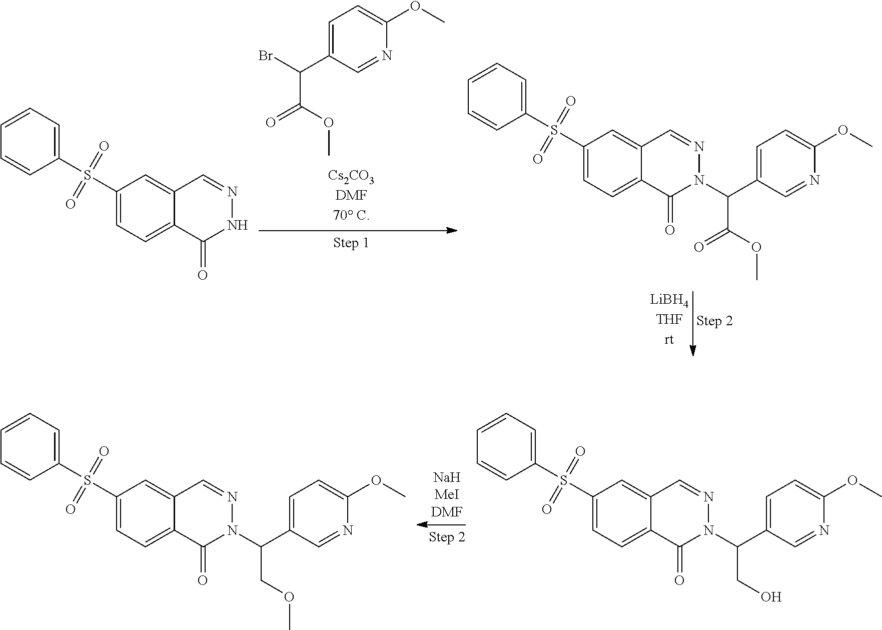 [SMILES: O=c1[nH]ncc2cc(S(=O)(=O)c3ccccc3)ccc12 ; COC(=O)C(Br)c1ccc(OC)nc1 ; COC(=O)C(c1ccc(OC)nc1)n1ncc2cc(S(=O)(=O)c3ccccc3)ccc2c1=O ; COCC(c1ccc(OC)nc1)n1ncc2cc(S(=O)(=O)c3ccccc3)ccc2c1=O ; COc1ccc(C(CO)n2ncc3cc(S(=O)(=O)c4ccccc4)ccc3c2=O)cn1]

### Step 1

To a stirred solution of 6-(phenylsulfonyl)phthalazin-1(2H)-one (Intermediate 2, 0.180 g, 1.0 Eq, 629 μmol) and cesium carbonate (451 mg, 2.2 Eq, 1.38 mmol) in dry DMF (4.0 mL) under N₂ at 70° C. was added methyl 2-bromo-2-(6-methoxypyridin-3-yl)acetate (Intermediate 11, 180 mg, 1.1 Eq, 692 μmol). The reaction mixture was stirred for 5 h. The reaction was allowed to cool to RT, diluted with water (10 mL) and extracted with DCM (3×15 mL). The combined organic layers were dried over MgSO₄ and evaporated under reduced pressure. The crude product was purified by chromatography on silica gel to afford methyl 2-(6-methoxypyridin-3-yl)-2-(1-oxo-6-(phenylsulfonyl)phthalazin-2(1H)-yl)acetate (0.190 g) as a white solid. ¹H NMR (400 MHz, DMSO-d6) δ 8.70 (d, J=1.8 Hz, 1H), 8.64 (s, 1H), 8.44 (d, J=8.4 Hz, 1H), 8.33 (dd, J=8.5, 1.9 Hz, 1H), 8.23 (d, J=2.5 Hz, 1H), 8.06-7.99 (m, 2H), 7.79 (dd, J=8.7, 2.5 Hz, 1H), 7.76-7.71 (m, 1H), 7.71-7.62 (m, 2H), 6.83 (d, J=8.6 Hz, 1H), 6.75 (s, 1H), 3.84 (s, 3H), 3.69 (s, 3H). MS (ES⁺): 466.2 (M+H)⁺.

### Step 2

A solution of methyl 2-(6-methoxypyridin-3-yl)-2-(1-oxo-6-(phenylsulfonyl)phthalazin-2(1H)-yl)acetate (0.190 g, 1 Eq, 408 μmol) in THF (4.0 mL) was treated with lithium borohydride (10.7 mg, 245 μL, 2.00 molar, 1.2 Eq, 490 μmol) dropwise. The reaction mixture was stirred at RT for 4 h and then quenched with sat. aq. NaHCO₃ (5 mL). The mixture was extracted with EtOAc (2×10 mL) and the combined organic extracts were washed with brine (10 mL), dried (MgSO₄) and concentrated in vacuo. The crude product was purified by chromatography on silica gel to afford 2-(2-hydroxy-1-(6-methoxypyridin-3-yl)ethyl)-6-(phenylsulfonyl)phthalazin-1(2H)-one (0.049 g) as a white solid. ¹H NMR (400 MHz, DMSO-d6) δ 8.71-8.65 (m, 2H), 8.42 (d, J=8.5 Hz, 1H), 8.30 (d, J=8.5 Hz, 1H), 8.18 (d, J=2.4 Hz, 1H), 8.01 (d, J=7.8 Hz, 2H), 7.77-7.69 (m, 2H), 7.69-7.61 (m, 2H), 6.76 (d, J=8.6 Hz, 1H), 6.13 (dd, J=9.1, 5.5 Hz, 1H), 5.00 (t, J=5.7 Hz, 1H), 4.27-4.16 (m, 1H), 3.95-3.85 (m, 1H), 3.80 (s, 3H). MS (ES⁺): 438.2 (M+H)⁺.

### Step 3

To a stirred solution of 2-(2-hydroxy-1-(6-methoxypyridin-3-yl)ethyl)-6-(phenylsulfonyl)phthalazin-1(2H)-one (0.035 g, 1 Eq, 80 μmol) in dry DMF (2 mL) under a N₂ at 0° C. was added NaH (4.8 mg, 60% Wt, 1.5 Eq, 0.12 mmol). The reaction mixture was stirred for 10 minutes, then iodomethane (23 mg, 10 μL, 2.0 Eq, 0.16 mmol) was added drop-wise. The resulting yellow suspension was stirred for 2 h while warming-up to RT. The suspension was diluted with water (5 mL) and extracted with EtOAc (2×10 mL). The organic phase was collected, washed with brine (100 mL), dried (MgSO₄) and evaporated under reduced pressure to afford the title compound (0.020 g) as a white solid. ¹H NMR (400 MHz, DMSO-d6) δ 8.69 (s, 2H), 8.42 (d, J=8.4 Hz, 1H), 8.30 (dd, J=8.4, 1.8 Hz, 1H), 8.21 (d, J=2.5 Hz, 1H), 8.01 (d, J=7.3 Hz, 2H), 7.79-7.70 (m, 2H), 7.69-7.60 (m, 2H), 6.77 (d, J=8.6 Hz, 1H), 6.31 (dd, J=9.4, 5.4 Hz, 1H), 4.20 (t, J=9.8 Hz, 1H), 3.85 (dd, J=10.1, 5.5 Hz, 1H), 3.80 (s, 3H), 3.25 (s, 3H). MS (ES⁺): 452.1 (M+H)⁺.

### Example 9—3-((1-oxo-6-(phenylsulfonyl)phthalazin-2(1H)-yl)methyl)picolinamide

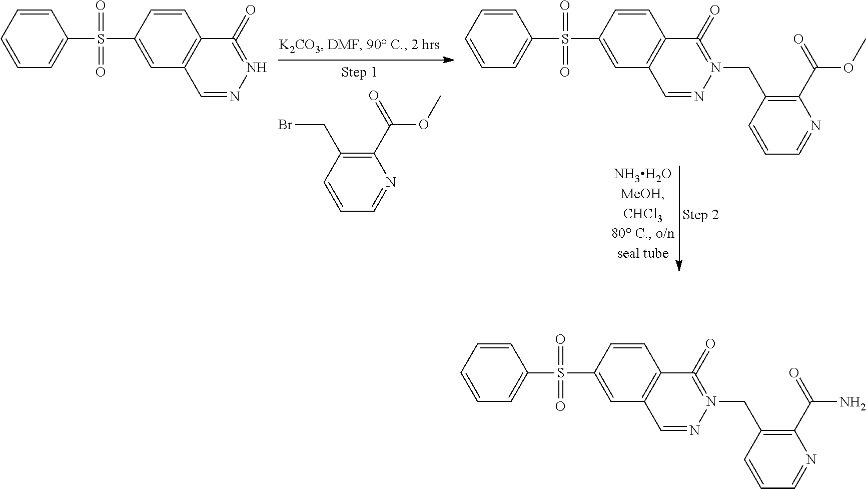

### Step 1

To a solution of 6-(phenylsulfonyl)phthalazin-1(2H)-one (Intermediate 2, 200 mg, 0.70 mmol) and K₂CO₃ (179 mg, 1.4 mmol) in DMF (5 mL) was added methyl 3-(bromomethyl)picolinate (Intermediate 16, 240 mg, 1.05 mmol) at RT, and the reaction mixture was stirred at 90° C. for 2 h. After LCMS indicated the reaction completed, the reaction mixture was filtered. The mixture was added EtOAc (10 mL), the combined organic layer was washed by brine, separated and extracted with EtOAc (3×10 mL). dried over Na₂SO₄, filtered and concentrated at 45° C. under reduced pressure, and the residue was purified by flash column chromatography to give methyl 3-((1-oxo-6-(phenylsulfonyl)phthalazin-2(1H)-yl)methyl)picolinate (180 mg) as a yellow solid. MS (ES⁺): 436.0 (M+H)⁺.

### Step 2

In a sealed-tube of 3-((1-oxo-6-(phenylsulfonyl)phthalazin-2(1H)-yl)methyl)picolinate (180 mg, 0.41 mmol) in MeOH (3 mL) and CHCl₃ (3 mL), 28% aqueous ammonium hydroxide solution (10 mL) was added and the reaction mixture was stirred at 80° C. overnight. After LCMS indicated the reaction completed, the reaction mixture was concentrated at 40° C. under reduced pressure. The residue was purified by prep-HPLC (Column: Waters X-SELECT C18 OBD 10 μm 19*250 mm; Flow Rate: 20 mL/min; solvent system: MeCN/(10 mmol/L NH₄HCO₃/water) gradient: MeCN: 40%-95%; collection wavelength: 214 nm). The relevant fractions were concentrated at 42° C. under reduced pressure to remove MeCN, and the residue was lyophilized to give the title compound (50.39 mg) as white solid. ¹H NMR (400 MHz, DMSO-d6) δ: 8.75 (d, J=1.6 Hz, 1H), 8.67 (s, 1H), 8.52 (t, J=2.8 Hz, 1H), 8.44 (d, J=7.6 Hz, 1H), 8.34 (dd, J=2.0 Hz, J₂=8.4 Hz, 1H), 8.14 (s, 1H), 8.07-8.04 (m, 2H), 7.78-7.73 (m, 1H), 7.71-7.66 (m, 1H), 7.41 (d, J=3.2 Hz, 1H), 5.77 (s, 2H). MS (ES⁺): 420.9 (M+H)⁺.

### Example 10—2-((1-(2-hydroxyethyl)-1H-pyrazol-3-yl)methyl)-6-(phenylsulfonyl)phthalazin-1(2H)-one

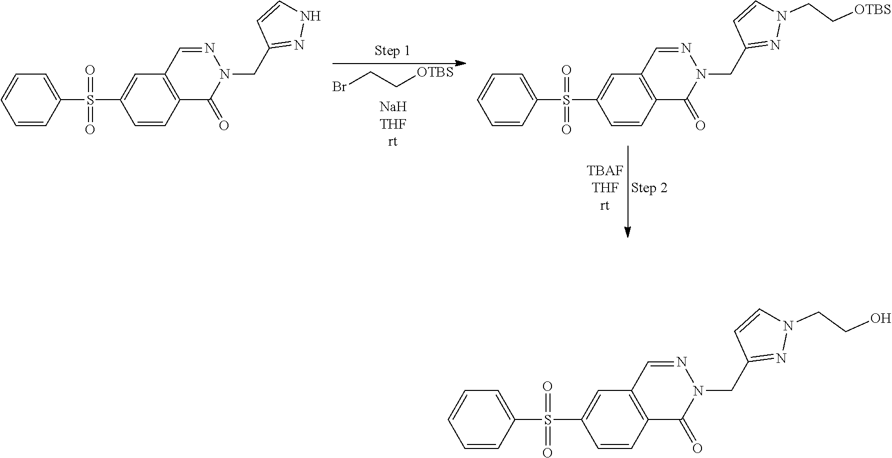 [SMILES: O=c1c2ccc(S(=O)(=O)c3ccccc3)cc2cnn1Cc1cc[nH]n1 ; CC(C)(C)[Si](C)(C)OCCBr ; CC(C)(C)[Si](C)(C)OCCn1ccc(Cn2ncc3cc(S(=O)(=O)c4ccccc4)ccc3c2=O)n1 ; O=c1c2ccc(S(=O)(=O)c3ccccc3)cc2cnn1Cc1ccn(CCO)n1]

### Step 1

A stirred solution of 2-((1H-pyrazol-3-yl)methyl)-6-(phenylsulfonyl)phthalazin-1(2H)-one (Example 68,100 mg, 98% Wt, 1 Eq, 267 μmol) in THF (2 mL) was treated with sodium hydride (60 wt % in mineral oil) (16.0 mg, 60% Wt, 1.5 Eq, 401 μmol). The reaction mixture was stirred for 30 minutes, then a solution of (2-bromoethoxy)(tert-butyl)dimethylsilane (69.1 mg, 62.0 μL, 99% Wt, 1.07 Eq, 286 μmol) in THF (1 mL) was added dropwise. The reaction mixture was stirred for 24 h. sodium iodide (8.02 mg, 0.20 Eq, 53.5 μmol), cesium carbonate (87.1 mg, 1 Eq, 267 μmol) and (2-bromoethoxy)(tert-butyl)dimethylsilane (69.1 mg, 62.0 μL, 99% Wt, 1.07 Eq, 286 μmol) were added and the reaction mixture was stirred for 24 h. The reaction mixture was partitioned between EtOAc (20 mL) and water (20 mL). The organic layer was collected and the aqueous was extracted with EtOAc (3×10 mL). The combined organic extracts were washed with brine (20 mL), dried (MgSO₄) and concentrated in vacuo to afford the crude product. The crude product was purified by chromatography on silica gel to afford 2-((1-(2-((tert-butyldimethylsilyl)oxy)ethyl)-1H-pyrazol-3-yl)methyl)-6-(phenylsulfonyl)phthalazin-1(2H)-one (32 mg) as a yellow solid. MS (ES⁺): 525 (M+H)⁺.

### Step 2

A stirred solution of 2-((1-(2-((tert-butyldimethylsilyl)oxy)ethyl)-1H-pyrazol-3-yl)methyl)-6-(phenylsulfonyl)phthalazin-1(2H)-one (31 mg, 99% Wt, 1 Eq, 58 μmol) in THF (1 mL) was treated with TBAF (46 mg, 0.18 mL, 1.00 molar, 3 Eq, 0.18 mmol) dropwise. The reaction mixture was stirred at RT for 2 h, diluted with EtOAc (10 mL) and washed with 50% brine (10 mL). The organic layer was collected and the aqueous was extracted with EtOAc (2×10 mL). The combined organic extracts were washed with brine (10 mL), dried (MgSO₄) and concentrated in vacuo to afford the crude product as a yellow gum. The crude product was purified by chromatography on RP Flash C18 (12 g cartridge, 10-80% MeCN/10 mM NH₃OH (aq.)) to afford the title compound (13.5 mg) as a white solid. ¹H NMR (400 MHz, CDCl3) δ 8.54 (d, J=8.4 Hz, 1H), 8.32 (d, J=1.7 Hz, 1H), 8.24 (s, 1H), 8.18 (dd, J=8.4, 1.8 Hz, 1H), 8.01-7.95 (m, 2H), 7.65-7.59 (m, 1H), 7.58-7.51 (m, 2H), 7.35 (d, J=2.3 Hz, 1H), 6.28 (d, J=2.3 Hz, 1H), 5.42 (s, 2H), 4.23-4.17 (m, 2H), 3.94 (dd, J=5.6, 4.0 Hz, 2H). (1 exchangeable protons not observed in CDCl3). MS (ES⁺): 411 (M+H)+.

### Example 11—2-((1-ethyl-1H-pyrazol-3-yl)methyl)-6-(phenylsulfonyl)phthalazin-1(2H)-one

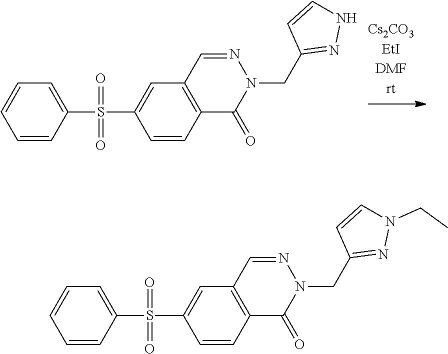 [SMILES: O=c1c2ccc(S(=O)(=O)c3ccccc3)cc2cnn1Cc1cc[nH]n1 ; CCn1ccc(Cn2ncc3cc(S(=O)(=O)c4ccccc4)ccc3c2=O)n1]

Iodoethane (47 mg, 24 μL, 2.0 Eq, 0.30 mmol) was added to a stirred suspension of 2-((1H-pyrazol-3-yl)methyl)-6-(phenylsulfonyl)phthalazin-1(2H)-one (Example 68, 0.055 g, 1 Eq, 0.15 mmol) and cesium carbonate (98 mg, 2.0 Eq, 0.30 mmol) in DMF (2.0 mL) at −10° C. The resulting mixture was stirred overnight while warming up to RT. The reaction mixture was concentrated under reduced pressure, diluted in DCM (10 mL), and washed with water (2×10 mL). The organic layer was dried over MgSO₄ and concentrated under reduced pressure to afford a crude product. The crude product was purified by chromatography on silica gel to afford the title compound (5.3 mg) as a white solid. 2-((1-ethyl-1H-pyrazol-3-yl)methyl)-6-(phenylsulfonyl)phthalazin-1(2H)-one: ¹H NMR (DMSO-d6) δ: 8.68 (d, J=1.9 Hz, 1H), 8.60 (s, 1H), 8.43 (d, J=8.5 Hz, 1H), 8.31 (dd, J=8.5, 1.9 Hz, 1H), 8.06-8.00 (m, 2H), 7.78-7.71 (m, 1H), 7.70-7.63 (m, 2H), 7.61 (d, J=2.2 Hz, 1H), 6.08 (d, J=2.2 Hz, 1H), 5.25 (s, 2H), 4.04 (q, J=7.3, 7.3, 7.3 Hz, 2H), 1.31 (t, J=7.3, 7.3 Hz, 3H). MS (ES⁺): 395 (M+H)⁺.

### Example 12—2-((4-methyl-1H-pyrazol-3-yl)methyl)-6-(phenylsulfonyl)phthalazin-1(2H)-one

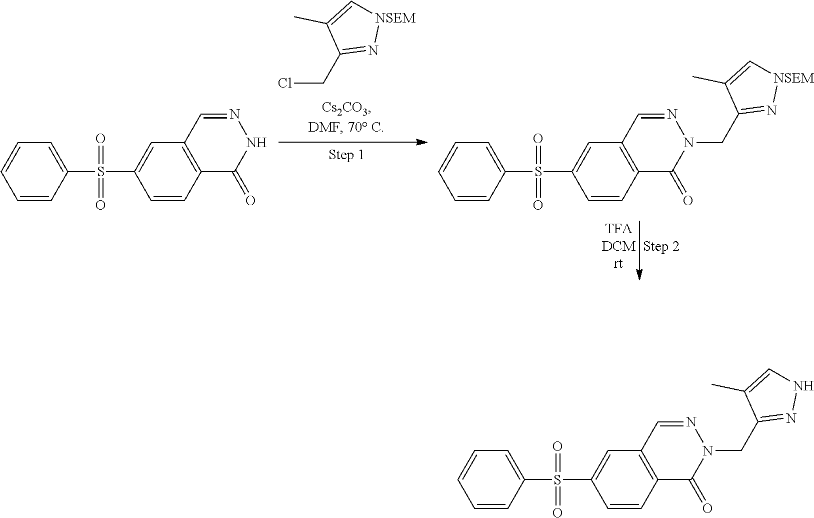 [SMILES: O=c1[nH]ncc2cc(S(=O)(=O)c3ccccc3)ccc12 ; Cc1cn(COCC[Si](C)(C)C)nc1CCl ; Cc1cn(COCC[Si](C)(C)C)nc1Cn1ncc2cc(S(=O)(=O)c3ccccc3)ccc2c1=O ; Cc1c[nH]nc1Cn1ncc2cc(S(=O)(=O)c3ccccc3)ccc2c1=O]

### Step 1

Cesium carbonate (546 mg, 1.6 Eq, 1.68 mmol) was added to a stirred solution of 6-(phenylsulfonyl)phthalazin-1(2H)-one (Intermediate 2, 300 mg, 1 Eq, 1.05 mmol) in dry DMF (6 mL). The reaction mixture was stirred for 5 minutes, then 3-(chloromethyl)-4-methyl-1-((2-(trimethylsilyl)ethoxy)methyl)-1H-pyrazole (Intermediate 10, 354 mg, 85% Wt, 1.1 Eq, 1.15 mmol) was added. The resulting mixture was stirred at 70° C. for 3 h. After cooling to RT, the reaction mixture was diluted with water (20 mL) and extracted with EtOAc (2×20 mL). The organic phase was collected, dried (MgSO₄) and evaporated under reduced pressure. The crude product was purified by chromatography on silica gel to afford 2-((4-methyl-1-((2-(trimethylsilyl)ethoxy)methyl)-1H-pyrazol-3-yl)methyl)-6-(phenylsulfonyl)phthalazin-1(2H)-one (0.57 g, 0.89 mmol, 85%) as a sticky orange oil. MS (ES⁺): 511 (M+H)⁺.

### Step 2

TFA (1.0 g, 0.69 mL, 10 Eq, 8.9 mmol) was added dropwise to a suspension of 2-((4-methyl-1-((2-(trimethylsilyl)ethoxy)methyl)-1H-pyrazol-3-yl)methyl)-6-(phenylsulfonyl)phthalazin-1(2H)-one (0.57 g, 80% Wt, 1 Eq, 0.89 mmol) in DCM (10 mL) at 0° C. The reaction mixture was allowed to warm to RT and stirred for 22 h. The reaction mixture was quenched with sat. aq. ammonium chloride (30 mL) and extracted with DCM (2×20 mL). The organic phase was collected, dried and evaporated under reduced pressure. The crude product was purified by chromatography on silica gel to afford 2-((4-methyl-1H-pyrazol-3-yl)methyl)-6-(phenylsulfonyl)phthalazin-1(2H)-one (0.19 g) as a pale yellow solid. ¹H NMR (400 MHz, DMSO-d6) δ 12.39 (s, 1H), 8.67 (d, J=1.8 Hz, 1H), 8.58 (s, 1H), 8.43 (d, J=8.5 Hz, 1H), 8.30 (dd, J=8.4, 1.9 Hz, 1H), 8.11-7.97 (m, 2H), 7.79-7.70 (m, 1H), 7.70-7.62 (m, 2H), 7.35 (s, 1H), 5.27 (s, 2H), 1.95 (s, 3H). MS (ES⁺): 381 (M+H)⁺.

### Example 13—3-((1-oxo-6-(phenylsulfonyl)phthalazin-2(1H)-yl)methyl)thiophene-2-carboxamide

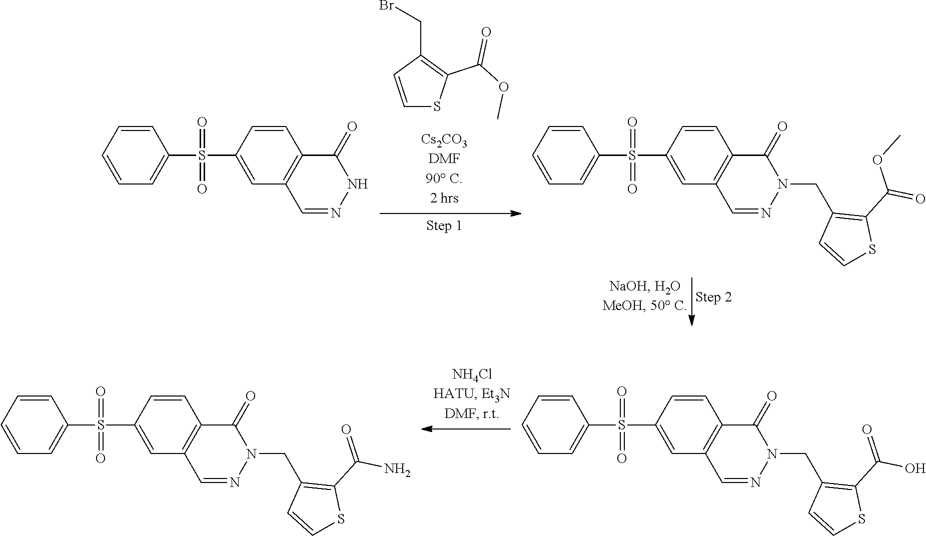 [SMILES: COC(=O)c1sccc1CBr ; O=c1[nH]ncc2cc(S(=O)(=O)c3ccccc3)ccc12 ; COC(=O)c1sccc1Cn1ncc2cc(S(=O)(=O)c3ccccc3)ccc2c1=O ; NC(=O)c1sccc1Cn1ncc2cc(S(=O)(=O)c3ccccc3)ccc2c1=O ; O=C(O)c1sccc1Cn1ncc2cc(S(=O)(=O)c3ccccc3)ccc2c1=O]

### Step 1

To a solution of 6-(phenylsulfonyl)phthalazin-1(2H)-one (Intermediate 2, 200 mg, 0.70 mmol) and K₂CO₃ (179 mg, 1.4 mmol) in DMF (5 mL) was added methyl 3-(bromomethyl)thiophene-2-carboxylate (246 mg, 1.05 mmol) at room temperature, and the reaction mixture was stirred at 90° C. for 2 h. After LCMS indicated the reaction completed, the reaction mixture was concentrated at 40° C. under reduced pressure. The residue was purified by flash column chromatography to give methyl 3-((1-oxo-6-(phenylsulfonyl)phthalazin-2(1H)-yl)methyl)thiophene-2-carboxylate (200 mg) as yellow solid. MS (ES⁺): 440.8 (M+H)⁺.

### Step 2

A mixture of methyl 3-((1-oxo-6-(phenylsulfonyl)phthalazin-2(1H)-yl)methyl)thiophene-2-carboxylate (154 mg, 0.35 mmol) and NaOH (42 mg, 1.05 mmol) in H₂O (5 mL) and MeOH (5 mL) was stirred at 50° C. overnight. After LCMS indicated the reaction completed, the mixture was quenched with 2 N HCl. The mixture was concentrated at 40° C. to remove organic solvent; the residue was separated and extracted with ethyl acetate (3×10 mL). The separated organics were washed with brine, dried over Na₂SO₄ and filtered. The filtrate was concentrated 40° C. under reduced pressure to give 3-((1-oxo-6-(phenylsulfonyl)phthalazin-2(1H)-yl)methyl)thiophene-2-carboxylic acid (10 Omg, 69%) as yellow oil. MS (ES⁺): 427.0 (M+H)⁺.

### Step 3

A mixture of 3-((1-oxo-6-(phenylsulfonyl)phthalazin-2(1H)-yl)methyl)benzoic acid (60 mg, 0.14 mmol), NH₄Cl (30 mg, 0.56 mmol), HATU (106 mg, 0.28 mmol), and Et₃N (57 mg, 0.56 mmol) in DMF (5 mL) was stirred at RT for 16 h. After LCMS indicated the reaction completed, the residue was purified by prep-HPLC (Basic method (B)) The fractions were concentrated at 42° C. under reduced pressure to remove MeCN, and the residue was lyophilized to give the title compound (48.23 mg) as white solid. ¹H NMR (400 MHz, DMSO-d6) δ: 8.72 (d, J=1.2 Hz, 1H), 8.66 (s, 1H), 8.44 (d, J=8.4 Hz, 1H), 8.33 (dd, J₁=2.0 Hz, J₂=8.4 Hz, 1H), 8.04 (d, J=8.0 Hz, 2H), 7.90-7.81 (m, 1H), 7.77-7.73 (m, 1H), 7.70-7.65 (m, 2H), 7.54 (d, J=2.4 Hz, 2H), 6.79 (d, J=7.2 Hz, 1H), 5.57 (s, 2H). MS (ES⁺): 426.0 (M+H)⁺.

### Example 14—6-methoxy-3-((1-oxo-6-(phenylsulfonyl)phthalazin-2(1H)-yl)methyl)picolinamide

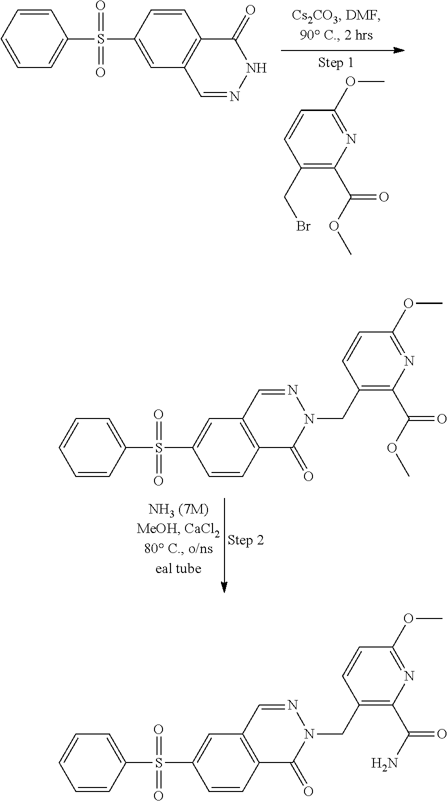 [SMILES: O=c1[nH]ncc2cc(S(=O)(=O)c3ccccc3)ccc12 ; COC(=O)c1nc(OC)ccc1CBr ; COC(=O)c1nc(OC)ccc1Cn1ncc2cc(S(=O)(=O)c3ccccc3)ccc2c1=O ; COc1ccc(Cn2ncc3cc(S(=O)(=O)c4ccccc4)ccc3c2=O)c(C(N)=O)n1]

### Step 1

To a solution of 6-(phenylsulfonyl)phthalazin-1(2H)-one (Intermediate 2, 200 mg, 0.70 mmol) and K₂CO₃ (179 mg, 1.4 mmol) in DMF (5 mL) was added methyl 3-(bromomethyl)-6-methoxypicolinate (Intermediate 19, 200 mg, 0.77 mmol) at RT, and the reaction mixture was stirred at 90° C. for 2 h. After LCMS indicated the reaction completed, the reaction mixture was concentrated at 40° C. under reduced pressure. The residue was purified by flash column chromatography to give methyl 6-methoxy-3-((1-oxo-6-(phenylsulfonyl)phthalazin-2(1H)-yl)methyl)picolinate (180 mg) as yellow solid. MS (ES⁺): 466.0 (M+H)⁺.

### Step 2

In a sealed-tube of methyl 6-methoxy-3-((1-oxo-6-(phenylsulfonyl)phthalazin-2(1H)-yl)methyl)picolinate (180 mg, 0.39 mmol) in MeOH (2 mL) and CaCl₂ (43 mg, 0.39 mmol), NH₃ (7M in MeOH) (8 mL) was added and the reaction mixture was stirred at 80° C. overnight. After LCMS indicated the reaction completed, the reaction mixture was concentrated at 40° C. under reduced pressure. The residue was purified by prep-HPLC (Column: Waters X-SELECT C18 OBD 10 μm 19*250 mm; Flow Rate: 20 mL/min; solvent system: MeCN/(10 mmol/L NH₄HCO₃/water) gradient: MeCN: 40%-95%; collection wavelength: 214 nm). The fractions were concentrated at 42° C. under reduced pressure to remove MeCN, and the residue was lyophilized to give the title compound (88.45 mg) as white solid. ¹H NMR (400 MHz, DMSO-d6) δ: 8.72 (d, J=1.6 Hz, 1H), 8.64 (s, 1H), 8.43 (d, J=8.4 Hz, 1H), 8.33 (dd, J=1.6 Hz, J₂=8.4 Hz, 1H), 8.06-8.03 (m, 3H), 7.77-7.73 (m, 1H), 7.70-7.65 (m, 3H), 7.34 (d, J=8.8 Hz, 1H), 6.83 (d, J=8.4 Hz, 1H), 5.72 (s, 2H), 3.92 (s, 3H). MS (ES⁺): 451.0 (M+H)⁺.

### Example 15—3-((1-oxo-6-(phenylsulfonyl)phthalazin-2(1H)-yl)methyl)-1H-pyrazole-4-carboxamide

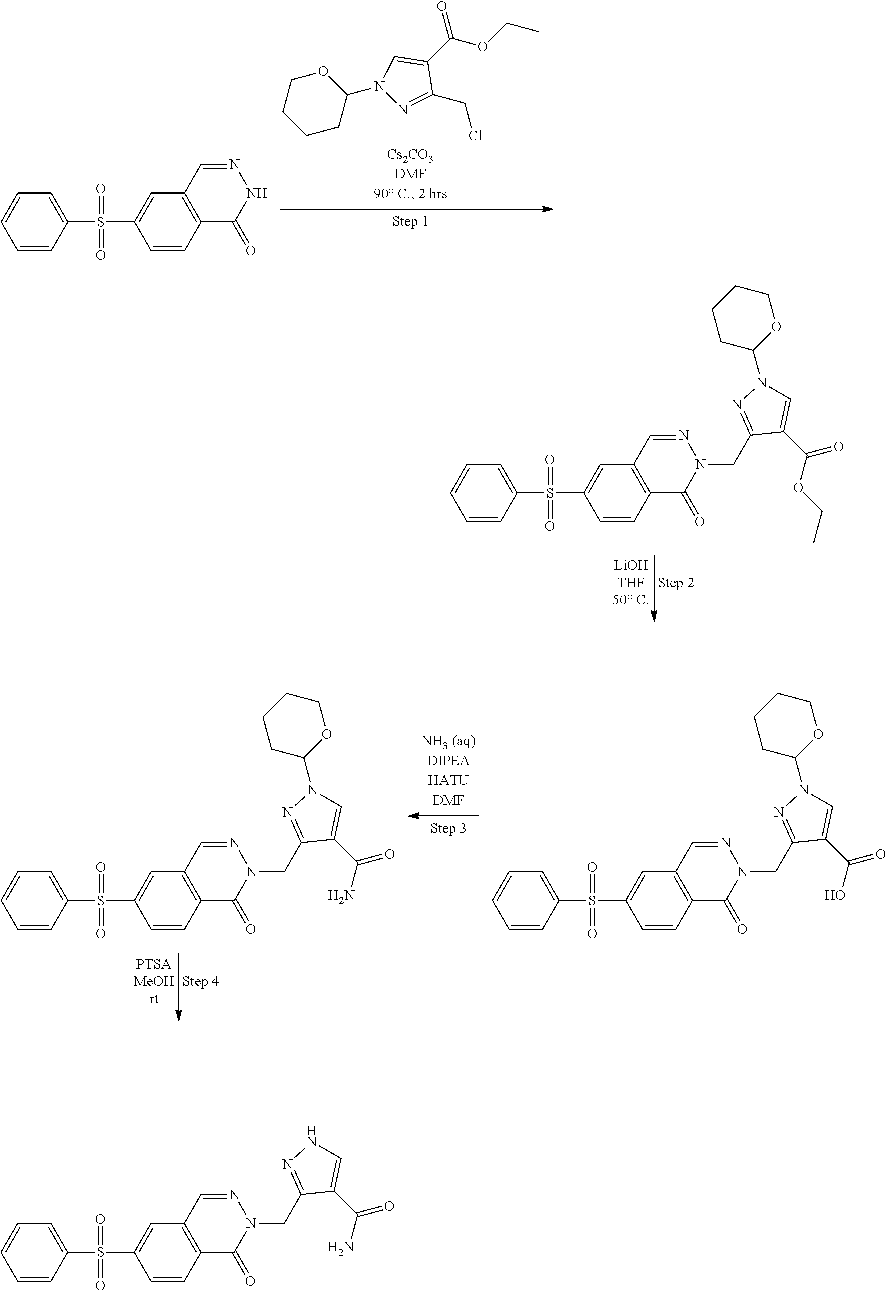 [SMILES: CCOC(=O)c1cn(C2CCCCO2)nc1CCl ; O=c1[nH]ncc2cc(S(=O)(=O)c3ccccc3)ccc12 ; CCOC(=O)c1cn(C2CCCCO2)nc1Cn1ncc2cc(S(=O)(=O)c3ccccc3)ccc2c1=O ; NC(=O)c1cn(C2CCCCO2)nc1Cn1ncc2cc(S(=O)(=O)c3ccccc3)ccc2c1=O ; O=C(O)c1cn(C2CCCCO2)nc1Cn1ncc2cc(S(=O)(=O)c3ccccc3)ccc2c1=O ; NC(=O)c1c[nH]nc1Cn1ncc2cc(S(=O)(=O)c3ccccc3)ccc2c1=O]

A stirred suspension of 6-(phenylsulfonyl)phthalazin-1(2H)-one (Intermediate 2, 336 mg, 1 Eq, 1.17 mmol) and cesium carbonate (421 mg, 1.1 Eq, 1.29 mmol) in DMF (5 mL) was heated to 80° C. for 2 h and then allowed to cool to RT. A solution of ethyl 3-(chloromethyl)-1-(tetrahydro-2H-pyran-2-yl)-1H-pyrazole-4-carboxylate (Intermediate 8, 356 mg, 90% Wt, 1 Eq, 1.17 mmol) in DMF (2 mL) was added and the reaction mixture was stirred at room temperature for 20 h. The reaction mixture was concentrated in vacuo and the residue was azeotroped with toluene (2 times) to afford the crude product. The crude product was purified by chromatography on silica gel to afford ethyl 3-((1-oxo-6-(phenylsulfonyl)phthalazin-2(1H)-yl)methyl)-1-(tetrahydro-2H-pyran-2-yl)-1H-pyrazole-4-carboxylate (460 mg) as a pale yellow solid. MS (ES⁺): 523 (M+H)⁺.

### Step 2

A mixture of ethyl 3-((1-oxo-6-(phenylsulfonyl)phthalazin-2(1H)-yl)methyl)-1-(tetrahydro-2H-pyran-2-yl)-1H-pyrazole-4-carboxylate (200 mg, 87% Wt, 1 Eq, 333 μmol) and lithium hydroxide (15.9 mg, 2 Eq, 666 μmol) was dissolved in THF (2 mL) and water (1 mL) and then the reaction mixture was stirred at 50° C. for 3 h. The mixture was allowed to stand at RT for 18 h. 0.5 M HCl (aq.) (6 mL) was added and the mixture was extracted with EtOAc (2×10 mL). The combined organic extracts were washed with brine (10 mL), dried (MgSO₄) and concentrated in vacuo. The residue was dissolved in DCM and concentrated in vacuo. The residue was azeotroped with diethyl ether afford 3-((1-oxo-6-(phenylsulfonyl)phthalazin-2(1H)-yl)methyl)-1-(tetrahydro-2H-pyran-2-yl)-1H-pyrazole-4-carboxylic acid (196 mg) as a pale yellow solid. MS (ES⁺): 517 (M+Na)⁺.

### Step 3

A stirred solution of 3-((1-oxo-6-(phenylsulfonyl)phthalazin-2(1H)-yl)methyl)-1-(tetrahydro-2H-pyran-2-yl)-1H-pyrazole-4-carboxylic acid (196 mg, 80% Wt, 1 Eq, 317 μmol), DIPEA (52 mg, 70 μL, 1.3 Eq, 0.40 mmol) and ammonia (0.5 M in THF) (55.3 mg, 6.50 mL, 0.50 molar, 10.2 Eq, 3.25 mmol) was treated with a solution of HATU (127 mg, 1.05 Eq, 333 μmol) in DMF (2 mL). The reaction mixture was stirred at RT for 2 h. Further ammonia (0.5 M in THF) (55.3 mg, 6.50 mL, 0.50 molar, 10.2 Eq, 3.25 mmol) was added and the mixture stirred for 18 h. Added ammonium hydroxide (176 mg, 200 μL, 26% Wt, 4.12 Eq, 1.31 mmol) stirred for 3 h. The reaction mixture was diluted with EtOAc (10 mL) and washed with sat. aq. NaHCO₃ (10 mL). The organic layer was collected and the aqueous was extracted with EtOAc (2×10 mL). The combined organic extracts were washed with brine (10 mL), dried (MgSO₄) and concentrated in vacuo to afford the crude product. The crude product was purified by chromatography on silica gel to afford 3-((1-oxo-6-(phenylsulfonyl)phthalazin-2(1H)-yl)methyl)-1-(tetrahydro-2H-pyran-2-yl)-1H-pyrazole-4-carboxamide (165 mg) as a white solid. ¹H NMR (400 MHz, DMSO-d6) δ 8.70 (d, J=1.8 Hz, 1H), 8.58 (s, 1H), 8.43 (d, J=8.4 Hz, 1H), 8.40 (s, 1H), 8.31 (dd, J=8.5, 1.9 Hz, 1H), 8.08-8.01 (m, 2H), 7.80-7.73 (m, 1H), 7.67 (t, J=7.6 Hz, 2H), 7.54 (s, 1H), 7.04 (s, 1H), 5.52 (s, 2H), 5.22 (dd, J=9.6, 2.5 Hz, 1H), 3.84 (d, J=11.8 Hz, 1H), 3.59-3.48 (m, 1H), 1.85-1.68 (m, 3H), 1.59-1.51 (m, 1H), 1.45 (d, J=7.5 Hz, 2H). MS (ES⁺): 494 (M+H)⁺.

### Step 4

A solution of 3-((1-oxo-6-(phenylsulfonyl)phthalazin-2(1H)-yl)methyl)-1-(tetrahydro-2H-pyran-2-yl)-1H-pyrazole-4-carboxamide (70.0 mg, 88% Wt, 1 Eq, 125 μmol) in MeOH (1 mL) was treated with p-toluenesulfonic acid monohydrate (23.7 mg, 1 Eq, 125 μmol). The reaction mixture was stirred at RT for 24 h. The reaction mixture was concentrated in vacuo to afford the crude product. The crude product was purified by chromatography on silica gel to afford the title compound (5.6 mg) as a white solid. ¹H NMR (400 MHz, DMSO-d6) δ 8.70 (d, J=1.8 Hz, 1H), 8.58 (s, 1H), 8.43 (d, J=8.4 Hz, 1H), 8.31 (dd, J=8.4, 1.9 Hz, 1H), 8.11 (s, 1H), 8.07-8.01 (m, 2H), 7.79-7.72 (m, 1H), 7.67 (dd, J=8.3, 6.8 Hz, 2H), 7.52 (s, 1H), 6.98 (s, 1H), 5.55 (s, 2H). (1 exchangeable proton not observed in DMSO). MS (ES⁺): 410.1 (M+H)⁺.

### Example 16—2-((1-(2-hydroxyethyl)-4-methyl-1H-pyrazol-3-yl)methyl)-6-(phenylsulfonyl)phthalazin-1(2H)-one

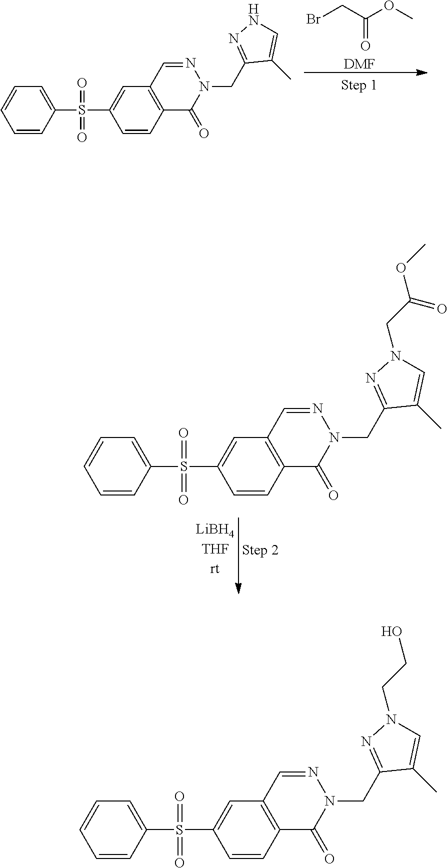

### Step 1

A stirred suspension of 2-((4-methyl-1H-pyrazol-3-yl)methyl)-6-(phenylsulfonyl)phthalazin-1(2H)-one (Example 12, 40 mg, 95% Wt, 1 Eq, 0.10 mmol) and cesium carbonate (65 mg, 2 Eq, 0.20 mmol) in DMF (3 mL) was treated with methyl bromoacetate (23 mg, 14 μL, 1.5 Eq, 0.15 mmol). The reaction mixture was stirred for 13 h at 60° C., then allowed to cool to RT and absorbed on silica. The crude product was purified by chromatography on silica gel to afford methyl 2-(4-methyl-3-((1-oxo-6-(phenylsulfonyl)phthalazin-2(1H)-yl)methyl)-1H-pyrazol-1-yl)acetate (35 mg) as a pale yellow solid. MS (ES⁺): 453 (M+H)⁺.

### Step 2

Lithium borohydride (2.9 mg, 66 μL, 2.00 molar, 2 Eq, 0.13 mmol) was added dropwise to a stirred solution of methyl 2-(4-methyl-3-((1-oxo-6-(phenylsulfonyl)phthalazin-2(1H)-yl)methyl)-1H-pyrazol-1-yl)acetate (30 mg, 1 Eq, 66 μmol) in dry THF (3.0 mL). The reaction mixture was stirred for 2 h at RT. The reaction mixture was diluted with water (25 mL) and transferred into a separating funnel. The layer was extracted with EtOAc (2×25 mL). The combined organic layers were collected, dried (MgSO₄), filtered and concentrated in vacuo. The crude product was dissolved in DMSO (1 mL), filtered and purified by reversed phase preparative HPLC (Basic method (B)). The relevant fractions were evaporated in a Genevac., yielding the title compound (7 mg) as a clear white solid. ¹H NMR (400 MHz, DMSO-d6) δ 8.67 (d, J=1.9 Hz, 1H), 8.58 (s, 1H), 8.43 (d, J=8.4 Hz, 1H), 8.30 (dd, J=8.5, 1.9 Hz, 1H), 8.03 (dd, J=7.3, 1.7 Hz, 2H), 7.79-7.71 (m, 1H), 7.66 (dd, J=8.6, 7.0 Hz, 2H), 7.39 (s, 1H), 5.23 (s, 2H), 4.80 (t, J=5.3 Hz, 1H), 3.97 (t, J=5.7 Hz, 2H), 3.62 (q, J=5.5 Hz, 2H), 1.93 (s, 3H). MS (ES⁺): 425 (M+H)⁺.

### Example 17—3-((6-((4-methoxyphenyl)sulfonyl)-1-oxophthalazin-2(1H)-yl)methyl)-N-methylfuran-2-carboxamide

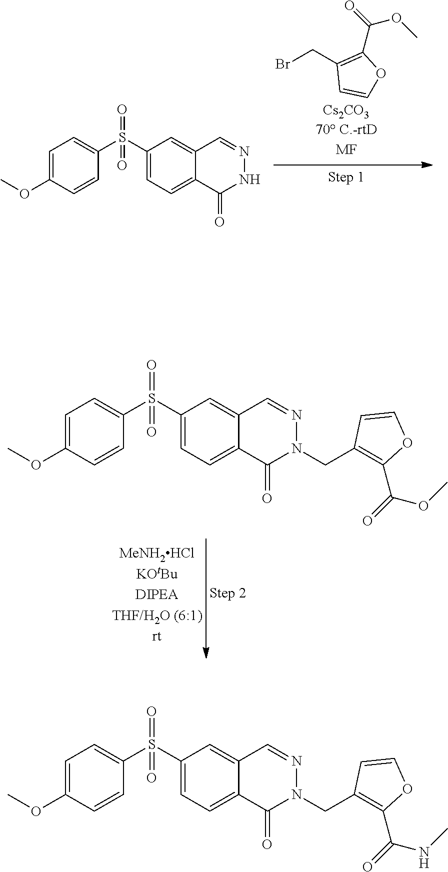

### Step 1

To a stirred solution of 6-((4-methoxyphenyl)sulfonyl)phthalazin-1(2H)-one (Intermediate 1, 0.10 g, 66% Wt, 1 Eq, 0.21 mmol) in dry DMF (8.0 mL) was added cesium carbonate (0.20 g, 3 Eq, 0.63 mmol). The reaction mixture was stirred for 5 minutes, then methyl 3-(bromomethyl)furan-2-carboxylate (Intermediate 18, 91 mg, 2 Eq, 0.42 mmol) was added in a single portion. The resulting mixture was stirred at 70° C. for 3 h, then it was cooled to RT and diluted with water (10 mL) and extracted with DCM (2×10 mL). The organic phase was collected, dried and evaporated under reduced pressure. The crude product was purified by chromatography on silica gel to afford methyl 3-((6-((4-methoxyphenyl)sulfonyl)-1-oxophthalazin-2(1H)-yl)methyl)furan-2-carboxylate (75 mg) as a clear white solid. ¹H NMR (400 MHz, DMSO-d6) δ 8.65 (d, J=1.8 Hz, 1H), 8.62 (s, 1H), 8.42 (d, J=8.4 Hz, 1H), 8.29 (dd, J=8.4, 1.8 Hz, 1H), 7.99-7.93 (m, 2H), 7.82 (d, J=1.8 Hz, 1H), 7.20-7.14 (m, 2H), 6.44 (d, J=1.8 Hz, 1H), 5.50 (s, 2H), 3.85 (s, 3H), 3.83 (s, 3H). MS (ES⁺): 455.3 (M+H)⁺.

### Step 2

Potassium tert-butoxide (89 mg, 6 Eq, 0.79 mmol) was added to a stirred solution of methyl 3-((6-((4-methoxyphenyl)sulfonyl)-1-oxophthalazin-2(1H)-yl)methyl)furan-2-carboxylate (60 mg, 1 Eq, 0.13 mmol) in THF/Water 6:1 (3.5 mL). Methylamine hydrochloride (45 mg, 5 Eq, 0.66 mmol) and DIPEA (85 mg, 0.11 mL, 5 Eq, 0.66 mmol) were added sequentially and the reaction mixture was stirred for 48 h at RT. The reaction was diluted with DCM (5 mL) and washed with water (4 mL), then brine (4 mL). The organic phase was collected, dried and evaporated under reduced pressure. The crude product was purified by chromatography on silica gel to afford the title compound (33 mg) as a pale yellow solid. ¹H NMR (400 MHz, DMSO-d6) δ 8.65 (d, J=1.8 Hz, 1H), 8.60 (s, 1H), 8.42 (d, J=8.4 Hz, 1H), 8.31 (d, J=4.9 Hz, 1H), 8.28 (dd, J=8.4, 1.8 Hz, 1H), 7.99-7.93 (m, 2H), 7.66 (d, J=1.8 Hz, 1H), 7.20-7.14 (m, 2H), 6.33 (d, J=1.8 Hz, 1H), 5.55 (s, 2H), 3.83 (s, 3H), 2.75 (d, J=4.6 Hz, 3H). MS (ES⁺): 454.3 (M+H)⁺.

### Example 18—3-((1-oxo-6-(phenylsulfonyl)phthalazin-2(1H)-yl)methyl)-1H-pyrazole-4-carbonitrile

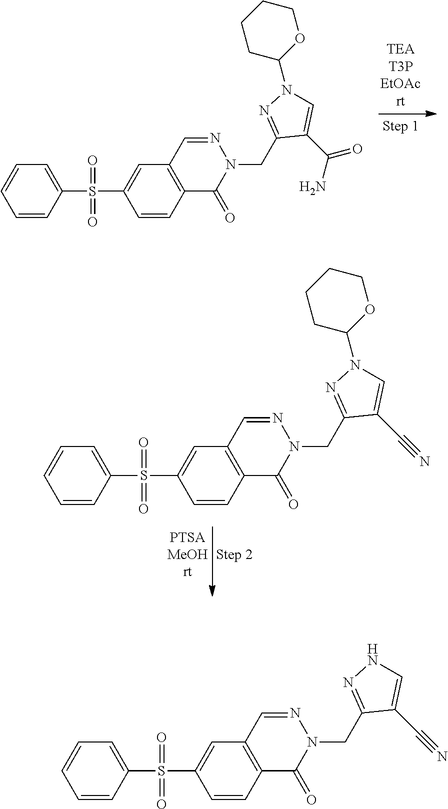 [SMILES: NC(=O)c1cn(C2CCCCO2)nc1Cn1ncc2cc(S(=O)(=O)c3ccccc3)ccc2c1=O ; N#Cc1cn(C2CCCCO2)nc1Cn1ncc2cc(S(=O)(=O)c3ccccc3)ccc2c1=O ; N#Cc1c[nH]nc1Cn1ncc2cc(S(=O)(=O)c3ccccc3)ccc2c1=O]

### Step 1

A solution of 3-((1-oxo-6-(phenylsulfonyl)phthalazin-2(1H)-yl)methyl)-1-(tetrahydro-2H-pyran-2-yl)-1H-pyrazole-4-carboxamide (Example 15 intermediate, 44 mg, 99% Wt, 1 Eq, 88 μmol) and triethylamine (18 mg, 25 μL, 2.0 Eq, 0.18 mmol) in EtOAc (2 mL) was treated with T3P (50 wt % in EtOAc) (0.11 g, 0.11 mL, 50% Wt, 2.0 Eq, 0.18 mmol). The reaction mixture was stirred at 50° C. for 4 h and then allowed to cool to RT and stirred for 18 h. The reaction mixture was concentrated in vacuo to afford the crude product. The crude product was purified by chromatography on silica gel to afford 3-((1-oxo-6-(phenylsulfonyl)phthalazin-2(1H)-yl)methyl)-1-(tetrahydro-2H-pyran-2-yl)-1H-pyrazole-4-carbonitrile (36 mg) as a colourless gum. MS (ES⁺): 498 (M+Na)⁺.

### Step 2

A solution of p-toluenesulfonic acid monohydrate (15 mg, 1.1 Eq, 79 μmol) in MeOH (1 mL) was added to 3-((1-oxo-6-(phenylsulfonyl)phthalazin-2(1H)-yl)methyl)-1-(tetrahydro-2H-pyran-2-yl)-1H-pyrazole-4-carbonitrile (36 mg, 98% Wt, 1 Eq, 74 μmol). The reaction mixture was stirred at RT for 18 h, diluted with DCM (5 mL) and washed with sat. aq. NaHCO₃ (5 mL). The organic layer was collected and the aqueous was extracted with DCM (5 mL). The combined organic extracts were dried and concentrated in vacuo to afford the crude product. The crude product was dissolved in DMSO (1 mL), filtered and purified by reversed phase preparative HPLC (Basic method (B)). The clean fractions were evaporated in a Genevac to afford the title compound (15.3 mg) as a white solid. ¹H NMR (400 MHz, DMSO-d6) b 13.67 (s, 1H), 8.71 (d, J=1.8 Hz, 1H), 8.64 (s, 1H), 8.44 (d, J=8.4 Hz, 1H), 8.39 (br s, 1H), 8.33 (dd, J=8.4, 1.9 Hz, 1H), 8.07-7.99 (m, 2H), 7.79-7.71 (m, 1H), 7.71-7.63 (m, 2H), 5.45 (s, 2H). MS (ES⁺): 390 (M+H)⁺.

### Example 19—2-(2-hydroxy-1-phenylethyl)-6-(phenylsulfonyl)phthalazin-1(2H)-one

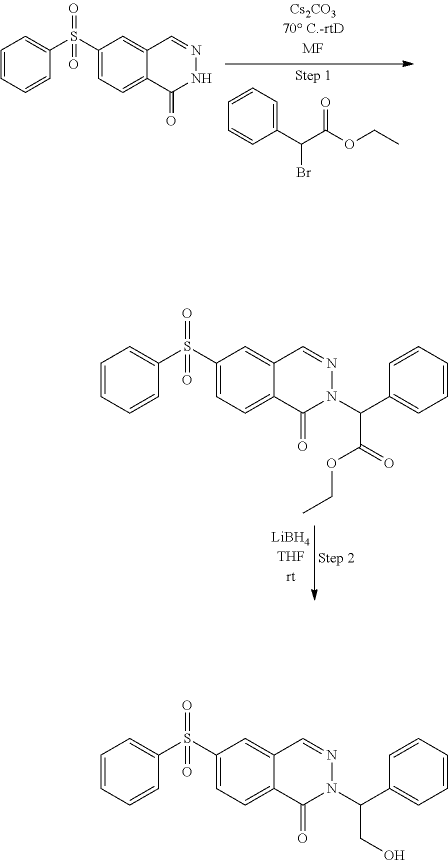

### Step 1

A mixture of 6-(phenylsulfonyl)phthalazin-1(2H)-one (Intermediate 2, 100 mg, 1 Eq, 349 μmol) and cesium carbonate (228 mg, 2 Eq, 699 μmol) in DMF (2 mL) was stirred at 70° C. for 45 minutes. Ethyl 2-bromo-2-phenylacetate (102 mg, 1.2 Eq, 419 μmol) was added and the mixture was allowed to cool to RT and stirred overnight. Water and DCM were added and the layers separated. The organic layer was washed with brine, then absorbed on silica. The crude product was purified by chromatography on silica gel to afford ethyl 2-(1-oxo-6-(phenylsulfonyl)phthalazin-2(1H)-yl)-2-phenylacetate (123 mg) as a white glass. MS (ES⁺): 449.0 (M+H)⁺.

### Step 2

Lithium borohydride (2M in THF) (3.5 mg, 80 μL, 2 molar, 1.2 Eq, 0.16 mmol) was added to a solution of ethyl 2-(1-oxo-6-(phenylsulfonyl)phthalazin-2(1H)-yl)-2-phenylacetate (60 mg, 1 Eq, 0.13 mmol) in THF (0.5 mL) and the mixture was stirred for 4.5 h. 1N HCl and DCM were added and the layers separated. The organic layer was washed with brine, then absorbed on silica. The crude product was purified by chromatography on silica gel to afford the title compound (15 mg) as a white solid, ¹H NMR (DMSO-d6) δ: 8.73-8.67 (m, 2H), 8.43 (d, 1H), 8.30 (dd, 1H), 8.05-7.98 (m, 2H), 7.77-7.71 (m, 1H), 7.70-7.62 (m, 2H), 7.38-7.23 (m, 5H), 6.15 (dd, 1H), 4.96 (t, 1H), 4.26 (ddd, 1H), 3.96-3.87 (m, 1H). MS (ES⁺): 407.0 (M+H)⁺.

### Example 20—2-(2-(hydroxymethyl)benzyl)-6-(phenylsulfonyl)phthalazin-1(2H)-one

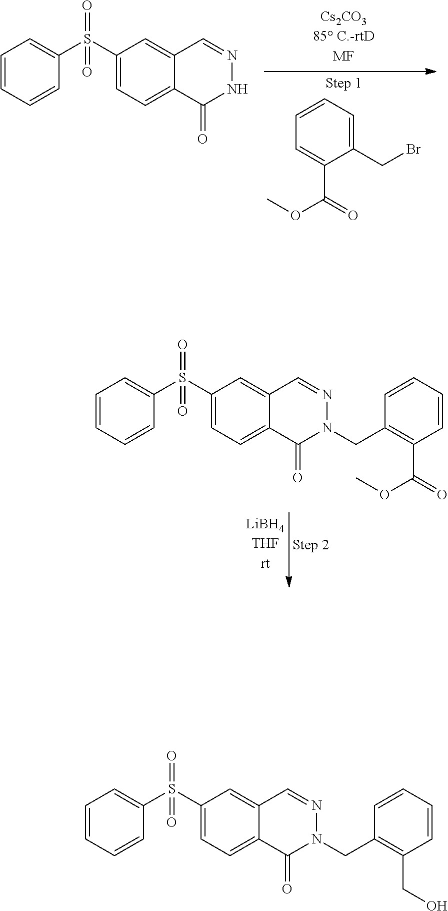 [SMILES: O=c1[nH]ncc2cc(S(=O)(=O)c3ccccc3)ccc12 ; COC(=O)c1ccccc1CBr ; COC(=O)c1ccccc1Cn1ncc2cc(S(=O)(=O)c3ccccc3)ccc2c1=O ; O=c1c2ccc(S(=O)(=O)c3ccccc3)cc2cnn1Cc1ccccc1CO]

### Step 1

A suspension of 6-(phenylsulfonyl)phthalazin-1(2H)-one (Intermediate 2, 100 mg, 1 Eq, 349 μmol) and cesium carbonate (228 mg, 2.0 Eq, 699 μmol) in DMF (2 mL) was stirred at 85° C. for 2 h and then allowed to cool to RT. Methyl 2-(bromomethyl)benzoate (88.0 mg, 1.1 Eq, 384 μmol) was added and the reaction mixture was stirred at RT for 18 h. The reaction mixture was diluted with water (10 mL) and the resultant precipitate was collected by filtration, washing with water. The solid was triturated with diethyl ether, filtered, washed with diethyl ether and dried to afford methyl 2-((1-oxo-6-(phenylsulfonyl)phthalazin-2(1H)-yl)methyl)benzoate (115 mg) as a yellow solid. ¹H NMR (400 MHz, DMSO-d6) δ 8.73 (d, J=1.8 Hz, 1H), 8.66 (s, 1H), 8.44 (d, J=8.4 Hz, 1H), 8.33 (dd, J=8.5, 1.8 Hz, 1H), 8.09-8.01 (m, 2H), 7.91 (dd, J=7.6, 1.6 Hz, 1H), 7.80-7.71 (m, 1H), 7.71-7.63 (m, 2H), 7.46 (td, J=7.6, 1.7 Hz, 1H), 7.40 (td, J=7.5, 1.4 Hz, 1H), 7.03 (dd, J=7.7, 1.4 Hz, 1H), 5.66 (s, 2H), 3.86 (s, 3H). MS (ES⁺): 435.1 (M+H)⁺.

### Step 2

A solution of methyl 2-((1-oxo-6-(phenylsulfonyl)phthalazin-2(1H)-yl)methyl)benzoate (40 mg, 99% Wt, 1 Eq, 91 μmol) in THF (0.5 mL) was treated with lithium borohydride (2.4 mg, 55 μL, 2.00 molar, 1.2 Eq, 0.11 mmol) dropwise. The reaction mixture was stirred at RT for 4 h and then quenched with sat. aq. NaHCO₃ (10 mL). The mixture was extracted with EtOAc (2×10 mL) and the combined organic extracts were washed with brine (10 mL), dried (MgSO₄) and concentrated in vacuo. The residue was dissolved in THF (1 mL), treated with lithium borohydride (100 μL, 2.00 molar, 2.2 eq, 0.2 mmol) and stirred for 2 h. The reaction mixture was quenched with sat. aq. NaHCO₃ (10 mL). The mixture was extracted with EtOAc (2×10 mL) and the combined organic extracts were washed with brine (10 mL), dried (MgSO₄) and concentrated in vacuo to afford the crude product. The crude product was purified by chromatography on RP Flash C18 (12 g cartridge, 10-70% MeCN/10 mM ammonium hydroxide_{(aq)}); relevant fractions were combined and solvent evaporated and then upon isolation the material was triturated with diethyl ether to afford the title compound (5.9 mg) as a white solid. ¹H NMR (400 MHz, DMSO-d6) δ 8.71 (d, J=1.8 Hz, 1H), 8.64 (s, 1H), 8.44 (d, J=8.4 Hz, 1H), 8.32 (dd, J=8.4, 1.9 Hz, 1H), 8.07-8.00 (m, 2H), 7.78-7.72 (m, 1H), 7.70-7.64 (m, 2H), 7.43-7.38 (m, 1H), 7.24 (td, J=7.4, 1.4 Hz, 1H), 7.14 (td, J=7.5, 1.5 Hz, 1H), 7.00 (dd, J=7.8, 1.3 Hz, 1H), 5.39 (s, 2H), 5.16 (t, J=5.4 Hz, 1H), 4.65 (d, J=5.4 Hz, 2H). MS (ES⁺): 407.1 (M+H)⁺.

### Example 21—2-((6-(2-hydroxyethoxy)pyridin-3-yl)methyl)-6-(phenylsulfonyl)phthalazin-1(2H)-one

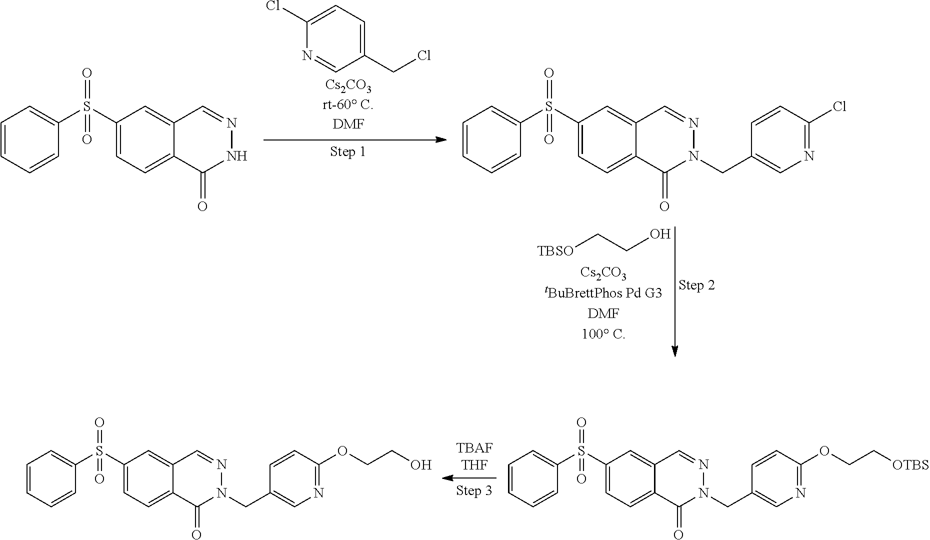

### Step 1

To a stirred solution of 6-(phenylsulfonyl)phthalazin-1(2H)-one (Intermediate 2, 0.30 g, 1 Eq, 1.0 mmol) in dry DMF (10 mL) was added cesium carbonate (0.68 g, 2.0 Eq, 2.1 mmol). The reaction mixture was stirred for 5 minutes, then 2-chloro-5-(chloromethyl)pyridine (0.17 g, 1.0 Eq, 1.0 mmol) was added. The resulting mixture was stirred at RT for 2 h and subsequently heated to 60° C. for a further hour. The mixture was allowed to cool to RT, hence it was poured in an ice/water mixture. The so-obtained precipitate was filtered off and washed with copious amounts of water, giving 2-((6-chloropyridin-3-yl)methyl)-6-(phenylsulfonyl)phthalazin-1(2H)-one (0.42 g) as a brown solid. ¹H NMR (400 MHz, DMSO-d6) δ 8.70 (d, J=1.8 Hz, 1H), 8.64 (s, 1H), 8.45-8.39 (m, 2H), 8.32 (dd, J=8.4, 1.8 Hz, 1H), 8.05-8.01 (m, 2H), 7.79 (dd, J=8.3, 2.5 Hz, 1H), 7.77-7.71 (m, 1H), 7.70-7.63 (m, 2H), 7.46 (d, J=8.3 Hz, 1H), 5.36 (s, 2H). MS (ES⁺): 412.0/414.1 (M+H)⁺.

### Step 2

2-((6-chloropyridin-3-yl)methyl)-6-(phenylsulfonyl)phthalazin-1(2H)-one (0.20 g, 1 Eq, 0.49 mmol) was suspended in DMF (6 mL) at RT. To this mixture was added 2-((tert-butyldimethylsilyl)oxy)ethan-1-ol (0.17 g, 0.19 mL, 2.0 Eq, 0.97 mmol), cesium carbonate (0.40 g, 2.5 Eq, 1.2 mmol), and tBuBrettPhos Pd G3 (41 mg, 0.1 Eq, 49 μmol). The reaction mixture was stirred at 100° C. for 3 h. The reaction was cooled to RT, diluted with water (20 mL) and back-extracted with EtOAc (2×20 mL). The organic phase was washed with brine (20 mL), collected, dried (MgSO₄) and evaporated under reduced pressure. The crude product was purified by chromatography on silica gel to afford 2-((6-(2-((tert-butyldimethylsilyl)oxy)ethoxy)pyridin-3-yl)methyl)-6-(phenylsulfonyl)phthalazin-1(2H)-one (0.15 g) as a pale brown solid. ¹H NMR (400 MHz, DMSO-d6) δ 8.68 (d, J=1.8 Hz, 1H), 8.62 (s, 1H), 8.42 (d, J=8.4 Hz, 1H), 8.30 (dd, J=8.5, 1.9 Hz, 1H), 8.16 (d, J=2.4 Hz, 1H), 8.05-7.99 (m, 2H), 7.77-7.71 (m, 1H), 7.70-7.61 (m, 3H), 6.73 (d, J=8.5 Hz, 1H), 5.26 (s, 2H), 4.26 (dd, J=5.8, 4.1 Hz, 2H), 3.86 (dd, J=5.8, 4.1 Hz, 2H), 0.79 (s, 9H), −0.00 (s, 6H). MS (ES⁺): 552.2 (M+H)⁺.

### Step 3

To a stirred solution of 2-((6-(2-((tert-butyldimethylsilyl)oxy)ethoxy)pyridin-3-yl)methyl)-6-(phenylsulfonyl)phthalazin-1(2H)-one (0.14 g, 1 Eq, 0.25 mmol) in dry THF (5 mL) was added TBAF (0.20 g, 0.76 mL, 1 molar, 3 Eq, 0.76 mmol). The reaction mixture was stirred for 16 h at RT. The reaction was diluted with water (20 mL) and the so-formed precipitate was filtered off and washed with water, yielding the crude product. The crude product was purified by chromatography on silica gel to afford the title compound (81 mg) as a flocculent white solid. ¹H NMR (400 MHz, DMSO-d⁶) δ 8.68 (d, J=1.8 Hz, 1H), 8.62 (s, 1H), 8.43 (d, J=8.5 Hz, 1H), 8.31 (dd, J=8.4, 1.9 Hz, 1H), 8.16 (d, J=2.4 Hz, 1H), 8.05-7.98 (m, 2H), 7.77-7.70 (m, 1H), 7.69-7.63 (m, 3H), 6.75 (d, J=8.5 Hz, 1H), 5.26 (s, 2H), 4.78 (t, J=5.5 Hz, 1H), 4.24-4.19 (m, 2H), 3.66 (q, J=5.4 Hz, 2H). MS (ES⁺): 438.1 (M+H)⁺.

### Example 22—2-((2-(hydroxymethyl)thiophen-3-yl)methyl)-6-(phenylsulfonyl)phthalazin-1(2H)-one

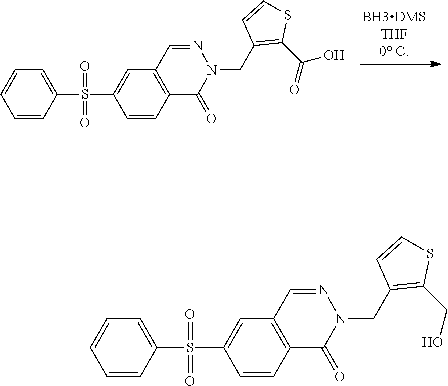 [SMILES: O=C(O)c1sccc1Cn1ncc2cc(S(=O)(=O)c3ccccc3)ccc2c1=O ; O=c1c2ccc(S(=O)(=O)c3ccccc3)cc2cnn1Cc1ccsc1CO]

Borane-methyl sulfide complex (14 mg, 94 μL, 2 molar, 2 Eq, 0.19 mmol) was added dropwise to a solution of 3-((1-oxo-6-(phenylsulfonyl)phthalazin-2(1H)-yl)methyl)thiophene-2-carboxylic acid (Example 13 intermediate, 40 mg, 1 Eq, 94 μmol) in dry THF (3.0 mL) at 0° C. The reaction mixture was stirred for 2 h at 0° C., then allowed to warm to RT and stirred for 40 h. The reaction mixture was diluted with water (25 mL) and transferred into a separating funnel. The layer was extracted with EtOAc (2×25 mL). The combined organic layers were collected, dried over (MgSO₄), filtered and concentrated in vacuo. The crude product was purified by chromatography on silica gel to afford the title compound (30 mg) as a clear white solid. ¹H NMR (400 MHz, DMSO-d6) δ 8.68 (d, J=1.8 Hz, 1H), 8.62 (s, 1H), 8.43 (d, J=8.4 Hz, 1H), 8.30 (dd, J=8.4, 1.8 Hz, 1H), 8.02 (dd, J=7.5, 1.7 Hz, 2H), 7.74 (t, J=7.3 Hz, 1H), 7.66 (dd, J=8.4, 6.8 Hz, 2H), 7.29 (d, J=5.1 Hz, 1H), 6.92 (d, J=5.1 Hz, 1H), 5.42 (t, J=5.5 Hz, 1H), 5.25 (s, 2H), 4.77 (d, J=5.5 Hz, 2H). MS (ES⁺): 395 (M+H)⁺.

### Example 23—3-((1-oxo-6-(phenylsulfonyl)phthalazin-2(1H)-yl)methyl)thiophene-2-carbonitrile

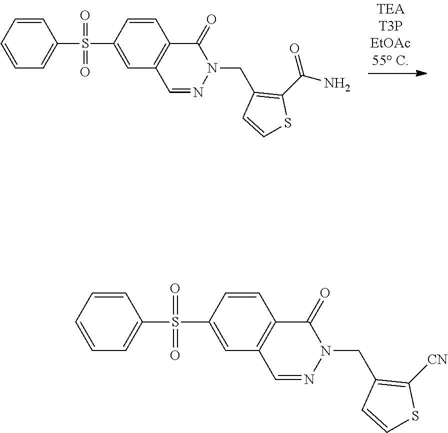 [SMILES: NC(=O)c1sccc1Cn1ncc2cc(S(=O)(=O)c3ccccc3)ccc2c1=O ; N#Cc1sccc1Cn1ncc2cc(S(=O)(=O)c3ccccc3)ccc2c1=O]

A solution of 3-((1-oxo-6-(phenylsulfonyl)phthalazin-2(1H)-yl)methyl)thiophene-2-carboxamide (Example 13, 70 mg, 98% Wt, 1 Eq, 0.16 mmol) and TEA (65 mg, 90 μL, 4.0 Eq, 0.64 mmol) in EtOAc (1 mL) was treated with T3P (50 wt % in EtOAc) (0.21 g, 0.19 mL, 50% Wt, 2.0 Eq, 0.32 mmol) and the mixture was stirred at RT for 18 h, then was heated to 55° C. for 3 h. The reaction mixture was concentrated in vacuo to afford the crude product. The crude product was purified by chromatography on silica gel to afford the title compound (30 mg) as a pale orange solid. ¹H NMR (400 MHz, DMSO-d6) δ 8.71 (d, J=1.8 Hz, 1H), 8.66 (s, 1H), 8.44 (d, J=8.4 Hz, 1H), 8.33 (dd, J=8.5, 1.8 Hz, 1H), 8.04 (dd, J=7.9, 1.6 Hz, 2H), 7.98 (d, J=5.1 Hz, 1H), 7.75 (t, J=7.4 Hz, 1H), 7.67 (dd, J=8.4, 6.9 Hz, 2H), 7.19 (d, J=5.1 Hz, 1H), 5.45 (s, 2H). MS (ES⁺): 408 (M+H)⁺.

### Example 24—2-((1-(2-hydroxyethyl)-1H-pyrazol-3-yl)methyl)-6-((4-methoxyphenyl)sulfonyl)phthalazin-1(2H)-one

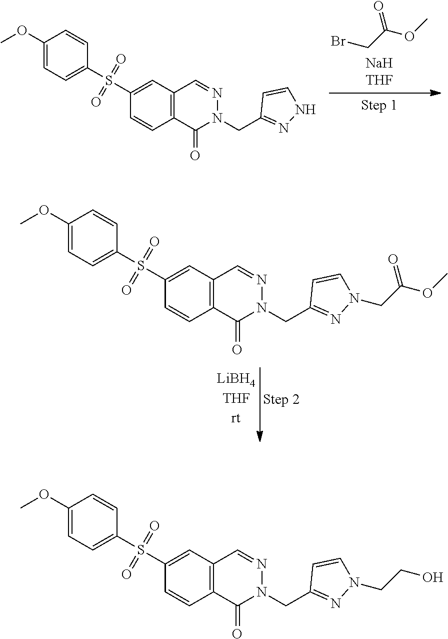

### Step 1

Sodium hydride (2.4 mg, 60% Wt, 1.2 Eq, 61 μmol) was added to a solution of 2-((1H-pyrazol-3-yl)methyl)-6-((4-methoxyphenyl)sulfonyl)phthalazin-1(2H)-one (Example 6, 20 mg, 1 Eq, 50 μmol) in THF (0.25 mL) and the mixture was stirred for 5 minutes under N₂. Methyl 2-bromoacetate (8.5 mg, 5.3 μL, 1.1 Eq, 55 μmol) was added and the mixture was stirred for 1 h. Sat. NH₄Cl and DCM were added and the layers were separated. The organic layer was washed with brine, dried (MgSO₄) and concentrated in vacuo to afford crude methyl 2-(3-((6-((4-methoxyphenyl)sulfonyl)-1-oxophthalazin-2(1H)-yl)methyl)-1H-pyrazol-1-yl)acetate. The product was used without further purification and characterization in the next step.

### Step 2

LiBH₄ (2M in THF) (1.7 mg, 38 μL, 2 molar, 1.5 Eq, 77 μmol) was added to a solution of methyl 2-(3-((6-((4-methoxyphenyl)sulfonyl)-1-oxophthalazin-2(1H)-yl)methyl)-1H-pyrazol-1-yl)acetate (24 mg, 1 Eq, 51 μmol) in THF (0.25 mL) under N₂ and the mixture was stirred for 2 h at RT. Further LiBH₄ (2M in THF) (38 μL, 2 molar, 1.5 Eq, 77 μmol) was added and the mixture stirred for 30 minutes. Sat. NH₄Cl and DCM were added and the layers were separated. The organic layer was washed with brine, then absorbed on silica. The crude product was purified by chromatography on silica gel to afford 2-((1-(2-hydroxyethyl)-1H-pyrazol-3-yl)methyl)-6-((4-methoxyphenyl)sulfonyl)phthalazin-1(2H)-one (8 mg). The product was dissolved in DMSO (1 mL), filtered and purified by reversed phase preparative HPLC (Basic method (B)). The relevant fractions were evaporated in a Genevac to afford 2-((1-(2-hydroxyethyl)-1H-pyrazol-3-yl)methyl)-6-((4-methoxyphenyl)sulfonyl)phthalazin-1(2H)-one (1.5 mg) as a white solid. ¹H NMR (CDCl3) δ: 8.52 (d, J=8.4 Hz, 1H), 8.29 (d, J=1.8 Hz, 1H), 8.23 (s, 1H), 8.15 (dd, J=8.4, 1.7 Hz, 1H), 7.95-7.84 (m, 2H), 7.34 (d, J=2.3 Hz, 1H), 7.04-6.96 (m, 2H), 6.28 (d, J=2.3 Hz, 1H), 5.42 (s, 2H), 4.24-4.14 (m, 2H), 4.01-3.93 (m, 2H), 3.85 (s, 3H) (exchangeable OH not observed). MS (ES⁺): 441 (M+H)⁺.

### Example 25—2-((5-cyclopropyl-1H-pyrazol-3-yl)methyl)-6-(phenylsulfonyl)phthalazin-1(2H)-one

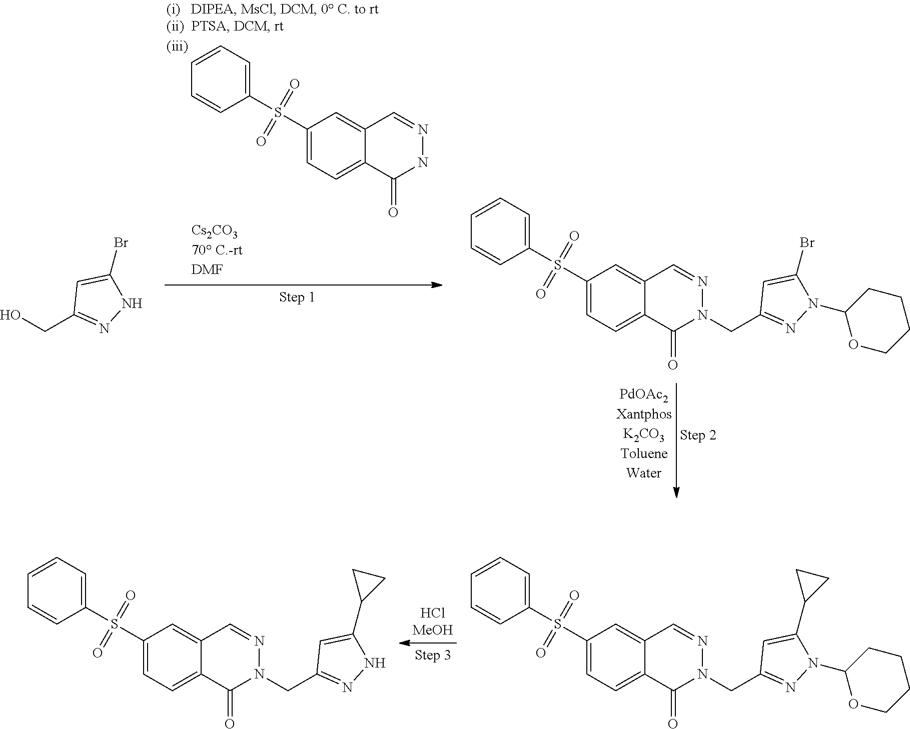

### Step 1

(i) Ms-Cl (295 mg, 201 μL, 1.1 Eq, 2.58 mmol) was added dropwise to a solution of (5-bromo-1H-pyrazol-3-yl)methanol hydrochloride (500 mg, 1 Eq, 2.34 mmol) and DIPEA (666 mg, 898 μL, 2.2 Eq, 5.15 mmol) in DCM (10 mL) under nitrogen at 0° C. and the mixture was stirred overnight. 1N HCl and DCM were added and the layers separated. The organic extract was washed with brine, dried (MgSO₄) and concentrated in vacuo to afford crude 5-bromo-3-(chloromethyl)-1H-pyrazole (338 mg, 1.73 mmol, 73.8%) as an oil. The product was used without further characterization and purification in the next step.

(ii) A mixture of 5-bromo-3-(chloromethyl)-1H-pyrazole (270 mg, 1 Eq, 1.38 mmol), pTSA·H₂O (263 mg, 1 Eq, 1.38 mmol) and 3,4-dihydro-2H-pyran (116 mg, 126 μL, 1 Eq, 1.38 mmol) in DCM (5 mL) was stirred overnight. Sat. NaHCO₃ and DCM were added and the layers separated. The organic extract was washed with brine, dried (MgSO₄) and concentrated in vacuo to afford the title compound (386 mg, 1.38 mmol, 99.9%) as a brown oil. The product was used without further purification and characterization in the next step. Quantitative yield assumed.

(iii) A mixture of 6-(phenylsulfonyl)phthalazin-1(2H)-one (Intermediate 2, 395 mg, 1 Eq, 1.38 mmol) and cesium carbonate (1.35 g, 3 Eq, 4.14 mmol) in DMF (7 mL) was stirred at 70° C. for 45 minutes. 5-bromo-3-(chloromethyl)-1-(tetrahydro-2H-pyran-2-yl)-1H-pyrazole (386 mg, 1 Eq, 1.38 mmol) was added and the mixture was allowed to cool to RT and stirred overnight. Water and DCM were added and the layers separated. The organic layer was washed with brine, then absorbed on silica. The crude product was purified by chromatography on silica gel. The product was dissolved in DCM and the resulting solution was washed with water, then brine, dried (MgSO₄) and concentrated in vacuo to afford 2-((5-bromo-1-(tetrahydro-2H-pyran-2-yl)-1H-pyrazol-3-yl)methyl)-6-(phenylsulfonyl)phthalazin-1(2H)-one (71 mg) as a white solid. MS (ES⁺): 551/553 (M+Na)⁺.

### Step 2

N₂ was bubbled through a mixture of 2-((5-bromo-1-(tetrahydro-2H-pyran-2-yl)-1H-pyrazol-3-yl)methyl)-6-(phenylsulfonyl)phthalazin-1(2H)-one (71 mg, 88% Wt, 1 Eq, 0.12 mmol), Potassium cyclopropyl trifluoroborate (31 mg, 1.75 Eq, 0.21 mmol) and potassium carbonate (57 mg, 3.5 Eq, 0.41 mmol) in toluene (0.75 mL):water (0.25 mL) for 5 minutes. Palladium (II) acetate (3.3 mg, 0.125 Eq, 15 μmol) and Xantphos (16 mg, 0.24 Eq, 28 μmol) were added and the mixture was stirred at 100° C. under nitrogen for 1 h, then allowed to cool to RT. Water and DCM were added and the layers separated. The organic extract was washed with brine, then absorbed on silica. The crude product was purified by chromatography on silica gel to afford 2-((5-cyclopropyl-1-(tetrahydro-2H-pyran-2-yl)-1H-pyrazol-3-yl)methyl)-6-(phenylsulfonyl)phthalazin-1(2H)-one (29 mg) as a pale yellow solid. MS (ES⁺): 537 (M+H)⁺.

### Step 3

Hydrogen chloride (4N in dioxane) (22 mg, 0.15 mL, 4 molar, 11.5 Eq, 0.60 mmol) was added to a solution of 2-((5-cyclopropyl-1-(tetrahydro-2H-pyran-2-yl)-1H-pyrazol-3-yl)methyl)-6-(phenylsulfonyl)phthalazin-1(2H)-one (29 mg, 88% Wt, 1 Eq, 52 μmol) in MeOH (0.5 mL) and the mixture was stirred for 1.45 h, then concentrated in vacuo. Sat. NaHCO₃ and DCM were added and the layers separated. The organic layer was washed with brine, then absorbed on silica. The crude product was purified by chromatography on silica gel to afford the title compound (14.7 mg) as a white solid. ¹H NMR (DMSO-d6) δ: 12.35 (s, 1H), 8.69 (d, J=1.9 Hz, 1H), 8.60 (s, 1H), 8.43 (d, J=8.4 Hz, 1H), 8.31 (dd, J=8.4, 1.5 Hz, 1H), 8.08-8.00 (m, 2H), 7.78-7.72 (m, 1H), 7.72-7.60 (m, 2H), 5.76 (s, 1H), 5.19 (s, 2H), 1.86-1.74 (m, 1H), 0.88-0.81 (m, 2H), 0.63-0.51 (m, 2H). MS (ES⁺): 407 (M+H)⁺.

### Example 26—2-((4-(hydroxymethyl)-1H-pyrazol-3-yl)methyl)-6-(phenylsulfonyl)phthalazin-1(2H)-one

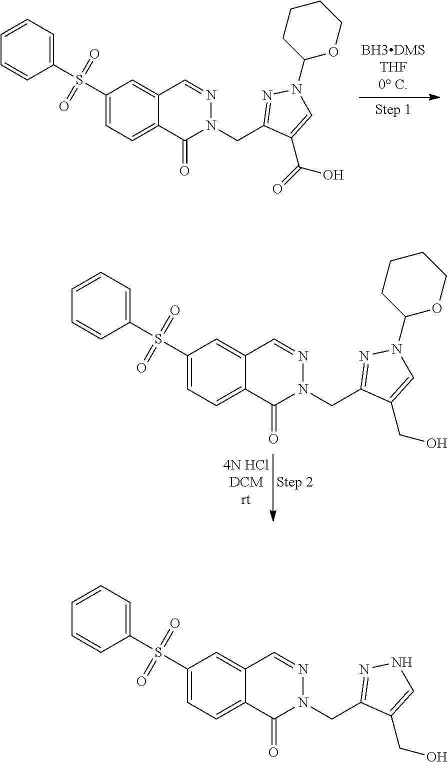 [SMILES: O=C(O)c1cn(C2CCCCO2)nc1Cn1ncc2cc(S(=O)(=O)c3ccccc3)ccc2c1=O ; O=c1c2ccc(S(=O)(=O)c3ccccc3)cc2cnn1Cc1nn(C2CCCCO2)cc1CO ; O=c1c2ccc(S(=O)(=O)c3ccccc3)cc2cnn1Cc1n[nH]cc1CO]

### Step 1

Borane-methyl sulfide complex (34.6 mg, 228 μL, 2 molar, 2 Eq, 456 μmol) was added to a cooled solution (0° C.) of 3-((1-oxo-6-(phenylsulfonyl)phthalazin-2(1H)-yl)methyl)-1-(tetrahydro-2H-pyran-2-yl)-1H-pyrazole-4-carboxylic acid (Example 15 Intermediate, 179 mg, 63% Wt, 1 Eq, 228 μmol) in dry THF (3.0 mL). The reaction mixture was stirred for 2 h at 0° C., then allowed to warm to RT and stirred for further 2 h. The reaction mixture was diluted with water (25 mL) and transferred into a separating funnel. The crude product was extracted with EtOAc (2×25 mL and the combined organic layers were dried (MgSO₄), filtered and concentrated in vacuo. The crude product was purified by chromatography on silica gel to afford 2-((4-(hydroxymethyl)-1-(tetrahydro-2H-pyran-2-yl)-1H-pyrazol-3-yl)methyl)-6-(phenylsulfonyl)phthalazin-1(2H)-one (44 mg) as a clear white solid. MS (ES⁺): 463 (M+H)⁺.

### Step 2

2-((4-(hydroxymethyl)-1-(tetrahydro-2H-pyran-2-yl)-1H-pyrazol-3-yl)methyl)-6-(phenylsulfonyl)phthalazin-1(2H)-one (30.0 mg, 1 Eq, 62.4 μmol) was dissolved in DCM (3 mL) and treated with HCl (4N in dioxane) (6.83 mg, 46.8 μL, 4 molar, 3 Eq, 187 μmol). The reaction was stirred for 20 h at RT, then diluted with DCM (5 mL) and washed with sat. aq. NaHCO₃ (5 mL). The organic layer was collected and the aqueous was extracted with DCM (5 mL). The combined organic extracts were dried and concentrated in vacuo to afford the crude product. The crude product was dissolved in 2.5 mL DMSO/MeOH, filtered and purified by reversed phase preparative HPLC (Basic method). The relevant fractions were combined and evaporated in a Genevac, yielding the title compound (3.0 mg) as a clear white solid. ¹H NMR (400 MHz, DMSO-d6) δ 12.44 (s, 1H), 8.67 (d, J=1.8 Hz, 1H), 8.58 (s, 1H), 8.43 (d, J=8.4 Hz, 1H), 8.33-8.26 (m, 1H), 8.06-7.99 (m, 2H), 7.79-7.70 (m, 1H), 7.70-7.63 (m, 2H), 7.53 (s, 1H), 5.32 (s, 2H), 4.60 (t, J=5.3 Hz, 1H), 4.36 (d, J=5.3 Hz, 2H). MS (ES⁺): 397 (M+H)⁺.

### Example 27—2-((2,3-dihydropyrazolo[5,1-b]oxazol-6-yl)methyl)-6-(phenylsulfonyl)phthalazin-1(2H)-one

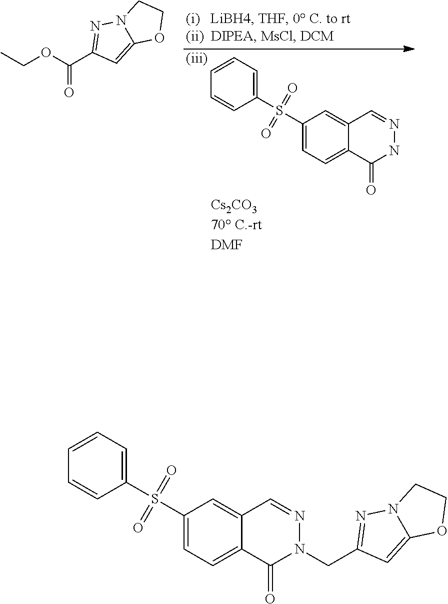

### Step 1

Lithium borohydride (2M in THF) (14.3 mg, 329 μL, 2 molar, 1.2 Eq, 659 μmol) was added to a solution of ethyl 2,3-dihydropyrazolo[5,1-b]oxazole-6-carboxylate (100 mg, 1 Eq, 549 μmol) in THF (2.5 mL) at 0° C. under N₂ and the mixture was stirred for 3 days. 1 N HCl and DCM were added and the layers separated. The organic extract was washed with brine, dried (MgSO₄) and concentrated in vacuo to afford crude (2,3-dihydropyrazolo[5,1-b]oxazol-6-yl)methanol (22 mg, 0.16 mmol) as a colourless oil. The product was used without further purification in the next step.

### Step 2

Ms-Cl (20 mg, 13 μL, 1.1 Eq, 0.17 mmol) was added to a solution of (2,3-dihydropyrazolo[5,1-b]oxazol-6-yl)methanol (22 mg, 1 Eq, 0.16 mmol) and DIPEA (22 mg, 30 μL, 1.1 Eq, 0.17 mmol) in DCM (1 mL) and the mixture was stirred overnight. 1 N HCl and DCM were added and the layers separated. The organic extract was washed with brine, dried (MgSO₄) and concentrated in vacuo to afford crude 6-(chloromethyl)-2,3-dihydropyrazolo[5,1-b]oxazole (25 mg, 0.16 mmol, 100%) as an oil. The product was used without further purification and characterization in the next step.

### Step 3

A mixture of 6-(phenylsulfonyl)phthalazin-1(2H)-one (22 mg, 1 Eq, 76 μmol) and cesium carbonate (49 mg, 2 Eq, 0.15 mmol) in DMF (1 mL) was stirred at 70° C. for 45 minutes. 6-(chloromethyl)-2,3-dihydropyrazolo[5,1-b]oxazole (Intermediate 2, 12 mg, 1 Eq, 76 μmol) was added and the mixture was allowed to cool to RT and stirred overnight. Water and DCM were added and the layers separated. The organic layer was washed with brine, then absorbed on silica. The crude product was purified by chromatography on silica gel to afford the title compound (2.8 mg) as a white solid. ¹H NMR (DMSO-d6) δ: 8.68 (d, J=1.9 Hz, 1H), 8.59 (d, J=0.7 Hz, 1H), 8.46-8.39 (m, 1H), 8.30 (dd, J=8.5, 1.9 Hz, 1H), 8.07-7.99 (m, 2H), 7.79-7.70 (m, 1H), 7.70-7.62 (m, 2H), 5.30 (s, 1H), 5.15 (s, 2H), 5.02-4.94 (m, 2H), 4.23-4.12 (m, 2H). MS (ES⁺): 409 (M+H)⁺.

### Example 28—2-((1-(difluoromethyl)-1H-pyrazol-3-yl)methyl)-6-(phenylsulfonyl)phthalazin-1(2H)-one

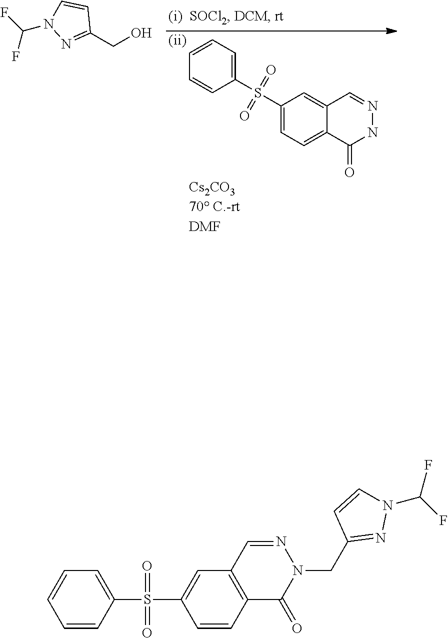

### Step 1

A stirred solution of (1-(difluoromethyl)-1H-pyrazol-3-yl)methanol (100 mg, 69.9 μL, 95% Wt, 1 Eq, 641 μmol) in DCM (3 mL) was treated with thionyl chloride (88.0 mg, 54.0 μL, 1.15 Eq, 740 μmol) dropwise. The resultant suspension was stirred at RT for 24 h. The reaction mixture was concentrated in vacuo and the residue was azeotroped with toluene (2 times) to afford the crude product 3-(chloromethyl)-1-(difluoromethyl)-1H-pyrazole, HCl as a colourless oil. The crude was used without purification or analysis. Quantitative yield assumed.

### Step 2

A stirred suspension of 6-(phenylsulfonyl)phthalazin-1(2H)-one (Intermediate 2, 185 mg, 1.01 Eq, 646 μmol) and cesium carbonate (627 mg, 3.0 Eq, 1.92 mmol) in DMF (3 mL) was heated to 80° C. for 1 h and then allowed to cool to RT. A solution of 3-(chloromethyl)-1-(difluoromethyl)-1H-pyrazole, HCl (130 mg, 1 Eq, 641 μmol) in DMF (1 mL) was added dropwise. The reaction mixture was stirred at RT for 18 h. The reaction mixture was concentrated in vacuo and azeotroped with toluene (2 times) to afford the crude product. The crude product was purified by chromatography on silica gel to afford impure product. The crude product was purified by chromatography on silica gel to afford the title compound (20.7 mg) as a white solid. ¹H NMR (400 MHz, DMSO-d6) δ 8.70 (d, J=1.8 Hz, 1H), 8.63 (s, 1H), 8.44 (d, J=8.4 Hz, 1H), 8.32 (dd, J=8.4, 1.9 Hz, 1H), 8.14 (d, J=2.7 Hz, 1H), 8.06-8.00 (m, 2H), 7.77-7.73 (m, 1H), 7.72 (t, J=59.0 Hz, 1H), 7.69-7.64 (m, 2H), 6.41 (d, J=2.6 Hz, 1H), 5.34 (s, 2H). MS (ES⁺): 417 (M+H)⁺.

The following compounds were made using similar procedures described above:

| Example | | |
|---|---|---|
| No. | Example Structure/Name | LCMS/¹H NMR data |
| 29 | 2-((2,3-dihydrobenzo[b][1,4]dioxin-6- | MS (ES⁺): 435 (M + H)⁺ |
| | yl)methyl)-6- | ¹H NMR (DMSO-d6) δ: 8.68 (d, J = 1.8 |
| | (phenylsulfonyl)phthalazin-1(2H)-one | Hz, 1H), 8.61 (s, 1H), 8.42 (d, J = 8.4 Hz, |
| | | 1H), 8.30 (dd, J = 8.4, 1.8 Hz, 1H), 8.08 − |
| | 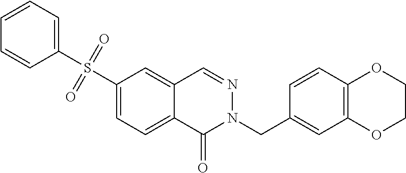 [SMILES: O=c1c2ccc(S(=O)(=O)c3ccccc3)cc2cnn1Cc1ccc2c(c1)OCCO2] | 7.99 (m, 2H), 7.78 − 7.70 (m, 1H), 7.70 − 7.61 (m, 2H), 6.84 − 6.79 (m, 1H), 6.82 − 6.73 (m, 2H), 5.19 (s, 2H), 4.18 (s, 4H). |
| 30 | 2-((2-methylthiazol-5-yl)methyl)-6- | MS (ES⁺): 398 (M + H)⁺ |
| | (phenylsulfonyl)phthalazin-1(2H)-one | ¹H NMR (DMSO-d6) δ: 8.69 (d, J = 1.8 |
| | | Hz, 1H), 8.64 (s, 1H), 8.43 (d, J = 8.4 Hz, |
| | 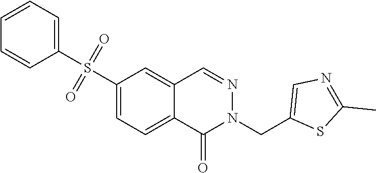 [SMILES: Cc1ncc(Cn2ncc3cc(S(=O)(=O)c4ccccc4)ccc3c2=O)s1] | 1H), 8.31 (dd, J = 8.5, 1.9 Hz, 1H), 8.05 − 7.99 (m, 2H), 7.77 − 7.71 (m, 1H), 7.69 − 7.62 (m, 3H), 5.45 (s, 2H), 2.56 (s, 3H). |
| 31 | 2-((5-methoxypyridin-3-yl)methyl)-6- | MS (ES⁺): 408 (M + H)⁺ |
| | (phenylsulfonyl)phthalazin-1(2H)-one | ¹H NMR (400 MHz, DMSO-d6) δ 8.69 (d, |
| | | J = 1.8 Hz, 1H), 8.64 (s, 1H), 8.44 (d, J = |
| | 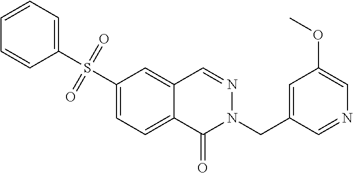 [SMILES: COc1cncc(Cn2ncc3cc(S(=O)(=O)c4ccccc4)ccc3c2=O)c1] | 8.4 Hz, 1H), 8.32 (dd, J = 8.4, 1.9 Hz, 1H), 8.20 (d, J = 2.8 Hz, 1H), 8.15 (d, J = 1.7 Hz, 1H), 8.05 − 8.01 (m, 2H), 7.78 − 7.71 (m, 1H), 7.66 (dd, J = 8.3, 6.7 Hz, 2H), 7.30 (dd, J = 2.8, 1.8 Hz, 1H), 5.34 (s, 2H), 3.78 (s, 3H). MS (ES⁺): 408 (M + H)⁺ |
| 32 | 2-(2-fluorobenzyl)-6- | MS (ES⁺): 395 (M + H)⁺ |
| | (phenylsulfonyl)phthalazin-1(2H)-one | ¹H NMR (400 MHz, DMSO-d6) δ 8.70 (d, |
| | | J = 1.8 Hz, 1H), 8.63 (d, J = 0.6 Hz, 1H), |
| | 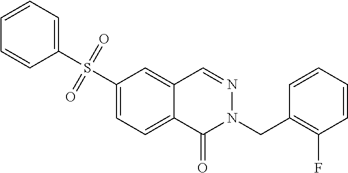 [SMILES: O=c1c2ccc(S(=O)(=O)c3ccccc3)cc2cnn1Cc1ccccc1F] | 8.44 (d, J = 8.4 Hz, 1H), 8.32 (dd, J = 8.4, 1.8 Hz, 1H), 8.06 − 8.01 (m, 2H), 7.78 − 7.70 (m, 1H), 7.70 − 7.63 (m, 2H), 7.33 (tdd, J = 7.3, 5.3, 1.8 Hz, 1H), 7.25 (td, J = 7.7, 1.8 Hz, 1H), 7.20 (ddd, J = 9.7, 8.3, 1.2 Hz, 1H), 7.11 (td, J = 7.4, 1.2 Hz, 1H), 5.38 (s, 2H). |
| 33 | 2-(2-fluoro-5-methoxybenzyl)-6- | MS (ES⁺): 425 (M + H)⁺ |
| | (phenylsulfonyl)phthalazin-1(2H)-one | ¹H NMR (DMSO-d6) δ: 8.69 (d, J = 1.8 |
| | | Hz, 1H), 8.62 (s, 1H), 8.44 (d, J = 8.4 Hz, |
| | 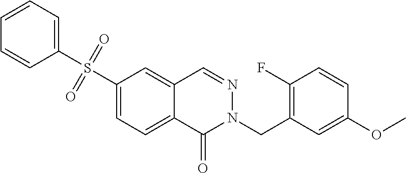 [SMILES: COc1ccc(F)c(Cn2ncc3cc(S(=O)(=O)c4ccccc4)ccc3c2=O)c1] | 1H), 8.32 (dd, J = 8.4, 1.8 Hz, 1H), 8.03 (d, J = 7.7, 1.7 Hz, 2H), 7.79 − 7.70 (m, 1H), 7.70 − 7.62 (m, 2H), 7.12 (dd, J = 9.3, 9.3 Hz, 1H), 6.87 (ddd, J = 9.0, 3.6, 3.6 Hz, 1H), 6.79 (dd, J = 6.1, 3.2 Hz, 1H), 5.33 (s, 2H). 3.65 (s, 3H). |
| 34 | 2-(imidazo[1,2-a]pyridin-5-ylmethyl)- | MS (ES⁺): 417 (M + H)⁺ |
| | 6-(phenylsulfonyl)phthalazin-1(2H)-one | ¹H NMR (DMSO-d6) δ: 8.71 (d, J = 1.8 |
| | | Hz, 1H), 8.66 (s, 1H), 8.48 (d, J = 8.4 Hz, |
| | 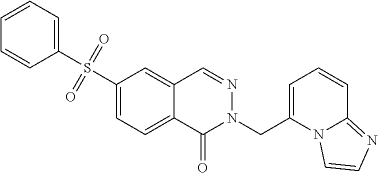 [SMILES: O=c1c2ccc(S(=O)(=O)c3ccccc3)cc2cnn1Cc1cccc2nccn12] | 1H), 8.35 (dd, J = 8.4, 1.9 Hz, 1H), 8.03 (dd, J = 7.2, 1.8 Hz, 2H), 7.99 (s, 1H), 7.79 − 7.71 (m, 1H), 7.71 − 7.61 (m, 3H), 7.57 (d, J = 9.1 Hz, 1H), 7.21 (dd, J = 9.1, 6.9 Hz, 1H), 6.82 (d, J = 6.9 Hz, 1H), 5.65 (s, 2H). |
| 35 | 2-((2,2-dimethyl-2,3- | MS (ES⁺): 447 (M + H)⁺ |
| | dihydrobenzofuran-5-yl)methyl)-6- | ¹H NMR (DMSO-d6) δ 8.68 (d, J = 1.9 |
| | (phenylsulfonyl)phthalazin-1(2H)-one | Hz, 1H), 8.61 (s, 1H), 8.43 (d, J = 8.5 Hz, |
| | | 1H), 8.30 (dd, J = 8.5, 1.9 Hz, 1H), 8.02 |
| | 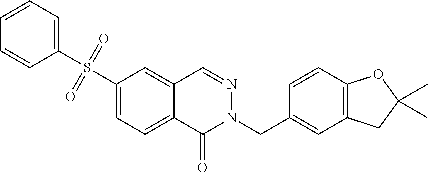 [SMILES: CC1(C)Cc2cc(Cn3ncc4cc(S(=O)(=O)c5ccccc5)ccc4c3=O)ccc2O1] | (d, J = 7.6 Hz, 2H), 7.78 − 7.70 (m, 1H), 7.70 − 7.62 (m, 2H), 7.15 (s, 1H), 7.07 (d, J = 8.1 Hz, 1H), 6.61 (d, J = 8.1 Hz, 1H), 5.21 (s, 2H), 2.93 (s, 2H), 1.35 (s, 6H). |
| 36 | 2-((1-methyl-1H-indazol-5-yl)methyl)- | MS (ES⁺): 431 (M + H)⁺ |
| | 6-(phenylsulfonyl)phthalazin-1(2H)- | ¹H NMR (400 MHz, DMSO-d6) δ 8.69 (d, |
| | | J = 1.8 Hz, 1H), 8.63 (s, 1H), 8.44 (d, J = |
| | 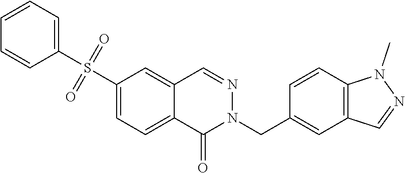 [SMILES: Cn1ncc2cc(Cn3ncc4cc(S(=O)(=O)c5ccccc5)ccc4c3=O)ccc21] | 8.5 Hz, 1H), 8.31 (dd, J = 8.4, 1.9 Hz, 1H), 8.02 (dd, J = 7.2, 1.7 Hz, 2H), 7.98 (d, J = 1.0 Hz, 1H), 7.77 − 7.70 (m, 1H), 7.70 − 7.63 (m, 3H), 7.57 (d, J = 8.7 Hz, 1H), 7.40 (dd, J = 8.7, 1.6 Hz, 1H), 5.42 (s, 2H), 3.99 (s, 3H). |
| 37 | 2-((6-methoxypyridin-2-yl)methyl)-6- | MS (ES⁺): 431 (M + H)⁺ |
| | (phenylsulfonyl)phthalazin-1(2H)-one | ¹H NMR (DMSO-d6) δ: 8.72 (d, J = 1.9 |
| | | Hz, 1H), 8.65 (s, 1H), 8.44 (d, J = 8.5 Hz, |
| | 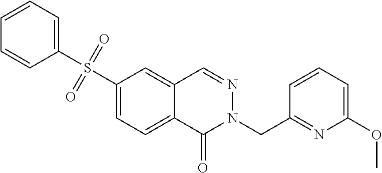 [SMILES: COc1cccc(Cn2ncc3cc(S(=O)(=O)c4ccccc4)ccc3c2=O)n1] | 1H), 8.33 (dd, J = 8.4, 1.9 Hz, 1H), 8.09 − 8.01 (m, 2H), 7.82 − 7.72 (m, 1H), 7.73 − 7.65 (m, 2H), 7.60 (dd, J = 8.3, 7.4 Hz, 1H), 6.74 (d, J = 7.3 Hz, 1H), 6.68 (d, J = 8.2 Hz, 1H), 5.35 (s, 2H), 3.68 (s, 3H). |
| 38 | 2-((6-aminopyridin-2-yl)methyl)-6- | MS (ES⁺): 393 (M + H)⁺ |
| | (phenylsulfonyl)phthalazin-1(2H)-one | ¹H NMR (DMSO-d6) δ: 8.71 (d, J = 1.8 |
| | | Hz, 1H), 8.63 (s, 1H), 8.44 (d, J = 8.4 Hz, |
| | 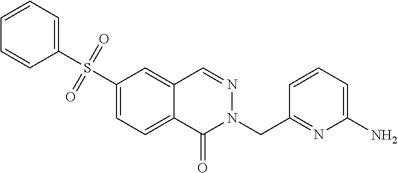 [SMILES: Nc1cccc(Cn2ncc3cc(S(=O)(=O)c4ccccc4)ccc3c2=O)n1] | 1H), 8.32 (dd, J = 8.4, 1.9 Hz, 1H), 8.12 − 8.00 (m, 2H), 7.79 − 7.71 (m, 1H), 7.71 − 7.63 (m, 3H), 7.31 − 7.26 (m, 1H), 6.36 − 6.29 (m, 1H), 6.00 (s (br), 2H), 5.19 (s, 2H). |
| 39 | 2-((1-methyl-1H-indazol-6-yl)methyl)- | MS (ES⁺): 431 (M + H)⁺ |
| | 6-(phenylsulfonyl)phthalazin-1(2H)- | ¹H NMR (400 MHz, DMSO-d6) δ 8.69 (d, |
| | one | J = 1.8 Hz, 1H), 8.63 (s, 1H), 8.44 (d, J = |
| | | 8.5 Hz, 1H), 8.31 (dd, J = 8.5, 1.8 Hz, |
| | 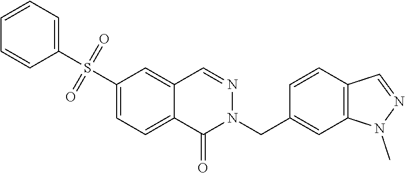 [SMILES: Cn1ncc2ccc(Cn3ncc4cc(S(=O)(=O)c5ccccc5)ccc4c3=O)cc21] | 1H), 8.06 − 7.99 (m, 2H), 7.97 (s, 1H), 7.78 − 7.70 (m, 1H), 7.70 − 7.62 (m, 3H), 7.57 (d, J = 8.7 Hz, 1H), 7.40 (dd, J = 8.7, 1.6 Hz, 1H), 5.42 (s, 2H), 3.99 (s, 3H). |
| 40 | 2-(3-(difluoromethoxy)benzyl)-6-((4- | MS (ES⁺): 473 (M + H)⁺ |
| | methoxyphenyl)sulfonyl)phthalazin- | ¹H NMR (400 MHz, DMSO-d6) δ 8.65 (d, |
| | 1(2H)-one | J = 1.8 Hz, 1H), 8.63 (d, J = 0.7 Hz, 1H), |
| | | 8.42 (d, J = 8.4 Hz, 1H), 8.28 (dd, J = |
| | 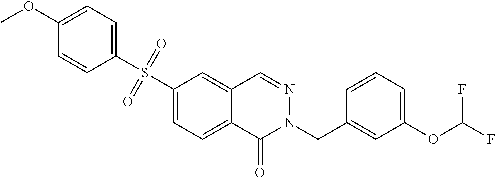 [SMILES: COc1ccc(S(=O)(=O)c2ccc3c(=O)n(Cc4cccc(OC(F)F)c4)ncc3c2)cc1] | 8.4, 1.9 Hz, 1H), 7.98 − 7.93 (m, 2H), 7.39 − 7.33 (m, 1H), 7.20 − 7.14 (m, 4H), 7.13 − 7.05 (m, 2H), 5.34 (s, 2H), 3.83 (s, 3H). |
| 41 | 2-((6-methoxypyridin-3-yl)methyl)-6- | MS (ES⁺): 408 (M + H)⁺ |
| | (phenylsulfonyl)phthalazin-1(2H)-one | ¹H NMR (400 MHz, DMSO-d6) δ 8.68 (d, |
| | | J = 1.8 Hz, 1H), 8.62 (s, 1H), 8.43 (d, J = |
| | 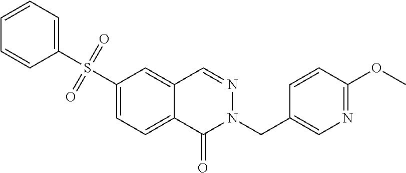 [SMILES: COc1ccc(Cn2ncc3cc(S(=O)(=O)c4ccccc4)ccc3c2=O)cn1] | 8.4 Hz, 1H), 8.30 (dd, J = 8.5, 1.9 Hz, 1H), 8.19 (d, J = 2.4 Hz, 1H), 8.05 − 7.99 (m, 2H), 7.77 − 7.70 (m, 1H), 7.70 − 7.63 (m, 3H), 6.75 (d, J = 8.5 Hz, 1H), 5.27 (s, 2H), 3.80 (s, 3H). |
| 42 | 6-((4-methoxyphenyl)sulfonyl)-2- | MS (ES⁺): 408 (M + H)⁺ |
| | (pyridin-3-ylmethyl)phthalazin-1(2H)- | ¹H NMR (400 MHz, DMSO-d6) δ 8.64 (d, |
| | one | J = 1.9 Hz, 1H), 8.63 (s, 1H), 8.57 (d, J = |
| | | 2.2 Hz, 1H), 8.48 (dd, J = 4.8, 1.6 Hz, |
| | 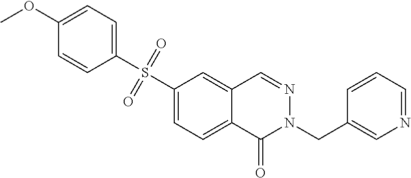 [SMILES: COc1ccc(S(=O)(=O)c2ccc3c(=O)n(Cc4cccnc4)ncc3c2)cc1] | 1H), 8.42 (d, J = 8.4 Hz, 1H), 8.28 (dd, J = 8.4, 1.8 Hz, 1H), 7.97 − 7.92 (m, 2H), 7.71 (dt, J = 8.0, 2.0 Hz, 1H), 7.33 (ddd, J = 7.9, 4.8, 0.9 Hz, 1H), 7.19 − 7.13 (m, 2H), 5.36 (s, 2H), 3.83 (s, 3H). |
| 43 | 2-(2-aminobenzyl)-6-((4- | MS (ES⁺): 422 (M + H)⁺ |
| | methoxyphenyl)sulfonyl)phthalazin- | ¹H NMR (400 MHz, DMSO-d6) δ 8.67 − |
| | 1(2H)-one | 8.62 (m, 2H), 8.44 (d, J = 8.5 Hz, 1H), |
| | | 8.28 (dd, J = 8.4, 1.8 Hz, 1H), 7.99 − |
| | 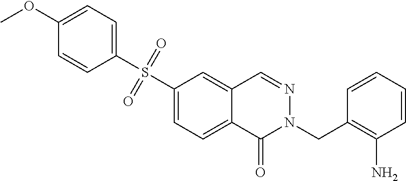 [SMILES: COc1ccc(S(=O)(=O)c2ccc3c(=O)n(Cc4ccccc4N)ncc3c2)cc1] | 7.91 (m, 2H), 7.19 − 7.14 (m, 2H), 7.01 − 6.91 (m, 2H), 6.64 (dd, J = 7.9, 1.2 Hz, 1H), 6.48 (td, J = 7.4, 1.2 Hz, 1H), 5.20 (s, 2H), 5.17 (s, 2H), 3.83 (s, 3H). |
| 44 | 6-((4-methoxyphenyl)sulfonyl)-2- | MS (ES⁺): 421 (M + H)⁺ |
| | phenethylphthalazin-1(2H)-one | ¹H NMR (400 MHz, DMSO-d6) δ 8.62 (d, |
| | | J = 1.8 Hz, 1H), 8.58 (s, 1H), 8.39 (d, J = |
| | 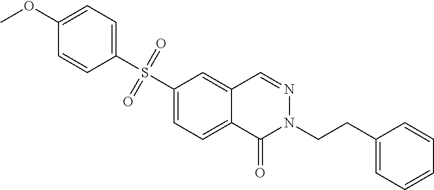 [SMILES: COc1ccc(S(=O)(=O)c2ccc3c(=O)n(CCc4ccccc4)ncc3c2)cc1] | 8.4 Hz, 1H), 8.26 (dd, J = 8.5, 1.8 Hz, 1H), 7.99 − 7.93 (m, 2H), 7.28 − 7.23 (m, 2H), 7.21 − 7.14 (m, 5H), 4.36 (t, J = 8.3, 6.7 Hz, 2H), 3.84 (s, 3H), 3.05 (t, J = 7.4, 7.4 Hz, 2H). |
| 45 | 2-(isoxazol-5-ylmethyl)-6-((4- | MS (ES⁺): 398 (M + H)⁺ |
| | methoxyphenyl)sulfonyl)phthalazin- | ¹H NMR (400 MHz, DMSO-d6) δ 8.66 (d, |
| | 1(2H)-one | J = 1.8 Hz, 1H), 8.64 (s, 1H), 8.51 (d, |
| | | J = 1.9 Hz, 1H), 8.42 (d, J = 8.4 Hz, 1H), |
| | 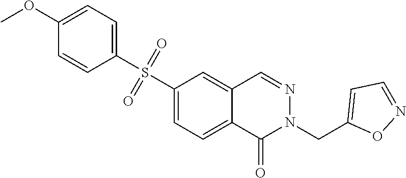 [SMILES: COc1ccc(S(=O)(=O)c2ccc3c(=O)n(Cc4ccno4)ncc3c2)cc1] | 8.29 (dd, J = 8.4, 1.8 Hz, 1H), 7.98 − 7.93 (m, 2H), 7.20 − 7.13 (m, 2H), 6.45 (d, J = 1.8 Hz, 1H), 5.51 (s, 2H), 3.83 (s, 3H). |
| 46 | 2-(4-chloro-2-fluorobenzyl)-6-((4- | MS (ES⁺): 459/461 (M + H)⁺ |
| | methoxyphenyl)sulfonyl)phthalazin- | ¹H NMR (DMSO-d6) δ: 8.64 (d, 1H), |
| | 1(2H)-one | 8.62 (s, 1H), 8.41 (d, 1H), 8.28 (dd, 1H), |
| | | 7.98 − 7.92 (m, 2H), 7.44 (dd, 1H), 7.31 |
| | 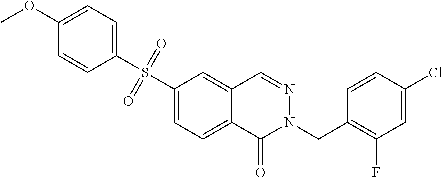 [SMILES: COc1ccc(S(=O)(=O)c2ccc3c(=O)n(Cc4ccc(Cl)cc4F)ncc3c2)cc1] | (dd, 1H), 7.21 (dd, 1H), 7.20 − 7.14 (m, 2H), 5.35 (s, 2H), 3.83 (s, 3H). |
| 47 | 2-((2,3-dihydrobenzofuran-5- | MS (ES⁺): 449 (M + H)⁺ |
| | yl)methyl)-6-((4- | ¹H NMR (400 MHz, DMSO-d6) δ 8.65 − |
| | methoxyphenyl)sulfonyl)phthalazin- | 8.57 (m, 2H), 8.41 (d, 1H), 8.26 (dd, |
| | 1(2H)-one | 1H), 7.99 − 7.90 (m, 2H), 7.23 − 7.13 |
| | | (m, 3H), 7.07 (d, 1H), 6.67 (d, 1H), 5.22 |
| | 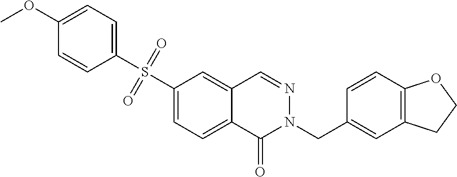 [SMILES: COc1ccc(S(=O)(=O)c2ccc3c(=O)n(Cc4ccc5c(c4)CCO5)ncc3c2)cc1] | (s, 2H), 4.46 (t, 2H), 3.83 (s, 3H), 3.10 (t, 2H). |
| 48 | 2-((1H-indazol-3-yl)methyl)-6-((4- | MS (ES⁺): 447 (M + H)⁺ |
| | methoxyphenyl)sulfonyl)phthalazin- | ¹H NMR (400 MHz, DMSO-d6) δ 12.94 |
| | 1(2H)-one | (s, 1H), 8.62 (d, J = 1.8 Hz, 1H), 8.60 (d, |
| | | J = 0.7 Hz, 1H), 8.45 (d, J = 8.4 Hz, 1H), |
| | 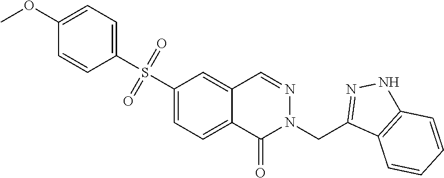 [SMILES: COc1ccc(S(=O)(=O)c2ccc3c(=O)n(Cc4n[nH]c5ccccc45)ncc3c2)cc1] | 8.27 (dd, J = 8.4, 1.9 Hz, 1H), 7.97 − 7.91 (m, 2H), 7.74 − 7.69 (m, 1H), 7.48 (dt, J = 8.4, 1.0 Hz, 1H), 7.30 (ddd, J = 8.3, 6.8, 1.1 Hz, 1H), 7.18 − 7.13 (m, 2H), 7.05 (ddd, J = 7.9, 6.8, 0.9 Hz, 1H), 5.66 (s, 2H), 3.82 (s, 3H). |
| 49 | 2-(4-methoxybenzyl)-6- | MS (ES⁺): 407 (M + H)⁺ |
| | (phenylsulfonyl)phthalazin-1(2H)-one | ¹H NMR (400 MHz, DMSO-d6) δ 8.64 (d, |
| | | J = 1.8 Hz, 1H), 8.63 (s, 1H), 8.42 (d, J = |
| | 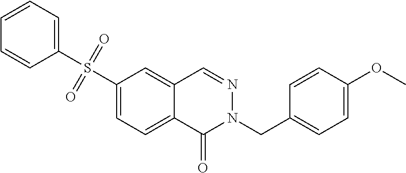 [SMILES: COc1ccc(Cn2ncc3cc(S(=O)(=O)c4ccccc4)ccc3c2=O)cc1] | 8.4 Hz, 1H), 8.27 (dd, J = 8.4, 1.9 Hz, 1H), 7.99 − 7.91 (m, 2H), 7.35 − 7.22 (m, 5H), 7.20 − 7.12 (m, 2H), 5.33 (s, 2H), 3.83 (s, 3H). |
| 50 | 2-benzyl-6-((4- | MS (ES⁺): 407 (M + H)⁺ |
| | methoxyphenyl)sulfonyl)phthalazin- | ¹H NMR (400 MHz, DMSO-d6) δ 8.68 (d, |
| | 1(2H)-one | J = 1.8 Hz, 1H), 8.62 (s, 1H), 8.43 (d, J = |
| | | 8.4 Hz, 1H), 8.30 (dd, J = 8.5, 1.9 Hz, |
| | 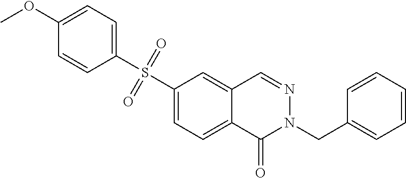 [SMILES: COc1ccc(S(=O)(=O)c2ccc3c(=O)n(Cc4ccccc4)ncc3c2)cc1] | 1H), 8.06 − 7.98 (m, 2H), 7.78 − 7.70 (m, 1H), 7.70 − 7.62 (m, 2H), 7.30 − 7.23 (m, 2H), 6.89 − 6.82 (m, 2H), 5.24 (s, 2H), 3.70 (s, 3H). |
| 51 | 6-((4-methoxyphenyl)sulfonyl)-2-((6- | MS (ES⁺): 438 (M + H)⁺ |
| | methoxypyridin-3- | ¹H NMR (400 MHz, DMSO-d6) δ 8.62 (d, |
| | yl)methyl)phthalazin-1(2H)-one | J = 1.9 Hz, 1H), 8.61 (d, J = 0.7 Hz, 1H), |
| | | 8.41 (d, J = 8.4 Hz, 1H), 8.27 (dd, J = |
| | 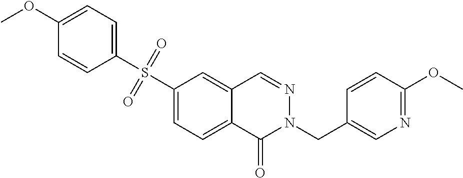 | 8.4, 1.9 Hz, 1H), 8.21 − 8.16 (m, 1H), 7.98 − 7.91 (m, 2H), 7.67 (dd, J = 8.6, 2.5 Hz, 1H), 7.20 − 7.13 (m, 2H), 6.76 (dd, J = 8.6, 0.7 Hz, 1H), 5.26 (s, 2H), 3.83 (s, 3H), 3.80 (s, 3H). |
| 52 | 2-(2-fluoro-3-methoxybenzyl)-6-((4- | MS (ES⁺): 455 (M + H)⁺ |
| | methoxyphenyl)sulfonyl)phthalazin- | ¹H NMR (400 MHz, DMSO-d6) δ 8.64 (d, |
| | 1(2H)-one | J = 1.8 Hz, 1H), 8.61 (s, 1H), 8.41 (d, J = |
| | | 8.4 Hz, 1H), 8.28 (dd, J = 8.5, 1.9 Hz, |
| | 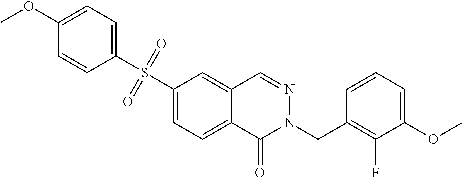 | 1H), 7.99 − 7.92 (m, 2H), 7.21 − 7.14 (m, 2H), 7.08 (td, J = 8.2, 1.8 Hz, 1H), 7.03 (td, J = 7.8, 1.1 Hz, 1H), 6.76 (td, J = 7.5, 6.9, 1.8 Hz, 1H), 5.36 (s, 2H), 3.83 (s, 3H), 3.81 (s, 3H). |
| 53 | 2-(2,6-difluoro-4-methoxybenzyl)-6- | MS (ES⁺): 473 (M + H)⁺ |
| | ((4- | ¹H NMR (DMSO-d6) δ: 8.59 (d, J = 1.8 |
| | methoxyphenyl)sulfonyl)phthalazin- | Hz, 1H), 8.54 (d, J = 0.7 Hz, 1H), 8.41 |
| | 1(2H)-one | (d, J = 8.4 Hz, 1H), 8.26 (dd, J = 8.4, |
| | | 1.9 Hz, 1H), 7.97 − 7.90 (m, 2H), 7.20 − |
| | 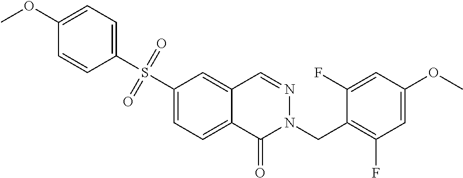 | 7.12 (m, 2H), 6.77 − 6.68 (m, 2H), 5.31 (s, 2H), 3.83 (s, 3H), 3.76 (s, 3H). |
| 54 | 6-((4-methoxyphenyl)sulfonyl)-2-((5- | MS (ES⁺): 411 (M + H)⁺ |
| | methyl-1H-pyrazol-3- | ¹H NMR (DMSO-d6) δ: 12.29 (s, 1H), |
| | yl)methyl)phthalazin-1(2H)-one | 8.63 (d, J = 1.8 Hz, 1H), 8.58 (s, 1H), |
| | | 8.41 (d, J = 8.4 Hz, 1H), 8.26 (dd, J = |
| | 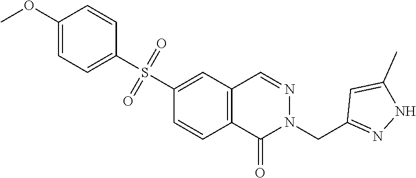 [SMILES: COc1ccc(S(=O)(=O)c2ccc3c(=O)n(Cc4cc(C)[nH]n4)ncc3c2)cc1] | 8.3, 1.3 Hz, 1H), 7.97 − 7.92 (m, 2H), 7.19 − 7.12 (m, 2H), 5.83 (s, 1H), 5.21 (s, 2H), 3.83 (s, 3H), 2.13 (s, 3H). |
| 55 | 2-((2-hydroxypyridin-4-yl)methyl)-6- | MS (ES⁺): 398 (M + H)⁺ |
| | ((4-methoxyphenyl) | ¹H NMR (DMSO-d6) δ: 11.48 (s, 1H), |
| | sulfonyl)phthalazin-1(2H)-one | 8.66 (d, J = 1.8 Hz, 1H), 8.65 (s, 1H), |
| | | 8.42 (d, J = 8.5 Hz, 1H), 8.29 (dd, J = |
| | 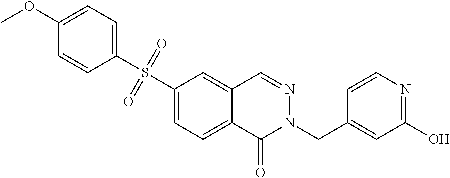 [SMILES: COc1ccc(S(=O)(=O)c2ccc3c(=O)n(Cc4ccnc(O)c4)ncc3c2)cc1] | 8.4, 1.8 Hz, 1H), 7.99 − 7.93 (m, 2H), 7.33 − 7.26 (m, 1H), 7.20 − 7.14 (m, 2H), 6.04 (d, J = 5.9 Hz, 2H), 5.14 (s, 2H), 3.84 (s, 3H). |
| 56 | 2-((2-hydroxypyridin-3-yl)methyl)-6- | MS (ES)⁺: 424 (M + H)⁺ |
| | ((4- | ¹H NMR (400 MHz, DMSO-d6) δ 11.70 |
| | methoxyphenyl)sulfonyl)phthalazin- | (s, 1H), 8.65 (d, J = 1.8 Hz, 1H), 8.62 (s, |
| | 1(2H)-one | 1H), 8.42 (d, J = 8.4 Hz, 1H), 8.28 (dd, |
| | | J = 8.5, 1.9 Hz, 1H), 7.99 − 7.93 (m, 2H), |
| | 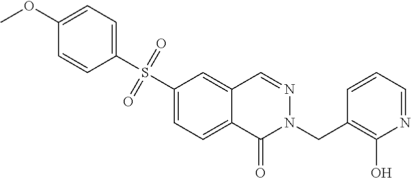 [SMILES: COc1ccc(S(=O)(=O)c2ccc3c(=O)n(Cc4cccnc4O)ncc3c2)cc1] | 7.30 (d, J = 6.5 Hz, 1H), 7.20 − 7.14 (m, 2H), 7.01 (dd, J = 6.8, 1.9 Hz, 1H), 6.06 (t, J = 6.7 Hz, 1H), 5.07 (s, 2H), 3.84 (s, 3H). |
| 57 | 2-(benzo[d][1,3]dioxol-5-ylmethyl)-6- | MS (ES)⁺: 421 (M + H)⁺ |
| | (phenylsulfonyl)phthalazin-1(2H)-one | ¹H NMR (DMSO-d6) δ: 8.68 (d, J = 1.8 |
| | | Hz, 1H), 8.62 (s, 1H), 8.43 (d, J = 8.5 Hz, |
| | 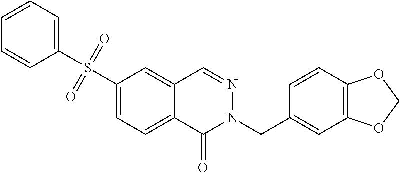 [SMILES: O=c1c2ccc(S(=O)(=O)c3ccccc3)cc2cnn1Cc1ccc2c(c1)OCO2] | 1H), 8.30 (dd, J = 8.4, 1.9 Hz, 1H), 8.07 − 7.99 (m, 2H), 7.78 − 7.70 (m, 1H), 7.70 − 7.62 (m, 2H), 6.89 (d, J = 1.5 Hz, 1H), 6.87 − 6.77 (m, 2H), 5.96 (s, 2H), 5.22 (s, 2H). |
| 58 | 2-((1H-indazol-6-yl)methyl)-6- | MS (ES)⁺: 417 (M + H)⁺ |
| | (phenylsulfonyl)phthalazin-1(2H)-one | ¹H NMR (400 MHz, DMSO-d6) δ 12.94 |
| | | (s, 1H), 8.70 (d, J = 1.8 Hz, 1H), 8.65 (s, |
| | 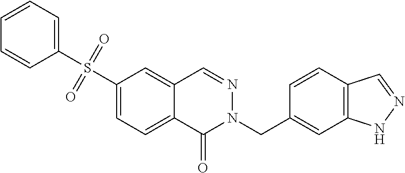 [SMILES: O=c1c2ccc(S(=O)(=O)c3ccccc3)cc2cnn1Cc1ccc2cn[nH]c2c1] | 1H), 8.45 (d, J = 8.4 Hz, 1H), 8.32 (dd, J = 8.4, 1.9 Hz, 1H), 8.06 − 7.99 (m, 3H), 7.77 − 7.71 (m, 1H), 7.67 (dt, J = 8.9, 6.3 Hz, 3H), 7.43 (s, 1H), 7.09 (dd, J = 8.3, 1.4 Hz, 1H), 5.45 (s, 2H). |
| 59 | 2-((5-methoxypyridin-2-yl)methyl)-6- | MS (ES)⁺: 408 (M + H)⁺ |
| | (phenylsulfonyl)phthalazin-1(2H)-one | ¹H NMR (DMSO) δ: 8.70 (d, J = 1.8 Hz, |
| | | 1H), 8.62 (s, 1H), 8.43 (d, J = 8.4 Hz, |
| | 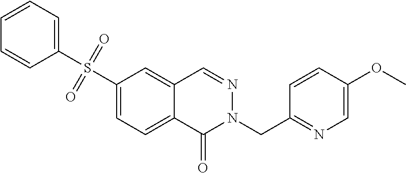 [SMILES: COc1ccc(Cn2ncc3cc(S(=O)(=O)c4ccccc4)ccc3c2=O)nc1] | 1H), 8.31 (dd, J = 8.4, 1.9 Hz, 1H), 8.15 (d, J = 2.9 Hz, 1H), 8.08 − 7.98 (m, 2H), 7.79 − 7.71 (m, 1H), 7.71 − 7.63 (m, 2H), 7.32 (dd, J = 8.6, 3.0 Hz, 1H), 7.23 (d, J = 8.7 Hz, 1H), 5.37 (s, 2H), 3.78 (s, 3H). |
| 60 | 2-((1-methyl-1H-pyrazol-3-yl)methyl)- | MS (ES⁺): 381 (M + H)⁺ |
| | 6-(phenylsulfonyl)phthalazin-1(2H)- | 1H NMR (400 MHz, DMSO-d6) δ 8.68 |
| | one | (d, J = 1.8 Hz, 1H), 8.59 (s, 1H), 8.43 (d, |
| | | J = 8.4 Hz, 1H), 8.30 (dd, J = 8.4, 1.9 Hz, |
| | 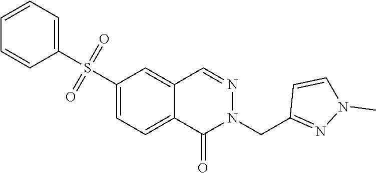 [SMILES: Cn1ccc(Cn2ncc3cc(S(=O)(=O)c4ccccc4)ccc3c2=O)n1] | 1H), 8.06 − 8.00 (m, 2H), 7.78 − 7.70 (m, 1H), 7.69 − 7.63 (m, 2H), 7.56 (d, J = 2.2 Hz, 1H), 6.09 (d, J = 2.2 Hz, 1H), 5.24 (s, 2H), 3.75 (s, 3H). |
| 61 | 2-(2-((1-oxo-6- | MS (ES⁺): 434 (M + H)⁺ |
| | (phenylsulfonyl)phthalazin-2(1H)- | ¹H NMR (DMSO-d6) δ: 8.70 (d, J = 1.8 |
| | yl)methyl)phenyl)acetamide | Hz, 1H), 8.64 (s, 1H), 8.43 (d, J = 8.4 Hz, |
| | | 1H), 8.32 (dd, J = 8.4, 1.8 Hz, 1H), 8.08 − |
| | 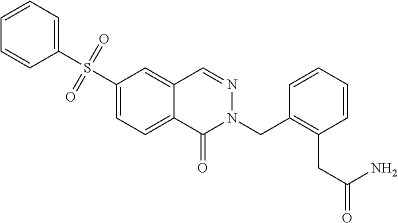 [SMILES: NC(=O)Cc1ccccc1Cn1ncc2cc(S(=O)(=O)c3ccccc3)ccc2c1=O] | 8.00 (m, 2H), 7.79 − 7.71 (m, 1H), 7.71 − 7.63 (m, 2H), 7.46 (s, 1H), 7.27 − 7.16 (m, 2H), 7.12 (dt, J = 7.4, 7.2, 1.6 Hz, 1H), 7.02 (d, J = 7.5 Hz, 1H), 6.92 (s, 1H), 5.40 (s, 2H), 3.62 (s, 2H). |
| 62 | 2-((6-(difluoromethoxy)pyridin-3- | MS (ES⁺): 444 (M + H)⁺ |
| | yl)methyl)-6- | ¹H NMR (DMSO-d6) δ: 8.69 (d, J = 1.8 |
| | (phenylsulfonyl)phthalazin-1(2H)-one | Hz, 1H), 8.63 (s, 1H), 8.43 (d, J = 8.5 Hz, |
| | | 1H), 8.31 (dd, J = 8.4, 1.9 Hz, 1H), 8.28 |
| | 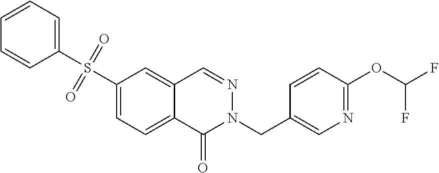 [SMILES: O=c1c2ccc(S(=O)(=O)c3ccccc3)cc2cnn1Cc1ccc(OC(F)F)nc1] | (d, J = 2.4 Hz, 1H), 8.06 − 7.99 (m, 2H), 7.87 (dd, J = 8.5, 2.5 Hz, 1H), 7.78 − 7.70 (m, 1H), 7.74 − 7.61 (m, 3H), 7.66 (t, J = 72.8 Hz, 1H), 7.03 (d, J = 8.4, 0.7 Hz, 1H), 5.34 (s, 2H). |
| 63 | 2-(2-(2-hydroxyethyl)benzyl)-6- | MS (ES⁺): 421 (M + H)⁺ |
| | (phenylsulfonyl)phthalazin-1(2H)-one | ¹H NMR (DMSO-d6) δ: 8.70 (d, J = 1.8 |
| | | Hz, 1H), 8.64 (s, 1H), 8.44 (d, J = 8.5 Hz, |
| | 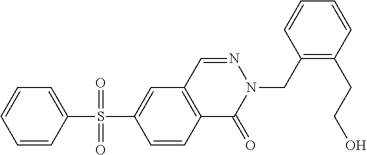 [SMILES: O=c1c2ccc(S(=O)(=O)c3ccccc3)cc2cnn1Cc1ccccc1CCO] | 1H), 8.32 (dd, J = 8.4, 1.9 Hz, 1H), 8.08 − 8.00 (m, 2H), 7.79 − 7.71 (m, 1H), 7.71 − 7.58 (m, 2H), 7.25 − 7.15 (m, 3H), 7.08 (ddd, J = 7.4, 7.1, 1.7 Hz, 1H), 7.01 (d, J = 7.7 Hz, 1H), 5.40 (s, 2H), 4.67 (t, J = 5.2, 5.2 Hz, 1H), 3.65 − 3.49 (m, 2H), 2.90 (t, J = 7.0, 7.0 Hz, 2H). |
| 64 | 2-((2-methyl-2,3-dihydrobenzofuran- | MS (ES⁺): 433 (M + H)⁺ |
| | 5-yl)methyl)-6- | ¹H NMR (400 MHz, DMSO-d6) δ 8.68 (d, |
| | (phenylsulfonyl)phthalazin-1(2H)-one | J = 1.8 Hz, 1H), 8.61 (s, 1H), 8.43 (d, J = |
| | | 8.4 Hz, 1H), 8.30 (dd, J = 8.4, 1.9 Hz, |
| | 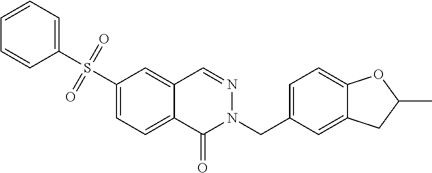 [SMILES: CC1Cc2cc(Cn3ncc4cc(S(=O)(=O)c5ccccc5)ccc4c3=O)ccc2O1] | 1H), 8.06 − 7.99 (m, 2H), 7.78 − 7.70 (m, 1H), 7.70 − 7.62 (m, 2H), 7.16 (d, J = 1.8 Hz, 1H), 7.07 (d, J = 8.1 Hz, 1H), 6.63 (d, J = 8.2 Hz, 1H), 5.21 (s, 2H), 4.90 − 4.79 (m, 1H), 3.23 (dd, J = 15.8, 8.8 Hz, 1H), 2.70 (dd, J = 15.8, 7.6 Hz, 1H), 1.32 (d, J = 6.2 Hz, 3H). |
| 65 | 6-(phenylsulfonyl)-2-(pyridin-3- | MS (ES⁺): 378 (M + H)⁺ |
| | ylmethyl)phthalazin-1(2H)-one | ¹H NMR (DMSO-d6) δ: 8.70 (d, J = 1.9 |
| | | Hz, 1H), 8.64 (s, 1H), 8.58 (dd, J = 2.4, |
| | 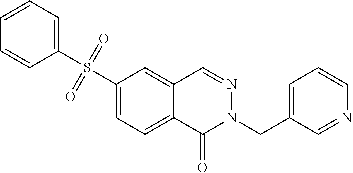 [SMILES: O=c1c2ccc(S(=O)(=O)c3ccccc3)cc2cnn1Cc1cccnc1] | 0.9 Hz, 1H), 8.48 (dd, J = 4.8, 1.7 Hz, 1H), 8.43 (d, J = 8.5, 1H), 8.32 (dd, J = 8.5, 1.9 Hz, 1H), 8.05 − 7.99 (m, 2H), 7.77 − 7.62 (m, 4H), 7.33 (dd, J = 7.9, 4.8, 0.9 Hz, 1H), 5.36 (s, 2H). |
| 66 | 2-((1-oxo-6- | MS (ES⁺): 420 (M + H)⁺ |
| | (phenylsulfonyl)phthalazin-2(1H)- | ¹H NMR (400 MHz, DMSO-d6) δ 8.72 (d, |
| | yl)methyl)benzamide | J = 1.9 Hz, 1H), 8.64 (d, J = 0.6 Hz, 1H), |
| | | 8.44 (d, J = 8.4 Hz, 1H), 8.33 (dd, J = |
| | 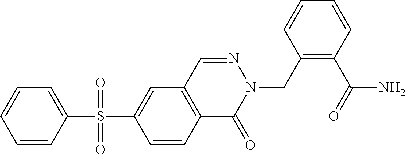 [SMILES: NC(=O)c1ccccc1Cn1ncc2cc(S(=O)(=O)c3ccccc3)ccc2c1=O] | 8.4, 1.9 Hz, 1H), 8.08 − 8.01 (m, 2H), 7.93 (s, 1H), 7.79 − 7.71 (m, 1H), 7.71 − 7.63 (m, 2H), 7.56 − 7.47 (m, 1H), 7.48 (s, 1H), 7.34 − 7.24 (m, 2H), 7.00 − 6.93 (m, 1H), 5.54 (s, 2H). |
| 67 | 2-(2-methoxy-1-phenylethyl)-6- | MS (ES⁺): 421 (M + H)⁺ |
| | (phenylsulfonyl)phthalazin-1(2H)-one | ¹H NMR (DMSO-d6) δ: 8.71 (s, 1H), 8.70 |
| | | (d, J = 1.9 Hz, 1H), 8.42 (d, J = 8.4 Hz, |
| | 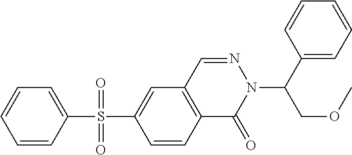 [SMILES: COCC(c1ccccc1)n1ncc2cc(S(=O)(=O)c3ccccc3)ccc2c1=O] | 1H), 8.30 (dd, J = 8.4, 1.8 Hz, 1H), 8.05 − 7.99 (m, 2H), 7.78 − 7.71 (m, 1H), 7.70 − 7.61 (m, 2H), 7.41 − 7.36 (m, 2H), 7.36 − 7.26 (m, 3H), 6.32 (dd, J = 9.7, 5.0 Hz, 1H), 4.24 (t, J = 9.9, 9.9 Hz, 1H), 3.87 (dd, J = 10.1, 5.1 Hz, 1H), 3.26 (s, 3H). |
| 68 | 2-((1H-pyrazol-3-yl)methyl)-6- | MS (ES⁺): 367 (M + H)⁺ |
| | (phenylsulfonyl)phthalazin-1(2H)-one | ¹H NMR (400 MHz, DMSO-d6) δ 12.65 |
| | | (br s, 1H), 8.69 (d, J = 1.8 Hz, 1H), 8.60 |
| | 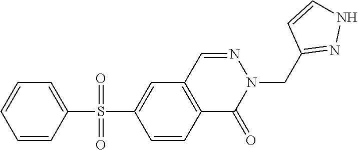 [SMILES: O=c1c2ccc(S(=O)(=O)c3ccccc3)cc2cnn1Cc1cc[nH]n1] | (s, 1H), 8.44 (d, J = 8.4 Hz, 1H), 8.30 (dd, J = 8.4, 1.9 Hz, 1H), 8.07 − 8.00 (m, 2H), 7.78 − 7.71 (m, 1H), 7.70 − 7.63 (m, 2H), 7.62 (d, J = 2.1 Hz, 1H), 6.16 − 6.09 (m, 1H), 5.29 (s, 2H). |
| 69 | 6-(phenylsulfonyl)-2-(1-(pyridin-3- | MS (ES⁺): 409 (M + H)⁺ |
| | yl)ethyl)phthalazin-1(2H)-one | ¹H NMR (DMSO-d6) δ: 8.68 (d, J = 1.8 |
| | | Hz, 1H), 8.67 (s, 1H), 8.58 (d, J = 2.4 Hz, |
| | 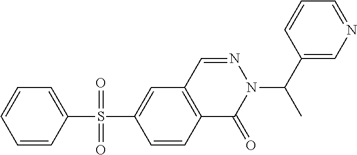 [SMILES: CC(c1cccnc1)n1ncc2cc(S(=O)(=O)c3ccccc3)ccc2c1=O] | 1H), 8.46 (dd, J = 4.8, 1.6 Hz, 1H), 8.43 (d, J = 8.4 Hz, 1H), 8.30 (dd, J = 8.4, 1.9 Hz, 1H), 8.05 − 7.99 (m, 2H), 7.78 − 7.71 (m, 2H), 7.70 − 7.62 (m, 2H), 7.34 (ddd, J = 7.9, 4.8, 0.9 Hz, 1H), 6.31 (q, J = 7.0, 7.0, 7.0 Hz, 1H), 1.75 (d, J = 7.0 Hz, 3H). |
| 70 | 2-((1,3-dimethyl-1H-pyrazol-4- | MS (ES⁺): 395 (M + H)⁺ |
| | yl)methyl)-6- | ¹H NMR (DMSO-d6) δ: 8.66 (d, J = 1.8 |
| | (phenylsulfonyl)phthalazin-1(2H)-one | Hz, 1H), 8.59 (s, 1H), 8.42 (d, J = 8.5 Hz, |
| | | 1H), 8.28 (dd, J = 8.4, 1.9 Hz, 1H), 8.05 − |
| | 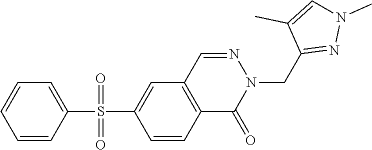 [SMILES: Cc1cn(C)nc1Cn1ncc2cc(S(=O)(=O)c3ccccc3)ccc2c1=O] | 7.96 (m, 2H), 7.77 − 7.68 (m, 1H), 7.69 − 7.57 (m, 2H), 7.30 (s, 1H), 5.10 (s, 2H), 3.65 (s, 3H), 2.29 (s, 3H). MS (ES+): 377 (M + H)+ |
| 71 | 2-benzyl-6- | MS (ES⁺): 377 (M + H)⁺ |
| | (phenylsulfonyl)phthalazin-1(2H)-one | ¹H NMR (DMSO-d6) δ: 8.70 (d, J = 1.8 |
| | | Hz, 1H), 8.64 (s, 1H), 8.44 (d, J = 8.4 Hz, |
| | 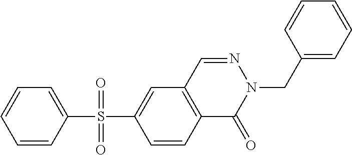 [SMILES: O=c1c2ccc(S(=O)(=O)c3ccccc3)cc2cnn1Cc1ccccc1] | 1H), 8.31 (dd, J = 8.4, 1.9 Hz, 1H), 8.06 − 7.99 (m, 2H), 7.78 − 7.71 (m, 1H), 7.70 − 7.62 (m, 2H), 7.34 − 7.21 (m, 5H), 5.33 (s, 2H). |
| 72 | 6-((4-methoxyphenyl)sulfonyl)-2-((1- | MS (ES⁺): 411 (M + H)⁺ |
| | methyl-1H-pyrazol-3- | ¹H NMR (400 MHz, DMSO-d6) δ 8.62 (d, |
| | yl)methyl)phthalazin-1(2H)-one | J = 1.8 Hz, 1H), 8.58 (d, J = 0.6 Hz, 1H), |
| | | 8.41 (d, J = 8.4 Hz, 1H), 8.26 (dd, J = |
| | 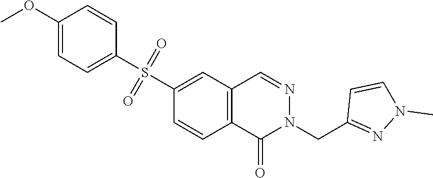 | 8.4, 1.9 Hz, 1H), 7.99 − 7.92 (m, 2H), 7.56 (d, J = 2.2 Hz, 1H), 7.20 − 7.13 (m, 2H), 6.09 (d, J = 2.2 Hz, 1H), 5.24 (s, 2H), 3.83 (s, 3H), 3.75 (s, 3H). |
| 73 | 2-((1-cyclopropyl-1H-pyrazol-3- | MS (ES⁺): 407 (M + H)⁺ |
| | yl)methyl)-6- | ¹H NMR (400 MHz, DMSO-d6) δ 8.68 (d, |
| | (phenylsulfonyl)phthalazin-1(2H)-one | J = 1.8 Hz, 1H), 8.60 (d, J = 0.7 Hz, 1H), |
| | | 8.43 (d, J = 8.4 Hz, 1H), 8.31 (dd, J = |
| | 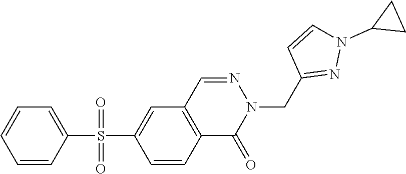 [SMILES: O=c1c2ccc(S(=O)(=O)c3ccccc3)cc2cnn1Cc1ccn(C2CC2)n1] | 8.5, 1.9 Hz, 1H), 8.06 − 7.98 (m, 2H), 7.78 − 7.70 (m, 1H), 7.70 − 7.61 (m, 3H), 6.06 (d, J = 2.3 Hz, 1H), 5.23 (s, 2H), 3.61 (tt, J = 7.4, 3.6 Hz, 1H), 1.00 − 0.93 (m, 2H), 0.92 − 0.85 (m, 2H). |
| 74 | 2-(1-(6-methoxypyridin-3-yl)ethyl)-6- | MS (ES⁺): 422 (M + H)⁺ |
| | (phenylsulfonyl)phthalazin-1(2H)-one | ¹H NMR (DMSO-d6) δ: 8.67 (d, J = 1.8 |
| | | Hz, 1H), 8.65 (s, 1H), 8.43 (d, J = 8.5 Hz, |
| | 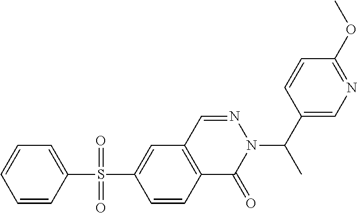 [SMILES: COc1ccc(C(C)n2ncc3cc(S(=O)(=O)c4ccccc4)ccc3c2=O)cn1] | 1H), 8.29 (dd, J = 8.5, 1.9 Hz, 1H), 8.17 (d, J = 2.5 Hz, 1H), 8.05 − 7.98 (m, 2H), 7.77 − 7.63 (m, 4H), 6.75 (d, J = 8.6 Hz, 1H), 6.26 (q, J = 7.0 Hz, 1H), 3.80 (s, 3H), 1.71 (d, J = 7.0 Hz, 3H). |
| 75 | 2-((6-methylpyridin-2-yl)methyl)-6- | MS (ES⁺): 392.0 (M + H)⁺ |
| | (phenylsulfonyl)phthalazin-1(2H)-one | ¹H NMR (400 MHz, DMSO-d6) δ: 8.73 |
| | | (d, J = 1.6 Hz, 1H), 8.65 (s, 1H), 8.44 |
| | 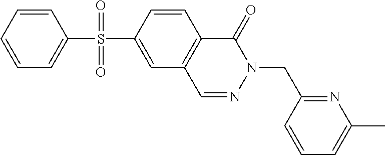 [SMILES: Cc1cccc(Cn2ncc3cc(S(=O)(=O)c4ccccc4)ccc3c2=O)n1] | (d, J = 8.8 Hz, 1H), 8.33 (dd, J = 8.4, 1.6 Hz, 1H), 8.06 (s, 1H), 8.04 (d, J = 1.2 Hz, 1H), 7.76 (t, J = 6.8 Hz, 1H), 7.68 (t, J = 7.6 Hz, 2H), 7.58 (t, J = 7.6 Hz, 1H), 7.12 (d, J = 7.6 Hz, 1H), 6.94 (d, J = 7.6 Hz, 1H), 5.38 (s, 2H), 2.40 (s, 3H). |
| 76 | 2-((4-chloro-1-methyl-1H-pyrazol-3- | MS (ES⁺): 415/417 (M + H)⁺ |
| | yl)methyl)-6- | ¹H NMR (DMSO-d6) δ: 8.68 (d, J = 1.7 |
| | (phenylsulfonyl)phthalazin-1(2H)-one | Hz, 1H), 8.58 (s, 1H), 8.43 (d, J = 8.4 Hz, |
| | | 1H), 8.31 (dd, J = 8.4, 1.9 Hz, 1H), 8.07 − |
| | 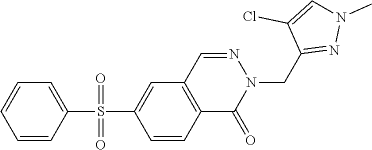 [SMILES: Cn1cc(Cl)c(Cn2ncc3cc(S(=O)(=O)c4ccccc4)ccc3c2=O)n1] | 8.00 (m, 2H), 7.88 (s, 1H), 7.78 − 7.72 (m, 1H), 7.71 − 7.62 (m, 2H), 5.27 (s, 2H), 3.72 (s, 3H). |
| 77 | 2-((2-methylthiazol-4-yl)methyl)-6- | MS (ES⁺): 398 (M + H)⁺ |
| | (phenylsulfonyl)phthalazin-1(2H)-one | ¹H NMR (DMSO-d6) δ: 8.70 (d, J = 1.8 |
| | | Hz, 1H), 8.62 (s, 1H), 8.44 (d, J = 8.4 Hz, |
| | 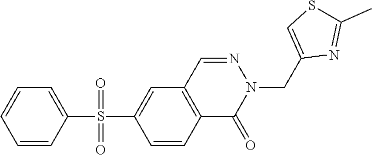 [SMILES: Cc1nc(Cn2ncc3cc(S(=O)(=O)c4ccccc4)ccc3c2=O)cs1] | 1H), 8.32 (dd, J = 8.4, 1.9 Hz, 1H), 8.08 − 8.00 (m, 2H), 7.77 − 7.71 (m, 1H), 7.70 − 7.64 (m, 2H), 7.26 (s, 1H), 5.35 (d, J = 1.0 Hz, 2H), 2.58 (s, 3H). |
| 78 | 2-(difluoro(pyridin-3-yl)methyl)-6- | MS (ES⁺): 414 (M + H)⁺ |
| | (phenylsulfonyl)phthalazin-1(2H)-one | ¹H NMR (400 MHz, DMSO-d6) δ 8.84 (d, |
| | | J = 2.4 Hz, 1H), 8.78 − 8.70 (m, 2H), 8.67 |
| | 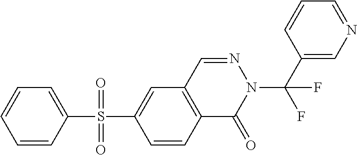 [SMILES: O=c1c2ccc(S(=O)(=O)c3ccccc3)cc2cnn1C(F)(F)c1cccnc1] | (s, 1H), 8.40 − 8.32 (m, 2H), 8.04 (tt, J = 7.1, 1.8 Hz, 3H), 7.79 − 7.72 (m, 1H), 7.67 (tt, J = 6.7, 1.6 Hz, 2H), 7.55 (dd, J = 8.1, 4.8 Hz, 1H). |
| 79 | 2-(2-methoxybenzyl)-6- | MS (ES⁺): 407.0 (M + H)⁺ |
| | (phenylsulfonyl)phthalazin-1(2H)-one | ¹H NMR (400 MHz, DMSO-d6) δ: 8.71 |
| | | (d, J = 1.2 Hz, 1H), 8.63 (s, 1H), 8.48 |
| | 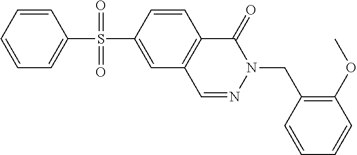 [SMILES: COc1ccccc1Cn1ncc2cc(S(=O)(=O)c3ccccc3)ccc2c1=O] | (d, J = 8.4 Hz, 1H), 8.33 (dd, J = 8.4, 2.0 Hz, 1H), 8.06 (s, 1H), 8.04 (t, J = 2.0 Hz, 1H), 7.77-7.73 (m, 1H), 7.67 (td, J = 8.0, 2.4 Hz, 2H), 7.25 (td, J = 8.8, 1.6 Hz, 1H), 7.01 (d, J = 8.4 Hz, 1H), 8.86 (dd, J = 7.6, 1.6 Hz, 1H), 6.81 (td, J = 8.0, 0.8 Hz, 1H), 5.30 (s, 2H), 3.80 (s, 3H). |
| 80 | 2-((5-methylisoxazol-3-yl)methyl)-6- | MS (ES⁺): 382.0 (M + H)⁺ |
| | (phenylsulfonyl)phthalazin-1(2H)-one | ¹H NMR (400 MHz, DMSO-d6) δ: 8.73 |
| | | (d, J = 1.6 Hz, 1H), 8.65 (s, 1H), 8.45 |
| | 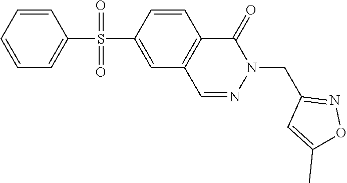 [SMILES: Cc1cc(Cn2ncc3cc(S(=O)(=O)c4ccccc4)ccc3c2=O)no1] | (d, J = 8.4 Hz, 1H), 8.34 (dd, J = 8.4, 2.0 Hz, 1H), 8.06 (s, 1H), 8.04 (d, J = 1.2 Hz, 1H), 7.76 (t, J = 7.2 Hz, 1H), 7.68 (t, J = 8.0 Hz, 2H), 6.15 (s, 1H), 5.34 (s, 2H), 2.34 (s, 3H). |
| 81 | 2-(3-methoxybenzyl)-6- | MS (ES⁺): 406.9 (M + H)⁺ |
| | (phenylsulfonyl)phthalazin-1(2H)-one | ¹H NMR (400 MHz, DMSO-d6) δ: 8.71 |
| | | (d, J = 2.0 Hz, 1H), 8.65 (s, 1H), 8.44 |
| | 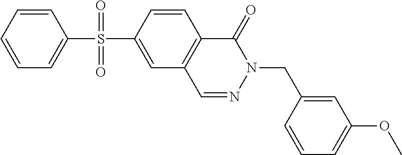 [SMILES: COc1cccc(Cn2ncc3cc(S(=O)(=O)c4ccccc4)ccc3c2=O)c1] | (d, J = 8.4 Hz, 1H), 8.32 (dd, J = 8.4, 1.6 Hz, 1H), 8.05 (s, 1H), 8.04 (t, J = 2.0 Hz, 1H), 7.71-7.73 (m, 1H), 7.69 (s, 1H), 7.67 (dd, J = 7.2, 1.6 Hz, 1H), 7.22 (t, J = 8.0 Hz, 1H), 6.88 (d, J = 2.0 Hz, 1H), 6.85-6.82 (m, 2H), 5.30 (s, 2H), 3.71 (s, 3H). |
| 82 | 2-(2-methoxy-1-phenylethyl)-6-((4- | MS (ES⁺): 451 (M + H)⁺ |
| | methoxyphenyl)sulfonyl)phthalazin- | ¹H NMR (400 MHz, DMSO-d6) δ 8.70 (s, |
| | 1(2H)-one | 1H), 8.64 (d, J = 1.8 Hz, 1H), 8.40 (d, J = |
| | | 8.4 Hz, 1H), 8.26 (dd, J = 8.5, 1.9 Hz, |
| | 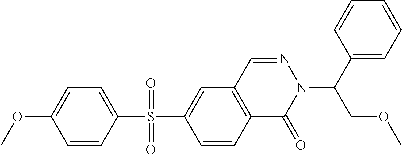 | 1H), 7.97 − 7.91 (m, 2H), 7.41 − 7.35 (m, 2H), 7.35 − 7.24 (m, 3H), 7.19 − 7.12 (m, 2H), 6.32 (dd, J = 9.7, 5.0 Hz, 1H), 4.23 (t, J = 9.9 Hz, 1H), 3.87 (dd, J = 10.1, 5.1 Hz, 1H), 3.83 (s, 3H), 3.26 (s, 3H). |
| 83 | 2-((1-ethyl-1H-pyrazol-5-yl)methyl)-6- | MS (ES⁺): 395 (M + H)⁺ |
| | (phenylsulfonyl)phthalazin-1(2H)-one | ¹H NMR (DMSO-d6) δ: 8.69 (d, J = 1.8 |
| | | Hz, 1H), 8.64 (s, 1H), 8.44 (d, J = 8.4 Hz, |
| | 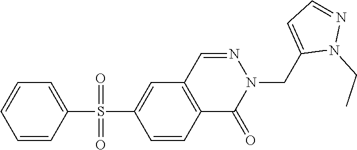 [SMILES: CCn1nccc1Cn1ncc2cc(S(=O)(=O)c3ccccc3)ccc2c1=O] | 1H), 8.32 (dd, J = 8.5, 1.9 Hz, 1H), 8.06 − 7.98 (m, 2H), 7.78 − 7.71 (m, 1H), 7.71 − 7.61 (m, 2H), 7.33 (d, J = 1.8 Hz, 1H), 6.14 (d, J = 1.8 Hz, 1H), 5.40 (s, 2H), 4.23 (q, J = 7.2, 7.2, 7.2 Hz, 2H), 1.28 (t, J = 7.2, 7.2 Hz, 3H). |
| 84 | 3-((1-oxo-6- | MS (ES⁺): 437.0 (M + Na)⁺ |
| | (phenylsulfonyl)phthalazin-2(1H)- | ¹H NMR (400 MHz, DMSO-d6) δ: 8.71 |
| | yl)methyl)benzamide | (d, J = 1.2 Hz, 1H), 8.65 (s, 1H), 8.44(d, |
| | | J = 7.6 Hz, 1H), 8.32(dd, J₁ = 1.6 Hz, J₂ = |
| | 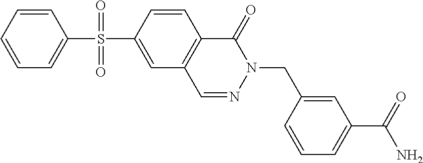 [SMILES: NC(=O)c1cccc(Cn2ncc3cc(S(=O)(=O)c4ccccc4)ccc3c2=O)c1] | 8.4 Hz, 1H), 8.03 (d, J = 8.0 Hz, 1H), 7.95(s, 1H), 7.78-7.72 (m, 3H), 7.67 (t, J = 7.2 Hz, 2H),7.45 (d, J = 3.6 Hz, 1H), 7.41-7.34 (m, 2H), 5.37 (s, 2H). |
| 85 | 3-((1-oxo-6- | MS (ES⁺): 410.0 (M + H)⁺ |
| | (phenylsulfonyl)phthalazin-2(1H)- | ¹H NMR (400 MHz, DMSO-d6) δ: 8.71 |
| | yl)methyl)furan-2-carboxamide O=S=δ N | (d, J = 1.6 Hz, 1H), 8.63 (s, 1H), 8.44(d, |
| | | J = 8.4 Hz, 1H), 8.33(dd, J₁ = 2.0 Hz, J₂ = |
| | 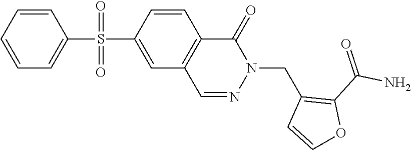 [SMILES: NC(=O)c1occc1Cn1ncc2cc(S(=O)(=O)c3ccccc3)ccc2c1=O] | 8.4 Hz, 1H), 8.04 (d, J = 8.0 Hz, 2H), 7.79-7.73 (m, 2H), 7.70-7.65 (m, 3H), 7.51 (s, 1H), 6.33 (d, J = 1.6 Hz, 1H), 5.54 (s, 2H). |
| 86 | 5-((1-oxo-6- | MS (ES⁺): 409.9 (M + H)⁺ |
| | (phenylsulfonyl)phthalazin-2(1H)- | ¹H NMR (400 MHz, DMSO-d6) δ: 8.71 |
| | yl)methyl)furan −2-carboxamide | (d, J = 1.2 Hz, 1H), 8.64 (s, 1H), 8.44 (d, |
| | | J = 8.4 Hz, 1H), 8.33 (dd, J₁ = 1.6 Hz, |
| | 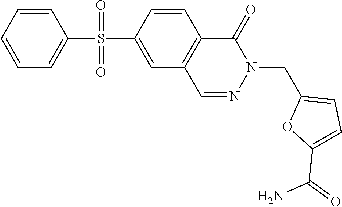 [SMILES: NC(=O)c1ccc(Cn2ncc3cc(S(=O)(=O)c4ccccc4)ccc3c2=O)o1] | J = 8.4 Hz, 1H), 8.03 (d, J = 8.0 Hz, 2H), 7.75-7.72 (m, 1H), 7.69-7.65 (m, 3H), 7.32(s, 1H), 7.04 (d, J = 3.6 Hz, 1H), 6.48 (d, J = 3.6 Hz, 1H), 5.36 (s, 2H). |
| 87 | 2-((5-methoxypyrazin-2-yl)methyl)-6- | MS (ES⁺): 409.0 (M + H)⁺ |
| | (phenylsulfonyl)phthalazin-1(2H)-one | ¹H NMR (400 MHz, DMSO-d6) δ: 8.70 |
| | | (d, J = 1.6 Hz, 1H), 8.62 (s, 1H), 8.42 (d, |
| | 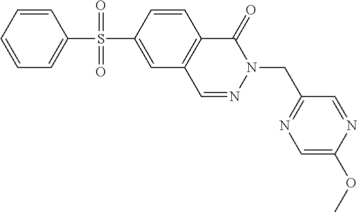 [SMILES: COc1cnc(Cn2ncc3cc(S(=O)(=O)c4ccccc4)ccc3c2=O)cn1] | J = 8.4 Hz, 1H), 8.32 (dd, J = 8.4, 2.0 Hz, 1H), 8.24 (d, J = 1.2 Hz, 1H), 8.20 (d, J = 1.2 Hz, 1H), 8.05 (s, 1H), 8.03 (d, J = 1.2 Hz, 1H), 7.75 (t, J = 7.2 Hz, 1H), 7.67 (t, J = 8.0 Hz, 2H), 5.42 (s, 2H), 3.88 (s, 3H). |
| 88 | 2-((2-methoxypyridin-3-yl)methyl)-6- | MS (ES⁺): 408 (M + H)⁺ |
| | (phenylsulfonyl)phthalazin-1(2H)-one | ¹H NMR (DMSO-d6) δ: 8.71 (d, J = 1.8 |
| | | Hz, 1H), 8.64 (s, 1H), 8.44 (d, J = 8.4 Hz, |
| | 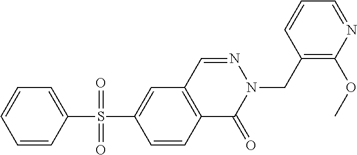 [SMILES: COc1ncccc1Cn1ncc2cc(S(=O)(=O)c3ccccc3)ccc2c1=O] | 1H), 8.33 (dd, J = 8.4, 1.9 Hz, 1H), 8.08 (dd, J = 5.0, 1.9 Hz, 1H), 8.06 − 8.02 (m, 2H), 7.78 − 7.72 (m, 1H), 7.71 − 7.63 (m, 2H), 7.33 (dd, J = 7.3, 1.8 Hz, 1H), 6.88 (dd, J = 7.3, 5.0 Hz, 1H), 5.26 (s, 2H), 3.89 (s, 3H). |
| 89 | 2-((1,4-dimethyl-1H-pyrazol-3- | MS (ES⁺): 395 (M + H)⁺ |
| | yl)methyl)-6- | ¹H NMR (400 MHz, DMSO-d6) δ 8.67 (d, |
| | (phenylsulfonyl)phthalazin-1(2H)-one | J = 1.8 Hz, 1H), 8.58 (s, 1H), 8.43 (d, J = |
| | | 8.5 Hz, 1H), 8.30 (dd, J = 8.4, 1.9 Hz, |
| | 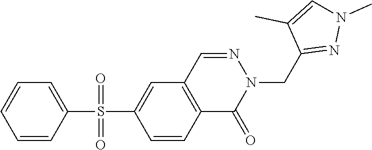 [SMILES: Cc1cn(C)nc1Cn1ncc2cc(S(=O)(=O)c3ccccc3)ccc2c1=O] | 1H), 8.06 − 8.00 (m, 2H), 7.78 − 7.71 (m, 1H), 7.70 − 7.62 (m, 2H), 7.36 (s, 1H), 5.22 (s, 2H), 3.66 (s, 3H), 1.93 (s, 3H). |
| 90 | 2-((1,5-dimethyl-1H-pyrazol-3- | MS (ES⁺): 395 (M + H)⁺ |
| | yl)methyl)-6- | ¹H NMR (DMSO-d6) δ: 8.68 (d, J = 1.7 |
| | (phenylsulfonyl)phthalazin-1(2H)-one | Hz, 1H), 8.59 (d, J = 0.7 Hz, 1H), 8.42 |
| | | (d, J = 8.4 Hz, 1H), 8.30 (dd, J = 8.5, 1.8 |
| | 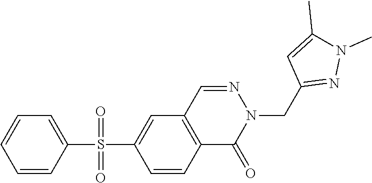 [SMILES: Cc1cc(Cn2ncc3cc(S(=O)(=O)c4ccccc4)ccc3c2=O)nn1C] | Hz, 1H), 8.06 − 7.99 (m, 2H), 7.77 − 7.72 (m, 1H), 7.71 − 7.63 (m, 2H), 5.87 (s, 1H), 5.17 (s, 2H), 3.61 (s, 3H), 2.14 (d, J = 0.7 Hz, 3H). |
| 91 | 2-chloro-6-((1-oxo-6- | MS (ES⁺): 453.9 (M + H)⁺ |
| | (phenylsulfonyl)phthalazin-2(1H)- | ¹H NMR (400 MHz, DMSO-d6) δ: 8.73 |
| | yl)methyl)benzamide | (d, J = 1.6 Hz, 1H), 8.64 (s, 1H), 8.45 |
| | | (d, J = 8.4 Hz, 1H), 8.34 (dd, J = 8.4, |
| | 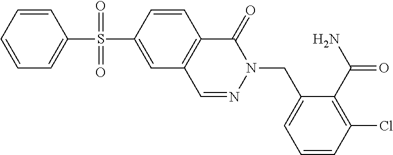 [SMILES: NC(=O)c1c(Cl)cccc1Cn1ncc2cc(S(=O)(=O)c3ccccc3)ccc2c1=O] | 2.0 Hz, 1H), 8.06 (s, 2H), 8.04 (t, J = 2.0 Hz, 1H), 7.79 (br, 1H), 7.74 (t, J = 6.4 Hz, 1H), 7.68 (t, J = 7.6 Hz, 2H), 7.39 (d, J = 7.6 Hz, 1H), 7.25 (t, J = 7.6 Hz, 1H), 6.96 (d, J = 7.6 Hz, 1H), 5.36 (s, 2H). |
| 92 | 2-methoxy-6-((1-oxo-6- | MS (ES⁺): 449.9 (M + H)⁺ |
| | (phenylsulfonyl)phthalazin-2(1H)- | ¹H NMR (400 MHz, DMSO-d6) δ: 8.72 |
| | yl)methyl)benzamide | (d, J = 1.6 Hz, 1H), 8.63 (s, 1H), 8.44 |
| | | (d, J = 8.4 Hz, 1H), 8.33 (dd, J = 8.4, |
| | 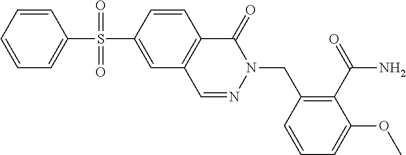 [SMILES: COc1cccc(Cn2ncc3cc(S(=O)(=O)c4ccccc4)ccc3c2=O)c1C(N)=O] | 2.0 Hz, 1H), 8.06 (s, 1H), 8.04 (d, J = 1.2 Hz, 1H), 7.76-7.66 (m, 4H), 7.50 (br, 1H), 7.18 (t, J = 8.0 Hz, 1H), 6.95 (d, J = 8.0 Hz, 1H), 6.59 (d, J = 8.0 Hz, 1H), 5.32 (s, 2H), 3.76 (s, 3H). |
| 93 | 4-((1-oxo-6- | MS (ES⁺): 427.0 (M + H)⁺ |
| | (phenylsulfonyl)phthalazin-2(1H)- | ¹H NMR (400 MHz, DMSO-d6) δ: 8.72 |
| | yl)methyl)thiazole-2-carboxamide | (d, J = 1.6 Hz, 1H), 8.65 (s, 1H), 8.45(d, |
| | | J = 8.4 Hz, 1H), 8.33(dd, J₁ = 2.0 Hz, J₂ = |
| | 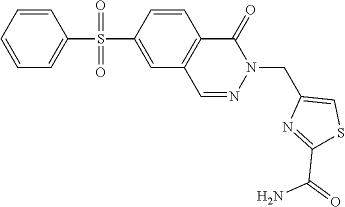 [SMILES: NC(=O)c1nc(Cn2ncc3cc(S(=O)(=O)c4ccccc4)ccc3c2=O)cs1] | 8.4 Hz, 1H), 8.04 (d, J = 8.0 Hz, 3H), 7.82(s, 1H), 7.77-7.73 (m, 2H), 7.70- 7.65 (m,2H), 5.47(s, 2H). |
| 94 | 5-methyl-4-((1-oxo-6- | MS (ES⁺): 424.9 (M + H)⁺ |
| | (phenylsulfonyl)phthalazin-2(1H)- | ¹H NMR (400 MHz, DMSO-d6) δ: 8.68 |
| | yl)methyl) isoxazole-3-carboxamide | (d, J = 1.2 Hz, 1H), 8.56 (s, 1H), 8.42 |
| | | (d, J = 8.8 Hz, 1H), 8.30 (dd, J₁ = 2.0 |
| | 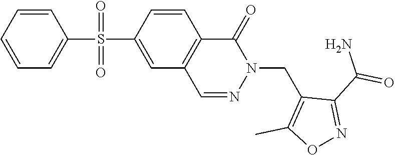 [SMILES: Cc1onc(C(N)=O)c1Cn1ncc2cc(S(=O)(=O)c3ccccc3)ccc2c1=O] | Hz, J2 = 8.4 Hz, 1H), 8.05-8.01 (m, 3H), 7.77-7.72 (m, 2H), 7.69-7.64 (m,2H), 5.34(s, 2H), 2.38(s, 3H). |
| 95 | 2-((1-isopropyl-1H-pyrazol-3- | MS (ES⁺): 409 (M + H)⁺ |
| | yl)methyl)-6- | ¹H NMR (DMSO-d6) δ: 8.68 (d, J = 1.9 |
| | (phenylsulfonyl)phthalazin-1(2H)-one | Hz, 1H), 8.61 (s, 1H), 8.44 (d, J = 8.5 Hz, |
| | | 1H), 8.31 (dd, J = 8.4, 1.9 Hz, 1H), 8.06 − |
| | 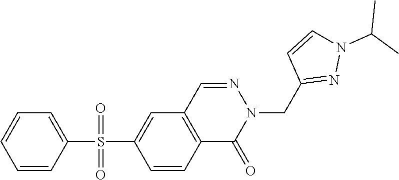 [SMILES: CC(C)n1ccc(Cn2ncc3cc(S(=O)(=O)c4ccccc4)ccc3c2=O)n1] | 8.00 (m, 2H), 7.78 − 7.71 (m, 1H), 7.70 − 7.60 (m, 3H), 6.06 (d, J = 2.3 Hz, 1H), 5.26 (s, 2H), 4.41 (hept, J = 6.7 Hz, 1H), 1.36 (d, J = 6.6 Hz, 6H). |
| 96 | 2-((5-methyl-1H-pyrazol-3-yl)methyl)- | MS (ES⁺): 381 (M + H)⁺ |
| | 6-(phenylsulfonyl)phthalazin-1(2H)- | ¹H NMR (DMSO-d6) δ: 12.29 (s, 1H), |
| | one | 8.68 (d, J = 1.8 Hz, 1H), 8.59 (s, 1H), |
| | | 8.43 (d, J = 8.5 Hz, 1H), 8.30 (dd, J = |
| | 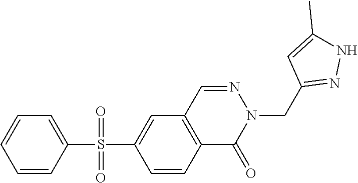 [SMILES: Cc1cc(Cn2ncc3cc(S(=O)(=O)c4ccccc4)ccc3c2=O)n[nH]1] | 8.4, 1.9 Hz, 1H), 8.06 − 7.99 (m, 2H), 7.79 − 7.70 (m, 1H), 7.70 − 7.62 (m, 2H), 5.84 (s, 1H), 5.21 (s, 2H), 2.13 (s, 3H). |
| 97 | 2-((3-methylisothiazol-5-yl)methyl)-6- | MS (ES⁺): 398 (M + H)⁺ |
| | (phenylsulfonyl)phthalazin-1(2H)-one | ¹H NMR (DMSO-d6) δ: 8.71 (d, J = 1.8 |
| | | Hz, 1H), 8.67 (s, 1H), 8.45 (d, J = 8.4 Hz, |
| | 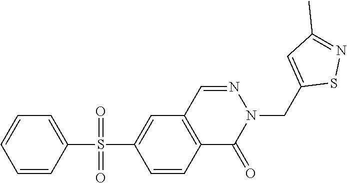 [SMILES: Cc1cc(Cn2ncc3cc(S(=O)(=O)c4ccccc4)ccc3c2=O)sn1] | 1H), 8.33 (dd, J = 8.4, 1.9 Hz, 1H), 8.07 − 7.99 (m, 2H), 7.79 − 7.70 (m, 1H), 7.70 − 7.62 (m, 2H), 7.17 (s, 1H), 5.57 (s, 2H), 2.34 (s, 3H). |
| 98 | 2-((2-hydroxy-6-methylpyridin-3- | MS (ES⁺): 438.0 (M + H)⁺ |
| | yl)methyl)-6-(4- | ¹H NMR (400 MHz, DMSO-d6) δ: 11.67 |
| | methoxyphenylsulfonyl)phthalazin- | (br, 1H), 8.65 (d, J = 1.2 Hz, 1H), 8.61 |
| | 1(2H)-one | (s, 1H), 8.42 (d, J = 8.4 Hz 1H), 8.28 |
| | | (dd, J = 8.4, 1.6 Hz, 1H), 7.97 (d, J = |
| | 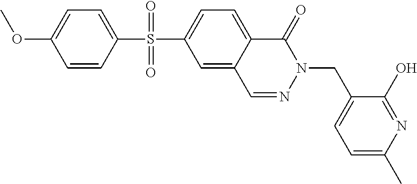 | 11.6 Hz, 2H), 7.18 (d, J = 12 Hz, 2H), 6.93 (d, J = 6.8 Hz, 1H), 5.87 (d, J = 7.2 Hz, 1H) 5.04 (s, 2H), 3.84 (s, 3H), 2.13 (s, 3H). |
| 99 | 2-((2-hydroxy-4-methylpyridin-3- | MS (ES⁺): 438.0 (M + H)⁺ |
| | yl)methyl)-6-(4- | ¹H NMR (400 MHz, DMSO-d6) δ: 11.43 |
| | methoxyphenylsulfonyl)phthalazin- | (br, 1H), 8.58 (d, J = 1.6 Hz, 1H), 8.49 |
| | 1(2H)-one | (s, 1H), 8.42 (d, J = 8.4 Hz 1H), 8.25 |
| | | (dd, J = 8.8, 1.6 Hz, 1H), 7.97-7.94 (m, |
| | 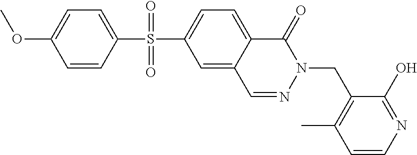 [SMILES: COc1ccc(S(=O)(=O)c2ccc3c(=O)n(Cc4c(C)ccnc4O)ncc3c2)cc1] | 2H), 7.23 (d, J = 6.8 Hz, 1H), 7.16 (dd, J = 7.2, 2.0 Hz, 2H), 6.05 (d, J = 6.8 Hz, 1H), 5.18 (s, 2H), 3.83 (s, 3H), 2.11 (s, 3H). |
| 100 | 2-((5-fluoro-2-hydroxypyridin-3- | MS (ES⁺): 442.0 (M + H)⁺ |
| | yl)methyl)-6-(4- | ¹H NMR (400 MHz, DMSO-d6) δ: 11.64 |
| | methoxyphenylsulfonyl)phthalazin- | (br, 1H), 8.65 (d, J = 1.6 Hz, 1H), 8.62 |
| | 1(2H)-one | (s, 1H), 8.42 (d, J = 8.4 Hz 1H), 8.29 (dd, |
| | | J = 8.8, 2.0 Hz, 1H), 8.00-7.95 (m, 2H), |
| | 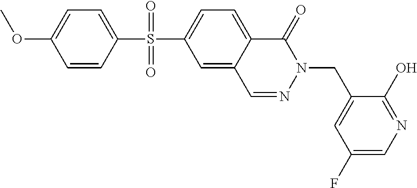 [SMILES: COc1ccc(S(=O)(=O)c2ccc3c(=O)n(Cc4cc(F)cnc4O)ncc3c2)cc1] | 7.53 (t, J = 3.2 Hz, 1H), 7.24 (d, J = 8.0, 3.2 Hz, 1H), 7.20-7.16 (m, 2H), 5.08 (s, 2H), 3.84 (s, 3H). |
| 101 | 2-((2-hydroxy-5-isopropylpyridin-3- | MS (ES⁺): 466.0 (M + H)⁺ |
| | yl)methyl)-6-(4- | ¹H NMR (400 MHz, DMSO-d6) δ: 11.53 |
| | methoxyphenylsulfonyl)phthalazin- | (br, 1H), 8.65 (d, J = 1.6 Hz, 1H), 8.61 |
| | 1(2H)-one | (s, 1H), 8.43 (d, J = 8.8 Hz 1H), 8.29 (dd, |
| | | J = 8.4, 1.6 Hz, 1H), 7.99-7.96 (m, 2H), |
| | 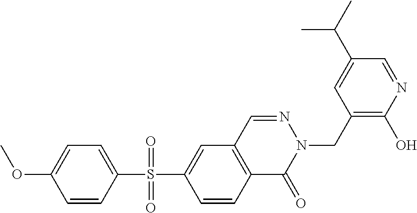 [SMILES: COc1ccc(S(=O)(=O)c2ccc3c(=O)n(Cc4cc(C(C)C)cnc4O)ncc3c2)cc1] | 7.20-7.17 (m, 2H), 7.07 (s, 1H), 7.06 (d, J = 2.0 Hz, 1H), 5.08 (s, 2H), 3.84 (s, 3H), 2.61-2.54 (m, 1H), 1.02 (s, 3H), 1.00 (s, 3H). |
| 102 | 2-((4-chloro-2-hydroxypyridin-3- | MS (ES⁺): 457.9 (M + H)⁺ |
| | yl)methyl)-6-(4- | ¹H NMR (400 MHz, CD₃OD) δ: 11.93 |
| | methoxyphenylsulfonyl) phthalazin- | (br, 1H), 8.59 (d, J = 1.6 Hz, 1H), 8.50 |
| | 1(2H)-one | (s, 1H), 8.43 (d, J = 8.4 Hz, 1H), 8.27 |
| | | (dd, J = 8.4, 1.6 Hz, 1H), 7.97-7.94 (m, |
| | 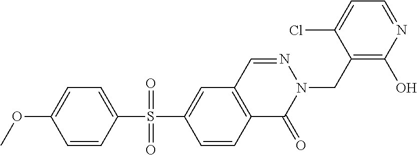 [SMILES: COc1ccc(S(=O)(=O)c2ccc3c(=O)n(Cc4c(Cl)ccnc4O)ncc3c2)cc1] | 2H), 7.43 (d, J = 6.8 Hz, 1H), 7.18-7.16 (m, 2H), 6.32 (d, J = 6.8 Hz, 1H), 5.25 (s, 2H), 3.83 (s, 3H). |
| 103 | 2-((4-fluoro-2-hydroxypyridin-3- | MS (ES⁺): 441.9 (M + H)⁺ |
| | yl)methyl)-6-(4- | ¹H NMR (400 MHz, DMSO-d6) δ: 11.86 |
| | methoxyphenylsulfonyl) phthalazin- | (br, 1H), 8.60 (d, J = 1.6 Hz, 1H), 8.52 |
| | 1(2H)-one | (s, 1H), 8.42 (d, J = 2.0 Hz, 1H), 8.26 |
| | | (dd, J = 8.4, 1.6 Hz, 1H), 7.95 (d, J = |
| | 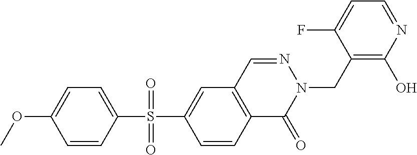 [SMILES: COc1ccc(S(=O)(=O)c2ccc3c(=O)n(Cc4c(F)ccnc4O)ncc3c2)cc1] | 9.2 Hz, 2H), 7.50 (t, J = 7.2 Hz, 1H), 7.16 (d, J = 9.2 Hz, 2H), 6.23 (t, J = 7.6 Hz, 1H), 5.14 (s, 2H), 3.83 (s, 3H). |
| 104 | 6-((4-methoxyphenyl)sulfonyl)-2-((4- | MS (ES⁺): 411 (M + H)⁺ |
| | methyl-1H-pyrazol-3- | ¹H NMR (400 MHz, DMSO-d6) δ 12.35 |
| | yl)methyl)phthalazin-1(2H)-one | (s, 1H), 8.62 (s, 1H), 8.58 (d, J = 9.8 Hz, |
| | | 1H), 8.42 (d, J = 8.4 Hz, 1H), 8.26 (dd, |
| | 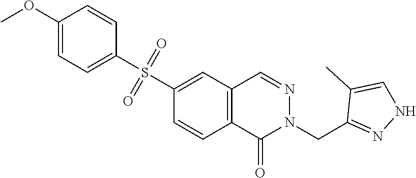 [SMILES: COc1ccc(S(=O)(=O)c2ccc3c(=O)n(Cc4n[nH]cc4C)ncc3c2)cc1] | J = 8.4, 1.8 Hz, 1H), 7.99 − 7.92 (m, 2H), 7.41 (s, 1H), 7.19 − 7.13 (m, 2H), 5.26 (d, J = 5.4 Hz, 2H), 3.83 (s, 3H), 1.95 (d, J = 2.5 Hz, 3H). |
| 105 | 2-((3-hydroxypyridin-4-yl)methyl)-6- | MS (ES⁺): 424.0 (M + H)⁺ |
| | (4- | ¹H NMR (400 MHz, DMSO-d6) δ: 10.14 |
| | methoxyphenylsulfonyl)phthalazin- | (br, 1H), 8.67 (d, J = 1.6 Hz, 1H), 8.65 |
| | 1(2H)-one | (s, 1H), 8.43 (d, J = 8.4 Hz 1H), 8.30 |
| | | (dd, J = 8.4, 2.0 Hz, 1H), 8.14 (s, 1H), |
| | 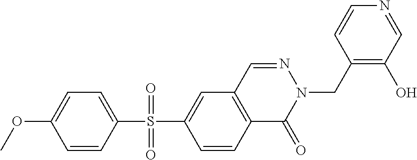 [SMILES: COc1ccc(S(=O)(=O)c2ccc3c(=O)n(Cc4ccncc4O)ncc3c2)cc1] | 7.97 (dd, J = 7.2, 2.0 Hz, 2H), 7.89 (d, J = 4.8 Hz, 1H) 7.18 (dd, J = 7.2, 2.0 Hz, 2H), 6.79 (d, J = 4.8 Hz, 1H), 5.28 (s, 2H), 3.84 (s, 3H). |
| 106 | 5-methoxy-2-((6-(4- | MS (ES⁺): 479.9 (M + H)⁺ |
| | methoxyphenylsulfonyl)-1- | ¹H NMR (400 MHz, DMSO-d6) δ: 8.66 |
| | oxophthalazin-2(1H)- | (d, J = 1.2 Hz, 1H), 8.61 (s, 1H), 8.41 |
| | yl)methyl)benzamide | (d, J = 8.4 Hz, 1H), 8.28 (dd, J = 8.4, |
| | | 1.6 Hz, 1H), 7.96 (d, J = 9.2 Hz, 2H), |
| | 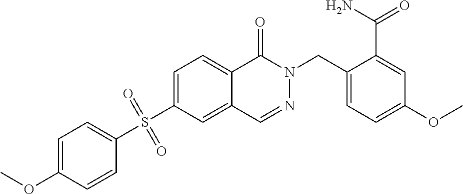 | 7.93 (br, 1H), 7.49 (br, 1H), 7.18 (d, J = 9.2 Hz, 2H), 7.06 (d, J = 2.4 Hz, 1H), 6.95 (d, J = 8.4 Hz, 1H), 6.87 (d, J = 8.8, 2.8 Hz, 1H), 5.47 (s, 2H), 3.84 (s, 3H), 3.75 (s, 3H). |
| 107 | 2-((4-hydroxypyridin-3-yl)methyl)-6- | MS (ES⁺): 424.0 (M + H)⁺ |
| | (4- | ¹H NMR (400 MHz, DMSO-d6) δ: 8.64 |
| | methoxyphenylsulfonyl)phthalazin- | (d, J = 1.6 Hz, 1H), 8.59 (s, 1H), 8.43 |
| | 1(2H)-one | (d, J = 8.4 Hz 1H), 8.28 (dd, J = 8.4, 1.6 |
| | | Hz, 1H), 8.14 (s, 1H), 7.97 (dd, J = 7.2, |
| | 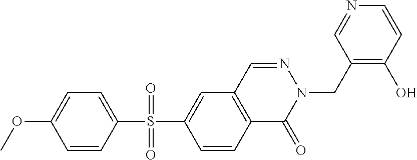 [SMILES: COc1ccc(S(=O)(=O)c2ccc3c(=O)n(Cc4cnccc4O)ncc3c2)cc1] | 2.0 Hz, 2H), 7.64 (d, J = 6.8 Hz, 1H), 7.42 (br, 1H), 7.18 (d, J = 9.2 Hz, 2H), 6.14 (d, J = 5.2 Hz, 1H), 5.05 (s, 2H), 3.84 (s, 3H). |
| 108 | 6-(phenylsulfonyl)-2-(pyrazolo[1,5- | MS (ES⁺): 417 (M + H)⁺ |
| | a]pyridin-2-ylmethyl)phthalazin- | ¹H NMR (DMSO-d6) δ: 8.71 (d, J = 1.8 |
| | 1(2H)-one | Hz, 1H), 8.63 (s, 1H), 8.61 − 8.54 (m, |
| | | 1H), 8.45 (d, J = 8.4 Hz, 1H), 8.32 (dd, |
| | 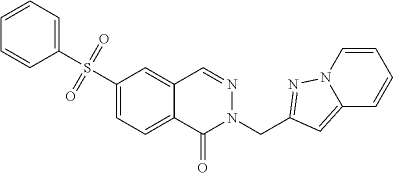 [SMILES: O=c1c2ccc(S(=O)(=O)c3ccccc3)cc2cnn1Cc1cc2ccccn2n1] | J = 8.4, 1.9 Hz, 1H), 8.08 − 8.00 (m, 2H), 7.79 − 7.71 (m, 1H), 7.71 − 7.62 (m, 2H), 7.60 − 7.52 (m, 1H), 7.16 (ddd, J = 8.9, 6.7, 1.1 Hz, 1H), 6.82 (ddd, J = 6.9, 6.8, 1.4 Hz, 1H), 6.44 (s, 1H), 5.50 (s, 2H). |
| 109 | 2-((2-hydroxy-6-methoxypyridin-3- | MS (ES⁺): 454.1 (M + H)⁺ |
| | yl)methyl)-6-(4- | ¹H NMR (400 MHz, DMSO-d6) δ: 11.16 |
| | methoxyphenylsulfonyl)phthalazin- | (br, 1H), 8.63 (d, J = 1.6 Hz, 1H), 8.59 |
| | 1(2H)-one | (s, 1H), 8.41 (d, J = 8.8 Hz, 1H), 8.27 |
| | | (dd, J = 8.4, 1.6 Hz, 1H), 7.97-7.94 (m, |
| | 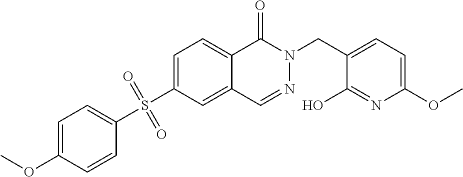 | 2H), 7.27 (d, J = 8.0 Hz, 1H), 7.18-7.15 (m, 2H), 5.99 (br, 1H), 5.13 (s, 2H), 3.84 (s, 3H), 3.76 (s, 3H). |
| 110 | 2-((4-fluoro-1H-pyrazol-3-yl)methyl)- | MS (ES⁺): 385 (M + H)⁺ |
| | 6-(phenylsulfonyl)phthalazin-1(2H)- | ¹H NMR (400 MHz, DMSO-d6) δ 12.40 |
| | one | (s, 1H), 8.61 (s, 1H), 8.55 (d, J = 9.2 Hz, |
| | | 1H), 8.44 (d, J = 8.4 Hz, 1H), 8.27 (d, J = |
| | 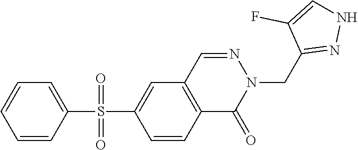 [SMILES: O=c1c2ccc(S(=O)(=O)c3ccccc3)cc2cnn1Cc1n[nH]cc1F] | 7.9 Hz, 1H), 8.06 − 7.95 (m, 2H), 7.77 − 7.69 (m, 1H), 7.69 − 7.57 (m, 3H), 5.34 (s, 2H). |
| 111 | 2-((5-fluoro-1H-pyrazol-3-yl)methyl)- | MS (ES⁺): 385 (M + H)⁺ |
| | 6-(phenylsulfonyl)phthalazin-1(2H)- | ¹H NMR (400 MHz, DMSO-d6) δ 12.45 |
| | one | (s, 1H), 8.70 (d, J = 1.8 Hz, 1H), 8.63 (d, |
| | | J = 0.7 Hz, 1H), 8.44 (d, J = 8.4 Hz, 1H), |
| | 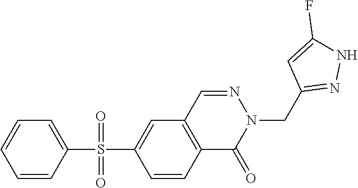 [SMILES: O=c1c2ccc(S(=O)(=O)c3ccccc3)cc2cnn1Cc1cc(F)[nH]n1] | 8.33 (dd, J = 8.5, 1.9 Hz, 1H), 8.07 − 8.00 (m, 2H), 7.79 − 7.71 (m, 1H), 7.70 − 7.62 (m, 2H), 5.86 (d, J = 6.1 Hz, 1H), 5.28 (s, 2H). |
| 112 | 6-(4-methoxyphenylsulfonyl)-2-((3- | MS (ES⁺): 468.0 (M + H)⁺ |
| | (trifluoromethyl)isoxazol-5- | ¹H NMR (400 MHz, DMSO-d6) δ: 8.67 |
| | yl)methyl)phthalazin-1(2H)-one | (d, J = 1.6 Hz, 1H), 8.65 (s, 1H), 8.43 (d, |
| | | J = 8.4 Hz, 1H), 8.30 (dd, J₁ = 1.6 Hz, J₂ = |
| | 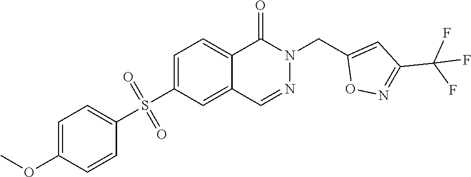 [SMILES: COc1ccc(S(=O)(=O)c2ccc3c(=O)n(Cc4cc(C(F)(F)F)no4)ncc3c2)cc1] | 8.4 Hz, 1H), 7.96 (dd, J₁ = 2.0 Hz, J₂ = 6.8 Hz, 1H, 2H), 7.41 (d, J = 1.2 Hz, 1H), 7.17 (dd, J₁ = 2.0 Hz, J₂ = 6.8 Hz, 1H, 2H), 5.51 (s, 2H), 3.84 (s, 3H). |
| 113 | 2-((3-hydroxypyridin-2-yl)methyl)-6- | MS (ES⁺): 424.0 (M + H)⁺ |
| | (4- | ¹H NMR (400 MHz, DMSO-d6) δ: 10.16 |
| | methoxyphenylsulfonyl)phthalazin- | (br, 1H), 8.65 (d, J = 1.6 Hz, 1H), 8.58 |
| | 1(2H)-one | (s, 1H), 8.40 (d, J = 8.4 Hz 1H), 8.27 (dd, |
| | | J = 8.4, 1.6 Hz, 1H), 8.14 (s, 1H), 7.97 |
| | 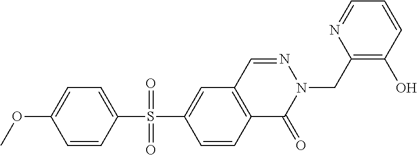 [SMILES: COc1ccc(S(=O)(=O)c2ccc3c(=O)n(Cc4ncccc4O)ncc3c2)cc1] | (d, J = 9.2 Hz, 2H), 7.78 (d, J = 4.8 Hz, 1H), 7.18 (d, J = 8.8 Hz, 2H), 7.09 (dd, J = 8.0, 4.4 Hz, 1H), 5.39 (s, 2H), 3.84 (s, 3H). |

### Example 114—2-(3-aminobenzyl)-6-(quinolin-8-ylsulfonyl)phthalazin-1(2H)-one

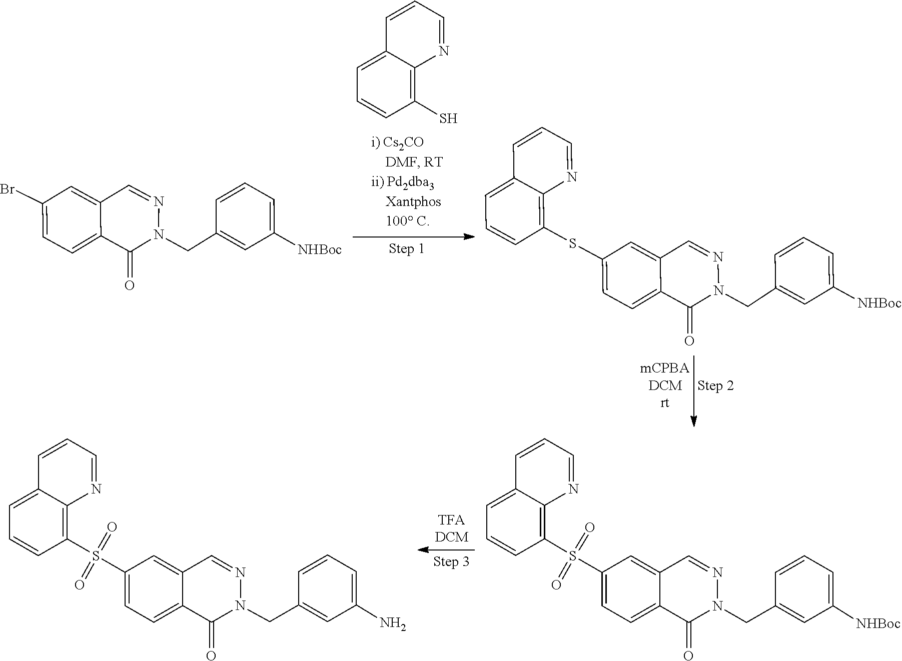 [SMILES: Sc1cccc2cccnc12 ; CC(C)(C)OC(=O)Nc1cccc(Cn2ncc3cc(Br)ccc3c2=O)c1 ; CC(C)(C)OC(=O)Nc1cccc(Cn2ncc3cc(Sc4cccc5cccnc45)ccc3c2=O)c1 ; Nc1cccc(Cn2ncc3cc(S(=O)(=O)c4cccc5cccnc45)ccc3c2=O)c1 ; CC(C)(C)OC(=O)Nc1cccc(Cn2ncc3cc(S(=O)(=O)c4cccc5cccnc45)ccc3c2=O)c1]

### Step 1

N₂ was bubbled through a mixture of tert-butyl (3-((6-bromo-1-oxophthalazin-2(1H)-yl)methyl)phenyl)carbamate (Intermediate 41, 100 mg, 1 Eq), quinoline-8-thiol hydrochloride (45.9 mg, 1 Eq, 232 μmol) and cesium carbonate (227 mg, 3 Eq, 697 μmol) in DMF (1.5 mL) and the mixture was stirred for 5 minutes. Pd₂dba₃ (10.6 mg, 0.05 Eq, 11.6 μmol) and Xantphos (13.4 mg, 0.1 Eq, 23.2 μmol) were added and the mixture was stirred at 100° C. under N₂ for 3 h, then allowed to cool to RT. Water and DCM were added and the layers separated through a phase separating cartridge. The organic layer was washed with brine, then absorbed on silica. The crude product was purified by chromatography on silica gel to afford tert-butyl (3-((1-oxo-6-(quinolin-8-ylthio)phthalazin-2(1H)-yl)methyl)phenyl)carbamate (82 mg) as a pale yellow solid. MS (ES⁺): 511 (M+H)⁺.

### Step 2

mCPBA (99 mg, 70% Wt, 2.5 Eq, 0.40 mmol) was added to a solution of tert-butyl (3-((1-oxo-6-(quinolin-8-ylthio)phthalazin-2(1H)-yl)methyl)phenyl)carbamate (82 mg, 1 Eq, 0.16 mmol) in DCM (1 mL) and the mixture was stirred for 1 h. DCM and 1 N NaOH were added and the layers separated through a phase separating cartridge. The organic layer was washed with 1 N NaOH, brine, then dried (MgSO₄) and concentrated under vacuum to afford tert-butyl (3-((1-oxo-6-(quinolin-8-ylsulfonyl)phthalazin-2(1H)-yl)methyl)phenyl)carbamate (87 mg) as a pale yellow solid. MS (ES⁺): 543 (M+H)⁺.

### Step 3

TFA (0.17 g, 0.12 mL, 10 Eq, 1.5 mmol) was added to a solution of tert-butyl (3-((1-oxo-6-(quinolin-8-ylsulfonyl)phthalazin-2(1H)-yl)methyl)phenyl)carbamate (82 mg, 1 Eq, 0.15 mmol) in DCM (1 mL) and the mixture was stirred for 1 h. Few drops of 2 N NaOH, followed by sat. NaHCO₃ and DCM were added and the layers separated through a phase separating cartridge. The organic layer was washed with brine, dried (MgSO₄) and concentrated under vacuum to afford the title compound (32.8 mg) as a pale orange solid. ¹H NMR (DMSO-d6) δ: 8.90 (dd, J=4.3, 1.7 Hz, 1H), 8.79 (d, J=1.7 Hz, 1H), 8.73 (dd, J=7.4, 1.4 Hz, 1H), 8.65 (s, 1H), 8.51 (dd, J=8.4, 1.8 Hz, 1H), 8.48-8.36 (m, 3H), 7.93 (dd, J=7.9 Hz, 1H), 7.62 (dd, J=8.4, 4.3 Hz, 1H), 6.98-6.89 (m, 1H), 6.48-6.40 (m, 3H), 5.15 (s, 4H). MS (ES⁺): 443 (M+H)⁺.

### Example 115—2-((6-methoxypyridin-3-yl)methyl)-6-(5-methylpyridin-2-ylsulfonyl)phthalazin-1(2H)-one

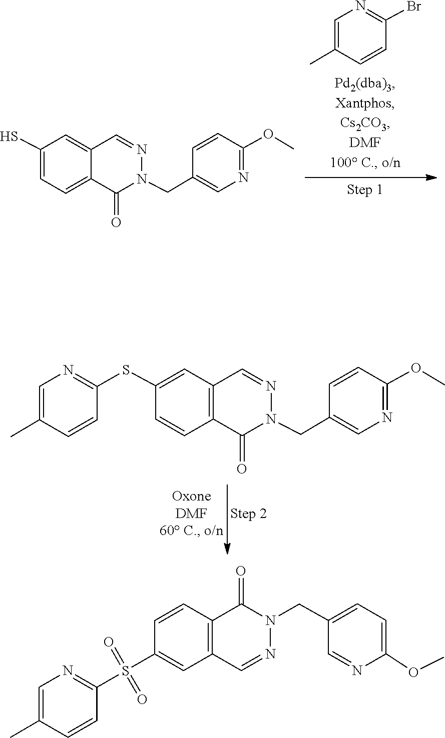

### Step 1

To a mixture of 6-mercapto-2-((6-methoxypyridin-3-yl)methyl)phthalazin-1(2H)-one (Intermediate 40, 500 mg, 1.7 mmol), Pd₂(dba)₃ (156 mg, 0.17 mmol), Xantphos (197 mg, 0.34 mmol), Cs₂CO₃ (1.66 g, 5.1 mmol) in DMF (20 mL) was added 2-bromo-5-methylpyridine (434 mg, 2.55 mmol) at RT, and the reaction mixture was stirred at 100° C. overnight under N₂. After LCMS indicated the reaction to be complete, the reaction mixture was filtered through Celite and washed with EtOAc. The filtrate was diluted with water (70 mL) and extracted with EtOAc (10 mL×3). The combined organic layer was washed with brine, dried over Na₂SO₄, filtered and concentrated at 45° C. under reduced pressure. The residue was purified by flash column chromatography to give 2-((6-methoxypyridin-3-yl)methyl)-6-(5-methylpyridin-2-ylthio)phthalazin-1(2H)-one (270 mg) as a yellow solid. MS (ES⁺): 391.2 (M+H)⁺

### Step 2

To a solution of 2-((6-methoxypyridin-3-yl)methyl)-6-(5-methylpyridin-2-ylthio)phthalazin-1(2H)-one (270 mg, 0.7 mmol) in DMF (3 mL) was added OXONE® (1.29 g, 2.1 mmol) at RT, and the reaction mixture was stirred at 60° C. overnight. After LCMS indicated the reaction completed, the reaction mixture was filtered and washed by DCM (10 mL). The filtrate was diluted with water (20 mL) and extracted with DCM (10 mL×3). The organic layer was concentrated at 30° C. under reduced pressure and the residue was purified by prep-HPLC. The fractions were concentrated at 42° C. under reduced pressure to remove MeCN, and the residue was lyophilized to afford the title compound (52.20 mg) as white solid. ¹H NMR (400 MHz, DMSO-d6) δ: 8.63 (s, 2H), 8.51 (d, J=1.6 Hz, 1H), 8.42 (d, J=8.4 Hz, 1H), 8.26 (dd, J=8.8, 2.0 Hz, 1H), 8.17 (s, 1H), 8.16 (d, J=4.8 Hz, 2H), 7.96 (dd, J=8.0, 1.2 Hz, 1H), 7.65 (dd, J=8.4, 2.4 Hz, 1H), 6.73 (d, J=8.4 Hz, 1H), 5.24 (s, 2H), 3.78 (s, 3H), 2.34 (s, 3H). MS (ES⁺): 423.0 (M+H)⁺

### Example 116—6-(3-methoxyphenylsulfonyl)-2-((6-methoxypyridin-3-yl)methyl)phthalazin-1(2H)-one

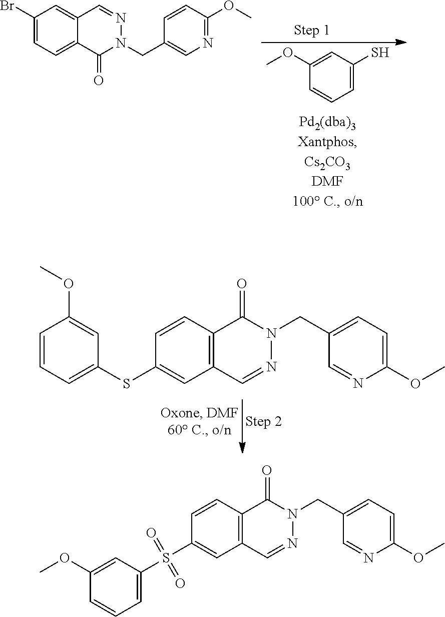

### Step 1

To a mixture of 6-bromo-2-((6-methoxypyridin-3-yl)methyl)phthalazin-1(2H)-one (Intermediate 39, 345 mg, 1.0 mmol), Pd₂(dba)₃ (91.5 mg, 0.1 mmol), Xantphos (115.6 mg, 0.2 mmol), Cs₂CO₃ (975 mg, 3.0 mmol) in DMF (20 mL) was added 3-methoxybenzenethiol (420 mg, 3.0 mmol) at RT, and the reaction mixture was stirred at 100° C. overnight under N₂. After LCMS indicated the reaction was complete, the reaction mixture was filtered through Celite and washed with EtOAc.

The filtrate was diluted with water (70 mL) and extracted with EtOAc (10 mL×3). The combined organic layer was washed by brine, dried over Na₂SO₄, filtered and concentrated at 45° C. under reduced pressure. The residue was purified by flash column chromatography to give 6-(3-methoxyphenylthio)-2-((6-methoxypyridin-3-yl)methyl)phthalazin-1(2H)-one (270 mg) as a yellow solid. MS (ES⁺): 406.0 (M+H)⁺

### Step 2

To a solution of 6-(3-methoxyphenylthio)-2-((6-methoxypyridin-3-yl)methyl)phthalazin-1(2H)-one (270 mg, 0.7 mmol) in DMF (3 mL) was added OXONE® (1.29 g, 2.1 mmol) at RT, and the reaction mixture was stirred at 60° C. overnight. After LCMS indicated the reaction was complete, the reaction mixture was filtered and washed by DCM (10 mL). The filtrate was diluted with water (20 mL) and extracted with DCM (10 mL×3). The organic layer was concentrated at 30° C. under reduced pressure and the residue was purified by prep-HPLC. The fractions were concentrated at 42° C. under reduced pressure to remove MeCN, and the residue was lyophilized to give the title compound (104.8 mg) as white solid. ¹H NMR (400 MHz, DMSO-d6) δ: 8.69 (s, 1H), 8.61 (s, 1H), 8.42 (d, J=8.4 Hz, 1H), 8.33 (d, J=8.4 Hz, 1H), 7.67 (dd, J=2.4 Hz, 8.4 Hz 1H), 7.57 (d, J=5.2 Hz, 2H), 7.48 (s, 1H), 7.31-7.27 (m, 1H), 6.75 (d, J=8.4 Hz, 1H), 5.26 (s, 2H), 3.83 (s, 3H), 3.79 (s, 3H). MS (ES⁺): 438.0 (M+H)⁺

### Example 117—3-(2-((6-methoxypyridin-3-yl)methyl)-1-oxo-1,2-dihydrophthalazin-6-ylsulfonyl)thiophene-2-carboxamide

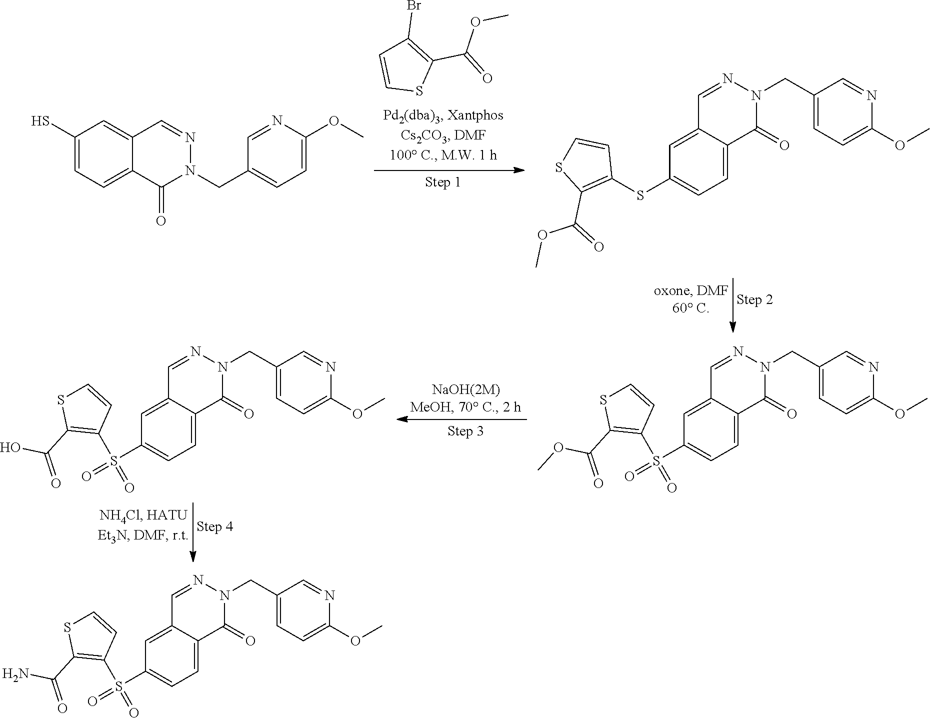

### Step 1

To the solution of 6-mercapto-2-((6-methoxypyridin-3-yl)methyl)phthalazin-1(2H)-one (Intermediate 40, 400 mg, 1.34 mmol), methyl 3-bromothiophene-2-carboxylate (246 mg, 1.12 mmol), Pd₂(dba)₃ (92 mg, 0.1 mmol), Xantphos (116 mg, 0.2 mmol), Cs₂CO₃ (1 g, 3.36 mmol) in DMF (3 mL) at RT, and the reaction mixture was stirred at 100° C. for 1 h under microwave irritation. After LCMS indicated the reaction completed, the reaction mixture was filtered. The mixture was added EtOAc (5 mL), the combined organic layer was washed by brine, separated and extracted with ethyl acetate (5 mL×3). Dried over Na₂SO₄, filtered and concentrated at 45° C. under reduced pressure, the residue was purified by flash column chromatography to give methyl 3-(2-((6-methoxypyridin-3-yl)methyl)-1-oxo-1,2-dihydrophthalazin-6-ylthio)thiophene-2-carboxylate (150 mg) as a yellow solid. MS (ES⁺): 439.9 (M+H)⁺

### Step 2

To a solution of methyl 3-(2-((6-methoxypyridin-3-yl)methyl)-1-oxo-1,2-dihydrophthalazin-6-ylthio) thiophene-2-carboxylate (150 mg, 0.34 mmol) in DMF (3 mL) was added OXONE® (5.2 g, 8.5 mmol) at RT, and the reaction mixture was stirred at 60° C. for 4 days. After LCMS indicated the reaction completed, the reaction mixture was filtered and extracted with DCM (10 mL×3), the organic layer was concentrated at 30° C. under reduced pressure. The residue was purified by flash column chromatography to give methyl 3-(2-((6-methoxypyridin-3-yl)methyl)-1-oxo-1,2-dihydrophthalazin-6-ylsulfonyl)thiophene-2-carboxylate (120 mg) as white solid. MS (ES⁺): 471.8 (M+H)⁺

### Step 3

A mixture of methyl 3-(2-((6-methoxypyridin-3-yl)methyl)-1-oxo-1,2-dihydrophthalazin-6-ylsulfonyl)thiophene-2-carboxylate (120 mg, 0.26 mmol) and NaOH (0.65 mL, 1.3 mmol, 2M) and MeOH (5 mL) was stirred at 70° C. for 2 h. After LCMS indicated the reaction completed, the mixture was quenched with 2 N HCl. The mixture was concentrated at 40° C. to remove organic solvent; the residue was separated and extracted with ethyl acetate (10 mL×3). The separated organics were washed with brine, dried over Na₂SO₄ and filtered. The filtrate was concentrated 40° C. under reduced pressure to give 3-(2-((6-methoxypyridin-3-yl)methyl)-1-oxo-1,2-dihydrophthalazin-6-ylsulfonyl)thiophene-2-carboxylic acid (100 mg, 86%) as yellow oil. MS (ES⁺): 457.8 (M+H)⁺

### Step 4

A mixture of 3-(2-((6-methoxypyridin-3-yl)methyl)-1-oxo-1,2-dihydrophthalazin-6-ylsulfonyl)thiophene-2-carboxylic acid (100 mg 0.22 mmol), NH₄Cl (47 mg, 0.88 mmol), HATU (167 mg, 0.44 mmol), and Et₃N (89 mg, 0.88 mmol) in DMF (5 mL) was stirred at RT for 16 h. After LCMS indicated the reaction completed, the residue was purified by prep-HPLC. The fractions were concentrated at 42° C. under reduced pressure to remove MeCN, and the residue was lyophilized to give the title compound (24.33 mg) as white solid. ¹H NMR (400 MHz, DMSO-d6) δ: 8.64 (d, J=1.2 Hz, 1H), 8.62 (s, 1H), 8.43 (d, J=8.4 Hz, 1H), 8.37 (dd, J=8.4, 2.0 Hz, 1H), 8.20 (d, J=2.0 Hz, 2H), 7.87 (br, 1H), 7.83 (d, J=5.2 Hz, 1H), 7.69 (d, J=8.8, 2.4 Hz, 1H), 7.56 (d, J=5.2 Hz, 1H), 6.77 (d, J=8.8 Hz, 1H), 5.28 (s, 2H), 3.81 (s, 3H). MS (ES⁺): 456.8 (M+H)⁺

### Example 118—4-(2-((6-methoxypyridin-3-yl)methyl)-1-oxo-1,2-dihydrophthalazin-6-ylsulfonyl)thiophene-2-carboxamide

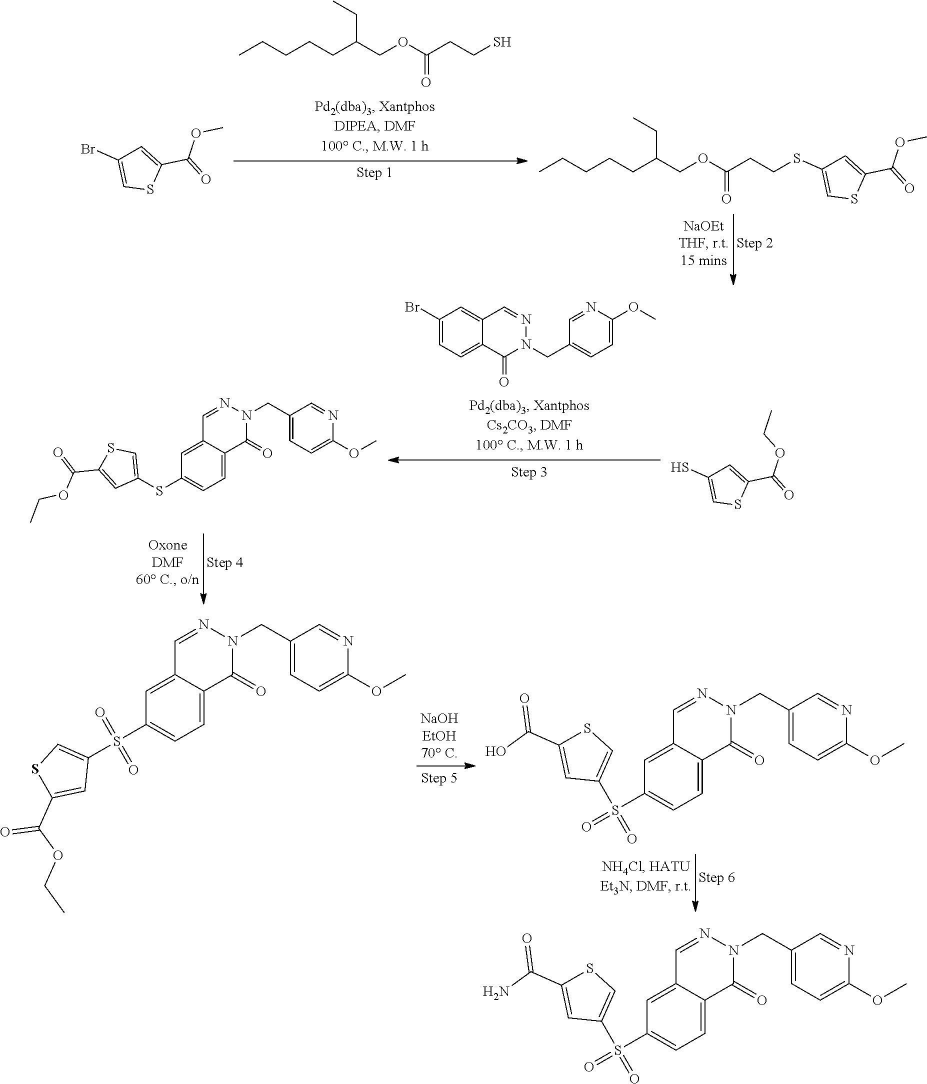

### Step 1

To a solution of methyl 4-bromothiophene-2-carboxylate (450 mg, 2 mmol), Pd₂(dba)₃ (183 mg, 0.2 mmol), Xantphos (231.2 mg, 0.4 mmol), DIPEA (774 mg, 6 mmol) in DMF (5 mL) was added 2-ethylhexyl 3-mercaptopropanoate (510 mg, 2.2 mmol) at RT under N₂, and the reaction mixture was stirred at 100° C. for 1 h. After LCMS indicated the reaction to be complete, the reaction mixture was filtered through Celite and washed with EtOAc. The filtrate was diluted with water (10 mL) and extracted with EtOAc (10 mL×3). The combined organic layer was washed with brine, dried over Na₂SO₄, filtered and concentrated at 45° C. under reduced pressure. The residue was purified by flash column chromatography to give methyl 4-(3-(2-ethylheptyloxy)-3-oxopropylthio)thiophene-2-carboxylate (600 mg, 79%) as a yellow solid. ¹H NMR (400 MHz, DMSO-d6) δ: 7.71 (dd, J=12.8, 1.6 Hz, 1H), 7.45-7.41 (m, 1H), 4.03-4.00 (m, 2H), 3.89 (d, J=2.4 Hz, 3H), 3.09 (t, J=7.2 Hz, 2H), 2.61 (t, J=7.6 Hz, 2H), 1.60-1.55 (m, 1H), 1.37 (t, J=7.2 Hz, 2H), 1.33-1.26 (m, 8H), 0.91-0.87 (m, 6H).

### Step 2

To a solution of methyl 4-(3-(2-ethylheptyloxy)-3-oxopropylthio)thiophene-2-carboxylate (600 mg, 1.6 mmol) in THF (10 mL) was added NaOEt (0.8 mL, 2.5 N in EtOH, 2 mmol) at RT, and the reaction mixture was stirred at room temperature for 15 min. After LCMS indicated the reaction to be complete, the reaction mixture was quenched and pH adjusted to 5 by drop-wise addition of aq. 0.5 N HCl and extracted with EtOAc (20 mL×2). The combined organic layer was washed with brine, dried over Na₂SO₄, filtered and concentrated at 40° C. under reduced pressure to give ethyl 4-mercaptothiophene-2-carboxylate (250 mg) as brown solid. ¹H NMR (400 MHz, CDCl₃) δ: 7.72 (d, J=1.6 Hz, 1H), 7.42 (dd, J=2.8, 1.2 Hz, 1H), 4.35 (dd, J=14.4, 7.2 Hz, 2H), 1.26 (t, J=7.2 Hz, 3H).

### Step 3

To a mixture of ethyl 4-mercaptothiophene-2-carboxylate (250 mg, 1.5 mmol), Pd₂(dba)₃ (137 mg, 0.15 mmol), Xantphos (173 mg, 0.3 mmol), Cs₂CO₃ (978 mg, 3 mmol) in DMF (5 mL) was added 6-bromo-2-((6-methoxypyridin-3-yl)methyl)phthalazin-1(2H)-one (Intermediate 39, 518 mg, 1.5 mmol) at RT, and the reaction mixture was stirred at 100° C. for 1 hour under N₂. After LCMS indicated the reaction is completed, the reaction mixture was filtered through Celite and washed with EtOAc. The filtrate was diluted with water (10 mL) and extracted with EtOAc (10 mL×3).

The combined organic layer was washed by brine, dried over Na₂SO₄, filtered and concentrated at 45° C. under reduced pressure. The residue was purified by flash column chromatography to give ethyl 4-(2-((6-methoxypyridin-3-yl)methyl)-1-oxo-1,2-dihydrophthalazin-6-ylthio)thiophene-2-carboxylate (207 mg) as a yellow solid. LCMS: (System 2, Method D) Rt=0.822 min; MS Calcd.: 453.1; MS Found: 454.0 [M+H]⁺.

### Step 4

To a solution of ethyl 4-(2-((6-methoxypyridin-3-yl)methyl)-1-oxo-1,2-dihydrophthalazin-6-ylthio) thiophene-2-carboxylate (207 mg, 0.46 mmol) in DMF (4 mL) was added OXONE® (858 mg, 1.4 mmol) at RT, and the reaction mixture was stirred at 60° C. overnight. After LCMS indicated the reaction to be complete, the reaction mixture was filtered through Celite and washed with EtOAc. The filtrate was diluted with water (10 mL) and extracted with EtOAc (10 mL×3). The combined organic extracts were washed with brine, dried over Na₂SO₄, filtered and concentrated at 45° C. under reduced pressure. The residue was purified by flash column chromatography to give ethyl 4-(2-((6-methoxypyridin-3-yl)methyl)-1-oxo-1,2-dihydrophthalazin-6-ylsulfonyl)thiophene-2-carboxylate (200 mg) as white solid. MS (ES⁺): 485.8 (M+H)⁺

### Step 5

To the solution of ethyl 4-(2-((6-methoxypyridin-3-yl)methyl)-1-oxo-1,2-dihydrophthalazin-6-yl sulfonyl)thiophene-2-carboxylate (200 mg, 0.41 mmol) in EtOH (3 mL) was added NaOH (48 mg, 1.2 mmol) at RT, and the reaction mixture was stirred at 70° C. for 5 h. After LCMS indicated the reaction to be complete, the mixture was concentrated at 30° C. under reduced pressure to remove MeOH. The residue was quenched with water (10 mL), adjusted to pH=4 with 0.5 N HCl aqueous solution, and extracted with DCM (10 mL×2). The organic layer was washed with brine, dried over Na₂SO₄, filtered and concentrated at 30° C. under reduced pressure to give 4-(2-((6-methoxypyridin-3-yl)methyl)-1-oxo-1,2-dihydrophthalazin-6-ylsulfonyl)thiophene-2-carboxylic acid (140 mg) as a white solid, which was used to the next step directly. MS (ES⁺): 457.8 (M+H)⁺

### Step 6

To a solution of 5-methoxy-2-((6-(4-methoxyphenylsulfonyl)-1-oxophthalazin-2(1H)-yl)methyl)benzoic acid (200 mg, 0.4 mmol), NH₄Cl (118 mg, 2 mmol) and HATU (208 mg, 0.8 mmol) in DMF (5 mL) was added Et₃N (202 mg, 2 mmol) at RT, and the reaction mixture was stirred at RT for 3 hours. After LCMS indicated the reaction completed, the reaction mixture was extracted with DCM (10 mL×3). The organic layer was concentrated at 40° C. under reduced pressure and the residue was purified by prep-HPLC. The relevant fractions were concentrated at 42° C. under reduced pressure to remove MeCN, and the residue was lyophilized to give the title compound (77.10 mg) as white solid. ¹H NMR (400 MHz, DMSO-d6) δ: 8.73 (d, J=1.2 Hz, 1H), 8.65 (s, 1H), 8.63 (d, J=1.6 Hz, 1H), 8.48 (d, J=8.4 Hz, 1H), 8.29 (dd, J=8.4, 1.6 Hz, 1H), 8.20 (d, J=20 Hz, 1H), 8.18 (br, 1H), 8.12 (d, J=1.6 Hz, 1H), 8.69 (dd, J=8.4, 2.4 Hz, 2H), 6.77 (d, J=8.4 Hz, 1H), 5.28 (s, 2H), 3.81 (s, 3H). MS (ES⁺): 456.9 (M+H)⁺

### Example 119—2-(3-aminobenzyl)-6-(phenylsulfinyl)phthalazin-1(2H)-one

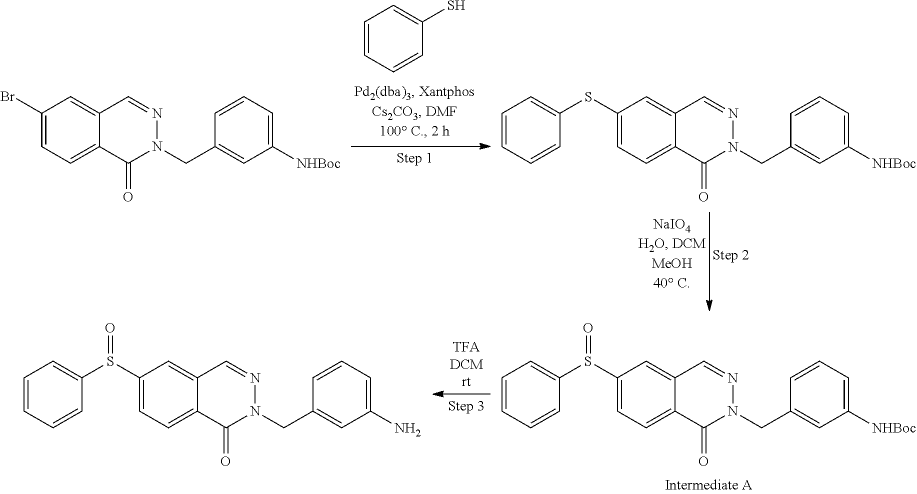 [SMILES: Sc1ccccc1 ; CC(C)(C)OC(=O)Nc1cccc(Cn2ncc3cc(Br)ccc3c2=O)c1 ; CC(C)(C)OC(=O)Nc1cccc(Cn2ncc3cc(Sc4ccccc4)ccc3c2=O)c1 ; Nc1cccc(Cn2ncc3cc(S(=O)c4ccccc4)ccc3c2=O)c1 ; CC(C)(C)OC(=O)Nc1cccc(Cn2ncc3cc(S(=O)c4ccccc4)ccc3c2=O)c1]

### Step 1

A suspension of tert-butyl (3-((6-bromo-1-oxophthalazin-2(1H)-yl)methyl)phenyl)carbamate (Intermediate 41, 635 mg, 99% Wt, 1 Eq, 1.46 mmol) and cesium carbonate (1.43 g, 3.00 Eq, 4.38 mmol) in DMF (8 mL) was sparged with N₂ for 15 minutes. Benzenethiol (483 mg, 450 μL, 3.00 Eq, 4.38 mmol), Xantphos (84.5 mg, 0.1 Eq, 146 μmol) and Pd₂dba₃ (66.9 mg, 0.05 Eq, 73.0 μmol) were added sequentially and the reaction mixture was stirred at 100° C. under N₂ for 2 h, then allowed to cool to RT. The reaction mixture was partitioned between EtOAc (100 mL) and 50% v/v aq NaHCO₃ (100 mL). The organic layer was collected and the aqueous was extracted with EtOAc (2×100 mL). The combined organic extracts were washed with brine (3×50 mL), dried (MgSO₄) and concentrated in vacuo to afford the crude product. The crude product was purified by chromatography on silica gel to afford tert-butyl (3-((1-oxo-6-(phenylthio)phthalazin-2(1H)-yl)methyl)phenyl)carbamate (626 mg) as a thick orange oil. MS (ES⁺): 460 (M+H)⁺

### Step 2

A solution of tert-butyl (3-((1-oxo-6-(phenylthio)phthalazin-2(1H)-yl)methyl)phenyl)carbamate (1.093 g, 52% Wt, 1 Eq, 1.237 mmol) in DCM (3 mL) was treated with a suspension of sodium periodate (805 mg, 3.04 Eq, 3.76 mmol) in water (5 mL). The biphasic mixture was homogenised with MeOH (25 mL) and then stirred at 40° C. for 18 h. The reaction mixture was diluted with DCM (30 mL) and washed with water (30 mL). The organic layer was collected and the aqueous was extracted with DCM (2×30 mL), The combined organic extracts were dried (phase separator) and concentrated in vacuo to afford the crude product. The crude product was purified by chromatography on silica gel to afford tert-butyl (3-((1-oxo-6-(phenylsulfinyl)phthalazin-2(1H)-yl)methyl)phenyl)carbamate (369 mg) as a pale pink solid. MS (ES⁺): 422 (M+H)⁺

### Step 3

A solution of tert-butyl (3-((1-oxo-6-(phenylsulfinyl)phthalazin-2(1H)-yl)methyl)phenyl)carbamate (Intermediate A, 50 mg, 93% Wt, 1 Eq, 98 μmol) in DCM (1 mL) was treated with 2,2,2-trifluoroacetic acid (0.22 g, 0.15 mL, 20 Eq, 2.0 mmol). The reaction mixture was stirred at RT for 1 h, diluted with DCM (4 mL), quenched with 2 M NaOH(aq) (5 mL) and then stirred for 1 h. The organic layer was collected and the aqueous was extracted with DCM (5 mL). The combined organic extracts were dried (phase separator) and concentrated in vacuo to afford the crude product. The crude product was purified by chromatography on silica gel to afford the title compound (37.3 mg) as a flocculent white solid. ¹H NMR (400 MHz, DMSO-d6) δ 8.58 (s, 1H), 8.42 (d, 1H), 8.36 (d, 1H), 8.08 (dd, 1H), 7.83-7.77 (m, 2H), 7.59-7.51 (m, 3H), 6.92 (t, 1H), 6.45-6.39 (m, 3H), 5.16 (s, 2H), 5.02 (s, 2H). MS (ES⁺): 422 (M+H)⁺

### Example 120—2-(3-aminobenzyl)-6-(phenylsulfonimidoyl)phthalazin-1(2H)-one

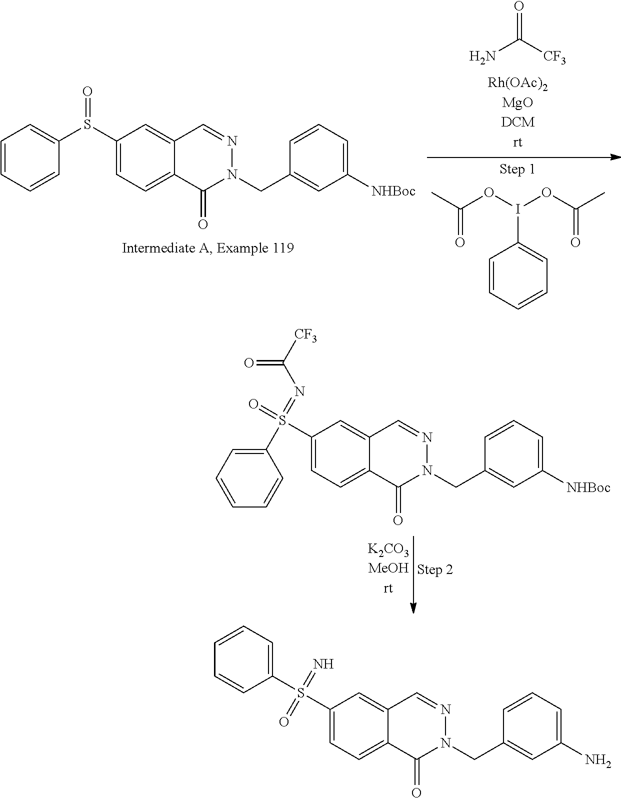 [SMILES: NC(=O)C(F)(F)F ; CC(C)(C)OC(=O)Nc1cccc(Cn2ncc3cc(S(=O)c4ccccc4)ccc3c2=O)c1 ; CC(=O)OI(OC(C)=O)c1ccccc1 ; CC(C)(C)OC(=O)Nc1cccc(Cn2ncc3cc(S(=O)(=NC(=O)C(F)(F)F)c4ccccc4)ccc3c2=O)c1 ; N=S(=O)(c1ccccc1)c1ccc2c(=O)n(Cc3cccc(N)c3)ncc2c1]

### Step 1

(Diacetoxyiodo)benzene (102 mg, 1.5 Eq, 315 μmol) and diacetoxyrhodium (2.32 mg, 0.025 Eq, 5.26 μmol) were added to a suspension of tert-butyl (3-((1-oxo-6-(phenylsulfinyl)phthalazin-2(1H)-yl)methyl)phenyl)carbamate (Intermediate A, Example 119, 100 mg, 1 Eq, 210 μmol), 2,2,2-trifluoroacetamide (47.5 mg, 2 Eq, 421 μmol) and magnesium oxide (33.9 mg, 4 Eq, 841 μmol) in DCM (1 mL) and the mixture was stirred overnight. DCM and water were added and the resulting emulsion was filtered. The filtrate was washed with brine, dried (MgSO₄), then absorbed on silica. The crude product was purified by chromatography on silica gel to afford tert-butyl (3-((1-oxo-6-(N-(2,2,2-trifluoroacetyl)phenylsulfonimidoyl)phthalazin-2(1H)-yl)methyl)phenyl)carbamate (53 mg, 86 μmol, 41%) as an off-white solid. MS (ES⁺): 531 (M−tBu+H)⁺

### Step 2

A mixture of tert-butyl (3-((1-oxo-6-(N-(2,2,2-trifluoroacetyl)phenylsulfonimidoyl)phthalazin-2(1H)-yl)methyl)phenyl)carbamate (53 mg, 1 Eq, 90 μmol) and potassium carbonate (0.12 g, 10 Eq, 0.90 mmol) in MeOH (0.5 mL) was stirred for 45 minutes. Water and DCM were added and the layers separated through a phase separating cartridge. The organic layer was washed with brine, dried (MgSO₄) and concentrated under vacuum to afford crude tert-butyl (3-((1-oxo-6-(phenylsulfonimidoyl)phthalazin-2(1H)-yl)methyl)phenyl)carbamate. The product was dissolved in DCM (0.5 mL) and TFA (0.10 g, 70 μL, 10 Eq, 0.90 mmol) was added. The mixture was stirred for 30 minutes, and 2 N NaOH, Sat. NaHCO₃ and DCM were added. The layers were separated through a phase separating cartridge. The organic layer was washed with brine, dried (MgSO₄) and concentrated under vacuum to afford the title compound (18.7 mg) as a pale grey solid. ¹H NMR (DMSO-d6) δ: 8.64 (d, J=1.8 Hz, 1H), 8.63 (s, 1H), 8.39 (d, J=8.4 Hz, 1H), 8.31 (dd, J=8.4, 1.8 Hz, 1H), 8.06-7.97 (m, 2H), 7.68-7.54 (m, 3H), 6.96-6.88 (m, 1H), 6.45-6.38 (m, 3H), 5.40 (s, 1H), 5.16 (s, 2H), 5.01 (s, 2H). MS (ES⁺): 391 (M+H)⁺

### Example 121—2-(3-aminobenzyl)-6-(pyridin-3-ylsulfonyl)phthalazin-1(2H)-one

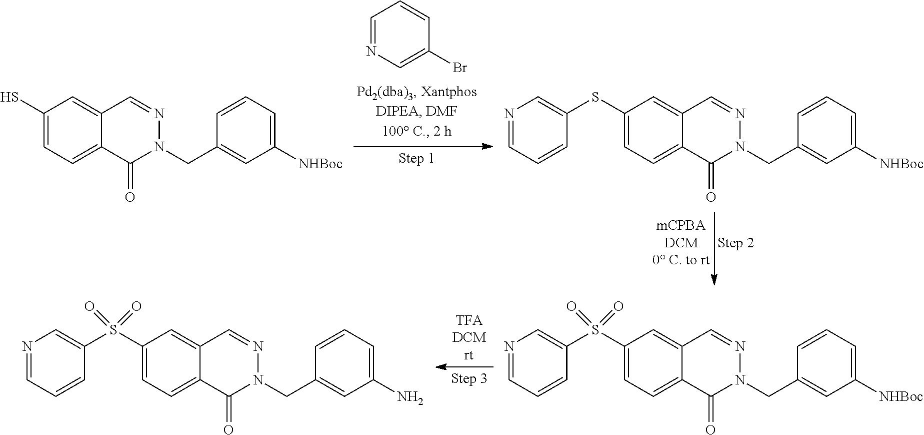 [SMILES: Brc1cccnc1 ; CC(C)(C)OC(=O)Nc1cccc(Cn2ncc3cc(S)ccc3c2=O)c1 ; CC(C)(C)OC(=O)Nc1cccc(Cn2ncc3cc(Sc4cccnc4)ccc3c2=O)c1 ; Nc1cccc(Cn2ncc3cc(S(=O)(=O)c4cccnc4)ccc3c2=O)c1 ; CC(C)(C)OC(=O)Nc1cccc(Cn2ncc3cc(S(=O)(=O)c4cccnc4)ccc3c2=O)c1]

### Step 1

A mixture of tert-butyl (3-((6-mercapto-1-oxophthalazin-2(1H)-yl)methyl)phenyl)carbamate (Intermediate 42, 0.110 g, 1 Eq, 287 μmol), 3-bromopyridine (49.9 mg, 30.4 μL, 1.1 Eq, 316 μmol), DIPEA (51.9 mg, 70.0 μL, 1.4 Eq, 402 μmol), Pd₂dba₃ (26.3 mg, 0.1 Eq, 28.7 μmol) and Xantphos (33.2 mg, 0.2 Eq, 57.4 μmol) in DMF (2.5 mL) was heated to 110° C. for 18 h under N₂, then cooled to RT. The mixture was diluted with EtOAc and the resulting solution filtered through a celite pad and washed with EtOAc. The filtrate was washed with brine (10 mL). The organic phase was collected, dried (MgSO₄) and evaporated under reduced pressure. The crude product was purified by chromatography on silica gel to afford tert-butyl (3-((1-oxo-6-(pyridin-3-ylthio)phthalazin-2(1H)-yl)methyl)phenyl)carbamate (0.105 g) as a white solid. MS (ES⁺): 483 (M+Na)⁺

### Step 2

A stirred solution of tert-butyl (3-((1-oxo-6-(pyridin-3-ylthio)phthalazin-2(1H)-yl)methyl)phenyl)carbamate (0.105 g, 1 Eq, 228 μmol) in dry DCM (4 mL) was cooled to 0° C. m-CPBA (102 mg, 77% Wt, 2.0 Eq, 456 μmol) was added and the reaction mixture was stirred for a further 2 h while warming up to RT. The reaction mixture was diluted with DCM (2 mL) and washed with sat. aq. NaHCO₃ (3 mL). The organic was collected and the aqueous was extracted with EtOAc (2×5 mL). The combined organic extracts were dried (phase separator) and concentrated in vacuo to afford crude tert-butyl (3-((1-oxo-6-(pyridin-3-ylsulfonyl)phthalazin-2(1H)-yl)methyl)phenyl)carbamate (0.160 g) as a yellow solid. MS (ES⁺): 515 (M+Na)⁺

### Step 3

TFA (126 mg, 85.1 μL, 10 Eq, 1.10 mmol) was added dropwise to a stirred solution of tert-butyl (3-((1-oxo-6-(pyridin-3-ylsulfonyl)phthalazin-2(1H)-yl)methyl)phenyl)carbamate (0.160 g, 34% Wt, 1 Eq, 110 μmol) in DCM (2 mL) at 0° C. The resulting mixture stirred for 1 h while warming to RT before being treated with a saturated solution of NaHCO₃ (4 mL). The organic phase was collected, dried (phase separator) and then evaporated under reduced pressure. The crude product was purified by chromatography on silica gel to afford the title compound (0.009 g) as an off-white solid. ¹H NMR (400 MHz, DMSO-d6) δ 9.22 (d, J=2.5 Hz, 1H), 8.90 (dd, J=4.8, 1.6 Hz, 1H), 8.77 (d, J=1.8 Hz, 1H), 8.62 (s, 1H), 8.49-8.35 (m, 4H), 7.76-7.65 (m, 1H), 6.96 (t, J=7.8 Hz, 1H), 6.53-6.44 (m, 2H), 5.18 (s, 2H) (Exchangeable —NHs not observed). MS (ES⁺): 393 (M+H)⁺

### Example 122—2-((6-methoxypyridin-3-yl)methyl)-1-oxo-N-phenyl-1,2-dihydrophthalazine-6-sulfonamide

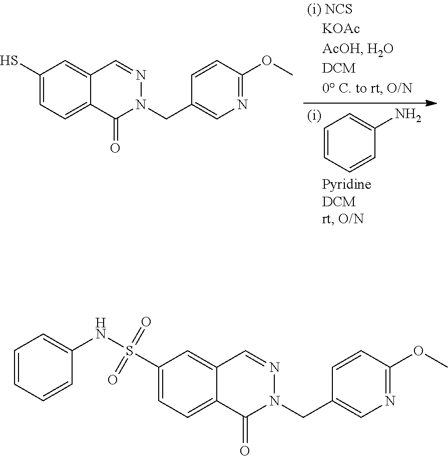 [SMILES: COc1ccc(Cn2ncc3cc(S)ccc3c2=O)cn1 ; Nc1ccccc1 ; COc1ccc(Cn2ncc3cc(S(=O)(=O)Nc4ccccc4)ccc3c2=O)cn1]

### Step (i)

NCS (268 mg, 5 Eq, 2.00 mmol) and potassium acetate (19.7 mg, 0.5 Eq, 200 μmol) were added to a stirred solution of 6-mercapto-2-((6-methoxypyridin-3-yl)methyl)phthalazin-1(2H)-one (Intermediate 40, 0.120 g, 1 Eq, 401 μmol) in a mixture of acetic acid (0.8 mL), water (0.5 mL), and DCM (0.9 mL) at 0° C. The resulting mixture was stirred overnight while warming up to RT. The mixture was then diluted with DCM (2 mL) and washed with water (3×2 mL). The organic layer was dried over MgSO₄ and concentrated to give crude 2-((6-methoxypyridin-3-yl)methyl)-1-oxo-1,2-dihydrophthalazine-6-sulfonyl chloride. The product was used without further purification in the next step. MS (ES)⁺: 348 (M+-Cl +H₂O)⁺

### Step (ii)

Pyridine (47.7 mg, 48.8 μL, 1.5 Eq, 603 μmol) was added to a stirred solution of 2-((6-methoxypyridin-3-yl)methyl)-1-oxo-1,2-dihydrophthalazine-6-sulfonyl chloride (0.147 g, 1 Eq, 402 μmol) in DCM (1.0 mL) at RT. The resulting mixture was stirred for 40 minutes before aniline (37.4 mg, 36.7 μL, 1 Eq, 402 μmol) was added. Stirring was continued for a further 18 h, then 1 M HCl (aq.) (0.5 mL) was added, followed by extraction with DCM (3×2 mL). The organic phase was then dried (phase separator) and evaporated under reduced pressure. The crude product was purified by chromatography on silica gel, then on reverse phase Flash C18 to afford the title compound (0.032 g, 75 μmol) as a white solid. ¹H NMR (400 MHz, DMSO-d6) δ 10.58 (s, 1H), 8.59 (s, 1H), 8.44-8.35 (m, 2H), 8.20 (d, J=2.4 Hz, 1H), 8.10 (dd, J=8.4, 1.8 Hz, 1H), 7.69 (dd, J=8.6, 2.5 Hz, 1H), 7.22 (t, J=7.8 Hz, 2H), 7.12-7.00 (m, 3H), 6.76 (d, J=8.5 Hz, 1H), 5.25 (s, 2H), 3.81 (s, 3H). MS (ES)⁺: 423 (M+H)⁺

### Example 123—6-((1H-indazol-4-yl)sulfonyl)-2-((6-methoxypyridin-3-yl)methyl)phthalazin-1(2H)-one

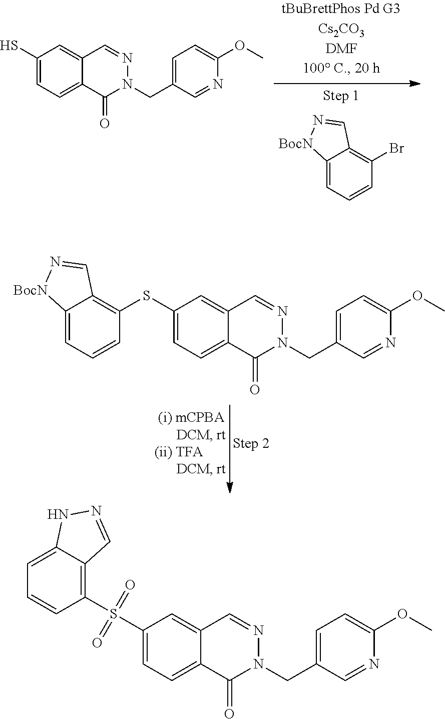 [SMILES: COc1ccc(Cn2ncc3cc(S)ccc3c2=O)cn1 ; CC(C)(C)OC(=O)n1ncc2c(Br)cccc21 ; COc1ccc(Cn2ncc3cc(Sc4cccc5c4cnn5C(=O)OC(C)(C)C)ccc3c2=O)cn1 ; COc1ccc(Cn2ncc3cc(S(=O)(=O)c4cccc5[nH]ncc45)ccc3c2=O)cn1]

### Step 1

tBuBrettPhos Pd G3 (14 mg, 0.1 Eq, 17 μmol) was added to a degassed mixture of 6-mercapto-2-((6-methoxypyridin-3-yl)methyl)phthalazin-1(2H)-one (Intermediate 40, 0.050 g, 1 Eq, 0.17 mmol), tert-butyl 4-bromo-1H-indazole-1-carboxylate (55 mg, 1.1 Eq, 0.18 mmol), and cesium carbonate (0.11 g, 2.0 Eq, 0.33 mmol) in DMF (1.5 mL). The resulting mixture was heated to 100° C. for 20 h. After cooling to RT, the reaction mixture was diluted with water (3 mL) and extracted with DCM (2×10 mL). The organic phase was collected, dried (MgSO₄) and evaporated under reduced pressure. The crude product was purified by chromatography on silica gel to afford tert-butyl 4-((2-((6-methoxypyridin-3-yl)methyl)-1-oxo-1,2-dihydrophthalazin-6-yl)thio)-1H-indazole-1-carboxylate (0.023 g, 41 μmol, 25%) as an orange gum. MS (ES⁺): 416 (M-CO₂tBu)⁺

### Step 2 (i)

m-CPBA (23 mg, 3 Eq, 0.13 mmol) was added to a stirred solution of tert-butyl 4-((2-((6-methoxypyridin-3-yl)methyl)-1-oxo-1,2-dihydrophthalazin-6-yl)thio)-1H-indazole-1-carboxylate (0.023 g, 1 Eq, 45 μmol) in DCM (2 mL) at RT and the mixture was stirred for 1 h. The reaction mixture was diluted with DCM (5 mL) and washed with saturated NaHCO₃ (5 mL) and water (5 mL). The organic phase was collected, dried (phase separator) and evaporated under reduced pressure to afford crude tert-butyl 4-((2-((6-methoxypyridin-3-yl)methyl)-1-oxo-1,2-dihydrophthalazin-6-yl)sulfonyl)-1H-indazole-1-carboxylate.

### Step 2 (ii)

The product was dissolved in DCM (2 mL) and treated with TFA (51 mg, 34 μL, 10 Eq, 0.45 mmol). The resulting mixture was stirred at RT for 2 h, then it was diluted with DCM (5 mL) and washed with a saturated solution of NaHCO₃ (5 mL). The organic phase was collected, dried (phase separator) and evaporated under reduced pressure. The crude product was purified by chromatography on silica gel to afford the title compound (0.015 g) as an off white solid. ¹H NMR (DMSO-d6) δ 13.76 (s, 1H), 8.83 (s, 1H), 8.63 (s, 1H), 8.46 (s, 1H), 8.39-8.36 (m, 2H), 8.17 (d, J=2.5 Hz, 1H), 7.96 (d, J=8.4 Hz, 1H), 7.92 (dd, J=7.4, 0.7 Hz, 1H), 7.69-7.58 (m, 2H), 6.73 (dd, J=8.5, 0.7 Hz, 1H), 5.24 (s, 2H), 3.79 (s, 3H). MS (ES⁺): 448 (M+H)⁺

### Example 124—6-((6-(difluoromethoxy)pyridin-3-yl)sulfonyl)-2-((6-methoxypyridin-3-yl)methyl)phthalazin-1(2H)-one

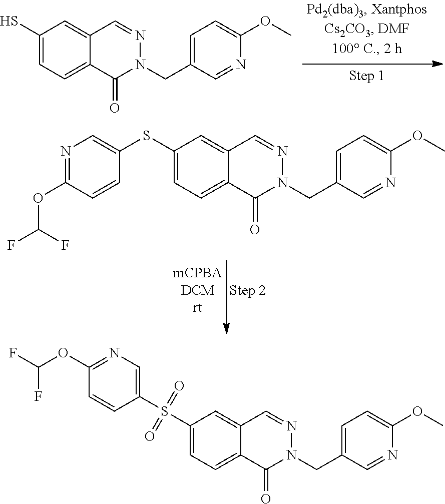

### Step 1

N₂ was bubbled through a mixture of 6-mercapto-2-((6-methoxypyridin-3-yl)methyl)phthalazin-1(2H)-one (Intermediate 40, 50 mg, 1 Eq, 0.17 mmol), 5-bromo-2-(difluoromethoxy)pyridine (45 mg, 1.2 Eq, 0.20 mmol) and cesium carbonate (0.11 g, 2 Eq, 0.33 mmol) in DMF (1 mL) for 5 minutes. Pd₂dba₃ (7.6 mg, 0.05 Eq, 8.4 μmol) and Xantphos (9.7 mg, 0.1 Eq, 17 μmol) were added and the mixture was stirred at 100° C. under N₂ overnight, then allowed to cool to RT. Water and DCM were added and the layers separated through a phase separator. The crude product was purified by chromatography on silica gel to afford 6-((6-(difluoromethoxy)pyridin-3-yl)thio)-2-((6-methoxypyridin-3-yl)methyl)phthalazin-1(2H)-one (7.00 mg) as a white solid. MS (ES⁺): 443 (M+H)⁺

### Step 2

mCPBA (9.75 mg, 70% Wt, 2.5 Eq, 39.6 μmol) was added to a solution of 6-((6-(difluoromethoxy)pyridin-3-yl)thio)-2-((6-methoxypyridin-3-yl)methyl)phthalazin-1(2H)-one (7.00 mg, 1 Eq, 15.8 μmol) in DCM (0.2 mL) and the mixture was stirred for 45 minutes. 2 N NaOH (0.1 mL) was added followed by sat. NaHCO₃ and DCM. The layers were separated through a phase separating cartridge and the organic layer was washed with brine, then absorbed on silica. The crude product was purified by chromatography on silica gel to afford the title compound (3.8 mg) as a white solid. ¹H NMR (DMSO-d6) δ: 8.95 (d, J=2.7 Hz, 1H), 8.73 (d, J=1.8 Hz, 1H), 8.60 (s, 1H), 8.48 (dd, J=8.7, 2.6 Hz, 1H), 8.44 (d, J=8.5 Hz, 1H), 8.38 (dd, J=8.5, 1.9 Hz, 1H), 8.19 (d, J=2.4 Hz, 1H), 7.75 (t, J=71.6 Hz, 1H), 7.67 (dd, J=8.5, 2.5 Hz, 1H), 7.34 (d, J=8.7 Hz, 1H), 6.76 (d, J=8.5 Hz, 1H), 5.27 (s, 2H), 3.80 (s, 3H). MS (ES⁺): 475 (M+H)⁺

### Example 125—3-((6-methoxypyridin-3-yl)methyl)-7-((3-phenyloxetan-3-yl)oxy)pyrido[3,4-d]pyridazin-4(3H)-one

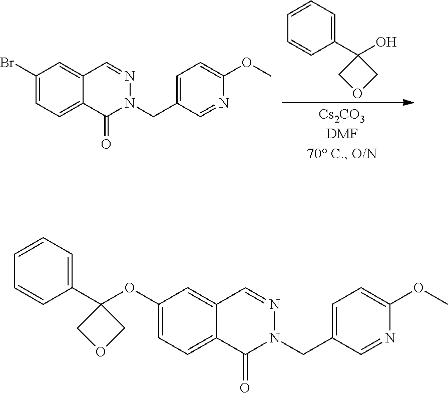

Cesium carbonate (94 mg, 2.0 Eq, 0.29 mmol) was added to a stirred solution of 7-bromo-3-((6-methoxypyridin-3-yl)methyl)pyrido[3,4-d]pyridazin-4(3H)-one (Intermediate 39, 0.050 g, 1 Eq, 0.14 mmol), 3-phenyloxetan-3-ol (32 mg, 1.5 Eq, 0.22 mmol) in DMF (1.0 mL) and the resulting mixture was heated to 70° C. over 18 h. The mixture was allowed to cool to RT and diluted with water (5 mL) then extracted with DCM (2×10 mL). The combined organic layers were dried over MgSO₄ and evaporated to afford a crude product. The crude product was purified by chromatography on silica gel to afford the title compound (0.012 g) as a white solid. ¹H NMR (400 MHz, DMSO-d6) δ 8.87 (s, 1H), 8.39 (s, 1H), 8.16 (d, J=2.4 Hz, 1H), 7.65 (dd, J=8.6, 2.5 Hz, 1H), 7.53 (d, J=7.6 Hz, 2H), 7.39-7.32 (m, 3H), 7.31-7.24 (m, 1H), 6.75 (d, J=8.5 Hz, 1H), 5.19 (s, 2H), 5.09 (d, J=8.0 Hz, 2H), 4.97 (d, J=7.9 Hz, 2H), 3.80 (s, 3H). MS (ES⁺): 417 (M+H)⁺

### Example 126—6-((2,3-dihydrobenzofuran-5-yl)sulfonyl)-2-((6-methoxypyridin-3-yl)methyl)phthalazin-1(2H)-one

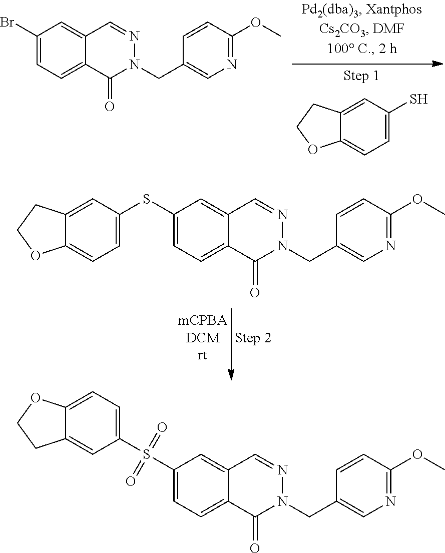 [SMILES: COc1ccc(Cn2ncc3cc(Br)ccc3c2=O)cn1 ; Sc1ccc2c(c1)CCO2 ; COc1ccc(Cn2ncc3cc(Sc4ccc5c(c4)CCO5)ccc3c2=O)cn1 ; COc1ccc(Cn2ncc3cc(S(=O)(=O)c4ccc5c(c4)CCO5)ccc3c2=O)cn1]

### Step 1

Pd₂dba₃ (13.2 mg, 0.05 Eq, 14.4 μmol), Cs₂CO₃ (188 mg, 2 Eq, 578 μmol) and Xantphos (16.7 mg, 0.1 Eq, 28.9 μmol) were added to a stirred solution of 6-bromo-2-((6-methoxypyridin-3-yl)methyl)phthalazin-1(2H)-one (Intermediate 39, 100 mg, 1 Eq, 289 μmol) and 2,3-dihydrobenzofuran-5-thiol (52.8 mg, 1.2 Eq, 347 μmol) in dry DMF (6 mL) under N₂. The reaction was stirred at 90° C. for 2 h, then allowed to cool to RT and diluted with DCM (20 mL). The organic phase was washed with water (20 mL) and brine (20 mL). The organic phase was collected, dried (phase separator) and evaporated under reduced pressure. The crude product was purified by chromatography on silica gel to afford 6-((2,3-dihydrobenzofuran-5-yl)thio)-2-((6-methoxypyridin-3-yl)methyl)phthalazin-1(2H)-one (0.14 g) as a sticky brown oil. MS (ES⁺): 418 (M+H)⁺

Step 2 mCPBA (0.13 g, 3 Eq, 0.76 mmol) was added to a stirred solution of 6-((2,3-dihydrobenzofuran-5-yl)thio)-2-((6-methoxypyridin-3-yl)methyl)phthalazin-1(2H)-one (0.14 g, 76% Wt, 1 Eq, 0.25 mmol) in DCM (6 mL) at RT and the mixture was stirred for 2 h. The reaction mixture was diluted with DCM (10 mL) and washed with saturated NaHCO₃ (10 mL) and water (10 mL). The organic phase was collected, dried (phase separator) and evaporated under reduced pressure. The crude product was purified by prep-HPLC (Basic Method) to afford the title compound (20 mg) as a flocculant white solid. ¹H NMR (400 MHz, DMSO-d6) δ 8.61 (d, J=1.1 Hz, 2H), 8.41 (d, J=8.4 Hz, 1H), 8.26 (dd, J=8.5, 1.9 Hz, 1H), 8.19 (d, J=2.4 Hz, 1H), 7.85 (d, J=2.0 Hz, 1H), 7.78 (dd, J=8.5, 2.2 Hz, 1H), 7.67 (dd, J=8.6, 2.5 Hz, 1H), 6.97 (d, J=8.5 Hz, 1H), 6.76 (d, J=8.5 Hz, 1H), 5.27 (s, 2H), 4.64 (t, J=8.8 Hz, 2H), 3.80 (s, 3H), 3.23 (t, J=8.8 Hz, 2H). MS (ES⁺): 450 (M+H)⁺

### Example 127—6-(2,3-dihydrobenzofuran-5-sulfonimidoyl)-2-((6-methoxypyridin-3-yl)methyl)phthalazin-1(2H)-one

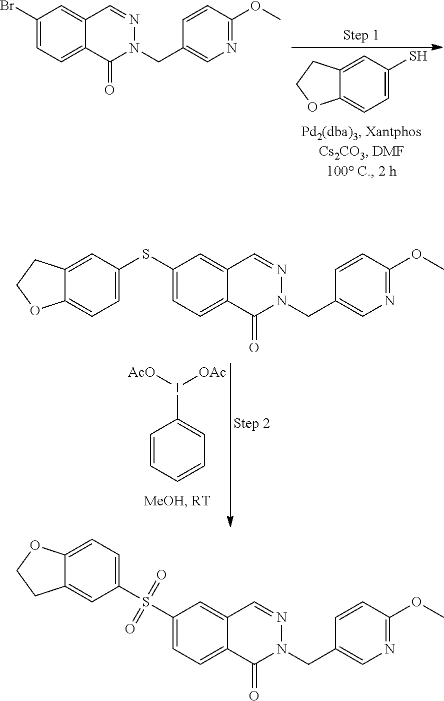 [SMILES: COc1ccc(Cn2ncc3cc(Br)ccc3c2=O)cn1 ; Sc1ccc2c(c1)CCO2 ; COc1ccc(Cn2ncc3cc(Sc4ccc5c(c4)CCO5)ccc3c2=O)cn1 ; CC(=O)OI(OC(C)=O)c1ccccc1 ; COc1ccc(Cn2ncc3cc(S(=O)(=O)c4ccc5c(c4)CCO5)ccc3c2=O)cn1]

### Step 1

N₂ was bubbled through a mixture of 6-bromo-2-((6-methoxypyridin-3-yl)methyl)phthalazin-1(2H)-one (Intermediate 39, 100 mg, 1 Eq, 289 μmol), 2,3-dihydrobenzofuran-5-thiol (44.0 mg, 1 Eq, 289 μmol) and cesium carbonate (188 mg, 2 Eq, 578 μmol) in DMF (1.5 mL) for 5 minutes. Pd₂dba₃ (13.2 mg, 0.05 Eq, 14.4 μmol) and Xantphos (16.7 mg, 0.1 Eq, 28.9 μmol) were added and the mixture stirred at 100° C. under N₂ for 2 h, then allowed to cool to RT and stirred overnight. The mixture was poured into ice/water and DCM was added. The layers were separated through a phase separator and the organic layer was washed with twice with water, twice with brine, then dried (MgSO₄) and concentrated under vacuum to afford crude 6-((2,3-dihydrobenzofuran-5-yl)thio)-2-((6-methoxypyridin-3-yl)methyl)phthalazin-1(2H)-one (117 mg) as an orange solid. MS (ES⁺): 418 (M+H)⁺

### Step 2

Iodobenzene diacetate (0.23 g, 5 Eq, 0.72 mmol) was added to a mixture of 6-((2,3-dihydrobenzofuran-5-yl)thio)-2-((6-methoxypyridin-3-yl)methyl)phthalazin-1(2H)-one (60 mg, 1 Eq, 0.14 mmol) and ammonium carbamate (56 mg, 5 Eq, 0.72 mmol) in DCM (1 mL) and the mixture was stirred overnight. Sat. NaHCO₃ and DCM were added and the layers separated through a phase separator. The organic layer was washed with brine, then absorbed on silica. The crude product was purified by chromatography on silica gel to afford the title compound (22.4 mg, 47 μmol) as a white solid. ¹H NMR (DMSO-d6) δ: 8.61 (s, 1H), 8.56 (d, J=1.8 Hz, 1H), 8.36 (d, J=8.4 Hz, 1H), 8.26 (dd, J=8.4, 1.8 Hz, 1H), 8.18 (d, J=2.4 Hz, 1H), 7.85-7.82 (m, 1H), 7.78 (dd, J=8.5, 2.2 Hz, 1H), 7.67 (dd, J=8.6, 2.5 Hz, 1H), 6.90 (d, J=8.4 Hz, 1H), 6.76 (d, 1H), 5.26 (s, J=8.5, 2H), 5.16 (s, 1H), 4.60 (t, J=8.8, 8.8 Hz, 2H), 3.80 (s, 3H), 3.20 (t, J=8.8, 8.8 Hz, 2H). MS (ES⁺): 449 (M+H)⁺

### Example 128—(R)-6-(2,3-dihydrobenzofuran-5-sulfonimidoyl)-2-((6-methoxypyridin-3-yl)methyl)phthalazin-1(2H)-one; and

### Example 129—(S)-6-(2,3-dihydrobenzofuran-5-sulfonimidoyl)-2-((6-methoxypyridin-3-yl)methyl)phthalazin-1(2H)-one

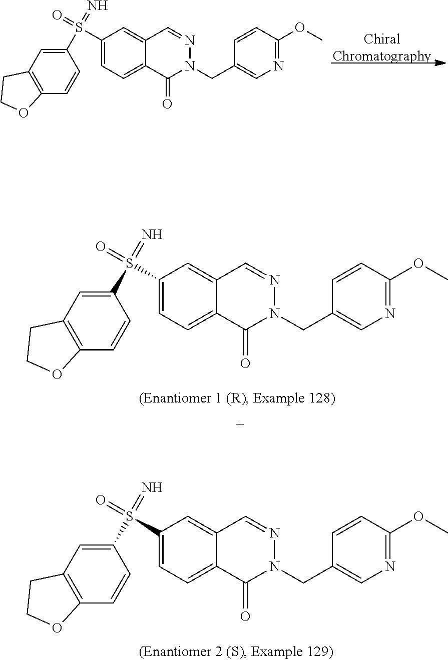

Racemic 6-(2,3-dihydrobenzofuran-5-sulfonimidoyl)-2-((6-methoxypyridin-3-yl)methyl)phthalazine-1(2H)-one (Example 127), was dissolved to 50 mg/mL in MeOH with sonication, filtered and was then separated by chiral SFC on a Waters prep 15 with UV detection by DAD at 210-400 nm, 40° C., 120 bar. The column was IA 10×250 mm, 5 um, flow rate 15 mL/min at 45% IPA (neutral), 55% CO₂. The clean fractions were pooled, rinsed with methanol and concentrated to dryness using a rocket evaporator at 40° C. The residues were re-dissolved in methanol transferred into final vials and evaporated on a Biotage V10. The samples were then further dried in a vacuum oven at 30° C./5 mbar overnight to afford (R)-6-(2,3-dihydrobenzofuran-5-sulfonimidoyl)-2-((6-methoxypyridin-3-yl)methyl)phthalazin-1(2H)-one (100% ee, RT: 2.88) and (S)-6-(2,3-dihydrobenzofuran-5-sulfonimidoyl)-2-((6-methoxypyridin-3-yl)methyl)phthalazin-1(2H)-one (100% ee, RT: 3.24) as cloudy glass.

Stereochemistry was arbitrary assigned.

Analytical methos: SFC using a Waters UPC2. The column was a IA 4.6×250, Sum, flow rate 4 mL/min-1 eluting with 60% IPA (0.1% Ammonia), 40% CO₂ at a wavelength 210-400 nm and BPR 120 Bar.

### Example 130—6-((2,3-dihydro-[1,4]dioxino[2,3-b]pyridin-7-yl)sulfonyl)-2-((6-methoxypyridin-3-yl)methyl)phthalazin-1(2H)-one

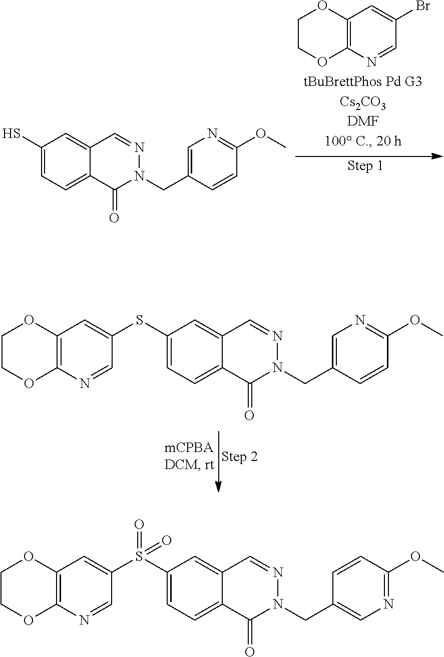 [SMILES: Brc1cnc2c(c1)OCCO2 ; COc1ccc(Cn2ncc3cc(S)ccc3c2=O)cn1 ; COc1ccc(Cn2ncc3cc(Sc4cnc5c(c4)OCCO5)ccc3c2=O)cn1 ; COc1ccc(Cn2ncc3cc(S(=O)(=O)c4cnc5c(c4)OCCO5)ccc3c2=O)cn1]

### Step 1

tBuBrettPhos Pd G3 (12 mg, 0.1 Eq, 14 mmol) was added to a degassed mixture of 6-mercapto-2-((6-methoxypyridin-3-yl)methyl)phthalazin-1(2H)-one (Intermediate 40, 0.065 g, 1 Eq, 0.22 mmol), 7-bromo-2,3-dihydro-[1,4]dioxino[2,3-b]pyridine (52 mg, 1.1 Eq, 0.24 mmol), cesium carbonate (0.11 g, 1.5 Eq, 0.33 mmol) in DMF (2.0 mL) and the resulting mixture was heated to 100° C. for overnight. After cooling to RT, the reaction mixture was diluted with water (10 mL) and extracted with DCM (2×20 mL). The organic phase was collected, dried (MgSO₄) and evaporated under reduced pressure. The crude product was purified by chromatography on silica gel to afford 6-((2,3-dihydro-[1,4]dioxino[2,3-b]pyridin-7-yl)thio)-2-((6-methoxypyridin-3-yl)methyl)phthalazin-1(2H)-one (0.080 g) as a slightly orange solid. MS (ES⁺): 435 (M+H)⁺

### Step 2

mCPBA (0.12 g, 77% Wt, 3.0 Eq, 0.55 mmol) was added to a stirred solution of 6-((2,3-dihydro-[1,4]dioxino[2,3-b]pyridin-7-yl)thio)-2-((6-methoxypyridin-3-yl)methyl)phthalazin-1(2H)-one (0.080 g, 1 Eq, 0.18 mmol) in DCM (2 mL) at RT and the reaction mixture was stirred for 1 hour, then diluted with DCM (5 mL) and washed with saturated NaHCO₃ (5 mL) and water (5 mL). The organic phase was then dried (phase separator) and evaporated under reduced pressure. The crude product was purified by chromatography on silica gel to afford the title compound (0.016 g) as an off white solid. ¹H NMR (400 MHz, DMSO-d6) δ 8.70 (d, 1H), 8.59 (s, 1H), 8.44-8.34 (m, 3H), 8.19 (d, 1H), 7.83 (d, 1H), 7.67 (dd, 1H), 6.76 (d, 1H), 5.27 (s, 2H), 4.53-4.46 (m, 2H), 4.35-4.27 (m, 2H), 3.80 (s, 3H). MS (ES⁺): 467 (M+H)⁺

### Example 131—2-((6-methoxypyridin-3-yl)methyl)-6-(phenylsulfonimidoyl)phthalazin-1(2H)-one

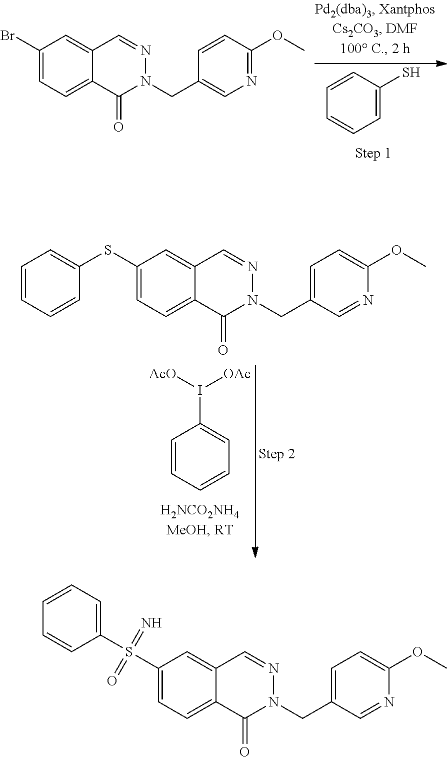

### Step 1

N₂ was bubbled through a mixture of 6-bromo-2-((6-methoxypyridin-3-yl)methyl)phthalazin-1(2H)-one (Intermediate 39, 75 mg, 1 Eq, 0.22 mmol), benzenethiol (24 mg, 22 μL, 1 Eq, 0.22 mmol) and cesium carbonate (0.11 g, 1.5 Eq, 0.32 mmol) in DMF (1 mL) for 5 minutes. Pd₂dba₃ (9.9 mg, 0.05 Eq, 11 μmol) and Xantphos (13 mg, 0.1 Eq, 22 μmol) were added and the mixture was stirred at 100° C. under N₂ for 3 h, then allowed to cool to RT. The mixture was poured into ice/water and the resulting precipitate was filtered, washing with water, then dried in a desiccator at 45° C. for 3 days to afford crude 2-((6-methoxypyridin-3-yl)methyl)-6-(phenylthio)phthalazin-1(2H)-one (63 mg). The product was used without further purification in the next step. MS (ES)⁺: 376 (M+H)⁺

### Step 2

Iodobenzene diacetate (0.25 g, 5 Eq, 0.76 mmol) was added to a mixture of 2-((6-methoxypyridin-3-yl)methyl)-6-(phenylthio)phthalazin-1(2H)-one (63 mg, 91% Wt, 1 Eq, 0.15 mmol) and ammonium carbamate (60 mg, 5 Eq, 0.76 mmol) in MeOH (1.5 mL) and the mixture was stirred overnight. Sat. NaHCO₃ and DCM were added and the layers separated through a phase separator. The organic layer was washed with brine, then absorbed on silica. The crude product was purified by chromatography on silica gel to afford the title compound (32 mg) as a white solid. ¹H NMR (DMSO-d6) δ: 8.64-8.59 (m, 2H), 8.38 (d, J=8.4 Hz, 1H), 8.30 (dd, J=8.4, 1.8 Hz, 1H), 8.18 (d, J=2.4 Hz, 1H), 8.04-7.98 (m, 2H), 7.70-7.53 (m, 4H), 6.75 (d, J=8.5, 0.7 Hz, 1H), 5.39 (s, 1H), 5.26 (s, 2H), 3.80 (s, 3H). MS (ES)⁺: 407 (M+H)⁺

### Example 132—(R)-2-((6-methoxypyridin-3-yl)methyl)-6-(phenylsulfonimidoyl)phthalazin-1(2H)-one; and

### Example 133—(S)-2-((6-methoxypyridin-3-yl)methyl)-6-(phenylsulfonimidoyl)phthalazin-1(2H)-one

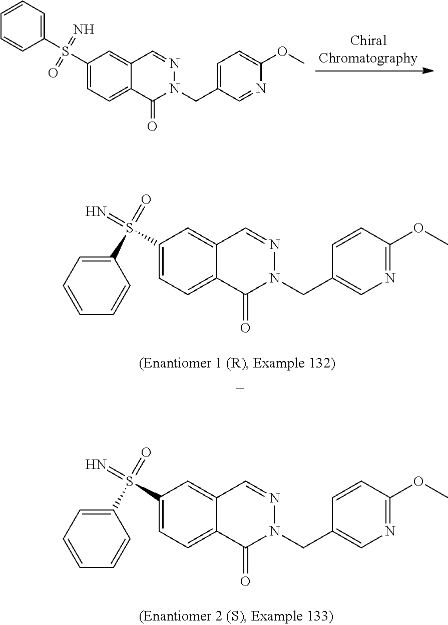 [SMILES: COc1ccc(Cn2ncc3cc(S(=N)(=O)c4ccccc4)ccc3c2=O)cn1 ; COc1ccc(Cn2ncc3cc([S@](=N)(=O)c4ccccc4)ccc3c2=O)cn1 ; COc1ccc(Cn2ncc3cc([S@@](=N)(=O)c4ccccc4)ccc3c2=O)cn1]

Racemic 2-((6-methoxypyridin-3-yl)methyl)-6-(phenylsulfonimidoyl)phthalazin-1(2H)-one (Example 131), as a racemic mixture, was dissolved to 9 mg/mL in DCM/MeOH with sonication, filtered and was then separated by chiral SFC on a Waters prep 15 with UV detection by DAD at 210-400 nm, 40° C., 120 bar. The column was Chiralpak IH 10×250 mm, 5 um, flow rate 15 mL/min at 40% MeOH, 60% CO₂. The clean fractions were pooled, rinsed with methanol/DCM and concentrated to dryness using a rotary evaporator. The residues were re-dissolved in methanol transferred into final vials and evaporated on a Biotage V10. The samples were then further dried in a vacuum oven at 30° C./5 mbar overnight to afford (R)-2-((6-methoxypyridin-3-yl)methyl)-6-(phenylsulfonimidoyl)phthalazin-1(2H)-one (99.5% ee, RT: 3.12 min.) and (S)-2-((6-methoxypyridin-3-yl)methyl)-6-(phenylsulfonimidoyl)phthalazin-1(2H)-one (98.8% ee, RT: 3.38 min.) as white solids. Stereochemistry was arbitrary assigned.

Analytical method: SFC using a Waters UPC2. The column was a Chiralpak IH 4.6×250, 5 um, flow rate 4 mL/min-1 eluting with 40% MeOH (0.1% Ammonia), 60% CO₂ at a wavelength 210-400 nm and BPR 120 Bar.

### Example 134—6-(4-(difluoromethoxy)phenylsulfonimidoyl)-2-((6-methoxypyridin-3-yl)methyl)phthalazin-1(2H)-one

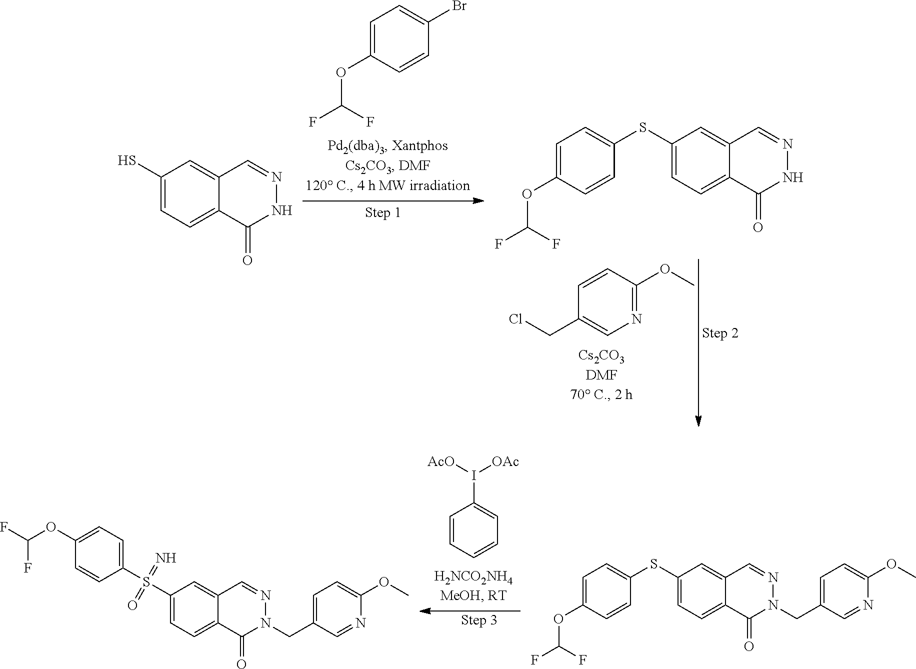

### Step 1

A mixture of 6-mercaptophthalazin-1(2H)-one (Intermediate 53, 100 mg, 1 Eq, 561 μmol), Pd₂(dba)₃ (25.7 mg, 0.05 Eq, 28.1 μmol), Xantphos (32.5 mg, 0.1 Eq, 56.1 μmol), 1-bromo-4-(difluoromethoxy)benzene (188 mg, 1.5 Eq, 842 μmol), and Cs₂CO₃ (366 mg, 2 Eq, 1.12 mmol) in DMF (4 mL) was heated to 120° C. under microwave irradiation for 4 h. After cooling to RT, the reaction mixture was diluted with water (20 mL) and extracted with DCM (2×20 mL). The organic phase was collected, dried (phase separator) and evaporated under reduced pressure. The crude product was purified by chromatography on silica gel to afford 6-((4-(difluoromethoxy)phenyl)thio)phthalazin-1(2H)-one (0.10 g) as a sticky orange solid. MS (ES⁺): 321 (M+H)⁺

### Step 2

Cesium carbonate (0.23 g, 2.5 Eq, 0.71 mmol) was added to a stirred solution of 6-((4-(difluoromethoxy)phenyl)thio)phthalazin-1(2H)-one (0.12 g, 76% Wt, 1 Eq, 0.28 mmol) in dry DMF (6 mL) and the reaction mixture was stirred for 5 minutes, then 5-(chloromethyl)-2-methoxypyridine, HCl (66 mg, 1.2 Eq, 0.34 mmol) was added. The resulting mixture was stirred at 70° C. for 2 h, then allowed to cool to RT. The reaction mixture was diluted with water (20 mL) and extracted with DCM (20 mL). The organic phase was collected, dried (phase separator) and evaporated under reduced pressure. The crude product was purified by chromatography on silica gel to afford 6-((4-(difluoromethoxy)phenyl)thio)-2-((6-methoxypyridin-3-yl)methyl)phthalazin-1(2H)-one (0.12 g) as a sticky orange oil. MS (ES⁺): 442 (M+H)⁺

### Step 3

Ammonium carbamate (82.8 mg, 5 Eq, 1.06 mmol) and Iodobenzene diacetate (341 mg, 5 Eq, 1.06 mmol) were added to a stirred solution of 6-((4-(difluoromethoxy)phenyl)thio)-2-((6-methoxypyridin-3-yl)methyl)phthalazin-1(2H)-one (120 mg, 78% Wt, 1 Eq, 212 μmol) in dry MeOH (6 mL) and the mixture was stirred for 3 h at RT. The reaction mixture was evaporated under reduced pressure and the residue purified by preparative HPLC (basic method) to afford the title compound (10 mg) as a clear white solid. ¹H NMR (DMSO-d6) δ: 8.62 (d, J=2.0 Hz, 2H), 8.38 (d, J=8.5 Hz, 1H), 8.30 (dd, J=8.5, 1.8 Hz, 1H), 8.18 (d, J=2.4 Hz, 1H), 8.09-8.01 (m, 2H), 7.67 (dd, J=8.5, 2.5 Hz, 1H), 7.39-7.30 (m, 2H), 7.35 (t, J=73.2 Hz, 1H), 6.75 (dd, J=8.5, 0.7 Hz, 1H), 5.46 (s, 1H), 5.26 (s, 2H), 3.80 (s, 3H). MS (ES⁺): 473 (M+H)⁺

### Example 135—(R)-6-(4-(difluoromethoxy)phenylsulfonimidoyl)-2-((6-methoxypyridin-3-yl)methyl)phthalazin-1(2H)-one; and

### Example 136—(S)-6-(4-(difluoromethoxy)phenylsulfonimidoyl)-2-((6-methoxypyridin-3-yl)methyl)phthalazin-1(2H)-one

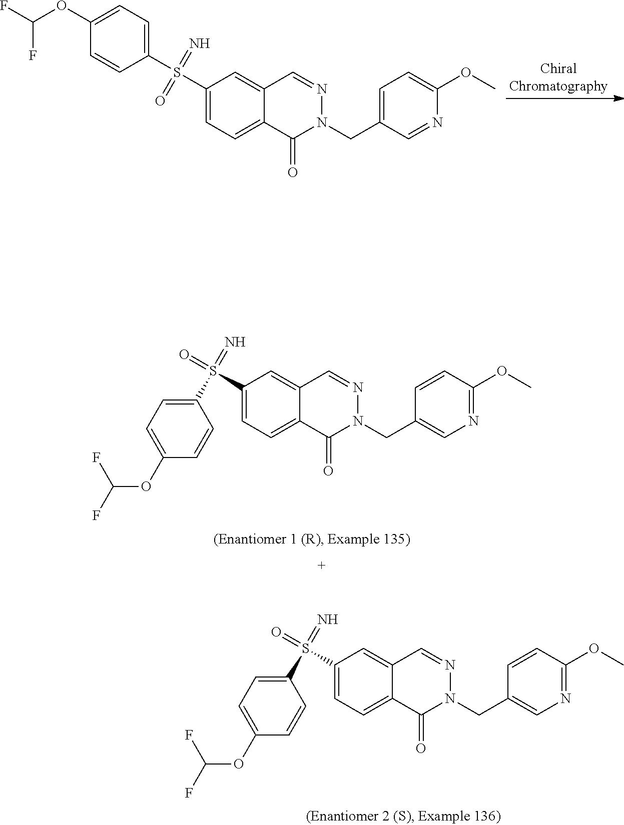 [SMILES: COc1ccc(Cn2ncc3cc(S(=N)(=O)c4ccc(OC(F)F)cc4)ccc3c2=O)cn1 ; COc1ccc(Cn2ncc3cc([S@](=N)(=O)c4ccc(OC(F)F)cc4)ccc3c2=O)cn1 ; COc1ccc(Cn2ncc3cc([S@@](=N)(=O)c4ccc(OC(F)F)cc4)ccc3c2=O)cn1]

Racemic 6-(4-(difluoromethoxy)phenylsulfonimidoyl)-2-((6-methoxypyridin-3-yl)methyl)phthalazin-1(2H)-one (Example 134), was dissolved to 7 mg/mL in DCM/MeOH with sonication, filtered and was then separated by chiral SFC on a Waters prep 15 with UV detection by DAD at 210-400 nm, 40° C., 120 bar. The column was Phenomenex Lux C4 10×250 mm, 5 um, flow rate 15 mL/min at 55% MeOH, 45% CO₂. The clean fractions were pooled, rinsed with methanol/DCM and concentrated to dryness using a rotary evaporator. The residues were re-dissolved in methanol/DCM transferred into final vials and evaporated on a Biotage V10. The samples were then further dried in a vacuum oven at 30° C./5 mbar over night to afford (R)-6-(4-(difluoromethoxy)phenylsulfonimidoyl)-2-((6-methoxypyridin-3-yl)methyl)phthalazin-1(2H)-one (100% ee, RT: 2.46 min.) and (S)-6-(4-(difluoromethoxy)phenylsulfonimidoyl)-2-((6-methoxypyridin-3-yl)methyl)phthalazin-1(2H)-one (100% ee, RT: 3.20 min.) as white solids.

Stereochemistry was arbitrary assigned.

Analytical method: SFC using a Waters UPC2. The column was a Chiralpak IA 4.6×250, 5 um, flow rate 4 mL/min-1 eluting with 60% MeOH (0.1% Ammonia), 40% CO₂ at a wavelength 210-400 nm and BPR 120 Bar.

### Example 137—6-(4-methoxyphenylsulfonimidoyl)-2-((6-methoxypyridin-3-yl)methyl)phthalazin-1(2H)-one

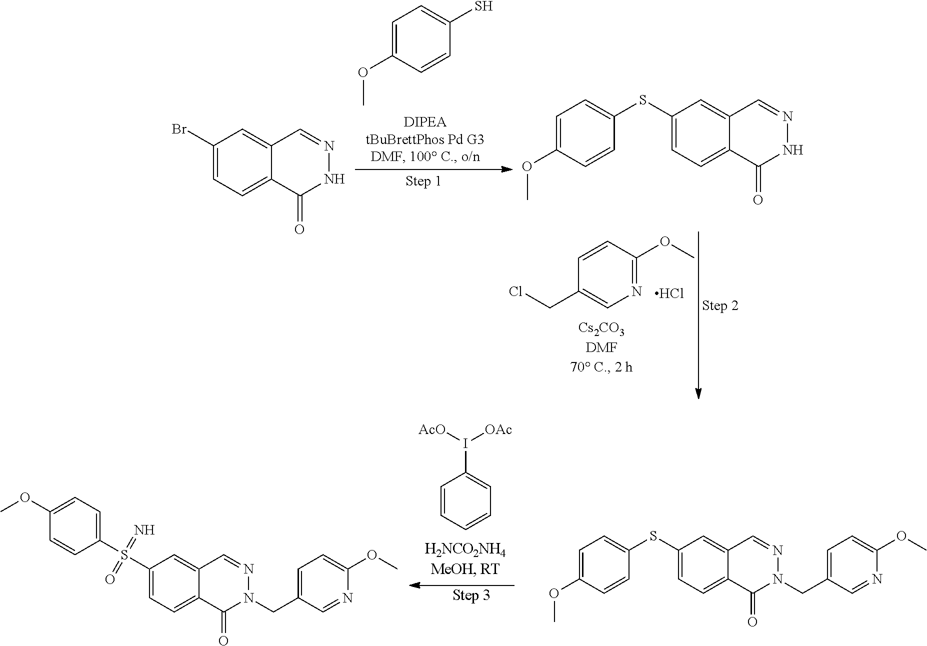

### Step 1

N₂ was bubbled through a mixture of 6-bromophthalazin-1(2H)-one (0.70 g, 1 Eq, 3.1 mmol), 4-methoxybenzenethiol (0.44 g, 0.38 mL, 1 Eq, 3.1 mmol) and DIPEA (0.80 g, 1.1 mL, 2 Eq, 6.2 mmol) in DMF (12 mL) and the mixture was stirred for 5 minutes. tBuBrettPhos Pd G3 (0.13 g, 0.05 Eq, 0.16 mmol) was added and the mixture was stirred at 100° C. under N₂ overnight, then allowed to cool to RT. The mixture was poured into ice and the resulting precipitate washed with water, then DCM and MTBE to give crude 6-((4-methoxyphenyl)thio)phthalazin-1(2H)-one (0.89 g) as an orange solid. MS (ES⁺): 285 (M+H)⁺

### Step 2

Cesium carbonate (0.54 g, 2.5 Eq, 1.7 mmol) was added to a stirred solution of 6-((4-methoxyphenyl)thio)phthalazin-1(2H)-one (0.20 g, 95% Wt, 1 Eq, 0.67 mmol) in dry DMF (6 mL) and the reaction mixture was stirred for 5 minutes. 5-(chloromethyl)-2-methoxypyridine, HCl (0.16 g, 1.2 Eq, 0.80 mmol) was added and the resulting mixture was stirred at 70° C. for 2 h. After cooling to RT, the reaction mixture was diluted with water (20 mL) and extracted with EtOAc (2×20 mL). The organic phase was collected, dried (MgSO₄) and evaporated under reduced pressure, giving 6-((4-methoxyphenyl)thio)-2-((6-methoxypyridin-3-yl)methyl)phthalazin-1(2H)-one (0.25 g) as a sticky orange solid. The product was used without further purification in the next step. MS (ES⁺): 406 (M+H)⁺

### Step 3

Ammonium carbamate (217 mg, 5 Eq, 2.77 mmol) and Iodobenzene diacetate (894 mg, 5 Eq, 2.77 mmol) were added to a stirred solution of 6-((4-methoxyphenyl)thio)-2-((6-methoxypyridin-3-yl)methyl)phthalazin-1(2H)-one (250 mg, 1 Eq, 555 μmol) in dry MeOH (6 mL) and the mixture was stirred for 3 h at RT, then it was diluted with water (30 mL). The resulting precipitate was filtered, washed with water and MTBE, giving the crude product. The crude product was purified by chromatography on silica gel to afford the title compound (60 mg) as a clear white solid. ¹H NMR (400 MHz, DMSO-d6) δ 8.61 (d, J=0.7 Hz, 1H), 8.57 (d, J=1.8 Hz, 1H), 8.36 (d, J=8.4 Hz, 1H), 8.26 (dd, J=8.4, 1.8 Hz, 1H), 8.18 (d, J=2.4 Hz, 1H), 7.96-7.87 (m, 2H), 7.66 (dd, J=8.5, 2.5 Hz, 1H), 7.13-7.05 (m, 2H), 6.75 (dd, J=8.5, 0.7 Hz, 1H), 5.26 (s, 2H), 5.23 (s, 1H), 3.80 (s, 3H), 3.80 (s, 3H). MS (ES⁺): 437 (M+H)⁺

### Example 138—(R)-6-(4-methoxyphenylsulfonimidoyl)-2-((6-methoxypyridin-3-yl)methyl)phthalazin-1(2H)-one; and

### Example 139—(S)-6-(4-methoxyphenylsulfonimidoyl)-2-((6-methoxypyridin-3-yl)methyl)phthalazin-1(2H)-one

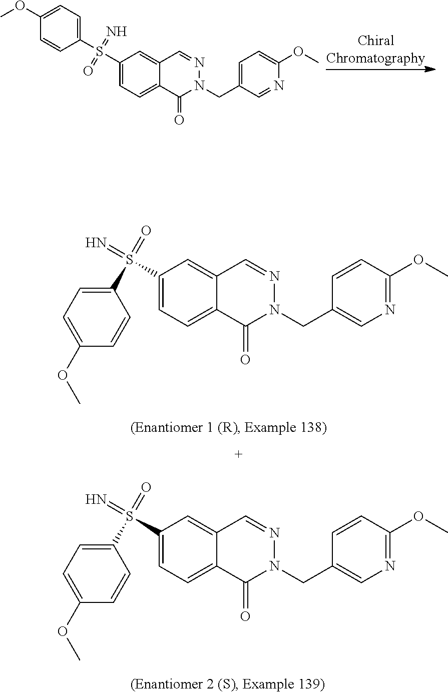

Racemic 6-(4-methoxyphenylsulfonimidoyl)-2-((6-methoxypyridin-3-yl)methyl)phthalazin-1(2H)-one (Example 137), was dissolved to 14.2 mg/mL in DMSO with sonication, filtered and was then separated by chiral LC on a Gilson UV directed prep with UV detection at 220 nm, 25° C. The column was an IA column 20×250 mm, Sum, flow rate 20 mL/min at 5% Water (0.1% Ammonia), 95% MeCN. The clean fractions were pooled, concentrated via rotary evaporation and then freeze dried. The dried compound was re-dissolved in methanol/DCM (1:1) transferred into final vials and evaporated on a Biotage V10. The samples were then further dried in a vacuum oven at 30° C./5 mbar overnight to afford (R)-6-(4-methoxyphenylsulfonimidoyl)-2-((6-methoxypyridin-3-yl)methyl)phthalazin-1(2H)-one (100% ee, RT: 4.88 min.) and (S)-6-(4-methoxyphenylsulfonimidoyl)-2-((6-methoxypyridin-3-yl)methyl)phthalazin-1(2H)-one (100% ee, RT: 8.80 min.) as white solids. Stereochemistry arbitrary assigned.

Analytical method: Agilent 1100. The column was an IA column 4.6×150 mm, 5 um, flow rate 1.0 mL/min-1 eluting with 10% Water (0.1% DEA), 90% MeCN at a wavelength 260+/−80 nm

### Example 140—6-((1H-pyrazol-3-yl)sulfonyl)-2-((6-methoxypyridin-3-yl)methyl)phthalazin-1(2H)-one

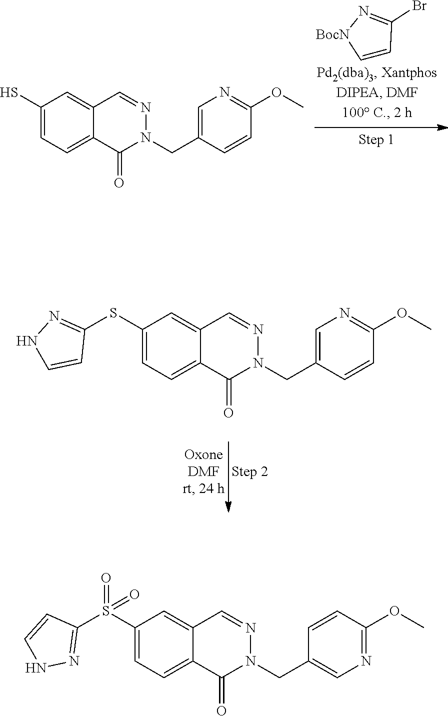 [SMILES: CC(C)(C)OC(=O)n1ccc(Br)n1 ; COc1ccc(Cn2ncc3cc(S)ccc3c2=O)cn1 ; COc1ccc(Cn2ncc3cc(Sc4cc[nH]n4)ccc3c2=O)cn1 ; COc1ccc(Cn2ncc3cc(S(=O)(=O)c4cc[nH]n4)ccc3c2=O)cn1]

### Step 1

A mixture of 6-mercapto-2-((6-methoxypyridin-3-yl)methyl)phthalazin-1(2H)-one (Intermediate 40, 0.16 g, 1 Eq, 0.53 mmol), Pd₂(dba)₃ (24 mg, 0.05 Eq, 27 μmol), Xantphos (31 mg, 0.1 Eq, 53 μmol), tert-butyl 3-bromo-1H-pyrazole-1-carboxylate (0.13 g, 1 Eq, 0.53 mmol), and DIPEA (0.17 g, 0.23 mL, 2.5 Eq, 1.3 mmol) in DMF (6 mL) was heated to 120° C. under microwave irradiation for 2 hours. After cooling to RT, the reaction was poured in ice/water and the resulting precipitate filtered and washed with MTBE (20 mL), giving the crude product as a dark orange oil. The crude product was purified by chromatography on silica gel to afford 6-((1H-pyrazol-3-yl)thio)-2-((6-methoxypyridin-3-yl)methyl)phthalazin-1(2H)-one (0.17 g) as a sticky orange solid. MS (ES⁺): 366 (M+H)⁺

### Step 2

OXONE® (592 mg, 3 Eq, 963 μmol) was added to a solution of 6-((1H-pyrazol-3-yl)thio)-2-((6-methoxypyridin-3-yl)methyl)phthalazin-1(2H)-one (170 mg, 69% Wt, 1 Eq, 321 μmol) in DMF (6 mL) and the reaction mixture was left to stir for 24 h at RT. The reaction mixture was diluted with DCM (10 mL) and absorbed on silica gel, then purified by chromatography on silica gel, followed by preparative HPLC (acidic method) to afford the title compound (30 mg) as a clear white solid. ¹H NMR (400 M Hz, DMSO-d6) b 13.97 (s, 1H), 8.66 (s, 1H), 8.62 (d, 1H), 8.45 (d, 1H), 8.28 (dd, 1H), 8.20 (d, 1H), 8.01 (d, 1H), 7.68 (dd, 1H), 6.94 (d, 1H), 6.76 (d, 1H), 5.27 (s, 2H), 3.81 (s, 3H). MS (ES⁺): 398 (M+H)₊

| Example | | |
|---|---|---|
| No. | Example Structure /Name | LCMS/¹H NMR data |
| 141 | 2-(3-aminobenzyl)-6- (phenylsulfonyl)phthalazin-1(2H)-one 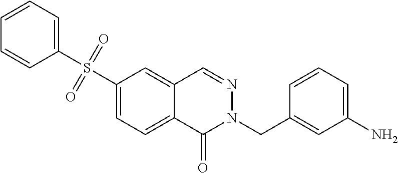 [SMILES: Nc1cccc(Cn2ncc3cc(S(=O)(=O)c4ccccc4)ccc3c2=O)c1] | MS (ES⁺): 392 (M + H)⁺ ¹H NMR (400 MHz, DMSO-d6) δ 8.70 (d, J = 1.8 Hz, 1H), 8.63 (s, 1H), 8.44 (d, J = 8.4 Hz, 1H), 8.31 (dd, J = 8.4, 1.9 Hz, 1H), 8.07- 8.00 (m, 2H), 7.79-7.70 (m, 1H), 7.70-7.62 (m, 2H), 6.97-6.88 (m, 1H), 6.45-6.39 (m, 3H), 5.17 (s, 2H), 5.02 (s, 2H). |
| 142 | 2-(3-aminobenzyl)-6-((4- methoxyphenyl)sulfonyl)phthalazin-1(2H)-one 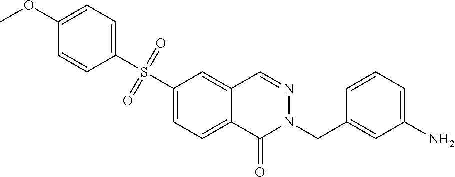 [SMILES: COc1ccc(S(=O)(=O)c2ccc3c(=O)n(Cc4cccc(N)c4)ncc3c2)cc1] | MS(ES⁺): 422 (M + H)⁺ 1H NMR (DMSO-d6) δ : 8.64 (d, 1H), 8.62 (d, 1H), 8.42 (d, 1H), 8.27 (dd, 1H), 8.00-7.91 (m, 2H), 7.21-7.13 (m, 2H), 6.96-6.88 (m, 1H), 6.46-6.38 (m, 3H), 5.17 (s, 2H), 5.02 (s, 2H), 3.83 (s, 3H). |
| 143 | 2-(3-aminobenzyl)-6-((2,3- dihydrobenzofuran-5- yl)sulfonyl)phthalazin-1(2H)-one 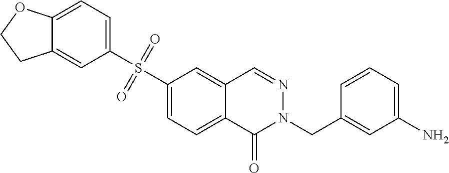 [SMILES: Nc1cccc(Cn2ncc3cc(S(=O)(=O)c4ccc5c(c4)CCO5)ccc3c2=O)c1] | MS(ES⁺): 434(M + H)⁺ ¹H NMR (DMSO-d6) δ: 8.63 (d, 1H), 8.62 (s, 1H), 8.42 (d, 1H), 8.27 (dd, 1H), 7.89-7.83 (m, 1H), 7.79 (dd, 1H), 6.98 (d, 1H), 6.96-6.88 (m, 1H), 6.45-6.38 (m, 3H), 5.17 (s, 2H), 5.02 (s, 2H), 4.64 (t, 2H), 3.24 (t, 2H). |
| 144 | 2-(3-aminobenzyl)-6-(pyridin-2- ylsulfonyl)phthalazin-1(2H)-one 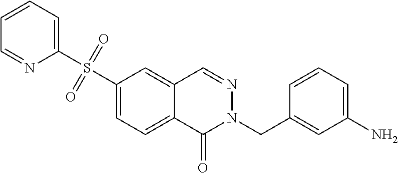 [SMILES: Nc1cccc(Cn2ncc3cc(S(=O)(=O)c4ccccn4)ccc3c2=O)c1] | MS(ES⁺): 393(M + H)⁺. ¹H NMR (DMSO-d6) δ: 8.73-8.69 (m, 2H), 8.68 (s, 1H), 8.47 (d, 1H), 8.36-8.27 (m, 2H), 8.20 (ddd, 1H), 7.73 (ddd, 1H), 6.93 (dd, 1H), 6.47- 6.39 (m, 3H), 5.18 (s, 2H), 5.03 (s, 2H). |
| 145 | 2-(3-aminobenzyl)-6-((1-methyl-1H- pyrazol-3-yl)sulfonyl)phthalazin-1(2H)-one 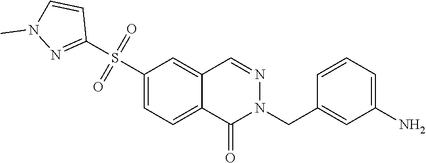 [SMILES: Cn1ccc(S(=O)(=O)c2ccc3c(=O)n(Cc4cccc(N)c4)ncc3c2)n1] | MS (ES⁺): 396 (M + H)⁺ ¹H NMR (400 MHz, DMSO-d6) δ 8.67 (s, 1H), 8.64 (d, J = 1.8 Hz, 1H), 8.46 (d, J = 8.4 Hz, 1H), 8.28 (dd, J = 8.4, 1.8 Hz, 1H), 7.97 (d, J = 2.4 Hz, 1H), 6.95 (d, J = 2.3 Hz, 1H), 6.92 (d, J = 8.0 Hz, 1H), 6.47- 6.39 (m, 3H), 5.18 (s, 2H), 5.03 (s, 2H), 3.91 (s, 3H). |
| 146 | 2-(3-aminobenzyl)-6-((5-methylthiophen- 2-yl)sulfonyl)phthalazin-1(2H)-one 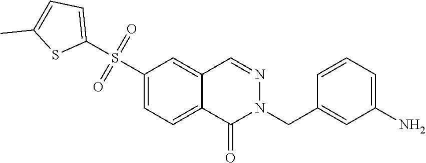 [SMILES: Cc1ccc(S(=O)(=O)c2ccc3c(=O)n(Cc4cccc(N)c4)ncc3c2)s1] | MS (ES⁺): 412 (M + H)⁺ ¹H NMR (DMSO-d6) δ: 8.66 (d, J = 1.9 Hz, 1H), 8.65 (s, 1H), 8.45 (d, J = 8.4 Hz, 1H), 8.31 (dd, J = 8.5, 1.9 Hz, 1H), 7.78 (d, J = 3.8 Hz, 1H), 7.01 (dd, J = 3.9, 1.2 Hz, 1H), 6.94 (dd, J = 8.9, 6.9 Hz, 1H), 6.50-6.41 (m, 3H), 5.18 (s, 3H). |
| 147 | 6-((4-fluorophenyl)sulfonyl)-2-((6- methoxypyridin-3-yl)methyl)phthalazin-1(2H)-one 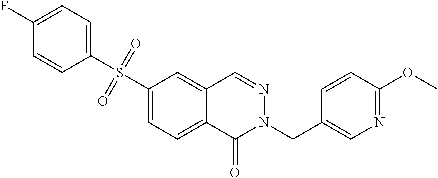 [SMILES: COc1ccc(Cn2ncc3cc(S(=O)(=O)c4ccc(F)cc4)ccc3c2=O)cn1] | MS (ES⁺): 426 (M + H)⁺ ¹H NMR (400 MHz, DMSO-d6) δ 8.68 (d, J = 1.8 Hz, 1H), 8.61 (d, J = 0.7 Hz, 1H), 8.43 (d, J = 8.5 Hz, 1H), 8.31 (dd, J = 8.4, 1.9 Hz, 1H), 8.19 (d, J = 2.4 Hz, 1H), 8.13-8.07 (m, 2H), 7.67 (dd, J = 8.6, 2.5 Hz, 1H), 7.55-7.46 (m, 2H), 6.76 (dd, J = 8.5, 0.7 Hz, 1H), 5.27 (s, 2H), 3.80 (s, 3H). |
| 148 | 2-((6-methoxypyridin-3-yl)methyl)-6-(3- methylisothiazol-5-ylsulfonyl)phthalazin-1(2H)-one 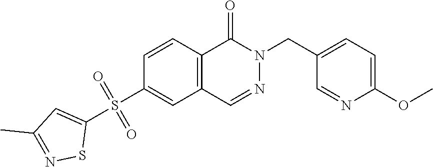 | MS (ES⁺): 429.0 (M + H)⁺ ¹H NMR (400 MHz, DMSO-d6) δ: 8.78 (d, J = 1.6 Hz, 1H), 8.66 (s, 1H), 8.49(d, J = 8.4 Hz, 1H), 8.41 (dd, J1= 1.6 Hz, J2 = 8.4 Hz, 1H), 8.20(d, J = 2.0 Hz, 1H), 7.97 (s, 1H), 7.69 (dd, J1 = 2.4 Hz, J2 = 8.4 Hz, 1H), 6.77 (d, J = 8.4 Hz, 1H), 5.29 (s, 2H), 3.81 (s, 3H), 2.45 (s, 3H). |
| 149 | 2-(3-aminobenzyl)-6-((6-methoxypyridin- 3-yl)sulfonyl)phthalazin-1(2H)-one 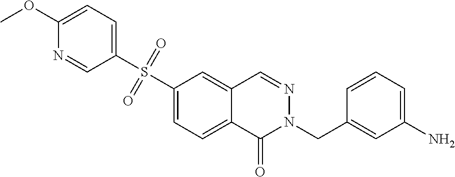 [SMILES: COc1ccc(S(=O)(=O)c2ccc3c(=O)n(Cc4cccc(N)c4)ncc3c2)cn1] | MS (ES⁺): 423 (M + H)⁺ ¹H NMR (400 MHz, DMSO-d6) δ 8.87 (d, J = 2.6 Hz, 1H), 8.70 (d, J = 1.8 Hz, 1H), 8.61 (s, 1H), 8.44 (d, J = 8.5 Hz, 1H), 8.35 (dd, J = 8.5, 1.9 Hz, 1H), 8.23 (dd, J = 8.9, 2.6 Hz, 1H), 7.04 (d, J = 8.9 Hz, 1H), 6.92 (t, J = 7.3 Hz, 1H), 6.46-6.40 (m, 3H), 5.17 (s, 2H), 5.02 (s, 2H), 3.94 (s, 3H). |
| 150 | 2-((6-methoxypyridin-3-yl)methyl)-6- tosylphthalazin-1(2H)-one 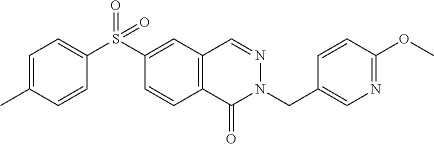 | MS (ES⁺): 422 (M + H)⁺ ¹H NMR (400 MHz, DMSO-d6) δ 8.65 (d, J = 1.8 Hz, 1H), 8.61 (s, 1H), 8.41 (d, J = 8.4 Hz, 1H), 8.28 (dd, J = 8.4, 1.9 Hz, 1H), 8.19 (d, J = 2.4 Hz, 1H), 7.92-7.87 (m, 2H), 7.67 (dd, J = 8.5, 2.5 Hz, 1H), 7.46 (d, J = 8.1 Hz, 2H), 6.76 (d, J = 8.6 Hz, 1H), 5.26 (s, 2H), 3.80 (s, 3H), 2.37 (s, 3H). |
| 151 | 2-(3-aminobenzyl)-6-((2-mathylthiazol-4- yl)sulfonyl)phthalazin-1(2H)-one 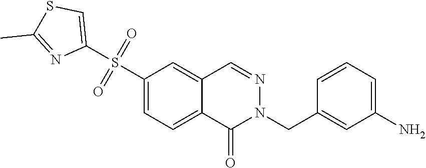 | MS (ES⁺): 436 (M + H)⁺ ¹H NMR (400 MHz, DMSO-d6) δ 8.79 (d, J = 1.9 Hz, 1H), 8.66 (s, 1H), 8.50 (d, J = 8.5 Hz, 1H), 8.42 (dd, J = 8.4, 1.9 Hz, 1H), 7.97 (s, 1H), 6.93 (t, J = 7.6 Hz, 1H), 6.48- 6.40 (m, 3H), 5.18 (s, 2H), 5.03 (s, 2H), 2.46 (s, 3H). |
| 152 | 2-(3-aminobenzyl)-6-(benzo[d][1,3]dioxol- 5-ylsulfonyl)phthalazin-1(2H)-one 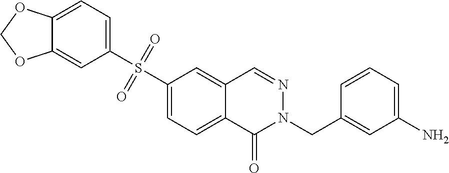 [SMILES: Nc1cccc(Cn2ncc3cc(S(=O)(=O)c4ccc5c(c4)OCO5)ccc3c2=O)c1] | MS (ES⁺): 436 (M + H)⁺ ¹H NMR (400 MHz, DMSO-d6) δ 8.66 (d, J = 1.8 Hz, 1H), 8.61 (s, 1H), 8.42 (d, J = 8.4 Hz, 1H), 8.30 (dd, J = 8.4, 1.9 Hz, 1H), 7.59 (dd, J = 8.2, 1.9 Hz, 1H), 7.52 (d, J = 1.9 Hz, 1H), 7.15 (d, J = 8.2 Hz, 1H), 6.92 (t, J = 6.8 Hz, 1H), 6.47 - 6.38 (m, 3H), 6.17 (s, 2H), 5.17 (s, 2H), 5.02 (s, 2H). |
| 153 | 6-(4-(difluoromethoxy)phenylsulfonyl)-2- ((6-methoxypyridin-3-yl)methyl)phthalazin-1(2H)-one 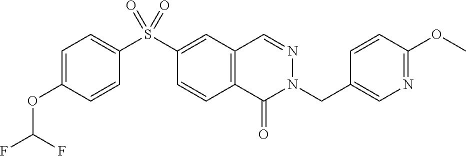 [SMILES: COc1ccc(Cn2ncc3cc(S(=O)(=O)c4ccc(OC(F)F)cc4)ccc3c2=O)cn1] | MS (ES)⁺: 474.0 (M + H)⁺ ¹H NMR (400 MHz, DMSO-d6) δ: 8.69 (d, J = 1.2 Hz, 1H), 8.62 (s, 1H), 8.43 (d, J = 8.4 Hz, 1H), 8.31 (dd, J = 8.4, 1.6 Hz, 1H), 8.19 (d, J = 2.4 Hz, 1H), 8.10 (dd, J = 7.2, 2.0 Hz, 2H), 7.68 (dd, J = 8.4, 2.4 Hz, 1H), 7.43 (t, J = 65.6 Hz, 1H), 7.41 (d, J = 1.2 Hz, 2H), 6.76 (d, J = 8.4 Hz, 1H), 5.27 (s, 2H), 3.81 (s, 3H). |
| 154 | 6-((3-chlorophenyl)sulfonyl)-2-((6- methoxypyridin-3-yl)methyl)phthalazin-1(2H)-one 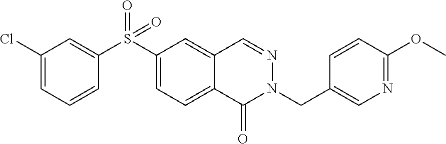 [SMILES: COc1ccc(Cn2ncc3cc(S(=O)(=O)c4cccc(Cl)c4)ccc3c2=O)cn1] | MS (ES⁺): 442/444 (M + H)⁺ ¹H NMR (400 MHz, DMSO-d6) δ 8.73 (s, 1H), 8.61 (s, 1H), 8.44 (d, J = 8.5 Hz, 1H), 8.40-8.32 (m, 1H), 8.19 (s, 1H), 8.08 (s, 1H), 7.99 (d, J = 7.9 Hz, 1H), 7.87- 7.78 (m, 1H), 7.75-7.61 (m, 2H), 6.76 (d, J = 8.6 Hz, 1H), 5.27 (s, 2H), 3.81 (s, 3H). |
| 155 | 6-((4-chlorophenyl)sulfonyl)-2-((6- methoxypyridin-3-yl)methyl)phthalazin-1(2H)-one 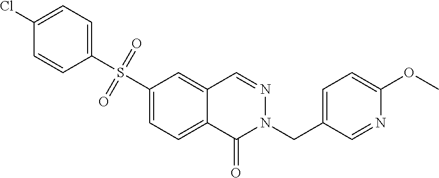 [SMILES: COc1ccc(Cn2ncc3cc(S(=O)(=O)c4ccc(Cl)cc4)ccc3c2=O)cn1] | MS (ES⁺): 442/444 (M + H)⁺ ¹H NMR (DMSO-d6) δ 8.68 (d, J = 1.8 Hz, 1H), 8.61 (s, 1H), 8.43 (d, J = 8.5 Hz, 1H), 8.31 (dd, J = 8.5, 1.9 Hz, 1H), 8.19 (d, J = 2.4 Hz, 1H), 8.06-8.00 (m, 2H), 7.77-7.71 (m, 2H), 7.67 (dd, J = 8.6, 2.5 Hz, 1H), 6.76 (d, J = 8.6 Hz, 1H), 5.27 (s, 2H), 3.80 (s, 3H). |
| 156 | 6-((2,2-difluorobenzo[d]][1,3]dioxol-5- yl)sulfonyl)-2-((6-methoxypyridin-3-yl)methyl)phthalazin-1(2H)-one 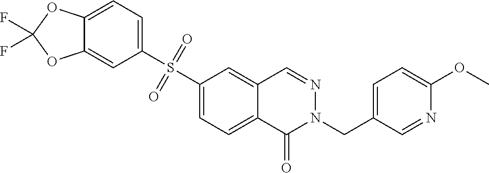 [SMILES: COc1ccc(Cn2ncc3cc(S(=O)(=O)c4ccc5c(c4)OC(F)(F)O5)ccc3c2=O)cn1] | MS (ES⁺): 488 (M + H)⁺ ¹H NMR (DMSO-d6) δ 8.68 (d, J = 1.8 Hz, 1H), 8.60 (s, 1H), 8.43 (d, J = 8.4 Hz, 1H), 8.33 (dd, J = 8.4, 1.9 Hz, 1H), 8.19 (d, J = 2.5 Hz, 1H), 8.15 (d, J = 1.9 Hz, 1H), 7.95 (dd, J = 8.5, 1.9 Hz, 1H), 7.72-7.64 (m, 2H), 6.76 (d, J = 8.5 Hz, 1H), 5.27 (s, 2H), 3.80 (s, 3H). |
| 157 | 6-((3-fluorophenyl)sulfonyl)-2-((6- methoxypyridin-3-yl)methyl)phthalazin-1(2H)-one 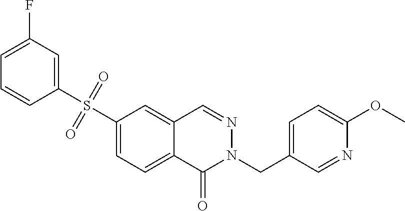 [SMILES: COc1ccc(Cn2ncc3cc(S(=O)(=O)c4cccc(F)c4)ccc3c2=O)cn1] | MS (ES⁺): 426 (M + H)⁺ ¹H NMR (DMSO-d6) δ 8.71 (d, J = 1.8 Hz, 1H), 8.61 (s, 1H), 8.43 (d, J = 8.4 Hz, 1H), 8.35 (d, J = 8.6 Hz, 1H), 8.19 (s, 1H), 7.89 (dd, J = 12.2, 8.4 Hz, 2H), 7.77-7.56 (m, 3H), 6.76 (d, J = 8.5 Hz, 1H), 5.27 (s, 2H), 3.80 (s, 3H). |
| 158 | 2-((6-methoxypyridin-3-yl)methyl)-6-((1- methyl-1H-pyrazol-3-yl)sulfonyl)phthalazin-1(2H)-one 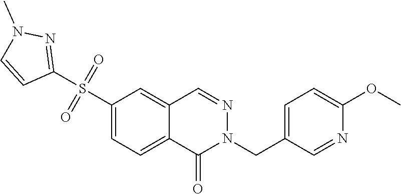 [SMILES: COc1ccc(Cn2ncc3cc(S(=O)(=O)c4ccn(C)n4)ccc3c2=O)cn1] | MS (ES⁺): 412 (M + H)⁺ ¹H NMR (400 MHz, DMSO-d6) δ 8.66 (s, 1H), 8.62 (d, J = 1.8 Hz, 1H), 8.45 (d, J = 8.4 Hz, 1H), 8.27 (dd, J = 8.4, 1.8 Hz, 1H), 8.20 (d, J = 2.5 Hz, 1H), 7.96 (d, J = 2.4 Hz, 1H), 7.69 (dd, J = 8.5, 2.5 Hz, 1H), 6.94 (d, J = 2.4 Hz, 1H), 6.76 (d, J = 8.5 Hz, 1H), 5.28 (s, 2H), 3.90 (s, 3H), 3.81 (s, 3H). |
| 159 | 2-((6-methoxypyridin-3-yl)methyl)-6-(4- methylpyridin-2-ylsulfonyl)phthalazin-1(2H)-one 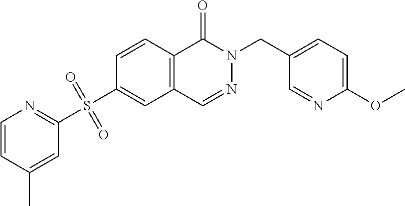 [SMILES: COc1ccc(Cn2ncc3cc(S(=O)(=O)c4cc(C)ccn4)ccc3c2=O)cn1] | MS (ES⁺): 423.0 (M + H)⁺ ¹H NMR (400 MHz, DMSO-d6) δ: 8.6 (d, J = 2.0 Hz, 2H), 8.51 (d, J = 4.8 Hz, 1H), 8.43 (d, J = 8.4 Hz, 1H), 8.27 (dd, J = 8.4, 1.6 Hz, 1H), 8.17 (d, J = 2.0 Hz, 1H), 8.13 (s, 1H), 7.66 (dd, J = 8.8, 2.4 Hz, 1H), 7.52 (d, J = 4.8 Hz, 1H), 6.74 (d, J = 8.8 Hz, 1H), 5.25 (s, 2H), 3.78 (s, 3H), 2.45 (s, 3H). |
| 160 | 6-(6-methoxypyridin-3-yl)methyl)phthalazin-1(2H)-onemethoxypyridin-2-ylsulfonyl)-2-((6- 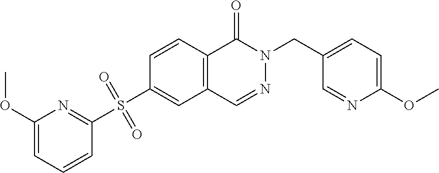 [SMILES: COc1ccc(Cn2ncc3cc(S(=O)(=O)c4cccc(OC)n4)ccc3c2=O)cn1] | MS (ES⁺): 439.0 (M + H)⁺ ¹H NMR (400 MHz, DMSO-d6) δ: 8.66 (d, J = 1.2 Hz, 1H), 8.65 (s, 1H), 8.45 (d, J = 8.4 Hz, 1H), 8.34 (dd, J = 8.4, 2.0 Hz, 1H), 8.17 (d, J = 2.4 Hz, 1H), 8.00 (t, J = 8.8 Hz, 1H), 7.82 (d, J = 7.2 Hz, 1H), 7.66 (dd, J = 8.8, 2.4 Hz, 1H), 7.10 (d, J = 8.4 Hz, 1H), 6.73 (d, J = 8.4 Hz, 1H), 5.24 (s, 2H), 3.77 (s, 3H), 3.73 (s, 3H). |
| 161 | 3-(2-((6-methoxypyridin-3-yl)methyl)-1- oxo-1,2-dihydrophthalazin-6-ylsulfonyl)benzamide 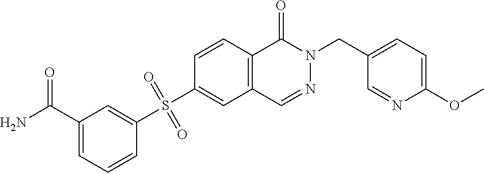 [SMILES: COc1ccc(Cn2ncc3cc(S(=O)(=O)c4cccc(C(N)=O)c4)ccc3c2=O)cn1] | MS (ES⁺): 451.0 (M + H)⁺ ¹H NMR (400 MHz, DMSO-d6) δ: 8.70 (s, 2H), 8.63 (s, 1H), 8.46-8.42 (m, 2H), 8.36-8.28 (m, 2H), 8.19- 8.15 (m, 3H), 7.75 (t, J = 8.0 Hz, 1H), 7.70-7.65 (m, 2H), 6.75 (d, J = 7.6 Hz 1H), 5.26 (s, 2H), 3.79 (s, 3H). |
| 162 | 6-(2-fluorophenylsulfonyl)-2-((6- methoxypyridin-3-yl)methyl)phthalazin-1(2H)-one 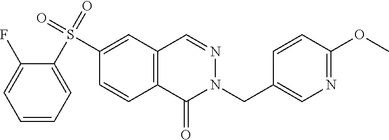 [SMILES: COc1ccc(Cn2ncc3cc(S(=O)(=O)c4ccccc4F)ccc3c2=O)cn1] | MS (ES⁺): 426.0 (M + H)⁺ ¹H NMR (400 MHz, DMSO-d6) δ: 8.65 (s, 2H), 8.44 (d, J = 8.4 Hz, 1H), 8.25 (dd, J = 8.8, 2.4 Hz, 1H), 8.17 (d, J = 2.4 Hz, 1H), 8.11 (td, J = 7.6, 1.6 Hz, 1H), 7.84-7.73 (m, 1H), 7.66 (dd, J = 8.4, 1.6 Hz, 1H), 7.52 (td, J = 7.6, 0.8 Hz, 1H), 7.42 (t, J = 1.6 Hz, 1H), 6.73 (d, J = 8.4 Hz, 1H), 5.25 (s, 2H), 3.78 (s, 3H). |
| 163 | 6-(2-chlorophenylsulfonyl)-2-((6- methoxypyridin-3-yl)methyl)phthalazin-1(2H)-one 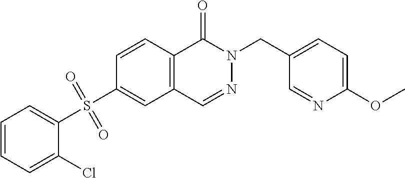 [SMILES: COc1ccc(Cn2ncc3cc(S(=O)(=O)c4ccccc4Cl)ccc3c2=O)cn1] | MS (ES⁺): 442.0 (M + H)⁺ ¹H NMR (400 MHz, DMSO-d6) δ: 8.67-8.65 (m, 2H), 8.61 (s, 1H), 8.44 (d, J = 8.4 Hz, 1H), 8.37 (d, J = 8.0 Hz, 1H), 8.24-8.20 (m, 1H), 6.757.80-7.65(m, 1H), 6.77(d, J = 8.8 Hz, 1H), 5.27 (s, 2H), 3.80 (s, 3H). |
| 164 | 6-(2-methoxyphenylsulfonyl)-2-((6- methoxypyridin-3-yl)methyl)phthalazin-1(2H)-one 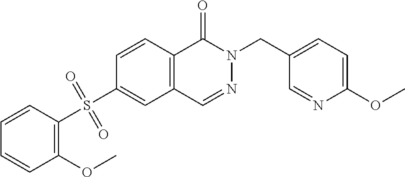 [SMILES: COc1ccc(Cn2ncc3cc(S(=O)(=O)c4ccccc4OC)ccc3c2=O)cn1] | MS (ES⁺): 438.0 (M + H)⁺ ¹H NMR (400 MHz, DMSO-d6) δ: 8.68 (s, 1H), 8.59 (s, 1H), 8.42 (d, J = 8.4 Hz, 1H), 8.24-8.20 (m, 2H), 8.07 (d, J = 7.6 Hz, 1H),7.72-6.68 (m, 2H), 7.23 (t, J = 7.2 Hz, 1H), 7.17 (d, J = 8.0 Hz, 1H), 6.77 (d, J = 8.4 Hz, 1H) , 5.27 (s, 2H), 3.80 (s, 3H), 3.70(s, 3H). |
| 165 | 6-(4-methoxypyridin-2-ylsulfonyl)-2-((6- methoxypyridin-3-yl)methyl)phthalazin-1(2H)-one 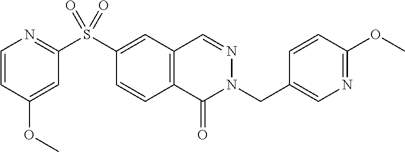 [SMILES: COc1ccnc(S(=O)(=O)c2ccc3c(=O)n(Cc4ccc(OC)nc4)ncc3c2)c1] | MS (ES⁺): 439.0 (M + H)⁺ ¹H NMR (400 MHz, DMSO-d6) δ: 8.65 (d, J = 1.6 Hz, 1H), 8.63 (s, 1H), 8.46 (d, J = 5.6 Hz, 1H), 8.43 (d, J = 8.4 1H), 8.29 (dd, J = 8.4, 1.6 Hz, 1H), 8.17 (d, J = 2.0 Hz, 1H), 7.75 (d, J = 2.4 Hz, 1H), 7.66 (dd, J = 8.8, 2.4 Hz, 1H), 7.23 (dd, J = 5.6, 2.4 Hz, 1H), 6.74 (d, J = 8.4 Hz, 1H), 5.25 (s, 2H), 3.94 (s, 3H), 3.78 (s, 3H). |
| 166 | 6-((2,2-dimethyl-2,3-dihydrobenzofuran-5- yl)sulfonyl)-2-((6-methoxypyridin-3-yl)methyl)phthalazin-1(2H)-one 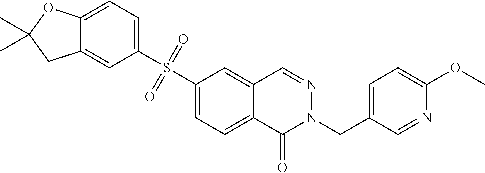 | MS (ES⁺): 478(M + H)⁺ ¹H NMR (DMSO-d6) δ: 8.66 - 8.59 (m, 2H), 8.41 (d, J = 8.4 Hz, 1H), 8.26 (dd, J = 8.5, 1.9 Hz, 1H), 8.20 (d, J = 2.4 Hz, 1H), 7.81 (d, J = 1.9 Hz, 1H), 7.78 (dd, J = 8.4, 2.2 Hz, 1H), 7.67 (dd, J = 8.5, 2.5 Hz, 1H), 6.92 (d, J = 8.5 Hz, 1H), 6.76 (d, J = 8.6 Hz, 1H), 5.27 (s, 2H), 3.80 (s, 3H), 3.07 (d, J = 4.8 Hz, 2H), 1.40 (s, 6H). |
| 167 | 2-((6-methoxypyridin-3-yl)methyl)-6-((2- methyl-2,3-dihydrobenzofuran-5-yl)sulfonyl)phthalazin-1(2H)-one 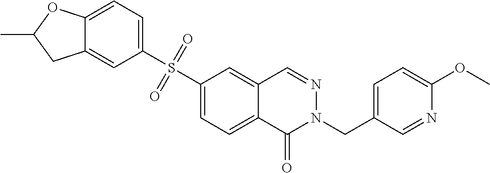 | MS (ES⁺): 464(M + H)⁺ ¹H NMR (DMSO-d6) δ: 8.63-8.58 (m, 2H), 8.41 (d, J = 8.4 Hz, 1H), 8.26 (dd, J = 8.4, 1.9 Hz, 1H), 8.19 (d, J = 2.4 Hz, 1H), 7.83-7.80 (m, 1H), 7.78 (dd, J = 8.5, 2.2 Hz, 1H), 7.67 (dd, J = 8.5, 2.5 Hz, 1H), 6.94 (d, J = 8.4 Hz, 1H), 6.76 (d, J = 8.7 Hz, 1H), 5.27 (s, 2H), 5.10 - 4.98 (m, 1H), 3.80 (s, 3H), 3.37 (dd, J = 16.3, 9.0 Hz, 1H), 2.83 (dd, J = 16.3, 7.5 Hz, 1H), 1.37 (d, J = 6.3 Hz, 3H). |
| 168 | 2-((6-methoxypyridin-3-yl)methyl)-6-((1- methyl-1H-pyrazol-4-yl)sulfonyl)phthalazin-1(2H)-one 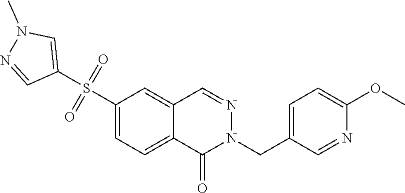 [SMILES: COc1ccc(Cn2ncc3cc(S(=O)(=O)c4cnn(C)c4)ccc3c2=O)cn1] | MS (ES⁺): 412 (M + H)⁺ ¹H NMR (DMSO-d6) δ: 8.62 (s, 1H), 8.59 (d, J = 1.8 Hz, 1H), 8.54 (s, 1H), 8.43 (d, J = 8.4 Hz, 1H), 8.28 (dd, J = 8.4, 1.9 Hz, 1H), 8.19 (d, J = 2.4 Hz, 1H), 8.00 (d, J = 0.8 Hz, 1H), 7.68 (dd, J = 8.6, 2.5 Hz, 1H), 6.77 (d, J = 8.5, 0.7 Hz, 1H), 5.28 (s, 2H), 3.87 (s, 3H), 3.81 (s, 3H). |
| 169 | 2-((6-methoxypyridin-3-yl)methyl)-6-(6- methylpyridin-2-ylsulfonyl)phthalazin-1(2H)-one 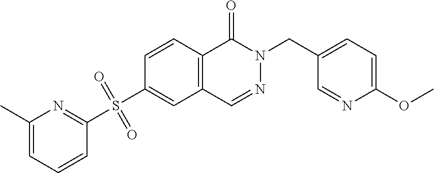 | MS (ES⁺): 423.0 (M + H)⁺ ¹H NMR (400 MHz, DMSO-d6) δ: 8.65 (d, J = 1.6 Hz, 2H), 8.43 (d, J = 8.4 Hz, 1H), 8.28 (dd, J = 8.4, 2.0 Hz, 1H), 8.17 (d, J = 2.0 Hz, 1H), 8.08-8.02 (m, 2H), 7.66 (dd, J = 8.8, 2.4 Hz, 1H), 7.55 (dd, J = 7.2, 1.2 Hz, 1H), 6.74 (d, J = 8.4 Hz, 1H), 5.25 (s, 2H), 3.78 (s, 3H), 2.43 (s, 3H) |
| 170 | 6-(3-(difluoromethoxy)phenylsulfonyl)-2- ((6-methoxypyridin-3-yl)methyl)phthalazin-1(2H)-one 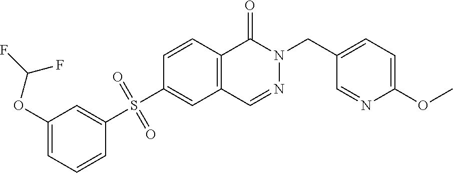 [SMILES: COc1ccc(Cn2ncc3cc(S(=O)(=O)c4cccc(OC(F)F)c4)ccc3c2=O)cn1] | MS (ES⁺): 474.0 (M + H)⁺ ¹H NMR (400 MHz, DMSO-d6) δ: 8.73 (d, J = 1.6 Hz, 1H), 8.63 (s, 1H), 8.44 (d, J = 8.4 Hz, 1H), 8.36 (dd, J = 8.8, 2.0 Hz, 1H), 8.20 (d, J = 2.0 Hz, 1H), 7.90 (d, J = 8.8 Hz, 1H), 7.81 (d, J = 1.6 Hz, 1H), 7.73 (t, J = 8.0 Hz, 1H), 7.68 (dd, J = 8.8, 2.4 Hz, 1H), 7.58 (d, J = 3.2 Hz, 1H), 7.40 (t, J = 67.6 Hz, 1H) 6.76 (d, J = 8.4 Hz, 1H), 5.28 (s, 2H), 3.81 (s, 3H). |
| 171 | 2-((6-methoxypyridin-3-yl)methyl)-6-(5- methylthiophen-2-ylsulfonyl)phthalazin-1(2H)-one 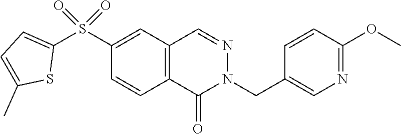 [SMILES: COc1ccc(Cn2ncc3cc(S(=O)(=O)c4ccc(C)s4)ccc3c2=O)cn1] | MS (ES⁺): 427.8 (M + H)⁺ ¹H NMR (400 MHz, DMSO-d6) δ: 8.64 (s, 2H), 8.44 (d, J = 8.8 Hz, 1H), 8.30 (dd, J1 = 1.6 Hz, J2 = 8.4 Hz, 1H), 8.19 (s, 1H), 7.78 (d, J = 4.0 Hz, 1H), 7.68 (dd, J1 = 2.4 Hz, J2 = 8.4 Hz, 1H), 7.01 (d, J = 3.2 Hz, 1H), 6.76 (d, J = 8.4 Hz, 1H), 5.27 (s, 2H), 3.80 (s, 3H). |
| 172 | 6-(5-methoxypyridin-2-ylsulfonyl)-2-((6- methoxypyridin-3-yl)methyl)phthalazin-1(2H)-one 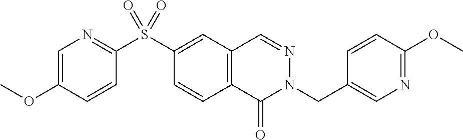 | MS (ES⁺): 439.0 (M + H)⁺ ¹H NMR (400 MHz, DMSO-d6) δ: 8.66 (s, 1H), 8.65 (d, J = 1.6 Hz, 1H), 8.45 (d, J = 8.4 Hz, 1H), 8.39 (d, J = 2.8 Hz, 1H), 8.29 (d, J = 2.0 Hz, 1H), 8.26 (d, J = 8.8 Hz, 1H), 8.20 (dd, J = 2.0 Hz, 1H), 7.68 (dd, J = 8.8, 2.8 Hz, 2H), 6.77 (d, J = 8.8 Hz, 1H), 5.27 (s, 2H), 3.90 (s, 3H), 3.81 (s, 3H). |
| 173 | 6-(2-(difluoromethyl)thiazol-5-ylsulfonyl)- 2-((6-methoxypyridin-3-yl)methyl)phthalazin-1(2H)-one 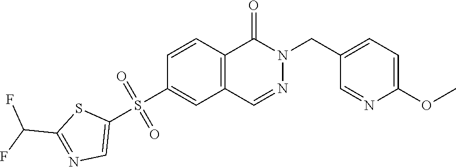 [SMILES: COc1ccc(Cn2ncc3cc(S(=O)(=O)c4cnc(C(F)F)s4)ccc3c2=O)cn1] | MS (ES⁺): 464.8 (M + H)⁺ ¹H NMR (400 MHz, DMSO-d6) δ: 8.83 (d, J = 1.6 Hz, 1H), 8.78 (d, J = 1.6 Hz, 1H), 8.65 (s, 1H), 8.78 (d, J = 8.4 Hz, 1H), 8.43 (dd, J = 8.4, 1.6 Hz, 1H), 8.20 (d, J = 2.0 Hz, 1H), 7.68 (dd, J = 8.8, 2.8 Hz, 1H), 7.41 (t, J = 53.6 Hz, 1H), 6.77 (d, J = 8.8 Hz, 1H), 5.27 (s, 2H), 3.80 (s, 3H). |
| 174 | 2-((6-methoxypyridin-3-yl)methyl)-6-(2- methylthiazol-4-ylsulfonyl)phthalazin-1(2H)-one 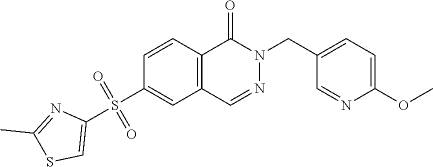 | MS (ES⁺): 429.0 (M + H)⁺ ¹H NMR (400 MHz, DMSO-d6) δ: 8.70 (s, 1H), 8.68 (s, 1H), 8.66(d, J = 0.8 Hz, 1H), 8.48 (d, J= 8.4 Hz, 1H), 8.30 (dd, J1 = 2.0 Hz, J2 = 8.4 Hz, 1H), 8.20(d, J = 2.0 Hz, 1H), 7.69 (dd, J1 = 2.4 Hz, J2 = 8.4 Hz, 1H), 6.77 (d, J = 8.4 Hz, 1H), 5.28 (s, 2H), 3.81 (s, 3H), 2.64 (s, 3H). |
| 175 | 2-((6-methoxypyridin-3-yl)methyl)-6-(6- methoxypyridin-3-ylsulfonyl)phthalazin-1(2H)-one 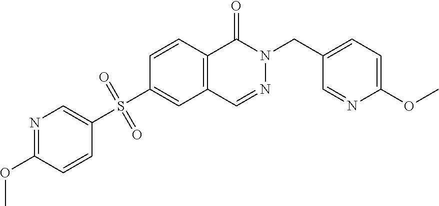 [SMILES: COc1ccc(Cn2ncc3cc(S(=O)(=O)c4ccc(OC)nc4)ccc3c2=O)cn1] | MS (ES⁺): 439.0 (M + H)⁺ ¹H NMR (400 MHz, DMSO-d6) δ: 8.86 (t, J = 2.0 Hz, 1H), 8.69 (d, J = 1.2 Hz, 1H), 8.61 (s, 1H), 8.43 (d, J = 8.4 Hz, 1H), 8.36 (d, J = 2.0 Hz, 1H), 8.23 (dd, J = 8.8, 2.8 Hz, 1H), 8.19 (d, J = 2.0 Hz, 1H), 7.67 (dd, J = 8.4, 2.4 Hz, 1H), 7.04 (dd, J = 8.8, 0.4 Hz, 1H) , 6.76 (d, J = 8.8 Hz, 1H), 5.27 (s, 2H), 3.93 (s, 3H), 3.81 (s, 3H). |
| 176 | 2-((6-methoxypyridin-3-yl)methyl)-6-(2- methylbenzo[d]thiazol-4-ylsulfonyl)phthalazin-1(2H)-one 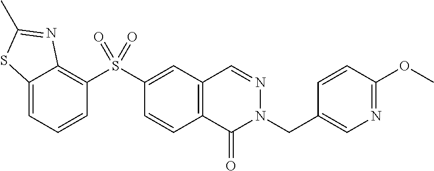 | MS (ES⁺): 478.9 (M + H)⁺ ¹H NMR (400 MHz, DMSO-d6) δ: 8.81 (d, J = 1.2 Hz, 1H), 8.68 (s, 1H), 8.50-8.47 (m, 2H), 8.42 (d, J = 8.4 Hz, 1H), 8.26 (dd, J =7.6, 0.8 Hz, 1H), 8.19 (d, J = 2.0 Hz, 1H), 7.71-7.67 (m, 2H), 6.75 (d, J = 8.8 Hz, 1H), 5.26 (s, 2H), 3.80 (s, 3H), 2.83 (s, 3H). |
| 177 | 6-(5-methoxypyrazin-2-ylsulfonyl)-2-((6- methoxypyridin-3-yl)methyl)phthalazin-1(2H)-one 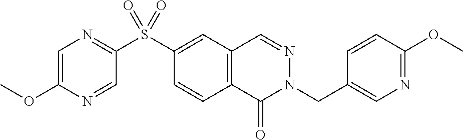 | MS (ES⁺): 439.9 (M + H)⁺ ¹H NMR (400 MHz, DMSO-d6) δ: 9.06 (d, J = 1.2 Hz, 1H), 8.68 (d, J = 1.6 Hz, 1H), 8.65 (s, 1H), 8.46 (d, J = 8.8, 1H), 8.41 (d, J = 1.6 Hz, 1H), 8.33 (dd, J = 8.4, 2.0 Hz, 1H), 8.20 (d, J = 2.0 Hz, 1H), 7.68 (dd, J = 8.4, 2.4 Hz, 1H), 6.77 (d, J = 8.8 Hz, 1H), 5.28 (s, 2H), 4.00 (s, 3H), 3.81 (s, 3H). |
| 178 | 6-(imidazo[1,2-a]pyridin-5-ylsulfonyl)-2- ((6-methoxypyridin-3-yl)methyl)phthalazin-1(2H)-one 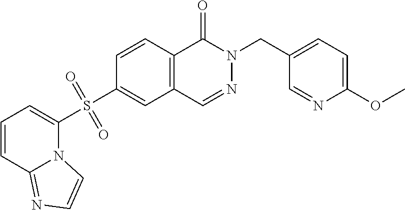 [SMILES: COc1ccc(Cn2ncc3cc(S(=O)(=O)c4cccc5nccn45)ccc3c2=O)cn1] | MS (ES⁺): 448.0 (M + H)⁺ ¹H NMR (400 MHz, DMSO-d6) δ: 8.97 (s, 1H), 8.62 (s, 1H), 8.43 (s, 2H), 8.29 (s, 1H), 8.18 (d, J = 2.4 Hz, 1H), 8.06 (t, J = 7.6 Hz, 2H), 7.78 (s, 1H), 7.67 (dd, J1 = 2.4 Hz, J2 = 8.8 Hz, 1H), 7.56 (t, J = 8.0 Hz, 1H), 5.26 (s, 2H), 3.80 (s, 3H). |
| 179 | 6-(1-(difluoromethyl)-1H-pyrazol-3- ylsulfonyl)-2-((6-methoxypyridin-3-yl)methyl)phthalazin-1(2H)-one 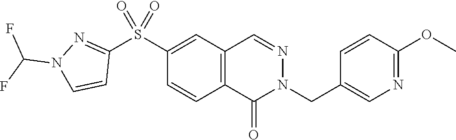 [SMILES: COc1ccc(Cn2ncc3cc(S(=O)(=O)c4ccn(C(F)F)n4)ccc3c2=O)cn1] | MS (ES⁺): 447.9 (M + H)⁺ ¹H NMR (400 MHz, DMSO-d6) δ: 9.16 (s, 1H), 8.68 (d, J = 1.6 Hz, 1H), 8.64 (s, 1H), 8.47 (d, J = 8.4 Hz, 1H), 8.40 (s, 1H), 8.37 (dd, J = 8.4, 2.0 Hz, 1H), 8.20 (d, J = 2.0 Hz, 1H), 7.86 (t, J = 58 Hz, 1H), 7.69 (dd, J = 8.4, 2.4 Hz, 1H), 6.77 (d, J = 8.4 Hz, 1H), 5.29 (s, 2H), 3.81 (s, 3H). |
| 182 | 6-((2,3-dihydrobenzo[b][1,4]dioxin-6- yl)sulfonyl)-2-((6-methoxypyridin-3-yl)methyl)phthalazin-1(2H)-one 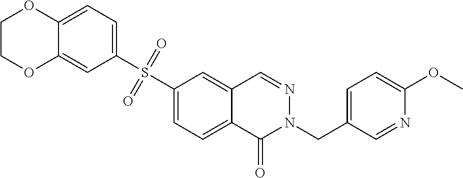 [SMILES: COc1ccc(Cn2ncc3cc(S(=O)(=O)c4ccc5c(c4)OCCO5)ccc3c2=O)cn1] | MS (ES⁺): 466.2 (M + H)⁺ ¹H NMR (400 MHz, DMSO-d6) δ 8.64 (d, J = 1.8 Hz, 1H), 8.60 (s, 1H), 8.41 (d, J = 8.5 Hz, 1H), 8.29 (dd, J = 8.4, 1.9 Hz, 1H), 8.19 (d, J = 2.4 Hz, 1H), 7.67 (dd, J = 8.5, 2.5 Hz, 1H), 7.52-7.43 (m, 2H), 7.13- 7.06 (m, 1H), 6.76 (d, J = 8.5 Hz, 1H), 5.27 (s, 2H), 4.34-4.25 (m, 4H), 3.80 (s, 3H). |
| 183 | 6-((2,3-dihydrofuro[2,3-b]pyridin-5- yl)sulfonyl)-2-((6-methoxypyridin-3-yl)methyl)phthalazin-1(2H)-one 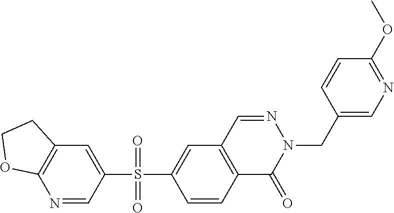 [SMILES: COc1ccc(Cn2ncc3cc(S(=O)(=O)c4cnc5c(c4)CCO5)ccc3c2=O)cn1] | MS (ES⁺): 451.2 (M + H)⁺ ¹H NMR (400 MHz, DMSO-d6) δ 8.67 (d, J = 1.8 Hz, 1H), 8.64-8.57 (m, 2H), 8.43 (d, J = 8.4 Hz, 1H), 8.33 (dd, J = 8.4, 1.9 Hz, 1H), 8.19 (d, J = 2.5 Hz, 1H), 8.16-8.10 (m, 1H), 7.68 (dd, J = 8.5, 2.5 Hz, 1H), 6.76 (d, J = 8.6 Hz, 1H), 5.27 (s, 2H), 4.68 (t, J = 8.7 Hz, 2H), 3.81 (s, 3H), 3.27 (t, J = 8.7 Hz, 2H). |
| 184 | 6-(imidazo[1,2-a]pyridin-7-ylsulfonyl)-2- ((6-methoxypyridin-3-yl)methyl)phthalazin-1(2H)-one 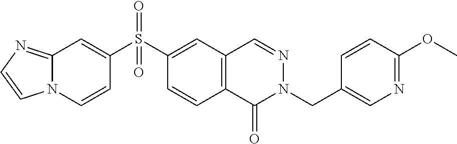 [SMILES: COc1ccc(Cn2ncc3cc(S(=O)(=O)c4ccn5ccnc5c4)ccc3c2=O)cn1] | MS (ES⁺): 448.0 (M + H)⁺ ¹H NMR (400 MHz, DMSO-d6) δ: 8.78-8.75 (m, 2H), 8.61 (s, 1H), 8.45-8.39 (m, 1H), 8.34(d, J = 0.8 Hz, 1H), 8.21-8.18(m, 2H), 7.89 (d, J = 1.2 Hz, 1H), 7.67 (dd, J1 = 2.4 Hz, J2 = 8.4 Hz, 1H), 7.33 (dd, J1 = 2.0 Hz, J2 = 7.2 Hz, 1H), 6.76 (d, J = 8.4 Hz, 1H), 5.27 (s, 2H), 3.80 (s, 3H). |
| 185 | 6-(1,2-dimethyl-1H-imidazol-4-ylsulfonyl)- 2-((6-methoxypyridin-3-yl)methyl)phthalazin-1(2H)-one 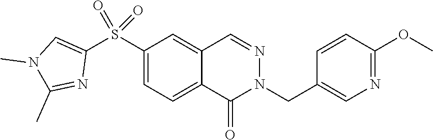 | MS (ES⁺): 425.9 (M + H)⁺ ¹H NMR (400 MHz, DMSO-d6) δ: 8.67 (s, 1H), 8.55 (d, J = 1.6 Hz, 1H), 8.43 (d, J = 8.4 Hz, 1H), 8.23 (d, J = 2.0 Hz, 1H), 8.20 (dd, J = 4.4, 2.0 Hz, 1H), 8.06 (s, 1H), 7.69 (dd, J = 8.4, 2.4 Hz, 1H), 6.77 (d, J = 8.4 Hz, 1H), 5.28 (s, 2H), 3.81 (s, 3H), 3.60 (s, 3H), 2.25 (s, 3H). |
| 190 | 6-(1,5-dimethyl-1H-pyrazol-4-ylsulfonyl)- 2-((6-methoxypyridin-3-yl)methyl)phthalazin-1(2H)-one 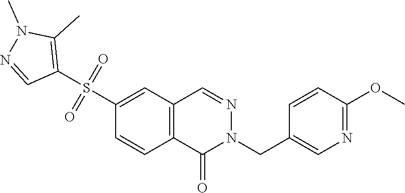 [SMILES: COc1ccc(Cn2ncc3cc(S(=O)(=O)c4cnn(C)c4C)ccc3c2=O)cn1] | MS (ES⁺): 426.0 (M + H)⁺ ¹H NMR (400 MHz, DMSO-d6) δ: 8.64 (s, 1H), 8.59 (d, J = 1.6 Hz, 1H), 8.42 (d, J = 8.4 Hz 1H), 8.26 (dd, J = 8.4, 2.0 Hz, 1H), 8.20 (d, J = 2.4 Hz, 1H), 7.94 (s, 1H), 7.69 (dd, J = 8.8, 2.4 Hz, 1H), 6.77 (t, J = 8.4 Hz, 1H), 5.28 (s, 2H), 3.81 (s, 3H), 3.74 (s, 3H), 2.43 (s, 3H). |
| 191 | (R)-6-(4-methoxyphenylsulfonimidoyl)-2- ((6-methoxypyridin-3-yl)methyl)phthalazin-1(2H)-one 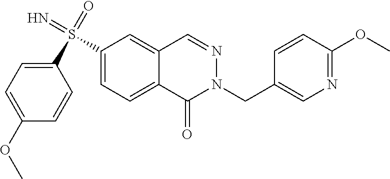 [SMILES: COc1ccc([S@@](=N)(=O)c2ccc3c(=O)n(Cc4ccc(OC)nc4)ncc3c2)cc1] | MS (ES⁺): 437 (M + H)⁺ ¹H NMR (400 MHz, DMSO-d6) δ 8.61 (d, J = 0.7 Hz, 1H), 8.57 (d, J = 1.8 Hz, 1H), 8.36 (d, J = 8.4 Hz, 1H), 8.26 (dd, J = 8.4, 1.8 Hz, 1H), 8.18 (d, J = 2.4 Hz, 1H), 7.96- 7.87 (m, 2H), 7.66 (dd, J = 8.5, 2.5 Hz, 1H), 7.13-7.05 (m, 2H), 6.75 (dd, J = 8.5, 0.7 Hz, 1H), 5.26 (s, 2H), 5.23 (s, 1H), 3.80 (s, 3H), 3.80 (s, 3H). RT: 4.88 min, 100% ee |
| 192 | (S)-6-(4-methoxyphenylsulfonimidoyl)-2- ((6-methoxypyridin-3-yl)methyl)phthalazin-1(2H)-one 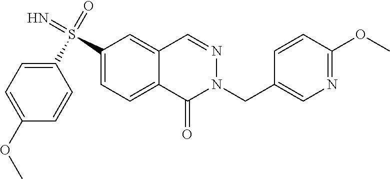 [SMILES: COc1ccc([S@](=N)(=O)c2ccc3c(=O)n(Cc4ccc(OC)nc4)ncc3c2)cc1] | MS (ES⁺): 437 (M + H)⁺ ¹H NMR (400 MHz, DMSO-d6) δ 8.61 (d, J = 0.7 Hz, 1H), 8.57 (d, J = 1.8 Hz, 1H), 8.36 (d, J = 8.4 Hz, 1H), 8.26 (dd, J = 8.4, 1.8 Hz, 1H), 8.18 (d, J = 2.4 Hz, 1H), 7.96- 7.87 (m, 2H), 7.66 (dd, J = 8.5, 2.5 Hz, 1H), 7.13-7.05 (m, 2H), 6.75 (dd, J = 8.5, 0.7 Hz, 1H), 5.26 (s, 2H), 5.23 (s, 1H), 3.80 (s, 3H), 3.80 (s, 3H). RT: 8.80 min, 100% ee |

### Example 193—6-(1-cyclopropyl-1H-pyrazol-4-ylsulfonyl)-2-((2-hydroxypyridin-3-yl)methyl)phthalazin-1(2H)-one

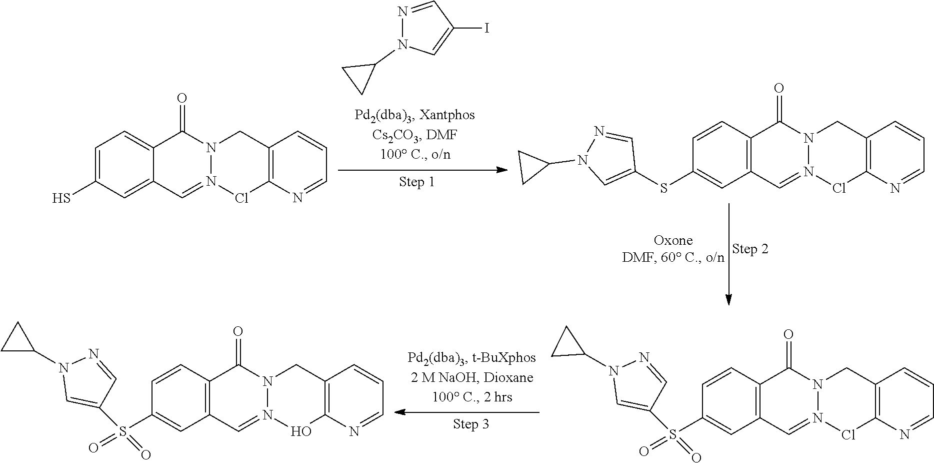

### Step 1

To a mixture of 2-((2-chloropyridin-3-yl)methyl)-6-mercaptophthalazin-1(2H)-one (Intermediate 46, 300 mg, 1 mmol), Pd₂(dba)₃ (91.5 mg, 0.1 mmol), Xantphos (115.6 mg, 0.2 mmol), Cs₂CO₃ (652 mg, 2 mmol) in DMF (5 mL) was added 1-cyclopropyl-4-iodo-1H-pyrazole (281 mg, 1.2 mmol) at room temperature, and the reaction mixture was stirred at 100° C. overnight under N₂. After LCMS indicated the reaction was complete, the reaction mixture was filtered through Celite and washed with EtOAc. The filtrate was diluted with water (10 mL) and extracted with EtOAc (10 mL×3). The combined organic layer was washed by brine, dried over Na₂SO₄, filtered and concentrated at 45° C. under reduced pressure. The residue was purified by flash column chromatography to give 2-((2-chloropyridin-3-yl)methyl)-6-(1-cyclopropyl-1H-pyrazol-4-ylthio)phthalazin-1(2H)-one (310 mg) as a yellow solid. MS (ES⁺): 410.0 (M+H)⁺

### Step 2

To a solution of 2-((2-chloropyridin-3-yl)methyl)-6-(1-cyclopropyl-1H-pyrazol-4-ylthio)phthalazin-1(2H)-one (310 mg, 0.76 mmol) in DMF (3 mL) was added OXONE® (1.5 g, 2.4 mmol) at RT, and the reaction mixture was stirred at 60° C. overnight. After LCMS indicated the reaction was complete, the reaction mixture was filtered through Celite and washed with EtOAc. The filtrate was diluted with water (10 mL) and extracted with EtOAc (5 mL×3). The combined organic layer was washed by brine, dried over Na₂SO₄, filtered and concentrated at 45° C. under reduced pressure. The residue was purified by flash column chromatography to give 2-((2-chloropyridin-3-yl)methyl)-6-(1-cyclopropyl-1H-pyrazol-4-ylsulfonyl)phthalazin-1(2H)-one (260 mg) as white solid. MS (ES⁺): 441.9 (M+H)⁺

### Step 3

To a mixture of 2-((2-chloropyridin-3-yl)methyl)-6-(1-cyclopropyl-1H-pyrazol-4-ylsulfonyl)phthalazin-1(2H)-one (260 mg, 0.6 mmol), Pd₂(dba)₃ (55 mg, 0.06 mmol), t-BuXphos (51 mg, 0.12 mmol) in dioxane (10 mL) was added NaOH (240 mg, 6 mmol) at RT, and the reaction mixture was stirred at 100° C. for 2 h under N₂. After LCMS indicated the reaction was complete, the reaction mixture was filtered through Celite and washed with EtOAc. The filtrate was diluted with water (10 mL) and extracted with EtOAc (10 mL×3). The combined organic layer was washed by brine, dried over Na₂SO₄, filtered and concentrated at 45° C. under reduced pressure and the mixture was purified by reversed column chromatography. The reversed column chromatography fraction was concentrated at 30° C. under reduced pressure to remove MeCN, and the residue was lyophilized to afford the title compound (19.56 mg) as white solid. ¹H NMR (400 MHz, DMSO-d6) δ: 11.72 (br, 1H), 8.69 (s, 1H), 8.62 (s, 1H), 8.62 (d, J=1.6 Hz, 1H), 8.44 (d, J=8.4 Hz 1H), 8.32 (dd, J=8.4, 2.0 Hz, 1H), 8.02 (s, 1H), 7.31 (d, J=4.8 Hz, 1H), 7.20 (dd, J=6.4, 1.6 Hz, 1H), 6.08 (t, J=6.8 Hz, 1H), 5.09 (s, 2H), 3.85-3.82 (m, 1H), 1.10-1.08 (m, 2H), 1.02-0.97 (m, 2H). MS (ES⁺): 424.0 (M+H)⁺

### Example 194—6-(1-cyclopropyl-1H-pyrazol-4-ylsulfonyl)-2-((1-methyl-1H-pyrazol-3-yl)methyl)phthalazin-1(2H)-one

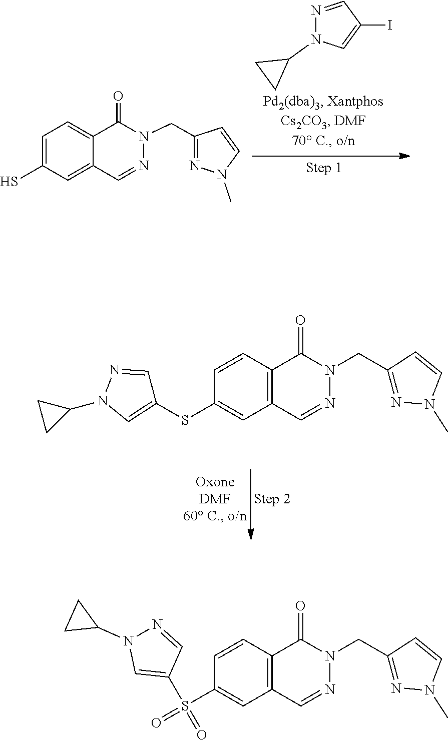 [SMILES: Ic1cnn(C2CC2)c1 ; Cn1ccc(Cn2ncc3cc(S)ccc3c2=O)n1 ; Cn1ccc(Cn2ncc3cc(Sc4cnn(C5CC5)c4)ccc3c2=O)n1 ; Cn1ccc(Cn2ncc3cc(S(=O)(=O)c4cnn(C5CC5)c4)ccc3c2=O)n1]

### Step 1

To a mixture of 6-mercapto-2-((1-methyl-1H-pyrazol-3-yl)methyl)phthalazin-1(2H)-one (Intermediate 48, 400 mg, 1.5 mmol), Pd₂(dba)3 (137 mg, 0.15 mmol), Xantphos (173 mg, 0.3 mmol), Cs₂CO₃ (978 mg, 3 mmol) in DMF (5 mL) was added 1-cyclopropyl-4-iodo-1H-pyrazole (398 mg, 1.7 mmol) at RT, and the reaction mixture was stirred at 70° C. for 4 h under N₂. After LCMS indicated the reaction is completed, the reaction mixture was filtered through Celite and washed with EtOAc. The filtrate was diluted with water (10 mL) and extracted with EtOAc (10 mL×3). The combined organic layer was washed by brine, dried over Na₂SO₄, filtered and concentrated at 45° C. under reduced pressure. The residue was purified by flash column chromatography to give 6-(1-cyclopropyl-1H-pyrazol-4-ylthio)-2-((1-methyl-1H-pyrazol-3-yl)methyl)phthalazin-1(2H)-one (200 mg) as a yellow solid. MS (ES⁺): 379.0 (M+H)⁺

### Step 2

To a solution of 6-(1-cyclopropyl-1H-pyrazol-4-ylthio)-2-((1-methyl-1H-pyrazol-3-yl)methyl)phthalazin-1(2H)-one (200 mg, 0.53 mmol) in DMF (4 mL) was added OXONE® (1.1 g, 1.8 mmol) at RT, and the reaction mixture was stirred at 60° C. overnight. After LCMS indicated the reaction completed, the reaction mixture was filtered and washed by DCM (10 mL). The filtrate was diluted with water (20 mL) and extracted with DCM (10 mL×3). The organic layer was concentrated at 30° C. under reduced pressure and the mixture was purified by reversed column chromatography. The reversed column chromatography fractions were concentrated at 30° C. under reduced pressure to remove MeCN, and the residue was lyophilized to give the title compound (99.38 mg) as a white solid. ¹H NMR (400 MHz, DMSO-d6) δ: 8.68 (s, 1H), 8.59 (s, 2H), 8.44 (d, J=8.4 Hz, 1H), 8.30 (dd, J=8.4, 1.6 Hz, 1H), 8.01 (s, 1H), 7.57 (d, J=2.4 Hz, 1H), 6.10 (d, J=2.4 Hz, 1H), 5.26 (s, 2H), 3.86-3.80 (m, 1H), 3.76 (s, 3H), 1.12-1.05 (m, 2H), 1.01-0.96 (m, 2H). MS (ES⁺): 411.0 (M+H)⁺

### Example 195—2-((1H-pyrazol-3-yl)methyl)-6-(benzofuran-5-ylsulfonyl)phthalazin-1(2H)-one

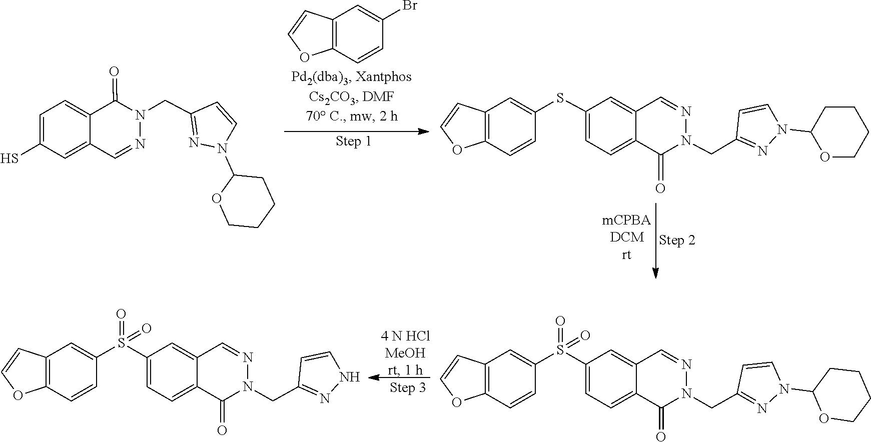 [SMILES: Brc1ccc2occc2c1 ; O=c1c2ccc(S)cc2cnn1Cc1ccn(C2CCCCO2)n1 ; O=c1c2ccc(Sc3ccc4occc4c3)cc2cnn1Cc1ccn(C2CCCCO2)n1 ; O=c1c2ccc(S(=O)(=O)c3ccc4occc4c3)cc2cnn1Cc1cc[nH]n1 ; O=c1c2ccc(S(=O)(=O)c3ccc4occc4c3)cc2cnn1Cc1ccn(C2CCCCO2)n1]

### Step 1

A mixture of 6-mercapto-2-((1-(tetrahydro-2H-pyran-2-yl)-1H-pyrazol-3-yl)methyl)phthalazin-1(2H)-one (Intermediate 49, 50 mg, 1 Eq, 0.15 mmol), 5-bromobenzofuran (43 mg, 1.5 Eq, 0.22 mmol), cesium carbonate (95 mg, 2 Eq, 0.29 mmol), Pd₂dba₃ (6.7 mg, 0.05 Eq, 7.3 μmol) and Xantphos (8.4 mg, 0.1 Eq, 15 μmol) in DMF (1.5 mL) was stirred at 120° C. under microwave radiation for 2 h, then allowed to cool to RT. Water and DCM were added and the layers separated through a phase separator. The organic layer was washed with brine, then absorbed on silica.

The crude product was purified by chromatography on silica gel to afford 6-(benzofuran-5-ylthio)-2-((1-(tetrahydro-2H-pyran-2-yl)-1H-pyrazol-3-yl)methyl)phthalazin-1(2H)-one (28 mg) as a pale yellow oil/glass. MS (ES⁺): 459 (M+H)⁺

### Step 2

mCPBA (35 mg, 70% Wt, 2.5 Eq, 0.14 mmol) was added to a solution of 6-(benzofuran-5-ylthio)-2-((1-(tetrahydro-2H-pyran-2-yl)-1H-pyrazol-3-yl)methyl)phthalazin-1(2H)-one (28 mg, 92% Wt, 1 Eq, 56 μmol) in DCM (1.5 mL) and the mixture was stirred for 1 h. 2 N NaOH and DCM were added and the layers separated through a phase separator. The organic layer was washed with brine, dried (MgSO₄) and concentrated under vacuum to afford crude 6-(benzofuran-5-ylsulfonyl)-2-((1-(tetrahydro-2H-pyran-2-yl)-1H-pyrazol-3-yl)methyl)phthalazin-1(2H)-one (30 mg) as a pale yellow solid. MS (ES⁺): 491 (M+H)⁺

### Step 3

HCl (4 N in dioxane) (0.23 mL, 4 molar, 15 Eq, 0.92 mmol) was added to a solution of 6-(benzofuran-5-ylsulfonyl)-2-((1-(tetrahydro-2H-pyran-2-yl)-1H-pyrazol-3-yl)methyl)phthalazin-1(2H)-one (30 mg, 1 Eq, 61 μmol) in MeOH (0.5 mL) and the mixture was stirred for 1 h, then concentrated under vacuum. Sat. NaHCO₃ and DCM were added and the layers separated through a phase separator. The organic layer was washed with brine, then absorbed on silica. The crude product was purified by chromatography on silica gel to afford the title compound (19.8 mg) as a white solid. ¹H NMR (DMSO-d6) δ: 12.64 (s, 1H), 8.71-8.66 (m, 1H), 8.59 (s, 1H), 8.46-8.39 (m, 2H), 8.32 (dd, J=8.4, 1.9 Hz, 1H), 8.22 (d, J=2.3 Hz, 1H), 7.95 (dd, J=8.8, 2.0 Hz, 1H), 7.88 (d, J=8.7 Hz, 1H), 7.63-7.58 (m, 1H), 7.16 (dd, J=2.3, 0.9 Hz, 1H), 6.11 (d, J=2.1 Hz, 1H), 5.29 (s, 2H). MS (ES⁺): 407 (M+H)⁺

### Example 196—2-((1-cyclopropyl-1H-pyrazol-3-yl)methyl)-6-((1-cyclopropyl-1H-pyrazol-4-yl)sulfonyl)phthalazin-1(2H)-one

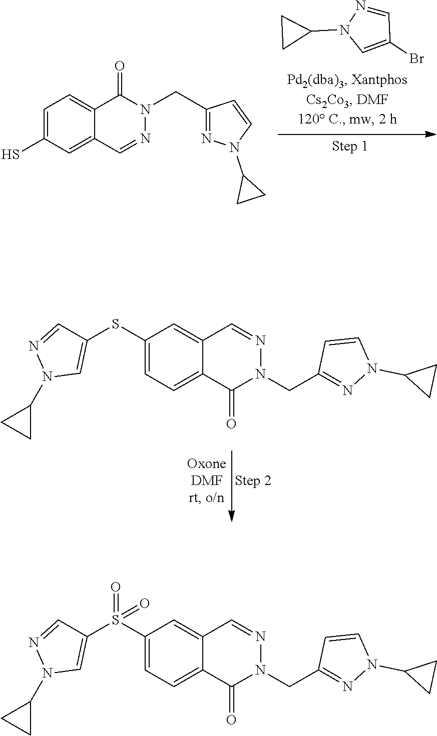 [SMILES: Brc1cnn(C2CC2)c1 ; O=c1c2ccc(S)cc2cnn1Cc1ccn(C2CC2)n1 ; O=c1c2ccc(Sc3cnn(C4CC4)c3)cc2cnn1Cc1ccn(C2CC2)n1 ; O=c1c2ccc(S(=O)(=O)c3cnn(C4CC4)c3)cc2cnn1Cc1ccn(C2CC2)n1]

### Step 1

DMF (1 mL) was sparged with nitrogen for 5 minutes. 2-((1-cyclopropyl-1H-pyrazol-3-yl)methyl)-6-mercaptophthalazin-1(2H)-one (Intermediate 50, 50 mg, 86% Wt, 1 Eq, 0.14 mmol), 4-bromo-1-cyclopropyl-1H-pyrazole (38 mg, 20 μL, 1.4 Eq, 0.20 mmol), cesium carbonate (110 mg, 2.3 Eq, 338 μmol), Xantphos (10 mg, 0.12 Eq, 17 μmol) and Pd₂(dba)₃ (8 mg, 0.06 Eq, 9 μmol) were added sequentially. The mixture was heated at 120° C. for 90 minutes in the microwave and then allowed to cool to RT. Additional 4-bromo-1-cyclopropyl-1H-pyrazole (38 mg, 20 μL, 1.4 Eq, 0.20 mmol), Xantphos (10 mg, 0.12 Eq, 17 μmol) and Pd₂(dba)₃ (8 mg, 0.06 Eq, 9 μmol) were added and the reaction mixture was heated at 120° C. for 60 minutes in the microwave and then allowed to cool to RT. The reaction mixture was concentrated in vacuo and the residue was azeotroped with toluene (2 times) to afford the crude product. The crude product was purified by chromatography on silica gel to afford 2-((1-cyclopropyl-1H-pyrazol-3-yl)methyl)-6-((1-cyclopropyl-1H-pyrazol-4-yl)thio)phthalazin-1(2H)-one (9.0 mg) as a brown oil. MS (ES⁺): 405 (M+H)⁺

### Step 2

A suspension of 2-((1-cyclopropyl-1H-pyrazol-3-yl)methyl)-6-((1-cyclopropyl-1H-pyrazol-4-yl)thio)phthalazin-1(2H)-one (9.0 mg, 80% Wt, 1 Eq, 18 μmol) and OXONE® (35 mg, 3.2 Eq, 57 μmol) in DMF (1 mL) was stirred at RT for 21 h. The reaction mixture was diluted with DCM (5 mL) and washed with water (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford impure product. The crude product was purified by chromatography on RP Flash C18 to afford the title compound (5.0 mg) as a pale yellow solid. ¹H NMR (400 MHz, DMSO-d6) δ 8.67 (s, 1H), 8.59 (d, J=2.1 Hz, 2H), 8.43 (d, J=8.4 Hz, 1H), 8.30 (dd, J=8.5, 1.9 Hz, 1H), 8.00 (d, J=0.8 Hz, 1H), 7.65 (d, J=2.3 Hz, 1H), 6.07 (d, J=2.3 Hz, 1H), 5.24 (s, 2H), 3.83 (tt, J=7.5, 3.9 Hz, 1H), 3.62 (tt, J=7.4, 3.6 Hz, 1H), 1.13-1.05 (m, 2H), 1.01-0.94 (m, 4H), 0.93-0.87 (m, 2H). MS (ES⁺): 437 (M+H)⁺

### Example 197—6-((1H-pyrazol-4-yl)sulfonyl)-2-((6-methoxypyridin-3-yl)methyl)phthalazin-1(2H)-one

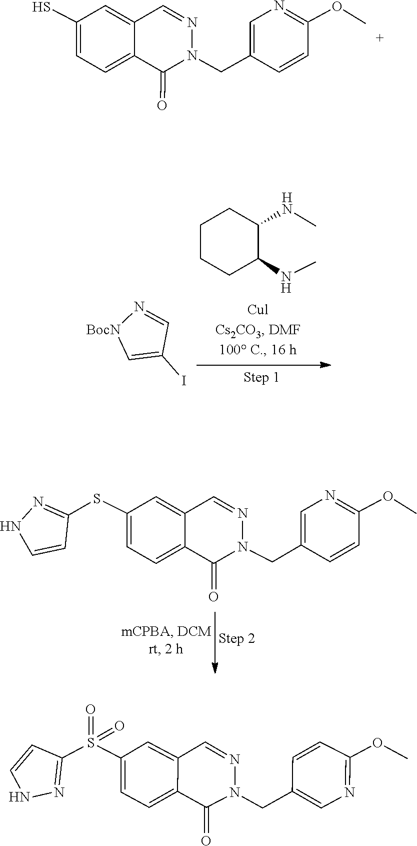

### Step 1

An oven-dried vial was cooled to RT under N₂, then charged with copper(I) iodide (44 mg, 0.23 mmol), cesium carbonate (348 mg, 1.07 mmol), and tert-butyl 4-iodo-1H-pyrazole-1-carboxylate (118 mg, 1.2 Eq, 401 μmol). Dry DMF (6 ml) was added, followed by 1-N,2-N-dimethylcyclohexane-1,2-diamine (95.0 mg, 86.3 μL, 2 Eq, 668 μmol) and 6-mercapto-2-((6-methoxypyridin-3-yl)methyl)phthalazin-1(2H)-one (Intermediate 40, 100 mg, 1 Eq, 334 μmol). The resulting blue suspension was stirred at RT for 5 minutes, then heated to 100° C. for 16 h. The reaction mixture was then cooled to RT, diluted with ethyl acetate (25 ml), then transferred to a separatory funnel, and washed with saturated aqueous ammonium chloride (30 mL). The aqueous phase was extracted with ethyl acetate (3×30 ml). The combined organic phases were washed with water (3×25 ml), brine (30 mL), dried over magnesium sulfate and evaporated under reduced pressure. The crude product was purified by chromatography on silica gel to afford 6-((1H-pyrazol-4-yl)thio)-2-((6-methoxypyridin-3-yl)methyl)phthalazin-1(2H)-one (40 mg) as a sticky orange solid. MS (ES)⁺: 366 (M+H)⁺

### Step 2

mCPBA (59 mg, 5 Eq, 0.34 mmol) was added to a stirred solution of 6-((1H-pyrazol-4-yl)thio)-2-((6-methoxypyridin-3-yl)methyl)phthalazin-1(2H)-one (40 mg, 62% Wt, 1 Eq, 68 μmol) in DCM (2 mL) at RT and the mixture was stirred for 2 h. The reaction mixture was diluted with DCM (10 mL) and washed with saturated NaHCO₃ (10 mL) and water (10 mL). The organic phase was collected, dried (phase separator) and evaporated under reduced pressure. The crude product was purified by chromatography on silica gel to afford the title compound (8 mg) as a clear white solid. ¹H NMR (400 MHz, DMSO-d6) δ 8.59 (s, 1H), 8.38 (d, J=1.7 Hz, 1H), 8.32 (d, J=8.4 Hz, 1H), 8.18 (d, J=2.4 Hz, 1H), 8.13 (dd, J=8.4, 1.8 Hz, 1H), 7.66 (d, J=7.1 Hz, 3H), 6.75 (d, J=8.5 Hz, 1H), 5.25 (s, 2H), 3.81 (s, 3H) (exchangeable NH not observed). MS (ES)⁺: 398 (M+H)⁺

### Example 198—2-(3-(difluoromethoxy)benzyl)-6-((1-methyl-1H-pyrazol-3-yl)sulfonyl)phthalazin-1(2H)-one

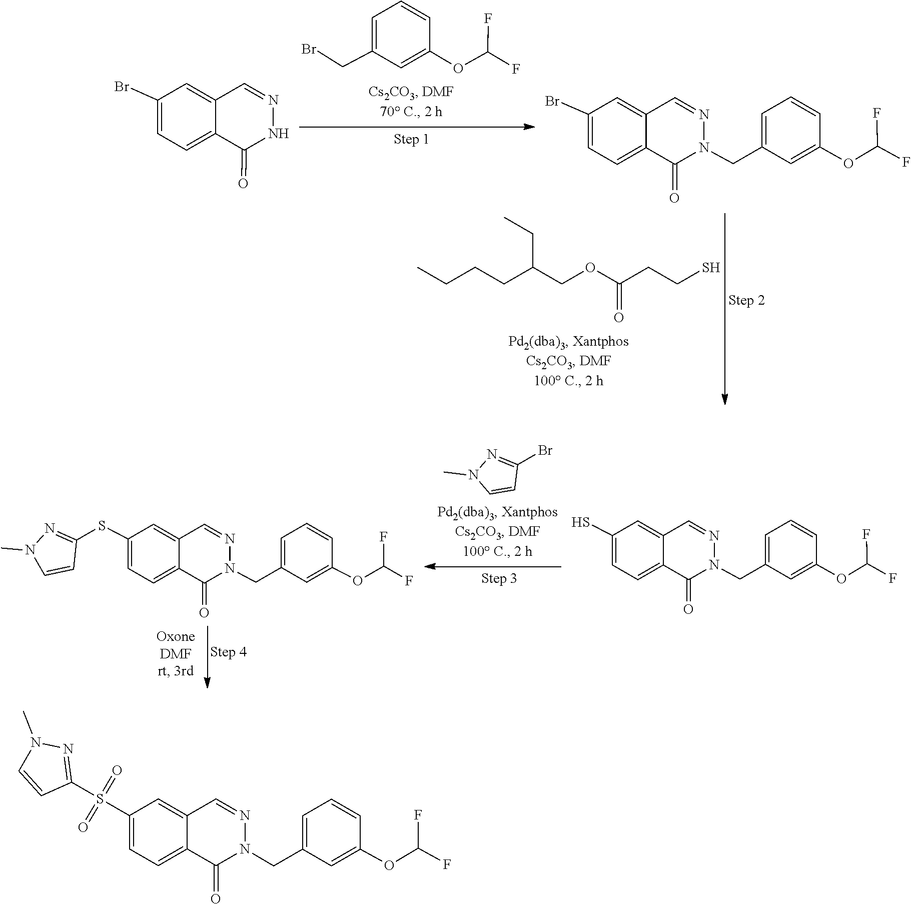

### Step 1

Cesium carbonate (543 mg, 1.5 Eq, 1.67 mmol) was added to a stirred solution of 6-bromophthalazin-1(2H)-one (0.250 g, 1 Eq, 1.11 mmol) in dry DMF (8 mL). The reaction mixture was stirred for 5 minutes, then 1-(bromomethyl)-3-(difluoromethoxy)benzene (290 mg, 1.1 Eq, 1.22 mmol) was added. The resulting mixture was stirred at 70° C. for 2 h. After cooling to RT, the reaction mixture was diluted with water (20 mL) and the resulting precipitate was filtered, washing with water and MTBE, giving 6-bromo-2-(3-(difluoromethoxy)benzyl)phthalazin-1(2H)-one (0.41 g) as a clear white solid. MS (ES⁺): 381/383 (M+H)⁺

### Step 2

A mixture of 6-bromo-2-(3-(difluoromethoxy)benzyl)phthalazin-1(2H)-one (0.41 g, 1 Eq, 1.1 mmol), 2-ethylhexyl 3-mercaptopropanoate (0.25 g, 0.26 mL, 1.05 Eq, 1.1 mmol), cesium carbonate (0.70 g, 2 Eq, 2.2 mmol), Xantphos (62 mg, 0.1 Eq, 0.11 mmol), and Pd₂(dba)₃ (49 mg, 0.05 Eq, 54 μmol) in DMF (12 mL) was heated to 100° C. for 2 h. After cooling to RT, the reaction mixture was diluted with water (50 mL) and acidified with 1 M HCl, then extracted with EtOAc (2×50 mL). The organic phase was collected, dried over MgSO₄ and evaporated under reduced pressure. The crude product was purified by chromatography on silica gel to afford 2-(3-(difluoromethoxy)benzyl)-6-mercaptophthalazin-1(2H)-one (0.12 g) as a sticky yellow solid. MS (ES⁺): 335 (M+H)⁺

### Step 3

A mixture of 2-(3-(difluoromethoxy)benzyl)-6-mercaptophthalazin-1(2H)-one (120 mg, 70% Wt, 1 Eq, 251 μmol), Pd₂(dba)₃ (11.5 mg, 0.05 Eq, 12.6 μmol), Xantphos (14.5 mg, 0.1 Eq, 25.1 μmol), 3-bromo-1-methyl-1H-pyrazole (60.7 mg, 38.3 μL, 1.5 Eq, 377 μmol), and Cs₂CO₃ (164 mg, 2 Eq, 502 μmol) in DMF (5 mL) was heated to 120° C. under microwave irradiation for 2 h. After cooling to RT, the reaction mixture was absorbed on silica gel and purified by chromatography on silica gel to afford 2-(3-(difluoromethoxy)benzyl)-6-((1-methyl-1H-pyrazol-3-yl)thio)phthalazin-1(2H)-one (60 mg) as a sticky orange oil. MS (ES⁺): 415 (M+H)⁺

### Step 4

OXONE® (0.27 g, 3 Eq, 0.43 mmol) was added to a solution of 2-(3-(difluoromethoxy)benzyl)-6-((1-methyl-1H-pyrazol-3-yl)thio)phthalazin-1(2H)-one (60 mg, 1 Eq, 0.14 mmol) in DMF (6 mL) and the reaction mixture was left to stir for 3 days at RT. The reaction was diluted with water (100 mL) and resulting precipitate was filtered, washing with water and MTBE, giving the crude product as a pale orange solid. The crude product was purified by chromatography on silica gel to afford the title compound (35 mg) as a clear white solid. ¹H NMR (DMSO-d6) δ: 8.68 (s, 1H), 8.64 (d, J=1.8 Hz, 1H), 8.46 (d, J=8.4 Hz, 1H), 8.29 (dd, J=8.5, 1.9 Hz, 1H), 7.97 (d, J=2.4 Hz, 1H), 7.37 (dd, J=7.9 Hz, 1H), 7.20 (t, J=74.1 Hz, 1H), 7.14-7.11 (m, 1H), 7.08 (dd, J=8.1, 2.5 Hz, 1H), 6.95 (d, J=2.4 Hz, 1H), 5.35 (s, 2H), 3.90 (s, 3H). MS (ES⁺): 447 (M+H)⁺

### Example 199—2-(2-fluoro-5-methoxybenzyl)-6-((1-methyl-1H-pyrazol-3-yl)sulfonyl)phthalazin-1(2H)-one

### Step 1

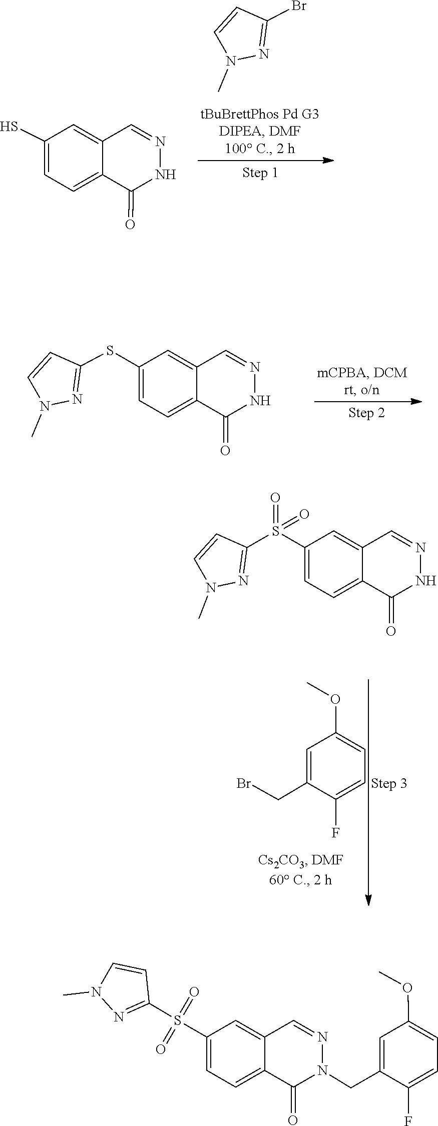

A mixture of 6-mercaptophthalazin-1(2H)-one (Intermediate 53, 300 mg, 50% Wt, 1 Eq, 842 μmol), tBuBrettPhos Pd G3 (36.0 mg, 0.05 Eq, 42.1 μmol), 3-bromo-1-methyl-1H-pyrazole (203 mg, 1.5 Eq, 1.26 mmol), and DIPEA (217 mg, 0.293 mL, 2.00 Eq, 1.68 mmol) in DMF (6 mL) was heated to 100° C. for 2 h. After cooling to RT, the reaction mixture was diluted with DCM and absorbed on silica gel. The crude product was purified by chromatography on silica gel to afford 6-((1-methyl-1H-pyrazol-3-yl)thio)phthalazin-1(2H)-one (100 mg, 0.23 mmol, 28%) as a pale brown solid. MS (ES⁺): 259 (M+H)⁺

### Step 2

mCPBA (120 mg, 3 Eq, 697 μmol) was added to a solution of 6-((1-methyl-1H-pyrazol-3-yl)thio)phthalazin-1(2H)-one (100 mg, 60% Wt, 1 Eq, 232 μmol) in DCM (3.0 mL) and the reaction mixture was left to stir overnight at RT. The reaction was diluted with water (10 mL) and extracted with DCM (2×10 mL). The organic phase was collected, dried (phase separator) and evaporated under reduced pressure. The crude product was purified by chromatography on silica gel to afford 6-((1-methyl-1H-pyrazol-3-yl)sulfonyl)phthalazin-1(2H)-one (30 mg) as a light yellow solid. MS (ES⁺): 291 (M+H)⁺

### Step 3

Cesium Carbonate (70 mg, 2.5 Eq, 0.22 mmol) was added to a stirred solution of 6-((1-methyl-1H-pyrazol-3-yl)sulfonyl)phthalazin-1(2H)-one (25 mg, 1 Eq, 86 μmol) in dry DMF (3 mL). The reaction mixture was stirred for 5 minutes, then 2-(bromomethyl)-1-fluoro-4-methoxybenzene (23 mg, 1.2 Eq, 0.10 mmol) was added. The resulting mixture was stirred at 60° C. for 2 h. After cooling to RT, the reaction mixture was diluted with EtOAc (10 mL) and washed with water (10 mL), and brine (10 mL). The organic phase was collected, dried (MgSO₄) and concentrated under reduced pressure. The crude product was purified by chromatography on silica gel to afford the title compound (36 mg) as a clear white solid. ¹H NMR (400 MHz, DMSO-d6) δ 8.66 (s, 1H), 8.63 (d, J=1.9 Hz, 1H), 8.46 (d, J=8.5 Hz, 1H), 8.29 (dt, J=8.5, 1.8 Hz, 1H), 7.97 (d, J=2.1 Hz, 1H), 7.13 (t, J=9.4 Hz, 1H), 6.95 (d, J=2.1 Hz, 1H), 6.91-6.85 (m, 1H), 6.80 (t, J=4.7 Hz, 1H), 5.34 (s, 2H), 3.91 (s, 3H), 3.67 (s, 3H). MS (ES⁺): 429 (M+H)⁺

### Example 200—2-((2-hydroxypyridin-3-yl)methyl)-6-((1-methyl-1H-pyrazol-4-yl)sulfonyl)phthalazin-1(2H)-one

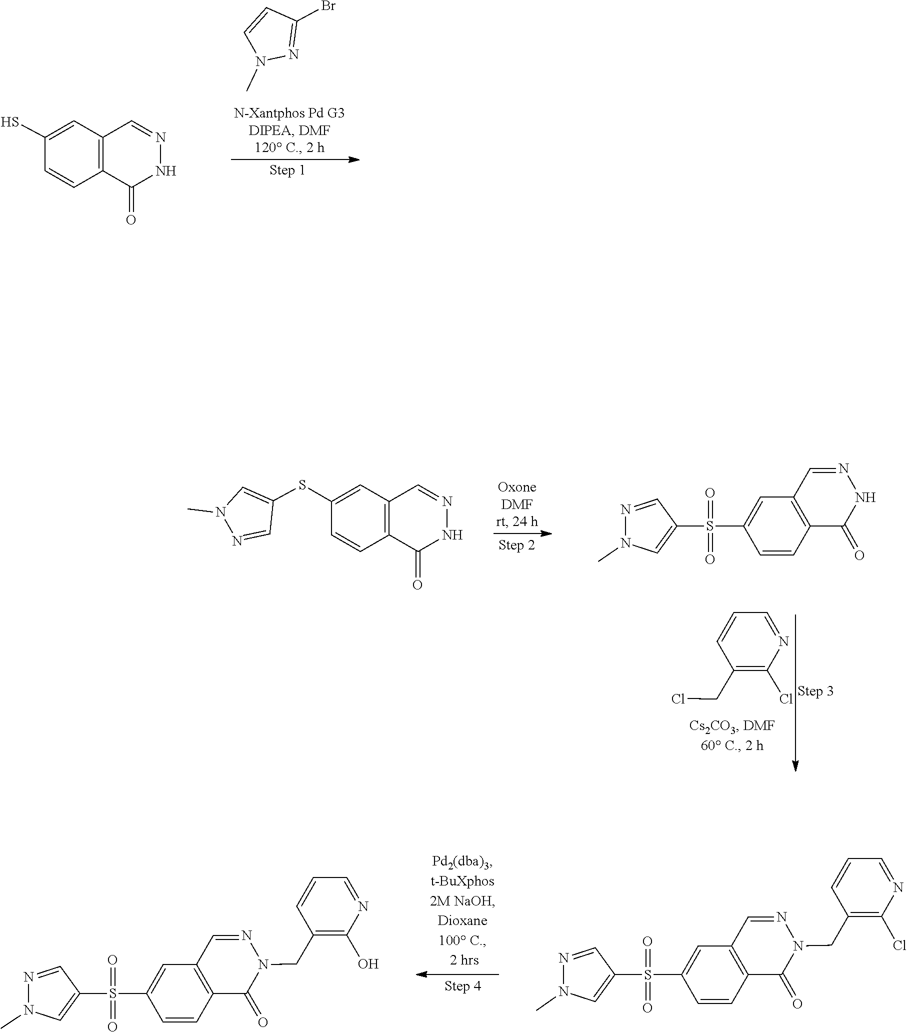

### Step 1

A mixture of 6-mercaptophthalazin-1(2H)-one (Intermediate 53, 0.11 g, 1 Eq, 0.62 mmol), N-Xantphos Pd G3 (28 mg, 0.05 Eq, 31 μmol), 4-bromo-1-methyl-1H-pyrazole (0.13 g, 83 μL, 1.3 Eq, 0.80 mmol), and DIPEA (0.16 g, 0.22 mL, 2 Eq, 1.2 mmol) in DMF (5 mL) was heated to 120° C. under microwave irradiation for 4 h. After cooling to RT, the reaction was diluted with water (20 mL) and acidified with 1 M HCl (20 mL). The mixture was extracted with EtOAc (2×20 mL) and the organic phase was washed with brine (30 mL). The organic layer was collected, dried (MgSO₄) and evaporated under reduced pressure. The crude product was purified by chromatography on silica gel to afford 6-((1-methyl-1H-pyrazol-4-yl)thio)phthalazin-1(2H)-one (50 mg) as a dark orange oil. MS (ES⁺): 259 (M+H)⁺

### Step 2

OXONE® (0.25 g, 3 Eq, 0.40 mmol) was added to a solution of 6-((1-methyl-1H-pyrazol-4-yl)thio)phthalazin-1(2H)-one (50 mg, 69% Wt, 1 Eq, 0.13 mmol) in DMF (6 mL) and the reaction mixture was left to stir for 20 h at RT. The reaction was diluted with water (100 mL) and the resulting precipitate was filtered, washing with water and MTBE, giving crude product as a pale orange solid. The crude product was purified by chromatography on silica gel to afford 6-((1-methyl-1H-pyrazol-4-yl)sulfonyl)phthalazin-1(2H)-one (23 mg) as a pale orange solid. MS (ES⁺): 291 (M+H)⁺

### Step 3

Cesium carbonate (44 mg, 2.0 Eq, 0.13 mmol) was added to a stirred solution of 6-((1-methyl-1H-pyrazol-4-yl)sulfonyl)phthalazin-1(2H)-one (23 mg, 85% Wt, 1 Eq, 67 μmol) in dry DMF (6 mL). The reaction mixture was stirred for 5 minutes, then 2-chloro-3-(chloromethyl)pyridine (Intermediate 45, 12 mg, 1.1 Eq, 74 μmol) was added. The resulting mixture was stirred at 70° C. for 3 h. After cooling to RT, the reaction mixture was diluted with water (20 mL) and extracted with EtOAc (2×20 mL). The organic phase was collected, dried (MgSO₄) and evaporated under reduced pressure, giving crude 2-((2-chloropyridin-3-yl)methyl)-6-((1-methyl-1H-pyrazol-4-yl)sulfonyl)phthalazin-1(2H)-one (20 mg) as a yellow solid. MS (ES⁺): 416/418 (M+H)⁺

### Step 4

tBuXPhos (2.79 mg, 0.2 Eq, 6.58 μmol) and Pd2dba3 (3.01 mg, 0.1 Eq, 3.29 μmol) were added to a stirred solution of 2-((2-chloropyridin-3-yl)methyl)-6-((1-methyl-1H-pyrazol-4-yl)sulfonyl)phthalazin-1(2H)-one (18.0 mg, 76% Wt, 1 Eq, 32.9 μmol) in dry 1,4-dioxane (2 mL) under N₂. 2 N sodium hydroxide (13.2 mg, 164 μL, 2 molar, 10 Eq, 329 μmol) was then added dropwise and the reaction mixture was stirred for 2 h at 100° C. under N₂. The reaction was cooled to RT, then 1 N HCl (3 mL) was added. The mixture was extracted with DCM (2×5 mL), the organic phase collected, dried (phase separator) and evaporated under reduced pressure. The crude product was purified by prep-HPLC (basic method) to afford the title compound (6 mg) as a clear white solid. ¹H NMR (400 MHz, DMSO-d6) δ 11.47 (s, 1H), 8.65-8.58 (m, 2H), 8.56 (s, 1H), 8.44 (d, J=8.4 Hz, 1H), 8.30 (dd, J=8.4, 1.9 Hz, 1H), 8.02 (s, 1H), 7.31 (dd, J=6.5, 2.0 Hz, 1H), 7.02 (dd, J=6.8, 1.9 Hz, 1H), 6.08 (t, J=6.6 Hz, 1H), 5.08 (s, 2H), 3.88 (s, 3H). MS (ES⁺): 398 (M+H)⁺

### Example 201—6-((3,4-dihydro-2H-benzo[b][1,4]oxazin-6-yl)sulfonyl)-2-((1-methyl-1H-pyrazol-3-yl)methyl)phthalazin-1(2H)-one

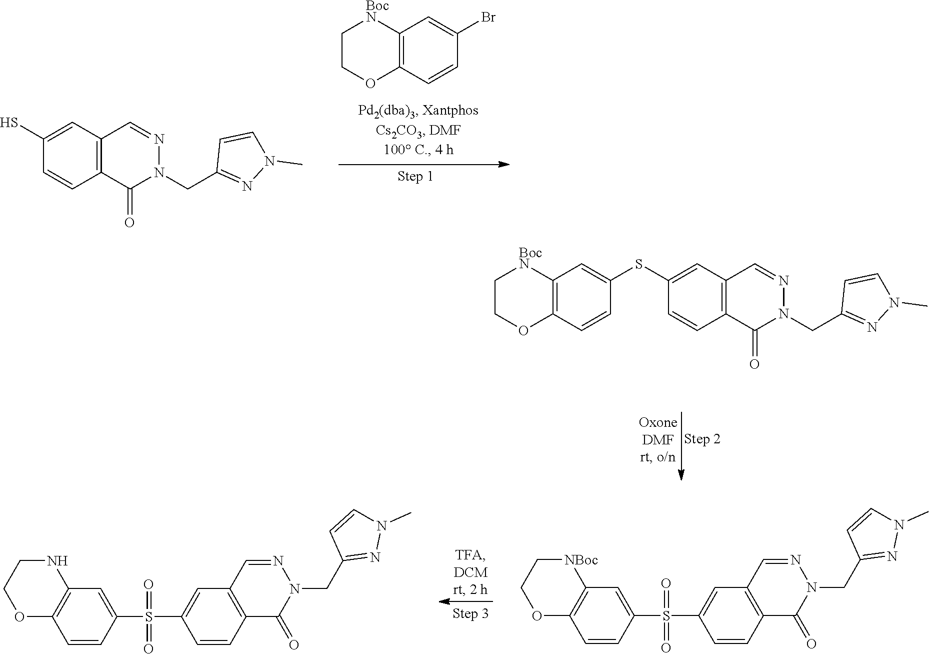 [SMILES: CC(C)(C)OC(=O)N1CCOc2ccc(Br)cc21 ; Cn1ccc(Cn2ncc3cc(S)ccc3c2=O)n1 ; Cn1ccc(Cn2ncc3cc(Sc4ccc5c(c4)N(C(=O)OC(C)(C)C)CCO5)ccc3c2=O)n1 ; Cn1ccc(Cn2ncc3cc(S(=O)(=O)c4ccc5c(c4)NCCO5)ccc3c2=O)n1 ; Cn1ccc(Cn2ncc3cc(S(=O)(=O)c4ccc5c(c4)N(C(=O)OC(C)(C)C)CCO5)ccc3c2=O)n1]

### Step 1

N₂ was bubbled through a mixture of 6-mercapto-2-((1-methyl-1H-pyrazol-3-yl)methyl)phthalazin-1(2H)-one (Intermediate 48, 50 mg, 1 Eq, 0.18 mmol), tert-butyl 6-bromo-2,3-dihydro-4H-benzo[b][1,4]oxazine-4-carboxylate (87 mg, 1.5 Eq, 0.28 mmol) and cesium carbonate (0.12 g, 2 Eq, 0.37 mmol) in DMF (1 mL) for 5 minutes. Pd₂dba₃ (1 Eq, 0.18 mmol) and Xantphos (1 Eq, 0.18 mmol) were added and the mixture was stirred at 100° C. under N₂ for 4 h, then allowed to cool to RT and stirred for 4 days. Water and DCM were added and the layers separated through a phase separator. The organic layer was washed with brine, then absorbed on silica. The crude product was purified by chromatography on silica gel. The resulting orange oil/solid was dissolved in DCM and washed with water, brine, dried (MgSO₄) and concentrated under vacuum to afford tert-butyl 6-((2-((1-methyl-1H-pyrazol-3-yl)methyl)-1-oxo-1,2-dihydrophthalazin-6-yl)thio)-2,3-dihydro-4H-benzo[b][1,4]oxazine-4-carboxylate (18 mg) as an orange solid. MS (ES⁺): 506 (M+H)⁺

### Step 2

A mixture of tert-butyl 6-((2-((1-methyl-1H-pyrazol-3-yl)methyl)-1-oxo-1,2-dihydrophthalazin-6-yl)thio)-2,3-dihydro-4H-benzo[b][1,4]oxazine-4-carboxylate (18 mg, 1 Eq, 33 μmol) and OXONE® (51 mg, 2.5 Eq, 84 μmol) in DMF (0.5 mL) was stirred overnight. Water and DCM were added and the layers separated through a phase separator. The organic layer was washed with brine, then absorbed on silica. The crude product was purified by chromatography on silica gel to afford tert-butyl 6-((2-((1-methyl-1H-pyrazol-3-yl)methyl)-1-oxo-1,2-dihydrophthalazin-6-yl)sulfonyl)-2,3-dihydro-4H-benzo[b][1,4]oxazine-4-carboxylate (8 mg) as a white solid. MS (ES⁺): 538 (M+H)⁺

### Step 3

TFA (23.9 mg, 16.2 μL, 15 Eq, 210 μmol) was added to a solution of tert-butyl 6-((2-((1-methyl-1H-pyrazol-3-yl)methyl)-1-oxo-1,2-dihydrophthalazin-6-yl)sulfonyl)-2,3-dihydro-4H-benzo[b][1,4]oxazine-4-carboxylate (8.00 mg, 94% Wt, 1 Eq, 14.0 μmol) in DCM (0.2 mL) and the mixture was stirred for 2 h, then concentrated under vacuum. Sat. NaHCO₃ and DCM were added and the layers separated through a phase separator. The organic layer was washed with brine, dried (MgSO₄) and concentrated under vacuum to afford the title compound (4.9 mg) as a white solid. ¹H NMR (DMSO-d6) δ: 8.60 (s, 1H), 8.55 (d, J=1.8 Hz, 1H), 8.42 (d, J=8.4 Hz, 1H), 8.19 (dd, J=8.5, 1.9 Hz, 1H), 7.56 (d, J=2.3 Hz, 1H), 7.14 (d, J=2.3 Hz, 1H), 7.12-7.07 (m, 1H), 6.84 (d, J=8.4 Hz, 1H), 6.39 (s, 1H), 6.09 (d, J=2.2 Hz, 1H), 5.25 (s, 2H), 4.20-4.12 (m, 2H), 3.75 (s, 3H), 3.29-3.25 (m, 2H). MS (ES⁺): 438 (M+H)⁺

### Example 202—6-((3,4-dihydro-2H-benzo[b][1,4]oxazin-6-yl)sulfonyl)-2-((1-(2-hydroxyethyl)-1H-pyrazol-3-yl)methyl)phthalazin-1(2H)-one

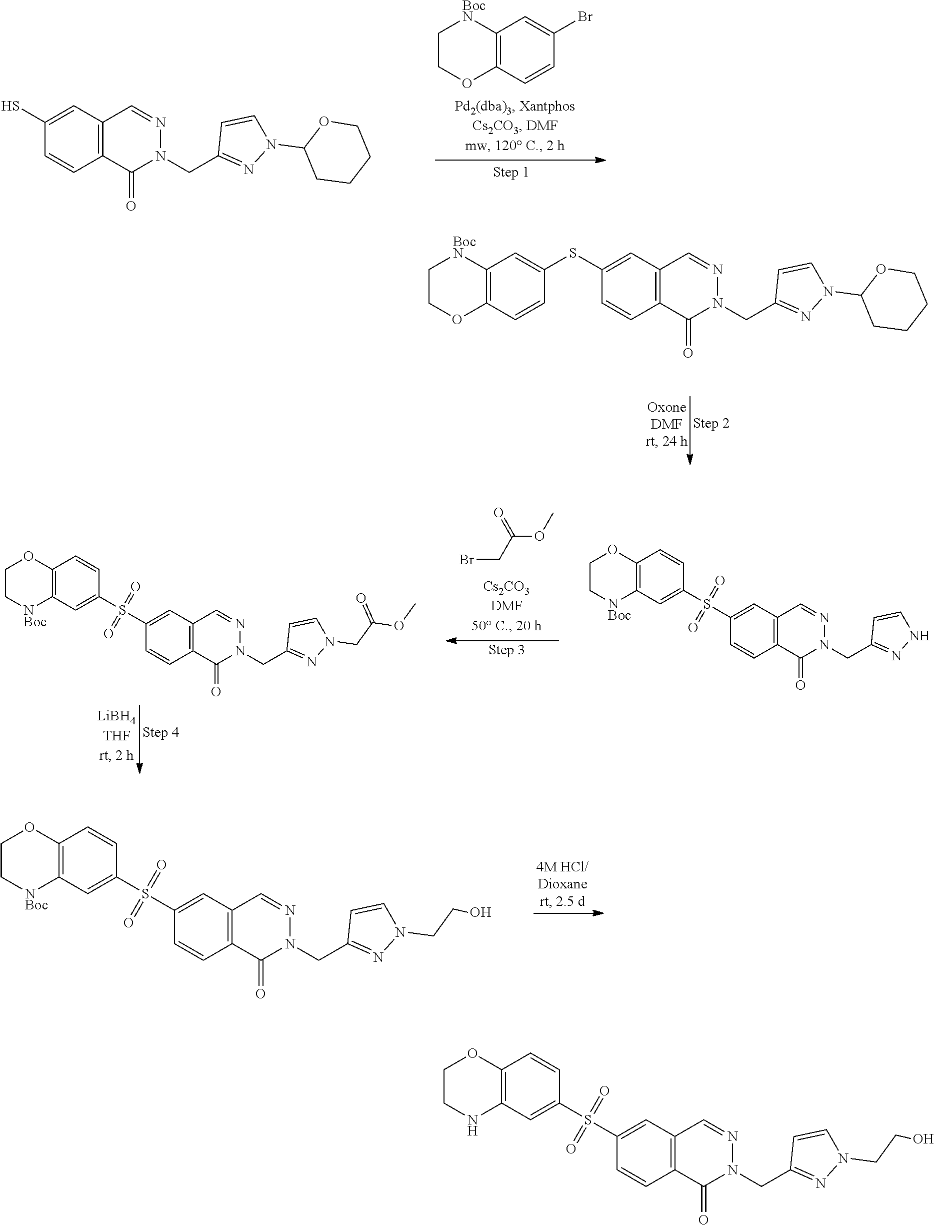

### Step 1

A suspension of 6-mercapto-2-((1-(tetrahydro-2H-pyran-2-yl)-1H-pyrazol-3-yl)methyl)phthalazin-1(2H)-one (Intermediate 49,145 mg, 95% Wt, 1 Eq, 402 μmol), cesium carbonate (262 mg, 2.0 Eq, 805 μmol), Pd₂(dba)₃ (18.4 mg, 0.05 Eq, 20.1 μmol) and Xantphos (23.3 mg, 0.10 Eq, 40.2 μmol) in DMF (2 mL) was treated with tert-butyl 6-bromo-2,3-dihydro-4H-benzo[b][1,4]oxazine-4-carboxylate (145 mg, 95% Wt, 1.09 Eq, 438 μmol). The reaction mixture was heated in the microwave at 120° C. for 2 h. More tert-butyl 6-bromo-2,3-dihydro-4H-benzo[b][1,4]oxazine-4-carboxylate (55 mg, 0.43 Eq, 175 μmol), Pd₂(dba)₃ (18.4 mg, 0.05 Eq, 20.1 μmol) and Xantphos (23.3 mg, 0.10 Eq, 40.2 μmol) were then added and the mixture heated in the microwave at 120° C. for 30 min. The reaction mixture was concentrated in vacuo to afford the crude product. The crude product was purified by chromatography on silica gel to afford a brown oil. The residue was azeotroped with diethyl ether to afford tert-butyl 6-((1-oxo-2-((1-(tetrahydro-2H-pyran-2-yl)-1H-pyrazol-3-yl)methyl)-1,2-dihydrophthalazin-6-yl)thio)-2,3-dihydro-4H-benzo[b][1,4]oxazine-4-carboxylate (202 mg) as a yellow foam. MS (ES⁺): 576 (M+H)⁺

### Step 2

A suspension of tert-butyl 6-((1-oxo-2-((1-(tetrahydro-2H-pyran-2-yl)-1H-pyrazol-3-yl)methyl)-1,2-dihydrophthalazin-6-yl)thio)-2,3-dihydro-4H-benzo[b][1,4]oxazine-4-carboxylate (200 mg, 90% Wt, 1 Eq, 313 μmol) and OXONE® (423 mg, 2.2 Eq, 688 μmol) in DMF (2 mL) was stirred at RT for 24 h. The reaction mixture was diluted with DCM (5 mL) and washed with water (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford tert-butyl 6-((1-oxo-2-((1-(tetrahydro-2H-pyran-2-yl)-1H-pyrazol-3-yl)methyl)-1,2-dihydrophthalazin-6-yl)sulfonyl)-2,3-dihydro-4H-benzo[b][1,4]oxazine-4-carboxylate (95 mg) as a yellow solid after trituration with diethyl ether. MS (ES⁺): 608 (M+H)⁺

tert-Butyl 6-((2-((1H-pyrazol-3-yl)methyl)-1-oxo-1,2-dihydrophthalazin-6-yl)sulfonyl)-2,3-dihydro-4H-benzo[b][1,4]oxazine-4-carboxylate (35 mg) was also isolated from the column as a yellow solid after trituration with diethyl ether. MS (ES⁺): 524 (M+H)⁺

### Step 3

A suspension of tert-butyl 6-((2-((1H-pyrazol-3-yl)methyl)-1-oxo-1,2-dihydrophthalazin-6-yl)sulfonyl)-2,3-dihydro-4H-benzo[b][1,4]oxazine-4-carboxylate (35 mg, 83% Wt, 1 Eq, 55 μmol) and cesium carbonate (36 mg, 2.0 Eq, 0.11 mmol) in DMF (2 mL) was treated with methyl 2-bromoacetate (16.6 mg, 10.0 μL, 2.0 Eq, 109 μmol). The reaction mixture was stirred at 50° C. for 20 h. The reaction mixture was diluted with DCM (5 mL) and washed with sat. aq NaHCO₃ (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford a mixture of tert-butyl 6-((2-((1-(2-methoxy-2-oxoethyl)-1H-pyrazol-3-yl)methyl)-1-oxo-1,2-dihydrophthalazin-6-yl)sulfonyl)-2,3-dihydro-4H-benzo[b][1,4]oxazine-4-carboxylate (46 mg) and tert-butyl 6-((2-((1-(2-methoxy-2-oxoethyl)-1H-pyrazol-5-yl)methyl)-1-oxo-1,2-dihydrophthalazin-6-yl)sulfonyl)-2,3-dihydro-4H-benzo[b][1,4]oxazine-4-carboxylate (46 mg) as a clear yellow oil that solidified on standing. MS (ES⁺): 596 (M+H)⁺

### Step 4

A stirred solution of tert-butyl 6-((2-((1-(2-methoxy-2-oxoethyl)-1H-pyrazol-3-yl)methyl)-1-oxo-1,2-dihydrophthalazin-6-yl)sulfonyl)-2,3-dihydro-4H-benzo[b][1,4]oxazine-4-carboxylate (46 mg, 65% Wt, 1 Eq, 50 μmol) and tert-butyl 6-((2-((1-(2-methoxy-2-oxoethyl)-1H-pyrazol-5-yl)methyl)-1-oxo-1,2-dihydrophthalazin-6-yl)sulfonyl)-2,3-dihydro-4H-benzo[b][1,4]oxazine-4-carboxylate (46 mg, 28% Wt, 0.43 Eq, 22 μmol) in THF (1 mL) was treated with lithium borohydride (3.5 mg, 80 μL, 2.00 molar, 3.2 Eq, 0.16 mmol) at RT. The reaction mixture was stirred at RT for 2 h. DCM (5 mL) and sat. aq. NaHCO₃ (5 mL) were added and the organic layer was collected. The aqueous was extracted with DCM (5 mL) and the combined organic extracts were dried (phase separator) and concentrated in vacuo to afford the crude product. The crude product was purified by chromatography on silica gel to afford tert-butyl 6-((2-((1-(2-hydroxyethyl)-1H-pyrazol-3-yl)methyl)-1-oxo-1,2-dihydrophthalazin-6-yl)sulfonyl)-2,3-dihydro-4H-benzo[b][1,4]oxazine-4-carboxylate (15 mg) as a pale yellow solid. MS (ES⁺): 568 (M+H)⁺

### Step 5

A solution of tert-butyl 6-((2-((1-(2-hydroxyethyl)-1H-pyrazol-3-yl)methyl)-1-oxo-1,2-dihydrophthalazin-6-yl)sulfonyl)-2,3-dihydro-4H-benzo[b][1,4]oxazine-4-carboxylate (15 mg, 76% Wt, 1 Eq, 20 μmol) and tert-butyl 6-((2-((1-(2-hydroxyethyl)-1H-pyrazol-5-yl)methyl)-1-oxo-1,2-dihydrophthalazin-6-yl)sulfonyl)-2,3-dihydro-4H-benzo[b][1,4]oxazine-4-carboxylate (15 mg, 21% Wt, 0.28 Eq, 5.5 μmol) in DCM (0.5 mL) was treated with HCl (4 M in dioxane) (210 mg, 200 μL, 4.00 molar, 40 Eq, 800 μmol). The reaction mixture was stirred at room temperature for 65 h. The reaction mixture was diluted with DCM (5 mL) and washed with sat. aq. NaHCO₃ (5 mL). The organic layer was collected and the aqueous was extracted with DCM (5 mL). The combined organic extracts were dried (phase separator) and concentrated in vacuo to afford the crude product. The crude product was purified by preparative HPLC (Method B, basic method) to afford the title compound (4.3 mg) as a light yellow solid. ¹H NMR (400 MHz, DMSO-d6) δ 8.61 (s, 1H), 8.55 (d, J=1.8 Hz, 1H), 8.43 (d, J=8.4 Hz, 1H), 8.19 (dd, J=8.4, 1.9 Hz, 1H), 7.58 (d, J=2.3 Hz, 1H), 7.14 (d, J=2.3 Hz, 1H), 7.09 (dd, J=8.4, 2.3 Hz, 1H), 6.84 (d, J=8.3 Hz, 1H), 6.41-6.37 (m, 1H), 6.07 (d, J=2.2 Hz, 1H), 5.26 (s, 2H), 4.83 (t, J=5.3 Hz, 1H), 4.16 (t, J=4.3 Hz, 2H), 4.05 (t, J=5.6 Hz, 2H), 3.67 (q, J=5.6 Hz, 2H), 3.29-3.25 (m, 2H). MS (ES⁺): 468 (M+H)⁺

### Example 203—2-((1H-pyrazol-3-yl)methyl)-6-((2,3-dihydrobenzofuran-5-yl)sulfonyl)phthalazin-1(2H)-one

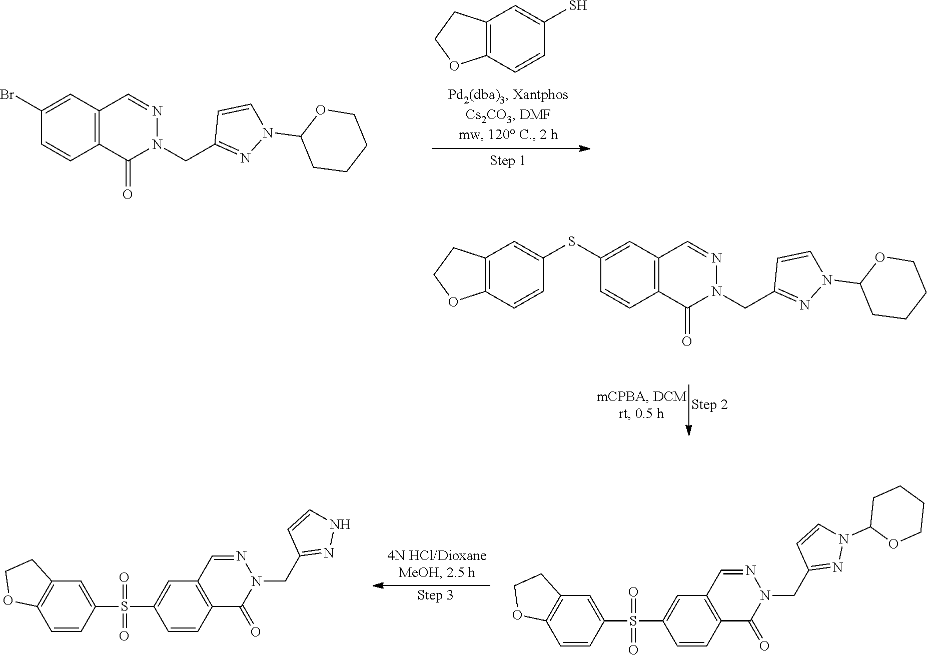 [SMILES: Sc1ccc2c(c1)CCO2 ; O=c1c2ccc(Br)cc2cnn1Cc1ccn(C2CCCCO2)n1 ; O=c1c2ccc(Sc3ccc4c(c3)CCO4)cc2cnn1Cc1ccn(C2CCCCO2)n1 ; O=c1c2ccc(S(=O)(=O)c3ccc4c(c3)CCO4)cc2cnn1Cc1cc[nH]n1 ; O=c1c2ccc(S(=O)(=O)c3ccc4c(c3)CCO4)cc2cnn1Cc1ccn(C2CCCCO2)n1]

### Step 1

N₂ was bubbled through a mixture of 6-bromo-2-((1-(tetrahydro-2H-pyran-2-yl)-1H-pyrazol-3-yl)methyl)phthalazin-1(2H)-one (Intermediate 49A, 100 mg, 1 Eq, 257 μmol), 2,3-dihydrobenzofuran-5-thiol (39.1 mg, 1 Eq, 257 μmol) and cesium carbonate (167 mg, 2 Eq, 514 μmol) in DMF (1.5 mL) for 5 minutes. Pd₂dba₃ (11.8 mg, 0.05 Eq, 12.8 μmol) and Xantphos (14.9 mg, 0.1 Eq, 25.7 μmol) were added and the mixture was stirred at 100° C. under N₂ for 2.5 h, then allowed to cool to RT. Water and DCM were added and the layers separated through a phase separator. The organic layer was washed with brine, then absorbed on silica. The crude product was purified by chromatography on silica gel to afford 6-((2,3-dihydrobenzofuran-5-yl)thio)-2-((1-(tetrahydro-2H-pyran-2-yl)-1H-pyrazol-3-yl)methyl)phthalazin-1(2H)-one (69 mg) as a pale yellow solid. MS (ES⁺): 461 (M+H)⁺

### Step 2

mCPBA (86 mg, 70% Wt, 2.5 Eq, 0.35 mmol) was added to a solution of 6-((2,3-dihydrobenzofuran-5-yl)thio)-2-((1-(tetrahydro-2H-pyran-2-yl)-1H-pyrazol-3-yl)methyl)phthalazin-1(2H)-one (69 mg, 93% Wt, 1 Eq, 0.14 mmol) in DCM (1 mL) and the mixture was stirred for 30 minutes. 2 N NaOH, Sat. NaHCO₃ and DCM were added and the layers separated through a phase separator. The organic layer was washed with brine, dried (MgSO₄) and concentrated under vacuum to afford crude 6-((2,3-dihydrobenzofuran-5-yl)sulfonyl)-2-((1-(tetrahydro-2H-pyran-2-yl)-1H-pyrazol-3-yl)methyl)phthalazin-1(2H)-one (69 mg) as an off-white solid. MS (ES⁺): 493 (M+H)⁺.

### Step 3

HCl (4 N in dioxane) (72 mg, 0.49 mL, 4 molar, 15 Eq, 2.0 mmol) was added to a solution of 6-((2,3-dihydrobenzofuran-5-yl)sulfonyl)-2-((1-(tetrahydro-2H-pyran-2-yl)-1H-pyrazol-3-yl)methyl)phthalazin-1(2H)-one (69 mg, 94% Wt, 1 Eq, 0.13 mmol) in MeOH (1 mL) and the mixture was stirred for 2.5 h, then concentrated under vacuum. Sat. NaHCO₃ and DCM were added and the layers separated through a phase separator. The organic layer was washed with brine, then absorbed on silica. The crude product was purified by chromatography on silica gel to afford the title compound (36.5 mg) as a white solid. ¹H NMR (DMSO-d6) δ: 12.64 (s, 1H), 8.61 (d, J=1.8 Hz, 1H), 8.59 (s, 1H), 8.42 (d, J=8.4 Hz, 1H), 8.26 (dd, J=8.4, 1.9 Hz, 1H), 7.86 (d, J=2.0 Hz, 1H), 7.79 (dd, J=8.5, 2.2 Hz, 1H), 7.63-7.58 (m, 1H), 6.98 (d, J=8.5 Hz, 1H), 6.12 (s, 1H), 5.29 (s, 2H), 4.64 (t, J=8.8, 8.8 Hz, 2H), 3.24 (t, J=8.8, 8.8 Hz, 2H). MS (ES⁺): 409 (M+H)⁺

### Example 204—6-((1-methyl-1H-pyrazol-4-yl)sulfonyl)-2-((6-methylpyridin-2-yl)methyl)phthalazin-1(2H)-one

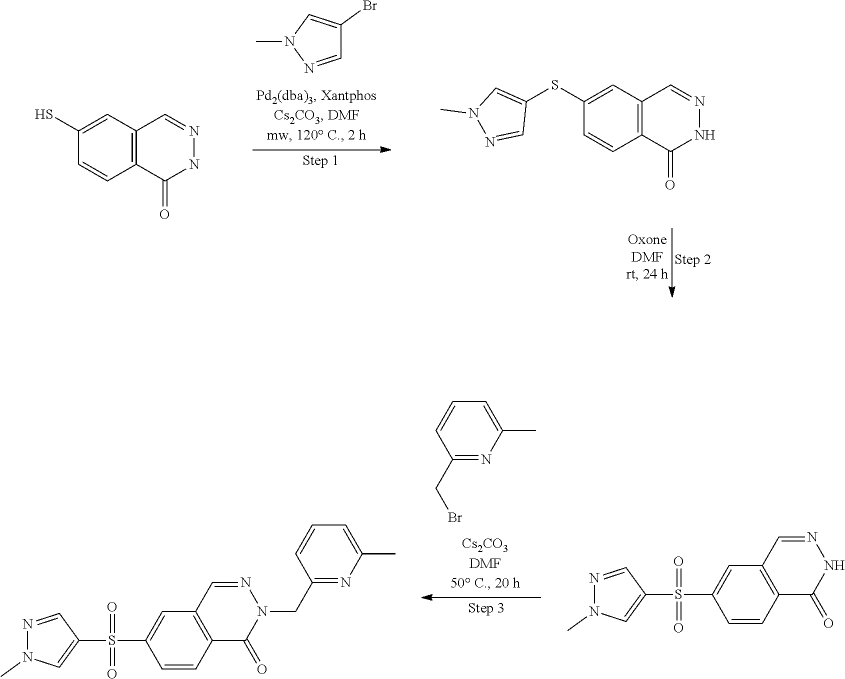

### Step 1

N₂ was bubbled through a mixture of 6-mercaptophthalazin-1(2H)-one (Intermediate 53, 80 mg, 1 Eq, 0.45 mmol), 4-iodo-1-methyl-1H-pyrazole (93 mg, 1 Eq, 0.45 mmol) and cesium carbonate (0.29 g, 2 Eq, 0.90 mmol) in DMF (3 mL) for 5 minutes. Pd₂dba₃ (21 mg, 0.05 Eq, 22 μmol) and Xantphos (26 mg, 0.1 Eq, 45 μmol) were added and the mixture was stirred at 120° C. under microwave radiation for 2 h, then allowed to cool to RT. 1 N HCl (5 mL), water and DCM were added and the layers separated through a phase separator. The organic layer was washed with brine, then absorbed on silica. The crude product was purified by chromatography on silica gel to afford 6-((1-methyl-1H-pyrazol-4-yl)thio)phthalazin-1(2H)-one (80 mg, 1 Eq, 0.45 mmol) as a red solid. MS (ES⁺): 259 (M+H)⁺

### Step 2

OXONE® (90 mg, 3 Eq, 0.15 mmol) was added to a solution of 6-((1-methyl-1H-pyrazol-4-yl)thio)phthalazin-1(2H)-one (17 mg, 74% Wt, 1 Eq, 49 μmol) in DMF (6.00 mL) and the reaction mixture was left to stir for 20 h at RT. The reaction was diluted with water (30 mL) and extracted with DCM (3×10 mL). The combined organic layers were dried over MgSO₄, filtered and concentrated in vacuo. The resultant residue was twice taken up in toluene (5 mL) and concentrated in vacuo to afford crude product as a yellow solid. The crude product was purified by chromatography on silica gel to afford 6-((1-methyl-1H-pyrazol-4-yl)sulfonyl)phthalazin-1(2H)-one (13 mg) as a yellow solid. MS (ES⁺): 291 (M+H)⁺

### Step 3

6-((1-methyl-1H-pyrazol-4-yl)sulfonyl)phthalazin-1(2H)-one (13 mg, 100% Wt, 1.0 Eq, 45 μmol), 2-(bromomethyl)-6-methylpyridine (12 mg, 1.5 Eq, 67 μmol), cesium carbonate (29 mg, 2.0 Eq, 90 μmol) and DMF (1 mL) were combined and stirred under N₂ at RT for 30 minutes. The mixture was diluted with water (30 mL) and extracted with DCM (3×10 mL). The combined organic fractions were dried over MgSO₄, filtered and concentrated in vacuo. The resulting residue was diluted with toluene (5 mL) and concentrated in vacuo to afford crude product. The crude product was purified by chromatography on silica gel to afford the title compound (1.6 mg) as a white solid. ¹H NMR (400 MHz, DMSO-d6) δ 8.66-8.60 (m, 2H), 8.56 (s, 1H), 8.44 (d, J=8.4 Hz, 1H), 8.31 (dd, J=8.4, 1.9 Hz, 1H), 8.03 (s, 1H), 7.59 (t, J=7.7 Hz, 1H), 7.13 (d, J=7.7 Hz, 1H), 6.95 (d, J=7.7 Hz, 1H), 5.39 (s, 2H), 3.88 (s, 3H), 2.40 (s, 3H). MS (ES⁺): 396 (M+H)⁺

### Example 205—2-((1-cyclopropyl-1H-pyrazol-3-yl)methyl)-6-((6-methoxypyridin-3-yl)sulfonyl)phthalazin-1(2H)-one

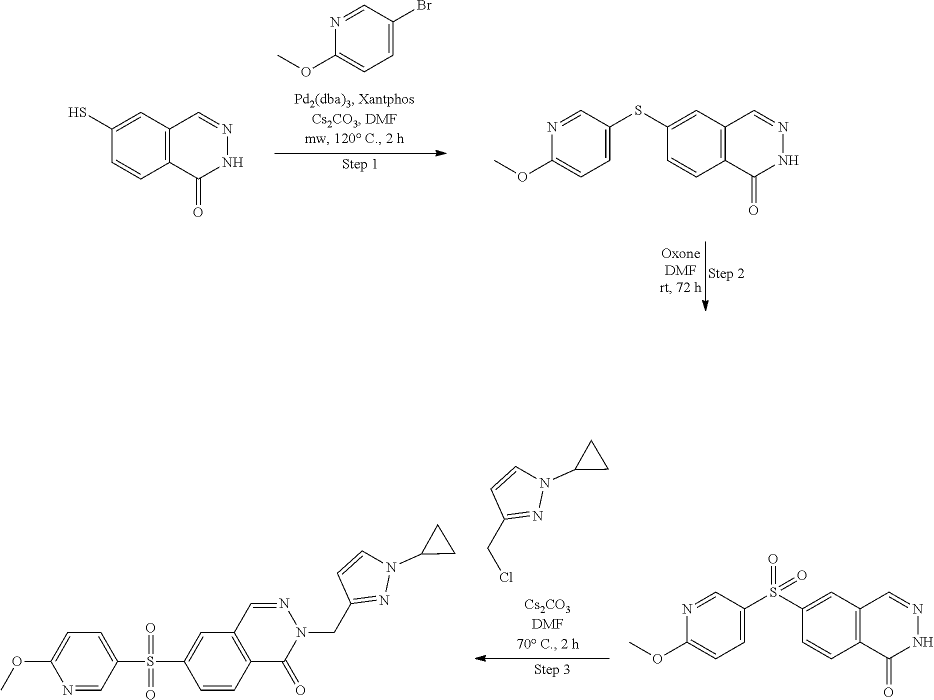

### Step 1

A mixture of 6-mercaptophthalazin-1(2H)-one (Intermediate 53, 150.0 mg, 1 Eq, 841.7 μmol), Pd2(dba)3 (38.54 mg, 0.05 Eq, 42.09 μmol), Xantphos (48.70 mg, 0.1 Eq, 84.17 μmol), 5-bromo-2-methoxypyridine (158.3 mg, 108.9 μL, 1 Eq, 841.7 μmol), and cesium carbonate (548.5 mg, 2 Eq, 1.683 mmol) in DMF (16 mL) was heated to 120° C. for 2 h under microwave irradiation. The reaction mixture was cooled to RT and adsorbed on silica gel. The crude product was purified by chromatography on silica gel to afford 6-((6-methoxypyridin-3-yl)thio)phthalazin-1(2H)-one (25 mg) as a light yellow oil. MS (ES⁺): 286 (M+H)⁺

### Step 2

OXONE® (0.12 g, 3 Eq, 0.19 mmol) was added to a solution of 6-((6-methoxypyridin-3-yl)thio)phthalazin-1(2H)-one (25 mg, 74% Wt, 1 Eq, 65 μmol) in DMF (3 mL) and the reaction mixture was left to stir for 72 h at RT. The reaction mixture was diluted with DCM and adsorbed on silica gel. The crude product was purified by chromatography on silica gel to afford 6-((6-methoxypyridin-3-yl)sulfonyl)phthalazin-1(2H)-one (22 mg) as a clear white solid. MS (ES⁺): 318 (M+H)⁺

### Step 3

Cesium carbonate (36 mg, 2.0 Eq, 0.11 mmol) was added to a stirred solution of 6-((6-methoxypyridin-3-yl)sulfonyl)phthalazin-1(2H)-one (22 mg, 79% Wt, 1 Eq, 55 μmol) in dry DMF (2 mL). The reaction mixture was stirred for 5 minutes, then 3-(chloromethyl)-1-cyclopropyl-1H-pyrazole (Intermediate 6, 8.6 mg, 1 Eq, 55 μmol) was added. The resulting mixture was stirred at 70° C. for 2 h. The reaction mixture was cooled to RT, diluted with DCM and adsorbed on silica gel. The crude product was purified by prep-HPLC (Method A, acidic method) to afford the title compound (3 mg) as a white solid. ¹H NMR (400 MHz, DMSO-d6) δ 8.86 (d, J=2.6 Hz, 1H), 8.68 (d, J=1.8 Hz, 1H), 8.58 (s, 1H), 8.43 (d, J=8.4 Hz, 1H), 8.34 (dd, J=8.5, 1.8 Hz, 1H), 8.23 (dd, J=8.8, 2.6 Hz, 1H), 7.64 (d, J=2.3 Hz, 1H), 7.04 (d, J=8.9 Hz, 1H), 6.06 (d, J=2.3 Hz, 1H), 5.24 (s, 2H), 3.94 (s, 3H), 3.67-3.56 (m, 1H), 0.99-0.93 (m, 2H), 0.93-0.85 (m, 2H). MS (ES⁺): 438 (M+H)⁺

### Example 206—6-((2,3-dihydrobenzo[b][1,4]dioxin-6-yl)sulfonyl)-2-((1-(2-hydroxyethyl)-1H-pyrazol-3-yl)methyl)phthalazin-1(2H)-one; and

### Example 207—2-((1H-pyrazol-3-yl)methyl)-6-((2,3-dihydrobenzo[b][1,4]dioxin-6-yl)sulfonyl)phthalazine-1(2H)-one

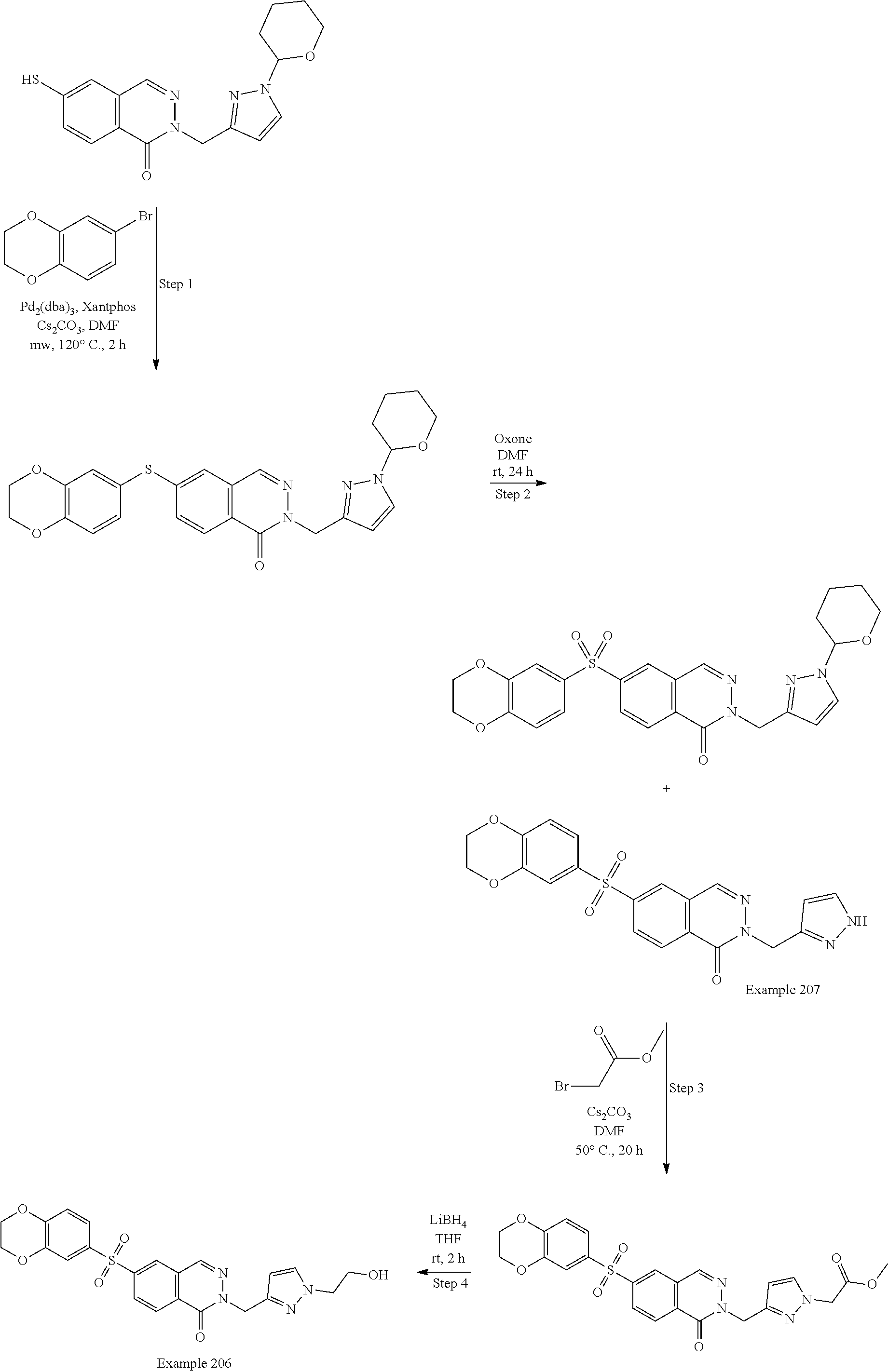

### Step 1

A suspension of 6-mercapto-2-((1-(tetrahydro-2H-pyran-2-yl)-1H-pyrazol-3-yl)methyl)phthalazin-1(2H)-one (Intermediate 49, 150 mg, 95% Wt, 1 Eq, 416 μmol), cesium carbonate (271 mg, 2.0 Eq, 832 μmol), Pd2(dba)3 (19.1 mg, 0.05 Eq, 20.8 μmol) and Xantphos (24.1 mg, 0.10 Eq, 41.6 μmol) in DMF (2 mL) was treated with 6-bromo-2,3-dihydrobenzo[b][1,4]dioxine (0.11 g, 68 μL, 1.2 Eq, 0.51 mmol). The reaction mixture was heated in the microwave at 120° C. for 2 h. 6-bromo-2,3-dihydrobenzo[b][1,4]dioxine (0.11 g, 68 μL, 1.2 Eq, 0.51 mmol), Pd₂(dba)₃ (19.1 mg, 0.05 Eq, 20.8 μmol) and Xantphos (24.1 mg, 0.10 Eq, 41.6 μmol) were added and the mixture was heated in the microwave at 120° C. for 1 h. The reaction mixture was filtered through a phase separator, washing with EtOAc and then concentrated in vacuo. The residue was azeotroped with toluene (2 times) to afford the crude product. The crude product was purified by chromatography on silica gel to afford 6-((2,3-dihydrobenzo[b][1,4]dioxin-6-yl)thio)-2-((1-(tetrahydro-2H-pyran-2-yl)-1H-pyrazol-3-yl)methyl)phthalazin-1(2H)-one (233 mg) as a brown oil. MS (ES⁺): 477 (M+H)⁺

### Step 2

A suspension of 6-((2,3-dihydrobenzo[b][1,4]dioxin-6-yl)thio)-2-((1-(tetrahydro-2H-pyran-2-yl)-1H-pyrazol-3-yl)methyl)phthalazin-1(2H)-one (207 mg, 96% Wt, 1 Eq, 0.416 mmol) and OXONE® (563 mg, 2.2 Eq, 915 μmol) in DMF (2 mL) was stirred at RT for 24 h. The reaction mixture was diluted with DCM (10 mL) and washed with water (10 mL). The organic layer was collected and the aqueous was extracted with DCM (2×5 mL). The combined organic extracts were dried (phase separator) and concentrated in vacuo to afford the crude product. The crude product was purified by chromatography on silica gel to afford 6-((2,3-dihydrobenzo[b][1,4]dioxin-6-yl)sulfonyl)-2-((1-(tetrahydro-2H-pyran-2-yl)-1H-pyrazol-3-yl)methyl)phthalazin-1(2H)-one (65 mg) as a yellow solid after trituration with diethyl ether. MS (ES⁺): 509 (M+H)⁺2-((1H-pyrazol-3-yl)methyl)-6-((2,3-dihydrobenzo[b][1,4]dioxin-6-yl)sulfonyl)phthalazin-1(2H)-one (Example 210) (51 mg) was also isolated from the column as a yellow solid after trituration with diethyl ether. ¹H NMR (400 MHz, DMSO-d6) δ 12.65 (s, 1H), 8.65 (d, J=1.8 Hz, 1H), 8.58 (s, 1H), 8.41 (d, J=8.4 Hz, 1H), 8.29 (dd, J=8.4, 1.8 Hz, 1H), 7.62 (s, 1H), 7.48 (dq, J=4.7, 2.3 Hz, 2H), 7.12-7.07 (m, 1H), 6.12 (s, 1H), 5.30 (s, 2H), 4.35-4.30 (m, 2H), 4.31-4.26 (m, 2H). MS (ES⁺): 425 (M+H)⁺

### Step 3

A suspension of 2-((1H-pyrazol-3-yl)methyl)-6-((2,3-dihydrobenzo[b][1,4]dioxin-6-yl)sulfonyl)phthalazin-1(2H)-one (30.0 mg, 95% Wt, 1 Eq, 67.1 μmol) and cesium carbonate (43.8 mg, 2.0 Eq, 134 μmol) in DMF (2 mL) was treated with methyl 2-bromoacetate (16.6 mg, 10.0 μL, 1.62 Eq, 109 μmol). The reaction mixture was stirred at 50° C. for 20 h, then allowed to cool to RT. The reaction mixture was diluted with DCM (5 mL) and washed with sat. aq. NaHCO₃ (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford methyl 2-(3-((6-((2,3-dihydrobenzo[b][1,4]dioxin-6-yl)sulfonyl)-1-oxophthalazin-2(1H)-yl)methyl)-1H-pyrazol-1-yl)acetate (17 mg) as a pale yellow solid. MS (ES⁺): 497 (M+H)⁺

### Step 4

A stirred solution of methyl 2-(3-((6-((2,3-dihydrobenzo[b][1,4]dioxin-6-yl)sulfonyl)-1-oxophthalazin-2(1H)-yl)methyl)-1H-pyrazol-1-yl)acetate (19 mg, 98% Wt, 1 Eq, 38 μmol) in THF (1 mL) was treated with lithium borohydride (1.7 mg, 40 μL, 2.00 molar, 2.1 Eq, 80 μmol) at RT and the mixture was stirred for 2 h. DCM (5 mL) and sat. aq. NaHCO₃ (5 mL) were added and the organic layer was collected. The aqueous was extracted with DCM (5 mL) and the combined organic extracts were dried (phase separator) and concentrated in vacuo to afford the crude product. The crude product was purified by preparative HPLC (Method B, basic method) to afford the title compound (4.2 mg) as a white solid. ¹H NMR (400 MHz, DMSO-d6) δ 8.64 (d, J=1.8 Hz, 1H), 8.58 (s, 1H), 8.41 (d, J=8.4 Hz, 1H), 8.29 (dd, J=8.5, 1.9 Hz, 1H), 7.58 (d, J=2.2 Hz, 1H), 7.52-7.45 (m, 2H), 7.10 (d, J=9.1 Hz, 1H), 6.07 (d, J=2.2 Hz, 1H), 5.26 (s, 2H), 4.83 (t, J=5.3 Hz, 1H), 4.30 (ddt, J=8.4, 6.6, 3.1 Hz, 4H), 4.05 (t, J=5.7 Hz, 2H), 3.67 (q, J=5.6 Hz, 2H). MS (ES⁺): 469 (M+H)⁺

### Example 208—6-((2,3-dihydrobenzofuran-5-yl)sulfonyl)-2-((4-methyl-1H-pyrazol-3-yl)methyl)phthalazin-1(2H)-one

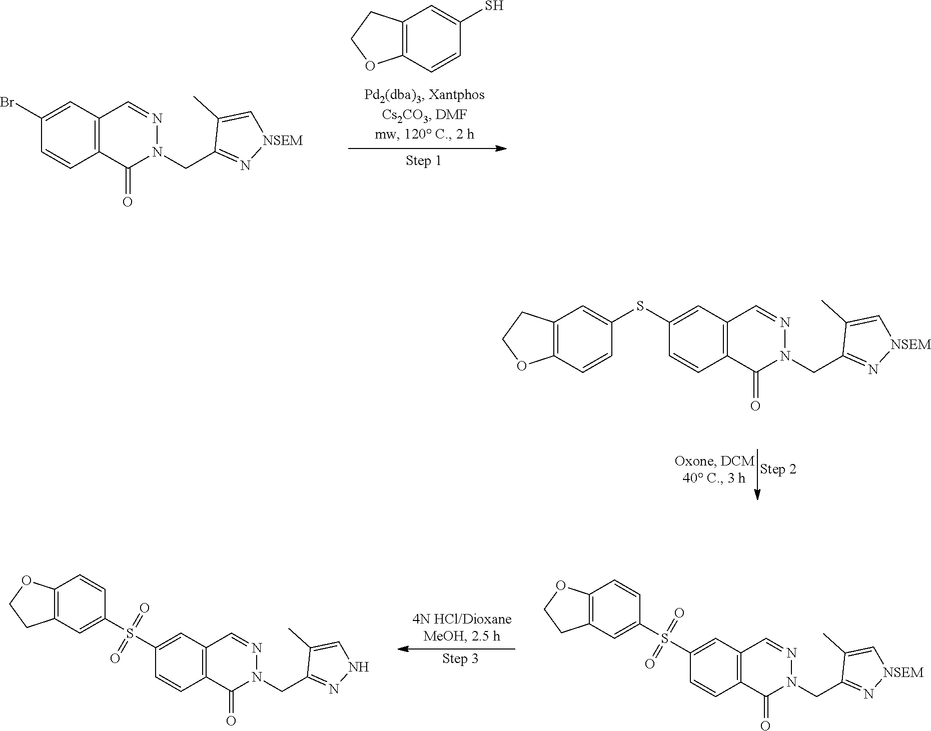 [SMILES: Sc1ccc2c(c1)CCO2 ; Cc1cn(COCC[Si](C)(C)C)nc1Cn1ncc2cc(Br)ccc2c1=O ; Cc1cn(COCC[Si](C)(C)C)nc1Cn1ncc2cc(Sc3ccc4c(c3)CCO4)ccc2c1=O ; Cc1c[nH]nc1Cn1ncc2cc(S(=O)(=O)c3ccc4c(c3)CCO4)ccc2c1=O ; Cc1cn(COCC[Si](C)(C)C)nc1Cn1ncc2cc(S(=O)(=O)c3ccc4c(c3)CCO4)ccc2c1=O]

### Step 1

A suspension of 6-bromo-2-((4-methyl-1-((2-(trimethylsilyl)ethoxy)methyl)-1H-pyrazol-3-yl)methyl)phthalazin-1(2H)-one (Intermediate 51, 120 mg, 98% Wt, 1 Eq, 262 μmol), 2,3-dihydrobenzofuran-5-thiol (48.1 mg, 91% Wt, 1.1 Eq, 288 μmol), cesium carbonate (128 mg, 1.5 Eq, 393 μmol) in DMF (2 mL) was sparged with N₂ for 5 minutes. xantphos (15.1 mg, 0.10 Eq, 26.2 μmol) and Pd₂(dba)₃ (12.0 mg, 0.05 Eq, 13.1 μmol) were added and the reaction mixture was heated in the microwave at 120° C. for 2 h. The reaction mixture was concentrated in vacuo to afford the crude product. The crude product was purified by chromatography on silica gel to afford a mixture of 6-((2,3-dihydrobenzofuran-5-yl)thio)-2-((4-methyl-1-((2-(trimethylsilyl)ethoxy)methyl)-1H-pyrazol-3-yl)methyl)phthalazin-1(2H)-one (145 mg) and 6-bromo-2-((4-methyl-1-((2-(trimethylsilyl)ethoxy)methyl)-1H-pyrazol-3-yl)methyl)phthalazin-1(2H)-one (145 mg) as a clear orange oil. MS (ES⁺): 521 (M+H)⁺

### Step 2

A stirred solution of 6-((2,3-dihydrobenzofuran-5-yl)thio)-2-((4-methyl-1-((2-(trimethylsilyl)ethoxy)methyl)-1H-pyrazol-3-yl)methyl)phthalazin-1(2H)-one (145 mg, 68% Wt, 1 Eq, 189 μmol) and 6-bromo-2-((4-methyl-1-((2-(trimethylsilyl)ethoxy)methyl)-1H-pyrazol-3-yl)methyl)phthalazin-1(2H)-one (145 mg, 31% Wt, 0.528 Eq, 100 μmol) in DMF (3 mL) was treated with OXONE® (291 mg, 2.5 Eq, 473 μmol). The reaction mixture was stirred at RT for 20 h. Additional OXONE® (291 mg, 2.5 Eq, 473 μmol) was added and the mixture was warmed to 40° C. for 3 h. The reaction mixture was diluted with DCM (5 mL) and washed with water (10 mL). The organic layer was collected and the aqueous was extracted with DCM (2×5 mL). The combined organic extracts were dried (phase separator) and concentrated in vacuo to afford the crude product. The crude product was purified by chromatography on silica gel to afford 6-((2,3-dihydrobenzofuran-5-yl)sulfonyl)-2-((4-methyl-1-((2-(trimethylsilyl)ethoxy)methyl)-1H-pyrazol-3-yl)methyl)phthalazin-1(2H)-one (113 mg) as a clear yellow oil. MS (ES⁺): 553 (M+H)⁺

### Step 3

A solution of 6-((2,3-dihydrobenzofuran-5-yl)sulfonyl)-2-((4-methyl-1-((2-(trimethylsilyl)ethoxy)methyl)-1H-pyrazol-3-yl)methyl)phthalazin-1(2H)-one (113 mg, 95% Wt, 1 Eq, 194 μmol) in THF (2 mL) was treated with HCl (4 M in dioxane) (745 mg, 500 μL, 4.00 molar, 10.3 Eq, 2.00 mmol). The reaction mixture was stirred at 40° C. for 3 h and then allowed to cool to RT for 18 h. Additional HCl (4 M in dioxane) (745 mg, 500 μL, 4.00 molar, 10.3 Eq, 2.00 mmol) was added and stirred at 40° C. for 3 h. The reaction mixture was diluted with DCM (5 mL) and carefully washed with sat. aq. NaHCO₃ (10 mL). The organic layer was collected and the aqueous was extracted with DCM (2×5 mL). The combined organic extracts were dried (phase separator) and concentrated in vacuo to afford the crude product. The crude product was purified by chromatography on silica gel to afford the title compound (42.3 mg) as a white solid, after drying in vacuo (45° C., 20 h). ¹H NMR (400 MHz, DMSO-d6) δ 12.35 (s, 1H), 8.60 (s, 1H), 8.57 (d, J=2.4 Hz, 1H), 8.41 (d, J=8.5 Hz, 1H), 8.25 (dd, J=8.5, 1.8 Hz, 1H), 7.88-7.84 (m, 1H), 7.79 (dd, J=8.5, 2.1 Hz, 1H), 7.41 (s, 1H), 6.98 (d, J=8.5 Hz, 1H), 5.32-5.23 (m, 2H), 4.64 (t, J=8.8 Hz, 2H), 3.24 (t, J=8.8 Hz, 2H), 1.99-1.90 (m, 3H). MS (ES⁺): 423 (M+H)⁺

### Example 209—6-((2,3-dihydrobenzofuran-5-yl)sulfonyl)-2-((5-methyl-1H-pyrazol-3-yl)methyl)phthalazin-1(2H)-one

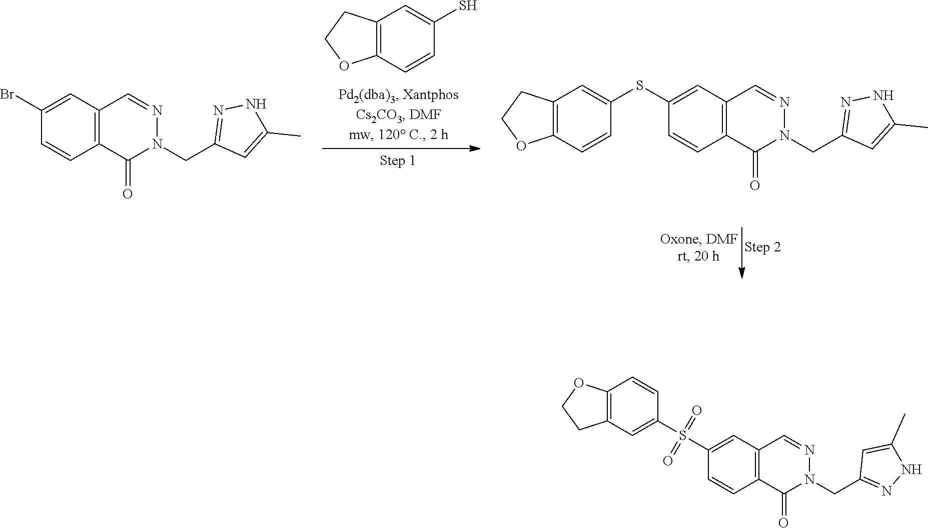 [SMILES: Sc1ccc2c(c1)CCO2 ; Cc1cc(Cn2ncc3cc(Br)ccc3c2=O)n[nH]1 ; Cc1cc(Cn2ncc3cc(Sc4ccc5c(c4)CCO5)ccc3c2=O)n[nH]1 ; Cc1cc(Cn2ncc3cc(S(=O)(=O)c4ccc5c(c4)CCO5)ccc3c2=O)n[nH]1]

### Step 1

A suspension of 6-bromo-2-((5-methyl-1H-pyrazol-3-yl)methyl)phthalazin-1(2H)-one (intermediate 52, 120 mg, 50% Wt, 1 Eq, 188 μmol), 2,3-dihydrobenzofuran-5-thiol (41 mg, 91% Wt, 1.3 Eq, 0.25 mmol), cesium carbonate (153 mg, 2.5 Eq, 470 μmol), Pd2(dba)3 (8.61 mg, 0.05 Eq, 9.40 μmol) and Xantphos (10.9 mg, 0.10 Eq, 18.8 μmol) in DMF (2 mL) was heated in the microwave at 120° C. for 1.5 h. 2,3-dihydrobenzofuran-5-thiol (41 mg, 91% Wt, 1.3 Eq, 0.25 mmol), Pd2(dba)3 (8.61 mg, 0.05 Eq, 9.40 μmol) and Xantphos (10.9 mg, 0.10 Eq, 18.8 μmol) were added and the mixture was heated in the microwave at 120° C. for 1 h. The reaction mixture was concentrated in vacuo to afford the crude product. The crude product was purified by chromatography on silica gel to afford 6-((2,3-dihydrobenzofuran-5-yl)thio)-2-((5-methyl-1H-pyrazol-3-yl)methyl)phthalazin-1(2H)-one (69 mg) as a clear orange oil. MS (ES⁺): 391 (M+H)⁺

### Step 2

A solution of 6-((2,3-dihydrobenzofuran-5-yl)thio)-2-((5-methyl-1H-pyrazol-3-yl)methyl)phthalazin-1(2H)-one (65 mg, 94% Wt, 1 Eq, 0.16 mmol) in DMF (2 m) was treated with OXONE® (0.24 g, 2.5 Eq, 0.39 mmol) and the reaction mixture was stirred at RT for 20 h. Water (5 mL was added and the mixture was extracted with DCM (3×5 mL). The combined organic extracts were dried (phase separator) and concentrated in vacuo to afford the crude product. The crude product was azeotroped with toluene (3 times). The crude product was purified by chromatography on silica gel to afford the title compound (27.3 mg) as a pale yellow solid after drying in vacuo (45° C., 20 h). ¹H NMR (400 MHz, DMSO-d6) 12.29 (s, 1H), 8.61 (d, J=1.8 Hz, 1H), 8.58 (s, 1H), 8.41 (d, J=8.4 Hz, 1H), 8.26 (dd, =0.1 mm, 1H), 7.86 (t, J=1.7 Hz, 1H), 7.79 (dd, J=8.5, 2.2 Hz, 1H), 6.98 (d, J=8.5 Hz, 1H), 5.83 (s, 1H), 5.21 (s, 2H), 4.64 (t, J=8.8 Hz, 2H), 3.24 (t, J=8.8 Hz, 2H), 2.13 (s, 3H). MS (ES⁺): 423 (M+H)⁺

### Example 210—2-((6-(1-methyl-1H-pyrazol-3-ylsulfonyl)-1-oxophthalazin-2(1H)-yl)methyl)benzamide

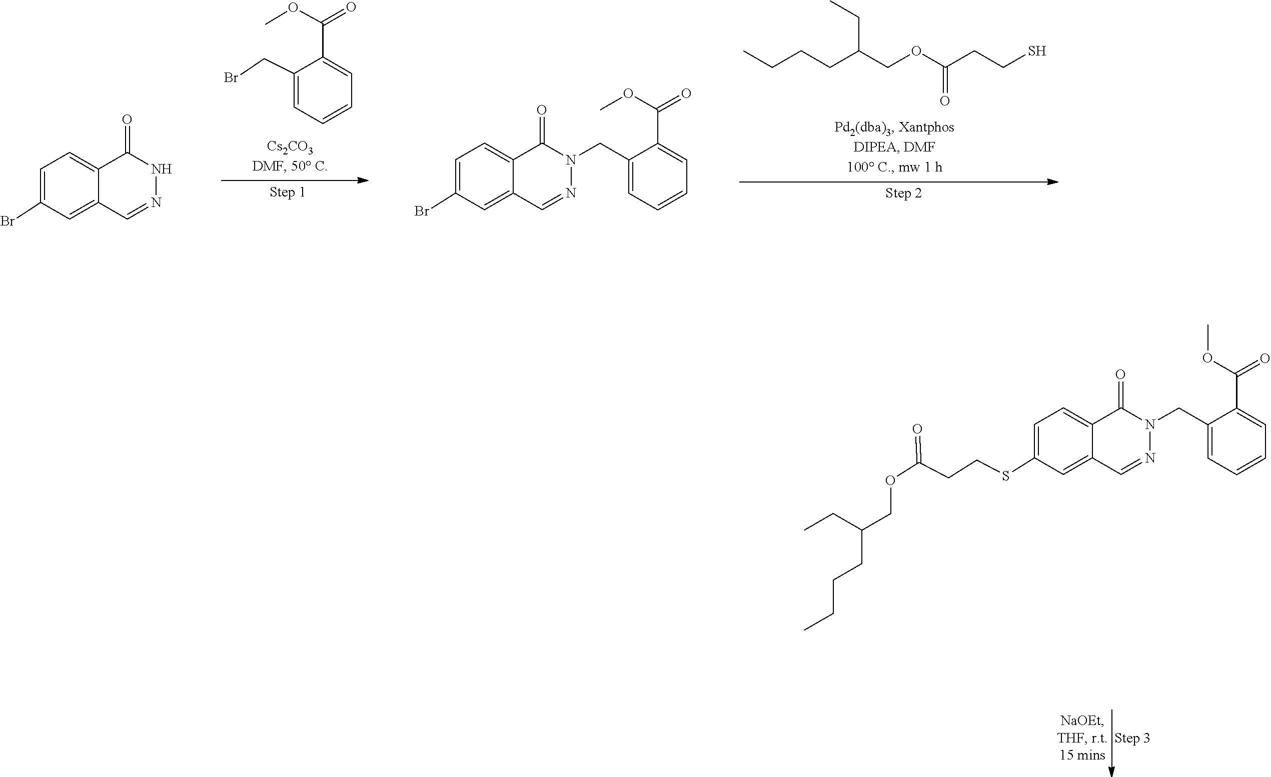 [SMILES: O=c1[nH]ncc2cc(Br)ccc12 ; COC(=O)c1ccccc1CBr ; COC(=O)c1ccccc1Cn1ncc2cc(Br)ccc2c1=O ; CCCCC(CC)COC(=O)CCS ; CCCCC(CC)COC(=O)CCSc1ccc2c(=O)n(Cc3ccccc3C(=O)OC)ncc2c1]

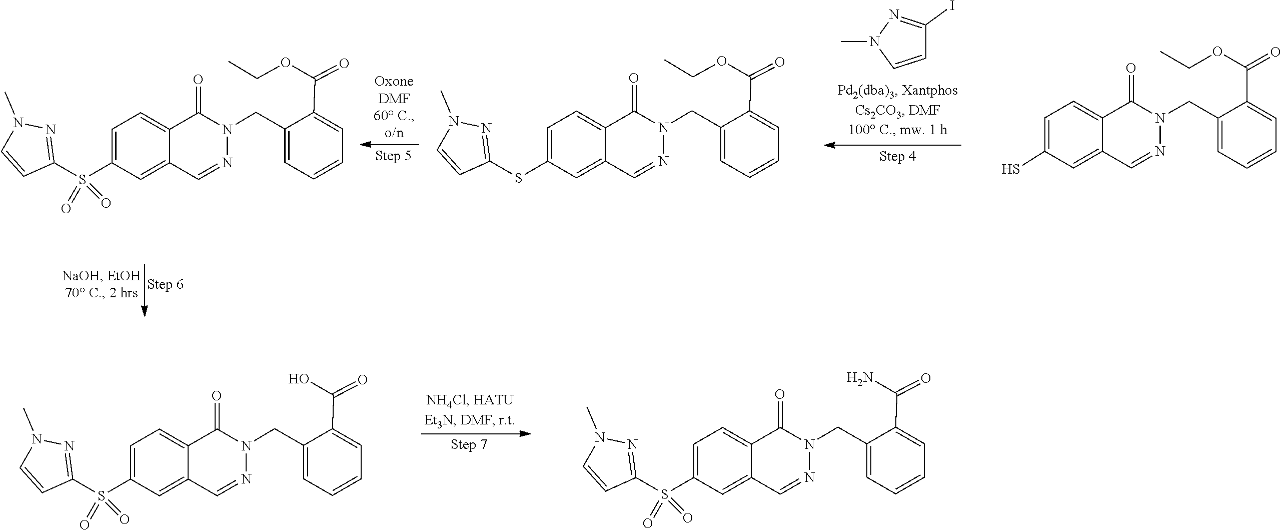

### Step 1

To the solution of methyl 2-(bromomethyl)benzoate (1.0 g, 4.4 mmol), Cs₂CO₃ (4.3 g, 13.2 mmol) in DMF (10 mL) was added 6-bromophthalazin-1(2H)-one (986 mg, 4.4 mmol) at RT, and the reaction mixture was stirred at 50° C. overnight. After LCMS indicated the reaction completed, the reaction mixture was quenched with water (30 mL) and filtered. The cake was dissolved DCM (40 mL), and washed with brine, dried over Na₂SO₄ and filtered. The filtrate was concentrated at 30° C. under reduced pressure, the residue was triturated with hexane and MTBE (10/1), filtered and dried at 30° C. under reduced pressure to give methyl 2-((6-bromo-1-oxophthalazin-2(1H)-yl)methyl)benzoate (1.5 g) as a yellow solid. MS (ES⁺): 373.0 (M+H)⁺

### Step 2

To the solution of methyl 2-((6-bromo-1-oxophthalazin-2(1H)-yl)methyl)benzoate (1.5 g, 4.0 mmol), Pd₂(dba)₃ (366 mg, 0.4 mmol), Xantphos (462 mg, 0.8 mmol), DIPEA (3.9 g, 12 mmol) in DMF (20 mL) was added 2-ethylhexyl 3-mercaptopropanoate (1.3 g, 6 mmol) at RT under N₂, and the reaction mixture was stirred at 100° C. for 1 h under microwave irritation. After LCMS indicated the reaction completed, the reaction mixture was filtered. To the mixture was added EtOAc (10 mL), the combined organic layer was washed by brine, separated and extracted with EtOAc (10 mL×3), dried over Na₂SO₄, filtered and concentrated at 45° C. under reduced pressure, and the residue was purified by flash column chromatography to give methyl 2-((6-(3-(2-ethylhexyloxy)-3-oxopropylthio)-1-oxophthalazin-2(1H)-yl)methyl)benzoate (1.8 g) as a yellow solid. MS (ES⁺): 511.0 (M+H)⁺

### Step 3

A solution of methyl 2-((6-(3-(2-ethylhexyloxy)-3-oxopropylthio)-1-oxophthalazin-2(1H)-yl)methyl)benzoate (1.8 g, 3.5 mmol) in THF (10 mL) was added NaOEt (286 mg, 4.2 mmol) at RT. The reaction mixture was stirred at RT for 15 mins. After LCMS indicated the reaction completed, the reaction mixture was quenched with 0.5 N HCl aqueous to pH=5, separated and extracted with EtOAc (10 mL×2). The combined organic layer was washed with brine, dried over Na₂SO₄, filtered and concentrated at 40° C. under reduced pressure to give ethyl 2-((6-mercapto-1-oxophthalazin-2(1H)-yl)methyl) benzoate (1.1 g) as brown solid. MS (ES⁺): 341.1 (M+H)⁺

### Step 4

To the solution of ethyl 2-((6-mercapto-1-oxophthalazin-2(1H)-yl)methyl) benzoate (250 mg, 0.74 mmol), 3-iodo-1-methyl-1H-pyrazole (154 mg, 0.74 mmol), Pd₂(dba)₃ (64 mg, 0.07 mmol), Xantphos (81 mg, 0.14 mmol), Cs₂CO₃ (722 mg, 2.22 mmol) in DMF (4 mL) at RT, and the reaction mixture was stirred at 100° C. for 1 h under microwave irritation. After LCMS indicated the reaction completed, the reaction mixture was filtered. The mixture was added ethyl acetate (5 mL), the combined organic layer was washed by brine, separated and extracted with ethyl acetate (5 mL×3). dried over Na₂SO₄, filtered and concentrated at 45° C. under reduced pressure, and the residue was purified by flash column chromatography to give ethyl 2-((6-(1-methyl-1H-pyrazol-3-ylthio)-1-oxophthalazin-2(1H)-yl)methyl)benzoate (140 mg) as a yellow solid. MS (ES⁺): 420.8 (M+H)⁺

### Step 5

To a solution of ethyl 2-((6-(1-methyl-1H-pyrazol-3-ylthio)-1-oxophthalazin-2(1H)-yl)methyl)benzoate (140 mg, 0.33 mmol) in DMF (3 mL) was added OXONE® (608 mg, 0.99 mmol) at RT, and the reaction mixture was stirred at 60° C. overnight. After LCMS indicated the reaction completed, the reaction mixture was filtered and extracted with DCM (10 mL×3), the organic layer was concentrated at 30° C. under reduced pressure. The residue was purified by flash column chromatography to give ethyl 2-((6-(1-methyl-1H-pyrazol-3-ylsulfonyl)-1-oxophthalazin-2(1H)-yl)methyl)benzoate (120 mg) as a white solid. MS (ES⁺): 453.0 (M+H)⁺

### Step 6

A mixture of methyl ethyl 2-((6-(1-methyl-1H-pyrazol-3-ylsulfonyl)-1-oxophthalazin-2(1H)-yl)methyl) benzoate (120 mg, 0.26 mmol), NaOH (0.65 mL, 1.3 mmol, 2M) and EtOH (5 mL) was stirred at 70° C. for 2 h. After LCMS indicated the reaction completed, the mixture was quenched with 2 N HCl. The mixture was concentrated at 40° C. to remove organic solvent; the residue was separated and extracted with ethyl acetate (10 mL×3). The separated organics were washed with brine, dried over Na₂SO₄ and filtered. The filtrate was concentrated 40° C. under reduced pressure to give 2-((6-(1-methyl-1H-pyrazol-3-ylsulfonyl)-1-oxophthalazin-2(1H)-yl)methyl)benzoic acid (93 mg) as yellow oil. MS (ES⁺): 424.9 (M+H)⁺

### Step 7

A mixture of 2-((6-(1-methyl-1H-pyrazol-3-ylsulfonyl)-1-oxophthalazin-2(1H)-yl)methyl)benzoic acid (93 mg 0.22 mmol), NH₄Cl (47 mg, 0.88 mmol), HATU (167 mg, 0.44 mmol), and Et₃N (89 mg, 0.88 mmol) in DMF (5 mL) was stirred at RT for 16 h. After LCMS indicated the reaction completed. The residue was purified by prep-HPLC. The fractions were concentrated at 42° C. under reduced pressure to remove MeCN, and the residue was lyophilized to give the title compound (5.78 mg) as white solid. ¹H NMR (400 MHz, DMSO-d6) δ: 8 (s, 1H), 8.67 (d, J=1.6 Hz, 1H), 8.46 (d, J=8.4 Hz, 1H), 8.29 (dd, J=8.4, 1.6 Hz, 1H), 7.98 (d, J=2.0 Hz, 1H), 7.95 (br, 1H), 7.54-7.50 (m, 2H), 7.31 (dd, J=5.6, 3.2 Hz, 2H), 6.99-6.98 (m, 1H), 6.97 (d, J=2.4 Hz, 1H), 5.56 (s, 2H), 3.92 (s, 3H). MS (ES⁺): 424.3 (M+H)⁺

### Example 211—2-((1H-pyrazol-3-yl)methyl)-6-(pyridin-2-ylsulfonyl)phthalazin-1(2H)-one

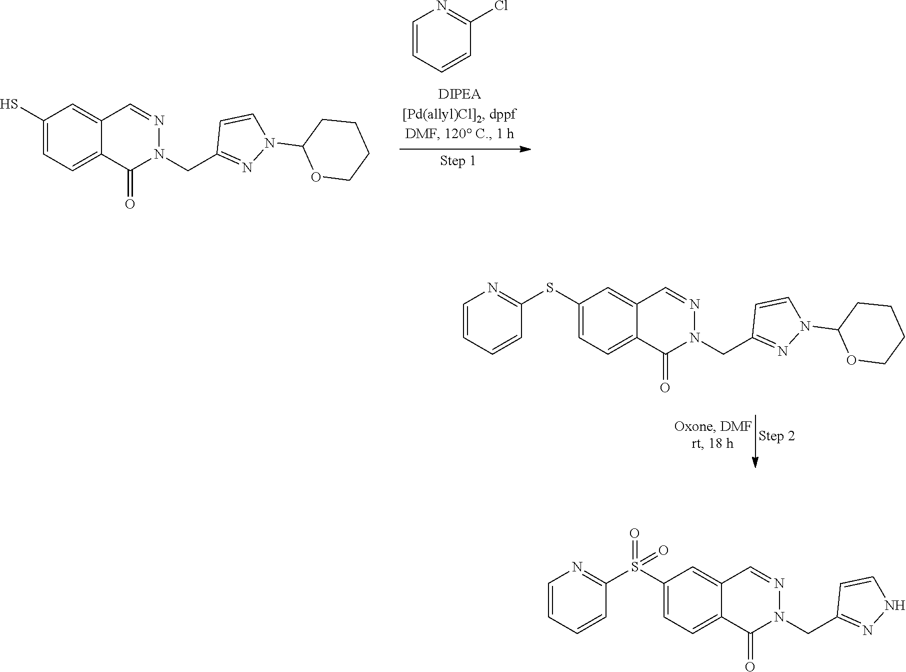 [SMILES: Clc1ccccn1 ; O=c1c2ccc(S)cc2cnn1Cc1ccn(C2CCCCO2)n1 ; O=c1c2ccc(Sc3ccccn3)cc2cnn1Cc1ccn(C2CCCCO2)n1 ; O=c1c2ccc(S(=O)(=O)c3ccccn3)cc2cnn1Cc1cc[nH]n1]

### Step 1

A solution of 6-mercapto-2-((1-(tetrahydro-2H-pyran-2-yl)-1H-pyrazol-3-yl)methyl)phthalazin-1(2H)-one (Intermediate 49, 38 mg, 80% Wt, 1 Eq, 88 μmol), 2-chloropyridine (12 mg, 10 μL, 1.2 Eq, 0.11 mmol) and DIPEA (22 mg, 30 μL, 2.0 Eq, 0.17 mmol) in DMF (1 mL) was sparged with N₂ for 5 minutes, allylpalladium chloride dimer (3.2 mg, 0.10 Eq, 8.8 μmol) and dppf (9.8 mg, 0.20 Eq, 18 μmol) were added and the reaction mixture was stirred at 120° C. for 1 h. The reaction mixture was allowed to cool to RT and then concentrated in vacuo to afford the crude product. The crude product was purified by chromatography on silica gel to afford 6-(pyridin-2-ylthio)-2-((1-(tetrahydro-2H-pyran-2-yl)-1H-pyrazol-3-yl)methyl)phthalazin-1(2H)-one (36 mg) as a yellow solid. MS (ES⁺): 420 (M+H)⁺

### Step 2

A suspension of 6-(pyridin-2-ylthio)-2-((1-(tetrahydro-2H-pyran-2-yl)-1H-pyrazol-3-yl)methyl)phthalazin-1(2H)-one (36 mg, 61% Wt, 1 Eq, 52 μmol) and OXONE® (0.160 g, 5.0 Eq, 260 μmol) in DMF (1 mL) was stirred at RT for 18 h. The reaction mixture was diluted with DCM (5 mL) and washed with water (5 mL). The organic layer was collected and the aqueous was extracted with DCM (2×5 mL). The combined organic extracts were dried (phase separator) and concentrated in vacuo to afford the crude product. The crude product was purified by chromatography on silica gel to afford the title compound (3 mg) as a pale yellow solid. ¹H NMR (400 MHz, DMSO-d6) δ 12.65 (s, 1H), 8.72-8.67 (m, 2H), 8.65 (s, 1H), 8.48 (d, J=8.4 Hz, 1H), 8.34-8.28 (m, 2H), 8.20 (td, J=7.8, 1.8 Hz, 1H), 7.73 (ddd, J=7.7, 4.6, 1.2 Hz, 1H), 7.62 (s, 1H), 6.14 (s, 1H), 5.31 (s, 2H). MS (ES⁺): 368 (M+H)⁺

The following compounds were made using similar procedures described above:

| Example | | |
|---|---|---|
| No. | Example Structure/Name | LCMS/¹H NMR data |
| 212 | 6-((4-(difluoromethoxy)phenyl)sulfonyl)-2-((1-methyl-1H-pyrazol-3- yl)methyl)phthalazin-1(2H)-one 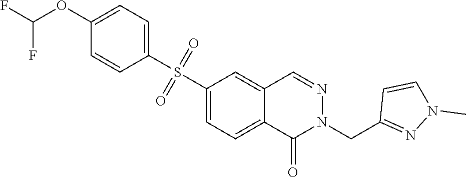 [SMILES: Cn1ccc(Cn2ncc3cc(S(=O)(=O)c4ccc(OC(F)F)cc4)ccc3c2=O)n1] | MS (ES⁺): 447 (M + H)⁺ ¹H NMR (400 MHz, DMSO-d6) δ 8.68 (d, J = 1.8 Hz, 1H), 8.59 (d, J = 0.7 Hz, 1H), 8.43 (d, J = 8.4 Hz, 1H), 8.31 (dd, J = 8.5, 1.9 Hz, 1H), 8.14-8.04 (m, 2H), 7.61-7.21 (m, 4H), 6.09 (d, J = 2.2 Hz, 1H), 5.25 (s, 2H), 3.75 (s, 3H). |
| 213 | 2-((1-methyl-1H-pyrazol-3-yl)methyl)-6-((2-methyl-2,3- dihydrobenzofuran-5-yl)sulfonyl)phthalazin-1(2H)-one 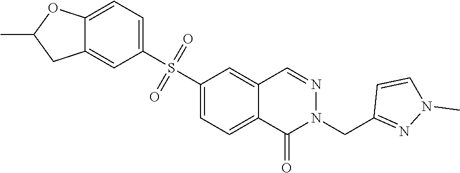 | MS (ES⁺): 437 (M + H)⁺ ¹H NMR (DMSO-d6) δ: 8.61 (d, J = 1.8 Hz, 1H), 8.58 (s, 1H), 8.41 (d, J = 8.4 Hz, 1H), 8.26 (dd, J = 8.4, 1.9 Hz, 1H), 7.89-7.75 (m, 2H), 7.56 (d, J = 2.2 Hz, 1H), 6.95 (d, J = 8.5 Hz, 1H), 6.09 (d, J = 2.2 Hz, 1H), 5.25 (s, 2H), 5.11-4.98 (m, 1H), 3.75 (s, 3H), 3.38 (dd, J = 16.3, 9.0 Hz, 1H), 2.84 (dd, J = 16.3, 7.6 Hz, 1H), 1.37 (d, J = 6.2 Hz, 3H). |
| 214 | 2-((1-methyl-1H-pyrazol-3-yl)methyl)-6-((2-methylthiazol-4- yl)sulfonyl)phthalazin-1(2H)-one 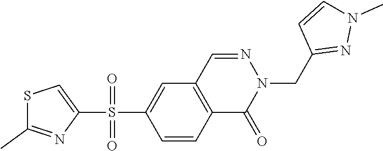 | MS (ES⁺): 402 (M + H)⁺ ¹H NMR (DMSO-d6) δ: 8.69 (s, 1H), 8.66 (d, J = 1.8 Hz, 1H), 8.65 (s, 1H), 8.48 (d, J = 8.4 Hz, 1H), 8.30 (dd, J = 8.4, 1.9 Hz, 1H), 7.57 (d, J = 2.2 Hz, 1H), 6.11 (d, J = 2.3 Hz, 1H), 5.26 (s, 2H), 3.75 (s, 3H), 2.64 (s, 3H). |
| 215 | 6-((2,3-dihydrobenzo[b][1,4]dioxin-6-yl)sulfonyl)-2-((1-methyl-1H- pyrazol-3-yl)methyl)phthalazin-1(2H)-one 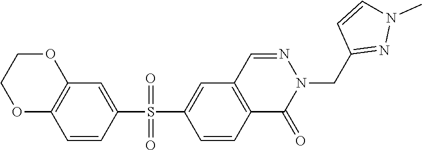 [SMILES: Cn1ccc(Cn2ncc3cc(S(=O)(=O)c4ccc5c(c4)OCCO5)ccc3c2=O)n1] | MS (ES⁺): 439 (M + H)⁺ ¹H NMR (DMSO-d6) δ: 8.64 (d, J = 1.9 Hz, 1H), 8.58 (s, 1H), 8.41 (d, J = 8.4 Hz, 1H), 8.28 (dd, J = 8.4, 1.9 Hz, 1H), 7.56 (d, J = 2.2 Hz, 1H), 7.51-7.46 (m, 2H), 7.10 (d, J = 9.1 Hz, 1H), 6.09 (d, J = 2.2 Hz, 1H), 5.25 (s, 2H), 4.37-4.24 (m, 4H), 3.75 (s, 3H). |
| 216 | 6-((4-ethoxyphenyl)sulfonyl)-2-((1-methyl-1H-pyrazol-3- yl)methyl)phthalazin-1(2H)-one 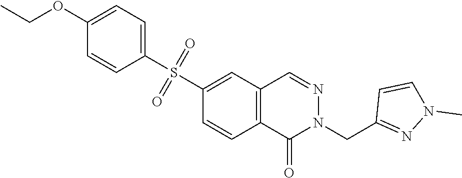 [SMILES: CCOc1ccc(S(=O)(=O)c2ccc3c(=O)n(Cc4ccn(C)n4)ncc3c2)cc1] | MS (ES⁺): 425 (M + H)⁺ ¹H NMR (DMSO-d6) δ: 8.62 (d, J = 1.8 Hz, 1H), 8.58 (s, 1H), 8.41 (d, J = 8.3 Hz, 1H), 8.26 (dd, J = 8.5, 1.9 Hz, 1H), 7.96-7.90 (m, 2H), 7.56 (d, J = 2.2 Hz, 1H), 7.19-7.13 (m, 2H), 6.09 (d, J = 2.1 Hz, 1H), 5.24 (s, 2H), 4.11 (q, J = 7.0, 7.0, 7.0 Hz, 2H), 3.75 (s, 3H), 1.32 (t, J = 7.0, 7.0 Hz, 3H). |
| 217 | (R)-2-((1-methyl-1H-pyrazol-3-yl)methyl)-6-((2-methyl-2,3- dihydrobenzofuran-5-yl)sulfonyl)phthalazin-1(2H)-one 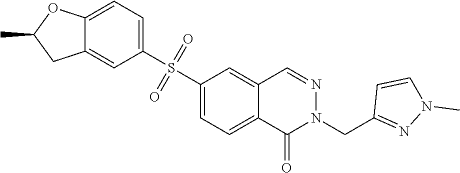 | MS (ES⁺): 437 (M + H)⁺ ¹H NMR (DMSO-d6) δ: 8.61 (d, J = 1.8 Hz, 1H), 8.58 (s, 1H), 8.41 (d, J = 8.4 Hz, 1H), 8.26 (dd, J = 8.4, 1.9 Hz, 1H), 7.89-7.75 (m, 2H), 7.56 (d, J = 2.2 Hz, 1H), 6.95 (d, J = 8.5 Hz, 1H), 6.09 (d, J = 2.2 Hz, 1H), 5.25 (s, 2H), 5.11-4.98 (m, 1H), 3.75 (s, 3H), 3.38 (dd, J = 16.3, 9.0 Hz, 1H), 2.84 (dd, J = 16.3, 7.6 Hz, 1H), 1.37 (d, J = 6.2 Hz, 3H). RT: 3.496 min, 100% ee |
| 218 | 20((1-methyl-1H-pyrazol-3-yl)methyl)-6-((4- (trifluoromethoxy)phenyl)sulfonyl)phthalazin-1(2H)-one 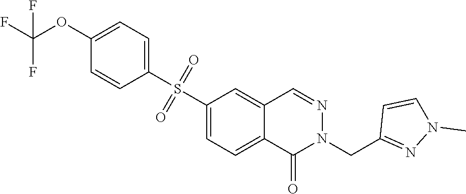 [SMILES: Cn1ccc(Cn2ncc3cc(S(=O)(=O)c4ccc(OC(F)(F)F)cc4)ccc3c2=O)n1] | MS (ES⁺): 465 (M + H)⁺ ¹H NMR (400 MHz, DMSO-d6) δ 8.71 (d, J = 1.8 Hz, 1H), 8.59 (s, 1H), 8.44 (d, J = 8.4 Hz, 1H), 8.33 (dd, J = 8.4, 1.9 Hz, 1H), 8.23- 8.11 (m, 2H), 7.65 (d, J = 8.5 Hz, 2H), 7.56 (d, J = 2.2 Hz, 1H), 6.09 (d, J = 2.2 Hz, 1H), 5.25 (s, 2H), 3.75 (s, 3H). |
| 219 | (S)-2-((1-methyl-1H-pyrazol-3-yl)methyl)-6-((2-methyl-2,3- dihydrobenzofuran-5-yl)sulfonyl)phthalazin-1(2H)-one 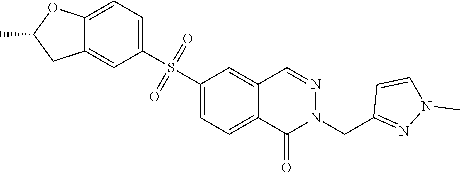 | MS (ES⁺): 437 (M + H)⁺ ¹H NMR (DMSO-d6) δ: 8.61 (d, J = 1.8 Hz, 1H), 8.58 (s, 1H), 8.41 (d, J = 8.4 Hz, 1H), 8.26 (dd, J = 8.4, 1.9 Hz, 1H), 7.89-7.75 (m, 2H), 7.56 (d, J = 2.2 Hz, 1H), 6.95 (d, J = 8.5 Hz, 1H), 6.09 (d, J = 2.2 Hz, 1H), 5.25 (s, 2H), 5.11-4.98 (m, 1H), 3.75 (s, 3H), 3.38 (dd, J = 16.3, 9.0 Hz, 1H), 2.84 (dd, J = 16.3, 7.6 Hz, 1H), 1.37 (d, J = 6.2 Hz, 3H). RT: 3.953 min, 100% ee |
| 220 | 2-((1H-pyrazol-3-yl)methyl)-6-((3-methylbenzofuran-5- yl)sulfonyl)phthalazin-1(2H)-one 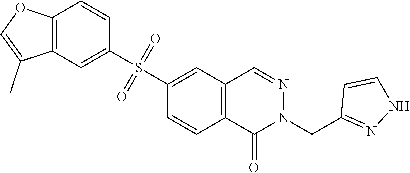 [SMILES: Cc1coc2ccc(S(=O)(=O)c3ccc4c(=O)n(Cc5cc[nH]n5)ncc4c3)cc12] | MS (ES⁺): 421 (M + H)⁺ ¹H NMR (DMSO-d6) δ: 12.64 (s, 1H), 8.70 (d, J = 1.8 Hz, 1H), 8.59 (s, 1H), 8.42 (d, J = 8.3 Hz, 1H), 8.38-8.31 (m, 2H), 8.00 (d, J = 1.5 Hz, 1H), 7.94 (dd, J = 8.7, 2.0 Hz, 1H), 7.81 (d, J = 8.7 Hz, 1H), 7.61 (s, 1H), 6.11 (s, 1H), 5.29 (s, 2H), 2.27 (d, J = 1.3 Hz, 3H). |
| 221 | 2-((1H-pyrazol-3-yl)methyl)-6-((3,4-dihydro-2H- benzo[b][1,4]oxazin-6-yl)sulfonyl)phthalazin-1(2H)-one 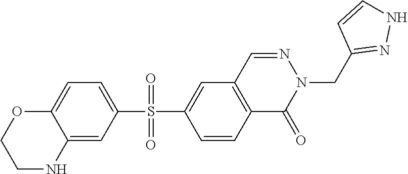 [SMILES: O=c1c2ccc(S(=O)(=O)c3ccc4c(c3)NCCO4)cc2cnn1Cc1cc[nH]n1] | MS (ES⁺): 424 (M + H)⁺ ¹H NMR (400 MHz, DMSO-d6) δ 12.65 (s, 1H), 8.62 (d, J = 9.0 Hz, 1H), 8.55 (d, J = 1.8 Hz, 1H), 8.43 (d, J = 8.4 Hz, 1H), 8.19 (dd, J = 8.5, 1.8 Hz, 1H), 7.62 (s, 1H), 7.14 (d, J = 2.3 Hz, 1H), 7.09 (dd, J = 8.4, 2.3 Hz, 1H), 6.84 (d, J = 8.4 Hz, 1H), 6.39 (s, 1H), 6.12 (d, J = 2.4 Hz, 1H), 5.29 (s, 2H), 4.16 (t, J = 4.4 Hz, 2H), 3.28 (d, J = 5.8 Hz, 2H) |
| 222 | 2-((1H-pyrazol-3-yl)methyl)-6-(benzofuran-6- ylsulfonyl)phthalazin-1(2H)-one 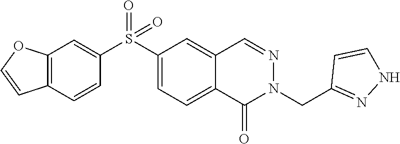 [SMILES: O=c1c2ccc(S(=O)(=O)c3ccc4ccoc4c3)cc2cnn1Cc1cc[nH]n1] | MS (ES⁺): 408 (M + H)⁺ ¹H NMR (DMSO-d6) δ: 12.64 (s, 1H), 8.70 (d, J = 1.8 Hz, 1H), 8.58 (s, 1H), 8.42 (d, J = 8.4 Hz, 1H), 8.38-8.32 (m, 2H), 8.31 (d, J = 2.2 Hz, 1H), 7.93 (d, J = 8.2 Hz, 1H), 7.87 (dd, J = 8.3, 1.6 Hz, 1H), 7.64-7.56 (m, 1H), 7.14 (dd, J = 2.2, 1.0 Hz, 1H), 6.11 (s, 1H), 5.29 (s, 2H) |
| 223 | 2-((1H-pyrazol-3-yl)methyl)-6-(furo[3,2-b]pyridin-5- ylsulfonyl)phthalazin-1(2H)-one 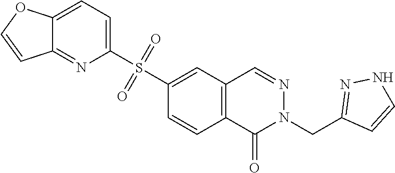 [SMILES: O=c1c2ccc(S(=O)(=O)c3ccc4occc4n3)cc2cnn1Cc1cc[nH]n1] | MS (ES⁺): 408 (M + H)⁺ ¹H NMR (DMSO-d6) δ: 12.64 (s, 1H), 8.70 (d, J = 1.8 Hz, 1H), 8.64 (s, 1H), 8.57 (d, J = 2.4 Hz, 1H), 8.47 (d, J = 8.4 Hz, 1H), 8.42 (dd, J = 8.6, 1.0 Hz, 1H), 8.34 (dd, J = 8.4, 1.8 Hz, 1H), 8.29 (d, J = 8.6 Hz, 1H), 7.62 (s, 1H), 7.27 (dd, J = 2.4, 1.0 Hz, 1H), 6.13 (s, 1H), 5.30 (s, 2H). |
| 224 | 2-((1H-pyrazol-3-yl)methyl)-6-((2-methylbenzofuran-5- yl)sulfonyl)phthalazin-1(2H)-one 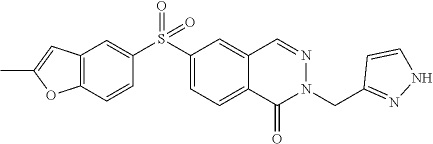 [SMILES: Cc1cc2cc(S(=O)(=O)c3ccc4c(=O)n(Cc5cc[nH]n5)ncc4c3)ccc2o1] | MS (ES⁺): 421 (M + H)⁺ ¹H NMR (DMSO-d6) δ: 12.64 (s, 1H), 8.66 (d, J = 1.8 Hz, 1H), 8.59 (s, 1H), 8.42 (d, J = 8.5 Hz, 1H), 8.30 (dd, J = 8.5, 1.9 Hz, 1H), 8.28 (d, J = 1.9 Hz, 1H), 7.86 (dd, J = 8.6, 2.0 Hz, 1H), 7.75 (d, J = 8.7 Hz, 1H), 7.61 (s, 1H), 6.81-6.70 (m, 1H), 6.11 (s, 1H), 5.29 (s, 2H), 2.48 (d, J = 1.1 Hz, 3H). |
| 225 | 2-((1H-pyrazol-3-yl)methyl)-6-(furo[2,3-b]pyridin-5- ylsulfonyl)phthalazin-1(2H)-one 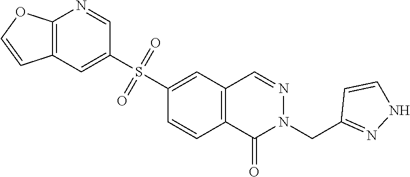 [SMILES: O=c1c2ccc(S(=O)(=O)c3cnc4occc4c3)cc2cnn1Cc1cc[nH]n1] | MS (ES⁺): 408 (M + H)⁺ ¹H NMR (400 MHz, DMSO-d6) δ 12.65 (s, 1H), 8.98 (d, J = 2.3 Hz, 1H), 8.83 (d, J = 2.4 Hz, 1H), 8.74 (d, J = 1.7 Hz, 1H), 8.59 (s, 1H), 8.48-8.37 (m, 2H), 8.34 (d, J = 2.5 Hz, 1H), 7.57 (s, 1H), 7.19 (d, J = 2.5 Hz, 1H), 6.11 (d, J = 2.2 Hz, 1H), 5.30 (s, 2H). |
| 226 | 6-((1H-indol-5-yl)sulfonyl)-2-((1H-pyrazol-3-yl)methyl)phthalazin- 1(2H)-one 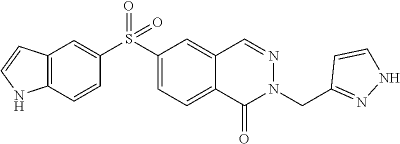 [SMILES: O=c1c2ccc(S(=O)(=O)c3ccc4[nH]ccc4c3)cc2cnn1Cc1cc[nH]n1] | MS (ES⁺): 409 (M + H)⁺ ¹H NMR (DMSO-d6) δ: 12.63 (s, 1H), 11.72 (s, 1H), 8.61 (d, J = 12.8 Hz, 2H), 8.40 (d, J = 8.4 Hz, 1H), 8.31 (s, 1H), 8.27 (d, J = 8.5 Hz, 1H), 7.66 (d, J = 8.7 Hz, 1H), 7.63-7.54 (m, 3H), 6.67 (s, 1H), 6.11 (s, 1H), 5.29 (s, 2H). |
| 227 | 2-((1H-pyrazol-3-yl)methyl)-6-(benzo[b]thiophen-5- ylsulfonyl)phthalazin-1(2H)-one 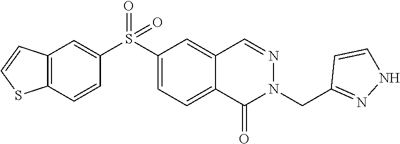 [SMILES: O=c1c2ccc(S(=O)(=O)c3ccc4sccc4c3)cc2cnn1Cc1cc[nH]n1] | MS (ES⁺): 423 (M + H)⁺ ¹H NMR (DMSO-d6) δ: 12.64 (s, 1H), 8.69 (d, J = 1.8 Hz, 1H), 8.63 (d, J = 1.9 Hz, 1H), 8.60 (s, 1H), 8.43 (d, J = 8.5 Hz, 1H), 8.36- 8.28 (m, 2H), 8.02 (d, J = 5.5 Hz, 1H), 7.90 (dd, J = 8.6, 1.9 Hz, 1H), 7.69 (d, J = 5.5, 0.8 Hz, 1H), 7.60 (s, 1H), 6.11 (s, 1H), 5.29 (s, 2H). |
| 228 | 2-((1H-pyrazol-3-yl)methyl)-6-((1-cyclopropyl-1H-pyrazol-4- yl)sulfonyl)phthalazin-1(2H)-one 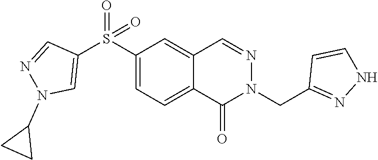 [SMILES: O=c1c2ccc(S(=O)(=O)c3cnn(C4CC4)c3)cc2cnn1Cc1cc[nH]n1] | MS (ES⁺): 397 (M + H)⁺ ¹H NMR (DMSO-d6) δ: 12.65 (s, 1H), 8.67 (s, 1H), 8.62-8.55 (m, 2H), 8.44 (d, J = 8.5 Hz, 1H), 8.30 (dd, J = 8.5, 1.9 Hz, 1H), 8.00 (d, J = 0.8 Hz, 1H), 7.62 (s, 1H), 6.13 (s, 1H), 5.31 (s, 2H), 3.83 (tt, J = 7.4, 7.4, 3.8, 3.8 Hz, 1H), 1.13-1.06 (m, 2H), 1.05-0.93 (m, 2H). |
| 229 | 2-((1H-pyrazol-3-yl)methyl)-6-((2,3-dihydro-[1,4]dioxino[2,3- b]pyridin-6-yl)sulfonyl)phthalazin-1(2H)-one 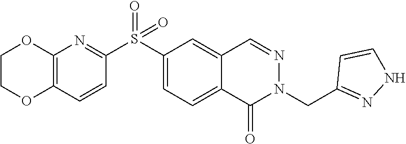 [SMILES: O=c1c2ccc(S(=O)(=O)c3ccc4c(n3)OCCO4)cc2cnn1Cc1cc[nH]n1] | MS (ES⁺): 426 (M + H)⁺ ¹H NMR (400 MHz, DMSO-d6) δ 12.65 (s, 1H), 8.65 (s, 1H), 8.62 (d, J = 1.8 Hz, 1H), 8.46 (d, J = 8.4 Hz, 1H), 8.26 (dd, J = 8.4, 1.8 Hz, 1H), 7.84 (d, J = 8.1 Hz, 1H), 7.62 (s, 1H), 7.56 (d, J = 8.1 Hz, 1H), 6.13 (s, 1H), 5.33- 5.29 (m, 2H), 4.47-4.40 (m, 2H), 4.36-4.29 (m, 2H). |
| 230 | 2-((1H-pyrazol-3-yl)methyl)-6-((2,3-dihydro-[1,4]dioxino[2,3- c]pyridin-7-yl)sulfonyl)phthalazin-1(2H)-one 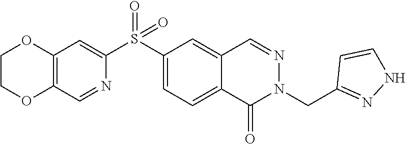 [SMILES: O=c1c2ccc(S(=O)(=O)c3cc4c(cn3)OCCO4)cc2cnn1Cc1cc[nH]n1] | MS (ES⁺): 426 (M + H)⁺ ¹H NMR (DMSO-d6) δ: 12.65 (s, 1H), 8.64 (d, J = 1.8 Hz, 1H), 8.63 (s, 1H), 8.45 (d, J = 8.4 Hz, 1H), 8.28 (dd, J = 8.4, 1.9 Hz, 1H), 8.24 (s, 1H), 7.78 (s, 1H), 7.62 (s, 1H), 6.13 (s, 1H), 5.30 (s, 2H), 4.47-4.36 (m, 4H). |
| 231 | 2-((1H-pyrazol-3-yl)methyl)-6-((1-methyl-1H-indol-5- yl)sulfonyl)phthalazin-1(2H)-one 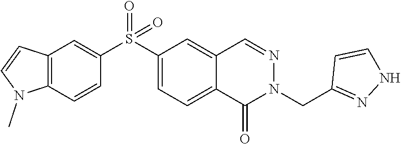 [SMILES: Cn1ccc2cc(S(=O)(=O)c3ccc4c(=O)n(Cc5cc[nH]n5)ncc4c3)ccc21] | MS (ES⁺): 420 (M + H)⁺ ¹H NMR (DMSO-d6) δ: 12.64 (s, 1H), 8.63 (d, J = 1.8 Hz, 1H), 8.59 (s, 1H), 8.39 (d, J = 8.5 Hz, 1H), 8.31 (d, J = 1.8 Hz, 1H), 8.27 (dd, J = 8.5, 1.9 Hz, 1H), 7.72 (dd, J = 8.7, 1.8 Hz, 1H), 7.68 (d, J = 8.8 Hz, 1H), 7.60 (s, 1H), 7.57 (d, J = 3.2 Hz, 1H), 6.68 (d, J = 3.1 Hz, 1H), 6.11 (s, 1H), 5.29 (s, 2H), 3.83 (d, J = 2.0 Hz, 3H). |
| 232 | 6-((1H-pyrazol-4-yl)sulfonyl)-2-((1-cyclopropyl-1H-pyrazol-3- yl)methyl)phthalazin-1(2H)-one 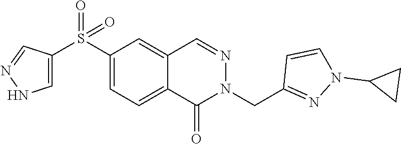 [SMILES: O=c1c2ccc(S(=O)(=O)c3cn[nH]c3)cc2cnn1Cc1ccn(C2CC2)n1] | MS (ES⁺): 397 (M + H)⁺ ¹H NMR (400 MHz, DMSO-d6) δ 13.90 (br s, 1H), 8.63-8.56 (m, 2H), 8.43 (d, J = 8.4 Hz, 1H), 8.37-8.24 (m, 3H), 7.64 (d, J = 2.3 Hz, 1H), 6.07 (d, J = 2.3 Hz, 1H), 5.24 (s, 2H), 3.62 (tt, J = 7.4, 3.6 Hz, 1H), 0.99-0.93 (m, 2H), 0.93-0.87 (m, 2H). |
| 233 | 2-((1H-pyrazol-3-yl)methyl)-6-(pyridin-3-ylsulfonyl) phthalazin-1(2H)-one 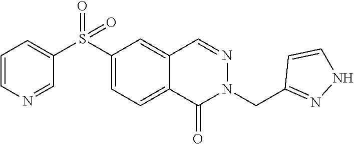 [SMILES: O=c1c2ccc(S(=O)(=O)c3cccnc3)cc2cnn1Cc1cc[nH]n1] | MS (ES⁺): 368 (M + H)⁺ ¹H NMR (DMSO-d6) δ: 12.65 (s, 1H), 9.21 (s, 1H), 8.90 (d, J = 4.8 Hz, 1H), 8.75 (s, 1H), 8.59 (s, 1H), 8.49-8.35 (m, 3H), 7.70 (dd, J = 8.3, 5.0 Hz, 1H), 7.62 (s, 1H), 6.12 (s, 1H), 5.30 (s, 2H). |
| 234 | 2-((1H-pyrazol-3-yl)methyl)-6-((2,3-dihydro-[1,4]dioxino[2,3- b]pyridin-7-yl)sulfonyl)phthalazin-1(2H)-one 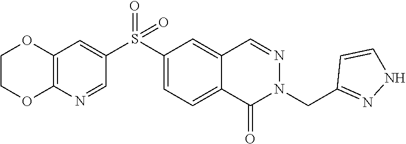 [SMILES: O=c1c2ccc(S(=O)(=O)c3cnc4c(c3)OCCO4)cc2cnn1Cc1cc[nH]n1] | MS (ES⁺): 426 (M + H)⁺ ¹H NMR (DMSO-d6) δ: 12.65 (s, 1H), 8.70 (d, J = 1.8 Hz, 1H), 8.57 (s, 1H), 8.43 (d, J = 8.4 Hz, 1H), 8.39 (d, J = 2.3 Hz, 1H), 8.37 (dd, J = 8.4, 1.8 Hz, 1H), 7.84 (d, J = 2.2 Hz, 1H), 7.62 (s, 1H), 6.12 (s, 1H), 5.30 (s, 2H), 4.53-4.47 (m, 2H), 4.34-4.28 (m, 2H). |
| 235 | 2-((1H-pyrazol-3-yl)methyl)-6-((6-methoxypyridin-3- yl)sulfonyl)phthalazin-1(2H)-one 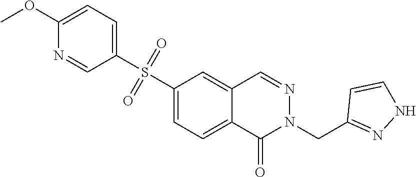 [SMILES: COc1ccc(S(=O)(=O)c2ccc3c(=O)n(Cc4cc[nH]n4)ncc3c2)cn1] | MS (ES⁺): 398 (M + H)⁺ ¹H NMR (400 MHz, DMSO-d6) δ 12.65 (s, 1H), 8.86 (d, J = 2.6 Hz, 1H), 8.69 (d, J = 1.9 Hz, 1H), 8.58 (s, 1H), 8.43 (d, J = 8.4 Hz, 1H), 8.35 (dd, J = 8.4, 1.9 Hz, 1H), 8.23 (dd, J = 8.9, 2.6 Hz, 1H), 7.62 (s, 1H), 7.04 (d, J = 9.0 Hz, 1H), 6.12 (d, J = 2.2 Hz, 1H), 5.30 (s, 2H), 3.94 (s, 3H). |
| 236 | 2-((1-cyclopropyl-1H-pyrazol-3-yl)methyl)-6-((1-methyl-1H- pyrazol-4-yl)sulfonyl)phthalazin-1(2H)-one 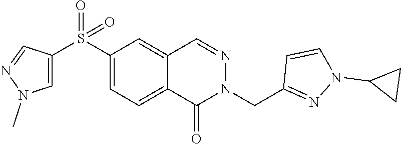 [SMILES: Cn1cc(S(=O)(=O)c2ccc3c(=O)n(Cc4ccn(C5CC5)n4)ncc3c2)cn1] | MS (ES⁺): 411 (M + H)⁺ ¹H NMR (400 MHz, DMSO-d6) δ 8.59 (d, J = 2.9 Hz, 2H), 8.55 (s, 1H), 8.43 (d, J = 8.5 Hz, 1H), 8.29 (dd, J = 8.4, 1.8 Hz, 1H), 8.01 (s, 1H), 7.65 (d, J = 2.3 Hz, 1H), 6.07 (d, J = 2.4 Hz, 1H), 5.24 (d, J = 3.4 Hz, 2H), 3.87 (s, 3H), 3.62 (td, J = 7.3, 3.6 Hz, 1H), 1.01-0.85 (m, 4H). |

### Example 237—6-(1-(2-hydroxyethyl)-1H-pyrazol-3-ylsulfonyl)-2-((2-hydroxypyridin-3-yl)methyl)phthalazin-1(2H)-one

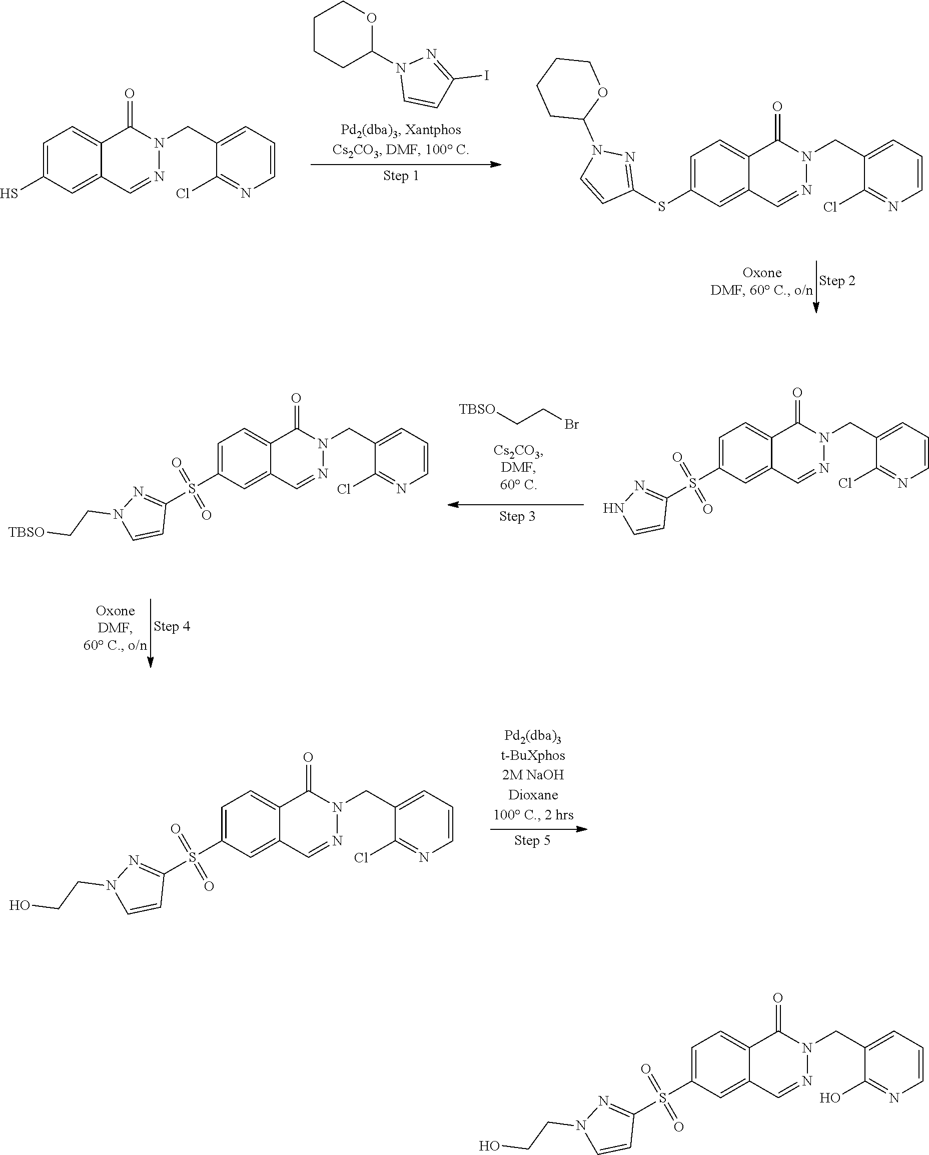

### Step 1

To a mixture of 2-((2-chloropyridin-3-yl)methyl)-6-mercaptophthalazin-1(2H)-one (Intermediate 46, 270 mg, 0.9 mmol), Pd₂(dba)₃ (82 mg, 0.09 mmol), Xantphos (104 mg, 0.18 mmol), Cs₂CO₃ (586.8 mg, 1.8 mmol) in DMF (5 mL) was added 3-iodo-1-(tetrahydro-2H-pyran-2-yl)-1H-pyrazole (Intermediate 54, 278 mg, 1 mmol) at RT, and the reaction mixture was stirred at 100° C. for 1 hour under N₂. After LCMS indicated the reaction was complete, the reaction mixture was filtered through Celite and washed with EtOAc. The filtrate was diluted with water (10 mL) and extracted with EtOAc (10 mL×3). The combined organic layer was washed by brine, dried over Na₂SO₄, filtered and concentrated at 45° C. under reduced pressure. The residue was purified by flash column chromatography to give 2-((2-chloropyridin-3-yl)methyl)-6-(1-(tetrahydro-2H-pyran-2-yl)-1H-pyrazol-3-ylthio)phthalazin-1(2H)-one (290 mg as a yellow solid. MS (ES⁺): 370.0 (M+H)⁺

### Step 2

To a solution of 2-((2-chloropyridin-3-yl)methyl)-6-(1-(tetrahydro-2H-pyran-2-yl)-1H-pyrazol-3-ylthio)phthalazin-1(2H)-one (290 mg, 0.6 mmol) in DMF (3 mL) was added OXONE® (2.2 g, 3.6 mmol) at RT, and the reaction mixture was stirred at 60° C. overnight. After LCMS indicated the reaction was complete, the reaction mixture was filtered through Celite and washed with EtOAc. The filtrate was diluted with water (10 mL) and extracted with EtOAc (5 mL×3). The combined organic layer was washed by brine, dried over Na₂SO₄, filtered and concentrated at 45° C. under reduced pressure. The residue was purified by flash column chromatography to give 6-(1H-pyrazol-3-ylsulfonyl)-2-((2-chloropyridin-3-yl)methyl)phthalazin-1(2H)-one (130 mg) as a white solid. MS (ES⁺): 401.8 (M+H)⁺

### Step 3

To the solution of 6-(1H-pyrazol-3-ylsulfonyl)-2-((2-chloropyridin-3-yl)methyl)phthalazin-1(2H)-one (130 mg, 0.32 mmol) and K₂CO₃ (90 mg, 0.65 mmol) in DMF (3 mL) was added tert-butyl(2-(2-iodophenoxy)ethoxy)dimethylsilane (118 mg, 0.32 mmol) at RT, and the reaction mixture was stirred at 60° C. for 3 h. After LCMS indicated the reaction was complete, the reaction mixture was diluted with water (10 mL) and filtered. The filtered solid was dissolved in DCM (10 mL), washed by water and brine, dried over Na₂SO₄, filtered and concentrated at 30° C. under reduced pressure. The residue was purified by flash column chromatography to give 6-(1-(2-(tert-butyldimethylsilyloxy)ethyl)-1H-pyrazol-3-ylsulfonyl)-2-((2-chloropyridin-3-yl)methyl)phthalazin-1(2H)-one (120 mg) as a yellow solid. ¹H NMR (400 MHz, DMSO-d6) δ: 8.70 (s, 1H), 8.67 (d, J=1.6 Hz, 1H), 8.47 (d, J=8.4 Hz, 1H), 8.36 (dd, J=4.4, 2.0 Hz 1H), 8.31 (dd, J=8.4, 2.0 Hz 1H), 7.98 (d, J=2.4 Hz, 1H), 7.66 (dd, J=7.6, 1.6 Hz, 1H), 7.36 (dd, J=7.6, 4.8 Hz, 1H), 6.97 (d, J=2.4 Hz, 1H), 5.42 (s, 2H), 4.27 (t, J=4.4 Hz, 2H), 3.85 (t, J=5.2 Hz, 2H), 0.57 (s, 9H), −0.32 (t, J=2.8 Hz, 6H). MS (ES⁺): 559.8 (M+H)⁺

### Step 4

To a solution of 6-(1-(2-(tert-butyldimethylsilyloxy)ethyl)-1H-pyrazol-3-ylsulfonyl)-2-((2-chloropyridin-3-yl)methyl)phthalazin-1(2H)-one (120 mg, 0.2 mmol) in DMF (3 mL) was added OXONE® (737 mg, 1.2 mmol) at RT, and the reaction mixture was stirred at 60° C. overnight. After LCMS indicated the reaction was complete, the reaction mixture was filtered through Celite and washed with EtOAc. The filtrate was diluted with water (10 mL) and extracted with EtOAc (5 mL×3). The combined organic layer was washed by brine, dried over Na₂SO₄, filtered and concentrated at 45° C. under reduced pressure. The residue was purified by flash column chromatography to give 2-((2-chloropyridin-3-yl)methyl)-6-(1-(2-hydroxyethyl)-1H-pyrazol-3-ylsulfonyl)phthalazin-1(2H)-one (80 mg) as a white solid. MS (ES⁺): 445.8 (M+H)⁺

### Step 5

To a mixture of 2-((2-chloropyridin-3-yl)methyl)-6-(1-(2-hydroxyethyl)-1H-pyrazol-3-ylsulfonyl)phthalazin-1(2H)-one (80 mg 0.18 mmol), Pd₂(dba)₃ (16.5 mg, 0.018 mmol), t-BuXphos (15.3 mg, 0.036 mmol) in Dioxane (3 mL) was added NaOH (1 mL, 2 mmol, 2M) at RT, and the reaction mixture was stirred at 100° C. for 2 h under N₂. After LCMS indicated the reaction is completed, the reaction mixture was filtered through Celite and washed with EtOAc. The filtrate was diluted with water (10 mL) and extracted with EtOAc (10 mL×3). The combined organic layer was washed by brine, dried over Na₂SO₄, filtered and concentrated at 45° C. under reduced pressure. The residue was purified by prep-HPLC. The relevant fractions were concentrated at 40° C. under reduced pressure to remove MeCN, and the residue was lyophilized to afford the title compound (11.68 mg) as a white solid. ¹H NMR (400 MHz, DMSO-d6) δ: 11.72 (br, 1H), 8.67 (s, 1H), 8.65 (d, J=1.6 Hz, 1H), 8.46 (d, J=8.4 Hz, 1H), 8.30 (dd, J=8.4, 2.0 Hz 1H), 7.98 (d, J=2.4 Hz, 1H), 7.31 (dd, J=6.4, 1.6 Hz, 1H), 7.04 (dd, J=6.8, 1.6 Hz, 1H), 6.96 (d, J=2.0 Hz, 1H), 6.08 (t, J=6.8 Hz, 1H), 5.08 (s, 2H), 4.96 (t, J=5.2 Hz, 1H), 4.22 (t, J=5.2 Hz, 2H), 3.71 (q, J=10.4, 5.2 Hz, 2H). MS (ES⁺): 428.2 (M+H)⁺

### Example 238—6-((1H-pyrazol-4-yl)sulfonyl)-2-((6-methylpyridin-2-yl)methyl)phthalazin-1(2H)-one

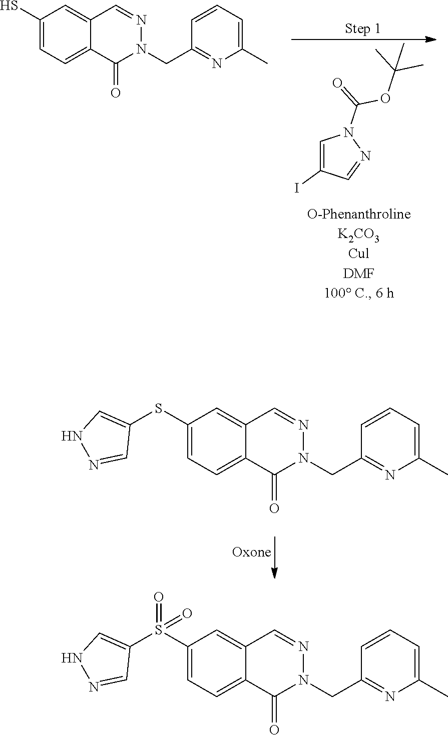 [SMILES: Cc1cccc(Cn2ncc3cc(S)ccc3c2=O)n1 ; CC(C)(C)OC(=O)n1cc(I)cn1 ; Cc1cccc(Cn2ncc3cc(Sc4cn[nH]c4)ccc3c2=O)n1 ; Cc1cccc(Cn2ncc3cc(S(=O)(=O)c4cn[nH]c4)ccc3c2=O)n1]

### Step 1

N₂ was bubbled through a mixture of 6-mercapto-2-((6-methylpyridin-2-yl)methyl)phthalazin-1(2H)-one (Intermediate 55, 100 mg, 1 Eq, 353 μmol), tert-butyl 4-iodo-1H-pyrazole-1-carboxylate (125 mg, 1.2 Eq, 424 μmol), 1,10-phenanthroline (25.4 mg, 0.4 Eq, 141 μmol) and potassium carbonate (73.2 mg, 1.5 Eq, 529 μmol) in DMF (1.5 mL) for 5 minutes. Copper(I) iodide (13.4 mg, 0.2 Eq, 70.6 μmol) was added and the mixture stirred under N₂ at 100° C. for 5.5 h. The mixture was allowed to cool to RT, then neutralized with 1 N HCl (0.5 mL). Water and DCM were added, and the layers separated. The organic layer was washed with brine, then absorbed on silica. The crude product was purified by chromatography to afford 6-((1H-pyrazol-4-yl)thio)-2-((6-methylpyridin-2-yl)methyl)phthalazin-1(2H)-one (57.0 mg) as a white solid.

### Step 2

A mixture of OXONE® (221 mg, 2.2 Eq, 359 μmol) and 6-((1H-pyrazol-4-yl)thio)-2-((6-methylpyridin-2-yl)methyl)phthalazin-1(2H)-one (57.0 mg, 1 Eq, 163 μmol) in DMF (1 mL) was stirred for 3 h. Sat. NaHCO₃, water and DCM were added, and the layers separated. The organic layer was washed with brine, then absorbed on silica. The crude product was purified by chromatography. The product was then dissolved in DCM and the resulting solution washed with water, brine. MeOH was added and the mixture dried (MgSO₄), then concentrated under vacuum to afford the title compound (36.4 mg as a white solid. ₁H NMR (DMSO-d6) δ: 13.89 (s, 1H), 8.64 (s, 1H), 8.63 (d, J=1.8 Hz, 1H), 8.44 (d, J=8.4 Hz, 1H), 8.32 (dd, J=8.4, 1.9 Hz, 2H), 7.59 (t, J=7.7, 7.7 Hz, 1H), 7.13 (d, J=7.7 Hz, 1H), 6.94 (d, J=7.8 Hz, 1H), 5.38 (s, 2H), 2.40 (s, 3H). MS (ES⁺): 382 (M+H)⁺

### Example 239—6-((1H-pyrazol-3-yl)sulfonyl)-2-((6-methylpyridin-2-yl)methyl)phthalazin-1(2H)-one

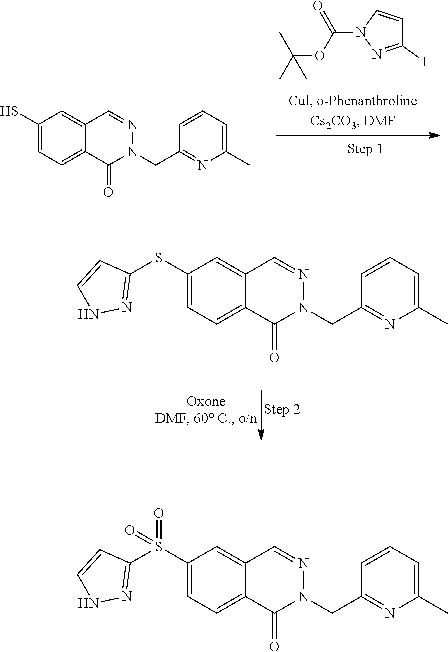 [SMILES: CC(C)(C)OC(=O)n1ccc(I)n1 ; Cc1cccc(Cn2ncc3cc(S)ccc3c2=O)n1 ; Cc1cccc(Cn2ncc3cc(Sc4cc[nH]n4)ccc3c2=O)n1 ; Cc1cccc(Cn2ncc3cc(S(=O)(=O)c4cc[nH]n4)ccc3c2=O)n1]

### Step 1

A mixture of 6-mercapto-2-((6-methylpyridin-2-yl)methyl)phthalazin-1(2H)-one (Intermediate 55, 250 mg), tert-butyl 3-iodo-1H-pyrazole-1-carboxylate (280 mg, 1.1 Eq, 951 μmol), o-phenanthroline (62.3 mg, 0.40 Eq, 346 μmol), and cesium carbonate (563 mg, 2.0 Eq, 1.73 mmol) in DMF (4.40 mL) was bubbled through with N₂ for 5 min before adding CuI (32.9 mg, 0.20 Eq, 173 μmol). The reaction mixture was further sparged for 5 minutes then heated at 100° C. for 7 h. The reaction mixture was cooled to RT and diluted with water (30 mL) before adding 1M HCl (3-4 mL). The aqueous was extracted with EtOAc (30 mL×4). The combined organics were washed with half saturated brine (50 mL×3), dried (MgSO₄) and concentrated under reduced pressure to give a yellow solid (262 mg). The crude product was purified by chromatography to afford 6-((1H-pyrazol-3-yl)thio)-2-((6-methylpyridin-2-yl)methyl)phthalazin-1(2H)-one (178.0 mg) as an off-white powder. ¹H NMR (400 MHz, DMSO-d6) δ 13.52 (s, 1H), 8.35 (s, 1H), 8.14 (d, J=8.5 Hz, 1H), 8.01 (s, 1H), 7.63-7.51 (m, 3H), 7.12 (d, J=7.7 Hz, 1H), 6.89 (d, J=7.8 Hz, 1H), 6.61 (s, 1H), 5.34 (s, 2H), 2.41 (s, 3H). MS (ES)⁺: 340 (M+H)⁺

### Step 2

A suspension of 6-((1H-pyrazol-3-yl)thio)-2-((6-methylpyridin-2-yl)methyl)phthalazin-1(2H)-one (100 mg, 80% Wt, 1 Eq, 229 μmol) and OXONE® (352 mg, 2.5 Eq, 572 μmol) in DMF (2.00 mL) was stirred at room temperature for 29 h. The reaction mixture was concentrated under reduced pressure (azeotroped with PhMe), diluted in DCM (10 mL) and filtered through celite washing with DCM (10 mL) followed by MeOH (10 mL). The filtrates were combined with the concentrated under reduced pressure to give a yellow oil. The oil was diluted in DCM (10 mL) with a few drops of MeOH, concentrated onto silica and purified by chromatography to afford the title compound (82.7 mg) as a white solid. ¹H NMR (400 MHz, DMSO-d6) δ 13.99 (s, 1H), 8.69 (s, 1H), 8.66 (d, J=1.8 Hz, 1H), 8.46 (d, J=8.4 Hz, 1H), 8.30 (dd, J=8.4, 1.9 Hz, 1H), 8.02 (dd, J=2.6, 1.1 Hz, 1H), 7.58 (t, J=7.7 Hz, 1H), 7.13 (d, J=7.7 Hz, 1H), 6.99-6.92 (m, 2H), 5.38 (s, 2H), 2.40 (s, 3H). MS (ES)⁺: 382 (M+H)⁺

### Example 240—2-((1H-pyrazol-3-yl)methyl)-6-((1-methyl-1H-pyrazol-4-yl)sulfonyl)phthalazin-1(2H)-one

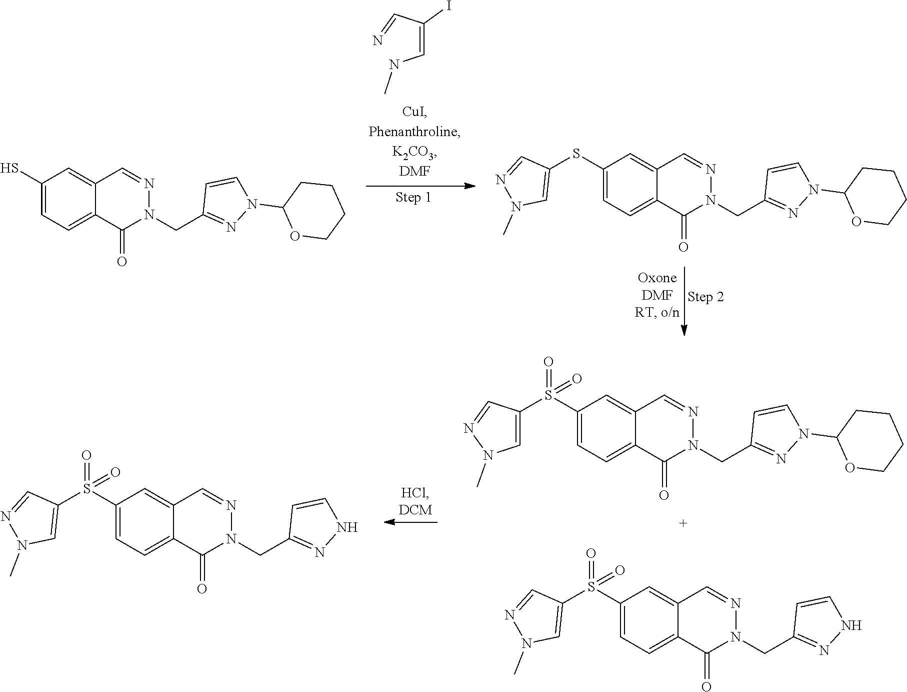

### Step 1

A solution of 6-mercapto-2-((1-(tetrahydro-2H-pyran-2-yl)-1H-pyrazol-3-yl)methyl)phthalazin-1(2H)-one (intermediate 49, 100 mg, 84% Wt, 1.0 Eq, 245 μmol), 1-methyl-4-iodo-1H-pyrazole (78.9 mg, 97% Wt, 1.5 Eq, 368 μmol), o-phenanthroline (17.7 mg, 15.4 μL, 0.40 Eq, 98.1 μmol), CuI (9.34 mg, 0.20 Eq, 49.1 μmol) and potassium carbonate (67.8 mg, 2.0 Eq, 491 μmol) in DMF (4.00 mL) was heated at 120° C. overnight. The reaction mixture was combined with another crude product obtained on 86 uM scale, diluted with EtOAc (5 mL) and filtered through celite. The celite pad was further washed with EtOAc (20 mL). The filtrate was washed with 1:1 v/v water/brine (15 mL×3), dried with MgSO₄, filtered and concentrated under reduced pressure to give an orange oil (209 mg). The crude was dissolved in DCM (5 mL), concentrated onto silica and purified by chromatography to afford 6-((1-methyl-1H-pyrazol-4-yl)thio)-2-((1-(tetrahydro-2H-pyran-2-yl)-1H-pyrazol-3-yl)methyl)phthalazin-1(2H)-one (143 mg) as a white solid. ¹H NMR (400 MHz, DMSO-d6) δ 8.32 (s, 1H), 8.17 (s, 1H), 8.13 (d, J=8.4 Hz, 1H), 7.77 (d, J=2.4 Hz, 1H), 7.70 (d, J=0.7 Hz, 1H), 7.56-7.48 (m, 2H), 6.14 (d, J=2.4 Hz, 1H), 5.30 (dd, J=10.2, 2.4 Hz, 1H), 5.23 (s, 2H), 3.93 (s, 3H), 3.90-3.85 (m, 1H), 3.61-3.53 (m, 1H), 2.09-1.96 (m, 1H), 1.93-1.78 (m, 2H), 1.70-1.54 (m, 1H), 1.53-1.45 (m, 2H). MS (ES)⁺: 423 (M+H)⁺

### Step 2

A suspension of 6-((1-methyl-1H-pyrazol-4-yl)thio)-2-((1-(tetrahydro-2H-pyran-2-yl)-1H-pyrazol-3-yl)methyl)phthalazin-1(2H)-one (70 mg, 96% Wt, 1 Eq, 0.16 mmol) and OXONE® (0.22 g, 2.2 Eq, 0.35 mmol) in DMF (2.00 mL) was stirred at RT overnight. The reaction mixture was partitioned with DCM (15 mL) and water (10 mL). The aqueous was extracted with DCM (20 ml×3) and the combined organics were washed with a 1:1 v/v mixture of brine and water (50 mL×3), filtered through a phase separator and concentrated under reduced pressure then azeotrope with PhMe to afford crude mixture of 6-((1-methyl-1H-pyrazol-4-yl)sulfonyl)-2-((1-(tetrahydro-2H-pyran-2-yl)-1H-pyrazol-3-yl)methyl)phthalazin-1(2H)-one (67.2 mg) and 2-((1H-pyrazol-3-yl)methyl)-6-((1-methyl-1H-pyrazol-4-yl)sulfonyl)phthalazin-1(2H)-one (67.2 mg), as off-white solids.

### Step 3

HCl (4M in 1,4-dioxane) (74 mg, 0.51 mL, 4 molar, 20 Eq, 2.0 mmol) was added to a solution of 6-((1-methyl-1H-pyrazol-4-yl)sulfonyl)-2-((1-(tetrahydro-2H-pyran-2-yl)-1H-pyrazol-3-yl)methyl)phthalazin-1(2H)-one (66 mg, 70% Wt, 1 Eq, 0.10 mmol) in DCM (1.50 mL). The reaction mixture was stirred at 40° C. for 5 h before adding HCl 4M in 1,4-dioxane (74 mg, 0.51 mL, 4 molar, 20 Eq, 2.0 mmol). The reaction was stirred at 40° C. for 1.5 h, then allowed to cool to RT. The reaction mixture was diluted in DCM (10 mL) and quenched with 2M aq. NaOH (5 mL) and water (5 mL). The aqueous was extracted with DCM (15 mL×3) and the combined organics were washed with brine (30 mL), filtered through a phase separator and concentrated under reduced pressure to give a white solid (47 mg). The crude was purified by chromatography to afford the title compound (36.2 mg) as a white solid. ¹H NMR (400 MHz, DMSO-d6) δ 12.66 (s, 1H), 8.62-8.57 (m, 2H), 8.55 (s, 1H), 8.44 (d, J=8.4 Hz, 1H), 8.29 (dd, J=8.5, 1.9 Hz, 1H), 8.01 (d, J=0.8 Hz, 1H), 7.62 (app s, 1H), 6.13 (d, J=2.2 Hz, 1H), 5.31 (s, 2H), 3.87 (s, 3H). MS (ES)⁺: 371 (M+H)⁺

### Example 241—2-((1H-pyrazol-3-yl)methyl)-6-((1-(2-hydroxyethyl)-1H-pyrazol-4-yl)sulfonyl)phthalazin-1(2H)-one

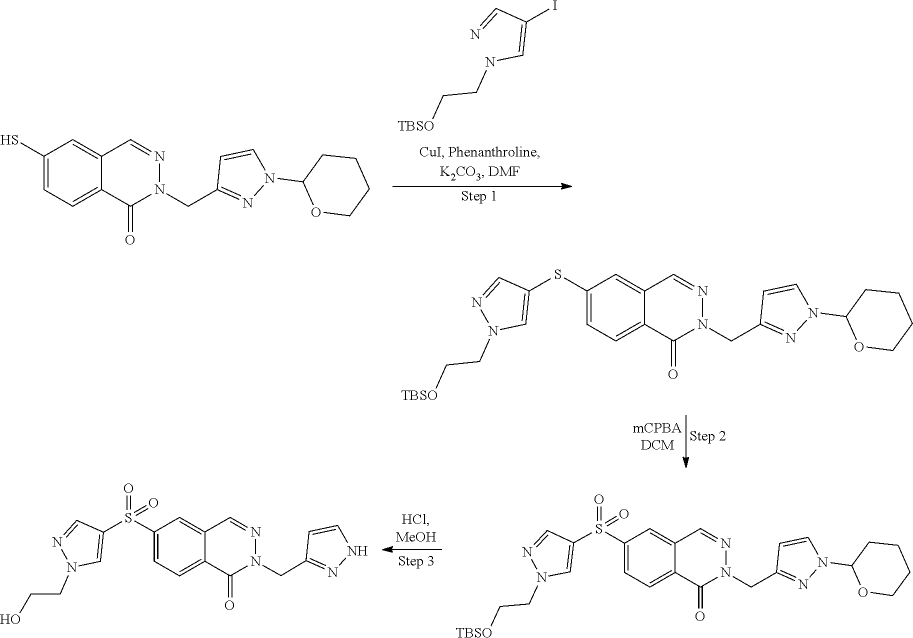 [SMILES: CC(C)(C)[Si](C)(C)OCCN/C=C(/I)C=N ; O=c1c2ccc(S)cc2cnn1Cc1ccn(C2CCCCO2)n1 ; CC(C)(C)[Si](C)(C)OCCn1cc(Sc2ccc3c(=O)n(Cc4ccn(C5CCCCO5)n4)ncc3c2)cn1 ; O=c1c2ccc(S(=O)(=O)c3cnn(CCO)c3)cc2cnn1Cc1cc[nH]n1 ; CC(C)(C)[Si](C)(C)OCCn1cc(S(=O)(=O)c2ccc3c(=O)n(Cc4ccn(C5CCCCO5)n4)ncc3c2)cn1]

### Step 1

N₂ was bubbled through a mixture of 6-mercapto-2-((1-(tetrahydro-2H-pyran-2-yl)-1H-pyrazol-3-yl)methyl)phthalazin-1(2H)-one (Intermediate 49, 75.0 mg, 1 Eq, 219 μmol), 1-(2-((tert-butyldimethylsilyl)oxy)ethyl)-4-iodo-1H-pyrazole (Intermediate 61, 92.6 mg, 1.2 Eq, 263 μmol), 1,10-phenanthroline (15.8 mg, 0.4 Eq, 87.6 μmol) and potassium carbonate (60.5 mg, 2 Eq, 438 μmol) in DMF (1.5 mL) for 5 min. Copper(I) iodide (8.34 mg, 0.2 Eq, 43.8 μmol) was added and the mixture was stirred at 100° C. under N₂ overnight, then allowed to cool to RT. Water and DCM were added and the layers separated. The organic layer was washed with brine, absorbed on silica and purified by chromatography to afford 6-((1-(2-((tert-butyldimethylsilyl)oxy)ethyl)-1H-pyrazol-4-yl)thio)-2-((1-(tetrahydro-2H-pyran-2-yl)-1H-pyrazol-3-yl)methyl)phthalazin-1(2H)-one (147 mg).

### Step 2

A mixture of mCPBA (141 mg, 70% Wt, 2.95 Eq, 574 μmol) and 6-((1-(2-((tert-butyldimethylsilyl)oxy)ethyl)-1H-pyrazol-4-yl)thio)-2-((1-(tetrahydro-2H-pyran-2-yl)-1H-pyrazol-3-yl)methyl)phthalazin-1(2H)-one (147 mg, 75% Wt, 1 Eq, 195 μmol) in DCM (1.50 mL) was stirred for 2 h. 2N NaOH and DCM were added and the layers separated. The organic layer was washed with brine, dried (MgSO₄) and concentrated under vacuum to afford 6-((1-(2-((tert-butyldimethylsilyl)oxy)ethyl)-1H-pyrazol-4-yl)sulfonyl)-2-((1-(tetrahydro-2H-pyran-2-yl)-1H-pyrazol-3-yl)methyl)phthalazin-1(2H)-one (102 mg).

### Step 3

Hydrogen chloride (4N in dioxane) (62.1 mg, 426 μL, 4.00 molar, 10 Eq, 1.70 mmol) was added to a mixture of 6-((1-(2-((tert-butyldimethylsilyl)oxy)ethyl)-1H-pyrazol-4-yl)sulfonyl)-2-((1-(tetrahydro-2H-pyran-2-yl)-1H-pyrazol-3-yl)methyl)phthalazin-1(2H)-one (102 mg, 1 Eq, 170 μmol) in MeOH (1.00 mL) and the mixture was stirred for 2 h, then concentrated under vacuum. Sat. NaHCO₃ and DCM were added and the layers separated. The precipitate that formed at the organic/aqueous phases interface was filtered, washed with water, MeCN then MTBE. The product was dried in a desiccator at 45° C. overnight to afford the title compound (20.7 mg) as a white solid. ¹H NMR (DMSO-d6) δ: 12.65 (s, 1H), 8.63-8.58 (m, 2H), 8.52 (s, 1H), 8.44 (d, J=8.5 Hz, 1H), 8.30 (dd, J=8.4, 1.9 Hz, 1H), 8.04 (s, 1H), 7.62 (s, 1H), 6.13 (d, J=2.1 Hz, 1H), 5.31 (s, 2H), 4.94 (t, J=5.3, 5.3 Hz, 1H), 4.18 (t, J=5.4, 5.4 Hz, 2H), 3.72 (td, J=5.3, 5.3, 5.2 Hz, 2H). MS (ES⁺): 401 (M+H)⁺

### Example 242—7-fluoro-2-((1-methyl-1H-pyrazol-3-yl)methyl)-6-(phenylsulfonyl)phthalazin-1(2H)-one

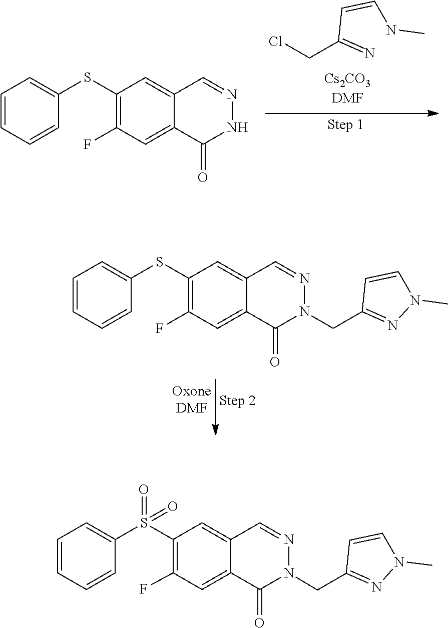

### Step 1

A suspension of 7-fluoro-6-(phenylthio)phthalazin-1(2H)-one (Intermediate 57, 17 mg, 95% Wt, 1 Eq, 59 μmol) and cesium carbonate (39 mg, 2 Eq, 0.12 mmol) in DMF (1 mL) was stirred at 80° C. for 30 min and then cooled to RT. A solution of 3-(chloromethyl)-1-methyl-1H-pyrazole (Intermediate 47, 8.0 mg, 1.0 Eq, 61 μmol) in DMF (1 mL) was added and the reaction mixture was stirred at RT for 18 h. The reaction mixture was concentrated in vacuo and the residue was azeotroped with toluene (2 times) to afford the crude product. The crude product was purified by chromatography to afford 7-fluoro-2-((1-methyl-1H-pyrazol-3-yl)methyl)-6-(phenylthio)phthalazin-1(2H)-one (9 mg) as a yellow solid. MS (ES⁺): 367 (M+H)⁺

### Step 2

A suspension of 7-fluoro-2-((1-methyl-1H-pyrazol-3-yl)methyl)-6-(phenylthio)phthalazin-1(2H)-one (9.0 mg, 93% Wt, 1 Eq, 23 μmol) and OXONE® (23 mg, 3.3 Eq, 75 μmol) in DMF (1 mL) was stirred at RT for 48 h and then heated to 60° C. for 24 h. The reaction mixture was diluted with DCM (5 mL) and washed with water (5 mL). The organic layer was collected and the aqueous was extracted with DCM (2×5 mL). The combined organic extracts were dried (phase separator) and concentrated in vacuo to afford the crude product. The crude product was purified by chromatography to afford the title compound (6 mg) as a yellow solid. ¹H NMR (400 MHz, DMSO-d6) δ 8.84 (d, J=6.7 Hz, 1H), 8.67 (d, J=0.7 Hz, 1H), 8.11 (d, J=10.0 Hz, 1H), 8.01 (dt, J=8.5, 1.2 Hz, 2H), 7.84-7.77 (m, 1H), 7.73-7.66 (m, 2H), 7.57 (d, J=2.2 Hz, 1H), 6.10 (d, J=2.2 Hz, 1H), 5.24 (s, 2H), 3.75 (s, 3H). MS (ES⁺): 399 (M+H)⁺

### Example 243—5-fluoro-2-((1-methyl-1H-pyrazol-3-yl)methyl)-6-(phenylsulfonyl)phthalazin-1(2H)-one

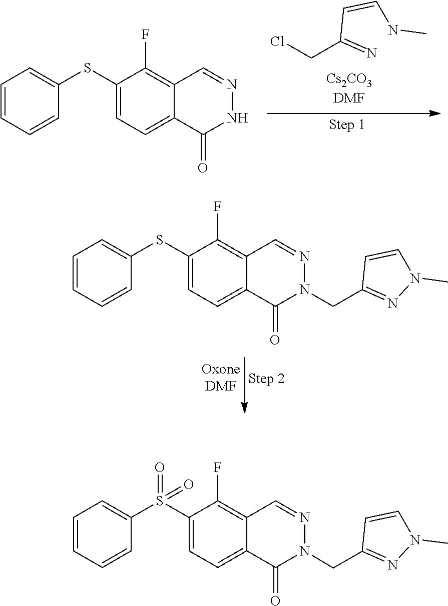

### Step 1

A suspension of 5-fluoro-6-(phenylthio)phthalazin-1(2H)-one (Intermediate 56, 12.0 mg, 80% Wt, 1 Eq, 35.3 μmol) and cesium carbonate (24.1 mg, 2.1 Eq, 74.0 μmol) in DMF (1 mL) was treated with 3-(chloromethyl)-1-methyl-1H-pyrazole (Intermediate 47, 12 mg, 10 μL, 2.6 Eq, 92 μmol) in one portion. The reaction mixture was stirred at RT for 18 h and then concentrated in vacuo to afford the crude product. The crude product was purified by chromatography on silica gel to afford 5-fluoro-2-((1-methyl-1H-pyrazol-3-yl)methyl)-6-(phenylthio)phthalazin-1(2H)-one (9.0 mg, 20 μmol) as a white solid. MS (ES⁺): 367 (M+H)⁺

### Step 2

A suspension of 5-fluoro-2-((1-methyl-1H-pyrazol-3-yl)methyl)-6-(phenylthio)phthalazin-1(2H)-one (9.0 mg, 80% Wt, 1 Eq, 20 μmol) and OXONE® (30 mg, 2.5 Eq, 49 μmol) in DMF (1 mL) was stirred at 50° C. for 20 h. Additional OXONE® (30 mg, 2.5 Eq, 49 μmol) was added and the mixture stirred at 50° C. for 3 h. Additional OXONE® (100 mg, 6.5 Eq, 163 μmol) was added and the mixture stirred at 50° C. for 2 h and then allowed to cool to RT. The reaction mixture was diluted with DCM (5 mL) and washed with sat. aq. NaHCO₃ (5 mL). The organic layer was collected and the aqueous was extracted with DCM (2×5 mL). The combined organic extracts were dried (phase separator) and concentrated in vacuo to afford the crude product. The crude product was purified by chromatography to afford the title compound (6.8 mg) as a white solid. ¹H NMR (400 MHz, DMSO-d6) δ 8.52 (d, J=0.8 Hz, 1H), 8.41 (dd, J=8.5, 6.8 Hz, 1H), 8.30 (d, J=8.5 Hz, 1H), 8.06-7.97 (m, 2H), 7.85-7.76 (m, 1H), 7.74-7.65 (m, 2H), 7.56 (d, J=2.2 Hz, 1H), 6.10 (dd, J=3.5, 2.2 Hz, 1H), 5.24 (s, 2H), 3.74 (s, 3H). MS (ES⁺): 399 (M+H)⁺

### Example 244—7-((2-hydroxyethyl)amino)-2-((1-methyl-1H-pyrazol-3-yl)methyl)-6-(phenylsulfonyl)phthalazin-1(2H)-one

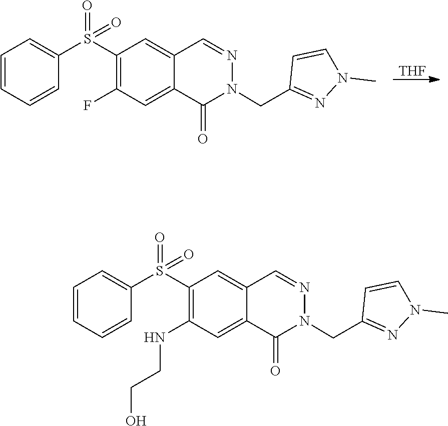 [SMILES: Cn1ccc(Cn2ncc3cc(S(=O)(=O)c4ccccc4)c(F)cc3c2=O)n1 ; Cn1ccc(Cn2ncc3cc(S(=O)(=O)c4ccccc4)c(NCCO)cc3c2=O)n1 ; "THF"]

A solution of 7-fluoro-2-((1-methyl-1H-pyrazol-3-yl)methyl)-6-(phenylsulfonyl)phthalazin-1(2H)-one (Example 242, 18 mg, 98% Wt, 1 Eq, 44 μmol) and ethanolamine (6.1 mg, 6.0 μL, 2.2 Eq, 99 μmol) in THF (2 mL) was heated in the microwave at 150° C. for 15 min. The reaction mixture was allowed to cool to RT and then concentrated in vacuo to afford the crude product. The crude product was purified by chromatography to afford the title compound (7.5 mg) as a pale yellow solid. ¹H NMR (400 MHz, DMSO-d6) δ 8.56 (s, 1H), 8.33 (s, 1H), 8.05-7.98 (m, 2H), 7.78-7.69 (m, 1H), 7.63 (t, J=7.8 Hz, 2H), 7.54 (d, J=2.2 Hz, 1H), 7.34 (s, 1H), 7.03 (t, J=5.1 Hz, 1H), 6.06 (d, J=2.2 Hz, 1H), 5.17 (s, 2H), 5.10 (t, J=4.9 Hz, 1H), 3.75 (s, 3H), 3.65-3.57 (m, 2H), 3.34-3.27 (m, 2H). MS (ES⁺): 440 (M+H)⁺

### Example 245—8-fluoro-2-((1-methyl-1H-pyrazol-3-yl)methyl)-6-(phenylsulfonyl)phthalazin-1(2H)-one

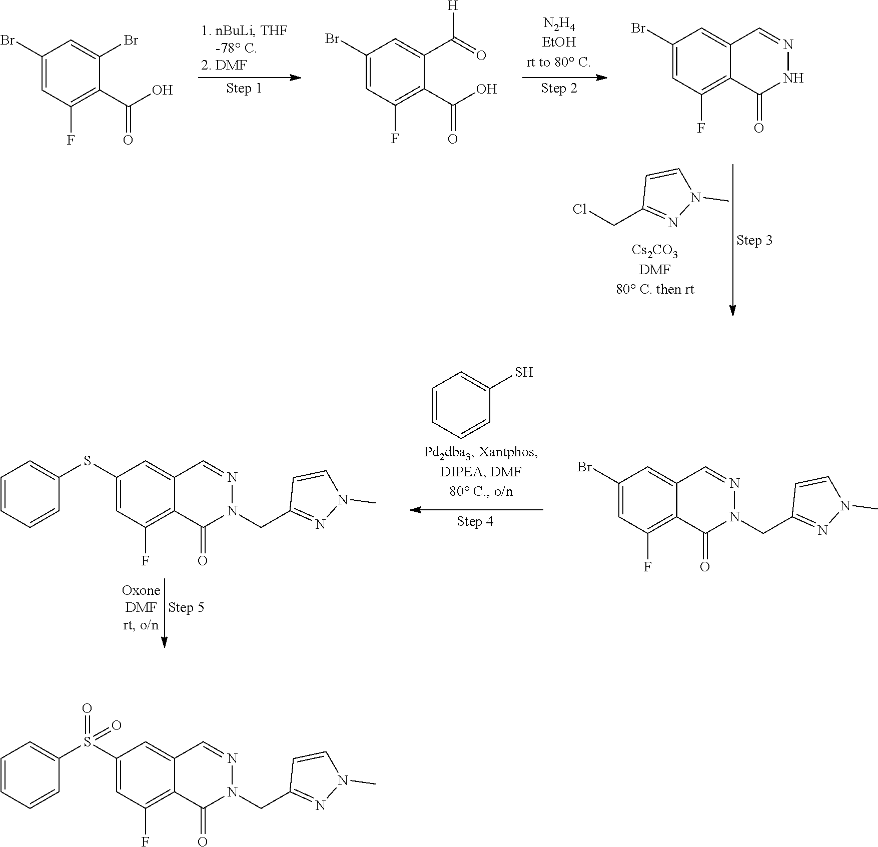

### Step 1

n-BuLi (1.9 M in hexanes) (243 mg, 2.00 mL, 1.90 molar, 2.26 Eq, 3.80 mmol) was added dropwise to THF (5 mL) under N₂ at −78° C. A solution of 2,4-dibromo-6-fluorobenzoic acid (500 mg, 1 Eq, 1.68 mmol) in THF (5 mL) was added dropwise at −78° C. and the reaction mixture was stirred at −78° C. for 15 minutes. DMF (0.65 mL) was added in one portion and the reaction mixture was allowed to warm to RT and stirred for 1 h. The reaction mixture was partitioned between EtOAc (20 mL) and 1 M HCl (aq.) (20 mL). The organic layer was collected and the aqueous was extracted with EtOAc (2×20 mL). The combined organic extracts were washed with brine (20 mL), dried (MgSO₄) and concentrated in vacuo to afford the crude product. The crude product was purified by chromatography to afford 4-bromo-2-fluoro-6-formylbenzoic acid (271 mg) as a pale yellow oil. MS (ES⁺): 247/249 (M+H)⁺

### Step 2

A solution of 4-bromo-2-fluoro-6-formylbenzoic acid (270 mg, 31% Wt, 1 Eq, 339 μmol) and hydrazine hydrate (35 wt % hydrazine) (102 mg, 100 μL, 35% Wt, 3.29 Eq, 1.11 mmol) in EtOH (5 mL) was stirred at RT for 30 min and then heated to 80° C. for 3 h. The reaction mixture was concentrated in vacuo and the residue was azeotroped with EtOH (2 times) to afford the crude product. The crude product was purified by chromatography to afford 6-bromo-8-fluorophthalazin-1(2H)-one (132 mg) as a pale yellow solid. MS (ES⁺): 243/245 (M+H)⁺

### Step 3

A stirred suspension of 6-bromo-8-fluorophthalazin-1(2H)-one (53 mg, 100% Wt, 1 Eq, 0.22 mmol) and cesium carbonate (0.14 g, 2.00 Eq, 0.44 mmol) in DMF (2 mL) was heated to 80° C. for 30 min and then allowed to cool to RT. 3-(chloromethyl)-1-methyl-1H-pyrazole (30 mg, 25 μL, 1.05 Eq, 0.23 mmol) was added and the reaction mixture was stirred at RT for 18 h. The reaction mixture was concentrated in vacuo and the residue was azeotroped with toluene (2 times) to afford the crude product. The crude product was purified by chromatography to afford 6-bromo-8-fluoro-2-((1-methyl-1H-pyrazol-3-yl)methyl)phthalazin-1(2H)-one (43 mg) as a pale yellow oil. MS (ES⁺): 337/339 (M+H)⁺

### Step 4

A solution of 6-bromo-8-fluoro-2-((1-methyl-1H-pyrazol-3-yl)methyl)phthalazin-1(2H)-one (43 mg, 63% Wt, 1 Eq, 80 μmol) and DIPEA (24 mg, 33 μL, 2.4 Eq, 0.19 mmol) in DMF (2 mL) was sparged with N₂ for 5 min and then Pd₂(dba)₃ (6 mg, 0.08 Eq, 7 μmol), xantphos (8 mg, 0.2 Eq, 0.01 mmol) and benzenethiol (17 mg, 16 μL, 2.0 Eq, 0.16 mmol) were added sequentially. The reaction mixture was stirred at 80° C. for 18 h. The reaction mixture was concentrated in vacuo and the residue was azeotroped with toluene (2 times) to afford the crude product. The crude product was purified by chromatography to afford 8-fluoro-2-((1-methyl-1H-pyrazol-3-yl)methyl)-6-(phenylthio)phthalazin-1(2H)-one (18 mg) as a pale yellow oil. MS (ES⁺): 367 (M+H)⁺

### Step 5

A suspension of 8-fluoro-2-((1-methyl-1H-pyrazol-3-yl)methyl)-6-(phenylthio)phthalazin-1(2H)-one (18 mg, 68% Wt, 1 Eq, 33 μmol) and OXONE® (75 mg, 3.7 Eq, 0.12 mmol) in DMF (1 mL) was stirred at RT for 20 h. The reaction mixture was diluted with DCM (5 mL) and washed with water (5 mL). The organic layer was collected and the aqueous was extracted with DCM (2×5 mL). The combined organic extracts were dried (phase separator) and concentrated in vacuo to afford the crude product. The crude product was purified by chromatography to afford the title compound (4.4 mg) as an off-white solid. ¹H NMR (400 MHz, DMSO-d6) δ 8.56 (d, J=2.4 Hz, 1H), 8.48-8.46 (m, 1H), 8.19-8.14 (m, 1H), 8.07 (dd, J=7.3, 1.7 Hz, 2H), 7.76 (t, J=7.5 Hz, 1H), 7.67 (dd, J=8.5, 6.9 Hz, 2H), 7.56 (d, J=2.2 Hz, 1H), 6.10 (d, J=2.2 Hz, 1H), 5.19 (s, 2H), 3.75 (s, 3H). MS (ES⁺): 399 (M+H)⁺

### Example 246—7-((2-hydroxyethyl)amino)-2-((2-hydroxypyridin-3-yl)methyl)-6-(phenylsulfonyl)phthalazin-1(2H)-one and Example 247—5-((2-hydroxyethyl)amino)-2-((2-hydroxypyridin-3-yl)methyl)-6-(phenylsulfonyl)phthalazin-1(2H)-one

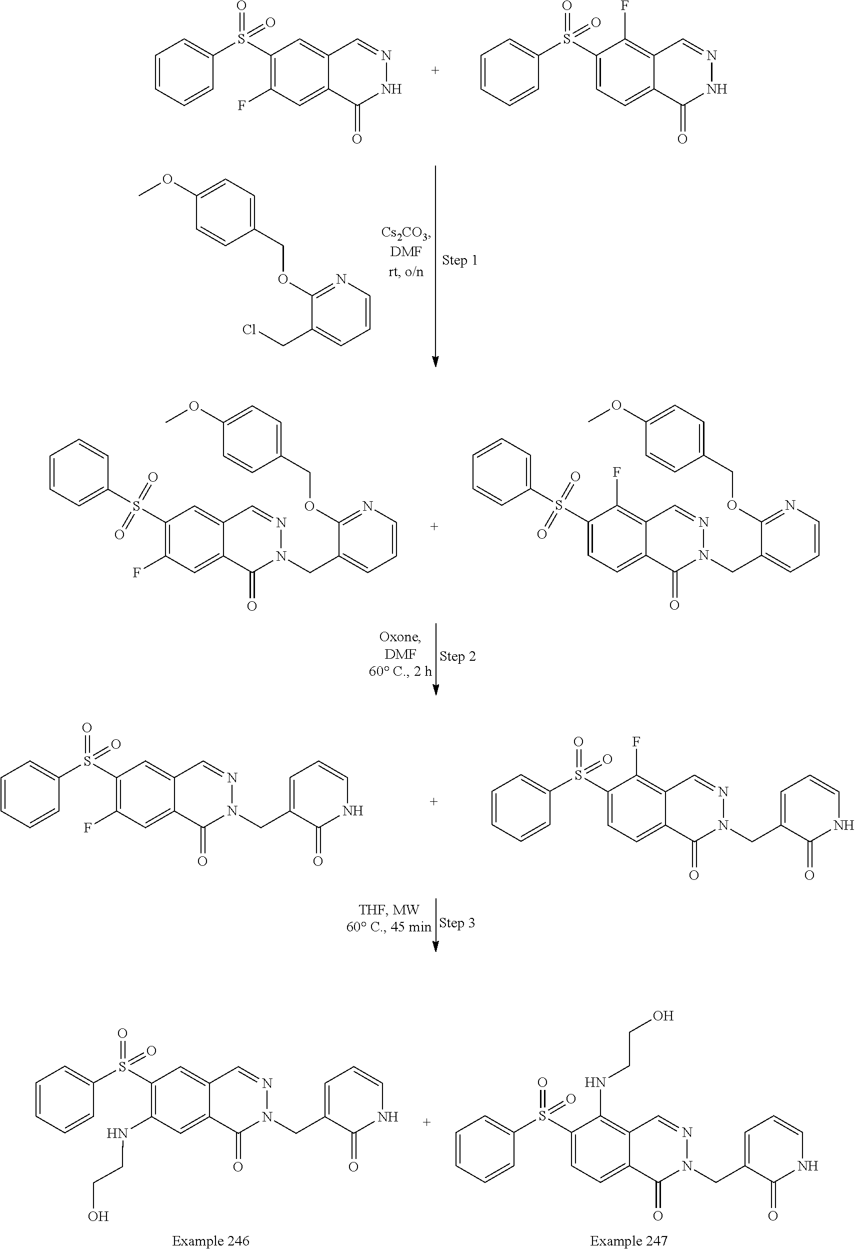

### Step 1

A suspension of 7-fluoro-6-(phenylthio)phthalazin-1(2H)-one (Intermediate 57, 100 mg, 67% Wt, 1 Eq, 246 μmol) and 5-fluoro-6-(phenylthio)phthalazin-1(2H)-one (Intermediate 56, 100 mg, 31% Wt, 0.463 Eq, 114 μmol) and cesium carbonate (241 mg, 3.0 Eq, 738 μmol) in DMF (1 mL) was stirred at RT for 5 min. A solution of 3-(chloromethyl)-2-((4-methoxybenzyl)oxy)pyridine (97.4 mg, 81.0 μL, 1.50 Eq, 369 μmol) in DMF (1 mL) was added and the reaction mixture was stirred at room temperature for 20 h, then concentrated in vacuo. The residue was azeotroped with toluene (3 times) to afford the crude product. The crude product was purified by chromatography to afford a mixture of 7-fluoro-2-((2-((4-methoxybenzyl)oxy)pyridin-3-yl)methyl)-6-(phenylthio)phthalazin-1(2H)-one (126 mg) and 5-fluoro-2-((2-((4-methoxybenzyl)oxy)pyridin-3-yl)methyl)-6-(phenylthio)phthalazin-1(2H)-one (126 mg, 63 μmo) as a yellow solid. MS (ES⁺): 500 (M+H)⁺

### Step 2

A suspension of 7-fluoro-2-((2-((4-methoxybenzyl)oxy)pyridin-3-yl)methyl)-6-(phenylthio)phthalazin-1(2H)-one (126 mg, 46% Wt, 1 Eq, 116 μmol) and 5-fluoro-2-((2-((4-methoxybenzyl)oxy)pyridin-3-yl)methyl)-6-(phenylthio)phthalazin-1(2H)-one (126 mg, 25% Wt, 0.543 Eq, 63.1 μmol) and OXONE® (461 mg, 6.46 Eq, 750 μmol) in DMF (2 mL) was stirred at 60° C. for 2 h. The reaction mixture was diluted with DCM (10 mL) and washed with sat. aq. NaHCO₃ (10 mL). The organic layer was collected and the aqueous was extracted with DCM (2×5 mL). The combined organic extracts were dried (phase separator) and concentrated in vacuo to afford the crude product. The crude product was purified by chromatography to afford a mixture of 7-fluoro-2-((2-hydroxypyridin-3-yl)methyl)-6-(phenylsulfonyl)phthalazin-1(2H)-one (47 mg) and 5-fluoro-2-((2-hydroxypyridin-3-yl)methyl)-6-(phenylsulfonyl)phthalazin-1(2H)-one (47 mg) as a yellow solid. MS (ES⁺): 412 (M+H)⁺

### Step 3

A solution of a 2:1 mixture of 7-fluoro-2-((2-hydroxypyridin-3-yl)methyl)-6-(phenylsulfonyl)phthalazin-1(2H)-one and 5-fluoro-2-((2-hydroxypyridin-3-yl)methyl)-6-(phenylsulfonyl)phthalazin-1(2H)-one (10 mg, 1 Eq, 24.3 μmol) and ethanolamine (4.0 mg, 4.0 μL, 4.5 Eq, 66 μmol) in THF (1 mL) was stirred in the microwave at 60° C. for 45 min. The reaction mixture was concentrated in vacuo to afford the crude product. The crude product was purified by preparative HPLC (chiral SFC on a Waters prep 100 with a PDA and a QDA detectors, 40° C., 120 bar. The column was a Chiralpak IH, 5 μM, 21 mm×250 mm; flow rate 65 mL/min of 40% MeOH (0.03% ammonia), 60% CO₂) to afford: 7-((2-hydroxyethyl)amino)-2-((2-hydroxypyridin-3-yl)methyl)-6-(phenylsulfonyl)phthalazin-1(2H)-one (Example 246, 2 mg) as a yellow solid. ¹H NMR (400 MHz, DMSO-d6) δ 11.68 (s, 1H), 8.59 (s, 1H), 8.38 (s, 1H), 8.07-8.00 (m, 2H), 7.79-7.70 (m, 1H), 7.64 (dd, J=8.4, 7.1 Hz, 2H), 7.35 (s, 1H), 7.29 (dd, J=6.5, 2.1 Hz, 1H), 7.06 (t, J=5.1 Hz, 1H), 6.94-6.87 (m, 1H), 6.06 (t, J=6.6 Hz, 1H), 5.11 (s, 1H), 5.01 (s, 2H), 3.64-3.60 (m, 2H), 3.35-3.27 (m, 2H). MS (ES⁺): 453 (M+H)⁺ and 5-((2-hydroxyethyl)amino)-2-((2-hydroxypyridin-3-yl)methyl)-6-(phenylsulfonyl)phthalazin-1(2H)-one (Example 247, 1.3 mg) was afforded as a yellow solid. ¹H NMR (400 MHz, DMSO-d6) δ 8.53 (s, 1H), 8.26 (d, J=8.5 Hz, 1H), 8.08-8.00 (m, 2H), 7.76-7.69 (m, 2H), 7.68-7.61 (m, 2H), 7.32 (d, J=6.3 Hz, 1H), 6.97 (d, J=21.1 Hz, 1H), 6.07-6.00 (m, 1H), 5.02 (s, 2H), 3.47 (s, 4H). 3 protons not observed in DMSO-d6. MS (ES⁺): 453 (M+H)⁺

### Example 248—3-((1-methyl-1H-pyrazol-3-yl)methyl)-7-(phenylsulfonyl)pyrido[3,4-d]pyridazin-4(3H)-one and Example 249—3-((1-methyl-1H-pyrazol-3-yl)methyl)-7-(phenylsulfinyl)pyrido[3,4-d]pyridazin-4(3H)-one

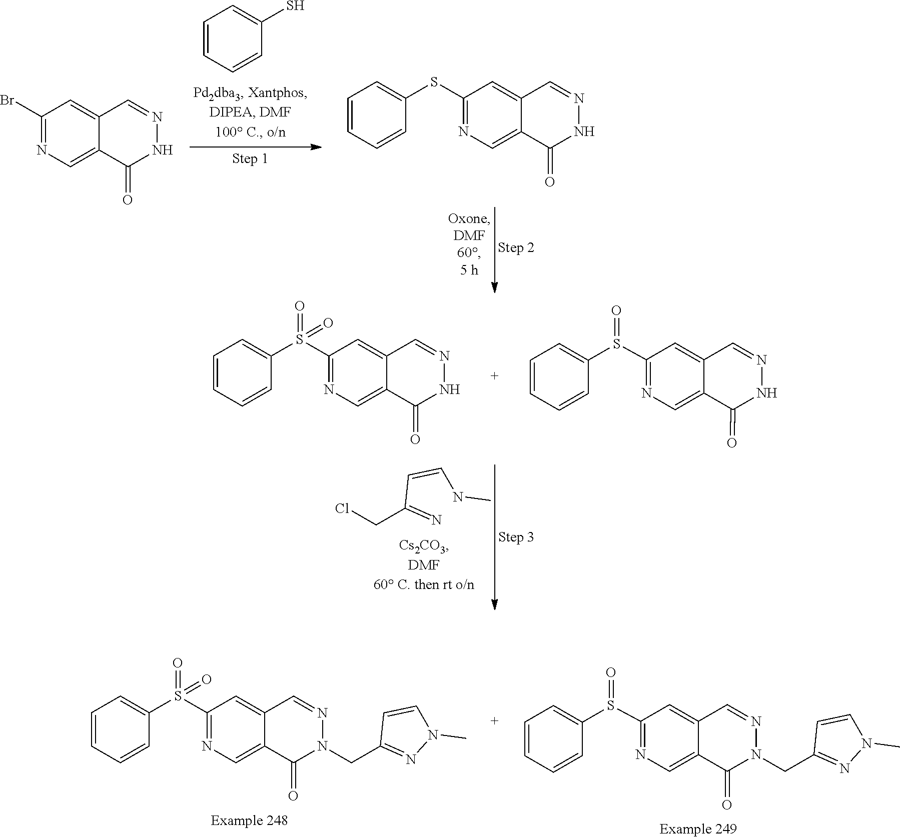

### Step 1

N₂ was bubbled through a mixture of 7-bromopyrido[3,4-d]pyridazin-4(3H)-one (500 mg, 1 Eq, 2.21 mmol), benzenethiol (244 mg, 227 μL, 1 Eq, 2.21 mmol) and N-ethyl-N-isopropylpropan-2-amine (343 mg, 462 μL, 1.2 Eq, 2.65 mmol) in DMF (10 mL) for 5 min. Pd₂dba₃ (101 mg, 0.05 Eq, 111 μmol) and Xantphos (128 mg, 0.1 Eq, 221 μmol) were added and the mixture was stirred at 100° C. under N₂ overnight, then allowed to cool to RT. Water and DCM were added and the layers separated. The organic layer was washed with brine, then absorbed on silica. The crude product was purified by chromatography to afford 7-(phenylthio)pyrido[3,4-d]pyridazin-4(3H)-one (519 mg). MS (ES⁺): 256 (M+H)⁺

### Step 2

A mixture of 7-(phenylthio)pyrido[3,4-d]pyridazin-4(3H)-one (100 mg, 1 Eq, 392 μmol) and OXONE® (482 mg, 2 Eq, 783 μmol) in DMF (2 mL) was stirred for 2.5 h, then stirred at 60° C. for 5 h. The mixture was allowed to cool to RT and stirred overnight. The mixture was poured into ice/water and the resulting precipitate was washed with water, then dried a vacuum oven at 40° C. overnight to afford 7-(phenylsulfinyl)pyrido[3,4-d]pyridazin-4(3H)-one-7-(phenylsulfonyl)pyrido[3,4-d]pyridazin-4(3H)-one (0.6/0.4) (84.0 mg) as a white solid. MS (ES⁺): 272; 288 (M+H)⁺

### Step 3

A stirred suspension of 7-(phenylsulfonyl)pyrido[3,4-d]pyridazin-4(3H)-one (84.0 mg, 53% Wt, 1 Eq, 155 μmol), 7-(phenylsulfinyl)pyrido[3,4-d]pyridazin-4(3H)-one (84.0 mg, 41% Wt, 0.819 Eq, 127 μmol) and cesium carbonate (151 mg, 3.0 Eq, 465 μmol) in DMF (2 mL) was heated to 80° C. for 1 h and then allowed to cool to RT. 3-(chloromethyl)-1-methyl-1H-pyrazole (50.6 mg, 42.2 μL, 2.5 Eq, 387 μmol) was added and the reaction mixture was stirred at RT for 20 h. The reaction mixture was diluted with DCM (10 mL) and washed with sat. aq. NaHCO₃ (10 mL). The organic layer was collected and the aqueous was extracted with DCM (2×5 mL). The combined organic extracts were dried (phase separator) and concentrated in vacuo to afford the crude product. The crude product was purified by chromatography to afford impure product. The crude product was purified by chromatography on RP Flash C18 (4 g cartridge, 1-45% (0.1% Formic acid in MeCN)/(0.1% Formic Acid in Water)) to afford: 3-((1-methyl-1H-pyrazol-3-yl)methyl)-7-(phenylsulfonyl)pyrido[3,4-d]pyridazin-4(3H)-one (Example 248, 23.8 mg) as a white solid, ¹H NMR (400 MHz, DMSO-d6) δ 9.49 (d, J=0.9 Hz, 1H), 8.79 (d, J=0.9 Hz, 1H), 8.70 (s, 1H), 8.05-7.98 (m, 2H), 7.81-7.74 (m, 1H), 7.68 (dd, J=8.5, 7.0 Hz, 2H), 7.58 (d, J=2.2 Hz, 1H), 6.12 (d, J=2.3 Hz, 1H), 5.27 (s, 2H), 3.75 (s, 3H); MS (ES⁺): 382 (M+H)⁺ and 3-((1-methyl-1H-pyrazol-3-yl)methyl)-7-(phenylsulfinyl)pyrido[3,4-d]pyridazin-4(3H)-one (Example 249, 24.0 mg) was isolated from the column as a white solid. ¹H NMR (400 MHz, DMSO-d6) δ 9.41 (s, 1H), 8.68 (s, 1H), 8.51 (d, J=0.9 Hz, 1H), 7.83-7.74 (m, 2H), 7.59-7.51 (m, 4H), 6.12 (d, J=2.2 Hz, 1H), 5.25 (d, J=1.7 Hz, 2H), 3.75 (s, 3H); MS (ES⁺): 366 (M+H)⁺.

### Example 250—7-((1-methyl-1H-pyrazol-3-yl)methyl)-3-(phenylsulfonyl)pyrido[2,3-d]pyridazin-8(7H)-one

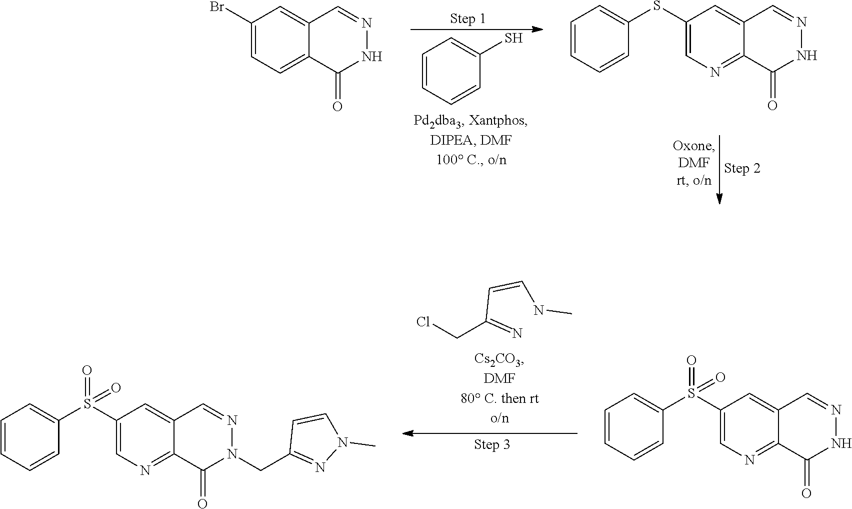

### Step 1

N₂ was bubbled through a mixture of 3-bromopyrido[2,3-d]pyridazin-8(7H)-one (500 mg, 1 Eq, 2.21 mmol), benzenethiol (244 mg, 227 μL, 1 Eq, 2.21 mmol) and N-ethyl-N-isopropylpropan-2-amine (343 mg, 462 μL, 1.2 Eq, 2.65 mmol) in DMF (4 mL) for 5 min. Pd₂dba₃ (101 mg, 0.05 Eq, 111 μmol) and Xantphos (128 mg, 0.1 Eq, 221 μmol) were added and the mixture was stirred at 100° C. under N₂ overnight, then allowed to cool to RT. Water and DCM were added and the layers separated through a phase separator. The organic layer was washed with brine, then absorbed on silica. The crude product was purified by chromatography to afford 3-(phenylthio)pyrido[2,3-d]pyridazin-8(7H)-one (316 mg). MS (ES⁺): 256 (M+H)⁺

### Step 2

A mixture of 3-(phenylthio)pyrido[2,3-d]pyridazin-8(7H)-one (100 mg, 1 Eq, 392 μmol) and OXONE® (482 mg, 2 Eq, 783 μmol) in DMF (2 mL) was stirred at RT overnight. The mixture was poured into ice/water and the resulting precipitate was washed with water, then dried a vacuum oven at 40° C. overnight to afford 3-(phenylsulfonyl)pyrido[2,3-d]pyridazin-8(7H)-one (71 mg). MS (ES⁺): 288 (M+H)⁺

### Step 3

A stirred suspension of 3-(phenylsulfonyl)pyrido[2,3-d]pyridazin-8(7H)-one (71 mg, 80% Wt, 1 Eq, 0.20 mmol) and cesium carbonate (0.19 g, 3.0 Eq, 0.59 mmol) in DMF (2 mL) was heated to 80° C. for 1 h and then allowed to cool to RT. 3-(chloromethyl)-1-methyl-1H-pyrazole (84 mg, 70 μL, 3.3 Eq, 0.64 mmol) was added and the reaction mixture was stirred at RT for 20 h. The reaction mixture was diluted with DCM (10 mL) and washed with sat. aq. NaHCO₃ (10 mL). The organic layer was collected and the aqueous was extracted with DCM (2×5 mL). The combined organic extracts were dried (phase separator) and concentrated in vacuo to afford the crude product. The crude product was purified by chromatography to afford the title compound (12.2 mg) as a white solid. ¹H NMR (400 MHz, CDCl₃) δ 9.45 (d, J=2.0 Hz, 1H), 8.61 (d, J=2.1 Hz, 1H), 8.27 (s, 1H), 8.05-7.97 (m, 2H), 7.67 (dd, J=8.5, 6.3 Hz, 1H), 7.58 (td, J=7.9, 2.0 Hz, 2H), 7.30 (d, J=2.2 Hz, 1H), 6.37 (d, J=2.2 Hz, 1H), 5.50 (s, 2H), 3.92 (s, 3H). MS (ES⁺): 382 (M+H)⁺

### Example 251—6-((1H-pyrazol-4-yl)sulfonyl)-2-((5-fluoro-6-methylpyridin-2-yl)methyl)phthalazin-1(2H)-one

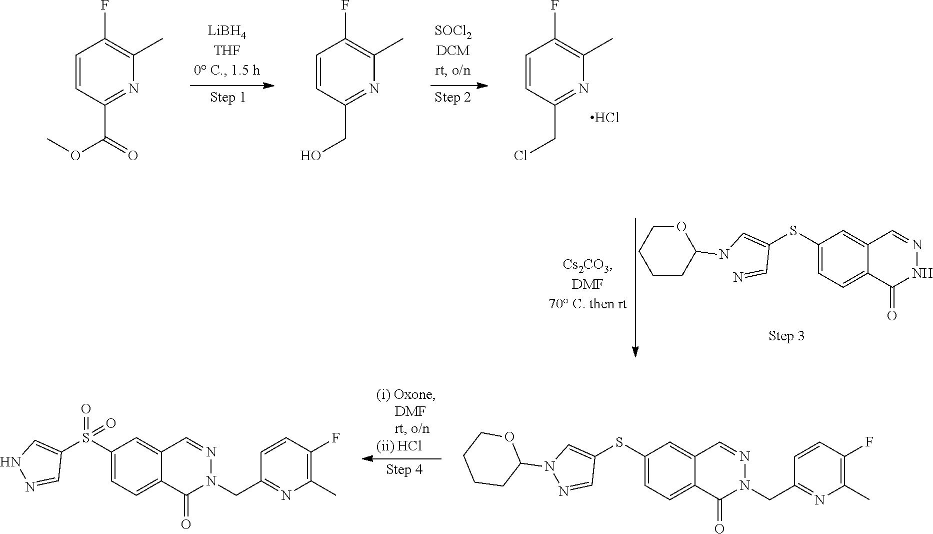

### Step 1

LiBH₄ (4M in THF) (38.3 mg, 440 μL, 4.00 molar, 1.2 Eq, 1.76 mmol) was added dropwise to a solution of methyl 5-fluoro-6-methylpicolinate (248 mg, 1 Eq, 1.47 mmol) in THF (5.00 mL) at 0° C. under N₂ and the mixture was allowed to warm to RT and stirred for 1.5 h. Sat. NH₄Cl, Rochelle's salt and EtOAc were added and the layers were separated. The aqueous layer was extracted with EtOAc and the combined organic extracts were washed with brine, dried (MgSO₄) and concentrated under vacuum to afford (5-fluoro-6-methylpyridin-2-yl)methanol (144 mg) as a brown oil that solidified on standing. MS (ES⁺): 142 (M+H)⁺

### Step 2

Thionyl chloride (39.9 mg, 24.3 μL, 1.25 Eq, 335 μmol) was added to a solution of (5-fluoro-6-methylpyridin-2-yl)methanol (39.7 mg, 1.05 Eq, 282 μmol) in DCM (1.50 mL) was stirred overnight. The mixture was concentrated under vacuum and the residue azeotroped with toluene, then MTBE to afford crude 6-(chloromethyl)-3-fluoro-2-methylpyridine hydrochloride (52.6 mg). The product was used without purification and characterization in the next step. Quantitative yield assumed.

### Step 3

A mixture of 6-((1-(tetrahydro-2H-pyran-2-yl)-1H-pyrazol-4-yl)thio)phthalazin-1(2H)-one (Intermediate 58, 80.0 mg, 1 Eq, 244 μmol) and cesium carbonate (238 mg, 3 Eq, 731 μmol) in DMF (1.25 mL) was stirred at 70° C. for 45 min. 2-(chloromethyl)-6-cyclopropylpyridine hydrochloride (59.7 mg, 1.2 Eq, 292 μmol) was added and the mixture stirred at RT for 3 days. Water and DCM were added and the layers separated. The organic layer was washed with brine, dried (MgSO₄) and concentrated under vacuum to afford crude 2-((6-cyclopropylpyridin-2-yl)methyl)-6-((1-(tetrahydro-2H-pyran-2-yl)-1H-pyrazol-4-yl)thio)phthalazin-1(2H)-one (112 mg). MS (ES⁺): 460 (M+H)⁺

### Step 4

A mixture of 2-((5-fluoro-6-methylpyridin-2-yl)methyl)-6-((1-(tetrahydro-2H-pyran-2-yl)-1H-pyrazol-4-yl)thio)phthalazin-1(2H)-one (108 mg, 70% Wt, 1 Eq, 168 μmol) and OXONE® (227 mg, 2.2 Eq, 369 μmol) in DMF (1.00 mL) was stirred at RT overnight. Sat. NaHCO₃ and DCM were added and the layers separated. The organic layer was washed with brine, dried and concentrated under vacuum to afford crude 2-((5-fluoro-6-methylpyridin-2-yl)methyl)-6-((1-(tetrahydro-2H-pyran-2-yl)-1H-pyrazol-4-yl)sulfonyl)phthalazin-1(2H)-one. The crude product was dissolved in MeOH (1.00 mL) and hydrogen chloride (4N in dioxane) (61.1 mg, 419 μL, 4.00 molar, 10 Eq, 1.68 mmol) was added. The mixture was stirred for 1.5 h, then concentrated under vacuum. Sat. NaHCO₃, 2N NaOH, 10% DCM:MeOH were added and the layers separated. The organic layer was washed with brine, then absorbed on silica. The crude product was purified by chromatography to afford the title compound (32.2 mg) as a white solid. ¹H NMR (DMSO-d6) δ: 13.91 (s, 1H), 8.63 (s, 1H), 8.62 (d, J=1.8 Hz, 1H), 8.43 (d, J=8.4 Hz, 1H), 8.38-8.28 (m, 2H), 8.31 (dd, J=8.5, 1.9 Hz, 1H), 7.58-7.49 (m, 1H), 7.12 (dd, J=8.5, 3.7 Hz, 1H), 5.38 (s, 2H), 2.37 (d, J=3.0 Hz, 3H). MS (ES⁺): 400 (M+H)⁺

### Example 252—6-((1H-pyrazol-4-yl)sulfonyl)-2-((3-fluoro-6-methylpyridin-2-yl)methyl)phthalazin-1(2H)-one

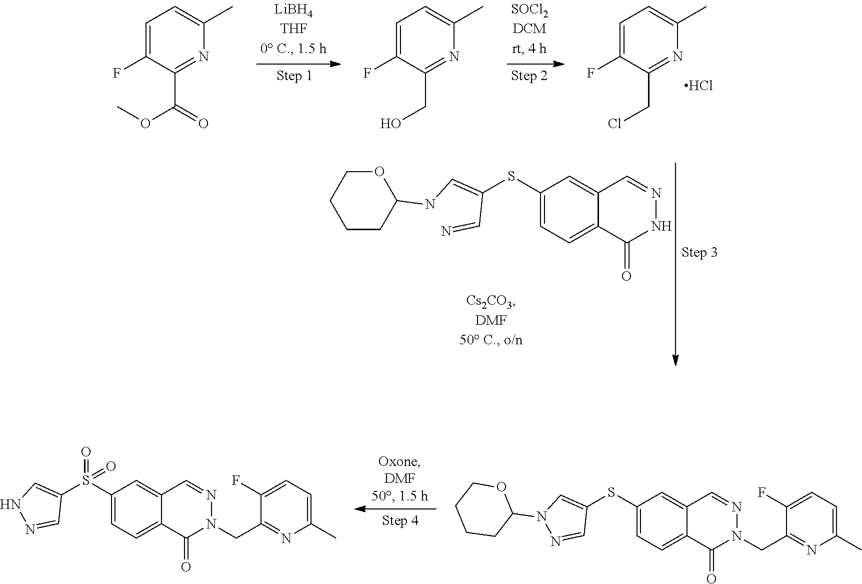

### Step 1

LiBH₄ (4M in THF) (23.2 mg, 266 μL, 4.00 molar, 1.2 Eq, 1.06 mmol) was added dropwise to a solution of methyl 3-fluoro-6-methylpicolinate (150 mg, 1 Eq, 887 μmol) in THF (5.00 mL) at 0° C. under N₂ and the mixture was allowed to warm to RT and stirred for 1.5 h. Sat. NH₄Cl, Rochelle's salt and EtOAc were added and the layers were separated. The aqueous layer was extracted with EtOAc and the combined organic extracts were washed with brine, dried (MgSO₄) and concentrated under vacuum to afford 3-fluoro-6-methylpyridin-2-yl)methanol (95.0 mg) as a white solid. MS (ES⁺): 142 (M+H)⁺

### Step 2

A solution of (3-fluoro-6-methylpyridin-2-yl)methanol (40.0 mg, 99% Wt, 1 Eq, 281 μmol) in DCM (1 mL) was treated with thionyl chloride (40.8 mg, 25.0 μL, 1.22 Eq, 343 μmol). The reaction mixture was stirred at RT for 4 h and then concentrated in vacuo. The residue was azeotroped with toluene to afford the crude product 2-(chloromethyl)-3-fluoro-6-methylpyridine, HCl (46.0 mg) pale yellow solid. The product was used without purification in the next step. MS (ES⁺): 195 (M+C4H8N+H)⁺

### Step 3

A suspension of 6-((1-(tetrahydro-2H-pyran-2-yl)-1H-pyrazol-4-yl)thio)phthalazin-1(2H)-one (Intermediate 58, 90.0 mg, 96% Wt, 1.12 Eq, 263 μmol) and cesium carbonate (229 mg, 3.0 Eq, 704 μmol) in DMF (1 mL) was stirred at 50° C. for 20 min. A solution of 2-(chloromethyl)-3-fluoro-6-methylpyridine, HCl (46.0 mg, 100% Wt, 1 Eq, 235 μmol) in DMF (1 mL) was added and the reaction mixture was stirred at 50° C. for 16 h. The reaction mixture was diluted with DCM (5 mL) and washed with sat. aq. NaHCO₃ (10 mL). The organic layer was collected and the aqueous was extracted with DCM (2×5 mL). The combined organic extracts were dried (phase separator) and concentrated in vacuo. The residue was azeotroped with toluene (3 times) to afford the crude product 2-((3-fluoro-6-methylpyridin-2-yl)methyl)-6-((1-(tetrahydro-2H-pyran-2-yl)-1H-pyrazol-4-yl)thio)phthalazin-1(2H)-one (130 mg, 0.23 mmol) as a yellow solid. The crude product was used without further purification. MS (ES⁺): 452 (M+H)⁺

### Step 4

A suspension of 2-((3-fluoro-6-methylpyridin-2-yl)methyl)-6-((1-(tetrahydro-2H-pyran-2-yl)-1H-pyrazol-4-yl)thio)phthalazin-1(2H)-one (130 mg, 81% Wt, 1 Eq, 233 μmol) and OXONE® (358 mg, 2.5 Eq, 583 μmol) in DMF (1 mL) and MeOH (0.5 mL) was stirred at 50° C. for 90 min. The reaction mixture was diluted with DCM (5 mL) and washed with sat. aq. NaHCO₃ (10 mL). The organic layer was collected and the aqueous was extracted with DCM (2×5 mL). The combined organic extracts were dried and concentrated in vacuo to afford the crude product. The crude product was purified by chromatography to afford the title compound (55.0 mg) as a white solid, after drying in vacuo (45° C., overnight). ¹H NMR (400 MHz, DMSO-d6) δ 13.91 (s, 1H), 8.61 (d, J=1.9 Hz, 1H), 8.59 (s, 1H), 8.42 (d, J=8.4 Hz, 1H), 8.36-8.29 (m, 2H), 8.31 (dd, J=8.5, 1.9 Hz, 1H), 7.59 (dd, J=10.0, 8.5 Hz, 1H), 7.23 (dd, J=8.5, 3.8 Hz, 1H), 5.47 (d, J=1.6 Hz, 2H), 2.30 (d, J=1.0 Hz, 3H). MS (ES⁺): 400 (M+H)⁺

### Example 253—6-(1H-pyrazol-4-ylsulfonyl)-2-((2,3-dihydrofuro[3,2-b]pyridin-5-yl)methyl)phthalazin-1(2H)-one

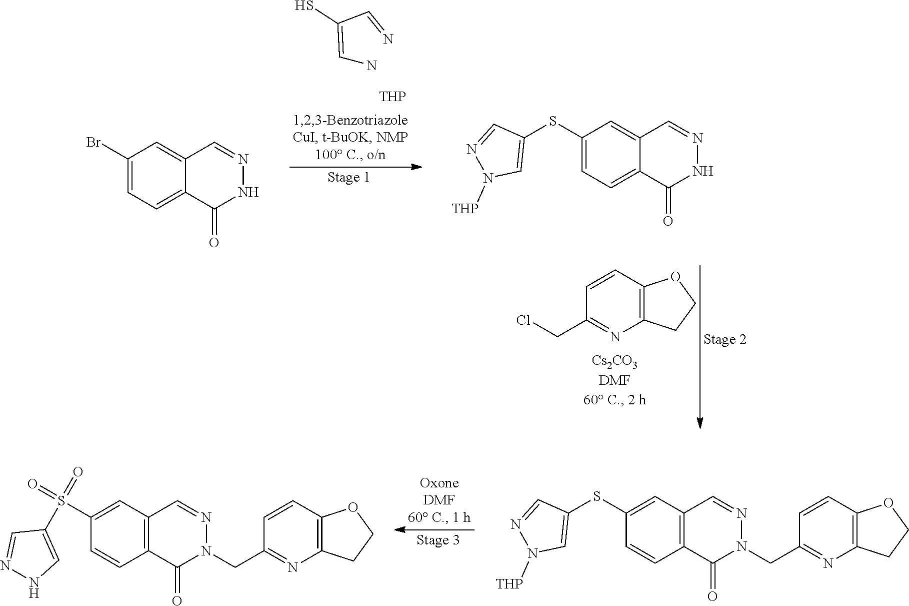

### Step 1

A mixture of 1-(tetrahydro-2H-pyran-2-yl)-1H-pyrazole-4-thiol (Intermediate 59, 2.4 g, 13.0 mmol), 6-bromophthalazin-1(2H)-one (2.24 g, 10.0 mmol), CuI (95 mg, 0.5 mmol) and 1,2,3-benztriazole (119 mg, 1.0 mmol) in NMP (20 mL) was added t-BuOK (2.24 g, 20.0 mmol) at RT. The reaction was stirred at 100° C. overnight under N₂. After LCMS indicated the reaction was complete, the reaction mixture was filtered through Celite and washed with EtOAc. The filtrate was diluted with water (50 mL) and extracted with EtOAc (20 mL×3). The combined organic layer was washed by brine, dried over Na₂SO₄, filtered and concentrated at 45° C. under reduced pressure. The residue was purified by flash column chromatography to give 6-(1-(tetrahydro-2H-pyran-2-yl)-1H-pyrazol-4-ylthio)phthalazin-1(2H)-one (2.6 g) as a yellow solid. MS (ES⁺): 329.2 (M+H)⁺

### Step 2

To the solution of 6-(1-(tetrahydro-2H-pyran-2-yl)-1H-pyrazol-4-ylthio)phthalazin-1(2H)-one (292 mg, 0.89 mmol), Cs₂CO₃ (868 mg, 2.67 mmol) in DMF (10 mL) was added 5-(chloromethyl)-2,3-dihydrofuro[3,2-b]pyridine (Intermediate 60, 150 mg, 0.89 mmol) stirred at 60° C. for 2 h. After LCMS indicated the reaction was complete, the mixture was extracted with DCM (5 mL×2). The organic layer was washed with brine, dried over Na₂SO₄, filtered and concentrated at 30° C. under reduced pressure. The residue was purified by flash column chromatography to give 2-((2,3-dihydrofuro[3,2-b]pyridin-5-yl)methyl)-6-(1-(tetrahydro-2H-pyran-2-yl)-1H-pyrazol-4-ylthio)phthalazin-1(2H)-one (160 mg) as a yellow solid. MS (ES⁺): 462.3 (M+H)⁺

### Step 3

A mixture of 2-((2,3-dihydrofuro[3,2-b]pyridin-5-yl)methyl)-6-(1-(tetrahydro-2H-pyran-2-yl)-1H-pyrazol-4-ylthio)phthalazin-1(2H)-one (160 mg, 0.34 mmol), OXONE® (528 mg, 0.86 mmol) in DMF (10 mL) stirred at 60° C. for 1 h. After LCMS indicated the reaction completed, the mixture was extracted with DCM (5 mL×2). The organic layer was washed with brine, dried over Na₂SO₄, filtered and concentrated at 30° C. under reduced pressure the residue was purified by prep-H PLC (Column: Waters X-SELECT C18 OBD 10 μm 19*250 mm; Flow Rate: 20 mL/min; solvent system: MeCN/(10 mmol/L NH4HCO3/water) gradient: MeCN: 40%-95%; collection wavelength: 214 nm). The fractions were concentrated at 42° C. under reduced pressure to remove MeCN, and the residue was lyophilized to give the title compound (81.96 mg) as white solid. ¹H NMR (400 MHz, DMSO-d6) δ: 13.91 (br, 1H), 8.61 (br, 3H), 8.45-8.42 (m, 1H), 8.33-8.29 (m, 2H), 8.06 (br, 1H), 7.07-7.04 (m, 1H), 6.99-6.96 (m, 1H), 5.34 (s, 2H), 4.59 (t, J=8.4 Hz, 2H), 3.18 (t, J=8.8 Hz, 2H). MS (ES⁺): 410.0 (M+H)⁺

The following compounds were made using similar procedures described above:

| Example | | |
|---|---|---|
| No. | Example Structure/Name | LCMS/¹H NMR data |
| 254 | 2-((1H-pyrazol-3-yl)methyl)-6-(pyridin-4-ylsulfonyl)phthalazin- 1(2H)-one 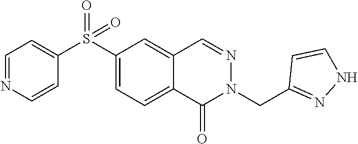 [SMILES: O=c1c2ccc(S(=O)(=O)c3ccncc3)cc2cnn1Cc1cc[nH]n1] | MS (ES⁺): 368 (M + H)⁺ ¹H NMR (400 MHz, DMSO-d6) δ 12.65 (s, 1H), 8.95-8.89 (m, 2H), 8.75 (d, J = 1.9 Hz, 1H), 8.61 (s, 1H), 8.47 (d, J = 8.5 Hz, 1H), 8.36 (dd, J = 8.4, 1.9 Hz, 1H), 8.00- 7.94 (m, 2H), 7.62 (s, 1H), 6.13 (d, J = 2.2 Hz, 1H), 5.31 (s, 2H). |
| 255 | 2-((2,4-dihydroxypyridin-3-yl)methyl)-6-(4- methoxyphenylsulfonyl)phthalazin-1(2H)-one 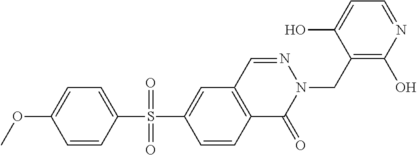 [SMILES: COc1ccc(S(=O)(=O)c2ccc3c(=O)n(Cc4c(O)ccnc4O)ncc3c2)cc1] | MS (ES⁺): 440.1 (M + H)⁺ ¹H NMR (400 MHz, CD₃OD) δ: 11.01 (br, 1H), 10.54 (br, 1H), 8.57 (d, J = 1.2 Hz, 1H), 8.48 (s, 1H), 8.42 (d, J = 8.8 Hz, 1H), 8.24 (dd, J = 8.4, 1.6 Hz, 1H), 7.96-7.94 (m, 2H), 7.18-7.15 (m, 3H), 5.87 (d, J = 7.2 Hz, 1H), 5.08 (s, 2H), 3.83 (s, 3H). |
| 256 | 2-((1-cyclopropyl-1H-pyrazol-3-yl)methyl)-6-(furo[3,2-b]pyridin- 5-ylsulfonyl)phthalazin-1(2H)-one 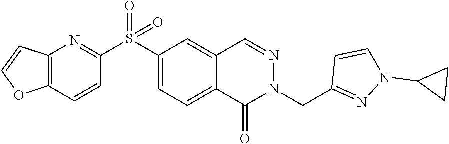 [SMILES: O=c1c2ccc(S(=O)(=O)c3ccc4occc4n3)cc2cnn1Cc1ccn(C2CC2)n1] | MS (ES⁺): 448 (M + H)⁺ ¹H NMR (400 MHz, DMSO-d6) δ 8.70 (d, J = 1.8 Hz, 1H), 8.64 (s, 1H), 8.57 (d, J = 2.4 Hz, 1H), 8.46 (d, J = 8.4 Hz, 1H), 8.42 (dd, J = 8.6, 1.0 Hz, 1H), 8.34 (dd, J = 8.4, 1.8 Hz, 1H), 8.29 (d, J = 8.6 Hz, 1H), 7.64 (d, J = 2.3 Hz, 1H), 7.27 (dd, J = 2.4, 1.0 Hz, 1H), 6.07 (d, J = 2.3 Hz, 1H), 5.24 (s, 2H), 3.61 (tt, J = 7.4, 3.7 Hz, 1H), 1.00-0.93 (m, 2H), 0.92-0.86 (m, 2H). |
| 257 | 2-((1H-pyrazol-3-yl)methyl)-6-((5-methoxypyridin-2- yl)sulfonyl)phthalazin-1(2H)-one 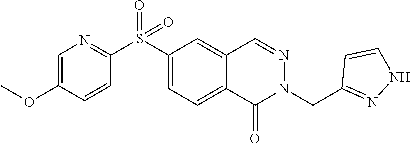 [SMILES: COc1ccc(S(=O)(=O)c2ccc3c(=O)n(Cc4cc[nH]n4)ncc3c2)nc1] | MS (ES⁺): 398 (M + H)⁺ ¹H NMR (400 MHz, DMSO-d6) δ 12.65 (s, 1H), 8.64 (d, J = 3.2 Hz, 2H), 8.45 (d, J = 8.4 Hz, 1H), 8.39 (d, J = 2.8 Hz, 1H), 8.27 (dd, J = 9.1, 7.2 Hz, 2H), 7.68 (dd, J = 8.8, 2.9 Hz, 1H), 7.62 (s, 1H), 6.13 (s, 1H), 5.30 (s, 2H), 3.91 (s, 3H). |
| 258 | 2-((1H-pyrazol-3-yl)methyl)-6-((2,3-dihydrofuro[2,3-c]pyridin-5- yl)sulfonyl)phthalazin-1(2H)-one 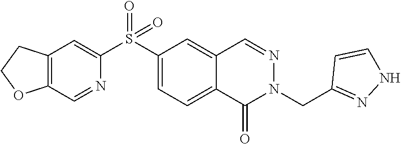 [SMILES: O=c1c2ccc(S(=O)(=O)c3cc4c(cn3)OCC4)cc2cnn1Cc1cc[nH]n1] | MS (ES⁺): 410 (M + H)⁺ ¹H NMR (400 MHz, DMSO-d6) δ 12.65 (s, 1H), 8.66-8.60 (m, 2H), 8.45 (d, J = 8.4 Hz, 1H), 8.26 (dd, J = 8.5, 1.8 Hz, 1H), 8.22 (s, 1H), 8.20 (s, 1H), 7.62 (s, 1H), 6.13 (s, 1H), 5.30 (s, 2H), 4.73 (t, J = 9.0 Hz, 2H), 3.36 (t, J = 9.0 Hz, 2H). |
| 259 | 6-((1-cyclopropyl-1H-pyrazol-4-yl)sulfonyl)-2-((6-methylpyridin- 2-yl)methyl)phthalazin-1(2H)-one 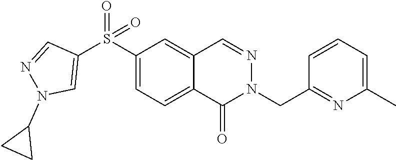 [SMILES: Cc1cccc(Cn2ncc3cc(S(=O)(=O)c4cnn(C5CC5)c4)ccc3c2=O)n1] | MS (ES⁺): 422 (M + H)⁺ ¹H NMR (400 MHz, DMSO-d6) δ 8.68 (s, 1H), 8.64 (s, 1H), 8.62 (d, J = 1.9 Hz, 1H), 8.44 (d, J = 8.4 Hz, 1H), 8.32 (dd, J = 8.4, 1.9 Hz, 1H), 8.02 (d, J = 0.8 Hz, 1H), 7.59 (t, J = 7.7 Hz, 1H), 7.13 (d, J = 7.6 Hz, 1H), 6.95 (d, J = 7.8 Hz, 1H), 5.39 (s, 2H), 3.83 (tt, J = 7.4, 3.8 Hz, 1H), 2.40 (s, 3H), 1.16-1.07 (m, 2H), 1.03-0.95 (m, 2H). |
| 260 | 2-((1H-pyrazol-3-yl)methyl)-6-(pyrazin-2-ylsulfonyl) phthalazin-1(2H)-one 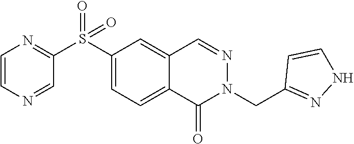 [SMILES: O=c1c2ccc(S(=O)(=O)c3cnccn3)cc2cnn1Cc1cc[nH]n1] | MS (ES⁺): 369 (M + H)⁺ ¹H NMR (400 MHz, DMSO-d6) δ 12.65 (s, 1H), 9.49 (d, J = 1.5 Hz, 1H), 9.01 (d, J = 2.4 Hz, 1H), 8.83 (dd, J = 2.4, 1.4 Hz, 1H), 8.74 (d, J = 1.8 Hz, 1H), 8.64 (s, 1H), 8.49 (dt, J = 8.5, 0.7 Hz, 1H), 8.38 (dd, J = 8.4, 1.9 Hz, 1H), 7.62 (s, 1H), 6.14 (s, 1H), 5.31 (s, 2H). |
| 261 | 2-(imidazo[1,2-a]pyridin-3-ylmethyl)-6- (phenylsulfonyl)phthalazin-1(2H)-one 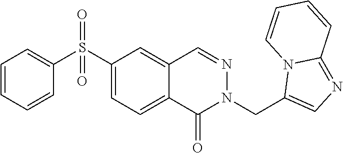 [SMILES: O=c1c2ccc(S(=O)(=O)c3ccccc3)cc2cnn1Cc1cnc2ccccn12] | MS (ES⁺): 417 (M + H)⁺ ¹H NMR (DMSO-d6) δ: 8.65 (d, J = 1.8 Hz, 1H), 8.63-8.60 (m, 1H), 8.59 (s, 1H), 8.45 (d, J = 8.5 Hz, 1H), 8.30 (dd, J = 8.5, 1.9 Hz, 1H), 8.04-7.97 (m, 2H), 7.77-7.68 (m, 1H), 7.68-7.60 (m, 3H), 7.60-7.53 (m, 1H), 7.25 (ddd, J = 9.1, 6.7, 1.3 Hz, 1H), 6.97 (ddd, J = 6.8, 6.8, 1.2 Hz, 1H), 5.69 (s, 2H). |
| 262 | 2-((1-methyl-1H-pyrazol-3-yl)methyl)-6- (phenylsulfonimidoyl)phthalazin-1(2H)-one 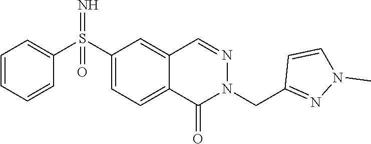 [SMILES: Cn1ccc(Cn2ncc3cc(S(=N)(=O)c4ccccc4)ccc3c2=O)n1] | MS (ES⁺): 380 (M + H)⁺ ¹H NMR (400 MHz, DMSO-d6) δ 8.62 (d, J = 1.8 Hz, 1H), 8.59 (s, 1H), 8.38 (d, J = 8.5 Hz, 1H), 8.30 (dd, J = 8.5, 1.8 Hz, 1H), 8.05- 7.97 (m, 2H), 7.67-7.56 (m, 3H), 7.55 (d, J = 2.2 Hz, 1H), 6.08 (d, J = 2.2 Hz, 1H), 5.39 (s, 1H), 5.24 (s, 2H), 3.74 (s, 3H). |
| 263 | 2-((1H-pyrazol-3-yl)methyl)-7-((2-hydroxyethyl)amino)-6- (phenylsulfonyl)phthalazin-1(2H)-one 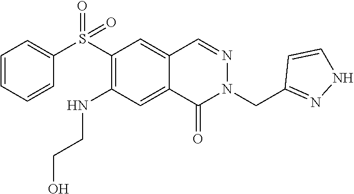 [SMILES: O=c1c2cc(NCCO)c(S(=O)(=O)c3ccccc3)cc2cnn1Cc1cc[nH]n1] | MS (ES⁺): 426 (M + H)⁺ ¹H NMR (400 MHz, DMSO-d6) δ 12.61 (br s, 1H), 8.56 (s, 1H), 8.35 (d, J = 6.8 Hz, 1H), 8.05-7.98 (m, 2H), 7.77-7.71 (m, 1H), 7.67-7.57 (m, 3H), 7.35 (s, 1H), 7.08- 6.98 (m, 1H), 6.09 (d, J = 2.3 Hz, 1H), 5.31- 5.16 (m, 2H), 5.10 (t, J = 4.9 Hz, 1H), 3.62 (q, J = 5.4 Hz, 2H), 3.33-3.28 (m, 2H). |
| 264 | 6-((1-(2-hydroxyethyl)-1H-pyrazol-4-yl)sulfonyl)-2-((1- methyl-1H-pyrazol-3-yl)methyl)phthalazin-1(2H)-one 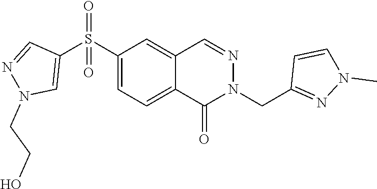 [SMILES: Cn1ccc(Cn2ncc3cc(S(=O)(=O)c4cnn(CCO)c4)ccc3c2=O)n1] | MS (ES⁺): 415 (M + H)⁺ ¹H NMR (DMSO-d6) δ: 8.60 (d, J = 1.8 Hz, 1H), 8.59 (s, 1H), 8.52 (s, 1H), 8.44 (d, J = 8.4 Hz, 1H), 8.30 (dd, J = 8.5, 1.8 Hz, 1H), 8.04 (s, 1H), 7.57 (d, J = 2.2 Hz, 1H), 6.10 (d, J = 2.2 Hz, 1H), 5.25 (s, 2H), 4.94 (t, J = 5.3, 5.3 Hz, 1H), 4.18 (t, J = 5.4, 5.4 Hz, 2H), 3.75 (s, 3H), 3.72 (td, J = 5.4, 5.4, 5.4 Hz, 2H). |
| 265 | 2-((1-cyclopropyl-1H-pyrazol-3-yl)methyl)-7-((2- hydroxyethyl)amino)-6-(phenylsulfonyl)phthalazin- 1(2H)-one 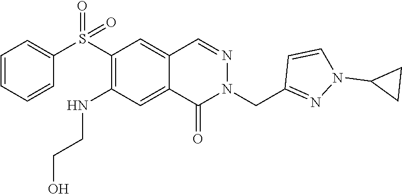 [SMILES: O=c1c2cc(NCCO)c(S(=O)(=O)c3ccccc3)cc2cnn1Cc1ccn(C2CC2)n1] | MS (ES⁺): 426 (M + H)⁺ ¹H NMR (400 MHz, DMSO-d6) δ 12.61 (br s, 1H), 8.56 (s, 1H), 8.35 (d, J = 6.8 Hz, 1H), 8.05-7.98 (m, 2H), 7.77-7.71 (m, 1H), 7.67-7.57 (m, 3H), 7.35 (s, 1H), 7.08-6.98 (m, 1H), 6.09 (d, J = 2.3 Hz, 1H), 5.31-5.16 (m, 2H), 5.10 (t, J = 4.9 Hz, 1H), 3.62 (q, J = 5.4 Hz, 2H), 3.33- 3.28 (m, 2H). |
| 266 | 3-((1H-pyrazol-3-yl)methyl)-7-((1-methyl-1H-pyrazol-4- yl)sulfonyl)pyrido[3,4-d]pyridazin-4(3H)-one 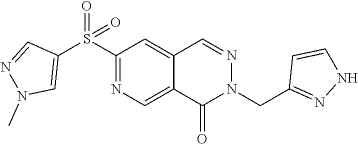 [SMILES: Cn1cc(S(=O)(=O)c2cc3cnn(Cc4cc[nH]n4)c(=O)c3cn2)cn1] | MS (ES⁺): 372 (M + H)⁺ ¹H NMR (DMSO-d6) δ: 12.68 (s, 1H), 9.52 (d, J = 1.0 Hz, 1H), 8.68 (d, J = 15.0 Hz, 2H), 8.57 (s, 1H), 7.96 (s, 1H), 7.64 (s, 1H), 6.16 (s, 1H), 5.33 (s, 2H), 3.90 (s, 3H). |
| 267 | 7-((1H-pyrazol-3-yl)methyl)-3-((1-methyl-1H-pyrazol-4- yl)sulfonyl)pyrido[2,3-d]pyridazin-8(7H)-one 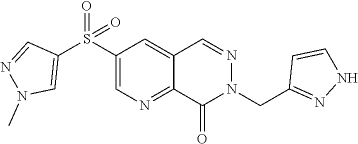 [SMILES: Cn1cc(S(=O)(=O)c2cnc3c(=O)n(Cc4cc[nH]n4)ncc3c2)cn1] | MS (ES⁺): 372 (M + H)⁺ ¹H NMR (400 MHz, DMSO-d6) δ 12.66 (s, 1H), 9.49 (d, J = 2.3 Hz, 1H), 9.01 (d, J = 2.3 Hz, 1H), 8.61 (s, 1H), 8.58 (s, 1H), 8.08 (d, J = 0.8 Hz, 1H), 7.63 (s, 1H), 6.15 (s, 1H), 5.33 (s, 2H), 3.88 (s, 3H). |
| 268 | 2-((1H-pyrazol-3-yl)methyl)-7-((2-hydroxyethyl)amino)-6-((1- methyl-1H-pyrazol-4-yl)sulfonyl)phthalazin-1(2H)-one 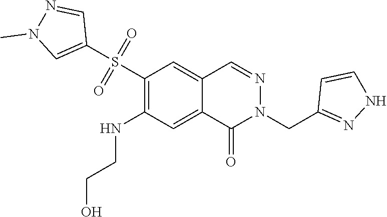 [SMILES: Cn1cc(S(=O)(=O)c2cc3cnn(Cc4cc[nH]n4)c(=O)c3cc2NCCO)cn1] | MS (ES⁺): 430 (M + H)⁺ ¹H NMR (400 MHz, DMSO-d6) δ 12.62 (s, 1H), 8.51 (s, 1H), 8.43 (s, 1H), 8.33 (s, 1H), 8.03 (s, 1H), 7.60 (s, 1H), 7.39 (s, 1H), 6.93- 6.87 (m, 1H), 6.08 (s, 1H), 5.23 (s, 2H), 5.12 (t, J = 4.9 Hz, 1H), 3.86 (s, 3H), 3.69 (q, J = 5.3 Hz, 2H), 3.38 (q, J = 5.3 Hz, 2H). |
| 269 | 2-((1H-pyrazol-3-yl)methyl)-6-((1-(2-hydroxyethyl)-1H-pyrazol-4- yl)sulfonyl)phthalazin-1(2H)-one 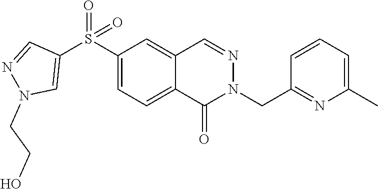 [SMILES: Cc1cccc(Cn2ncc3cc(S(=O)(=O)c4cnn(CCO)c4)ccc3c2=O)n1] | MS (ES⁺): 426 (M + H)⁺ ¹H NMR (DMSO-d6) δ: 8.67-8.62 (m, 2H), 8.54 (s, 1H), 8.44 (d, J = 8.4 Hz, 1H), 8.32 (dd, J = 8.4, 1.9 Hz, 1H), 8.06 (s, 1H), 7.59 (dd, J = 7.7, 7.7 Hz, 1H), 7.13 (d, J = 7.7 Hz, 1H), 6.95 (d, J = 7.8 Hz, 1H), 5.39 (s, 2H), 4.95 (t, J = 5.3, 5.3 Hz, 1H), 4.19 (t, J = 5.3, 5.3 Hz, 2H), 3.73 (td, J = 5.3, 5.3, 5.3 Hz, 2H), 2.40 (s, 3H). |
| 270 | 2-((6-methylpyridin-2-yl)methyl)-6-((1-(oxetan-3-yl)-1H-pyrazol-4- yl)sulfonyl)phthalazin-1(2H)-one 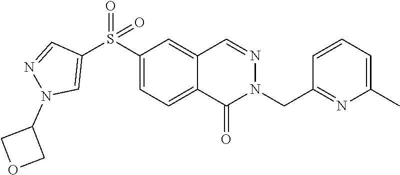 [SMILES: Cc1cccc(Cn2ncc3cc(S(=O)(=O)c4cnn(C5COC5)c4)ccc3c2=O)n1] | MS (ES⁺): 438 (M + H)⁺ ¹H NMR (DMSO-d6) δ: 8.73 (s, 1H), 8.67-8.62 (m, 2H), 8.45 (d, J = 8.4 Hz, 1H), 8.33 (dd, J = 8.4, 1.9 Hz, 1H), 8.20 (s, 1H), 7.59 (dd, J = 7.7, 7.7 Hz, 1H), 7.13 (d, J = 7.6 Hz, 1H), 6.95 (d, J = 7.7 Hz, 1H), 5.62 (tt, J = 13.7, 6.1 Hz, 1H), 5.39 (s, 2H), 4.88 (dd, J = 7.2, 7.2 Hz, 2H), 4.84 (dd, J = 6.6, 6.6 Hz, 2H), 2.40 (s, 3H). |
| 271 | 6-((1-(2-hydroxyethyl)-1H-pyrazol-4-yl)sulfonyl)-2-((6- methoxypyridin-3-yl)methyl)phthalazin-1(2H)-one 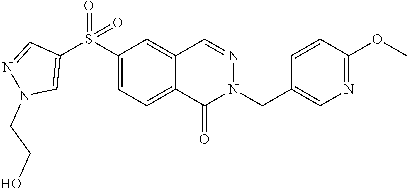 [SMILES: COc1ccc(Cn2ncc3cc(S(=O)(=O)c4cnn(CCO)c4)ccc3c2=O)cn1] | MS (ES⁺): 442 (M + H)⁺ ¹H NMR (400 MHz, DMSO-d6) δ: 8.62 (s, 1H), 8.60 (d, J = 1.8 Hz, 1H), 8.51 (s, 1H), 8.43 (d, J = 8.4 Hz, 1H), 8.30 (dd, J = 8.4, 1.9 Hz, 1H), 8.19 (d, J = 2.4 Hz, 1H), 8.03 (d, J = 0.7 Hz, 1H), 7.68 (dd, J = 8.6, 2.5 Hz, 1H), 6.76 (d, J = 8.5 Hz, 1H), 5.28 (s, 2H), 4.93 (t, J = 5.3, 5.3 Hz, 1H), 4.17 (t, J = 5.3, 5.3 Hz, 2H), 3.81 (s, 3H), 3.71 (td, J = 5.3, 5.3, 5.3 Hz, 2H). |
| 272 | 2-((1-cyclopropyl-1H-pyrazol-3-yl)methyl)-6-((1-(2-hydroxyethyl)- 1H-pyrazol-4-yl)sulfonyl)phthalazin-1(2H)-one 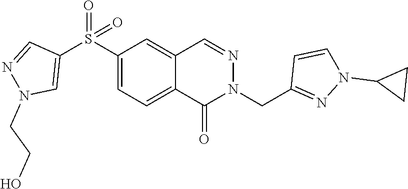 [SMILES: O=c1c2ccc(S(=O)(=O)c3cnn(CCO)c3)cc2cnn1Cc1ccn(C2CC2)n1] | MS (ES⁺): 441 (M + H)⁺ ¹H NMR (400 MHz, DMSO-d6) δ 8.61 (d, J = 1.8 Hz, 1H), 8.60 (s, 1H), 8.52 (s, 1H), 8.44 (d, J = 8.4 Hz, 1H), 8.30 (dd, J = 8.4, 1.9 Hz, 1H), 8.04 (s, 1H), 7.64 (d, J = 2.3 Hz, 1H), 6.07 (d, J = 2.3 Hz, 1H), 5.24 (s, 2H), 4.94 (t, J = 5.3 Hz, 1H), 4.18 (t, J = 5.4 Hz, 2H), 3.72 (td, J = 5.4, 5.4 Hz, 2H), 3.67-3.57 (m, 1H), 1.01-0.85 (m, 4H). |
| 273 | 2-((1-(oxetan-3-yl)-1H-pyrazol-3-yl)methyl)-6- (phenylsulfonyl)phthalazin-1(2H)-one 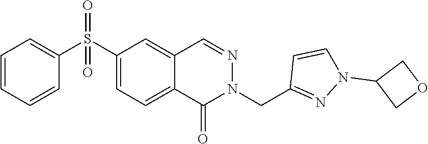 [SMILES: O=c1c2ccc(S(=O)(=O)c3ccccc3)cc2cnn1Cc1ccn(C2COC2)n1] | MS (ES⁺): 423 (M + H)⁺ ¹H NMR (400 MHz, DMSO-d6) δ 8.67 (d, J = 1.8 Hz, 1H), 8.60 (s, 1H), 8.43 (d, J = 8.5 Hz, 1H), 8.31 (dd, J = 8.5, 1.9 Hz, 1H), 8.06-7.99 (m, 2H), 7.78-7.70 (m, 1H), 7.70-7.62 (m, 2H), 7.52 (d, J = 1.8 Hz, 1H), 6.24 (d, J = 1.8 Hz, 1H), 5.91 (p, J = 7.1 Hz, 1H), 5.38 (s, 2H), 4.91 (dd, J = 6.3, 6.3 Hz, 2H), 4.84 (dd, J = 7.7, 6.2 Hz, 2H). |
| 274 | 2-((6-(1-hydroxyethyl)pyridin-2-yl)methyl)-6-((1-methyl-1H- pyrazol-4-yl)sulfonyl)phthalazin-1(2H)-one 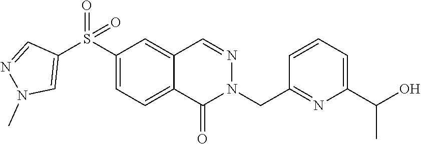 [SMILES: CC(O)c1cccc(Cn2ncc3cc(S(=O)(=O)c4cnn(C)c4)ccc3c2=O)n1] | MS (ES⁺): 426 (M + H)⁺ ¹H NMR (400 MHz, DMSO-d6) δ 8.65 (s, 1H), 8.63 (d, J = 1.9 Hz, 1H), 8.57 (s, 1H), 8.44 (d, J = 8.4 Hz, 1H), 8.31 (dd, J = 8.5, 1.9 Hz, 1H), 8.03 (d, J = 0.8 Hz, 1H), 7.69 (t, J = 7.8 Hz, 1H), 7.38 (d, J = 7.8 Hz, 1H), 6.99 (d, J = 7.7 Hz, 1H), 5.41 (s, 2H), 5.30 (d, J = 4.6 Hz, 1H), 4.66-4.59 (m, 1H), 3.88 (s, 3H), 1.28 (d, J = 6.6 Hz, 3H). |
| 275 | 2-((6-cyclopropylpyridin-2-yl)methyl)-6-((1-methyl-1H- pyrazol-4-yl)sulfonyl)phthalazin-1(2H)-one 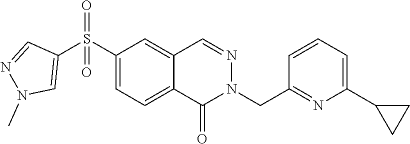 [SMILES: Cn1cc(S(=O)(=O)c2ccc3c(=O)n(Cc4cccc(C5CC5)n4)ncc3c2)cn1] | MS (ES⁺): 422 (M + H)⁺ ¹H NMR (400 MHz, DMSO-d6) δ 8.66- 8.60 (m, 2H), 8.57 (s, 1H), 8.44 (d, J = 8.4 Hz, 1H), 8.31 (dd, J = 8.4, 1.9 Hz, 1H), 8.03 (d, J = 0.8 Hz, 1H), 7.55 (t, J = 7.7 Hz, 1H), 7.13 (dd, J = 7.8, 1.0 Hz, 1H), 6.90 (dd, J = 7.7, 0.9 Hz, 1H), 5.35 (s, 2H), 3.88 (s, 3H), 2.00 (tt, J = 8.2, 4.8 Hz, 1H), 0.81 (dt, J = 8.1, 3.1 Hz, 2H), 0.69 (dt, J = 4.8, 3.1 Hz, 2H). |
| 276 | 6-((1-(azetidin-3-yl)-1H-pyrazol-4-yl)sulfonyl)-2-((6-methylpyridin- 2-yl)methyl)phthalazin-1(2H)-one 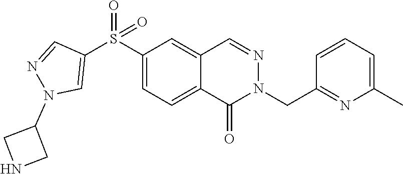 [SMILES: Cc1cccc(Cn2ncc3cc(S(=O)(=O)c4cnn(C5CNC5)c4)ccc3c2=O)n1] | MS (ES⁺): 437 (M + H)⁺ ¹H NMR (400 MHz, DMSO-d6) δ 8.71 (s, 1H), 8.64 (d, J = 1.8 Hz, 2H), 8.44 (d, J = 8.4 Hz, 1H), 8.33 (dd, J = 8.4, 1.9 Hz, 1H), 8.13 (s, 1H), 7.59 (t, J = 7.7 Hz, 1H), 7.13 (d, J = 7.6 Hz, 1H), 6.94 (d, J = 7.8 Hz, 1H), 5.39 (s, 2H), 5.24 (p, J = 7.2 Hz, 1H), 3.86 (t, J = 7.6 Hz, 2H), 3.71 (t, J = 7.9 Hz, 2H), 2.40 (s, 3H). (exchangeble NH not observed). |
| 277 | 6-((1-(2-aminoethyl)-1H-pyrazol-4-yl)sulfonyl)-2-((6- methylpyridin-2-yl)methyl)phthalazin-1(2H)-one 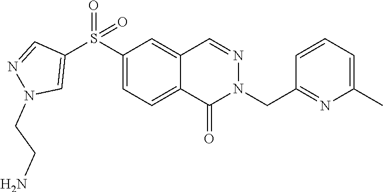 [SMILES: Cc1cccc(Cn2ncc3cc(S(=O)(=O)c4cnn(CCN)c4)ccc3c2=O)n1] | MS (ES⁺): 425 (M + H)⁺ ¹H NMR (400 MHz, DMSO-d6) δ 8.64 (s, 1H), 8.63 (d, J = 1.9 Hz, 1H), 8.57 (s, 1H), 8.44 (d, J = 8.4 Hz, 1H), 8.32 (dd, J = 8.4, 1.9 Hz, 1H), 8.04 (s, 1H), 7.59 (t, J = 7.7 Hz, 1H), 7.13 (d, J = 7.7 Hz, 1H), 6.95 (d, J = 7.8 Hz, 1H), 5.39 (s, 2H), 4.10 (t, J = 6.1 Hz, 2H), 2.90 (t, J = 6.1 Hz, 2H), 2.40 (s, 3H), 1.51 (s, 2H). |
| 278 (single enantiomer of 274) | (S)-2-((6-(1-hydroxyethyl)pyridin-2-yl)methyl)-6-((1-methyl-1H- pyrazol-4-yl)sulfonyl)phthalazin-1(2H)-one 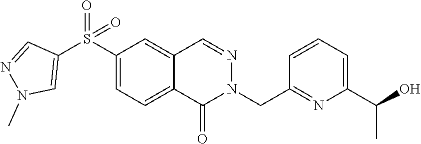 [SMILES: C[C@H](O)c1cccc(Cn2ncc3cc(S(=O)(=O)c4cnn(C)c4)ccc3c2=O)n1] | stereochemistry arbitrarily assigned (RT: 4.00 min.; 96.8% ee) MS (ES⁺): 426 (M + H)⁺ ¹H NMR (400 MHz, DMSO-d6) δ 8.65 (s, 1H), 8.63 (d, J = 1.9 Hz, 1H), 8.57 (s, 1H), 8.44 (d, J = 8.4 Hz, 1H), 8.31 (dd, J = 8.5, 1.9 Hz, 1H), 8.03 (d, J = 0.8 Hz, 1H), 7.69 (t, J = 7.8 Hz, 1H), 7.38 (d, J = 7.8 Hz, 1H), 6.99 (d, J = 7.7 Hz, 1H), 5.41 (s, 2H), 5.30 (d, J = 4.6 Hz, 1H), 4.66-4.59 (m, 1H), 3.88 (s, 3H), 1.28 (d, J = 6.6 Hz, 3H). |
| 279 (single enantiomer of 274) | (R)-2-((6-(1-hydroxyethyl)pyridin-2-yl)methyl)-6-((1-methyl-1H- pyrazol-4-yl)sulfonyl)phthalazin-1(2H)-one 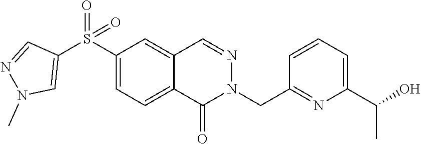 [SMILES: C[C@@H](O)c1cccc(Cn2ncc3cc(S(=O)(=O)c4cnn(C)c4)ccc3c2=O)n1] | stereochemistry arbitrarily assigned (RT: 4.27 min.; 96.6% ee) MS (ES⁺): 426 (M + H)⁺ ¹H NMR (400 MHz, DMSO-d6) δ 8.65 (s, 1H), 8.63 (d, J = 1.9 Hz, 1H), 8.57 (s, 1H), 8.44 (d, J = 8.4 Hz, 1H), 8.31 (dd, J = 8.5, 1.9 Hz, 1H), 8.03 (d, J = 0.8 Hz, 1H), 7.69 (t, J = 7.8 Hz, 1H), 7.38 (d, J = 7.8 Hz, 1H), 6.99 (d, J = 7.7 Hz, 1H), 5.41 (s, 2H), 5.30 (d, J = 4.6 Hz, 1H), 4.66-4.59 (m, 1H), 3.88 (s, 3H), 1.28 (d, J = 6.6 Hz, 3H). |
| 280 | 6-((1-(2-hydroxyethyl)-1H-pyrazol-3-yl)sulfonyl)-2-((6- methoxypyridin-3-yl)methyl)phthalazin-1(2H)-one 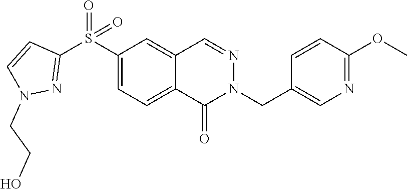 [SMILES: COc1ccc(Cn2ncc3cc(S(=O)(=O)c4ccn(CCO)n4)ccc3c2=O)cn1] | MS (ES⁺): 442 (M + H)⁺ ¹H NMR (400 MHz, DMSO-d6) δ 8.67 (s, 1H), 8.64 (d, J = 1.8 Hz, 1H), 8.46 (d, J = 8.4 Hz, 1H), 8.29 (dd, J = 8.4, 1.8 Hz, 1H), 8.20 (d, J = 2.4 Hz, 1H), 7.98 (d, J = 2.4 Hz, 1H), 7.69 (dd, J = 8.5, 2.5 Hz, 1H), 6.94 (d, J = 2.4 Hz, 1H), 6.81-6.74 (m, 1H), 5.28 (s, 2H), 4.95 (t, J = 5.2 Hz, 1H), 4.21 (t, J = 5.3 Hz, 2H), 3.81 (s, 3H), 3.70 (q, J = 5.3 Hz, 2H). |
| 281 | 2-((1H-pyrazol-3-yl)methyl)-6-((1-(2-methoxyethyl)-1H-pyrazol-4- yl)sulfonyl)phthalazin-1(2H)-one 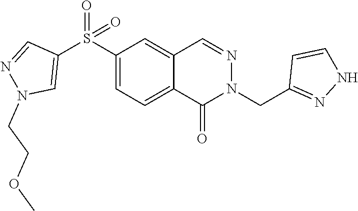 [SMILES: COCCn1cc(S(=O)(=O)c2ccc3c(=O)n(Cc4cc[nH]n4)ncc3c2)cn1] | MS (ES⁺): 415 (M + H)⁺ ¹H NMR (400 MHz, DMSO-d6) δ 12.67 (s, 1H), 8.66-8.59 (m, 2H), 8.57 (d, J = 0.7 Hz, 1H), 8.45 (d, J = 8.5 Hz, 1H), 8.31 (d, J = 8.4 Hz, 1H), 8.06 (d, J = 0.7 Hz, 1H), 7.63 (s, 1H), 6.14 (s, 1H), 5.31 (s, 2H), 4.31 (t, J = 5.2 Hz, 2H), 3.69 (t, J = 5.2 Hz, 2H), 3.20 (s, 3H). |
| 282 | 6-((2-(1-hydroxyethyl)thiazol-4-yl)sulfonyl)-2-((6-methylpyridin- 2-yl)methyl)phthalazin-1(2H)-one 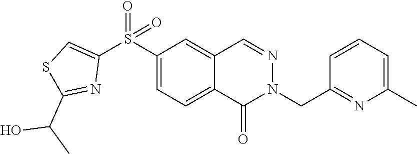 [SMILES: Cc1cccc(Cn2ncc3cc(S(=O)(=O)c4csc(C(C)O)n4)ccc3c2=O)n1] | MS (ES⁺): 443 (M + H)⁺ ¹H NMR (400 MHz, DMSO-d6) δ 8.77 (s, 1H), 8.72-8.67 (m, 2H), 8.48 (d, J = 8.5 Hz, 1H), 8.32 (dd, J = 8.4, 1.8 Hz, 1H), 7.59 (dd, J = 7.7, 7.7 Hz, 1H), 7.13 (d, J = 7.6 Hz, 1H), 6.97 (d, J = 7.8 Hz, 1H), 6.39 (d, J = 5.1 Hz, 1H), 5.39 (s, 2H), 4.95- 4.84 (m, 1H), 2.40 (s, 3H), 1.38 (d, J = 6.6 Hz, 3H). |
| 283 | 2-((6-(3-hydroxyoxetan-3-yl)pyridin-2-yl)methyl)-6-((1- methyl-1H-pyrazol-4-yl)sulfonyl)phthalazin-1(2H)-one 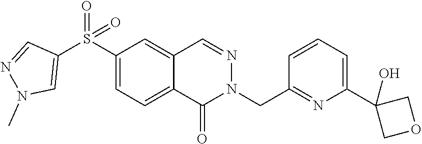 [SMILES: Cn1cc(S(=O)(=O)c2ccc3c(=O)n(Cc4cccc(C5(O)COC5)n4)ncc3c2)cn1] | MS (ES⁺): 454 (M + H)⁺ ¹H NMR (400 MHz, DMSO-d6) δ 8.66 (s, 1H), 8.63 (d, J = 1.8 Hz, 1H), 8.57 (s, 1H), 8.45 (d, J = 8.5 Hz, 1H), 8.31 (dd, J = 8.4, 1.9 Hz, 1H), 8.03 (s, 1H), 7.75 (t, J = 7.8 Hz, 1H), 7.47 (d, J = 7.8 Hz, 1H), 7.12 (d, J = 7.7 Hz, 1H), 6.46 (s, 1H), 5.51 (s, 2H), 4.78 (d, J = 6.1 Hz, 2H), 4.55 (d, J = 6.0 Hz, 2H), 3.88 (s, 3H). |
| 284 | 6-((1H-pyrazol-4-yl)sulfonyl)-2-((2-methoxypyridin-3- yl)methyl)phthalazin-1(2H)-one 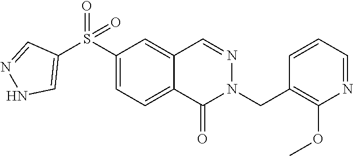 [SMILES: COc1ncccc1Cn1ncc2cc(S(=O)(=O)c3cn[nH]c3)ccc2c1=O] | MS (ES⁺): 398.2 (M + H)⁺ ¹H NMR (400 MHz, DMSO-d6) δ: 8.65 (d, J = 3.2 Hz, 2H), 8.47 (d, J = 8.4 Hz, 1H), 8.35-8.33 (m, 3H), 8.10 (dd, J = 5.2 Hz, 2.0 Hz, 1H), 7.36-7.34 (m, 1H), 6.90 (dd, J = 7.2 Hz, 5.6 Hz, 1H), 5.30 (s, 2H), 3.92 (s, 3H). |
| 285 | 2-((1-(2-hydroxyethyl)-1H-pyrazol-3-yl)methyl)-6-((6- methoxypyridin-3-yl)sulfonyl)phthalazin-1(2H)-one 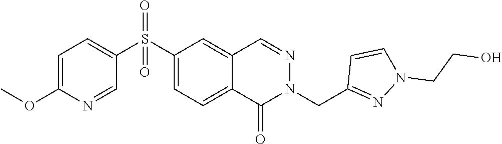 [SMILES: COc1ccc(S(=O)(=O)c2ccc3c(=O)n(Cc4ccn(CCO)n4)ncc3c2)cn1] | MS (ES⁺): 442 (M + H)⁺ ¹H NMR (400 MHz, MeOD) δ 8.84 (d, J = 2.6 Hz, 1H), 8.59 (d, J = 1.8 Hz, 1H), 8.51 (d, J = 8.5 Hz, 1H), 8.47 (s, 1H), 8.33 (dd, J = 8.4, 1.8 Hz, 1H), 8.19 (dd, J = 8.9, 2.6 Hz, 1H), 7.55 (d, J = 2.3 Hz, 1H), 6.93 (dd, J = 8.9, 0.7 Hz, 1H), 6.24 (d, J = 2.3 Hz, 1H), 5.39 (s, 2H), 4.15 (t, J = 5.3 Hz, 2H), 3.99 (s, 3H), 3.82 (t, J = 5.4 Hz, 2H). |
| 286 | 2-((1H-pyrazol-3-yl)methyl)-6-((6-methoxypyridin-2- yl)sulfonyl)phthalazin-1(2H)-one 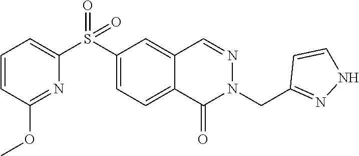 [SMILES: COc1cccc(S(=O)(=O)c2ccc3c(=O)n(Cc4cc[nH]n4)ncc3c2)n1] | MS (ES⁺): 398 (M + H)⁺ ¹H NMR (DMSO-d6) δ: 12.66 (s, 1H), 8.69 (d, J = 1.8 Hz, 1H), 8.66 (s, 1H), 8.49 (d, J = 8.4 Hz, 1H), 8.38 (dd, J = 8.4, 1.8 Hz, 1H), 8.04 (dd, J = 8.4, 7.3 Hz, 1H), 7.85 (dd, J = 7.3, 0.7 Hz, 1H), 7.64-7.60 (m, 1H), 7.14 (dd, J = 8.4, 0.7 Hz, 1H), 6.15 (d, J = 2.2 Hz, 1H), 5.31 (s, 2H), 3.77 (s, 3H). |
| 287 | 6-((1H-pyrazol-4-yl)sulfonyl)-2-((6-cyclopropylpyridin-2- yl)methyl)phthalazin-1(2H)-one 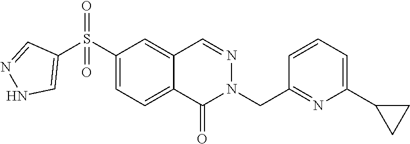 [SMILES: O=c1c2ccc(S(=O)(=O)c3cn[nH]c3)cc2cnn1Cc1cccc(C2CC2)n1] | MS (ES⁺): 408 (M + H)⁺ ¹H NMR (DMSO-d6) δ: 13.92 (s, 1H), 8.69-8.53 (m, 1H), 8.63 (s, 2H), 8.43 (d, J = 8.4 Hz, 1H), 8.32 (dd, J = 8.4, 1.9 Hz, 1H), 8.14-7.97 (m, 1H), 7.54 (dd, J = 7.7, 7.7 Hz, 1H), 7.15-7.11 (d, J = 7.7 Hz, 1H), 6.90 (d, J = 7.7 Hz, 1H), 5.35 (s, 2H), 2.05-1.94 (m, 1H), 0.86-0.77 (m, 2H), 0.72-0.64 (m, 2H). |
| 288 | 2-((1H-pyrazol-3-yl)methyl)-6-((6-methylpyridin-2- yl)sulfonyl)phthalazin-1(2H)-one 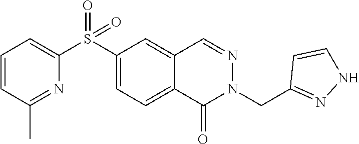 [SMILES: Cc1cccc(S(=O)(=O)c2ccc3c(=O)n(Cc4cc[nH]n4)ncc3c2)n1] | MS (ES⁺): 382 (M + H)⁺ ¹H NMR (400 MHz, DMSO-d6) δ 12.65 (s, 1H), 8.68 (s, 1H), 8.65 (s, 1H), 8.47 (d, J = 8.5 Hz, 1H), 8.35-8.28 (m, 1H), 8.13- 8.04 (m, 2H), 7.62 (s, 1H), 7.58 (dd, J = 7.3, 1.5 Hz, 1H), 6.14 (s, 1H), 5.30 (s, 2H), 2.47 (s, 3H). |
| 289 | 2-((1H-pyrazol-3-yl)methyl)-6-((5-methylpyridin-2- yl)sulfonyl)phthalazin-1(2H)-one 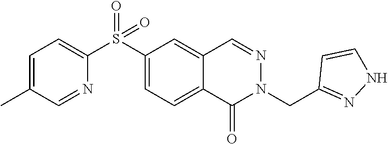 [SMILES: Cc1ccc(S(=O)(=O)c2ccc3c(=O)n(Cc4cc[nH]n4)ncc3c2)nc1] | MS (ES⁺): 382.3 (M + H)⁺ ¹H NMR (400 MHz, DMSO-d6) δ: 8.67 (d, J = 1.6 Hz, 1H), 8.65 (s, 1H), 8.55 (d, J = 1.6 Hz, 1H), 8.47 (d, J = 8.4 Hz, 1H), 8.30 (dd, J = 8.4 Hz, 2.0 Hz, 1H), 8.20 (d, J = 8.0 Hz, 1H), 8.00 (dd, J = 8.0 Hz, 1.2 Hz, 1H), 7.58 (d, J = 1.6 Hz, 1H), 6.15 (d, J = 2.4 Hz, 1H), 5.32 (s, 2H), 2.38 (s, 3H). |
| 290 | 2-((1H-pyrazol-3-yl)methyl)-6-((6-methylpyridin-3- yl)sulfonyl)phthalazin-1(2H)-one 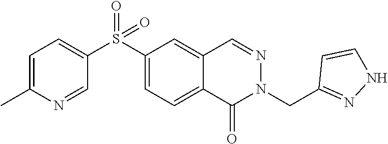 [SMILES: Cc1ccc(S(=O)(=O)c2ccc3c(=O)n(Cc4cc[nH]n4)ncc3c2)cn1] | MS (ES⁺): 382.31 (M + H)⁺ ¹H NMR (400 MHz, DMSO-d6) δ: 12.69 (s, 1H), 9.07 (d, J = 2.4 Hz, 1H), 8.72 (d, J = 1.6 Hz, 1H), 8.59 (s, 1H), 8.44 (d, J = 8.4 Hz, 1H), 8.36 (dd, J = 8.4 Hz, 2.0 Hz, 1H), 8.30 (dd, J = 8.4 Hz, 1.4 Hz, 1H), 7.58- 7.54 (m, 2H), 6.13 (d, J = 2.0 Hz, 1H), 5.31-5.28 (m, 2H), 2.65 (s, 3H). |
| 291 | 6-(1H-pyrazol-4-ylsulfonyl)-2-((5-methoxy-6-methylpyridin-2- yl)methyl)phthalazin-1(2H)-one 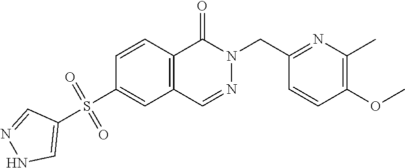 [SMILES: COc1ccc(Cn2ncc3cc(S(=O)(=O)c4cn[nH]c4)ccc3c2=O)nc1C] | MS (ES⁺): 412.1 (M + H)⁺ ¹H NMR (400 MHz, DMSO-d6) δ: 13.92 (s, 1H), 8.62 (s, 2H), 8.45-8.42 (m, 1.6H), 8.33-8.30 (m, 2.2H), 7.24 (d, J = 8.8 Hz, 1H), 7.02 (d, J = 8.4 Hz, 1H), 5.33 (s, 2H), 3.77 (s, 3H), 2.29 (s, 3H). |
| 292 | 2-((1-(2-hydroxyethyl)-1H-pyrazol-3-yl)methyl)-6-((5- methoxypyridin-2-yl)sulfonyl)phthalazin-1(2H)-one 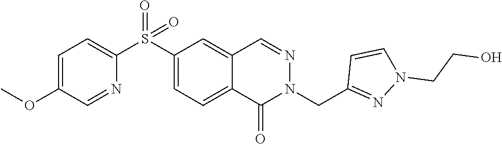 [SMILES: COc1ccc(S(=O)(=O)c2ccc3c(=O)n(Cc4ccn(CCO)n4)ncc3c2)nc1] | MS (ES⁺): 442.3 (M + H)⁺ ¹H NMR (400 MHz, DMSO-d6) δ: 8.64 (d, J = 2.4 Hz, 2H), 8.46 (d, J = 8.4 Hz, 1H), 8.40 (d, J = 2.0 Hz, 1H), 8.29-8.25 (m, 2H), 7.68 (dd, J = 8.8 Hz, 2.8 Hz, 1H), 7.58 (d, J = 2.4 Hz, 1H), 6.09 (d, J = 2.4 Hz, 1H), 5.27 (s, 2H), 4.06 (t, J = 5.6 Hz, 2H), 3.91 (s, 3H), 3.67 (t, J = 5.6 Hz, 2H). |
| 293 | 6-(1H-pyrazol-4-ylsulfonyl)-2-((2,3-dihydrofuro[2,3-b]pyridin-5- yl)methyl)phthalazin-1(2H)-one 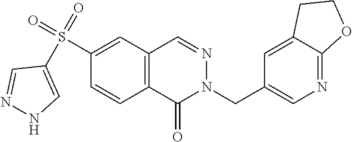 [SMILES: O=c1c2ccc(S(=O)(=O)c3cn[nH]c3)cc2cnn1Cc1cnc2c(c1)CCO2] | MS (ES⁺): 410.1 (M + H)⁺ ¹H NMR (400 MHz, DMSO-d6) δ: 13.91 (s, 1H), 8.62-8.59 (m, 3H), 8.45-8.42 (m, 1H), 8.33-8.29 (m, 2H), 8.04 (br, 1H), 7.94 (s, 1H), 7.59 (s, 1H), 5.24 (s, 2H), 4.52 (t, J = 8.4 Hz, 2H), 3.16 (t, J = 8.4 Hz, 2H). |
| 294 | 6-((1H-pyrazol-4-yl)sulfonyl)-2-((6-methoxypyridin-2- yl)methyl)phthalazin-1(2H)-one 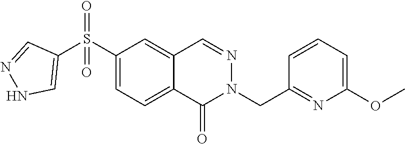 [SMILES: COc1cccc(Cn2ncc3cc(S(=O)(=O)c4cn[nH]c4)ccc3c2=O)n1] | MS (ES⁺): 398 (M + H)⁺ ¹H NMR (DMSO-d6) δ: 13.92 (s, 1H), 8.65 (s, 1H), 8.63 (d, J = 1.8 Hz, 1H), 8.44 (d, J = 8.4 Hz, 1H), 8.38-8.28 (m, 2H), 8.32 (dd, J = 8.4, 1.9 Hz, 1H), 7.61 (dd, J = 8.2, 7.3 Hz, 1H), 6.74 (d, J = 7.3 Hz, 1H), 6.68 (d, J = 8.2 Hz, 1H), 5.36 (s, 2H), 3.69 (s, 3H). |
| 295 | 6-((1H-pyrazol-4-yl)sulfonyl)-2-((6-methylpyridin-3- yl)methyl)phthalazin-1(2H)-one 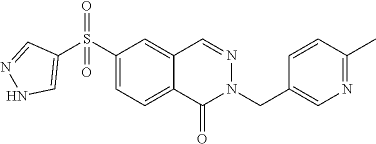 [SMILES: Cc1ccc(Cn2ncc3cc(S(=O)(=O)c4cn[nH]c4)ccc3c2=O)cn1] | MS (ES⁺): 382 (M + H)⁺ ¹H NMR (DMSO-d6) δ: 13.91 (s, 1H), 8.62 (s, 1H), 8.60 (d, J = 1.8 Hz, 1H), 8.45 (d, J = 2.3 Hz, 1H), 8.43 (d, J = 8.4 Hz, 1H), 8.36-8.26 (m, 2H), 8.30 (dd, J = 8.4, 1.9 Hz, 1H), 7.61 (dd, J = 8.0, 2.4 Hz, 1H), 7.19 (d, J = 8.0 Hz, 1H), 5.31 (s, 2H), 2.42 (s, 3H). |
| 296 | (R)-6-((2-(1-hydroxyethyl)thiazol-4-yl)sulfonyl)-2-((6- methylpyridin-2-yl)methyl)phthalazin-1(2H)-one 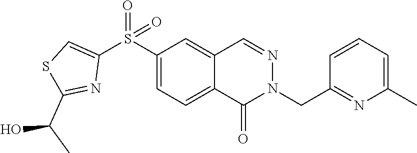 [SMILES: Cc1cccc(Cn2ncc3cc(S(=O)(=O)c4csc([C@@H](C)O)n4)ccc3c2=O)n1] | stereochemistry arbitrarily assigned (RT: 3.08 min.; 96.7% ee) |
| 297 | (S)-6-((2-(1-hydroxyethyl)thiazol-4-yl)sulfonyl)-2-((6- methylpyridin-2-yl)methyl)phthalazin-1(2H)-one 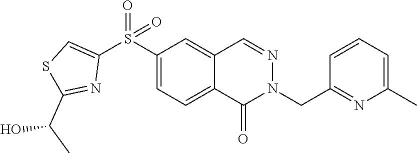 [SMILES: Cc1cccc(Cn2ncc3cc(S(=O)(=O)c4csc([C@H](C)O)n4)ccc3c2=O)n1] | stereochemistry arbitrarily assigned (RT: 3.49 min.; 95.5% ee) |

### Example 298—6-((1H-pyrazol-4-yl)sulfonyl)-2-(furo[3,2-b]pyridin-5-ylmethyl)phthalazin-1(2H)-one

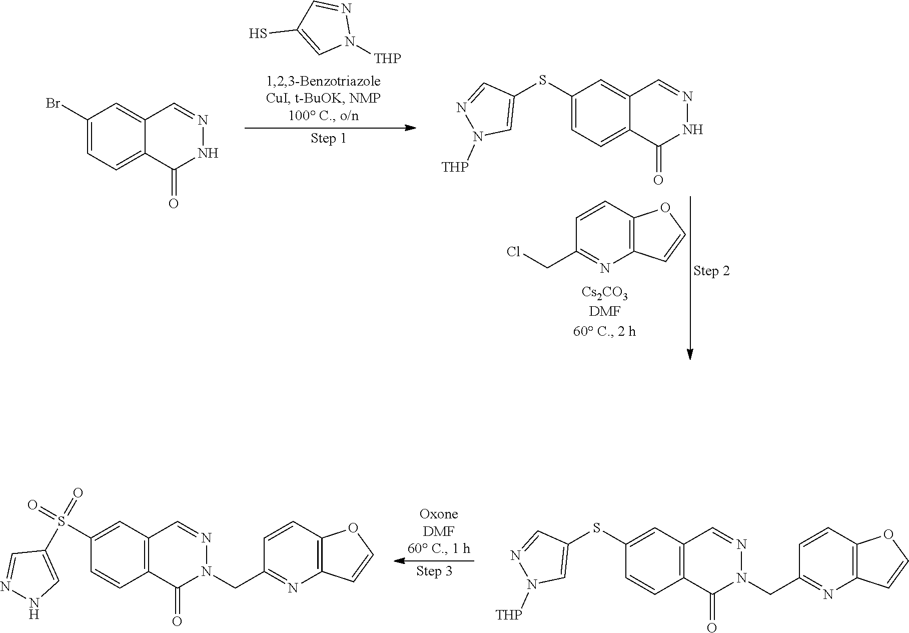

### Step 1

A mixture of 1-(tetrahydro-2H-pyran-2-yl)-1H-pyrazole-4-thiol (Intermediate 62, 62 g, 337 mmol), 6-bromophthalazin-1(2H)-one (54 g, 241 mmol), CuI (6.4 g, 33.7 mmol) and 1,2,3-Benztriazole (8 g, 67.4 mmol) in NMP (2 L) was added t-BuOK (54 g, 482 mmol) at room temperature. The reaction was stirred at 100° C. overnight under nitrogen atmosphere. After LCMS indicated the reaction was completed, the reaction mixture was added to the water (10 L), stirred at room temperature for 3 hours and filtered. The residue was lyophilized to give 6-((1-(tetrahydro-2H-pyran-2-yl)-1H-pyrazol-4-yl)thio)phthalazin-1(2H)-one (75 g, 98% purity) as a yellow solid. MS (ES+): 329.1 (M+H)+

### Step 2

To the solution of 6-((1-(tetrahydro-2H-pyran-2-yl)-1H-pyrazol-4-yl)thio)phthalazin-1(2H)-one (197 mg, 0.60 mmol), Cs₂CO₃ (390 mg, 1.20 mmol) in DMF (10 mL) was added 5-(chloromethyl)furo[3,2-b]pyridine (Intermediate 62), 100 mg, 0.60 mmol) stirred at 60° C. for 2 hours. After LCMS indicated the reaction was completed, the mixture was extracted with DCM (10 mL×3). The organic layer was washed with brine, dried over Na₂SO₄, filtered and concentrated at 30° C. under reduced pressure. The residue was purified by flash column chromatography (12 g, petroleum ether/ethyl acetate=100:00/30:70) to give 2-(furo[3,2-b]pyridin-5-ylmethyl)-6-((1-(tetrahydro-2H-pyran-2-yl)-1H-pyrazol-4-yl)thio)phthalazin-1 (2H)-one (150 mg, 93.77% purity) as a yellow solid. MS (ES+): 460.1 (M+H)+

### Step 3

To the solution of 2-(furo[3,2-b]pyridin-5-ylmethyl)-6-((1-(tetrahydro-2H-pyran-2-yl)-1H-pyrazol-4-yl)thio)phthalazin-1(2H)-one (150 mg, 0.33 mmol), OXONE (509 mg, 0.83 mmol) in DMF (10 mL) stirred at 60° C. for 1 hours. After LCMS indicated the reaction was completed, the mixture was extracted with DCM (10 mL×3). The organic layer was washed with brine, dried over Na₂SO₄, filtered and concentrated at 30° C. under reduced pressure the residue was purified by prep-H PLC (Column: Waters X-SELECT C18 OBD 10 μm 19*250 mm; Flow Rate: 20 mL/min; solvent system: MeCN/(10 mmol/L NH₄HCO₃/water) gradient: MeCN: 40%-95%; collection wavelength: 214 nm). The fractions were concentrated at 42° C. under reduced pressure to remove MeCN, and the residue was lyophilized to give the title compound (53.66 mg, 100.00% purity) as white solid. MS (ES+): 408.1 (M+H)⁺. ¹H NMR (400 MHz, DMSO-d₆+CF3CO2D) δ: 8.65-8.64 (m, 2H), 8.45 (d, J=8.4 Hz, 1H), 8.36-8.31 (m, 4H), 8.05 (d, J=8.4 Hz, 1H), 7.32 (d, J=8.4 Hz, 1H), 7.09 (s, 1H), 5.58 (s, 2H).

### Example 299—6-((1H-pyrazol-3-yl)sulfonyl)-2-((2,3-dihydrofuro[3,2-b]pyridin-5-yl)methyl)phthalazin-1(2H)-one

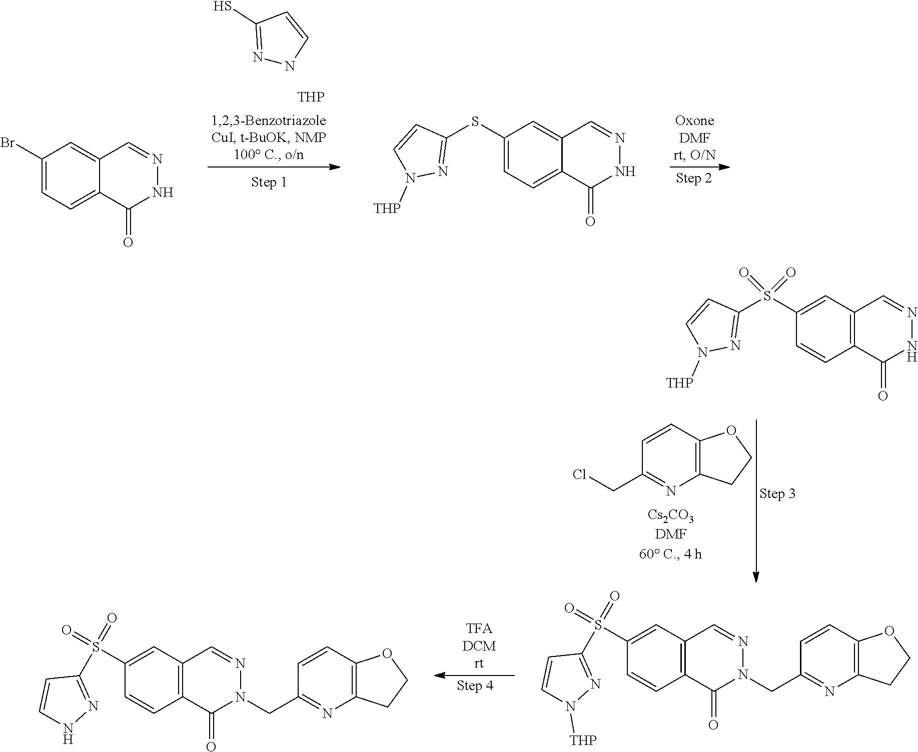

### Step 1

A mixture of 1-(tetrahydro-2H-pyran-2-yl)-1H-pyrazole-3-thiol (Intermediate 63, 2.8 g, 15.2 mmol), 6-bromophthalazin-1(2H)-one (2.9 g, 12.9 mmol), CuI (246 mg, 1.29 mmol) and 1,2,3-Benztriazole (307 mg, 2.58 mmol) in DMF (30 mL) was added t-BuOK (3.4 g, 30.4 mmol) at room temperature. The reaction was stirred at 100° C. overnight under nitrogen atmosphere. After LCMS indicated the reaction was completed, the reaction mixture was added to the water (200 mL), the solid was filtered and dried to give 6-((1-(tetrahydro-2H-pyran-2-yl)-1H-pyrazol-3-yl)thio)phthalazin-1(2H)-one (2.4 g, 72.75% purity) as a yellow solid. MS (ES+): 329.1 (M+H)+

### Step 2

A solution of 6-((1-(tetrahydro-2H-pyran-2-yl)-1H-pyrazol-3-yl)thio)phthalazin-1(2H)-one (1.7 g, 5.18 mmol) in DMF (15 mL) was added OXONE (6.37 g, 10.36 mmol) was stirred at room temperature overnight. After LCMS indicated the reaction was completed, the reaction mixture was diluted with sat. aq NaHCO₃ (40 mL) and extracted with EtOAc (50 mL×4). The organic layers were washed with sat. aq. NH₄Cl (50 mL×2), dried over Na₂SO₄ and concentrated at 40° C. under reduced pressure to give 6-((1-(tetrahydro-2H-pyran-2-yl)-1H-pyrazol-3-yl)sulfonyl)phthalazin-1(2H)-one (2.0 g, 77% purity) as a yellow solid. MS (ES+): 361.2 (M+H)+

### Step 3

A mixture of 5-(chloromethyl)-2,3-dihydrofuro[3,2-b]pyridine (Intermediate 64, 320 mg, 1.89 mmol), Cs₂CO₃ (1.95 g, 6.0 mmol) and 6-((1-(tetrahydro-2H-pyran-2-yl)-1H-pyrazol-3-yl)sulfonyl)phthalazin-1(2H)-one (328 mg, 1.0 mmol) in DMF (4 mL) was stirred at 60° C. for 4 hours. After LCMS indicated the reaction was completed, the reaction mixture was diluted with water (25 mL) and extracted with EtOAc (50 mL×3). The combined organic layers were washed with saturated ammonium chloride solution (40 mL×2), dried over Na₂SO₄, filtered, and concentrated at 40° C. under reduced pressure. The residual solid was purified by flash column chromatography (12 g, petroleum ether/ethyl acetate=100:00˜60:40) to give 2-((2,3-dihydrofuro[3,2-b]pyridin-5-yl)methyl)-6-((1-(tetrahydro-2H-pyran-2-yl)-1H-pyrazol-3-yl)sulfonyl)phthalazin-1(2H)-one (600 mg, 100% purity) as a yellow solid. MS (ES+): 494.2 (M+H)+

### Step 4

To a solution of 2-((2,3-dihydrofuro[3,2-b]pyridin-5-yl)methyl)-6-((1-(tetrahydro-2H-pyran-2-yl)-1H-pyrazol-3-yl)sulfonyl)phthalazin-1(2H)-one (600 mg, 1.22 mol) in DCM (2 mL) was added TFA (6 mL) and the mixture was stirred at room temperature for 1 hour. After LCMS indicated the reaction to be complete, the mixture was concentrated under reduced pressure. The residue was diluted with H₂O (15 mL) and adjusted pH to 8 with K₂CO₃. A solid precipitated and the suspension was filtered. The filtered cake was purified by prep-HPLC (Column: Waters Xbridge Prep C18 OBD 10 μm 19*250 mm; Flow Rate: 20 mL/min; solvent system: MeCN/(10 mmol/L NH₄HCO₃/water) gradient: MeCN: 25%-95%; collection wavelength: 214 nm). The fractions were concentrated at 40° C. under reduced pressure to remove MeCN, and the residue was lyophilized to give the desired compound (252.69 mg, 100% purity) as white solid. MS (ES+): 410.3 (M+H)+ ¹H NMR (400 MHz, DMSO-d6) δ: 13.99 (br, 1H), 8.66 (s, 1H), 8.64 (d, J=1.6 Hz, 1H), 8.46 (d, J=8.4 Hz, 1H), 8.29 (dd, J=8.4 Hz, 1.6 Hz, 1H), 8.04 (t, J=1.6 Hz, 1H), 7.05 (d, J=8.0 Hz, 1H), 6.99-0.96 (m, 2H), 5.34 (s, 2H), 4.59 (t, J=8.8 Hz, 2H), 3.18 (t, J=8.8 Hz, 2H).

### Example 300—7-((1H-pyrazol-4-yl)sulfonyl)-3-((5-methyl-1H-pyrazol-3-yl)methyl)pyrido[3,4-d]pyridazin-4(3H)-one

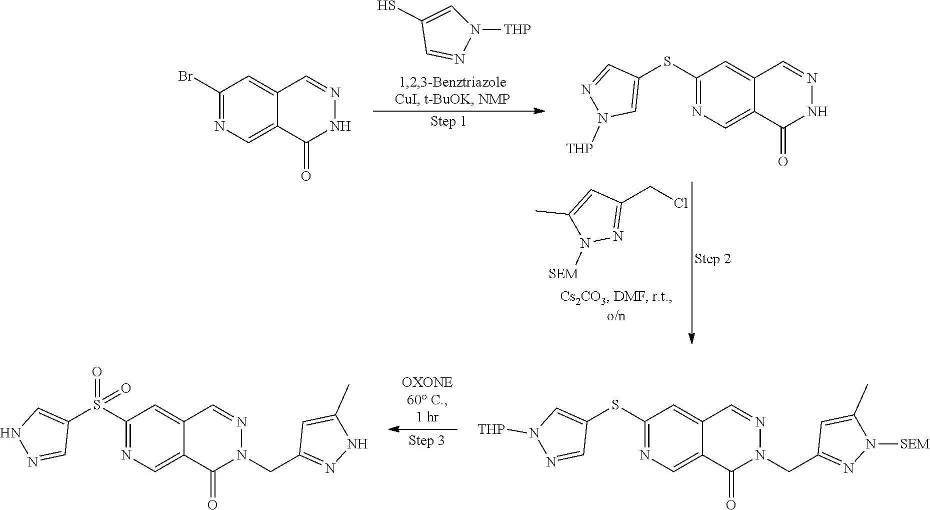

### Step 1

A mixture of 1-(tetrahydro-2H-pyran-2-yl)-1H-pyrazole-4-thiol (Intermediate 59, 850 mg, 4.6 mmol), 7-bromopyrido[3,4-d]pyridazin-4(3H)-one (739 mg, 3.3 mmol), CuI (125 mg, 0.66 mmol) and 1,2,3-Benztriazole (157 mg, 1.32 mmol) in NMP (10 mL) was added t-BuOK (784 mg, 7 mmol) at room temperature. The reaction was stirred at 100° C. overnight under nitrogen atmosphere. After LCMS indicated the reaction was completed, the reaction mixture was added to the water (50 mL) and extracted with EtOAc (10 mL×6). The organic layers were concentrated at 45° C. under reduced pressure. The residue was purified by flash column chromatography (20 g, DCM/MeOH=100:00˜90:10) to give 7-((1-(tetrahydro-2H-pyran-2-yl)-1H-pyrazol-4-yl)thio)pyrido[3,4-d]pyridazin-4(3H)-one (0.9 g, 82.29% purity) as a yellow solid. MS (ES+): 330.3 (M+H)+

### Step 2

3-(chloromethyl)-5-methyl-1-((2-(trimethylsilyl)ethoxy)methyl)-1H-pyrazole (Intermediate 65, 1.3 mmol) was dissolved in DMF (4 mL) and CS₂CO₃ (1.95 g, 6.0 mmol) and 7-((1-(tetrahydro-2H-pyran-2-yl)-1H-pyrazol-4-yl)thio)pyrido[3,4-d]pyridazin-4(3H)-one (330 mg, 1.0 mmol) were added at room temperature; the reaction mixture was stirred at room temperature overnight. After LCMS indicated the reaction was completed, the reaction mixture was diluted with water (15 mL) and extracted with EtOAc (50 mL×3). The combined organic layers were washed with saturated ammonium chloride solution (40 mL×2), dried over Na₂SO₄, filtered and concentrated at 40° C. under reduced pressure. The residual solid was purified by flash column chromatography (12 g, petroleum ether/ethyl actate=100:00˜70:30) to give 3-((5-methyl-1-((2-(trimethylsilyl)ethoxy)methyl)-1H-pyrazol-3-yl)methyl)-7-((1-(tetrahydro-2H-pyran-2-yl)-1H-pyrazol-4-yl)thio)pyrido[3,4-d]pyridazin-4(3H)-one (200 mg, 85% purity) as a yellow solid. MS (ES+): 554.3 (M+H)+

### Step 3

A solution of 3-((5-methyl-1-((2-(trimethylsilyl)ethoxy)methyl)-1H-pyrazol-3-yl)methyl)-7-((1-(tetrahydro-2H-pyran-2-yl)-1H-pyrazol-4-yl)thio)pyrido[3,4-d]pyridazin-4(3H)-one (200 mg, 0.36 mmol) in DMF (3 mL) was added OXONE (885 mg, 1.44 mmol) was stirred at 60° C. for 1 hour. After LCMS indicated the reaction was completed, the reaction mixture was diluted with water (8 mL), neutralized with K₂CO₃ and extracted with EtOAc (10 mL×4). The organic layers were washed with sat. aq. NH₄Cl (20 mL×2), concentrated and purified by prep-HPLC (Column: Waters Xbridge Prep C18 OBD 10 μm 19*250 mm; Flow Rate: 20 mL/min; solvent system: MeCN/(10 mmol/L NH₄HCO₃/water) gradient: MeCN: 20%-95%; collection wavelength: 214 nm). The fractions were concentrated at 40° C. under reduced pressure to remove MeCN, and the residue was then lyophilized to give the desired compound (21.18 mg, 100% purity) as white solid. MS (ES+): 372.1 (M+H)+. ¹H NMR (400 MHz, DMSO-d6) δ: 13.93 (br, 1H), 12.34 (br, 1H), 9.51 (s, 1H), 8.69 (s, 1H), 8.66 (s, 1H), 8.33 (br, 2H), 5.88 (s, 1H), 5.31-5.24 (m, 2H), 2.14-2.07 (m, 3H).

The following compounds were made using similar procedures described above:

| Example | | |
|---|---|---|
| No. | Example Structure/Name | LCMS/¹H NMR data |
| 301 | 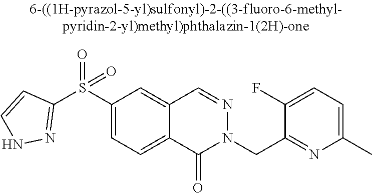 [SMILES: Cc1ccc(F)c(Cn2ncc3cc(S(=O)(=O)c4cc[nH]n4)ccc3c2=O)n1] | MS (ES+): 400 (M + H)+ 1H NMR (400 MHz, DMSO) δ 13.99 (s, 1H), 8.66-8.62 (m, 2H), 8.45 (d, J = 8.4 Hz, 1H), 8.29 (dd, J = 8.4, 1.8 Hz, 1H), 8.03 (d, J = 2.4 Hz, 1H), 7.59 (dd, J = 10.0, 8.4 Hz, 1H), 7.23 (dd, J = 8.5, 3.8 Hz, 1H), 6.97 (d, J = 2.5 Hz, 1H), 5.47 (d, J = 1.7 Hz, 2H), 2.30 (d, J = 1.0 Hz, 3H). |
| 302 | 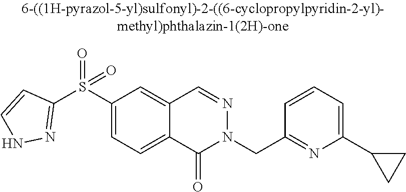 [SMILES: O=c1c2ccc(S(=O)(=O)c3cc[nH]n3)cc2cnn1Cc1cccc(C2CC2)n1] | MS (ES+): 408 (M + H)+ 1H NMR (400 MHz, DMSO) δ 14.00 (s, 1H), 8.68 (s, 1H), 8.67 (d, J = 1.8 Hz, 1H), 8.46 (d, J = 8.4 Hz, 1H), 8.30 (dd, J = 8.4, 1.9 Hz, 1H), 8.03 (d, J = 2.4 Hz, 1H), 7.58 (t, J = 7.7 Hz, 1H), 7.15 (d, J = 7.7 Hz, 1H), 6.97 (d, J = 2.4 Hz, 1H), 6.94 (d, J = 7.7 Hz, 1H), 5.37 (s, 2H), 2.02 (tt, J = 8.4, 4.7 Hz, 1H), 0.84 (dd, J = 8.0, |
| | | 3.1 Hz, 2H), 0.71 (dq, J = 6.7, |
| | | 3.6 Hz, 2H). |
| 303 | 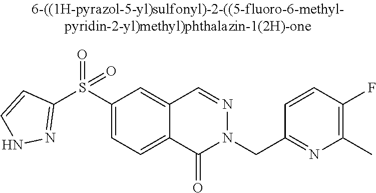 [SMILES: Cc1nc(Cn2ncc3cc(S(=O)(=O)c4cc[nH]n4)ccc3c2=O)ccc1F] | MS (ES+): 400 (M + H)+ 1H NMR (400 MHz, DMSO) δ 13.98 (s, 1H), 8.68 (s, 1H), 8.66 (d, J = 1.8 Hz, 1H), 8.46 (d, J = 8.4 Hz, 1H), 8.30 (dd, J = 8.4, 1.9 Hz, 1H), 8.03 (dd, J = 2.5, 1.5 Hz, 1H), 7.58-7.49 (m, 1H), 7.12 (dd, J = 8.5, 3.7 Hz, 1H), 6.97 (t, J = 2.1 Hz, 1H), 5.38 (s, 2H), 2.38 (d, J = 3.0 Hz, 3H). |
| 304 | 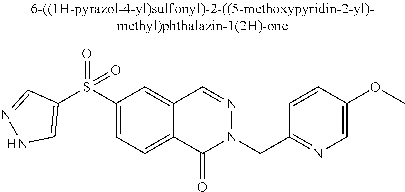 [SMILES: COc1ccc(Cn2ncc3cc(S(=O)(=O)c4cn[nH]c4)ccc3c2=O)nc1] | MS (ES+): 398 (M + H)+ 1H NMR (DMSO) δ: 13.91 (s, 1H), 8.64-8.59 (m, 2H), 8.57- 7.96 (s (br), 2H), 8.43 (d, J = 8.4 Hz, 1H), 8.31 (dd, J = 8.4, 1.9 Hz, 1H), 8.16 (d, J = 2.9 Hz, 1H), 7.33 (dd, J = 8.6, 3.0 Hz, 1H), 7.24 (d, J = 8.6 Hz, 1H), 5.38 (s, 2H), 3.78 (s, 3H). |
| 305 | 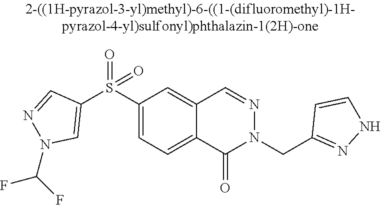 | MS (ES+): 407 (M + H)+ 1H NMR (DMSO) δ: 12.65 (s, 1H), 9.15 (s, 1H), 8.67 (d, J = 1.8 Hz, 1H), 8.61 (s, 1H), 8.47 (d, J = 8.5 Hz, 1H), 8.40 (s, 1H), 8.36 (dd, J = 8.5, 1.9 Hz, 1H), 7.85 (t, J = 58.3, 1H), 7.62 (s, 1H), 7.37 (s, 1H), 6.16-6.11 (m, 1H), 5.31 (s, 2H). |
| 306 | 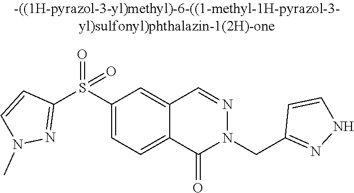 [SMILES: Cn1ccc(S(=O)(=O)c2ccc3c(=O)n(Cc4cc[nH]n4)ncc3c2)n1] | MS (ES+): 371 (M + H)+ 1H NMR (DMSO) δ: 12.65 (s, 1H), 8.66-8.60 (m, 2H), 8.46 (d, J = 8.4 Hz, 1H), 8.27 (dd, J = 8.5, 1.9 Hz, 1H), 7.97 (d, J = 2.4 Hz, 1H), 7.65-7.60 (m, 1H), 6.95 (d, J = 2.4 Hz, 1H), 6.13 (d, J = 2.1 Hz, 1H), 5.30 (s, 2H), 3.90 (s, 3H). |
| 307 | 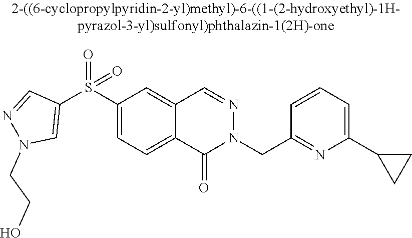 [SMILES: O=c1c2ccc(S(=O)(=O)c3cnn(CCO)c3)cc2cnn1Cc1cccc(C2CC2)n1] | MS (ES+): 452 (M + H)+ 1H NMR (400 MHz, DMSO) δ 8.64 (d, J = 2.0 Hz, 1H), 8.63 (s, 1H), 8.54 (d, J = 0.7 Hz, 1H), 8.44 (d, J = 8.4 Hz, 1H), 8.32 (dd, J = 8.4, 1.9 Hz, 1H), 8.06 (d, J = 0.8 Hz, 1H), 7.55 (t, J = 7.7 Hz, 1H), 7.13 (d, J = 7.7 Hz, 1H), 6.90 (d, J = 7.7 Hz, 1H), 5.35 (s, 2H), 4.95 (t, J = 5.3 Hz, 1H), 4.19 (t, J = 5.4 Hz, 2H), 3.73 (q, J = 5.3 Hz, 2H), 2.05-1.94 (m, 1H), 0.82 (dt, J = 8.2, 3.1 Hz, 2H), 0.73-0.65 (m, 2H). |
| 308 | 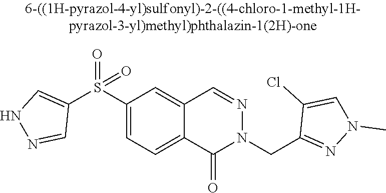 [SMILES: Cn1cc(Cl)c(Cn2ncc3cc(S(=O)(=O)c4cn[nH]c4)ccc3c2=O)n1] | MS (ES+): 405/407 (M + H)+ 1H NMR (DMSO) δ: 13.91 (s, 1H), 8.62 (s, 1H), 8.59 (d, J = 1.8 Hz, 1H), 8.58 (d, J = 0.7 Hz, 1H), 8.43 (d, J = 8.4 Hz, 1H), 8.30 (dd, J = 8.4, 1.8 Hz, 1H), 8.05 (s, 1H), 7.89 (s, 1H), 5.28 (s, 2H), 3.73 (s, 3H). |
| 309 | 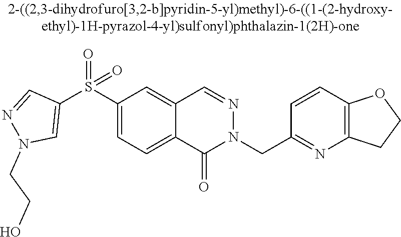 | MS (ES+): 454 (M + H)+ 1H NMR (400 MHz, DMSO) δ 8.62 (d, J = 2.5 Hz, 2H), 8.53 (s, 1H), 8.44 (d, J = 8.5 Hz, 1H), 8.31 (dd, J = 8.4, 1.8 Hz, 1H), 8.05 (d, J = 0.7 Hz, 1H), 7.05 (d, J = 8.3 Hz, 1H), 6.98 (d, J = 8.3 Hz, 1H), 5.34 (s, 2H), 4.94 (s, 1H), 4.59 (t, J = 8.8 Hz, 2H), 4.18 (t, J = 5.3 Hz, 2H), 3.74-3.70 (m, 2H), 3.18 (t, J = 8.8 Hz, 2H). |
| 310 | 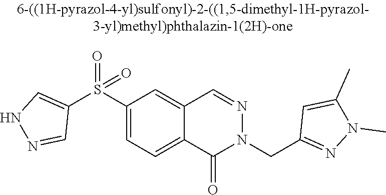 [SMILES: Cc1cc(Cn2ncc3cc(S(=O)(=O)c4cn[nH]c4)ccc3c2=O)nn1C] | MS (ES+): 385 (M + H)+ 1H NMR (DMSO) δ: 13.90 (s, 1H), 8.61 (s, 1H), 8.59 (d, J = 1.8 Hz, 1H), 8.58 (s, 1H), 8.42 (d, J = 8.4 Hz, 1H), 8.29 (dd, J = 8.4, 1.8 Hz, 1H), 8.04 (s, 1H), 5.88 (s, 1H), 5.18 (s, 2H), 3.62 (s, 3H), 2.15 (s, 3H). |
| 311 | 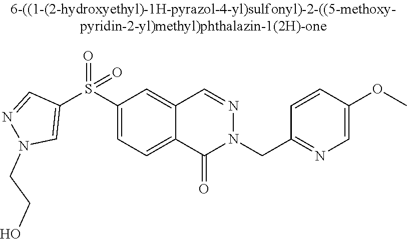 [SMILES: COc1ccc(Cn2ncc3cc(S(=O)(=O)c4cnn(CCO)c4)ccc3c2=O)nc1] | MS (ES+): 442 (M + H)+ 1H NMR (DMSO) δ: 8.62 (d, J = 1.8 Hz, 1H), 8.61 (d, J = 0.6 Hz, 1H), 8.53 (d, J = 0.8 Hz, 1H), 8.43 (d, J = 8.4 Hz, 1H), 8.31 (dd, J = 8.5, 1.9 Hz, 1H), 8.16 (d, J = 2.9 Hz, 1H), 8.05 (d, J = 0.7 Hz, 1H), 7.33 (dd, J = 8.6, 3.0 Hz, 1H), 7.24 (d, J = 8.7 Hz, 1H), 5.38 (s, 2H), 4.94 (t, J = 5.3 Hz, 1H), 4.18 (t, J = 5.3 Hz, 2H), 3.78 (s, 3H), 3.72 (td, J = 5.4, 5.3 Hz, 2H). |
| 312 | 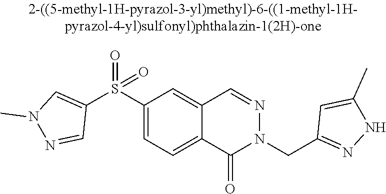 | MS (ES+): 385 (M + H)+ 1H NMR (400 MHz, DMSO) δ 12.30 (s, 1H), 8.59 (d, J = 1.5 Hz, 2H), 8.55 (s, 1H), 8.43 (d, J = 8.4 Hz, 1H), 8.28 (dd, J = 8.4, 1.9 Hz, 1H), 8.01 (d, J = 0.8 Hz, 1H), 5.85 (s, 1H), 5.23 (s, 2H), 3.87 (s, 3H), 2.13 (s, 3H). |
| 313 | 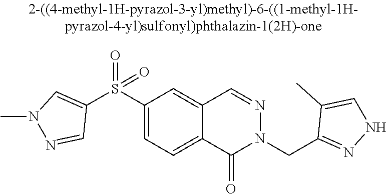 [SMILES: Cc1c[nH]nc1Cn1ncc2cc(S(=O)(=O)c3cnn(C)c3)ccc2c1=O] | MS (ES+): 385 (M + H)+ 1H NMR (400 MHz, DMSO) δ 12.36 (s, 1H), 8.60-8.52 (m, 3H), 8.44 (d, J = 8.4 Hz, 1H), 8.28 (dd, J = 8.4, 1.9 Hz, 1H), 8.01 (s, 1H), 7.41 (s, 1H), 5.28 (s, 2H), 3.87 (s, 3H), 1.95 (s, 3H). |
| 314 | 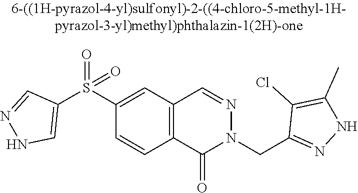 [SMILES: Cc1[nH]nc(Cn2ncc3cc(S(=O)(=O)c4cn[nH]c4)ccc3c2=O)c1Cl] | MS (ES+): 405 (M + H)+ ¹H NMR (400 MHz, DMSO- d₆) δ: 13.91 (s, 1H), 12.79 (s, 1H), 8.60 (d, J = 1.2 Hz, 1H), 8.59 (d, J = 0.4 Hz, 1H), 8.44 (d, J = 8.4 Hz, 1H), 8.34 (s, 2H), 8.32 (dd, J = 8.4 Hz, 1.6 Hz, 1H), 5.30 (s, 2H), 2.14 (s, 3H). |
| 315 | 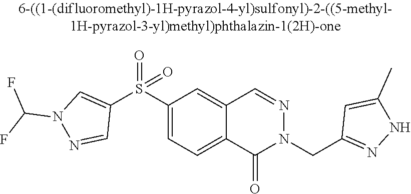 | MS (ES+): 421 (M + H)+ 1H NMR (DMSO) δ: 12.30 (s, 1H), 9.15 (s, 1H), 8.67 (d, J = 1.9 Hz, 1H), 8.60 (s, 1H), 8.46 (d, J = 8.5 Hz, 1H), 8.39 (s, 1H), 8.36 (dd, J = 8.5, 1.9 Hz, 1H), 7.85 (t, J = 58.3 Hz, 1H), 5.85 (s, 1H), 5.23 (s, 2H), 2.12 (s, 3H). |
| 316 | 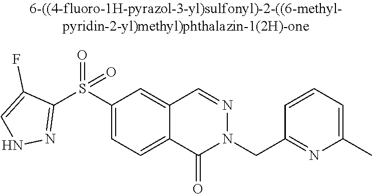 [SMILES: Cc1cccc(Cn2ncc3cc(S(=O)(=O)c4n[nH]cc4F)ccc3c2=O)n1] | MS (ES+): 400 (M + H)+ 1H NMR (400 MHz, DMSO) δ 14.04 (s, 1H), 8.72 (d, J = 0.6 Hz, 1H), 8.68 (d, J = 1.8 Hz, 1H), 8.50 (d, J = 8.4 Hz, 1H), 8.30 (dd, J = 8.4, 1.9 Hz, 1H), 8.17 (d, J = 4.5 Hz, 1H), 7.59 (t, J = 7.7 Hz, 1H), 7.13 (d, J = 7.6 Hz, 1H), 6.96 (d, J = 7.7 Hz, 1H), 5.39 (s, 2H), 2.41 (s, 3H). |
| 317 | 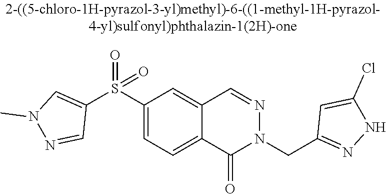 | MS (ES+): 405/407 (M + H)+ 1H NMR (400 MHz, DMSO) δ 13.10 (s, 1H), 8.63 (s, 1H), 8.61 (d, J = 1.9 Hz, 1H), 8.55 (s, 1H), 8.44 (d, J = 8.4 Hz, 1H), 8.30 (dd, J = 8.4, 1.9 Hz, 1H), 8.01 (d, J = 0.8 Hz, 1H), 6.22 (d, J = 1.9 Hz, 1H), 5.32 (s, 2H), 3.87 (s, 3H). |
| 318 | 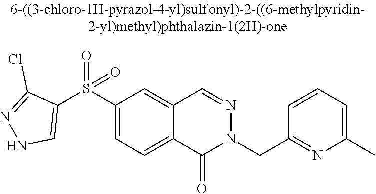 [SMILES: Cc1cccc(Cn2ncc3cc(S(=O)(=O)c4c[nH]nc4Cl)ccc3c2=O)n1] | MS (ES+): 416/418 (M + H)+ 1H NMR (400 MHz, DMSO) δ 14.19 (s, 1H), 8.72 (s, 1H), 8.69 (s, 1H), 8.65 (d, J = 1.8 Hz, 1H), 8.48 (d, J = 8.4 Hz, 1H), 8.30 (dd, J = 8.4, 1.9 Hz, 1H), 7.59 (t, J = 7.7 Hz, 1H), 7.13 (d, J = 7.7 Hz, 1H), 6.96 (d, J = 7.7 Hz, 1H), 5.39 (s, 2H), 2.41 (s, 3H). |
| 319 | 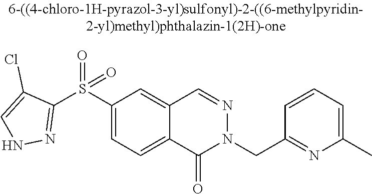 [SMILES: Cc1cccc(Cn2ncc3cc(S(=O)(=O)c4n[nH]cc4Cl)ccc3c2=O)n1] | MS (ES+): 416/418 (M + H)+ 1H NMR (400 MHz, DMSO) δ 8.71 (s, 1H), 8.65 (d, J = 1.8 Hz, 1H), 8.48 (d, J = 8.4 Hz, 1H), 8.28 (dd, J = 8.5, 1.8 Hz, 1H), 8.10 (s, 1H), 7.59 (t, J = 7.7 Hz, 1H), 7.13 (d, J = 7.6 Hz, 1H), 6.95 (d, J = 7.7 Hz, 1H), 5.39 (s, 2H), 2.41 (s, 3H). |
| 320 | 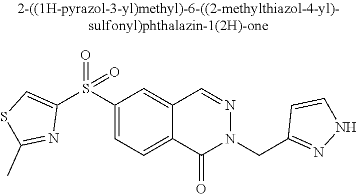 [SMILES: Cc1nc(S(=O)(=O)c2ccc3c(=O)n(Cc4cc[nH]n4)ncc3c2)cs1] | MS (ES+): 388 (M + H)+ 1H NMR (400 MHz, DMSO) δ 12.65 (s, 1H), 8.71-8.63 (m, 3H), 8.48 (d, J = 8.4 Hz, 1H), 8.30 (dd, J = 8.4, 1.9 Hz, 1H), 7.62 (s, 1H), 6.14 (s, 1H), 5.31 (s, 2H), 2.64 (s, 3H). |
| 321 | 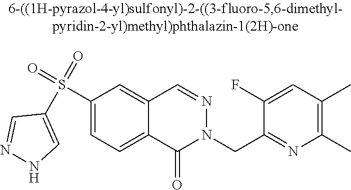 [SMILES: Cc1cc(F)c(Cn2ncc3cc(S(=O)(=O)c4cn[nH]c4)ccc3c2=O)nc1C] | MS (ES+): 414.2 (M + H)+ 1H NMR (400 MHz, DMSO- d6) δ: 13.92 (s, 1H), 8.60 (d, J = 1.6 Hz, 1H), 8.58 (s, 1H), 8.43-8.30 (m, 4H), 7.48 (d, J = 10.4 Hz, 1H), 5.43 (s, 2H), 2.24 (s, 3H), 2.22 (s, 3H). 1H NMR (400 MHz, DMSO- d6 and TFA-d1) δ: 8.61 (d, J = 1.2 Hz, 1H), 8.58 (d, J = 0.4 Hz, 1H), 8.42 (d, J = 8.4 Hz, 1H), 8.35 (br, 2H), 8.31 |
| | | (dd, J = 8.4, 2.0 Hz, 1H), 7.54 |
| | | (dd, J = 10.8 Hz, 1H), 5.45 (s, |
| | | 2H), 2.27 (s, 3H), 2.23 (s, |
| | | 3H). |
| 322 | 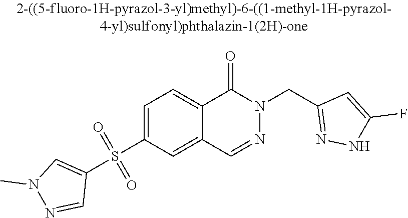 [SMILES: Cn1cc(S(=O)(=O)c2ccc3c(=O)n(Cc4cc(F)[nH]n4)ncc3c2)cn1] | MS (ES+): 389.0 (M + H)+ ¹H NMR (400 MHz, DMSO- d6) δ: 12.49 (br, 1H), 8.63 (s, 1H), 8.61 (d, J = 0.8 Hz, 1H), 8.56 (s, 1H), 8.44 (d, J = 8.4 Hz, 1H), 8.31 (dd, J = 8.4 Hz, 1.6 Hz, 1H), 8.02 (d, J = 0.8 Hz, 1H), 5.87 (dd, J = 6.4 Hz, 2.4 Hz, 1H), 2.28 (d, J = 6.8 Hz, 2H), 3.87 (s, 3H). |
| 323 | 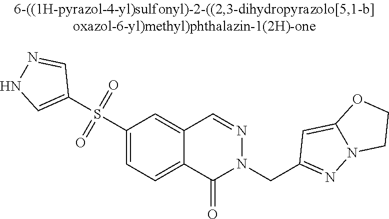 [SMILES: O=c1c2ccc(S(=O)(=O)c3cn[nH]c3)cc2cnn1Cc1cc2n(n1)CCO2] | MS (ES+): 399.2 (M + H)+ 1H NMR (400 MHz, DMSO- d6) δ: 13.91 (s, 1H), 8.60 (d, J = 1.6 Hz, 1H), 8.59 (d, J = 0.4 Hz, 1H), 8.44-8.29 (m, 4H), 5.31 (s, 1H), 5.16 (s, 2H), 4.99 (t, J = 8.0 Hz, 2H), 4.17 (t, J = 8.0 Hz, 2H). 1H NMR (400 MHz, DMSO- d6 + TFA-D) δ: 8.60 (d, J = 0.4 Hz, 1H), 8.59 (d, J = 2.0 Hz, 1H), 8.43 (d, J = 8.4 Hz, 1H), 8.34 (s, 2H), 8.30 (dd, J = 8.4, 2.0 Hz, 1H), 5.31 (s, |
| | | 1H), 5.16 (s, 2H), 4.99 (t, |
| | | J = 7.6 Hz, 2H), 4.17 (d, J = |
| | | 8.4 Hz, 2H). |
| 324 | 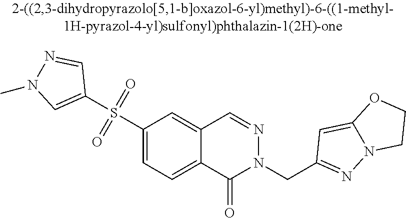 [SMILES: Cn1cc(S(=O)(=O)c2ccc3c(=O)n(Cc4cc5n(n4)CCO5)ncc3c2)cn1] | MS (ES+): 413.2 (M + H)+ 1H NMR (400 MHz, DMSO- d6) δ: 8.59 (s, 2H), 8.56 (s, 1H), 8.43 (d, J = 8.4 Hz, 1H), 8.29 (dd, J = 8.4, 2.0 Hz, 1H), 8.02 (d, J = 0.8 Hz, 1H), 5.31 (s, 1H), 5.16 (s, 2H), 4.99 (t, J = 7.8 Hz, 2H), 4.17 (t, J = 8.0 Hz, 2H), 3.87 (s, 3H). |
| 325 | 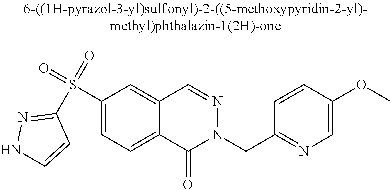 [SMILES: COc1ccc(Cn2ncc3cc(S(=O)(=O)c4cc[nH]n4)ccc3c2=O)nc1] | MS (ES+): 398.2 (M + H)+ 1H NMR (400 MHz, DMSO- d6) δ: 13.95 (s, 1H), 8.66 (s, 1H), 8.65 (d, J = 1.6 Hz, 1H), 8.45 (d, J = 8.4 Hz, 1H), 8.29 (dd, J = 8.4, 2.0 Hz, 1H), 8.17 (d, J = 2.8 Hz, 1H), 8.03 (d, J = 2.4 Hz, 1H), 7.33 (dd, J = 8.8, 3.2 Hz, 1H), 7.24 (d, J = 8.4 Hz, 1H), 7.69 (d, J = 2.4 Hz, 1H), 5.39 (s, 2H), 3.78 (s, 3H). |
| 326 | 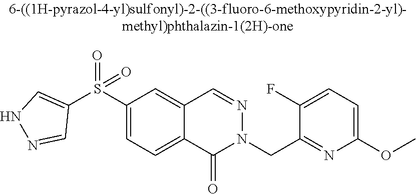 [SMILES: COc1ccc(F)c(Cn2ncc3cc(S(=O)(=O)c4cn[nH]c4)ccc3c2=O)n1] | MS (ES+): 416.0 (M + H)+ 1H NMR (400 MHz, DMSO- d₆ + TFA-d) δ: 8.64 (d, J = 1.6 Hz, 1H), 8.63 (s, 1H), 8.46 (d, J = 8.4 Hz, 1H), 8.36 (s, 2H), 8.34 (dd, J = 8.4, 2.0 Hz, 1H), 7.66 (t, J = 8.8 Hz, 1H), 6.77 (dd, J = 8.8, 2.8 Hz, 1H), 5.46 (d, J = 1.2 Hz, 1H), 3.50 (s, 3H). |
| 327 | 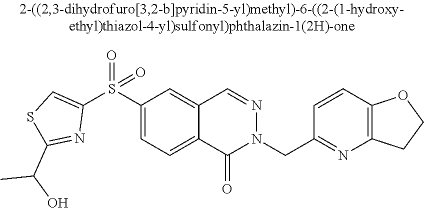 [SMILES: CC(O)c1nc(S(=O)(=O)c2ccc3c(=O)n(Cc4ccc5c(n4)CCO5)ncc3c2)cs1] | MS (ES+): 471 (M + H)+ 1H NMR (400 MHz, DMSO) δ 8.76 (s, 1H), 8.68 (d, J = 1.9 Hz, 1H), 8.67 (s, 1H), 8.47 (d, J = 8.4 Hz, 1H), 8.31 (dd, J = 8.5, 1.9 Hz, 1H), 7.06 (d, J = 8.3 Hz, 1H), 6.99 (d, J = 8.3 Hz, 1H), 6.38 (d, J = 5.1 Hz, 1H), 5.34 (s, 2H), 4.94-4.84 (m, 1H), 4.59 (t, J = 8.9 Hz, 2H), 3.18 (t, J = 8.8 Hz, 2H), 1.37 (d, J = 6.5 Hz, 3H). |
| 328 | 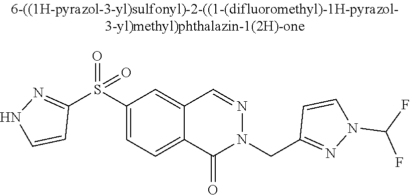 [SMILES: O=c1c2ccc(S(=O)(=O)c3cc[nH]n3)cc2cnn1Cc1ccn(C(F)F)n1] | MS (ES+): 407.0 (M + H)+ 1H NMR (400 MHz, DMSO- d6) δ: 13.95 (s, 1H), 8.67 (s, 1H), 8.65 (d, J = 1.6 Hz, 1H), 8.46 (d, J = 8.4 Hz, 1H), 8.30 (dd, J = 8.4, 1.6 Hz, 1H), 8.15 (d, J = 2.8 Hz, 1H), 8.03 (d, J = 2.0 Hz, 1H), 7.74 (t, J = 59.2 Hz, 1H), 6.96 (d, J = 2.4 Hz, 1H), 6.43 (d, J = 2.8 Hz, 1H), 5.35 (s, 2H). |
| 329 | 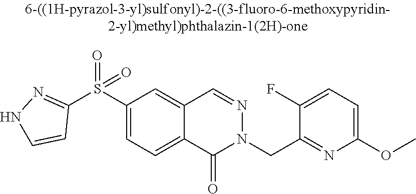 [SMILES: COc1ccc(F)c(Cn2ncc3cc(S(=O)(=O)c4cc[nH]n4)ccc3c2=O)n1] | MS (ES+): 416.2 (M + H)+ 1H NMR (400 MHz, DMSO- d6) δ: 14.00 (s, 1H), 8.68 (d, J = 0.4 Hz, 1H), 8.65 (d, J = 1.6 Hz, 1H), 8.45 (d, J = 9.2 Hz, 1H), 8.30 (dd, J = 8.4, 1.6 Hz, 1H), 8.04 (d, J = 2.4 Hz, 1H), 7.68 (t, J = 8.8 Hz, 1H), 6.98 (d, J = 2.4 Hz, 1H), 6.76 (dd, J = 8.8, 2.8 Hz, 1H), 5.44 (d, J = 1.2 Hz, 2H), 3.48 (s, 3H). |
| 330 | 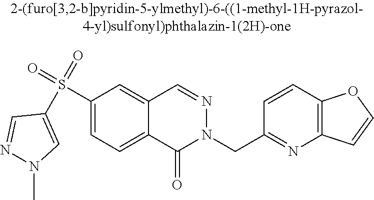 [SMILES: Cn1cc(S(=O)(=O)c2ccc3c(=O)n(Cc4ccc5occc5n4)ncc3c2)cn1] | MS (ES+): 422.2 (M + H)+ ¹H NMR (400 MHz, DMSO- d6) δ: 8.65-8.62 (m, 2H), 8.57 (s, 1H), 8.45 (d, J = 8.4 Hz, 1H), 8.32-8.28 (m, 2H), 8.03 (s, 1H), 7.98 (d, J = 8.4 Hz, 1H), 7.26 (d, J = 8.8 Hz, 1H), 7.05 (d, J = 6.0 Hz, 1H), 5.55 (s, 2H), 3.88 (s, 3H). |
| 331 | 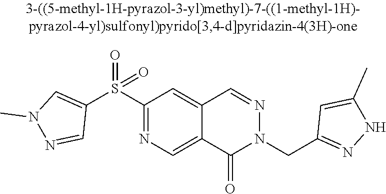 | MS (ES+): 386.2 (M + H)+ ¹H NMR (400 MHz, DMSO- d6) δ: 9.51 (s, 1H), 8.69 (d, J = 0.4 Hz, 1H), 8.66 (d, J = 0.8 Hz, 1H), 8.58 (s, 1H), 7.96 (d, J = 0.4 Hz, 1H), 5.89 (s, 1H), 5.25 (s, 2H), 2.14 (s, 3H). |
| 332 | 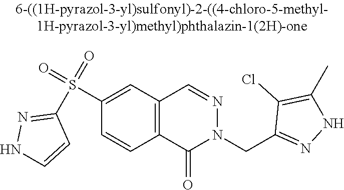 [SMILES: Cc1[nH]nc(Cn2ncc3cc(S(=O)(=O)c4cc[nH]n4)ccc3c2=O)c1Cl] | MS (ES+): 405.2 (M + H)+ ¹H NMR (400 MHz, DMSO- d6) δ: 13.98 (br, 1H), 12.79 (br, 1H), 8.62 (s, 2H), 8.46 (d, J = 8.4 Hz, 1H), 8.29 (dd, J = 8.4 Hz, 1.6 Hz, 1H), 8.03 (d, J = 2.4 Hz, 1H), 6.96 (d, J = 2.4 Hz, 1H), 5.28 (s, 2H), 2.14 (s, 3H). |
| 333 | 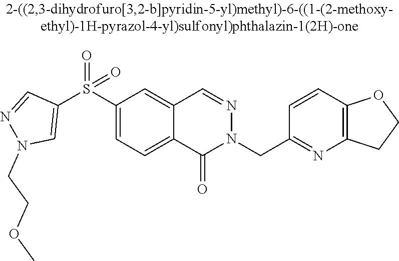 [SMILES: COCCn1cc(S(=O)(=O)c2ccc3c(=O)n(Cc4ccc5c(n4)CCO5)ncc3c2)cn1] | MS (ES+): 468 (M + H)+ 1H NMR (DMSO) δ: 8.63 (d, J = 1.8 Hz, 1H), 8.62 (s, 1H), 8.57 (s, 1H), 8.44 (d, J = 8.4 Hz, 1H), 8.31 (dd, J = 8.4, 1.8 Hz, 1H), 8.06 (d, J = 0.7 Hz, 1H), 7.05 (d, J = 8.2 Hz, 1H), 6.98 (d, J = 8.3 Hz, 1H), 5.34 (s, 2H), 4.59 (t, J = 8.9 Hz, 2H), 4.31 (t, J = 5.1, 2H), 3.71-3.64 (m, 2H), 3.19 (s, 3H), 3.17 (t, J = 8.9, 2H). |
| 334 | 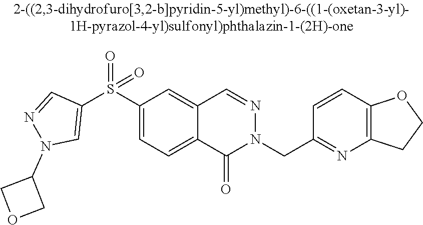 [SMILES: O=c1c2ccc(S(=O)(=O)c3cnn(C4COC4)c3)cc2cnn1Cc1ccc2c(n1)CCO2] | MS (ES+): 466 (M + H)+ 1H NMR (DMSO) δ: 8.73 (s, 1H), 8.63 (d, J = 1.8 Hz, 1H), 8.62 (s, 1H), 8.44 (d, J = 8.4 Hz, 1H), 8.32 (dd, J = 8.4, 1.8 Hz, 1H), 8.20 (s, 1H), 7.06 (d, J = 8.3 Hz, 1H), 6.98 (d, J = 8.3 Hz, 1H), 5.67-5.56 (m, 1H), 5.34 (s, 2H), 4.88 (t, J = 7.2, 7.2 Hz, 2H), 4.83 (t, J = 6.6, 6.6 Hz, 2H), 4.59 (t, J = 8.9 Hz, 2H), 3.17 (t, J = 8.8 Hz, 2H). |
| 335 | 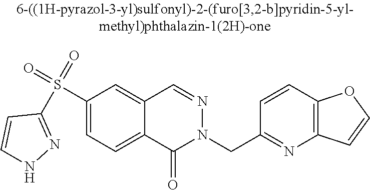 [SMILES: O=c1c2ccc(S(=O)(=O)c3cc[nH]n3)cc2cnn1Cc1ccc2occc2n1] | MS (ES+): 408.3 (M + H)+ 1H NMR (400 MHz, DMSO- d6) δ: 14.00 (s, 1H), 8.69 (s, 1H), 8.67 (d, J = 1.6 Hz, 1H), 8.47 (d, J = 8.4 Hz, 1H), 8.30 (dd, J = 8.4, 1.6 Hz, 1H), 8.29 (d, J = 2.4 Hz, 1H), 8.04 (d, J = 2.4 Hz, 1H), 7.98 (dd, J = 8.8, 1.2 Hz, 1H), 7.27 (d, J = 8.8 Hz, 1H), 7.06 (dd, J = 2.4, 1.2 Hz, 1H), 6.98 (d, J = 2.4 Hz, 1H), 5.55 (s, 2H). |
| 336 | 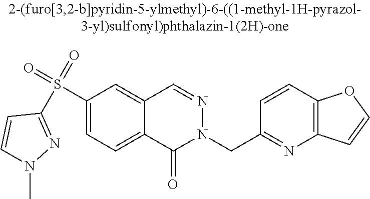 [SMILES: Cn1ccc(S(=O)(=O)c2ccc3c(=O)n(Cc4ccc5occc5n4)ncc3c2)n1] | MS (ES+): 422.2 (M + H)+ ¹H NMR (400 MHz, DMSO- d₆) δ: 8.69 (s, 1H), 8.66 (d, J = 1.6 Hz, 1H), 8.47 (d, J = 8.4 Hz, 1H), 8.31-8.28(m, 2H), 8.00-7.97(m, 2H), 7.27 (d, J = 8.4 Hz, 1H), 7.05(dd, J₁ = 0.8 Hz, J₁ = 2.0 Hz, 1H), 6.97(d, J = 2.4 Hz, 1H), 5.55 (s, 2H), 3.91(s, 3H). |
| 337 | 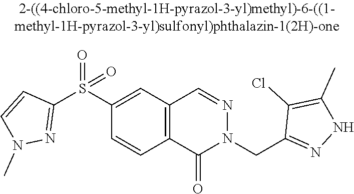 [SMILES: Cc1[nH]nc(Cn2ncc3cc(S(=O)(=O)c4ccn(C)n4)ccc3c2=O)c1Cl] | MS (ES+): 419.2 (M + H)+ ¹H NMR (400 MHz, DMSO- d6) δ: 12.78 (br, 1H), 8.62 (s, 2H), 8.47 (d, J = 8.4 Hz, 1H), 8.28 (dd, J = 8.4 Hz, 2.0 Hz, 1H), 7.96 (d, J = 2.4 Hz, 1H), 6.96 (d, J = 2.4 Hz, 1H), 5.29 (s, 2H), 3.91 (s, 3H), 2.14 (s, 3H). |
| 338 | 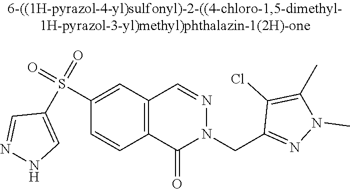 [SMILES: Cc1c(Cl)c(Cn2ncc3cc(S(=O)(=O)c4cn[nH]c4)ccc3c2=O)nn1C] | MS (ES+): 419.0 (M + H)+ ¹H NMR (400 MHz, DMSO- d₆ + CF₃COOD) δ: 13.92 (br, 1H), 8.60 (d, J = 2.0 Hz, 1H), 8.58 (s, 1H), 8.45 (d, J = 8.4 Hz, 1H), 8.34 (s, 2H), 8.32 (dd, J = 8.4 Hz, 2.0 Hz, 1H), 5.28 (s, 2H), 3.68 (s, 3H), 2.19 (s, 3H). |
| 339 | 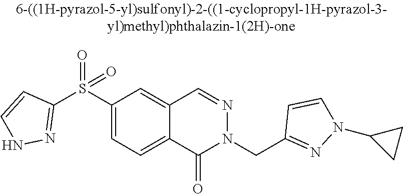 [SMILES: O=c1c2ccc(S(=O)(=O)c3cc[nH]n3)cc2cnn1Cc1ccn(C2CC2)n1] | MS (ES+): 397 (M + H)+ 1H NMR (400 MHz, DMSO) δ 13.98 (s, 1H), 8.64 (d, J = 0.7 Hz, 1H), 8.63 (d, J = 1.9 Hz, 1H), 8.45 (d, J = 8.4 Hz, 1H), 8.28 (dd, J = 8.4, 1.8 Hz, 1H), 8.02 (d, J = 2.4 Hz, 1H), 7.64 (d, J = 2.3 Hz, 1H), 6.95 (d, J = 2.4 Hz, 1H), 6.08 (d, J = 2.3 Hz, 1H), 5.24 (s, 2H), 3.67-3.57 (m, 1H), 0.99-0.94 (m, 2H), 0.93-0.86 (m, 2H). |
| 340 | 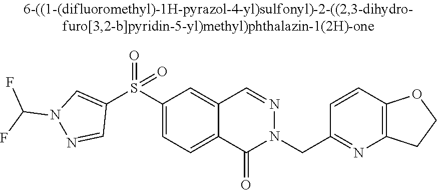 [SMILES: O=c1c2ccc(S(=O)(=O)c3cnn(C(F)F)c3)cc2cnn1Cc1ccc2c(n1)CCO2] | MS (ES+): 460 (M + H)+ 1H NMR (400 MHz, DMSO) δ 9.16 (d, J = 0.7 Hz, 1H), 8.68 (d, J = 1.8 Hz, 1H), 8.62 (d, J = 0.7 Hz, 1H), 8.48-8.44 (m, 1H), 8.40 (s, 1H), 8.37 (dd, J = 8.4, 1.9 Hz, 1H), 7.85 (t, J = 58.3 Hz, 1H), 7.06 (d, J = 8.3 Hz, 1H), 6.98 (d, J = 8.3 Hz, 1H), 5.35 (s, 2H), 4.59 (t, J = 8.9 Hz, 2H), 3.17 (t, J = 8.8 Hz, 2H). |
| 341 | 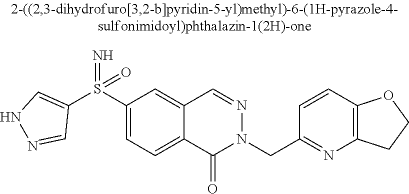 [SMILES: N=S(=O)(c1cn[nH]c1)c1ccc2c(=O)n(Cc3ccc4c(n3)CCO4)ncc2c1] | MS (ES+): 409 (M + H)+ 1H NMR (400 MHz, DMSO) δ 13.62 (s, 1H), 8.60 (s, 1H), 8.59 (d, J = 1.8 Hz, 1H), 8.38 (d, J = 8.4 Hz, 1H), 8.33 (dd, J = 8.4, 1.8 Hz, 1H), 8.12 (s, 2H), 7.05 (d, J = 8.3 Hz, 1H), 6.96 (d, J = 8.3 Hz, 1H), 5.33 (s, 2H), 5.22 (s, 1H), 4.58 (t, J = 8.9 Hz, 2H), 3.17 (t, J = 8.8 Hz, 2H). |
| 342 | 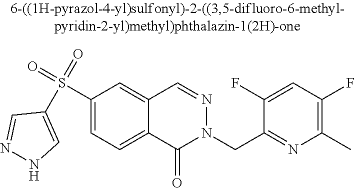 [SMILES: Cc1nc(Cn2ncc3cc(S(=O)(=O)c4cn[nH]c4)ccc3c2=O)c(F)cc1F] | MS (ES+): 418.1 (M + H)+ ¹H NMR (400 MHz, DMSO- d₆ + CF₃CO₂D) δ: 8.63-8.60 (m, 2H), 8.43 (d, J = 8.4 Hz, 1H), 8.35-8.30 (m, 3H), 7.85 (t, J = 8.8 Hz, 1H), 5.47 (s, 2H), 2.29 (s, 3H). |
| 343 | 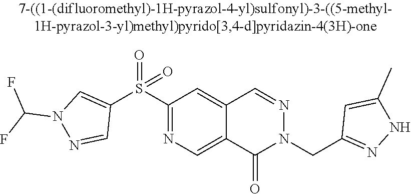 | MS (ES+): 422.2 (M + H)+ ¹H NMR (400 MHz, DMSO- d6) δ: 12.35 (br, 1H), 9.54 (s, 1H), 9.16 (s, 1H), 8.72 (d, J = 8.0 Hz, 2H), 8.35 (s, 1H), 7.88 (t, J = 18.0 Hz, 1H), 5.88 (s, 1H), 5.25 (s, 2H), 2.14 (s, 3H). |
| 344 | 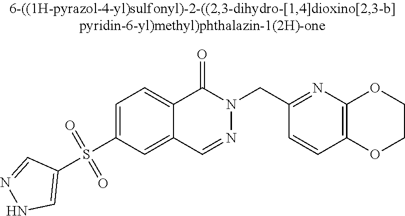 [SMILES: O=c1c2ccc(S(=O)(=O)c3cn[nH]c3)cc2cnn1Cc1ccc2c(n1)OCCO2] | MS (ES+): 426.2 (M + H)+ 1H NMR (400 MHz, DMSO- d6) δ: 13.94 (s, 1H), 8.62 (d, J = 2.0 Hz, 2H), 8.44-8.29 (m, 4H), 7.21 (d, J = 8.0 Hz, 1H), 6.80 (d, J = 8.0 Hz, 1H), 5.26 (s, 2H), 4.35-4.33 (m, 2H), 4.21-4.19 (m, 2H). 1H NMR (400 MHz, DMSO-d6 + TFA-D) δ: 8.62 (d, J = 2.0 Hz, 1H), 8.62 (s, 1H), 8.43 (d, J = 8.4 Hz, 1H), 8.35 (s, 2H), 8.32 (d, J = 8.4, 2.0 Hz, 1H), 7.22 (d, J = 8.0 Hz, 1H), 6.80 (d, |
| | | J = 8.0 Hz, 1H), 5.26 (s, 2H), |
| | | 4.35-4.33 (m, 2H), 4.21-4.19 |
| | | (m, 2H). |
| 345 | 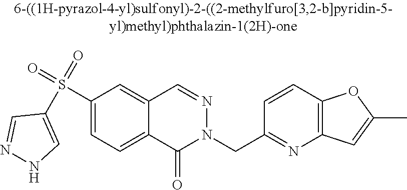 [SMILES: Cc1cc2nc(Cn3ncc4cc(S(=O)(=O)c5cn[nH]c5)ccc4c3=O)ccc2o1] | MS (ES+): 422.2 (M + H)+ ¹H NMR (400 MHz, DMSO- d6 + CF₃COOD) δ 13.92 (br, 1H), 8.68-8.66 (m, 2H), 8.46 (d, J = 8.4 Hz, 1H), 8.37-8.34 (m, 4H), 7.54-7.52 (s, 1H), 6.99 (s, 1H), 5.68 (s, 2H), 2.60 (s, 3H). |
| 346 | 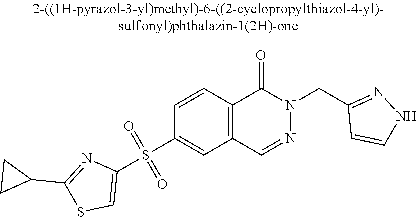 [SMILES: O=c1c2ccc(S(=O)(=O)c3csc(C4CC4)n3)cc2cnn1Cc1cc[nH]n1] | MS (ES+): 414.1 (M + H)+ ¹H NMR (400 MHz, DMSO- d₆ +HCl) δ: 8.70 (s, 1H), 8.67 (d, J = 1.6 Hz, 1H), 8.61 (s, 1H), 8.49 (d, J = 8.4 Hz, 1H), 8.32 (dd, J = 8.4 Hz, 2.0 Hz, 1H), 7.93 (d, J = 2.4 Hz, 1H), 6.42 (d, J = 2.4 Hz, 1H), 5.42 (s, 2H), 2.47-2.44 (m, 1H), 1.17-1.13 (m, 2H), 0.94-0.92 (m, 2H). |
| 347 | 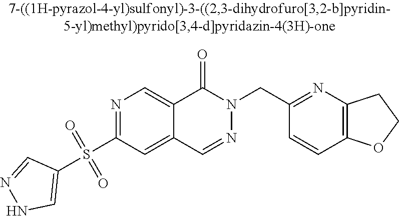 [SMILES: O=c1c2cnc(S(=O)(=O)c3cn[nH]c3)cc2cnn1Cc1ccc2c(n1)CCO2] | MS (ES+): 411.2 (M + H)+ 1H NMR (400 MHz, DMSO- d6) δ: 13.94 (s, 1H), 9.51 (s, 1H), 8.72 (d, J = 0.4 Hz, 1H), 8.68 (d, J = 0.8 Hz, 1H), 8.62 (s, 1H), 8.01 (s, 1H), 7.07 (d, J = 8.4 Hz, 1H), 7.03 (d, J = 8.4 Hz, 1H), 5.36 (s, 2H), 4.59 (t, J = 8.8 Hz, 2H), 3.18 (d, J = 8.8 Hz, 1H). 1H NMR (400 MHz, DMSO-d6 + TFA-D) δ: 9.52 (s, 1H), 8.73 (s, 1H), 8.69 (d, J = 0.8 Hz, 1H), 8.32 (s, 2H), 7.19 (d, J = |
| | | 8.4 Hz, 1H), 7.14 (d, J = 8.4 |
| | | Hz, 1H), 5.40 (s, 2H), 4.64 (t, |
| | | J = 8.8 Hz, 2H), 3.25 (d, J = |
| | | 8.8 Hz, 2H). |
| 348 | 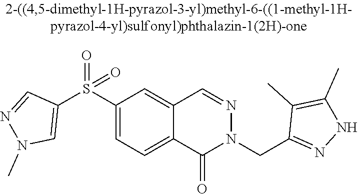 [SMILES: Cc1[nH]nc(Cn2ncc3cc(S(=O)(=O)c4cnn(C)c4)ccc3c2=O)c1C] | MS (ES+): 399.3 (M + H)+ ¹H NMR (400 MHz, DMSO- d6 + CF₃COOD) δ: 8.62 (s, 1H), 8.59 (d, J = 1.6 Hz, 1H), 8.54 (s, 1H), 8.39 (d, J = 8.4 Hz, 1H), 8.27 (dd, J = 8.4 Hz, 2.0 Hz, 1H), 7.97 (s, 1H), 5.24 (s, 2H), 3.83 (s, 3H), 2.24 (s, 3H), 1.92 (s, 3H). |
| 349 | 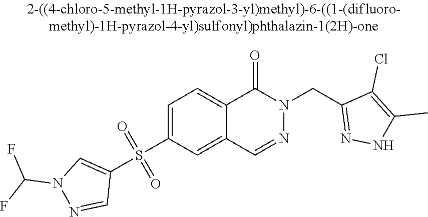 [SMILES: Cc1[nH]nc(Cn2ncc3cc(S(=O)(=O)c4cnn(C(F)F)c4)ccc3c2=O)c1Cl] | MS (ES+): 455.0 (M + H)+ ¹H NMR (400 MHz, DMSO- d6) δ: 12.75 (br, 1H), 9.15 (s, 1H), 8.67 (d, J = 1.6 Hz, 1H), 8.58 (s, 1H), 8.48 (d, J = 8.4 Hz, 1H), 8.40 (s, 1H), 8.38 (d, J = 8.4 Hz, 2.0 H, 1H), 7.84 (t, J = 18.4 Hz, 1H), 5.30 (s, 2H), 2.14 (s, 3H). |
| 350 | 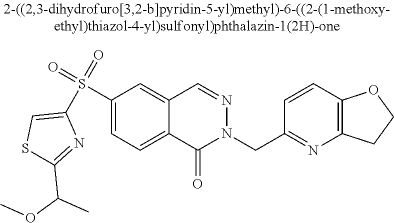 [SMILES: COC(C)c1nc(S(=O)(=O)c2ccc3c(=O)n(Cc4ccc5c(n4)CCO5)ncc3c2)cs1] | MS (ES+): 485.2 (M + H)+ ¹H NMR (400 MHz, DMSO- d₆) δ: 8.85(s, 1H), 8.69(d, J = 1.6 Hz, 1H), 8.67(s, 1H), 8.48(d, J = 8.4 Hz, 1H), 8.33(d, J = 8.4 Hz, 1H), 7.06(d, J = 8.4 Hz, 1H), 7.00(d, J = 8.4 Hz, 1H), 5.34(s, 2H), 4.67(q, J = 6.4 Hz, 1H), 4.59(t, J = 8.8 Hz, 2H), 3.32(s, 3H), 3.18(t, J = 8.8 Hz, 2H), 1.40(d, J = 7.2 Hz, 3H). |
| 351 | 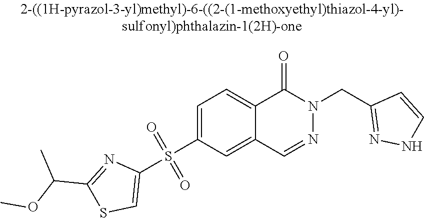 [SMILES: COC(C)c1nc(S(=O)(=O)c2ccc3c(=O)n(Cc4cc[nH]n4)ncc3c2)cs1] | MS (ES+): 432.2 (M + H)+ ¹H NMR (400 MHz, DMSO- d₆ + HCl ) δ: 8.85 (s, 1H), 8.70 (d, J = 1.6 Hz, 2H), 8.49 (d, J = 8.4 Hz, 1H), 8.33 (dd, J = 8.4, 1.6 Hz, 1H), 7.87 (d, J = 2.4 Hz, 1H), 6.37 (d, J = 2.4 Hz, 1H), 5.41 (s, 2H), 4.66 (q, J = 6.4 Hz, 1H), 3.32 (s, 3H), 1.39 (d, J = 6.4 Hz, 3H). |
| 352 | 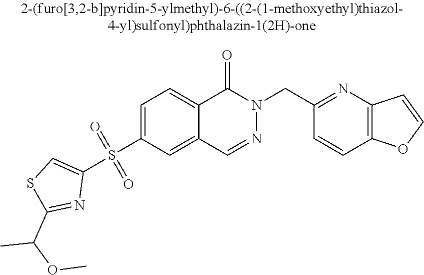 [SMILES: COC(C)c1nc(S(=O)(=O)c2ccc3c(=O)n(Cc4ccc5occc5n4)ncc3c2)cs1] | MS (ES+): 483.2 (M + H)+ ¹H NMR (400 MHz, DMSO- d₆) δ: 8.85 (s, 1H), 8.72- 8.70(m, 2H), 8.49(d, J = 8.4 Hz, 1H), 8.33(dd, J₁ = 1.2 Hz, J₁ = 2.4 Hz, 1H), 8.29(d, J = 2.4 Hz, 1H), 7.99(d, J = 8.4 Hz, 1H), 7.28(d, J = 8.8 Hz, 1H), 7.05(dd, J₁ = 2.0 Hz, J₁ = 8.4 Hz, 1H), 5.55(s, 2H), 4.67(q, J = 6.4 Hz, 1H), 3.32(s, 3H), 1.40(d, J = 6.4 Hz, 3H). |
| 353 | 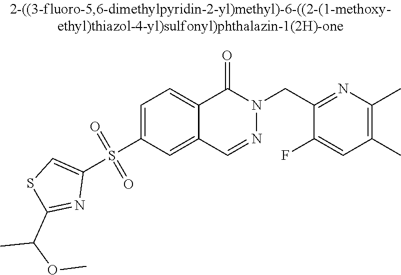 [SMILES: COC(C)c1nc(S(=O)(=O)c2ccc3c(=O)n(Cc4nc(C)c(C)cc4F)ncc3c2)cs1] | MS (ES+): 489.2 (M + H)+ ¹H NMR (400 MHz, DMSO- d₆) δ: 8.85 (s, 1H), 8.68(d, J = 8.4 Hz, 1H), 8.64 (s, 1H), 8.47 (d, J = 8.4 Hz, 1H), 8.33(dd, J = 8.8 Hz, 2.0 Hz, 1H), 7.47 (d, J = 11.2 Hz, 1H), 5.43 (s, 2H), 4.67(q, J = 6.4 Hz, 1H), 3.31(s, 3H), 2.25(s, 3H), 2.22 (s, 3H), 1.40 (d, J = 6.4 Hz, 3H). |
| 354 | 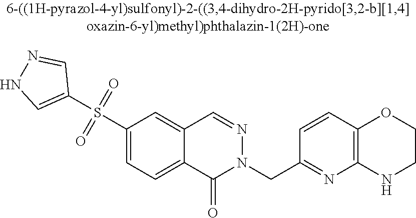 [SMILES: O=c1c2ccc(S(=O)(=O)c3cn[nH]c3)cc2cnn1Cc1ccc2c(n1)NCCO2] | MS (ES+): 425.2 (M + H)+ 1H NMR (400 MHz, DMSO- d₆) δ: 13.93 (br, 1H), 8.61-860 (m, 2H), 8.43 (d, J = 8.4 Hz, 1H), 8.30 (dd, J = 8.4, 2.0 Hz, 2H), 6.83 (d, J = 7.6 Hz, 1H), 6.69 (s, 1H), 6.26 (d, J = 7.6 Hz, 1H), 5.16 (s, 2H), 4.05 (t, J = 4.4 Hz, 2H), 3.28-3.27 (m, 2H). |
| 355 | 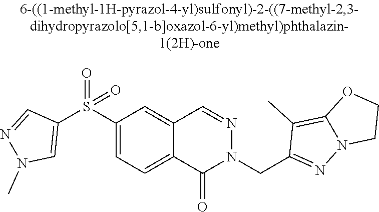 [SMILES: Cc1c(Cn2ncc3cc(S(=O)(=O)c4cnn(C)c4)ccc3c2=O)nn2c1OCC2] | MS (ES+): 427 (M + H)+ 1H NMR (DMSO) δ: 8.60- 8.53 (m, 3H), 8.43 (d, J = 8.4 Hz, 1H), 8.28 (dd, J = 8.4, 1.9 Hz, 1H), 8.01 (d, J = 0.8 Hz, 1H), 5.17 (s, 2H), 4.96 (dd, J = 8.6, 7.2 Hz, 2H), 4.12 (dd, J = 8.5, 7.2 Hz, 2H), 3.87 (s, 3H), 1.78 (s, 3H) |
| 356 | 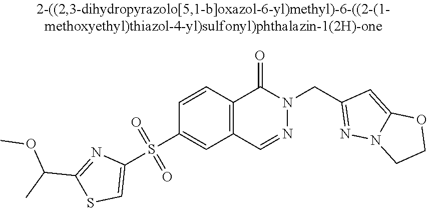 [SMILES: COC(C)c1nc(S(=O)(=O)c2ccc3c(=O)n(Cc4cc5n(n4)CCO5)ncc3c2)cs1] | MS (ES+): 474.2 (M + H)+ 1H NMR (400 MHz, DMSO- d₆) δ: 8.84 (s, 1H), 8.67 (d, J = 1.6 Hz, 1H), 8.65 (br, 1H), 8.48 (d, J = 8.4 Hz, 1H), 8.31 (dd, J = 8.4, 2.0 Hz, 1H), 5.32 (s, 1H), 5.17 (s, 2H), 5.01- 4.97 (m, 2H), 4.66 (d, J = 6.8 Hz, 1H), 4.17 (t, J = 8.0 Hz, 2H), 3.31 (s, 3H), 1.39 (d, J = 6.8 Hz, 3H) |
| 357 | 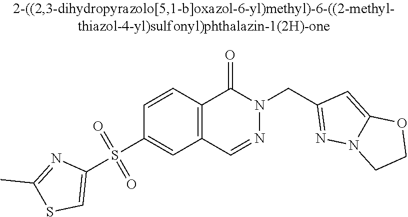 [SMILES: Cc1nc(S(=O)(=O)c2ccc3c(=O)n(Cc4cc5n(n4)CCO5)ncc3c2)cs1] | MS (ES+): 430.2 (M + H)+ 1H NMR (400 MHz, DMSO- d₆) δ: 8.70 (s, 1H), 8.66 (d, J = 1.6 Hz, 1H), 8.65 (s, 1H), 8.47 (d, J = 8.4 Hz, 1H), 8.30 (dd, J = 8.4, 1.6 Hz, 1H), 5.32 (s, 1H), 5.17 (s, 2H), 4.99 (t, J = 8.0 Hz, 2H), 4.17 (t, J = 8.0 Hz, 2H), 2.65 (s, 3H). |
| 358 | 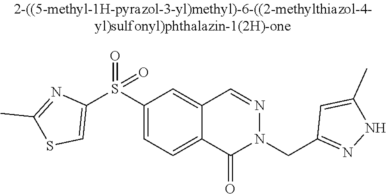 [SMILES: Cc1cc(Cn2ncc3cc(S(=O)(=O)c4csc(C)n4)ccc3c2=O)n[nH]1] | MS (ES+): 402.2 (M + H)+ ¹H NMR (400 MHz, DMSO- d₆) δ: 12.31 (br, 1H), 8.70 (s, 1H), 8.66-8.65 (m, 2H), 8.48 (d, J = 8.4 Hz, 1H), 8.30 (dd, J = 8.4, 2.0 Hz, 1H), 5.86 (s, 1H), 5.23 (s, 2H), 2.64 (s, 3H), 2.14 (br, 3H). |
| 359 | 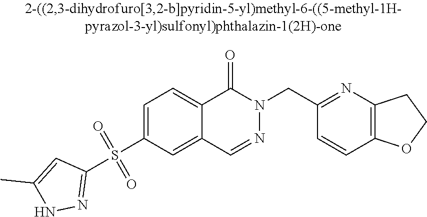 [SMILES: Cc1cc(S(=O)(=O)c2ccc3c(=O)n(Cc4ccc5c(n4)CCO5)ncc3c2)n[nH]1] | MS (ES+): 424.2 (M + H)+ ¹H NMR (400 MHz, DMSO- d₆ + CF₃CO₂D) δ: 8.68 (s, 1H), 8.63 (d, J = 1.2 Hz, 1H), 8.46 (d, J = 8.4 Hz, 1H), 8.27 (dd, J₁ = 2.0 Hz, J₂ = 8.4 Hz, 1H), 7.21-7.10(m, 2H), 6.69 (s, 1H), 5.39 (s, 2H), 4.64 (t, J = 8.8 Hz, 1H), 3.27-3.24 (m, 2H), 2.27 (s, 3H). |
| 360 | 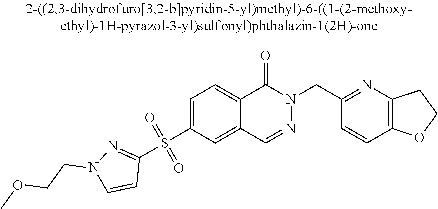 [SMILES: COCCn1ccc(S(=O)(=O)c2ccc3c(=O)n(Cc4ccc5c(n4)CCO5)ncc3c2)n1] | MS (ES+): 468.3 (M + H)+ ¹H NMR (400 MHz, DMSO- d₆) δ: 8.66-8.64 (m, 2H), 8.46 (d, J = 8.4 Hz, 1H), 8.28 (dd, J₁ = 2.0 Hz, J₂ = 8.4 Hz, 1H), 7.99 (d, J = 2.4 Hz, 1H), 7.05 (d, J = 8.0 Hz, 1H), 6.98 (d, J = 8.0 Hz, 1H), 8.95 (d, J = 1.6 Hz, 1H), 5.34 (s, 2H), 4.59 (t, J = 8.8 Hz, 2H), 4.35 (t, J = 5.6 Hz, 2H), 3.66 (t, J = 5.6 Hz, 2H), 3.20-3.15 (m, 5H). |
| 361 | 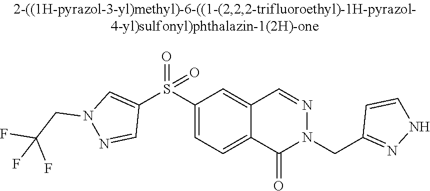 | MS (ES+): 439 (M + H)+ 1H NMR (DMSO) δ: 12.65 (s, 1H), 8.73 (s, 1H), 8.68- 8.59 (m, 2H), 8.46 (d, J = 8.4 Hz, 1H), 8.33 (dd, J = 8.4, 1.9 Hz, 1H), 8.22 (s, 1H), 7.62 (s, 1H), 6.14 (d, J = 2.2 Hz, 1H), 5.32 (s, 2H), 5.24 (q, J = 9.0 Hz, 2H) |
| 362 | 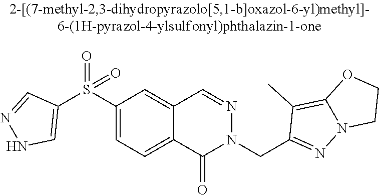 [SMILES: Cc1c(Cn2ncc3cc(S(=O)(=O)c4cn[nH]c4)ccc3c2=O)nn2c1OCC2] | MS (ES+): 413 (M + H)+ 1H NMR (DMSO) δ: 13.90 (s, 1H), 8.60-8.55 (m, 2H), 8.43 (d, J = 8.4 Hz, 1H), 88.26-8.37 (s (br), 2H), 8.29 (dd, J = 8.4, 1.9 Hz, 1H), 5.17 (s, 2H), 4.96 (dd, J = 8.6, 7.2 Hz, 2H), 4.12 (dd, J = 8.6, 7.2 Hz, 2H), 1.78 (s, 3H). |
| 363 | 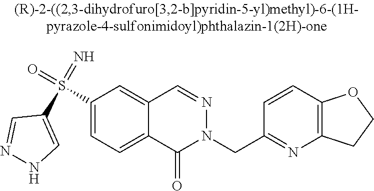 [SMILES: N=[S@](=O)(c1cn[nH]c1)c1ccc2c(=O)n(Cc3ccc4c(n3)CCO4)ncc2c1] | Same as racemic |
| 364 | 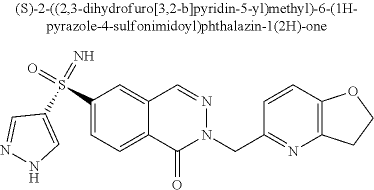 [SMILES: N=[S@@](=O)(c1cn[nH]c1)c1ccc2c(=O)n(Cc3ccc4c(n3)CCO4)ncc2c1] | Same as racemic |

### Biological Example 1—Human PKM2 Activation Assay

### Measuring In Vitro Activation of Recombinant Human PKM2

Compound activation of recombinant human PKM2 pyruvate kinase activity was determined by biochemical assay. N-terminal His-tagged hPKM2 was sourced from R&D Systems and its substrates phosphoenolpyruvate (PEP) and ADP from Sigma-Aldrich and 2Bscientific Ltd, respectively. The Kinase-Glo® Plus luminescence assay was from Promega. All other reagents were from Sigma-Aldrich. Test Compounds were prepared as 10 mM DMSO stocks and dilution series prepared in DMSO for direct dilution into Assay Buffer comprising 50 mM imidazole, 50 mM KCl, 7 mM MgCl₂, 0.01% Tween20, 0.05% BSA (pH 7.2).

### Assay Procedure

Human PKM2 was diluted into Assay Buffer comprising 50 mM imidazole, 50 mM KCl, 7 mM MgCl₂, 0.01% Tween20, 0.05% BSA (pH 7.2) to a final concentration of 5 μM. Enzyme-Assay Buffer mix was dispensed into a 384-well shallow-well white-walled plate (PerkinElmer) and Test Compounds added by acoustic dispense (Echo®, Labcyte Inc.). Following 10 minutes' incubation at room temperature, the enzyme reaction was initiated by acoustic dispensing of ADP+PEP substrate to final concentrations of 254 μM ADP and 53 μM ADP.

After 60 minutes' incubation on an orbital shaker (300 rpm, 26° C.), enzyme activity was quantified by the luminescent detection of generated ATP. Kinase-Glo® Plus reagent was added to each well and the plates incubated for a further 15 minutes on an orbital shaker in the dark (300 rpm, 26° C.) before luminescence measurement on a plate reader (PHERAstar® FSX, BMG Labtech).

Percentage activation was calculated by normalising fluorescence signals to plate LOW (DMSO vehicle) and HIGH (5 μM TEPP-46) controls. EC₅₀ and Eₘₐₓ values were determined from 4-parameter logistic fits of compound concentration-response curves.

A number of Example compounds of formula (Ia) were tested and the results are shown in Table 1 below.

TABLE 1
PKM2 EC₅₀ values (μM) and Eₘₐₓ values (%)

| | hPKM2 | |
|---|---|---|
| | EC₅₀ | hPKM2 Eₘₐₓ |
| Compound | (μM) | (%) |
| Mitapivat | 0.3208 | 55.05 |
| Example 1 | 0.0276 | 128.8 |
| Example 2 | 0.0664 | 97 |
| Example 3 | 0.5659 | 92.62 |
| Example 4 | 0.0539 | 106.1 |
| Example 5 | 0.0607 | 101.5 |
| Example 6 | 0.0389 | 109.2 |
| Example 7 | 0.1772 | 99.83 |
| Example 8 | 0.9315 | 80.84 |
| Example 9 | 0.3623 | 105.1 |
| Example 10 | 0.1658 | 98.75 |
| Example 11 | 0.0724 | 99.01 |
| Example 12 | 0.0636 | 92 |
| Example 13 | 0.1171 | 113.1 |
| Example 14 | 0.1467 | 95.52 |
| Example 15 | 0.2431 | 124.4 |
| Example 16 | 0.1537 | 92.57 |
| Example 17 | 0.1124 | 54.89 |
| Example 18 | 0.8412 | 98.55 |
| Example 19 | 0.1327 | 65.27 |
| Example 20 | 0.2488 | 90.69 |
| Example 21 | 0.1314 | 101.8 |
| Example 22 | 0.1023 | 86.06 |
| Example 23 | 0.2653 | 112.5 |
| Example 24 | 0.1067 | 123.1 |
| Example 25 | 0.1977 | 126.1 |
| Example 26 | 0.8377 | 105.6 |
| Example 27 | 0.07 | 122.1 |
| Example 28 | 0.1617 | 103.1 |
| Example 29 | 0.0337 | 85.03 |
| Example 30 | 0.7392 | 99.44 |
| Example 31 | 0.8092 | 74.88 |
| Example 32 | 0.1629 | 99.57 |
| Example 33 | 0.0633 | 105 |
| Example 34 | 0.5058 | 79.03 |
| Example 35 | 0.1828 | 109.6 |
| Example 36 | 0.2065 | 70.04 |
| Example 37 | 0.1329 | 117.6 |
| Example 38 | 0.0401 | 115.1 |
| Example 39 | 0.1502 | 71.92 |
| Example 40 | 0.0427 | 90 |
| Example 41 | 0.0421 | 111 |
| Example 42 | 0.0645 | 102.9 |
| Example 43 | 0.0425 | 111.4 |
| Example 44 | 0.3665 | 63.02 |
| Example 45 | 0.4347 | 83.5 |
| Example 46 | 0.2335 | 76 |
| Example 47 | 0.0255 | 87.93 |
| Example 48 | 0.0552 | 85.85 |
| Example 49 | 0.1224 | 98.19 |
| Example 50 | 0.0706 | 97.5 |
| Example 51 | 0.0241 | 103.8 |
| Example 52 | 0.0597 | 100.6 |
| Example 53 | 0.0591 | 89.21 |
| Example 54 | 0.0455 | 112.3 |
| Example 55 | 0.5659 | 92.62 |
| Example 56 | 0.0519 | 98.04 |
| Example 57 | 0.055 | 108.6 |
| Example 58 | 0.4769 | 88.62 |
| Example 59 | 0.0689 | 107.1 |
| Example 60 | 0.1 | 110.3 |
| Example 61 | 0.564 | 67.17 |
| Example 62 | 0.0387 | 89.49 |
| Example 63 | 0.1675 | 101.2 |
| Example 64 | 0.045 | 96.42 |
| Example 65 | 0.3598 | 98.57 |
| Example 66 | 0.1152 | 88.45 |
| Example 67 | 0.1847 | 91.43 |
| Example 68 | 0.0708 | 117.2 |
| Example 69 | 1.169 | 75.46 |
| Example 70 | 0.6631 | 96.91 |
| Example 71 | 0.1152 | 102.1 |
| Example 72 | 0.0359 | 102.7 |
| Example 73 | 0.0302 | 99.86 |
| Example 74 | 0.2177 | 91.23 |
| Example 75 | 0.0477 | 107.7 |
| Example 76 | 0.1046 | 100.6 |
| Example 77 | 0.063 | 105.9 |
| Example 78 | 0.8022 | 99 |
| Example 79 | 0.203 | 90.15 |
| Example 80 | 0.7987 | 93.21 |
| Example 81 | 0.0498 | 98.93 |
| Example 82 | 0.0809 | 87.38 |
| Example 83 | 0.4663 | 78.09 |
| Example 84 | 0.1147 | 93.09 |
| Example 85 | 0.0715 | 112.7 |
| Example 86 | 0.7647 | 92.21 |
| Example 87 | 0.4746 | 85.75 |
| Example 88 | 0.6181 | 93.15 |
| Example 89 | 0.0508 | 101.2 |
| Example 90 | 0.0557 | 106 |
| Example 91 | 0.5541 | 89.88 |
| Example 92 | 0.1579 | 98.93 |
| Example 93 | 0.4115 | 108 |
| Example 94 | 0.5878 | 71.31 |
| Example 95 | 0.0954 | 115.5 |
| Example 96 | 0.0626 | 115.7 |
| Example 97 | 0.7152 | 99.13 |
| Example 98 | 0.2568 | 82.04 |
| Example 99 | 0.1384 | 107.2 |
| Example 100 | 0.6766 | 70.82 |
| Example 101 | 0.2401 | 74.53 |
| Example 102 | 0.0699 | 94.87 |
| Example 103 | 0.0413 | 98.77 |
| Example 104 | 0.0565 | 103.5 |
| Example 105 | 0.8134 | 103.3 |
| Example 106 | 0.1418 | 92.96 |
| Example 107 | 0.5851 | 103.4 |
| Example 108 | 0.1781 | 131.6 |
| Example 109 | 0.1413 | 96.51 |
| Example 110 | 0.0686 | 118.8 |
| Example 111 | 0.243 | 124.3 |
| Example 112 | 0.0213 | 66.39 |
| Example 113 | 0.163 | 133 |
| Example 114 | 0.2075 | 97.06 |
| Example 115 | 0.2765 | 69.78 |
| Example 116 | 0.0595 | 96.13 |
| Example 117 | 0.1766 | 77.05 |
| Example 118 | 0.0602 | 93.18 |
| Example 119 | 0.348 | 98.77 |
| Example 120 | 0.2338 | 103.2 |
| Example 121 | 0.6203 | 115.7 |
| Example 122 | 0.5896 | 60 |
| Example 123 | 0.1228 | 74.37 |
| Example 124 | 0.0313 | 106.7 |
| Example 125 | 0.4578 | 88.78 |
| Example 126 | 0.0189 | 95.35 |
| Example 127 | 0.0433 | 68.29 |
| Example 128 | 0.0345 | 80.77 |
| Example 130 | 0.2245 | 57.07 |
| Example 131 | 0.2996 | 99.68 |
| Example 132 | 0.2893 | 95.12 |
| Example 133 | 0.4436 | 77.27 |
| Example 134 | 0.0881 | 102.5 |
| Example 135 | 0.1107 | 79.9 |
| Example 136 | 0.0465 | 97.24 |
| Example 137 | 0.1288 | 81.87 |
| Example 138 | 0.0856 | 91.15 |
| Example 139 | 0.129 | 81.9 |
| Example 140 | 0.1916 | 107.4 |
| Example 141 | 0.0783 | 113 |
| Example 142 | 0.0258 | 111.9 |
| Example 143 | 0.0277 | 104.2 |
| Example 144 | 0.1442 | 114.5 |
| Example 145 | 0.1558 | 105.9 |
| Example 146 | 0.0753 | 114.6 |
| Example 147 | 0.0948 | 98.83 |
| Example 148 | 0.0584 | 71.26 |
| Example 149 | 0.0546 | 100.3 |
| Example 150 | 0.0443 | 90.4 |
| Example 151 | 0.0365 | 92.52 |
| Example 152 | 0.019 | 95.22 |
| Example 153 | 0.0157 | 77.18 |
| Example 154 | 0.1412 | 93.66 |
| Example 155 | 0.1839 | 71.64 |
| Example 156 | 0.091 | 88.7 |
| Example 157 | 0.1232 | 87.55 |
| Example 158 | 0.1856 | 85.36 |
| Example 159 | 0.5014 | 72.42 |
| Example 160 | 0.2372 | 83.27 |
| Example 161 | 0.211 | 56.82 |
| Example 162 | 0.0834 | 99.52 |
| Example 163 | 0.1348 | 77.32 |
| Example 164 | 0.1455 | 107.1 |
| Example 165 | 0.4564 | 77.53 |
| Example 166 | 0.1313 | 99.39 |
| Example 167 | 0.0658 | 95.53 |
| Example 168 | 0.103 | 95.99 |
| Example 169 | 0.1852 | 82.95 |
| Example 170 | 0.082 | 101.1 |
| Example 171 | 0.0772 | 82.29 |
| Example 172 | 0.0619 | 84.13 |
| Example 173 | 0.1517 | 67.16 |
| Example 174 | 0.106 | 94.18 |
| Example 175 | 0.0712 | 97.24 |
| Example 176 | 0.4554 | 95.81 |
| Example 177 | 0.2093 | 79.05 |
| Example 179 | 0.0561 | 89.72 |
| Example 182 | 0.0152 | 80.24 |
| Example 183 | 0.0472 | 79.73 |
| Example 184 | 0.6381 | 59.64 |
| Example 185 | 0.7388 | 65.95 |
| Example 190 | 0.2248 | 102.2 |
| Example 191 | 0.0856 | 91.2 |
| Example 192 | 0.106 | 59.1 |
| Example 193 | 0.1609 | 84.94 |
| Example 194 | 0.0774 | 112.8 |
| Example 195 | 0.0184 | 112 |
| Example 196 | 0.0476 | 98.87 |
| Example 197 | 0.153 | 94.5 |
| Example 198 | 0.1 | 73.99 |
| Example 199 | 0.1087 | 91.37 |
| Example 200 | 0.6213 | 80.15 |
| Example 201 | 0.0279 | 103.2 |
| Example 202 | 0.0917 | 108.7 |
| Example 203 | 0.0353 | 113.6 |
| Example 204 | 0.2818 | 121.9 |
| Example 205 | 0.0592 | 119.6 |
| Example 206 | 0.05 | 98.74 |
| Example 207 | 0.0333 | 99.93 |
| Example 208 | 0.0333 | 104 |
| Example 209 | 0.0232 | 118.3 |
| Example 210 | 0.3183 | 87.37 |
| Example 211 | 0.2218 | 80.74 |
| Example 212 | 0.0238 | 107.7 |
| Example 213 | 0.0365 | 107.3 |
| Example 214 | 0.2544 | 102 |
| Example 215 | 0.0252 | 93.42 |
| Example 216 | 0.0743 | 103 |
| Example 217 | 0.0602 | 114.6 |
| Example 218 | 0.1925 | 115.9 |
| Example 219 | 0.0442 | 106.4 |
| Example 220 | 0.0677 | 84.66 |
| Example 221 | 0.0428 | 105.4 |
| Example 222 | 0.1365 | 115.1 |
| Example 223 | 0.045 | 106.7 |
| Example 224 | 0.0436 | 121.1 |
| Example 225 | 0.0503 | 114.5 |
| Example 226 | 0.3559 | 114.1 |
| Example 227 | 0.0354 | 115.1 |
| Example 228 | 0.0404 | 115.9 |
| | 0.0415* | 112 |
| Example 229 | 0.0449 | 94.72 |
| Example 230 | 0.1832 | 118.4 |
| Example 231 | 0.0935 | 111.1 |
| Example 232 | 0.0645 | 114.1 |
| Example 233 | 0.4894 | 126.2 |
| Example 234 | 0.394 | 91.86 |
| Example 235 | 0.0566 | 114.4 |
| Example 236 | 0.1195 | 115.9 |
| Example 237 | 0.582 | 80.7 |
| Example 238 | 0.125 | 108 |
| Example 239 | 0.109 | 106 |
| Example 240 | 0.159 | 107 |
| Example 241 | 0.118 | 106 |
| Example 242 | 0.102 | 107 |
| Example 243 | 0.086 | 109 |
| Example 244 | 0.206 | 105 |
| Example 245 | 0.0542 | 103 |
| Example 246 | 0.261 | 96.7 |
| Example 247 | 0.252 | 103 |
| Example 248 | 0.113 | 103 |
| Example 249 | 0.53 | 98.1 |
| Example 250 | 0.398 | 92.4 |
| Example 251 | 0.111 | 116 |
| Example 252 | 0.107 | 105 |
| Example 253 | 0.0201 | 104 |
| | 0.0216* | 105 |
| Example 254 | 0.498 | 107 |
| Example 255 | 0.155 | 104 |
| Example 256 | 0.0317 | 95.5 |
| Example 257 | 0.161 | 99.3 |
| Example 258 | 0.0888 | 108 |
| Example 259 | 0.0461 | 96.7 |
| Example 260 | 0.469 | 113 |
| Example 261 | 0.216 | 68 |
| Example 262 | 0.647 | 110 |
| Example 263 | 0.172 | 98 |
| Example 264 | 0.204 | 94.6 |
| Example 265 | 0.134 | 114 |
| Example 266 | 0.191 | 92.3 |
| Example 267 | 0.481 | 83.5 |
| Example 268 | 0.352 | 102 |
| Example 269 | 0.129 | 103 |
| Example 270 | 0.138 | 103 |
| Example 271 | 0.0545 | 81.3 |
| Example 272 | 0.0934 | 91.3 |
| Example 273 | 0.457 | 112 |
| Example 274 | 0.35 | 93 |
| Example 275 | 0.0505 | 95.9 |
| Example 276 | 0.518 | 102 |
| Example 277 | 0.304 | 105 |
| Example 278 | 0.434 | 78.6 |
| Example 279 | 0.277 | 88.7 |
| Example 280 | 0.12 | 89.2 |
| Example 281 | 0.382 | 113 |
| Example 282 | 0.155 | 109 |
| Example 283 | 0.441 | 78.6 |
| Example 284 | 0.452 | 72.7 |
| Example 285 | 0.217 | 98.4 |
| Example 286 | 0.246 | 105 |
| Example 287 | 0.0452 | 103 |
| Example 288 | 0.187 | 93.9 |
| Example 289 | 0.319 | 91.8 |
| Example 290 | 0.594 | 106 |
| Example 291 | 0.988 | 82.8 |
| Example 292 | 0.285 | 86.6 |
| Example 293 | 0.858 | 67.5 |
| Example 294 | 0.15 | 104 |
| Example 295 | 0.456 | 104 |
| Example 296 | 0.111 | 92.2 |
| Example 297 | 0.103 | 94.7 |
| Example 298 | 0.0196 | 108 |
| Example 299 | 0.0312 | 104 |
| Example 300 | 0.099 | 93.2 |
| Example 301 | 0.131 | 119 |
| Example 302 | 0.119 | 104 |
| Example 303 | 0.0883 | 118 |
| Example 304 | 0.114 | 111 |
| Example 305 | 0.104 | 99.9 |
| Example 306 | 0.241 | 104 |
| Example 307 | 0.0728 | 94.6 |
| Example 308 | 0.145 | 95.5 |
| Example 309 | 0.0504 | 108 |
| Example 310 | 0.153 | 107 |
| Example 311 | 0.238 | 105 |
| Example 312 | 0.215 | 102 |
| Example 313 | 0.196 | 92.3 |
| Example 314 | 0.0463 | 94.9 |
| Example 315 | 0.241 | 108 |
| Example 316 | 0.2 | 110 |
| Example 317 | 0.169 | 109 |
| Example 318 | 0.185 | 95.2 |
| Example 319 | 0.157 | 102 |
| Example 320 | 0.12 | 99.6 |
| Example 321 | 0.0734 | 100 |
| Example 322 | 0.231 | 104 |
| Example 323 | 0.0505 | 90.8 |
| Example 324 | 0.0989 | 110 |
| Example 325 | 0.151 | 111 |
| Example 326 | 0.174 | 106 |
| Example 327 | 0.0369 | 10 |
| Example 328 | 0.162 | 91.5 |
| Example 329 | 0.232 | 98.5 |
| Example 330 | 0.0206 | 108 |
| Example 331 | 0.167 | 93.3 |
| Example 332 | 0.0429 | 94.1 |
| Example 333 | 0.0318 | 101 |
| Example 334 | 0.0233 | 96.8 |
| Example 335 | 0.0142 | 113 |
| Example 336 | 0.0285 | 106 |
| Example 337 | 0.0855 | 95.9 |
| Example 338 | 0.0244 | 96.4 |
| Example 339 | 0.0678 | 110 |
| Example 340 | 0.0293 | 111 |
| Example 341 | 0.109 | 107 |
| Example 342 | 0.0844 | 104 |
| Example 343 | 0.0976 | 98.2 |
| Example 344 | 0.0315 | 107 |
| Example 345 | 0.0114 | 113 |
| Example 346 | 0.0423 | 96.7 |
| Example 347 | 0.0245 | 104 |
| Example 348 | 0.0516 | 94.3 |
| Example 349 | 0.0453 | 92.5 |
| Example 350 | 0.0367 | 94.9 |
| Example 351 | 0.0939 | 103 |
| Example 352 | 0.0275 | 117 |
| Example 353 | 0.0988 | 105 |
| Example 354 | 0.0233 | 106 |
| Example 355 | 0.0394 | 101 |
| Example 356 | 0.053 | 95.5 |
| Example 357 | 0.0771 | 100 |
| Example 358 | 0.077 | 98.4 |
| Example 359 | 0.0242 | 112 |
| Example 360 | 0.0714 | 113 |
| Example 361 | 0.0794 | 93.2 |
| Example 362 | 0.042 | 105 |
| Example 363 | 0.128 | 105 |
| Example 364 | 0.035 | 114 |

*indicates data from repeat experiments

All Example compounds of formula (Ia) tested exhibited improved PKM2-modulatory activity compared with mitapivat, as demonstrated by their lower EC₅₀ and/or higher Eₘₐₓ values for PKM2 activation.

### Biological Example 2—Human PKLR Activation Assay

### Measuring In Vitro Activation of Recombinant Human PKLR

Compound activation of recombinant human PKLR pyruvate kinase activity was determined by biochemical assay. N-terminal His-tagged enzyme was sourced from R&D Systems and its substrates phosphoenolpyruvate (PEP) and ADP from Sigma-Aldrich and 2Bscientific Ltd, respectively. The Kinase-Glo® Plus luminescence assay was from Promega. All other reagents were from Sigma-Aldrich. Test Compounds were prepared as 10 mM DMSO stocks and dilution series prepared in DMSO for direct dilution into Assay Buffer comprising 50 mM imidazole, 50 mM KCl, 7 mM MgCl₂, 0.01% Tween20, 0.05% BSA (pH 7.2).

### Assay Procedure

Human PKLR was diluted into Assay Buffer to a final concentration of 5 pM. Enzyme-Assay Buffer mix was dispensed into 384-well shallow-well white-walled plates and Test Compounds added by acoustic dispense (Echo®, Labcyte Inc). Following 10 minutes' incubation at room temperature, the enzyme reaction was initiated by acoustic dispensing of ADP+PEP substrate to final concentrations of 254 μM ADP and 53 μM ADP.

After 60 minutes' incubation on an orbital shaker (300 rpm, 26° C.), enzyme activity was quantified by the luminescent detection of generated ATP. Kinase-Glo® Plus reagent was added to each well and the plates incubated for a further 15 minutes on an orbital shaker in the dark (300 rpm, 26° C.) before luminescence measurement on a plate reader (PHERAstar® FSX, BMG Labtech).

Percentage activation was calculated by normalising fluorescence signals to plate LOW (DMSO vehicle) and HIGH (5 μM TEPP-46) controls. EC₅₀ and Eₘₐₓ values were determined from 4-parameter logistic fits of compound concentration-response curves.

A number of Example compounds of formula (Ia) were tested and the results are shown in Table 2 below.

TABLE 2
PKLR EC₅₀ values (μM) and Eₘₐₓ values (%)

| | hPKLR | hPKLR |
|---|---|---|
| Compound | EC₅₀ (μM) | Eₘₐₓ (%) |
| Mitapivat | 0.0321 | 107.2 |
| Example 1 | 0.0047 | 159.2 |
| Example 5 | 0.0193 | 110.8 |
| Example 38 | 0.0053 | 149.1 |
| Example 41 | 0.0158 | 109 |
| Example 54 | 0.0031 | 142.1 |
| Example 56 | 0.0101 | 131 |
| Example 60 | 0.0341 | 122.3 |
| Example 68 | 0.0171 | 133.9 |
| Example 73 | 0.014 | 116.7 |
| Example 75 | 0.0115 | 136.9 |
| Example 89 | 0.0096 | 135.2 |
| Example 90 | 0.0101 | 138.6 |
| Example 96 | 0.0093 | 127.9 |
| Example 110 | 0.0296 | 118.5 |
| Example 113 | 0.00964 | 118 |
| Example 128 | 0.0072 | 131.8 |
| Example 138 | 0.014 | 133.3 |
| Example 141 | 0.008 | 108.1 |
| Example 142 | 0.0041 | 107.8 |
| Example 150 | 0.0064 | 85.39 |
| Example 191 | 0.014 | 133 |
| Example 192 | 0.241 | 129 |
| Example 193 | 0.0863 | 111.1 |
| Example 194 | 0.1057 | 111.5 |
| Example 195 | 0.002 | 120.6 |
| Example 196 | 0.017 | 120.1 |
| Example 201 | 0.0029 | 139.9 |
| Example 202 | 0.0079 | 124.6 |
| Example 203 | 0.0024 | 123.2 |
| Example 205 | 0.008 | 115.3 |
| Example 206 | 0.0103 | 128 |
| Example 207 | 0.0042 | 123 |
| Example 219 | 0.0102 | 124.1 |
| Example 221 | 0.0034 | 134.1 |
| Example 223 | 0.0049 | 131.5 |
| Example 225 | 0.0086 | 117.4 |
| Example 228 | 0.0212 | 119.1 |
| Example 238 | 0.0144 | 127 |
| Example 270 | 0.108 | 136 |
| Example 239 | 0.0248 | 135 |
| Example 240 | 0.0303 | 125 |
| Example 241 | 0.0275 | 129 |
| Example 242 | 0.0222 | 134 |
| Example 244 | 0.0274 | 123 |
| Example 251 | 0.0113 | 111 |
| Example 252 | 0.018 | 120 |
| Example 253 | 0.00559 | 107 |
| | 0.00432* | 118 |
| Example 256 | 0.00333 | 136 |
| Example 258 | 0.0153 | 142 |
| Example 259 | 0.0131 | 123 |
| Example 265 | 0.0135 | 127 |
| Example 271 | 0.0264 | 119 |
| Example 272 | 0.0391 | 124 |
| Example 275 | 0.0195 | 120 |
| Example 287 | 0.013 | 110 |
| Example 304 | 0.016 | 119 |
| Example 305 | 0.0331 | 123 |
| Example 309 | 0.00658 | 118 |
| Example 314 | 0.00427 | 114 |
| Example 298 | 0.00265 | 118 |
| Example 320 | 0.0148 | 131 |
| Example 321 | 0.00657 | 134 |
| Example 323 | 0.00644 | 122 |
| Example 324 | 0.0233 | 121 |
| Example 327 | 0.00371 | 124 |

*indicates data from repeat experiments
Example compounds of formula (Ia) that were tested in this assay exhibited improved PKLR-modulatory activity compared with mitapivat, as demonstrated by their lower EC₅₀ and/or higher Eₘₐₓ values for PKLR activation.

### Biological Example 3—A549 Proliferation and Cytotoxicity Assay

### Induction of Serine Auxotrophy as a Cell-Based Readout of PKM2 Activation

In highly proliferative, glycolytically active cells, PKM2 activation accelerates flux through glycolysis and induces a powerful dependence of cell proliferation on the non-essential amino acid serine (Kung et al., 2012). The effect of compounds of Formula (Ia) on A549 cell proliferation in low-serine medium was assessed using a luminescence assay of viable cell number (CellTiter-Glo® 2.0, Promega) duplexed with a fluorescent cytotoxicity assessment (CelITox™ Green, Promega). Cell culture reagents were from ThermoFisher unless specified otherwise. Assay reagents were from Sigma-Aldrich unless specified otherwise. Test Compounds were prepared as 10 mM DMSO stocks and dilution series prepared in DMSO for direct dilution into culture medium.

### Assay Procedure

A549 cells were maintained in T225 flasks in a humidified incubator (37° C., 5% CO₂), in complete growth medium comprising RPMI 1640 supplemented with 10% heat-inactivated FBS plus 2 mM L-glutamine+1% penicillin/streptomycin (Sigma-Aldrich). Cells were passaged every 3-4 days at 80-90% confluence. The cell monolayer was washed twice with room-temperature Ca²⁺/Mg-free PBS and 4 ml room-temperature trypsin-EDTA added to the flask. After 5 minutes' incubation (37° C., 5% CO₂) to detach cells, trypsin was quenched with 13 ml warmed complete growth medium and the cells harvested and diluted to 2.4×10⁴ cells/ml for dispensing into 384-well clear-bottom black-walled plates (600 cells/well).

Following overnight incubation, growth medium was aspirated from each well and the cells washed twice with Assay Medium comprising Basal Medium Eagle supplemented with 3% heat-inactivated dialysed FBS plus 2 mM L-glutamine plus 20 μM L-serine (Sigma-Aldrich). After aspiration of the final wash medium, Assay Medium containing Test Compound was added to each well. DMSO vehicle controls were included on each plate.

Cells were incubated for 3 days in a humidified incubator (37° C., 5% CO₂) at which point CelITox™ Green and CellTiter-Glo® reagents were prepared according to the manufacturer's instructions. Triton-X was added to HIGH wells for cytotoxicity determination and the plate incubated for 30 minutes (20° C., ambient humidity and pCO₂). CelITox™ Green reagent was added to each well and the plate covered, placed on an orbital shaker @450 rpm for 2 minutes, and incubated in the dark for 15 minutes (20° C., ambient humidity and pCO₂). Endpoint fluorescence was measured using a plate reader (PHERAstar® FSX, BMG Labtech).

Following cytotoxicity determination, CellTiter-Glo® reagent was added to each well and the plate placed on an orbital shaker @450 rpm for 2 minutes to lyse cells. Plates were incubated in the dark for 15-30 minutes (20° C., ambient humidity and pCO₂) prior to endpoint luminescence measurement (PHERAstar® FSX, BMG Labtech).

Cytotoxicity was calculated by normalising fluorescence signals to plate LOW (DMSO vehicle) and HIGH (Triton X-treated) controls. CC₅₀ values were determined from a 4-parameter logistic fits of the compound concentration-response curve.

Proliferation was calculated by normalizing luminescence signals to LOW controls obtained from a parallel plate processed at the time of Test Compound challenge and HIGH (DMSO vehicle) wells from the compound plate. IC₅₀ values were determined from a 4-parameter logistic fit of compound concentration-response curves.

A number of Example compounds of formula (Ia) were tested and the results are shown in Table 3 below.

TABLE 3
A549 Cell Proliferation and Cytotoxicity

| Compound | Proliferation IC₅₀ (μM) |
|---|---|
| Mitapivat | 0.0044 |
| Example 1 | 0.0012 |
| Example 2 | 0.0008 |
| Example 3 | 0.0217 |
| Example 4 | 0.0007 |
| Example 6 | 0.0017 |
| Example 7 | 0.0067 |
| Example 8 | 0.2175 |
| Example 9 | 0.0175 |
| Example 10 | 0.0078 |
| Example 11 | 0.0039 |
| Example 12 | 0.0027 |
| Example 13 | 0.0031 |
| Example 15 | 0.0158 |
| Example 17 | 0.0029 |
| Example 19 | 0.0042 |
| Example 20 | 0.0064 |
| Example 21 | 0.0121 |
| Example 24 | 0.003 |
| Example 29 | 0.0008 |
| Example 30 | 0.0158 |
| Example 31 | 0.042 |
| Example 32 | 0.0032 |
| Example 33 | 0.0017 |
| Example 34 | 0.0648 |
| Example 35 | 0.0104 |
| Example 36 | 0.0206 |
| Example 37 | 0.0036 |
| Example 38 | 0.0015 |
| Example 39 | 0.0074 |
| Example 40 | 0.0037 |
| Example 41 | 0.002 |
| Example 42 | 0.0015 |
| Example 43 | 0.0017 |
| Example 44 | 0.0418 |
| Example 45 | 0.0262 |
| Example 46 | 0.0048 |
| Example 47 | 0.0005 |
| Example 48 | 0.0016 |
| Example 50 | 0.0031 |
| Example 51 | 0.0035 |
| Example 52 | 0.0019 |
| Example 53 | 0.0057 |
| Example 54 | 0.0022 |
| Example 55 | 0.0217 |
| Example 56 | 0.0017 |
| Example 57 | 0.0023 |
| Example 58 | 0.0418 |
| Example 59 | 0.0032 |
| Example 60 | 0.0036 |
| Example 61 | 0.0158 |
| Example 62 | 0.0025 |
| Example 63 | 0.0043 |
| Example 64 | 0.0021 |
| Example 65 | 0.0174 |
| Example 66 | 0.0043 |
| Example 67 | 0.0033 |
| Example 68 | 0.0022 |
| Example 70 | 0.0411 |
| Example 71 | 0.0141 |
| Example 72 | 0.0032 |
| Example 73 | 0.0035 |
| Example 74 | 0.0387 |
| Example 75 | 0.0022 |
| Example 76 | 0.0041 |
| Example 77 | 0.004 |
| Example 78 | 0.0891 |
| Example 79 | 0.0194 |
| Example 81 | 0.0067 |
| Example 82 | 0.0035 |
| Example 89 | 0.0017 |
| Example 90 | 0.003 |
| Example 95 | 0.0099 |
| Example 96 | 0.0033 |
| Example 98 | 0.009 |
| Example 99 | 0.0046 |
| Example 100 | 0.0739 |
| Example 101 | 0.3542 |
| Example 102 | 0.0022 |
| Example 103 | 0.0007 |
| Example 104 | 0.0012 |
| Example 106 | 0.0062 |
| Example 108 | 0.002 |

Example compounds of formula (Ia) that were tested in this assay exhibited acceptable or good anti-proliferative activity, as demonstrated by their IC₅₀ values. Certain compounds of formula (Ia) exhibited improved anti-proliferative activity compared with mitapivat, as demonstrated by their lower IC₅₀ values.

### REFERENCES

Abulizi et al. Cell Metab. 2020, 32(5):751-766.e11.
Alves-Filho et al. Front Immunol. 2016, 7(145), 1-7.
Barazzoni et al. Eating and Weight Disorders—Studies on Anorexia, Bulimia and Obesity 2018, 23, 149-157.
Bettaieb et al. The Journal of Biological Chemistry 2013, 288(24), 17360-17371.
Bianchi et al. Haematologica 2020, 105(9), 2218-2228.
Cangçado et al., Hematology, Transfusion and Cell Therapy, 2018, 40(1), 1-2.
Chhipa et al. Life Sciences 2018, 280, DOI: 10.1016/j.Ifs.2021.119694.
Dong et al. Oncol Lett. 2016, 11(3), 1980-1986.
Grace et al. N. Engl. J. Med. 2019, 381(10), 933-944
Kung et al. Chemistry & Biology 2012, 19, 1187-1198
Kung et al. Blood 2017, 14; 130(11), 1347-1356.
Lewandowski et al. Cell Metab. 2020, 32(5):736-750.e5.
Liu et al. J. Diabetes Investig. 2020, 12(5):697-709.
Pålsson-McDermott et al. Cell Research 2020, 30:300-314.
Puckett et al. International Journal of Molecular Sciences 2021, 22, 1171.
Qi et al. Nat Med. 2017, 23(6), 753-762
Yi et al. Front. Immunol. 2021, DOI: 10.3389/fimmu.2020.595316.

### Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application, of which this description and claims form part, may be used as a basis for priority in respect of any subsequent application. The claims of such subsequent application may be directed to any feature or combination of features described herein. They may take the form of product, composition, process, or use claims and may include, by way of example and without limitation, the following claims.

1-114. (canceled)

115: A compound of formula (Ia): 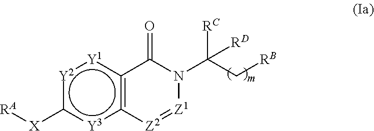
wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available atom by one or more R^{1A}, wherein each R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A} NHCOCH₃, CO₂H, CH₂CO₂H, CONR^{2A}R^{3A}, CH₂CONR^{2A}R^{3A}, C₃₋₆ cycloalkyl, oxo, and a 4 membered heterocyclic ring, wherein the 4 membered heterocyclic ring is optionally substituted by hydroxy;
R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A};
wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
or
Y² is absent and one of Y¹ and Y³ is S, O or NR^{5A}, and the remaining Y¹ or Y³ is N or CR^{6A};
wherein R^{5A} is H or C₁₋₃ alkyl; and
R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A};
wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available atom by one or more R^{1B}, wherein each R^{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl, oxo, and a 4 membered heterocyclic ring, wherein the 4 membered heterocyclic ring is optionally substituted by hydroxy;
R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —C(R^{E})(R^{F})O— or CH₂NH—; and
R^{E} and R^{F} are H, or R^{E} and R^{F} join to form a C₃₋₅ heterocycloalkyl ring;
or a pharmaceutically acceptable salt and/or solvate thereof.

116: A compound of formula (Ia) according to claim 115, which is a compound of formula (Ib): 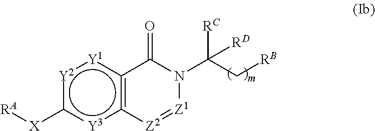
wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available carbon or nitrogen atom by one or more R^{1A} wherein each R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A} NHCOCH₃, CO₂H, CH₂CO₂H, CONR^{2A}R^{3A}, CH₂CONR^{2A}R^{3A}, C₃₋₆ cycloalkyl and oxo;
R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A};
wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
or
Y² is absent and one of Y¹ and Y³ is S, O or NR^{5A}, and the remaining Y¹ or Y³ is N or CR^{6A};
wherein R^{5A} is H or C₁₋₃ alkyl; and
R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A};
wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available carbon or nitrogen atom by one or more R^{1B}, wherein each R^{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl and oxo;
R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —CH₂O— or CH₂NH—;
or a pharmaceutically acceptable salt and/or solvate thereof.

117: A compound of formula (Ia) according to claim 115, which is a compound of formula (I): 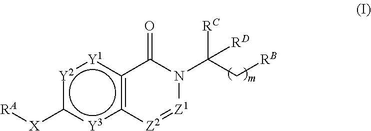
wherein,
R^{A} is phenyl, phenyl fused to a 5-7 membered heterocyclic ring, 5-10 membered heteroaryl, or 5-6 membered heteroaryl fused to a 5-7 membered heterocyclic ring; wherein R^{A} is optionally substituted on an available carbon atom by one or more R^{1A}, wherein R^{1A} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A}, C₁₋₆ alkyl-NR^{2A}R^{3A} NHCOCH₃, CO₂H, CH₂CO₂H, CONR^{2A}R^{3A}, CH₂CONR^{2A}R^{3A}, C₃₋₆ cycloalkyl and oxo;
R^{2A} and R^{3A} are independently selected from the group consisting of H and C₁₋₆ alkyl, or R^{2A} and R^{3A} together with the N atom to which they are attached combine to form a 4-7 membered heterocyclic ring which is optionally substituted on an available atom by one or more groups selected from C₁₋₂ alkyl and oxo;
Y¹, Y² and Y³ are independently selected from the group consisting of CR^{4A} and N, wherein at least two of Y¹, Y² and Y³ are CR^{4A};
wherein R^{4A} is selected from the group consisting of H, C₁₋₃ alkyl, C₂₋₃ alkenyl, C₂₋₃ alkynyl, OR^{8A}, halo, cyano, methanesulfonyl, CF₃ and NR^{8A}R^{9A};
wherein R^{8A} and R^{9A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{10A}R^{11A};
wherein R^{10A} and R^{11A} are independently selected from the group consisting of H and C₁₋₆ alkyl, or R^{10A} and R^{11A} together with the N atom to which they are attached combine to form a 4-7 membered heterocyclic ring which is optionally substituted on an available atom by one or more groups selected from C₁₋₂ alkyl and oxo;
or
Y² is absent and one of Y¹ and Y³ is S, O or NR^{5A}, and the remaining Y¹ or Y³ is N or CR^{6A};
wherein R^{5A} is H or C₁₋₃ alkyl; and
R^{6A} is selected from the group consisting of H, C₁₋₃ alkyl, C₂₋₃ alkenyl, C₂₋₃ alkynyl, OR^{12A}, halo, cyano, methanesulfonyl, CF₃ and NR^{12A}R^{13A};
wherein R^{12A} and R^{13A} are independently selected from the group consisting of H and C₁₋₆ alkyl, wherein C₁₋₆ alkyl is optionally substituted on an available carbon atom by one or more groups selected from halo, hydroxy, methoxy, CO₂H and NR^{14A}R^{15A};
wherein R^{14A} and R^{15A} are independently selected from the group consisting of H and C₁₋₆ alkyl, or R^{14A} and R^{15A} together with the N atom to which they are attached combine to form a 4-7 membered heterocyclic ring which is optionally substituted on an available atom by one or more groups selected from C₁₋₂ alkyl and oxo;
Z¹ and Z² are independently selected from N and CH, wherein at least one of Z¹ and Z² is CH;
R^{B} is phenyl, phenyl fused to a 5-7 membered heterocyclic ring, 5-10 membered heteroaryl, 5-6 membered heteroaryl fused to a 5-7 membered heterocyclic ring, 5-7 membered heterocyclyl or C₃₋₆ cycloalkyl; wherein R^{B} is optionally substituted on an available carbon atom by one or more R^{1B}, wherein R^{1B} is independently selected from the group consisting of halo, C₁₋₆ alkyl, C₂₋₆ alkenyl, C₂₋₆ alkynyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2B}R^{3B}, C₁₋₆ alkyl-NR^{2B}R^{3B}, NHCOCH₃, CO₂H, CH₂CO₂H, CONR^{2B}R^{3B}, CH₂CONR^{2B}R^{3B}, C₃₋₆ cycloalkyl and oxo;
R^{2B} and R^{3B} are independently selected from the group consisting of H and C₁₋₆ alkyl, or R^{2B} and R^{3B} together with the N atom to which they are attached combine to form a 4-7 membered heterocyclic ring which is optionally substituted on an available atom by one or more groups selected from C₁₋₂ alkyl and oxo;
R^{C} is H, fluoro, C₁₋₃ alkyl, C₁₋₂ hydroxyalkyl or C₁₋₂ methoxyalkyl;
R^{D} is H or fluoro;
m is 0 or 1;
X is S(═O)₁₋₂, —CH₂S(═O)₁₋₂—, S(═O)(═NH), —S(═O)₂NH—, —NHS(═O)₂—, CH₂, —CH₂O— or CH₂NH—;
or a pharmaceutically acceptable salt and/or solvate thereof.

118: The compound, pharmaceutically acceptable salt and/or solvate thereof according to claim 115, wherein R^{A} is phenyl, phenyl fused to a 5-6 membered heterocyclic ring, 5-6 membered heteroaryl, or 5-6 membered heteroaryl fused to a 5-6 membered heterocyclic ring, and is optionally substituted on an available atom by one or more R^{1A}.

119: The compound, pharmaceutically acceptable salt and/or solvate thereof according to claim 115, wherein R^{A} is phenyl optionally substituted on an available carbon atom by one or more R^{1A}.

120: The compound, pharmaceutically acceptable salt and/or solvate thereof according to claim 115, wherein R^{A} is 5-10 membered heteroaryl, such as selected from the group consisting of pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl and quinazolinyl; and is optionally substituted on an available atom by one or more R^{1A}.

121: The compound, pharmaceutically acceptable salt and/or solvate thereof according to claim 115, wherein each R^{1A} is independently selected from the group consisting of halo, C₁₋₆ alkyl, C₁₋₆ hydroxyalkyl, C₁₋₆ methoxyalkyl, C₁₋₆ haloalkyl, hydroxy, OC₁₋₆ alkyl, OC₁₋₆ hydroxyalkyl, OC₁₋₆ methoxyalkyl, OC₁₋₆ haloalkyl, cyano, NR^{2A}R^{3A} NHCOCH₃, CO₂H, CONR^{2A}R^{3A}, C₃₋₆ cycloalkyl, oxo, and a 4 membered heterocyclic ring, wherein the 4 membered heterocyclic ring is optionally substituted by hydroxy; and in particular is independently selected from halo, C₁₋₆ alkyl, C₁₋₆ hydroxyalkyl, C₁₋₆ methoxyalkyl, C₁₋₆ haloalkyl, hydroxy and OC₁₋₆ alkyl e.g. C₁₋₆ alkyl, OC₁₋₆ alkyl, oxo, and oxetane or azetidine either of which is optionally substituted by hydroxy.

122: The compound, pharmaceutically acceptable salt and/or solvate thereof according to claim 115, wherein R^{A} is unsubstituted.

123: The compound, pharmaceutically acceptable salt and/or solvate thereof according to claim 115, wherein R^{B} is selected from the group consisting of phenyl, phenyl fused to a 5-7 membered heterocyclic ring selected from the group consisting of 2,3-dihydrobenzofuranyl, indolinyl, 1,2,3,4-tetrahydroquinolinyl, benzo-1,4-dioxanyl, 1,3-benzodiazole and 3,4-dihydro-2H-1,4-benzoxazine, pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl, quinazolinyl, pyrrolidinyl, tetrahydrofuranyl, tetrahydrothienyl, tetrahydropyranyl, piperidinyl, piperazinyl, morpholinyl, thiomorpholinyl, pyridone and pyridazinone; and is optionally substituted on an available atom by one or more R^{1B}.

124: The compound, pharmaceutically acceptable salt and/or solvate thereof according to claim 115, wherein R^{B} is phenyl optionally substituted on an available carbon atom by one or more R^{1B}.

125: The compound, pharmaceutically acceptable salt and/or solvate thereof according to claim 115, wherein R^{B} is 5-10 membered heteroaryl such as selected from the group consisting of pyrrolyl, furanyl, thienyl, imidazolyl, pyrazolyl, thiazolyl, isothiazolyl, oxadiazolyl, thiadiazolyl, triazolyl, oxazolyl, isoxazolyl, tetrazolyl, pyridinyl, pyrimidinyl, pyradizinyl, pyrazinyl, furo[3,2-b]pyridinyl, pyrazolo[1,5-a]pyridinyl, imidazo[1,2-a]pyridinyl, indolyl, indazolyl, benzofuranyl, benzimidazolyl, benzothiazolyl, benzothiophenyl, quinolinyl, isoquinolinyl and quinazolinyl; and in particular is selected from the group consisting of furanyl, thienyl, pyrazolyl, thiazolyl, isothiazolyl, isoxazolyl, pyridinyl, pyrazinyl, pyrazolo[1,5-a]pyridinyl, imidazo[1,2-a]pyridinyl and indazolyl; and is optionally substituted on an available atom by one or more R^{1B}.

126: The compound, pharmaceutically acceptable salt and/or solvate thereof according to claim 115, wherein R^{B} is 5-6 membered heteroaryl fused to a 5-7 membered heterocyclic ring, selected group the group consisting of 2,3-dihydropyrazolo[5,1-b]oxazolyl, 2,3-dihydrofuro[2,3-b]pyridinyl, 2,3-dihydrofuro[3,2-b]pyridinyl, 2,3-dihydro-[1,4]dioxino[2,3-c]pyridinyl and 2,3-dihydro-[1,4]dioxino[2,3-b]pyridinyl; and is optionally substituted on an available atom (e.g. a carbon or nitrogen atom, in particular a carbon atom) by one or more R^{1B}.

127: The compound, pharmaceutically acceptable salt and/or solvate thereof according to claim 115, wherein each R^{1B} is independently selected from the group consisting of halo, C₁₋₆ alkyl, C₁₋₆ hydroxyalkyl, C₁₋₆ haloalkyl, hydroxy, OC₁₋₆ alkyl, OC₁₋₆ hydroxyalkyl, OC₁₋₆ haloalkyl, cyano, NR^{2B}R^{3B}, CONR^{2B}R^{3B}, CH₂CONR^{2B}R^{3B}, C₃₋₆ cycloalkyl, oxo, and a 4 membered heterocyclic ring, wherein the 4 membered heterocyclic ring is optionally substituted by hydroxy; and in particular is independently selected from the group consisting of fluoro, chloro, methyl, ethyl, CH₂OH, CH₂CH₂OH, CHF₂, hydroxy, OCH₃, OCH₂CH₂OH, OCHF₂, cyano, NH₂, CONH₂, CONHCH₃, CH₂CONH₂, cyclopropyl, oxo, and oxetane or azetidine either of which is optionally substituted by hydroxy.

128: The compound, pharmaceutically acceptable salt and/or solvate thereof according to claim 115, wherein R^{B} is unsubstituted.

129: The compound, pharmaceutically acceptable salt and/or solvate thereof according to claim 115, wherein R^{2B} and R^{3B} are independently selected from the group consisting of H and C₁₋₆ alkyl.

130: The compound, pharmaceutically acceptable salt and/or solvate thereof according to claim 115, wherein R^{2B} and R^{3B} are both H.

131: The compound, pharmaceutically acceptable salt and/or solvate thereof according to claim 115, wherein Y¹, Y² and Y³ are independently selected from the group consisting of CR^{4A} and N, wherein at least two of Y¹, Y² and Y³ are CR^{4A}; wherein R^{4A} is as defined in claim 1.

132: The compound, pharmaceutically acceptable salt and/or solvate thereof according to claim 131, wherein Y¹, Y² and Y³ are all CR^{4A}, wherein suitably R^{4A} is H.

133: The compound, pharmaceutically acceptable salt and/or solvate thereof according to claim 115, wherein Z¹ is N and Z² is CH.

134: The compound, pharmaceutically acceptable salt and/or solvate thereof according to claim 115, wherein R^{C} is H, fluoro, CH₃, CH₂OH or CH₂OCH₃, and in particular is H.

135: The compound, pharmaceutically acceptable salt and/or solvate thereof according to claim 115, wherein R^{D} is H or fluoro, and in particular is H.

136: The compound, pharmaceutically acceptable salt and/or solvate thereof according to claim 115, wherein m is 0.

137: The compound, pharmaceutically acceptable salt and/or solvate thereof according to claim 115, wherein X is S(═O)₂.

138: The compound, pharmaceutically acceptable salt and/or solvate thereof according to claim 115, wherein X is S(═O).

139: The compound, pharmaceutically acceptable salt and/or solvate thereof according to claim 115, wherein X is —CH₂S(═O)₁₋₂—.

140: The compound, pharmaceutically acceptable salt and/or solvate thereof according to claim 115, wherein X is S(═O)(═NH).

141: The compound, pharmaceutically acceptable salt and/or solvate thereof according to claim 115, wherein X is —S(═O)₂NH—.

142: The compound, pharmaceutically acceptable salt and/or solvate thereof according to claim 115, wherein X is —NHS(═O)₂—.

143: The compound, pharmaceutically acceptable salt and/or solvate thereof according to claim 115, wherein X is CH₂.

144: The compound, pharmaceutically acceptable salt and/or solvate thereof according to claim 115, wherein X is —C(R^{E})(R^{F})O— and R^{E} and R^{F} are H or R^{E} and R^{F} join to form a C₃₋₅ heterocycloalkyl ring, such as X is —CH₂O—.

145: The compound, pharmaceutically acceptable salt and/or solvate thereof according to claim 115, wherein X is CH₂NH—.

146: The compound, pharmaceutically acceptable salt and/or solvate thereof according to claim 115, wherein the compound of formula (Ia) is a compound of formula (IIa): 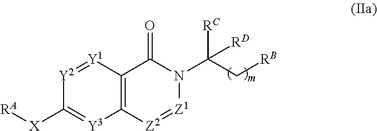
wherein Y¹, Y² and Y³ are independently selected from the group consisting of CR^{4A} and N, wherein at least two of Y¹, Y² and Y³ are CR^{4A};
or a pharmaceutically acceptable salt and/or solvate thereof.

147: The compound, pharmaceutically acceptable salt and/or solvate thereof according to claim 146, wherein the compound of formula (Ia) is a compound of formula (IIaA): 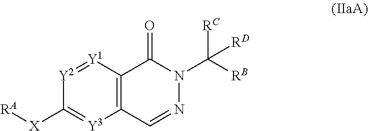
wherein Y¹, Y² and Y³ are independently selected from the group consisting of CR^{4A} and N, wherein at least two of Y¹, Y² and Y³ are CR^{4A};
or a pharmaceutically acceptable salt and/or solvate thereof.

148: The compound, pharmaceutically acceptable salt and/or solvate thereof according to claim 147, wherein the compound of formula (Ia) is a compound of formula (IIaB): 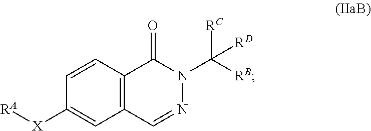
or a pharmaceutically acceptable salt and/or solvate thereof.

149: The compound, pharmaceutically acceptable salt and/or solvate thereof according to claim 148, wherein the compound of formula (Ia) is a compound of formula (IIaC): 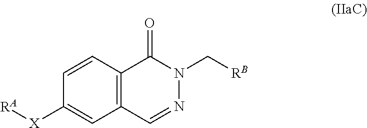
or a pharmaceutically acceptable salt and/or solvate thereof.

150: The compound, pharmaceutically acceptable salt and/or solvate thereof according to claim 149, wherein the compound of formula (Ia) is a compound of formula (IaD): 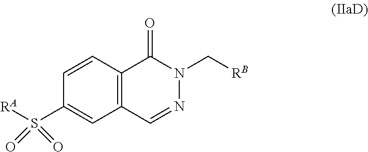
or a pharmaceutically acceptable salt and/or solvate thereof.

151: The compound, pharmaceutically acceptable salt and/or solvate thereof according to claim 115, selected from the group consisting of:
2-((1-cyclopropyl-1H-pyrazol-3-yl)methyl)-6-((4-methoxyphenyl)sulfonyl)phthalazin-1(2H)-one;
2-(indolin-4-ylmethyl)-6-((4-methoxyphenyl)sulfonyl)phthalazin-1(2H)-one;
2-((2-hydroxypyridin-4-yl)methyl)-6-((4-methoxyphenyl)sulfonyl)phthalazin-1(2H)-one;
6-((4-methoxyphenyl)sulfonyl)-2-((1-methyl-2-oxo-1,2-dihydropyridin-3-yl)methyl)phthalazin-1(2H)-one;
2-((1-(2-hydroxyethyl)-2-oxo-1,2-dihydropyridin-3-yl)methyl)-6-(4-methoxyphenylsulfonyl)phthalazin-1(2H)-one;
2-((1H-pyrazol-3-yl)methyl)-6-((4-methoxyphenyl)sulfonyl)phthalazin-1(2H)-one;
2-((5-hydroxypyridin-3-yl)methyl)-6-(phenylsulfonyl)phthalazin-1(2H)-one;
2-(2-methoxy-1-(6-methoxypyridin-3-yl)ethyl)-6-(phenylsulfonyl)phthalazin-1(2H)-one;
3-((1-oxo-6-(phenylsulfonyl)phthalazin-2(1H)-yl)methyl)picolinamide;
2-((1-(2-hydroxyethyl)-1H-pyrazol-3-yl)methyl)-6-(phenylsulfonyl)phthalazin-1(2H)-one;
2-((1-ethyl-1H-pyrazol-3-yl)methyl)-6-(phenylsulfonyl)phthalazin-1(2H)-one;
2-((4-methyl-1H-pyrazol-3-yl)methyl)-6-(phenylsulfonyl)phthalazin-1(2H)-one;
3-((1-oxo-6-(phenylsulfonyl)phthalazin-2(1H)-yl)methyl)thiophene-2-carboxamide;
6-methoxy-3-((1-oxo-6-(phenylsulfonyl)phthalazin-2(1H)-yl)methyl)picolinamide;
3-((1-oxo-6-(phenylsulfonyl)phthalazin-2(1H)-yl)methyl)-1H-pyrazole-4-carboxamide;
2-((1-(2-hydroxyethyl)-4-methyl-1H-pyrazol-3-yl)methyl)-6-(phenylsulfonyl)phthalazin-1(2H)-one;
3-((6-((4-methoxyphenyl)sulfonyl)-1-oxophthalazin-2(1H)-yl)methyl)-N-methylfuran-2-carboxamide;
3-((1-oxo-6-(phenylsulfonyl)phthalazin-2(1H)-yl)methyl)-1H-pyrazole-4-carbonitrile;
2-(2-hydroxy-1-phenylethyl)-6-(phenylsulfonyl)phthalazin-1(2H)-one;
2-(2-(hydroxymethyl)benzyl)-6-(phenylsulfonyl)phthalazin-1(2H)-one;
2-((6-(2-hydroxyethoxy)pyridin-3-yl)methyl)-6-(phenylsulfonyl)phthalazin-1(2H)-one;
2-((2-(hydroxymethyl)thiophen-3-yl)methyl)-6-(phenylsulfonyl)phthalazin-1(2H)-one;
3-((1-oxo-6-(phenylsulfonyl)phthalazin-2(1H)-yl)methyl)thiophene-2-carbonitrile;
2-((1-(2-hydroxyethyl)-1H-pyrazol-3-yl)methyl)-6-((4-methoxyphenyl)sulfonyl)phthalazin-1(2H)-one;
2-((5-cyclopropyl-1H-pyrazol-3-yl)methyl)-6-(phenylsulfonyl)phthalazin-1(2H)-one;
2-((4-(hydroxymethyl)-1H-pyrazol-3-yl)methyl)-6-(phenylsulfonyl)phthalazin-1(2H)-one;
2-((2,3-dihydropyrazolo[5,1-b]oxazol-6-yl)methyl)-6-(phenylsulfonyl)phthalazin-1(2H)-one;
2-((1-(difluoromethyl)-1H-pyrazol-3-yl)methyl)-6-(phenylsulfonyl)phthalazin-1(2H)-one;
2-((2,3-dihydrobenzo[b][1,4]dioxin-6-yl)methyl)-6-(phenylsulfonyl)phthalazin-1(2H)-one;
2-((2-methylthiazol-5-yl)methyl)-6-(phenylsulfonyl)phthalazin-1(2H)-one;
2-((5-methoxypyridin-3-yl)methyl)-6-(phenylsulfonyl)phthalazin-1(2H)-one;
2-(2-fluorobenzyl)-6-(phenylsulfonyl)phthalazin-1(2H)-one;
2-(2-fluoro-5-methoxybenzyl)-6-(phenylsulfonyl)phthalazin-1(2H)-one;
2-(imidazo[1,2-a]pyridin-5-ylmethyl)-6-(phenylsulfonyl)phthalazin-1(2H)-one;
2-((2,2-dimethyl-2,3-dihydrobenzofuran-5-yl)methyl)-6-(phenylsulfonyl)phthalazin-1(2H)-one;
2-((1-methyl-1H-indazol-5-yl)methyl)-6-(phenylsulfonyl)phthalazin-1(2H)-one;
2-((6-methoxypyridin-2-yl)methyl)-6-(phenylsulfonyl)phthalazin-1(2H)-one;
2-((6-aminopyridin-2-yl)methyl)-6-(phenylsulfonyl)phthalazin-1(2H)-one;
2-((1-methyl-1H-indazol-6-yl)methyl)-6-(phenylsulfonyl)phthalazin-1(2H)-one;
2-(3-(difluoromethoxy)benzyl)-6-((4-methoxyphenyl)sulfonyl)phthalazin-1(2H)-one;
2-((6-methoxypyridin-3-yl)methyl)-6-(phenylsulfonyl)phthalazin-1(2H)-one;
6-((4-methoxyphenyl)sulfonyl)-2-(pyridin-3-ylmethyl)phthalazin-1(2H)-one;
2-(2-aminobenzyl)-6-((4-methoxyphenyl)sulfonyl)phthalazin-1(2H)-one;
6-((4-methoxyphenyl)sulfonyl)-2-phenethylphthalazin-1(2H)-one;
2-(isoxazol-5-ylmethyl)-6-((4-methoxyphenyl)sulfonyl)phthalazin-1(2H)-one;
2-(4-chloro-2-fluorobenzyl)-6-((4-methoxyphenyl)sulfonyl)phthalazin-1(2H)-one;
2-((2,3-dihydrobenzofuran-5-yl)methyl)-6-((4-methoxyphenyl)sulfonyl)phthalazin-1(2H)-one;
2-((1H-indazol-3-yl)methyl)-6-((4-methoxyphenyl)sulfonyl)phthalazin-1(2H)-one;
2-(4-methoxybenzyl)-6-(phenylsulfonyl)phthalazin-1(2H)-one;
2-benzyl-6-((4-methoxyphenyl)sulfonyl)phthalazin-1(2H)-one;
6-((4-methoxyphenyl)sulfonyl)-2-((6-methoxypyridin-3-yl)methyl)phthalazin-1(2H)-one;
2-(2-fluoro-3-methoxybenzyl)-6-((4-methoxyphenyl)sulfonyl)phthalazin-1(2H)-one;
2-(2,6-difluoro-4-methoxybenzyl)-6-((4-methoxyphenyl)sulfonyl)phthalazin-1(2H)-one;
6-((4-methoxyphenyl)sulfonyl)-2-((5-methyl-1H-pyrazol-3-yl)methyl)phthalazin-1(2H)-one;
2-((2-hydroxypyridin-4-yl)methyl)-6-((4-methoxyphenyl)sulfonyl)phthalazin-1(2H)-one;
2-((2-hydroxypyridin-3-yl)methyl)-6-((4-methoxyphenyl)sulfonyl)phthalazin-1(2H)-one;
2-(benzo[d][1,3]dioxol-5-ylmethyl)-6-(phenylsulfonyl)phthalazin-1(2H)-one;
2-((1H-indazol-6-yl)methyl)-6-(phenylsulfonyl)phthalazin-1(2H)-one;
2-((5-methoxypyridin-2-yl)methyl)-6-(phenylsulfonyl)phthalazin-1(2H)-one;
2-((1-methyl-1H-pyrazol-3-yl)methyl)-6-(phenylsulfonyl)phthalazin-1(2H)-one;
2-(2-((1-oxo-6-(phenylsulfonyl)phthalazin-2(1H)-yl)methyl)phenyl)acetamide;
2-((6-(difluoromethoxy)pyridin-3-yl)methyl)-6-(phenylsulfonyl)phthalazin-1(2H)-one;
2-(2-(2-hydroxyethyl)benzyl)-6-(phenylsulfonyl)phthalazin-1(2H)-one;
2-((2-methyl-2,3-dihydrobenzofuran-5-yl)methyl)-6-(phenylsulfonyl)phthalazin-1(2H)-one;
6-(phenylsulfonyl)-2-(pyridin-3-ylmethyl)phthalazin-1(2H)-one;
2-((1-oxo-6-(phenylsulfonyl)phthalazin-2(1H)-yl)methyl)benzamide;
2-(2-methoxy-1-phenylethyl)-6-(phenylsulfonyl)phthalazin-1(2H)-one;
2-((1H-pyrazol-3-yl)methyl)-6-(phenylsulfonyl)phthalazin-1(2H)-one;
6-(phenylsulfonyl)-2-(1-(pyridin-3-yl)ethyl)phthalazin-1(2H)-one;
2-((1,3-dimethyl-1H-pyrazol-4-yl)methyl)-6-(phenylsulfonyl)phthalazin-1(2H)-one;
2-benzyl-6-(phenylsulfonyl)phthalazin-1(2H)-one;
6-((4-methoxyphenyl)sulfonyl)-2-((1-methyl-1H-pyrazol-3-yl)methyl)phthalazin-1(2H)-one;
2-((1-cyclopropyl-1H-pyrazol-3-yl)methyl)-6-(phenylsulfonyl)phthalazin-1(2H)-one;
2-(1-(6-methoxypyridin-3-yl)ethyl)-6-(phenylsulfonyl)phthalazin-1(2H)-one;
2-((6-methylpyridin-2-yl)methyl)-6-(phenylsulfonyl)phthalazin-1(2H)-one;
2-((4-chloro-1-methyl-1H-pyrazol-3-yl)methyl)-6-(phenylsulfonyl)phthalazin-1(2H)-one;
2-((2-methylthiazol-4-yl)methyl)-6-(phenylsulfonyl)phthalazin-1(2H)-one;
2-(difluoro(pyridin-3-yl)methyl)-6-(phenylsulfonyl)phthalazin-1(2H)-one;
2-(2-methoxybenzyl)-6-(phenylsulfonyl)phthalazin-1(2H)-one;
2-((5-methylisoxazol-3-yl)methyl)-6-(phenylsulfonyl)phthalazin-1(2H)-one;
2-(3-methoxybenzyl)-6-(phenylsulfonyl)phthalazin-1(2H)-one;
2-(2-methoxy-1-phenylethyl)-6-((4-methoxyphenyl)sulfonyl)phthalazin-1(2H)-one;
2-((1-ethyl-1H-pyrazol-5-yl)methyl)-6-(phenylsulfonyl)phthalazin-1(2H)-one;
3-((1-oxo-6-(phenylsulfonyl)phthalazin-2(1H)-yl)methyl)benzamide;
3-((1-oxo-6-(phenylsulfonyl)phthalazin-2(1H)-yl)methyl)furan-2-carboxamide;
5-((1-oxo-6-(phenylsulfonyl)phthalazin-2(1H)-yl)methyl)furan-2-carboxamide;
2-((5-methoxypyrazin-2-yl)methyl)-6-(phenylsulfonyl)phthalazin-1(2H)-one;
2-((2-methoxypyridin-3-yl)methyl)-6-(phenylsulfonyl)phthalazin-1(2H)-one;
2-((1,4-dimethyl-1H-pyrazol-3-yl)methyl)-6-(phenylsulfonyl)phthalazin-1(2H)-one;
2-((1,5-dimethyl-1H-pyrazol-3-yl)methyl)-6-(phenylsulfonyl)phthalazin-1(2H)-one;
2-chloro-6-((1-oxo-6-(phenylsulfonyl)phthalazin-2(1H)-yl)methyl)benzamide;
2-methoxy-6-((1-oxo-6-(phenylsulfonyl)phthalazin-2(1H)-yl)methyl)benzamide;
4-((1-oxo-6-(phenylsulfonyl)phthalazin-2(1H)-yl)methyl)thiazole-2-carboxamide;
5-methyl-4-((1-oxo-6-(phenylsulfonyl)phthalazin-2(1H)-yl)methyl) isoxazole-3-carboxamide;
2-((1-isopropyl-1H-pyrazol-3-yl)methyl)-6-(phenylsulfonyl)phthalazin-1(2H)-one;
2-((5-methyl-1H-pyrazol-3-yl)methyl)-6-(phenylsulfonyl)phthalazin-1(2H)-one;
2-((3-methylisothiazol-5-yl)methyl)-6-(phenylsulfonyl)phthalazin-1(2H)-one;
2-((2-hydroxy-6-methylpyridin-3-yl)methyl)-6-(4-methoxyphenylsulfonyl)phthalazin-1(2H)-one;
2-((2-hydroxy-4-methylpyridin-3-yl)methyl)-6-(4-methoxyphenylsulfonyl)phthalazin-1(2H)-one;
2-((5-fluoro-2-hydroxypyridin-3-yl)methyl)-6-(4-methoxyphenylsulfonyl)phthalazin-1(2H)-one;
2-((2-hydroxy-5-isopropylpyridin-3-yl)methyl)-6-(4-methoxyphenylsulfonyl)phthalazin-1(2H)-one;
2-((4-chloro-2-hydroxypyridin-3-yl)methyl)-6-(4-methoxyphenylsulfonyl)phthalazin-1(2H)-one;
2-((4-fluoro-2-hydroxypyridin-3-yl)methyl)-6-(4-methoxyphenylsulfonyl)phthalazin-1(2H)-one;
6-((4-methoxyphenyl)sulfonyl)-2-((4-methyl-1H-pyrazol-3-yl)methyl)phthalazin-1(2H)-one;
2-((3-hydroxypyridin-4-yl)methyl)-6-(4-methoxyphenylsulfonyl)phthalazin-1(2H)-one;
5-methoxy-2-((6-(4-methoxyphenylsulfonyl)-1-oxophthalazin-2(1H)-yl)methyl)benzamide;
2-((4-hydroxypyridin-3-yl)methyl)-6-(4-methoxyphenylsulfonyl)phthalazin-1(2H)-one;
6-(phenylsulfonyl)-2-(pyrazolo[1,5-a]pyridin-2-ylmethyl)phthalazin-1(2H)-one;
2-((2-hydroxy-6-methoxypyridin-3-yl)methyl)-6-(4-methoxyphenylsulfonyl)phthalazin-1(2H)-one;
2-((4-fluoro-1H-pyrazol-3-yl)methyl)-6-(phenylsulfonyl)phthalazin-1(2H)-one;
2-((5-fluoro-1H-pyrazol-3-yl)methyl)-6-(phenylsulfonyl)phthalazin-1(2H)-one;
6-(4-methoxyphenylsulfonyl)-2-((3-(trifluoromethyl)isoxazol-5-yl)methyl)phthalazin-1(2H)-one;
6-(4-methoxyphenylsulfonyl)-2-((3-(trifluoromethyl)isoxazol-5-yl)methyl)phthalazin-1(2H)-one;
2-((3-hydroxypyridin-2-yl)methyl)-6-(4-methoxyphenylsulfonyl)phthalazin-1(2H)-one;
2-(3-aminobenzyl)-6-(quinolin-8-ylsulfonyl)phthalazin-1(2H)-one;
2-((6-methoxypyridin-3-yl)methyl)-6-(5-methylpyridin-2-ylsulfonyl)phthalazin-1(2H)-one;
6-(3-methoxyphenylsulfonyl)-2-((6-methoxypyridin-3-yl)methyl)phthalazin-1(2H)-one;
3-(2-((6-methoxypyridin-3-yl)methyl)-1-oxo-1,2-dihydrophthalazin-6-ylsulfonyl)thiophene-2-carboxamide;
4-(2-((6-methoxypyridin-3-yl)methyl)-1-oxo-1,2-dihydrophthalazin-6-ylsulfonyl)thiophene-2-carboxamide;
2-(3-aminobenzyl)-6-(phenylsulfinyl)phthalazine-1(2H)-one;
2-(3-aminobenzyl)-6-(phenylsulfonimidoyl)phthalazin-1(2H)-one;
2-(3-aminobenzyl)-6-(pyridin-3-ylsulfonyl)phthalazin-1(2H)-one;
2-((6-methoxypyridin-3-yl)methyl)-1-oxo-N-phenyl-1,2-dihydrophthalazine-6-sulfonamide;
6-((1H-indazol-4-yl)sulfonyl)-2-((6-methoxypyridin-3-yl)methyl)phthalazin-1(2H)-one;
6-((6-(difluoromethoxy)pyridin-3-yl)sulfonyl)-2-((6-methoxypyridin-3-yl)methyl)phthalazin-1(2H)-one;
3-((6-methoxypyridin-3-yl)methyl)-7-((3-phenyloxetan-3-yl)oxy)pyrido[3,4-d]pyridazin-4(3H)-one;
6-((2,3-dihydrobenzofuran-5-yl)sulfonyl)-2-((6-methoxypyridin-3-yl)methyl)phthalazin-1(2H)-one;
6-(2,3-dihydrobenzofuran-5-sulfonimidoyl)-2-((6-methoxypyridin-3-yl)methyl)phthalazin-1(2H)-one;
(R)-6-(2,3-dihydrobenzofuran-5-sulfonimidoyl)-2-((6-methoxypyridin-3-yl)methyl)phthalazin-1(2H)-one;
(S)-6-(2,3-dihydrobenzofuran-5-sulfonimidoyl)-2-((6-methoxypyridin-3-yl)methyl)phthalazin-1(2H)-one;
6-((2,3-dihydro-[1,4]dioxino[2,3-b]pyridin-7-yl)sulfonyl)-2-((6-methoxypyridin-3-yl)methyl)phthalazin-1(2H)-one;
2-((6-methoxypyridin-3-yl)methyl)-6-(phenylsulfonimidoyl)phthalazin-1(2H)-one;
R)-2-((6-methoxypyridin-3-yl)methyl)-6-(phenylsulfonimidoyl)phthalazin-1(2H)-one;
(S)-2-((6-methoxypyridin-3-yl)methyl)-6-(phenylsulfonimidoyl)phthalazin-1(2H)-one;
6-(4-(difluoromethoxy)phenylsulfonimidoyl)-2-((6-methoxypyridin-3-yl)methyl)phthalazin-1(2H)-one;
(R)-6-(4-(difluoromethoxy)phenylsulfonimidoyl)-2-((6-methoxypyridin-3-yl)methyl)phthalazin-1(2H)-one;
(S)-6-(4-(difluoromethoxy)phenylsulfonimidoyl)-2-((6-methoxypyridin-3-yl)methyl)phthalazin-1(2H)-one;
6-(4-methoxyphenylsulfonimidoyl)-2-((6-methoxypyridin-3-yl)methyl)phthalazin-1(2H)-one;
(R)-6-(4-methoxyphenylsulfonimidoyl)-2-((6-methoxypyridin-3-yl)methyl)phthalazin-1(2H)-one;
(S)-6-(4-methoxyphenylsulfonimidoyl)-2-((6-methoxypyridin-3-yl)methyl)phthalazin-1(2H)-one;
6-((1H-pyrazol-3-yl)sulfonyl)-2-((6-methoxypyridin-3-yl)methyl)phthalazin-1(2H)-one;
2-(3-aminobenzyl)-6-(phenylsulfonyl)phthalazin-1(2H)-one;
2-(3-aminobenzyl)-6-((4-methoxyphenyl)sulfonyl)phthalazin-1(2H)-one;
2-(3-aminobenzyl)-6-((2,3-dihydrobenzofuran-5-yl)sulfonyl)phthalazin-1(2H)-one;
2-(3-aminobenzyl)-6-(pyridin-2-ylsulfonyl)phthalazin-1(2H)-one;
2-(3-aminobenzyl)-6-((1-methyl-1H-pyrazol-3-yl)sulfonyl)phthalazin-1(2H)-one;
2-(3-aminobenzyl)-6-((5-methylthiophen-2-yl)sulfonyl)phthalazin-1(2H)-one;
6-((4-fluorophenyl)sulfonyl)-2-((6-methoxypyridin-3-yl)methyl)phthalazin-1(2H)-one;
2-((6-methoxypyridin-3-yl)methyl)-6-(3-methylisothiazol-5-ylsulfonyl)phthalazin-1(2H)-one;
2-(3-aminobenzyl)-6-((6-methoxypyridin-3-yl)sulfonyl)phthalazin-1(2H)-one;
2-((6-methoxypyridin-3-yl)methyl)-6-tosylphthalazin-1(2H)-one;
2-(3-aminobenzyl)-6-((2-methylthiazol-4-yl)sulfonyl)phthalazine-1(2H)-one;
2-(3-aminobenzyl)-6-(benzo[d][1,3]dioxol-5-ylsulfonyl)phthalazin-1(2H)-one;
6-(4-(difluoromethoxy)phenylsulfonyl)-2-((6-methoxypyridin-3-yl)methyl)phthalazin-1(2H)-one;
6-((3-chlorophenyl)sulfonyl)-2-((6-methoxypyridin-3-yl)methyl)phthalazin-1(2H)-one;
6-((4-chlorophenyl)sulfonyl)-2-((6-methoxypyridin-3-yl)methyl)phthalazin-1(2H)-one;
6-((2,2-difluorobenzo[d][1,3]dioxol-5-yl)sulfonyl)-2-((6-methoxypyridin-3-yl)methyl)phthalazin-1(2H)-one;
6-((3-fluorophenyl)sulfonyl)-2-((6-methoxypyridin-3-yl)methyl)phthalazin-1(2H)-one;
2-((6-methoxypyridin-3-yl)methyl)-6-((1-methyl-1H-pyrazol-3-yl)sulfonyl)phthalazin-1(2H)-one;
2-((6-methoxypyridin-3-yl)methyl)-6-(4-methylpyridin-2-ylsulfonyl)phthalazin-1(2H)-one;
6-(6-methoxypyridin-2-ylsulfonyl)-2-((6-methoxypyridin-3-yl)methyl)phthalazin-1(2H)-one;
3-(2-((6-methoxypyridin-3-yl)methyl)-1-oxo-1,2-dihydrophthalazin-6-ylsulfonyl)benzamide;
6-(2-fluorophenylsulfonyl)-2-((6-methoxypyridin-3-yl)methyl)phthalazin-1(2H)-one;
6-(2-chlorophenylsulfonyl)-2-((6-methoxypyridin-3-yl)methyl)phthalazin-1(2H)-one;
6-(2-methoxyphenylsulfonyl)-2-((6-methoxypyridin-3-yl)methyl)phthalazin-1(2H)-one;
6-(4-methoxypyridin-2-ylsulfonyl)-2-((6-methoxypyridin-3-yl)methyl)phthalazin-1(2H)-one;
6-((2,2-dimethyl-2,3-dihydrobenzofuran-5-yl)sulfonyl)-2-((6-methoxypyridin-3-yl)methyl)phthalazin-1(2H)-one;
2-((6-methoxypyridin-3-yl)methyl)-6-((2-methyl-2,3-dihydrobenzofuran-5-yl)sulfonyl)phthalazin-1(2H)-one;
2-((6-methoxypyridin-3-yl)methyl)-6-((1-methyl-1H-pyrazol-4-yl)sulfonyl)phthalazin-1(2H)-one;
2-((6-methoxypyridin-3-yl)methyl)-6-(6-methylpyridin-2-ylsulfonyl)phthalazin-1(2H)-one;
6-(3-(difluoromethoxy)phenylsulfonyl)-2-((6-methoxypyridin-3-yl)methyl)phthalazin-1(2H)-one;
2-((6-methoxypyridin-3-yl)methyl)-6-(5-methylthiophen-2-ylsulfonyl)phthalazin-1(2H)-one;
6-(5-methoxypyridin-2-ylsulfonyl)-2-((6-methoxypyridin-3-yl)methyl)phthalazin-1(2H)-one;
6-(2-(difluoromethyl)thiazol-5-ylsulfonyl)-2-((6-methoxypyridin-3-yl)methyl)phthalazin-1(2H)-one;
2-((6-methoxypyridin-3-yl)methyl)-6-(2-methylthiazol-4-ylsulfonyl)phthalazin-1(2H)-one;
2-((6-methoxypyridin-3-yl)methyl)-6-(6-methoxypyridin-3-ylsulfonyl)phthalazin-1(2H)-one;
2-((6-methoxypyridin-3-yl)methyl)-6-(2-methylbenzo[d]thiazol-4-ylsulfonyl)phthalazin-1(2H)-one;
6-(5-methoxypyrazin-2-ylsulfonyl)-2-((6-methoxypyridin-3-yl)methyl)phthalazin-1(2H)-one;
6-(imidazo[1,2-a]pyridin-5-ylsulfonyl)-2-((6-methoxypyridin-3-yl)methyl)phthalazin-1(2H)-one;
6-(1-(difluoromethyl)-1H-pyrazol-3-ylsulfonyl)-2-((6-methoxypyridin-3-yl)methyl)phthalazin-1(2H)-one;
(S)-6-(2,3-dihydrobenzofuran-5-sulfonimidoyl)-2-((6-methoxypyridin-3-yl)methyl)phthalazin-1(2H)-one;
6-((2,3-dihydrobenzo[b][1,4]dioxin-6-yl)sulfonyl)-2-((6-methoxypyridin-3-yl)methyl)phthalazin-1(2H)-one;
6-((2,3-dihydrofuro[2,3-b]pyridin-5-yl)sulfonyl)-2-((6-methoxypyridin-3-yl)methyl)phthalazin-1(2H)-one;
6-(imidazo[1,2-a]pyridin-7-ylsulfonyl)-2-((6-methoxypyridin-3-yl)methyl)phthalazin-1(2H)-one;
6-(1,2-dimethyl-1H-imidazol-4-ylsulfonyl)-2-((6-methoxypyridin-3-yl)methyl)phthalazin-1(2H)-one;
6-(1,5-dimethyl-1H-pyrazol-4-ylsulfonyl)-2-((6-methoxypyridin-3-yl)methyl)phthalazin-1(2H)-one;
(S)-6-(4-methoxyphenylsulfonimidoyl)-2-((6-methoxypyridin-3-yl)methyl)phthalazin-1(2H)-one;
6-(1-cyclopropyl-1H-pyrazol-4-ylsulfonyl)-2-((2-hydroxypyridin-3-yl)methyl)phthalazin-1(2H)-one;
6-(1-cyclopropyl-1H-pyrazol-4-ylsulfonyl)-2-((1-methyl-1H-pyrazol-3-yl)methyl)phthalazin-1(2H)-one;
2-((1H-pyrazol-3-yl)methyl)-6-(benzofuran-5-ylsulfonyl)phthalazin-1(2H)-one;
2-((1-cyclopropyl-1H-pyrazol-3-yl)methyl)-6-((1-cyclopropyl-1H-pyrazol-4-yl)sulfonyl)phthalazin-1(2H)-one;
6-((1H-pyrazol-4-yl)sulfonyl)-2-((6-methoxypyridin-3-yl)methyl)phthalazin-1(2H)-one;
2-(3-(difluoromethoxy)benzyl)-6-((1-methyl-1H-pyrazol-3-yl)sulfonyl)phthalazin-1(2H)-one;
2-(2-fluoro-5-methoxybenzyl)-6-((1-methyl-1H-pyrazol-3-yl)sulfonyl)phthalazin-1(2H)-one;
2-((2-hydroxypyridin-3-yl)methyl)-6-((1-methyl-1H-pyrazol-4-yl)sulfonyl)phthalazin-1(2H)-one;
6-((3,4-dihydro-2H-benzo[b][1,4]oxazin-6-yl)sulfonyl)-2-((1-methyl-1H-pyrazol-3-yl)methyl)phthalazin-1(2H)-one;
6-((3,4-dihydro-2H-benzo[b][1,4]oxazin-6-yl)sulfonyl)-2-((1-(2-hydroxyethyl)-1H-pyrazol-3-yl)methyl)phthalazin-1(2H)-one;
2-((1H-pyrazol-3-yl)methyl)-6-((2,3-dihydrobenzofuran-5-yl)sulfonyl)phthalazin-1(2H)-one;
6-((1-methyl-1H-pyrazol-4-yl)sulfonyl)-2-((6-methylpyridin-2-yl)methyl)phthalazin-1(2H)-one;
2-((1-cyclopropyl-1H-pyrazol-3-yl)methyl)-6-((6-methoxypyridin-3-yl)sulfonyl)phthalazin-1(2H)-one;
6-((2,3-dihydrobenzo[b][1,4]dioxin-6-yl)sulfonyl)-2-((1-(2-hydroxyethyl)-1H-pyrazol-3-yl)methyl)phthalazin-1(2H)-one;
2-((1H-pyrazol-3-yl)methyl)-6-((2,3-dihydrobenzo[b][1,4]dioxin-6-yl)sulfonyl)phthalazine-1(2H)-one;
6-((2,3-dihydrobenzofuran-5-yl)sulfonyl)-2-((4-methyl-1H-pyrazol-3-yl)methyl)phthalazin-1(2H)-one;
6-((2,3-dihydrobenzofuran-5-yl)sulfonyl)-2-((5-methyl-1H-pyrazol-3-yl)methyl)phthalazin-1(2H)-one;
2-((6-(1-methyl-1H-pyrazol-3-ylsulfonyl)-1-oxophthalazin-2(1H)-yl)methyl)benzamide;
2-((1H-pyrazol-3-yl)methyl)-6-(pyridin-2-ylsulfonyl)phthalazin-1(2H)-one;
6-((4-(difluoromethoxy)phenyl)sulfonyl)-2-((1-methyl-1H-pyrazol-3-yl)methyl)phthalazin-1(2H)-one;
2-((1-methyl-1H-pyrazol-3-yl)methyl)-6-((2-methyl-2,3-dihydrobenzofuran-5-yl)sulfonyl)phthalazin-1(2H)-one;
2-((1-methyl-1H-pyrazol-3-yl)methyl)-6-((2-methylthiazol-4-yl)sulfonyl)phthalazin-1(2H)-one;
6-((2,3-dihydrobenzo[b][1,4]dioxin-6-yl)sulfonyl)-2-((1-methyl-1H-pyrazol-3-yl)methyl)phthalazin-1(2H)-one;
6-((4-ethoxyphenyl)sulfonyl)-2-((1-methyl-1H-pyrazol-3-yl)methyl)phthalazin-1(2H)-one;
(R)-2-((1-methyl-1H-pyrazol-3-yl)methyl)-6-((2-methyl-2,3-dihydrobenzofuran-5-yl)sulfonyl)phthalazin-1(2H)-one;
2-((1-methyl-1H-pyrazol-3-yl)methyl)-6-((4-(trifluoromethoxy)phenyl)sulfonyl)phthalazin-1(2H)-one;
(S)-2-((1-methyl-1H-pyrazol-3-yl)methyl)-6-((2-methyl-2,3-dihydrobenzofuran-5-yl)sulfonyl)phthalazin-1(2H)-one;
2-((1H-pyrazol-3-yl)methyl)-6-((3-methylbenzofuran-5-yl)sulfonyl)phthalazin-1(2H)-one;
2-((1H-pyrazol-3-yl)methyl)-6-((3,4-dihydro-2H-benzo[b][1,4]oxazin-6-yl)sulfonyl)phthalazin-1(2H)-one;
2-((1H-pyrazol-3-yl)methyl)-6-(benzofuran-6-ylsulfonyl)phthalazin-1(2H)-one;
2-((1H-pyrazol-3-yl)methyl)-6-(furo[3,2-b]pyridin-5-ylsulfonyl)phthalazin-1(2H)-one;
2-((1H-pyrazol-3-yl)methyl)-6-((2-methylbenzofuran-5-yl)sulfonyl)phthalazin-1(2H)-one;
2-((1H-pyrazol-3-yl)methyl)-6-(furo[2,3-b]pyridin-5-ylsulfonyl)phthalazin-1(2H)-one;
6-((1H-indol-5-yl)sulfonyl)-2-((1H-pyrazol-3-yl)methyl)phthalazin-1(2H)-one;
2-((1H-pyrazol-3-yl)methyl)-6-(benzo[b]thiophen-5-ylsulfonyl)phthalazin-1(2H)-one;
2-((1H-pyrazol-3-yl)methyl)-6-((1-cyclopropyl-1H-pyrazol-4-yl)sulfonyl)phthalazin-1(2H)-one;
2-((1H-pyrazol-3-yl)methyl)-6-((2,3-dihydro-[1,4]dioxino[2,3-b]pyridin-6-yl)sulfonyl)phthalazin-1(2H)-one;
2-((1H-pyrazol-3-yl)methyl)-6-((2,3-dihydro-[1,4]dioxino[2,3-c]pyridin-7-yl)sulfonyl)phthalazin-1(2H)-one;
2-((1H-pyrazol-3-yl)methyl)-6-((1-methyl-1H-indol-5-yl)sulfonyl)phthalazin-1(2H)-one;
6-((1H-pyrazol-4-yl)sulfonyl)-2-((1-cyclopropyl-1H-pyrazol-3-yl)methyl)phthalazin-1(2H)-one;
2-((1H-pyrazol-3-yl)methyl)-6-(pyridin-3-ylsulfonyl)phthalazin-1(2H)-one;
2-((1H-pyrazol-3-yl)methyl)-6-((2,3-dihydro-[1,4]dioxino[2,3-b]pyridin-7-yl)sulfonyl)phthalazin-1(2H)-one;
2-((1H-pyrazol-3-yl)methyl)-6-((6-methoxypyridin-3-yl)sulfonyl)phthalazin-1(2H)-one;
2-((1-cyclopropyl-1H-pyrazol-3-yl)methyl)-6-((1-methyl-1H-pyrazol-4-yl)sulfonyl)phthalazin-1(2H)-one;
6-(1-(2-hydroxyethyl)-1H-pyrazol-3-ylsulfonyl)-2-((2-hydroxypyridin-3-yl)methyl)phthalazin-1(2H)-one;
6-((1H-pyrazol-4-yl)sulfonyl)-2-((6-methylpyridin-2-yl)methyl)phthalazin-1(2H)-one;
6-((1H-pyrazol-3-yl)sulfonyl)-2-((6-methylpyridin-2-yl)methyl)phthalazin-1(2H)-one;
2-((1H-pyrazol-3-yl)methyl)-6-((1-methyl-1H-pyrazol-4-yl)sulfonyl)phthalazin-1(2H)-one;
2-((1H-pyrazol-3-yl)methyl)-6-((1-(2-hydroxyethyl)-1H-pyrazol-4-yl)sulfonyl)phthalazin-1(2H)-one;
7-fluoro-2-((1-methyl-1H-pyrazol-3-yl)methyl)-6-(phenylsulfonyl)phthalazin-1(2H)-one;
5-fluoro-2-((1-methyl-1H-pyrazol-3-yl)methyl)-6-(phenylsulfonyl)phthalazin-1(2H)-one;
7-((2-hydroxyethyl)amino)-2-((1-methyl-1H-pyrazol-3-yl)methyl)-6-(phenylsulfonyl)phthalazin-1(2H)-one;
8-fluoro-2-((1-methyl-1H-pyrazol-3-yl)methyl)-6-(phenylsulfonyl)phthalazin-1(2H)-one;
7-((2-hydroxyethyl)amino)-2-((2-hydroxypyridin-3-yl)methyl)-6-(phenylsulfonyl)phthalazin-1(2H)-one;
5-((2-hydroxyethyl)amino)-2-((2-hydroxypyridin-3-yl)methyl)-6-(phenylsulfonyl)phthalazin-1(2H)-one;
3-((1-methyl-1H-pyrazol-3-yl)methyl)-7-(phenylsulfonyl)pyrido[3,4-d]pyridazin-4(3H)-one;
3-((1-methyl-1H-pyrazol-3-yl)methyl)-7-(phenylsulfinyl)pyrido[3,4-d]pyridazin-4(3H)-one;
7-((1-methyl-1H-pyrazol-3-yl)methyl)-3-(phenylsulfonyl)pyrido[2,3-d]pyridazin-8(7H)-one;
6-((1H-pyrazol-4-yl)sulfonyl)-2-((5-fluoro-6-methylpyridin-2-yl)methyl)phthalazin-1(2H)-one;
6-((1H-pyrazol-4-yl)sulfonyl)-2-((3-fluoro-6-methylpyridin-2-yl)methyl)phthalazin-1(2H)-one;
6-(1H-pyrazol-4-ylsulfonyl)-2-((2,3-dihydrofuro[3,2-b]pyridin-5-yl)methyl)phthalazin-1(2H)-one;
2-((1H-pyrazol-3-yl)methyl)-6-(pyridin-4-ylsulfonyl)phthalazin-1(2H)-one;
2-((2,4-dihydroxypyridin-3-yl)methyl)-6-(4-methoxyphenylsulfonyl)phthalazin-1(2H)-one;
2-((1-cyclopropyl-1H-pyrazol-3-yl)methyl)-6-(furo[3,2-b]pyridin-5-ylsulfonyl)phthalazin-1(2H)-one;
2-((1H-pyrazol-3-yl)methyl)-6-((5-methoxypyridin-2-yl)sulfonyl)phthalazin-1(2H)-one;
2-((1H-pyrazol-3-yl)methyl)-6-((2,3-dihydrofuro[2,3-c]pyridin-5-yl)sulfonyl)phthalazin-1(2H)-one;
6-((1-cyclopropyl-1H-pyrazol-4-yl)sulfonyl)-2-((6-methylpyridin-2-yl)methyl)phthalazin-1(2H)-one;
2-((1H-pyrazol-3-yl)methyl)-6-(pyrazin-2-ylsulfonyl)phthalazin-1(2H)-one;
2-(imidazo[1,2-a]pyridin-3-ylmethyl)-6-(phenylsulfonyl)phthalazin-1(2H)-one;
2-((1-methyl-1H-pyrazol-3-yl)methyl)-6-(phenylsulfonimidoyl)phthalazin-1(2H)-one;
2-((1H-pyrazol-3-yl)methyl)-7-((2-hydroxyethyl)amino)-6-(phenylsulfonyl)phthalazin-1(2H)-one;
6-((1-(2-hydroxyethyl)-1H-pyrazol-4-yl)sulfonyl)-2-((1-methyl-1H-pyrazol-3-yl)methyl)phthalazin-1(2H)-one;
2-((1-cyclopropyl-1H-pyrazol-3-yl)methyl)-7-((2-hydroxyethyl)amino)-6-(phenylsulfonyl)phthalazin-1(2H)-one;
3-((1H-pyrazol-3-yl)methyl)-7-((1-methyl-1H-pyrazol-4-yl)sulfonyl)pyrido[3,4-d]pyridazin-4(3H)-one;
7-((1H-pyrazol-3-yl)methyl)-3-((1-methyl-1H-pyrazol-4-yl)sulfonyl)pyrido[2,3-d]pyridazin-8(7H)-one;
2-((1H-pyrazol-3-yl)methyl)-7-((2-hydroxyethyl)amino)-6-((1-methyl-1H-pyrazol-4-yl)sulfonyl)phthalazin-1(2H)-one;
2-((1H-pyrazol-3-yl)methyl)-6-((1-(2-hydroxyethyl)-1H-pyrazol-4-yl)sulfonyl)phthalazin-1(2H)-one;
2-((6-methylpyridin-2-yl)methyl)-6-((1-(oxetan-3-yl)-1H-pyrazol-4-yl)sulfonyl)phthalazin-1(2H)-one;
6-((1-(2-hydroxyethyl)-1H-pyrazol-4-yl)sulfonyl)-2-((6-methoxypyridin-3-yl)methyl)phthalazin-1(2H)-one;
2-((1-cyclopropyl-1H-pyrazol-3-yl)methyl)-6-((1-(2-hydroxyethyl)-1H-pyrazol-4-yl)sulfonyl)phthalazin-1(2H)-one;
2-((1-(oxetan-3-yl)-1H-pyrazol-3-yl)methyl)-6-(phenylsulfonyl)phthalazin-1(2H)-one;
2-((6-(1-hydroxyethyl)pyridin-2-yl)methyl)-6-((1-methyl-1H-pyrazol-4-yl)sulfonyl)phthalazin-1(2H)-one;
2-((6-cyclopropylpyridin-2-yl)methyl)-6-((1-methyl-1H-pyrazol-4-yl)sulfonyl)phthalazin-1(2H)-one;
6-((1-(azetidin-3-yl)-1H-pyrazol-4-yl)sulfonyl)-2-((6-methylpyridin-2-yl)methyl)phthalazin-1(2H)-one;
6-((1-(2-aminoethyl)-1H-pyrazol-4-yl)sulfonyl)-2-((6-methylpyridin-2-yl)methyl)phthalazin-1(2H)-one;
(S)-2-((6-(1-hydroxyethyl)pyridin-2-yl)methyl)-6-((1-methyl-1H-pyrazol-4-yl)sulfonyl)phthalazin-1(2H)-one;
(R)-2-((6-(1-hydroxyethyl)pyridin-2-yl)methyl)-6-((1-methyl-1H-pyrazol-4-yl)sulfonyl)phthalazin-1(2H)-one;
6-((1-(2-hydroxyethyl)-1H-pyrazol-3-yl)sulfonyl)-2-((6-methoxypyridin-3-yl)methyl)phthalazin-1(2H)-one;
2-((1H-pyrazol-3-yl)methyl)-6-((1-(2-methoxyethyl)-1H-pyrazol-4-yl)sulfonyl)phthalazin-1(2H)-one;
6-((2-(1-hydroxyethyl)thiazol-4-yl)sulfonyl)-2-((6-methylpyridin-2-yl)methyl)phthalazin-1(2H)-one;
2-((6-(3-hydroxyoxetan-3-yl)pyridin-2-yl)methyl)-6-((1-methyl-1H-pyrazol-4-yl)sulfonyl)phthalazin-1(2H)-one;
6-((1H-pyrazol-4-yl)sulfonyl)-2-((2-methoxypyridin-3-yl)methyl)phthalazin-1(2H)-one;
2-((1-(2-hydroxyethyl)-1H-pyrazol-3-yl)methyl)-6-((6-methoxypyridin-3-yl)sulfonyl)phthalazin-1(2H)-one;
2-((1H-pyrazol-3-yl)methyl)-6-((6-methoxypyridin-2-yl)sulfonyl)phthalazin-1(2H)-one;
6-((1H-pyrazol-4-yl)sulfonyl)-2-((6-cyclopropylpyridin-2-yl)methyl)phthalazin-1(2H)-one;
2-((1H-pyrazol-3-yl)methyl)-6-((6-methylpyridin-2-yl)sulfonyl)phthalazin-1(2H)-one;
2-((1H-pyrazol-3-yl)methyl)-6-((5-methylpyridin-2-yl)sulfonyl)phthalazin-1(2H)-one;
2-((1H-pyrazol-3-yl)methyl)-6-((6-methylpyridin-3-yl)sulfonyl)phthalazin-1(2H)-one;
6-(1H-pyrazol-4-ylsulfonyl)-2-((5-methoxy-6-methylpyridin-2-yl)methyl)phthalazin-1(2H)-one;
2-((1-(2-hydroxyethyl)-1H-pyrazol-3-yl)methyl)-6-((5-methoxypyridin-2-yl)sulfonyl)phthalazin-1(2H)-one;
6-(1H-pyrazol-4-ylsulfonyl)-2-((2,3-dihydrofuro[2,3-b]pyridin-5-yl)methyl)phthalazin-1(2H)-one;
6-((1H-pyrazol-4-yl)sulfonyl)-2-((6-methoxypyridin-2-yl)methyl)phthalazin-1(2H)-one;
6-((1H-pyrazol-4-yl)sulfonyl)-2-((6-methylpyridin-3-yl)methyl)phthalazin-1(2H)-one;
(R)-6-((2-(1-hydroxyethyl)thiazol-4-yl)sulfonyl)-2-((6-methylpyridin-2-yl)methyl)phthalazin-1(2H)-one;
(S)-6-((2-(1-hydroxyethyl)thiazol-4-yl)sulfonyl)-2-((6-methylpyridin-2-yl)methyl)phthalazin-1(2H)-one;
6-((1H-pyrazol-4-yl)sulfonyl)-2-(furo[3,2-b]pyridin-5-ylmethyl)phthalazin-1(2H)-one;
6-((1H-pyrazol-3-yl)sulfonyl)-2-((2,3-dihydrofuro[3,2-b]pyridin-5-yl)methyl)phthalazin-1(2H)-one;
7-((1H-pyrazol-4-yl)sulfonyl)-3-((5-methyl-1H-pyrazol-3-yl)methyl)pyrido[3,4-d]pyridazin-4(3H)-one;
6-((1H-pyrazol-5-yl)sulfonyl)-2-((3-fluoro-6-methylpyridin-2-yl)methyl)phthalazin-1(2H)-one;
6-((1H-pyrazol-5-yl)sulfonyl)-2-((6-cyclopropylpyridin-2-yl)methyl)phthalazin-1(2H)-one;
6-((1H-pyrazol-5-yl)sulfonyl)-2-((5-fluoro-6-methylpyridin-2-yl)methyl)phthalazin-1(2H)-one;
6-((1H-pyrazol-4-yl)sulfonyl)-2-((5-methoxypyridin-2-yl)methyl)phthalazin-1(2H)-one;
2-((1H-pyrazol-3-yl)methyl)-6-((1-(difluoromethyl)-1H-pyrazol-4-yl)sulfonyl)phthalazin-1(2H)-one;
2-((1H-pyrazol-3-yl)methyl)-6-((1-methyl-1H-pyrazol-3-yl)sulfonyl)phthalazin-1(2H)-one;
2-((6-cyclopropylpyridin-2-yl)methyl)-6-((1-(2-hydroxyethyl)-1H-pyrazol-3-yl)sulfonyl)phthalazin-1(2H)-one;
6-((1H-pyrazol-4-yl)sulfonyl)-2-((4-chloro-1-methyl-1H-pyrazol-3-yl)methyl)phthalazin-1(2H)-one;
2-((2,3-dihydrofuro[3,2-b]pyridin-5-yl)methyl)-6-((1-(2-hydroxyethyl)-1H-pyrazol-4-yl)sulfonyl)phthalazin-1(2H)-one;
6-((1H-pyrazol-4-yl)sulfonyl)-2-((1,5-dimethyl-1H-pyrazol-3-yl)methyl)phthalazin-1(2H)-one;
6-((1-(2-hydroxyethyl)-1H-pyrazol-4-yl)sulfonyl)-2-((5-methoxypyridin-2-yl)methyl)phthalazin-1(2H)-one;
2-((5-methyl-1H-pyrazol-3-yl)methyl)-6-((1-methyl-1H-pyrazol-4-yl)sulfonyl)phthalazin-1(2H)-one;
2-((4-methyl-1H-pyrazol-3-yl)methyl)-6-((1-methyl-1H-pyrazol-4-yl)sulfonyl)phthalazin-1(2H)-one;
6-((1H-pyrazol-4-yl)sulfonyl)-2-((4-chloro-5-methyl-1H-pyrazol-3-yl)methyl)phthalazin-1(2H)-one;
6-((1-(difluoromethyl)-1H-pyrazol-4-yl)sulfonyl)-2-((5-methyl-1H-pyrazol-3-yl)methyl)phthalazin-1(2H)-one;
6-((4-fluoro-1H-pyrazol-3-yl)sulfonyl)-2-((6-methylpyridin-2-yl)methyl)phthalazin-1(2H)-one;
2-((5-chloro-1H-pyrazol-3-yl)methyl)-6-((1-methyl-1H-pyrazol-4-yl)sulfonyl)phthalazin-1(2H)-one;
6-((3-chloro-1H-pyrazol-4-yl)sulfonyl)-2-((6-methylpyridin-2-yl)methyl)phthalazin-1(2H)-one;
6-((4-chloro-1H-pyrazol-3-yl)sulfonyl)-2-((6-methylpyridin-2-yl)methyl)phthalazin-1(2H)-one;
2-((1H-pyrazol-3-yl)methyl)-6-((2-methylthiazol-4-yl)sulfonyl)phthalazin-1(2H)-one;
6-((1H-pyrazol-4-yl)sulfonyl)-2-((3-fluoro-5,6-dimethylpyridin-2-yl)methyl)phthalazin-1(2H)-one;
2-((5-fluoro-1H-pyrazol-3-yl)methyl)-6-((1-methyl-1H-pyrazol-4-yl)sulfonyl)phthalazin-1(2H)-one;
6-((1H-pyrazol-4-yl)sulfonyl)-2-((2,3-dihydropyrazolo[5,1-b]oxazol-6-yl)methyl)phthalazin-1(2H)-one;
2-((2,3-dihydropyrazolo[5,1-b]oxazol-6-yl)methyl)-6-((1-methyl-1H-pyrazol-4-yl)sulfonyl)phthalazin-1(2H)-one;
6-((1H-pyrazol-3-yl)sulfonyl)-2-((5-methoxypyridin-2-yl)methyl)phthalazin-1(2H)-one;
6-((1H-pyrazol-4-yl)sulfonyl)-2-((3-fluoro-6-methoxypyridin-2-yl)methyl)phthalazin-1(2H)-one;
2-((2,3-dihydrofuro[3,2-b]pyridin-5-yl)methyl)-6-((2-(1-hydroxyethyl)thiazol-4-yl)sulfonyl)phthalazin-1(2H)-one;
6-((1H-pyrazol-3-yl)sulfonyl)-2-((1-(difluoromethyl)-1H-pyrazol-3-yl)methyl)phthalazin-1(2H)-one;
6-((1H-pyrazol-3-yl)sulfonyl)-2-((3-fluoro-6-methoxypyridin-2-yl)methyl)phthalazin-1(2H)-one;
2-(furo[3,2-b]pyridin-5-ylmethyl)-6-((1-methyl-1H-pyrazol-4-yl)sulfonyl)phthalazin-1(2H)-one;
3-((5-methyl-1H-pyrazol-3-yl)methyl)-7-((1-methyl-1H-pyrazol-4-yl)sulfonyl)pyrido[3,4-d]pyridazin-4(3H)-one;
6-((1H-pyrazol-3-yl)sulfonyl)-2-((4-chloro-5-methyl-1H-pyrazol-3-yl)methyl)phthalazin-1(2H)-one;
2-((2,3-dihydrofuro[3,2-b]pyridin-5-yl)methyl)-6-((1-(2-methoxyethyl)-1H-pyrazol-4-yl)sulfonyl)phthalazin-1(2H)-one;
2-((2,3-dihydrofuro[3,2-b]pyridin-5-yl)methyl)-6-((1-(oxetan-3-yl)-1H-pyrazol-4-yl)sulfonyl)phthalazin-1(2H)-one;
6-((1H-pyrazol-3-yl)sulfonyl)-2-(furo[3,2-b]pyridin-5-ylmethyl)phthalazin-1(2H)-one;
2-(furo[3,2-b]pyridin-5-ylmethyl)-6-((1-methyl-1H-pyrazol-3-yl)sulfonyl)phthalazin-1(2H)-one;
2-((4-chloro-5-methyl-1H-pyrazol-3-yl)methyl)-6-((1-methyl-1H-pyrazol-3-yl)sulfonyl)phthalazin-1(2H)-one;
6-((1H-pyrazol-4-yl)sulfonyl)-2-((4-chloro-1,5-dimethyl-1H-pyrazol-3-yl)methyl)phthalazin-1(2H)-one;
6-((1H-pyrazol-5-yl)sulfonyl)-2-((1-cyclopropyl-1H-pyrazol-3-yl)methyl)phthalazin-1(2H)-one;
6-((1-(difluoromethyl)-1H-pyrazol-4-yl)sulfonyl)-2-((2,3-dihydrofuro[3,2-b]pyridin-5-yl)methyl)phthalazin-1(2H)-one;
2-((2,3-dihydrofuro[3,2-b]pyridin-5-yl)methyl)-6-(1H-pyrazole-4-sulfonimidoyl)phthalazin-1(2H)-one;
6-((1H-pyrazol-4-yl)sulfonyl)-2-((3,5-difluoro-6-methylpyridin-2-yl)methyl)phthalazin-1(2H)-one;
7-((1-(difluoromethyl)-1H-pyrazol-4-yl)sulfonyl)-3-((5-methyl-1H-pyrazol-3-yl)methyl)pyrido[3,4-d]pyridazin-4(3H)-one;
6-((1H-pyrazol-4-yl)sulfonyl)-2-((2,3-dihydro-[1,4]dioxino[2,3-b]pyridin-6-yl)methyl)phthalazin-1(2H)-one;
6-((1H-pyrazol-4-yl)sulfonyl)-2-((2-methylfuro[3,2-b]pyridin-5-yl)methyl)phthalazin-1(2H)-one;
2-((1H-pyrazol-3-yl)methyl)-6-((2-cyclopropylthiazol-4-yl)sulfonyl)phthalazin-1(2H)-one;
7-((1H-pyrazol-4-yl)sulfonyl)-3-((2,3-dihydrofuro[3,2-b]pyridin-5-yl)methyl)pyrido[3,4-d]pyridazin-4(3H)-one;
2-((4,5-dimethyl-1H-pyrazol-3-yl)methyl)-6-((1-methyl-1H-pyrazol-4-yl)sulfonyl)phthalazin-1(2H)-one;
2-((4-chloro-5-methyl-1H-pyrazol-3-yl)methyl)-6-((1-(difluoromethyl)-1H-pyrazol-4-yl)sulfonyl)phthalazin-1(2H)-one;
2-((2,3-dihydrofuro[3,2-b]pyridin-5-yl)methyl)-6-((2-(1-methoxyethyl)thiazol-4-yl)sulfonyl)phthalazin-1(2H)-one;
2-((1H-pyrazol-3-yl)methyl)-6-((2-(1-methoxyethyl)thiazol-4-yl)sulfonyl)phthalazin-1(2H)-one;
2-(furo[3,2-b]pyridin-5-ylmethyl)-6-((2-(1-methoxyethyl)thiazol-4-yl)sulfonyl)phthalazin-1(2H)-one;
2-((3-fluoro-5,6-dimethylpyridin-2-yl)methyl)-6-((2-(1-methoxyethyl)thiazol-4-yl)sulfonyl)phthalazin-1(2H)-one;
6-((1H-pyrazol-4-yl)sulfonyl)-2-((3,4-dihydro-2H-pyrido[3,2-b][1,4]oxazin-6-yl)methyl)phthalazin-1(2H)-one;
6-((1-methyl-1H-pyrazol-4-yl)sulfonyl)-2-((7-methyl-2,3-dihydropyrazolo[5,1-b]oxazol-6-yl)methyl)phthalazin-1(2H)-one;
2-((2,3-dihydropyrazolo[5,1-b]oxazol-6-yl)methyl)-6-((2-(1-methoxyethyl)thiazol-4-yl)sulfonyl)phthalazin-1(2H)-one;
2-((2,3-dihydropyrazolo[5,1-b]oxazol-6-yl)methyl)-6-((2-methylthiazol-4-yl)sulfonyl)phthalazin-1(2H)-one;
2-((5-methyl-1H-pyrazol-3-yl)methyl)-6-((2-methylthiazol-4-yl)sulfonyl)phthalazin-1(2H)-one;
2-((2,3-dihydrofuro[3,2-b]pyridin-5-yl)methyl)-6-((5-methyl-1H-pyrazol-3-yl)sulfonyl)phthalazin-1(2H)-one;
2-((2,3-dihydrofuro[3,2-b]pyridin-5-yl)methyl)-6-((1-(2-methoxyethyl)-1H-pyrazol-3-yl)sulfonyl)phthalazin-1(2H)-one;
2-((1H-pyrazol-3-yl)methyl)-6-((1-(2,2,2-trifluoroethyl)-1H-pyrazol-4-yl)sulfonyl)phthalazin-1(2H)-one;
2-[(7-methyl-2,3-dihydropyrazolo[5,1-b]oxazol-6-yl)methyl]-6-(1H-pyrazol-4-ylsulfonyl)phthalazin-1-one;
(R)-2-((2,3-dihydrofuro[3,2-b]pyridin-5-yl)methyl)-6-(1H-pyrazole-4-sulfonimidoyl)phthalazin-1(2H)-one; and
(S)-2-((2,3-dihydrofuro[3,2-b]pyridin-5-yl)methyl)-6-(1H-pyrazole-4-sulfonimidoyl)phthalazin-1(2H)-one;
or a pharmaceutically acceptable salt and/or solvate thereof.

152: A pharmaceutical composition comprising a compound, pharmaceutically acceptable salt and/or solvate thereof according to claim 1, and one or more pharmaceutically acceptable diluents or carriers.

153: A method of treating or preventing a disease, disorder or condition associated with the function of PK, in particular PKM2 and/or PKLR, which comprises administering a compound, pharmaceutically acceptable salt and/or solvate thereof according to claim 1.

154: The method according to claim 153, for treating or preventing an inflammatory disease, a disease associated with an undesirable immune response, cancer, obesity, a diabetic disease or a blood disorder.

155: The method according to claim 154, wherein the inflammatory disease or disease associated with an undesirable immune response is, or is associated with, a disease selected from the group consisting of: psoriasis, asthma, chronic obstructive pulmonary disease (COPD), heart failure, myocardial infarction, angina pectoris, other atherosclerosis and/or atherothrombosis-related disorders, a mitochondrial and neurodegenerative disease, autoimmune paraneoplastic retinopathy, transplantation rejection, multiple sclerosis, transverse myelitis, ischaemia-reperfusion injury, AGE-induced genome damage, an inflammatory bowel disease, primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes, eczema, sarcoidosis, erythema nodosum, acute disseminated encephalomyelitis (ADEM), neuromyelitis optica spectrum disorders, MOG (myelin oligodendrocyte glycoprotein) antibody-associated disorders, optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides, antiphospholipid antibody syndrome, autoimmune hemolytic anaemia, autoimmune hepatitis, autoimmune inner ear disease, pemphigoid, linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes, progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis, narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD), hepatitis C (HCV) infection or complication, herpes simplex viral infection or complication, human immunodeficiency virus (HIV) infection or complication, lichen planus, monomelic amyotrophy, fibrosis, cystic fibrosis, pulmonary arterial hypertension (PAH), lung sarcoidosis, idiopathic pulmonary fibrosis, kidney fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration, post-operative cataract inflammation, uveitis, iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis, adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS), paraneoplastic syndromes, perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid, eosinophilic fasciitis (Shulman syndrome) progesterone hypersensitivity, Familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies, Schnitzler syndrome; familial cylindromatosis, congenital B cell lymphocytosis, OTULIN-related autoinflammatory syndrome, type 2 diabetes mellitus, insulin resistance and the metabolic syndrome, atherosclerotic disorders, and renal inflammatory disorders.

156: The method according to claim 155, wherein the inflammatory disease or disease associated with an undesirable immune response is selected from the group consisting of rheumatoid arthritis, psoriatic arthritis, ankylosing spondylitis, systemic lupus erythematosus, multiple sclerosis, psoriasis, inflammatory bowel disease, atopic dermatitis, fibrosis, uveitis, cryopyrin-associated periodic syndromes, Muckle-Wells syndrome, juvenile idiopathic arthritis, chronic obstructive pulmonary disease and asthma.

157: The method according to claim 154, wherein cancer is selected from the group consisting of acute lymphoblastic leukaemia, adult; acute lymphoblastic leukaemia, childhood; acute myeloid leukaemia, adult; adrenocortical carcinoma; adrenocortical carcinoma, childhood; aids-related lymphoma; aids-related malignancies; anal cancer; astrocytoma, childhood cerebellar; astrocytoma, childhood cerebral; Barrett's esophagus (pre-malignant syndrome); bile duct cancer, extrahepatic; bladder cancer; bladder cancer, childhood; bone cancer, osteosarcoma/malignant fibrous histiocytoma; brain stem glioma, childhood; brain tumour, adult; brain tumour, brain stem glioma, childhood; brain tumour, cerebellar astrocytoma, childhood; brain tumour, cerebral astrocytoma/malignant glioma, childhood; brain tumour, ependymoma, childhood; brain tumour, medulloblastoma, childhood; brain tumour, supratentorial primitive neuroectodermal tumours, childhood; brain tumour, visual pathway and hypothalamic glioma, childhood; brain tumour, childhood (other); breast cancer; breast cancer and pregnancy; breast cancer, childhood; breast cancer, male; bronchial adenomas/carcinoids, childhood; carcinoid tumour, childhood; carcinoid tumour, gastrointestinal; carcinoma, adrenocortical; carcinoma, islet cell; carcinoma of unknown primary; central nervous system lymphoma, primary; cerebellar astrocytoma, childhood; cerebral astrocytoma/malignant glioma, childhood; cervical cancer; childhood cancers; chronic lymphocytic leukaemia; chronic myelogenous leukaemia; chronic myeloproliferative disorders; clear cell sarcoma of tendon sheaths; colon cancer; colorectal cancer; colorectal cancer, childhood; cutaneous t-cell lymphoma; endometrial cancer; ependymoma, childhood; epithelial cancer, ovarian; oesophageal cancer; oesophageal cancer, childhood; Ewing's family of tumours; extracranial germ cell tumour, childhood; extragonadal germ cell tumour; extrahepatic bile duct cancer; eye cancer, intraocular melanoma; eye cancer, retinoblastoma; gallbladder cancer; gastric (stomach) cancer; gastric (stomach) cancer, childhood; gastrointestinal carcinoid tumour; germ cell tumour, extracranial, childhood; germ cell tumour, extragonadal; germ cell tumour, ovarian; gestational trophoblastic tumour; glioma, childhood brain stem; glioma, childhood visual pathway and hypothalamic; hairy cell leukaemia; head and neck cancer; hepatocellular (liver) cancer; hepatocellular (liver) cancer, adult (primary); hepatocellular (liver) cancer, childhood (primary); cancer of the esophagus; Hodgkin's lymphoma; Hodgkin's lymphoma, adult; Hodgkin's lymphoma, childhood; Hodgkin's lymphoma during pregnancy; hypopharyngeal cancer; hypothalamic and visual pathway glioma, childhood; intraocular melanoma; islet cell carcinoma (endocrine pancreas); cancer of the endocrine system; Kaposi's sarcoma; kidney cancer; laryngeal cancer; laryngeal cancer, childhood; leukaemia, acute lymphoblastic, adult; leukaemia, acute lymphoblastic, childhood; leukaemia, acute myeloid, adult; leukaemia, acute myeloid, childhood; leukaemia, chronic lymphocytic; leukaemia, chronic myelogenous; leukaemia, hairy cell; lymphocytic lymphoma; lip and oral cavity cancer; liver cancer, adult (primary); liver cancer, childhood (primary); lung cancer; lung cancer, non-small cell; lung cancer, small cell; lymphoblastic leukaemia, adult acute; lymphoblastic leukaemia, childhood acute; lymphocytic leukaemia, chronic; lymphoma, aids-related; lymphoma, central nervous system (primary); lymphoma, cutaneous t-cell; lymphoma, Hodgkin's, adult; lymphoma, Hodgkin's, childhood; lymphoma, Hodgkin's during pregnancy; lymphoma, non-Hodgkin's, adult; lymphoma, non-Hodgkin's, childhood; lymphoma, non-Hodgkin's during pregnancy; lymphoma, primary central nervous system; macroglobulinemia, Waldenstrom's; male breast cancer; malignant mesothelioma, adult; malignant mesothelioma, childhood; malignant thymoma; medulloblastoma, childhood; melanoma; melanoma, intraocular; Merkel cell carcinoma; mesothelioma, malignant; metastatic squamous neck cancer with occult primary; multiple endocrine neoplasia syndrome, childhood; multiple myeloma/plasma cell neoplasm; mycosis fungoides; myelodysplastic syndromes; myelogenous leukaemia, chronic; myeloid leukaemia, childhood acute; myeloma, multiple; myeloproliferative disorders, chronic; nasal cavity and paranasal sinus cancer; nasopharyngeal cancer; nasopharyngeal cancer, childhood; neoplastic cutaneous disease; neuroblastoma; non-Hodgkin's lymphoma, adult; non-Hodgkin's lymphoma, childhood; non-Hodgkin's lymphoma during pregnancy; non-small cell lung cancer; neoplasms of the central nervous system; oat-cell cancer; oral cancer, childhood; oral cavity and lip cancer; oropharyngeal cancer; osteosarcoma/malignant fibrous histiocytoma of bone; ovarian cancer; ovarian cancer, childhood; ovarian epithelial cancer; ovarian germ cell tumour; ovarian low malignant potential tumour; pediatric malignancy; pancreatic cancer; pancreatic cancer, childhood; pancreatic cancer, islet cell; paranasal sinus and nasal cavity cancer; parathyroid cancer; penile cancer; pheochromocytoma; pineal and supratentorial primitive neuroectodermal tumours, childhood; pituitary tumour; plasma cell neoplasm/multiple myeloma; pleuropulmonary blastoma; pregnancy and breast cancer; pregnancy and Hodgkin's lymphoma; pregnancy and non-Hodgkin's lymphoma; primary central nervous system lymphoma; primary liver cancer, adult; primary liver cancer, childhood; prostate cancer (particularly hormone-refractory); chronic or acute leukemia; solid tumors of childhood; hypereosinophilia; rectal cancer; renal cell (kidney) cancer; renal cell cancer, childhood; renal pelvis and ureter, transitional cell cancer; retinoblastoma; rhabdomyosarcoma, childhood; salivary gland cancer; salivary gland cancer, childhood; sarcoma, Ewing's family of tumours; sarcoma, Kaposi's; sarcoma (osteosarcoma)/malignant fibrous histiocytoma of bone; sarcoma, rhabdomyosarcoma, childhood; sarcomas of soft tissues; sarcoma, soft tissue, adult; sarcoma, soft tissue, childhood; Sezary syndrome; skin cancer; skin cancer, childhood; skin cancer (melanoma); skin carcinoma, Merkel cell; small cell lung cancer; dermatofibrosarcoma protuberans; small intestine cancer; soft tissue sarcoma, adult; soft tissue sarcoma, childhood; cancer of the head and neck; squamous neck cancer with occult primary, metastatic; stomach (gastric) cancer; stomach (gastric) cancer, childhood; supratentorial primitive neuroectodermal tumours, childhood; t-cell lymphoma, cutaneous; testicular cancer; thymoma, childhood; thymoma, malignant; thyroid cancer; thyroid cancer, childhood; transitional cell cancer of the renal pelvis and ureter; trophoblastic tumour, gestational; unknown primary site, cancer of, childhood; unusual cancers of childhood; ureter and renal pelvis, transitional cell cancer; urethral cancer; cancer of the ureter; cancer of the penis; gynecologic tumors; uterine cancer; uterine sarcoma; carcinoma of the fallopian tubes; carcinoma of the endometrium; vaginal cancer; carcinoma of the vagina; carcinoma of the vulva; visual pathway and hypothalamic glioma, childhood; vulvar cancer; Waldenstrom's macro globulinemia; and Wilms' tumour.

158: The method according to claim 155, for use in combination with a further therapeutic agent selected from the group consisting of a corticosteroid (glucocorticoid), retinoid, anthralin, vitamin D analogue, calcineurin inhibitors, phototherapy or photochemotherapy or other form of ultraviolet light irradiation therapy, ciclosporine, a thiopurine, methotrexate, an anti-TNFα agent, phosphodiesterase-4 (PDE4) inhibitors, anti-IL-17 agent, anti-IL12/IL-23 agent, anti-IL-23 agent, JAK (Janus Kinase) inhibitor, plasma exchange, intravenous immune globulin (IVIG), cyclophosphamide, anti-CD20 B cell depleting agent, anthracycline analogue, cladribine, sphingosine 1-phosphate receptor modulator or sphingosine analogue, interferon beta preparation, glatiramer, anti-CD3 therapy, anti-CD52 targeting agent, leflunomide, teriflunomide, gold compound, laquinimod, potassium channel blocker, mycophenolic acid, mycophenolate mofetil, purine analogue, mTOR (mechanistic target of rapamycin) pathway inhibitor, anti-thymocyte globulin (ATG), IL-2 receptor (CD25) inhibitor, anti-IL-6 receptor or anti-IL-6 agent, Bruton's tyrosine kinase (BTK) inhibitor, tyrosine kinase inhibitor, ursodeoxycholic acid, hydroxychloroquine, chloroquine, B cell activating factor (BAFF, also known as BlyS, B lymphocyte stimulator) inhibitor, other B cell targeted therapy including a fusion protein targeting both APRIL (A Proliferation-Inducing Ligand) and BlyS, PI3K inhibitor including pan-inhibitor or one targeting the p110δ and/or p110γ containing isoforms, an interferon α receptor inhibitor, T cell co-stimulation blocker, thalidomide and its derivatives, dapsone, clofazimine, a leukotriene antagonist, theophylline, anti-IgE therapy, an anti-IL-5 agent, a long-acting muscarinic agent, a PDE4 inhibitor, riluzole, a free radical scavenger, a proteasome inhibitor, a complement cascade inhibitor including one directed against C5, immunoadsor, antithymocyte globulin, 5-aminosalicylates and their derivatives, an anti-integrin agent including one targeting α4β1 and/or α4β7 integrins, an anti-CD11-α agent, a non-steroidal anti-inflammatory drug (NSAID) including a salicylate, a propionic acid, an acetic acid, an oxicam, a fenamate, a selective or relatively selective COX-2 inhibitor, colchicine, an IL-4 receptor inhibitor, topical/contact immunotherapy, anti-IL-1 receptor therapy, IL-1β inhibitor, IL-1 neutralising therapy, chlorambucil, a specific antibiotic with immunomodulatory properties and/or ability to modulate NRF2, anti-androgenic therapy, pentoxifylline, ursodeoxycholic acid, obeticholic acid, fibrate, a cystic fibrosis transmembrane conductance regulator (CFTR) modulator, a VEGF (vascular endothelial growth factor) inhibitor, pirfenidone and mizoribine.

159: A process for preparing a compound of formula (Ia) as described in claim 115, or a salt, such as a pharmaceutically acceptable salt and/or solvate thereof, which comprises reacting a compound of formula (IIa-S): 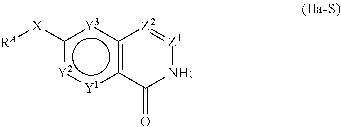
or a salt thereof;
with a compound of formula (IIIa-S): 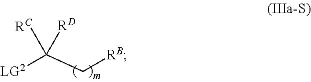
wherein R^{A}, R^{B}, R^{C}, R^{D} Y¹, Y², Y³, Z¹, Z², X and m are defined according to claim 1, and LG² is a leaving group such as halo, OMs or OTs.

160: A compound selected from the group consisting of:
a compound of formula (IIa-S): 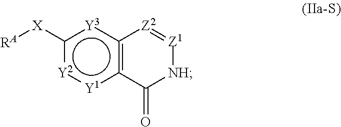
or a salt and/or solvate thereof;
wherein R^{A} Y¹, Y², Y³, Z¹, Z² and X are defined according to claim 1;
a compound of formula (IVa): 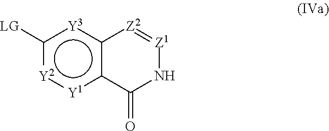
or a salt and/or solvate thereof;
wherein Y¹, Y², Y³, Z¹ and Z² are defined in claim 1, and LG is a leaving group such as halo, OMs or OTs;
a compound of formula (Va): 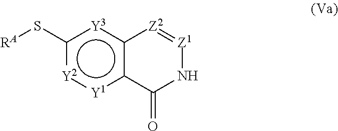
or a salt and/or solvate thereof;
wherein R^{A} Y¹, Y², Y³, Z¹, and Z² are defined in claim 1;
a compound of formula (VIa): 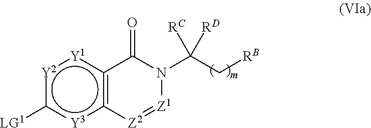
or a salt and/or solvate thereof;
wherein R^{B}, R^{C}, R^{D}, m, Y¹, Y², Y³, Z¹ and Z² are defined in claim 1 and LG¹ is a leaving group such as halo, OMs or OTs; and
a compound of formula (VIIa): 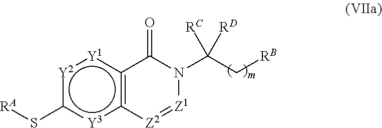
or a salt and/or solvate thereof;
wherein R^{A}, R^{B}, R^{C}, R^{D}, m, Y¹, Y², Y³, Z¹ and Z² are defined in claim 1.

161: The compound or a pharmaceutically acceptable salt and/or solvate thereof according to claim 115 which is a pharmaceutically acceptable salt of the compound of formula (Ia).

162: The compound or a pharmaceutically acceptable salt and/or solvate thereof according to claim 115 which is the compound of formula (Ia).

163. The compound, pharmaceutically acceptable salt and/or solvate thereof according to claim 115, wherein R^{C} is H and R^{D} is H.
